(19) United States
(12) Patent Application Publication
(10) Pub. No.: US 2006/0269957 A1
(43) Pub. Date: Nov. 30, 2006

(54) Secreted proteins and polynucleotides encoding them

(75) Inventors: Kenneth Jacobs, Newton, MA (US); John M. McCoy, Reading, MA (US); Edward R. LaVallie, Harvard, MA (US); Lisa A. Collins-Racie, Acton, MA (US); Cheryl Evans, Germantown, MD (US); David Merberg, Acton, MA (US); Maurice Treacy, Dublin (IE); Michael R. Bowman, Canton, MA (US); Vikki Spaulding, Danville, NH (US); McKeough Carlin-Duckett, Stoneham, MA (US); Kerry Kelleher, Marlborough, MA (US)

Correspondence Address: FITZPATRICK CELLA HARPER & SCINTO, 30 ROCKEFELLER PLAZA, NEW YORK, NY, 10112, US

(73) Assignee: Ono Pharmaceutical Co. Ltd., Osaka (JP)

(21) Appl. No.: 11/486,321

(22) Filed: Jul. 12, 2006

Related U.S. Application Data
(62) Division of application No. 10/114,893, filed on Apr. 2, 2002.
Application No. 10/114,893 is a continuation of application No. 09/413,232, filed on Oct. 6, 1999, now abandoned.
Application No. 09/413,232 is a continuation-in-part of application No. 08/833,823, filed on Apr. 10, 1997, now Pat. No. 5,969,093.
Application No. 08/833,823 is a division of application No. 08/514,014, filed on Aug. 11, 1995, now Pat. No. 5,707,829.
Application No. 09/413,232 is a continuation-in-part of application No. 09/189,700, filed on Nov. 10, 1998, now abandoned.
Application No. 09/189,700 is a division of application No. 08/833,823, filed on Apr. 10, 1997, now Pat. No. 5,969,093.
Application No. 08/833,823 is a division of application No. 08/514,014, filed on Aug. 11, 1995, now Pat. No. 5,707,829.
Application No. 09/413,232 is a continuation-in-part of application No. 08/866,022, filed on May 30, 1997, now abandoned.
Application No. 08/866,022 is a division of application No. 08/628,364, filed on Apr. 5, 1996, now abandoned.
Application No. 09/413,232 is a continuation-in-part of application No. 08/924,838, filed on Sep. 5, 1997, now Pat. No. 5,972,652.
Application No. 08/924,838 is a division of application No. 08/628,364, filed on Apr. 5, 1996, now abandoned.
Application No. 09/413,232 is a continuation-in-part of application No. 09/005,986, filed on Jan. 12, 1998, now abandoned.
Application No. 09/005,986 is a continuation-in-part of application No. 08/924,838, filed on Sep. 5, 1997, now Pat. No. 5,972,652.
Application No. 08/924,838 is a division of application No. 08/628,364, filed on Apr. 5, 1996, now abandoned.
Application No. 09/413,232 is a continuation-in-part of application No. 09/203,988, filed on Dec. 2, 1998, now abandoned.
Application No. 09/203,988 is a division of application No. 08/783,395, filed on Jan. 13, 1997, now Pat. No. 5,945,302.
Application No. 08/783,395 is a continuation-in-part of application No. 08/628,364, filed on Apr. 5, 1996, now abandoned.
Application No. 09/413,232 is a continuation-in-part of application No. 08/635,311, filed on Apr. 19, 1996, now abandoned.
Application No. 09/413,232 is a continuation-in-part of application No. 08/781,226, filed on Jan. 10, 1997, now abandoned.
Application No. 08/781,226 is a continuation-in-part of application No. 08/635,311, filed on Apr. 19, 1996, now abandoned.
Application No. 09/413,232 is a continuation-in-part of application No. 08/659,224, filed on Jun. 7, 1996, now abandoned.
Application No. 09/413,232 is a continuation-in-part of application No. 08/867,677, filed on Jun. 2, 1997, now abandoned.
Application No. 08/867,677 is a continuation-in-part of application No. 08/659,224, filed on Jun. 7, 1996, now abandoned.
Application No. 09/413,232 is a continuation-in-part of application No. 09/152,600, filed on Sep. 14, 1998, now abandoned.
Application No. 09/152,600 is a division of application No. 08/664,596, filed on Jun. 17, 1996, now Pat. No. 5,807,703.
Application No. 09/413,232 is a continuation-in-part of application No. 09/153,264, filed on Sep. 14, 1998, now abandoned.
Application No. 09/153,264 is a division of application No. 08/739,775, filed on Oct. 30, 1996, now Pat. No. 5,837,490.
Application No. 08/739,775 is a continuation-in-part of application No. 08/721,923, filed on Sep. 27, 1996, now abandoned.
Application No. 08/721,923 is a continuation-in-part of application No. 08/664,596, filed on Jun. 17, 1996, now Pat. No. 5,807,703.
Application No. 09/413,232 is a continuation-in-part of application No. 09/152,426, filed on Sep. 14, 1998, now abandoned.
Application No. 09/152,426 is a division of application No. 08/738,367, filed on Oct. 25, 1996, now Pat. No. 5,827,688.
Application No. 08/738,367 is a continuation-in-part of application No. 08/721,926, filed on Sep. 27, 1996, now abandoned.
Application No. 08/721,926 is a continuation-in-part of application No. 08/664,596, filed on Jun. 17, 1996, now Pat. No. 5,807,703.
Application No. 09/413,232 is a continuation-in-part of application No. 09/152,914, filed on Sep. 14, 1998, now abandoned.
Application No. 09/152,914 is a division of application No. 08/721,798, filed on Sep. 27, 1996, now Pat. No. 5,807,709.
Application No. 08/721,798 is a continuation-in-part of application No. 08/664,596, filed on Jun. 17, 1996, now Pat. No. 5,807,703.
Application No. 09/413,232 is a continuation-in-part of application No. 08/721,488, filed on Sep. 27, 1996, now Pat. No. 5,965,388.
Application No. 08/721,488 is a continuation-in-part of application No. 08/677,231, filed on Jul. 9, 1996, now abandoned.
Application No. 09/413,232 is a continuation-in-part of application No. 08/955,557, filed on Oct. 18, 1997, now abandoned.
Application No. 08/955,557 is a continuation-in-part of application No. 08/677,231, filed on Jul. 9, 1996, now abandoned.
Application No. 08/955,557 is a continuation-in-part of application No. 08/701,819, filed on Aug. 23, 1996, now abandoned.
Application No. 08/701,819 is a continuation-in-part of application No. 08/677,231, filed on Jul. 9, 1996, now abandoned.
Application No. 08/955,557 is a continuation-in-part of application No. 08/721,488, filed on Sep. 27, 1996, now Pat. No. 5,965,388.
Application No. 08/721,488 is a continuation-in-part of application No. 08/677,231, filed on Jul. 9, 1996, now abandoned.
Application No. 08/955,557 is a continuation-in-part of application No. 08/739,066, filed on Oct. 28, 1996, now abandoned.
Application No. 08/739,066 is a continuation-in-part of application No. 08/721,488, filed on Sep. 27, 1996, now Pat. No. 5,965,388.
Application No. 08/721,488 is a continuation-in-part of application No. 08/677,231, filed on Jul. 9, 1996, now abandoned.
Application No. 09/413,232 is a continuation-in-part of application No. 08/887,029, filed on Jul. 2, 1997, now abandoned.
Application No. 08/887,029 is a division of application No. 08/686,878, filed on Jul. 26, 1996, now Pat. No. 5,708,157.
Application No. 09/413,232 is a continuation-in-part of application No. 08/721,924, filed on Sep. 27, 1996, now Pat. No. 5,969,125.
Application No. 08/721,924 is a continuation-in-part of application No. 08/686,878, filed on Jul. 26, 1996, now Pat. No. 5,708,157.
Application No. 09/413,232 is a continuation-in-part of application No. 09/266,148, filed on Mar. 10, 1999, now abandoned.
Application No. 09/266,148 is a division of application No. 08/721,924, filed on Sep. 27, 1996, now Pat. No. 5,969,125.
Application No. 08/721,924 is a continuation-in-part of application No. 08/686,878, filed on Jul. 26, 1996, now Pat. No. 5,708,157.
Application No. 09/413,232 is a continuation-in-part of application No. 08/976,110, filed on Nov. 21, 1997, now abandoned.
Application No. 08/976,110 is a continuation-in-part of application No. 08/686,878, filed on Jul. 26, 1996, now Pat. No. 5,708,157.
Application No. 08/976,110 is a continuation-in-part of application No. 08/702,081, filed on Aug. 23, 1996, now abandoned.
Application No. 08/702,081 is a continuation-in-part of application No. 08/686,878, filed on Jul. 26, 1996, now Pat. No. 5,708,157.
Application No. 08/976,110 is a continuation-in-part of application No. 08/721,489, filed on Sep. 27, 1996, now Pat. No. 5,786,465.
Application No. 08/721,489 is a continuation-in-part of application No. 08/686,878, filed on Jul. 26, 1996, now Pat. No. 5,708,157.
Application No. 08/976,110 is a continuation-in-part of application No. 08/721,924, filed on Sep. 27, 1996, now Pat. No. 5,969,125.
Application No. 08/721,924 is a continuation-in-part of application No. 08/686,878, filed on Jul. 26, 1996, now Pat. No. 5,708,157.
Application No. 09/413,232 is a continuation-in-part of application No. 08/976,111, filed on Nov. 21, 1997, now abandoned.
Application No. 08/976,111 is a continuation-in-part of application No. 08/691,641, filed on Aug. 2, 1996, now Pat. No. 5,728,819.
Application No. 08/976,111 is a continuation-in-part of application No. 08/702,297, filed on Aug. 23, 1996, now abandoned.
Application No. 08/702,297 is a continuation-in-part of application No. 08/691,641, filed on Aug. 2, 1996, now Pat. No. 5,728,819.
Application No. 09/413,232 is a continuation-in-part of application No. 09/208,181, filed on Dec. 9, 1998, now abandoned.
Application No. 09/208,181 is a division of application No. 08/743,690, filed on Nov. 6, 1996, now abandoned.
Application No. 08/743,690 is a continuation-in-part of application No. 08/702,420, filed on Aug. 14, 1996, now abandoned.
Application No. 09/413,232 is a continuation-in-part of application No. 09/179,034, filed on Oct. 26, 1998, now abandoned.
Application No. 09/179,034 is a division of application No. 08/701,931, filed on Aug. 23, 1996, now abandoned.
Application No. 08/701,931 is a continuation-in-part of application No. 08/702,420, filed on Aug. 14, 1996, now abandoned.
Application No. 09/413,232 is a continuation-in-part of application No. 08/721,925, filed on Sep. 27, 1996, now Pat. No. 5,985,602.
Application No. 08/721,925 is a continuation-in-part of application No. 08/701,931, filed on Aug. 23, 1996, now abandoned.
Application No. 08/701,931 is a continuation-in-part of application No. 08/702,420, filed on Aug. 14, 1996, now abandoned.
Application No. 09/413,232 is a continuation-in-part of application No. 08/975,936, filed on Nov. 21, 1997, now abandoned.
Application No. 08/975,936 is a continuation-in-part of application No. 08/743,690, filed on Nov. 6, 1996, now abandoned.
Application No. 08/743,690 is a continuation-in-part of application No. 08/702,420, filed on Aug. 14, 1996, now abandoned.
Application No. 08/975,936 is a continuation-in-part of application No. 08/701,931, filed on Aug. 23, 1996, now abandoned.
Application No. 08/701,931 is a continuation-in-part of application No. 08/702,420, filed on Aug. 14, 1996, now abandoned.
Application No. 08/975,936 is a continuation-in-part of application No. 08/721,925, filed on Sep. 27, 1996, now Pat. No. 5,985,602.
Application No. 08/721,925 is a continuation-in-part of application No. 08/701,931, filed on Aug. 23, 1996, now abandoned.
Application No. 08/701,931 is a continuation-in-part of application No. 08/702,420, filed on Aug. 14, 1996, now abandoned.
Application No. 09/413,232 is a continuation-in-part of application No. 08/976,112, filed on Nov. 21, 1997, now abandoned.
Application No. 08/976,112 is a continuation-in-part of application No. 08/702,344, filed on Aug. 23, 1996, now Pat. No. 5,723,315.
Application No. 09/413,232 is a continuation-in-part of application No. 09/196,027, filed on Nov. 19, 1998, now abandoned.
Application No. 09/196,027 is a continuation-in-part of application No. 08/976,112, filed on Nov. 21, 1997, now abandoned.
Application No. 08/976,112 is a continuation-in-part of application No. 08/702,344, filed on Aug. 23, 1996, now Pat. No. 5,723,315.
Application No. 09/413,232 is a continuation-in-part of application No. 08/858,830, filed on May 19, 1997, now Pat. No. 5,965,693.
Application No. 08/858,830 is a division of application No. 08/702,080, filed on Aug. 23, 1996, now Pat. No. 5,654,173.
Application No. 09/413,232 is a continuation-in-part of application No. 08/858,834, filed on May 19, 1997, now Pat. No. 5,981,222.
Application No. 08/858,834 is a division of application No. 08/702,080, filed on Aug. 23, 1996, now Pat. No. 5,654,173.
Application No. 09/413,232 is a continuation-in-part of application No. 09/080,695, filed on May 18, 1998, now abandoned.
Application No. 09/080,695 is a continuation-in-part of application No. 08/858,834, filed on May 19, 1997, now Pat. No. 5,981,222.
Application No. 08/858,834 is a division of application No. 08/702,080, filed on Aug. 23, 1996, now Pat. No. 5,654,173.
Application No. 09/413,232 is a continuation-in-part of application No. 09/292,550, filed on Apr. 15, 1999, now abandoned.
Application No. 09/292,550 is a continuation-in-part of application No. 08/858,830, filed on May 19, 1997, now Pat. No. 5,965,693.
Application No. 08/858,830 is a division of application No. 08/702,080, filed on Aug. 23, 1996, now Pat. No. 5,654,173.
Application No. 09/292,550 is a continuation-in-part of application No. 08/858,834, filed on May 19, 1997, now Pat. No. 5,981,222.
Application No. 08/858,834 is a division of application No. 08/702,080, filed on Aug. 23, 1996, now Pat. No. 5,654,173.
Application No. 09/292,550 is a continuation-in-part of application No. 09/080,695, filed on May 18, 1998, now abandoned.
Application No. 09/080,695 is a continuation-in-part of application No. 08/858,834, filed on May 19, 1997, now Pat. No. 5,981,222.
Application No. 08/858,834 is a division of application No. 08/702,080, filed on Aug. 23, 1996, now Pat. No. 5,654,173.
Application No. 09/292,550 is a continuation-in-part of application No. 08/858,832, filed on May 19, 1997, now abandoned.
Application No. 08/858,832 is a division of application No. 08/702,080, filed on Aug. 23, 1996, now Pat. No. 5,654,173.

(51) Int. Cl. C12Q 1/68; C07H 21/04; C12P 21/06; C07K 14/47

(52) U.S. Cl. 435/6; 530/350; 435/69.1; 435/320.1; 435/325; 536/23.5

(57) ABSTRACT

Novel polynucleotides and the proteins encoded thereby are disclosed.

This application is a continuation-in-part of the following applications:
(1) Ser. No. 08/833,823 (GI 6000-DIV), filed Apr. 10, 1997, which is a divisional of Ser. No. 08/514,014 (GI 6000), filed Aug. 11, 1995 and issued as U.S. Pat. No. 5,707,829 on Jan. 13, 1998;
(2) Ser. No. 09/189,700 (GI 6000-DIV2), filed Nov. 10, 1998, which is a divisional of Ser. No. 08/833,823 (GI 6000-DIV), filed Apr. 10, 1997, which is a divisional of Ser. No. 08/514,014 (GI 6000), filed Aug. 11, 1995 and issued as U.S. Pat. No. 5,707,829 on Jan. 13, 1998;
(3) Ser. No. 08/866,022 (GI 6001.B121), filed May 30, 1997, which is a divisional of application Ser. No. 08/628,364 (GI 6001), filed Apr. 5, 1996, now abandoned;
(4) Ser. No. 08/924,838 (GI 6001.B196), filed Sep. 5, 1997, which is a divisional of application Ser. No. 08/628,364 (GI 6001), filed Apr. 5, 1996, now abandoned;
(5) Ser. No. 09/005,986 (GI 6001.B196A), filed Jan. 12, 1998, which is a continuation-in-part of application Ser. No. 08/924,838 (GI 6001.B196), filed Sep. 5, 1997, which is a divisional of application Ser. No. 08/628,364 (GI 6001), filed Apr. 5, 1996, now abandoned;
(6) Ser. No. 09/203,988 (GI 6001.D157-DIV), filed Dec. 2, 1998, which is a divisional of application Ser. No. 08/783,395 (GI 6001.D157), filed Jan. 13, 1997 and issued as U.S. Pat. No. 5,945,302 on Aug. 31, 1999, which was a continuation-in-part of application Ser. No. 08/628,364 (GI 6001), filed Apr. 5, 1996, now abandoned;
(7) Ser. No. 08/635,311 (GI 6002), filed Apr. 19, 1996;
(8) Ser. No. 08/781,226 (GI 6002A), filed Jan. 10, 1997, which is a continuation-in-part of application Ser. No. 08/635,311 (GI 6002), filed Apr. 19, 1996;
(9) Ser. No. 08/659,224 (GI 6003), filed Jun. 7, 1996;
(10) Ser. No. 08/867,677 (GI 6003.D147), filed Jun. 2, 1997, which is a continuation-in-part of application Ser. No. 08/659,224 (GI 6003), filed Jun. 7, 1996;
(11) Ser. No. 09/152,600 (GI 6004-DIV), filed Sep. 14, 1998, which is a divisional of application Ser. No. 08/664,596 (GI 6004), filed Jun. 17, 1996 and issued as U.S. Pat. No. 5,807,703 on Sep. 15, 1998;
(12) Ser. No. 09/153,264 (GI 6004.AS152A-DIV), filed Sep. 14, 1998, which is a divisional of application Ser. No. 08/739,775 (GI 6004.AS152A), filed Oct. 30, 1996 and issued as U.S. Pat. No. 5,837,490 on Nov. 17, 1998, which was a continuation-in-part of application Ser. No. 08/721,923 (GI 6004.AS152), filed Sep. 27, 1996, now abandoned, which was a continuation-in-part of application Ser. No. 08/664,596 (GI 6004), filed Jun. 17, 1996 and issued as U.S. Pat. No. 5,807,703 on Sep. 15, 1998;
(13) Ser. No. 09/152,426 (GI 6004.C195A-DIV), filed Sep. 14, 1998, which is a divisional of application Ser. No. 08/738,367 (GI 6004.C195A), filed Oct. 25, 1996 and issued as U.S. Pat. No. 5,827,688 on Oct. 27, 1998, which was a continuation-in-part of application Ser. No. 08/721,926 (GI 6004.C195), filed Sep. 27, 1996, now abandoned, which was a continuation-in-part of application Ser. No. 08/664,596 (GI 6004), filed Jun. 17, 1996 and issued as U.S. Pat. No. 5,807,703 on Sep. 15, 1998;
(14) Ser. No. 09/152,914 (GI 6004.0276-D), filed Sep. 14, 1998, which is a divisional of application Ser. No. 08/721,798 (GI 6004.O276), filed Sep. 27, 1996 and issued as U.S. Pat. No. 5,807,709 on Sep. 15, 1998, which was a continuation-in-part of application Ser. No. 08/664,596 (GI 6004), filed Jun. 17, 1996 and issued as U.S. Pat. No. 5,807,703 on Sep. 15, 1998;
(15) Ser. No. 08/721,488 (GI 6005A), filed Sep. 27, 1996, which is a continuation-in-part of application Ser. No. 08/677,231 (GI 6005), filed Jul. 9, 1996, now abandoned;
(16) Ser. No. 08/955,557 (GI 6005B), filed Oct. 18, 1997, which is a continuation-in-part of the following applications: (A) application Ser. No. 08/677,231 (GI 6005), filed Jul. 9, 1996, now abandoned; (B) application Ser. No. 08/701,819 (GI 6005.BM46), filed Aug. 23, 1996, now abandoned, which was a continuation-in-part of application Ser. No. 08/677,231 (GI 6005), filed Jul. 9, 1996, now abandoned; (C) application Ser. No. 08/721,488 (GI 6005A), filed Sep. 27, 1996, which is a continuation-in-part of application Ser. No. 08/677,231 (GI 6005), filed Jul. 9, 1996, now abandoned; and (D) application Ser. No. 08/739,066 (GI 6005A.AX56), filed Oct. 28, 1996, now abandoned, which was a continuation-in-part of application Ser. No. 08/721,488 (GI 6005A), filed Sep. 27, 1996, which is a continuation-in-part of application Ser. No. 08/677,231 (GI 6005), filed Jul. 9, 1996, now abandoned;
(17) Ser. No. 08/887,029 (GI 6006-DIV), filed Jul. 2, 1997, which is a divisional of application Ser. No. 08/686,878 (GI 6006), filed Jul. 26, 1996 and issued as U.S. Pat. No. 5,708,157 on Jan. 13, 1998;
(18) Ser. No. 08/721,924 (GI 6006.AP224), filed Sep. 27, 1996, which is a continuation-in-part of application Ser. No. 08/686,878 (GI 6006), filed Jul. 26, 1996 and issued as U.S. Pat. No. 5,708,157 on Jan. 13, 1998;
(19) Ser. No. 09/266,148 (GI 6006.AP224-DIV), filed Mar. 10, 1999, which is a divisional of application Ser. No. 08/721,924 (GI 6006.AP224), filed Sep. 27, 1996, which is a continuation-in-part of application Ser. No. 08/686,878 (GI 6006), filed Jul. 26, 1996 and issued as U.S. Pat. No. 5,708,157 on Jan. 13, 1998;
(20) Ser. No. 08/976,110 (GI 6006B), filed Nov. 21, 1997, which is a continuation-in-part of the following applications: (A) application Ser. No. 08/686,878 (GI 6006), filed Jul. 26, 1996 and issued as U.S. Pat. No. 5,708,157 on Jan. 13, 1998; (B) application Ser. No. 08/702,081 (GI 6006A), filed Aug. 23, 1996, now abandoned, which was a continuation-in-part of application Ser. No. 08/686,878 (GI 6006), filed Jul. 26, 1996 and issued as U.S. Pat. No. 5,708,157 on Jan. 13, 1998; (C) application Ser. No. 08/721,489 (GI 6006.AJ172), filed Sep. 27, 1996 and issued as U.S. Pat. No. 5,786,465 on Jul. 28, 1998, which was a continuation-in-part of application Ser. No. 08/686,878 (GI 6006), filed Jul. 26, 1996 and issued as U.S. Pat. No. 5,708,157 on Jan. 13, 1998; and (D) application Ser. No. 08/721,924 (GI 6006.AP224), filed Sep. 27, 1996, which is a continuation-in-part of application Ser. No. 08/686,878 (GI 6006), filed Jul. 26, 1996 and issued as U.S. Pat. No. 5,708,157 on Jan. 13, 1998;
(21) Ser. No. 08/976,111 (GI 6007A), filed Nov. 21, 1997, which is a continuation-in-part of the following applications: (A) application Ser. No. 08/691,641 (GI 6007), filed Aug. 2, 1996 and issued as U.S. Pat. No. 5,728,819 on Mar. 17, 1998; and (B) application Ser. No. 08/702,297 (GI 6007.BL15), filed Aug. 23, 1996, now abandoned, which was a continuation-in-part of application Ser. No. 08/691,641 (GI 6007), filed Aug. 2, 1996 and issued as U.S. Pat. No. 5,728,819 on Mar. 17, 1998;
(22) Ser. No. 09/208,181 (GI 6008.H438-DIV), filed Dec. 9, 1998, which is a divisional of application Ser. No. 08/743,690 (GI 6008.H438), filed Nov. 6, 1996, now abandoned, which was a continuation-in-part of application Ser. No. 08/702,420 (GI 6008), filed Aug. 14, 1996, now abandoned;
(23) Ser. No. 09/179,034 (GI 6008A-DIV), filed Oct. 26, 1998, which is a divisional of application Ser. No. 08/701,931 (GI 6008A), filed Aug. 23, 1996, now abandoned, which was a continuation-in-part of application Ser. No. 08/702,420 (GI 6008), filed Aug. 14, 1996, now abandoned;
(24) Ser. No. 08/721,925 (GI 6008A.BL205), filed Sep. 27, 1996, which is a continuation-in-part of application Ser. No. 08/701,931 (GI 6008A), filed Aug. 23, 1996, now abandoned, which was a continuation-in-part of application Ser. No. 08/702,420 (GI 6008), filed Aug. 14, 1996, now abandoned;
(25) Ser. No. 08/975,936 (GI 6008B), filed Nov. 21, 1997, which is a continuation-in-part of the following applications: (A) application Ser. No. 08/743,690 (GI 6008.H438), filed Nov. 6, 1996, now abandoned, which was a continuation-in-part of application Ser. No. 08/702,420 (GI 6008), filed Aug. 14, 1996, now abandoned; (B) application Ser. No. 08/701,931 (GI 6008A), filed Aug. 23, 1996, now abandoned, which was a continuation-in-part of application Ser. No. 08/702,420 (GI 6008), filed Aug. 14, 1996, now abandoned; and (C) application Ser. No. 08/721,925 (GI 6008A.BL205); filed Sep. 27, 1996, which is a continuation-in-part of application Ser. No. 08/701,931 (GI 6008A), filed Aug. 23, 1996, now abandoned, which was a continuation-in-part of application Ser. No. 08/702,420 (GI 6008), filed Aug. 14, 1996, now abandoned;
(26) Ser. No. 08/976,112 (GI 6009A), filed Nov. 21, 1997, which is a continuation-in-part of application Ser. No. 08/702,344 (GI 6009), filed Aug. 23, 1996 and issued as U.S. Pat. No. 5,723,315 on Mar. 3, 1998;
(27) Ser. No. 09/196,027 (GI 6009A.CC365), filed Nov. 19, 1998, which is a continuation-in-part of application Ser. No. 08/976,112 (GI 6009A), filed Nov. 21, 1997, which is a continuation-in-part of application Ser. No. 08/702,344 (GI 6009), filed Aug. 23, 1996 and issued as U.S. Pat. No. 5,723,315 on Mar. 3, 1998;
(28) Ser. No. 08/858,830 (GI 6010.BD372), filed May 19, 1997, which is a divisional of application Ser. No. 08/702,080 (GI 6010), filed Aug. 23, 1996 and issued as U.S. Pat. No. 5,654,173 on Aug. 5, 1997;
(29) Ser. No. 08/858,834 (GI 6010.BR533), filed May 19, 1997, which is a divisional of application Ser. No. 08/702,080 (GI 6010), filed Aug. 23, 1996 and issued as U.S. Pat. No. 5,654,173 on Aug. 5, 1997;
(30) Ser. No. 09/080,695 (GI 6010.BR533A), filed May 18, 1998, which is a continuation-in-part of application Ser. No. 08/858,834 (GI 6010.BR533), filed May 19, 1997, which is a divisional of application Ser. No. 08/702,080 (GI 6010), filed Aug. 23, 1996 and issued as U.S. Pat. No. 5,654,173 on Aug. 5, 1997;
(31) Ser. No. 09/292,550 (GI 6010A), filed Apr. 15, 1999, which is a continuation-in-part of the following applications: (A) application Ser. No. 08/858,830 (GI 6010.BD372), filed May 19, 1997, which is a divisional of application Ser. No. 08/702,080 (GI 6010), filed Aug. 23, 1996 and issued as U.S. Pat. No. 5,654,173 on Aug. 5, 1997; (B) application Ser. No. 08/858,834 (GI 6010.BR533), filed May 19, 1997, which is a divisional of application Ser. No. 08/702,080 (GI 6010), filed Aug. 23, 1996 and issued as U.S. Pat. No. 5,654,173 on Aug. 5, 1997; (C) application Ser. No. 09/080,695 (GI 6010.BR533A), filed May 18, 1998, which is a continuation-in-part of application Ser. No. 08/858,834 (GI 6010.BR533), filed May 19, 1997, which is a divisional of application Ser. No. 08/702,080 (GI 6010), filed Aug. 23, 1996 and issued as U.S. Pat. No. 5,654,173 on Aug. 5, 1997; and (D) application Ser. No. 08/858,832 (GI 6010.CC288), filed May 19, 1997, now abandoned, which was a divisional application of Ser. No. 08/702,080 (GI 6010), filed Aug. 23, 1996 and issued as U.S. Pat. No. 5,654,173 on Aug. 5, 1997;
   all of which are incorporated by reference herein.

### FIELD OF THE INVENTION

The present invention provides novel polynucleotides and proteins encoded by such polynucleotides, along with therapeutic, diagnostic and research utilities for these polynucleotides and proteins.

### BACKGROUND OF THE INVENTION

Technology aimed at the discovery of protein factors (including e.g., cytokines, such as lymphokines, interferons, CSFs and interleukins) has matured rapidly over the past decade. The now routine hybridization cloning and expression cloning techniques clone novel polynucleotides “directly” in the sense that they rely on information directly related to the discovered protein (i.e., partial DNA/amino acid sequence of the protein in the case of hybridization cloning; activity of the protein in the case of expression cloning). More recent “indirect” cloning techniques such as signal sequence cloning, which isolates DNA sequences based on the presence of a now well-recognized secretory leader sequence motif, as well as various PCR-based or low stringency hybridization cloning techniques, have advanced the state of the art by making available large numbers of DNA/amino acid sequences for proteins that are known to have biological activity by virtue of their secreted nature in the case of leader sequence cloning, or by virtue of the cell or tissue source in the case of PCR-based techniques. It is to these proteins and the polynucleotides encoding them that the present invention is directed.

### SUMMARY OF THE INVENTION

In one embodiment, the present invention provides a composition comprising an isolated polynucleotide selected from the group consisting of:
(a) a polynucleotide comprising the nucleotide sequence of SEQ ID NO:1;
(b) a polynucleotide comprising the nucleotide sequence of SEQ ID NO:1 from nucleotide 55 to nucleotide 546;
(c) a polynucleotide comprising the nucleotide sequence of SEQ ID NO:1 from nucleotide 145 to nucleotide 546;
(d) a polynucleotide comprising the nucleotide sequence of the full-length protein coding sequence of clone B18_{—}11 deposited with the ATCC under accession number 69868;
(e) a polynucleotide encoding the full-length protein encoded by the cDNA insert of clone B18_{—}11 deposited with the ATCC under accession number 69868;
(f) a polynucleotide comprising the nucleotide sequence of a mature protein coding sequence of clone B18_{—}11 deposited with the ATCC under accession number 69868;
(g) a polynucleotide encoding a mature protein encoded by the cDNA insert of clone B18_{—}11 deposited with the ATCC under accession number 69868;
(h) a polynucleotide encoding a protein comprising the amino acid sequence of SEQ ID NO:2;
(i) a polynucleotide encoding a protein comprising a fragment of the amino acid sequence of SEQ ID NO:2 having biological activity, the fragment comprising eight contiguous amino acids of SEQ ID NO:2;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:1.

Preferably, such polynucleotide comprises the nucleotide sequence of SEQ ID NO:1 from nucleotide 55 to nucleotide 546; the nucleotide sequence of SEQ ID NO:1 from nucleotide 145 to nucleotide 546; the nucleotide sequence of the full-length protein coding sequence of clone B18_{—}11 deposited with the ATCC under accession number 69868; or the nucleotide sequence of a mature protein coding sequence of clone B18_{—}11 deposited with the ATCC under accession number 69868. In other preferred embodiments, the polynucleotide encodes the full-length or a mature protein encoded by the cDNA insert of clone B18_{—}11 deposited with the ATCC under accession number 69868. In yet other preferred embodiments, the present invention provides a polynucleotide encoding a protein comprising the amino acid sequence of SEQ ID NO:2 from amino acid 11 to amino acid 130. In further preferred embodiments, the present invention provides a polynucleotide encoding a protein comprising a fragment of the amino acid sequence of SEQ ID NO:2 having biological activity, the fragment preferably comprising eight (more preferably twenty, most preferably thirty) contiguous amino acids of SEQ ID NO:2, or a polynucleotide encoding a protein comprising a fragment of the amino acid sequence of SEQ ID NO:2 having biological activity, the fragment comprising the amino acid sequence from amino acid 76 to amino acid 85 of SEQ ID NO:2.

Other embodiments provide the gene corresponding to the cDNA sequence of SEQ ID NO:1.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:1, but excluding the poly(A) tail at the 3′ end of SEQ ID NO:1; and
      (ab) the nucleotide sequence of the cDNA insert of clone B18_{—}11 deposited with the ATCC under accession number 69868;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:1, but excluding the poly(A) tail at the 3′ end of SEQ ID NO:1; and
      (bb) the nucleotide sequence of the cDNA insert of clone B18_{—}11 deposited with the ATCC under accession number 69868;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:1, and extending contiguously from a nucleotide sequence corresponding to the 5′ end of SEQ ID NO:1 to a nucleotide sequence corresponding to the 3′ end of SEQ ID NO:1, but excluding the poly(A) tail at the 3′ end of SEQ ID NO:1. Also preferably the polynucleotide isolated according to the above process comprises a nucleotide sequence corresponding to the cDNA sequence of SEQ ID NO:1 from nucleotide 55 to nucleotide 546, and extending contiguously from a nucleotide sequence corresponding to the 5′ end of said sequence of SEQ ID NO:1 from nucleotide 55 to nucleotide 546, to a nucleotide sequence corresponding to the 3′ end of said sequence of SEQ ID NO:1 from nucleotide 55 to nucleotide 546. Also preferably the polynucleotide isolated according to the above process comprises a nucleotide sequence corresponding to the cDNA sequence of SEQ ID NO:1 from nucleotide 145 to nucleotide 546, and extending contiguously from a nucleotide sequence corresponding to the 5′ end of said sequence of SEQ ID NO:1 from nucleotide 145 to nucleotide 546, to a nucleotide sequence corresponding to the 3′ end of said sequence of SEQ ID NO:1 from nucleotide 145 to nucleotide 546.

In other embodiments, the present invention provides a composition comprising a protein, wherein said protein comprises an amino acid sequence selected from the group consisting of:
(a) the amino acid sequence of SEQ ID NO:2;
(b) the amino acid sequence of SEQ ID NO:2 from amino acid 11 to amino acid 130;
(c) a fragment of the amino acid sequence of SEQ ID NO:2, the fragment comprising eight contiguous amino acids of SEQ ID NO:2; and
(d) the amino acid sequence encoded by the cDNA insert of clone B18_{—}11 deposited with the ATCC under accession number 69868;
   the protein being substantially free from other mammalian proteins. Preferably such protein comprises the amino acid sequence of SEQ ID NO:2 or the amino acid sequence of SEQ ID NO:2 from amino acid 11 to amino acid 130. In further preferred embodiments, the present invention provides a protein comprising a fragment of the amino acid sequence of SEQ ID NO:2 having biological activity, the fragment preferably comprising eight (more preferably twenty, most preferably thirty) contiguous amino acids of SEQ ID NO:2, or a protein comprising a fragment of the amino acid sequence of SEQ ID NO:2 having biological activity, the fragment comprising the amino acid sequence from amino acid 76 to amino acid 85 of SEQ ID NO:2.

In one embodiment, the present invention provides a composition comprising an isolated polynucleotide selected from the group consisting of:
(a) a polynucleotide comprising the nucleotide sequence of SEQ ID NO:3;
(b) a polynucleotide comprising the nucleotide sequence of SEQ ID NO:3 from nucleotide 67 to nucleotide 348;
(c) a polynucleotide comprising the nucleotide sequence of SEQ ID NO:3 from nucleotide 130 to nucleotide 348;
(d) a polynucleotide comprising the nucleotide sequence of the full-length protein coding sequence of clone H174_{—}10 deposited with the ATCC under accession number 69882;
(e) a polynucleotide encoding the full-length protein encoded by the cDNA insert of clone H174_{—}10 deposited with the ATCC under accession number 69882;
(f) a polynucleotide comprising the nucleotide sequence of a mature protein coding sequence of clone H174_{—}10 deposited with the ATCC under accession number 69882;
(g) a polynucleotide encoding a mature protein encoded by the cDNA insert of clone H174_{—}10 deposited with the ATCC under accession number 69882;
(h) a polynucleotide encoding a protein comprising the amino acid sequence of SEQ ID NO:4;
(i) a polynucleotide encoding a protein comprising a fragment of the amino acid sequence of SEQ ID NO:4 having biological activity, the fragment comprising eight contiguous amino acids of SEQ ID NO:4;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:3.

Preferably, such polynucleotide comprises the nucleotide sequence of SEQ ID NO:3 from nucleotide 67 to nucleotide 348; the nucleotide sequence of SEQ ID NO:3 from nucleotide 130 to nucleotide 348; the nucleotide sequence of the full-length protein coding sequence of clone H174_{—}10 deposited with the ATCC under accession number 69882; or the nucleotide sequence of a mature protein coding sequence of clone H174_{—}10 deposited with the ATCC under accession number 69882. In other preferred embodiments, the polynucleotide encodes the full-length or a mature protein encoded by the cDNA insert of clone H174_{—}10 deposited with the ATCC under accession number 69882. In further preferred embodiments, the present invention provides a polynucleotide encoding a protein comprising a fragment of the amino acid sequence of SEQ ID NO:4 having biological activity, the fragment preferably comprising eight (more preferably twenty, most preferably thirty) contiguous amino acids of SEQ ID NO:4, or a polynucleotide encoding a protein comprising a fragment of the amino acid sequence of SEQ ID NO:4 having biological activity, the fragment comprising the amino acid sequence from amino acid 42 to amino acid 51 of SEQ ID NO:4.

Other embodiments provide the gene corresponding to the cDNA sequence of SEQ ID NO:3.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:3, but excluding the poly(A) tail at the 3′ end of SEQ ID NO:3; and
      (ab) the nucleotide sequence of the cDNA insert of clone H174_{—}10 deposited with the ATCC under accession number 69882;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:3, but excluding the poly(A) tail at the 3′ end of SEQ ID NO:3; and
      (bb) the nucleotide sequence of the cDNA insert of clone H174_{—}10 deposited with the ATCC under accession number 69882;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:3, and extending contiguously from a nucleotide sequence corresponding to the 5′ end of SEQ ID NO:3 to a nucleotide sequence corresponding to the 3′ end of SEQ ID NO:3, but excluding the poly(A) tail at the 3′ end of SEQ ID NO:3. Also preferably the polynucleotide isolated according to the above process comprises a nucleotide sequence corresponding to the cDNA sequence of SEQ ID NO:3 from nucleotide 67 to nucleotide 348, and extending contiguously from a nucleotide sequence corresponding to the 5′ end of said sequence of SEQ ID NO:3 from nucleotide 67 to nucleotide 348, to a nucleotide sequence corresponding to the 3′ end of said sequence of SEQ ID NO:3 from nucleotide 67 to nucleotide 348. Also preferably the polynucleotide isolated according to the above process comprises a nucleotide sequence corresponding to the cDNA sequence of SEQ ID NO:3 from nucleotide 130 to nucleotide 348, and extending contiguously from a nucleotide sequence corresponding to the 5′ end of said sequence of SEQ ID NO:3 from nucleotide 130 to nucleotide 348, to a nucleotide sequence corresponding to the 3′ end of said sequence of SEQ ID NO:3 from nucleotide 130 to nucleotide 348.

In other embodiments, the present invention provides a composition comprising a protein, wherein said protein comprises an amino acid sequence selected from the group consisting of:
(a) the amino acid sequence of SEQ ID NO:4;
(b) a fragment of the amino acid sequence of SEQ ID NO:4, the fragment comprising eight contiguous amino acids of SEQ ID NO:4; and
(c) the amino acid sequence encoded by the cDNA insert of clone H174_{—}10 deposited with the ATCC under accession number 69882;
   the protein being substantially free from other mammalian proteins. Preferably such protein comprises the amino acid sequence of SEQ ID NO:4. In further preferred embodiments, the present invention provides a protein comprising a fragment of the amino acid sequence of SEQ ID NO:4 having biological activity, the fragment preferably comprising eight (more preferably twenty, most preferably thirty) contiguous amino acids of SEQ ID NO:4, or a protein comprising a fragment of the amino acid sequence of SEQ ID NO:4 having biological activity, the fragment comprising the amino acid sequence from amino acid 42 to amino acid 51 of SEQ ID NO:4.

In one embodiment, the present invention provides a composition comprising an isolated polynucleotide selected from the group consisting of:
(a) a polynucleotide comprising the nucleotide sequence of SEQ ID NO:5;
(b) a polynucleotide comprising the nucleotide sequence of SEQ ID NO:5 from nucleotide 75 to nucleotide 356;
(c) a polynucleotide comprising the nucleotide sequence of SEQ ID NO:5 from nucleotide 138 to nucleotide 356;
(d) a polynucleotide comprising the nucleotide sequence of the full-length protein coding sequence of clone H174_{—}43 deposited with the ATCC under accession number 69882;
(e) a polynucleotide encoding the full-length protein encoded by the cDNA insert of clone H174_{—}43 deposited with the ATCC under accession number 69882;
(f) a polynucleotide comprising the nucleotide sequence of a mature protein coding sequence of clone H174_{—}43 deposited with the ATCC under accession number 69882;
(g) a polynucleotide encoding a mature protein encoded by the cDNA insert of clone H174_{—}43 deposited with the ATCC under accession number 69882;
(h) a polynucleotide encoding a protein comprising the amino acid sequence of SEQ ID NO:6;
(i) a polynucleotide encoding a protein comprising a fragment of the amino acid sequence of SEQ ID NO:6 having biological activity, the fragment comprising eight contiguous amino acids of SEQ ID NO:6;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:5.

Preferably, such polynucleotide comprises the nucleotide sequence of SEQ ID NO:5 from nucleotide 75 to nucleotide 356; the nucleotide sequence of SEQ ID NO:5 from nucleotide 138 to nucleotide 356; the nucleotide sequence of the full-length protein coding sequence of clone H174_{—}43 deposited with the ATCC under accession number 69882; or the nucleotide sequence of a mature protein coding sequence of clone H174_{—}43 deposited with the ATCC under accession number 69882. In other preferred embodiments, the polynucleotide encodes the full-length or a mature protein encoded by the cDNA insert of clone H174_{—}43 deposited with the ATCC under accession number 69882. In further preferred embodiments, the present invention provides a polynucleotide encoding a protein comprising a fragment of the amino acid sequence of SEQ ID NO:6 having biological activity, the fragment preferably comprising eight (more preferably twenty, most preferably thirty) contiguous amino acids of SEQ ID NO:6, or a polynucleotide encoding a protein comprising a fragment of the amino acid sequence of SEQ ID NO:6 having biological activity, the fragment comprising the amino acid sequence from amino acid 42 to amino acid 51 of SEQ ID NO:6.

Other embodiments provide the gene corresponding to the cDNA sequence of SEQ ID NO:5.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:5; and
      (ab) the nucleotide sequence of the cDNA insert of clone H174_{—}43 deposited with the ATCC under accession number 69882;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:5; and
      (bb) the nucleotide sequence of the cDNA insert of clone H174_{—}43 deposited with the ATCC under accession number 69882;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:5, and extending contiguously from a nucleotide sequence corresponding to the 5′ end of SEQ ID NO:5 to a nucleotide sequence corresponding to the 3′ end of SEQ ID NO:5. Also preferably the polynucleotide isolated according to the above process comprises a nucleotide sequence corresponding to the cDNA sequence of SEQ ID NO:5 from nucleotide 75 to nucleotide 356, and extending contiguously from a nucleotide sequence corresponding to the 5′ end of said sequence of SEQ ID NO:5 from nucleotide 75 to nucleotide 356, to a nucleotide sequence corresponding to the 3′ end of said sequence of SEQ ID NO:5 from nucleotide 75 to nucleotide 356. Also preferably the polynucleotide isolated according to the above process comprises a nucleotide sequence corresponding to the cDNA sequence of SEQ ID NO:5 from nucleotide 138 to nucleotide 356, and extending contiguously from a nucleotide sequence corresponding to the 5′ end of said sequence of SEQ ID NO:5 from nucleotide 138 to nucleotide 356, to a nucleotide sequence corresponding to the 3′ end of said sequence of SEQ ID NO:5 from nucleotide 138 to nucleotide 356.

In other embodiments, the present invention provides a composition comprising a protein, wherein said protein comprises an amino acid sequence selected from the group consisting of:
(a) the amino acid sequence of SEQ ID NO:6;
(b) a fragment of the amino acid sequence of SEQ ID NO:6, the fragment comprising eight contiguous amino acids of SEQ ID NO:6; and
(c) the amino acid sequence encoded by the cDNA insert of clone H174_{—}43 deposited with the ATCC under accession number 69882;
   the protein being substantially free from other mammalian proteins. Preferably such protein comprises the amino acid sequence of SEQ ID NO:6. In further preferred embodiments, the present invention provides a protein comprising a fragment of the amino acid sequence of SEQ ID NO:6 having biological activity, the fragment preferably comprising eight (more preferably twenty, most preferably thirty) contiguous amino acids of SEQ ID NO:6, or a protein comprising a fragment of the amino acid sequence of SEQ ID NO:6 having biological activity, the fragment comprising the amino acid sequence from amino acid 42 to amino acid 51 of SEQ ID NO:6.

In one embodiment, the present invention provides a composition comprising an isolated polynucleotide selected from the group consisting of:
(a) a polynucleotide comprising the nucleotide sequence of SEQ ID NO:7;
(b) a polynucleotide comprising the nucleotide sequence of SEQ ID NO:7 from nucleotide 13 to nucleotide 1422;
(c) a polynucleotide comprising the nucleotide sequence of SEQ ID NO:7 from nucleotide 97 to nucleotide 1422;
(d) a polynucleotide comprising the nucleotide sequence of SEQ ID NO:7 from nucleotide 40 to nucleotide 1456;
(e) a polynucleotide comprising the nucleotide sequence of the full-length protein coding sequence of clone J5_{—}3 deposited with the ATCC under accession number 69885;
(f) a polynucleotide encoding the full-length protein encoded by the cDNA insert of clone J5_{—}3 deposited with the ATCC under accession number 69885;
(g) a polynucleotide comprising the nucleotide sequence of a mature protein coding sequence of clone J5_{—}3 deposited with the ATCC under accession number 69885;
(h) a polynucleotide encoding a mature protein encoded by the cDNA insert of clone J5_{—}3 deposited with the ATCC under accession number 69885;
(i) a polynucleotide encoding a protein comprising the amino acid sequence of SEQ ID NO:8;
(j) a polynucleotide encoding a protein comprising a fragment of the amino acid sequence of SEQ ID NO:8 having biological activity, the fragment comprising eight contiguous amino acids of SEQ ID NO:8;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:7.

Preferably, such polynucleotide comprises the nucleotide sequence of SEQ ID NO:7 from nucleotide 13 to nucleotide 1422; the nucleotide sequence of SEQ ID NO:7 from nucleotide 97 to nucleotide 1422; the nucleotide sequence of SEQ ID NO:7 from nucleotide 40 to nucleotide 1456; the nucleotide sequence of the full-length protein coding sequence of clone J5_{—}3 deposited with the ATCC under accession number 69885; or the nucleotide sequence of a mature protein coding sequence of clone J5_{—}3 deposited with the ATCC under accession number 69885. In other preferred embodiments, the polynucleotide encodes the full-length or a mature protein encoded by the cDNA insert of clone J5_{—}3 deposited with the ATCC under accession number 69885. In further preferred embodiments, the present invention provides a polynucleotide encoding a protein comprising a fragment of the amino acid sequence of SEQ ID NO:8 having biological activity, the fragment preferably comprising eight (more preferably twenty, most preferably thirty) contiguous amino acids of SEQ ID NO:8, or a polynucleotide encoding a protein comprising a fragment of the amino acid sequence of SEQ ID NO:8 having biological activity, the fragment comprising the amino acid sequence from amino acid 230 to amino acid 239 of SEQ ID NO:8.

Other embodiments provide the gene corresponding to the cDNA sequence of SEQ ID NO:7.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:7; and
      (ab) the nucleotide sequence of the cDNA insert of clone J5_{—}3 deposited with the ATCC under accession number 69885;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:7; and
      (bb) the nucleotide sequence of the cDNA insert of clone J5_{—}3 deposited with the ATCC under accession number 69885;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:7, and extending contiguously from a nucleotide sequence corresponding to the 5′ end of SEQ ID NO:7 to a nucleotide sequence corresponding to the 3′ end of SEQ ID NO:7. Also preferably the polynucleotide isolated according to the above process comprises a nucleotide sequence corresponding to the cDNA sequence of SEQ ID NO:7 from nucleotide 13 to nucleotide 1422, and extending contiguously from a nucleotide sequence corresponding to the 5′ end of said sequence of SEQ ID NO:7 from nucleotide 13 to nucleotide 1422, to a nucleotide sequence corresponding to the 3′ end of said sequence of SEQ ID NO:7 from nucleotide 13 to nucleotide 1422. Also preferably the polynucleotide isolated according to the above process comprises a nucleotide sequence corresponding to the cDNA sequence of SEQ ID NO:7 from nucleotide 97 to nucleotide 1422, and extending contiguously from a nucleotide sequence corresponding to the 5′ end of said sequence of SEQ ID NO:7 from nucleotide 97 to nucleotide 1422, to a nucleotide sequence corresponding to the 3′ end of said sequence of SEQ ID NO:7 from nucleotide 97 to nucleotide 1422. Also preferably the polynucleotide isolated according to the above process comprises a nucleotide sequence corresponding to the cDNA sequence of SEQ ID NO:7 from nucleotide 40 to nucleotide 1456, and extending contiguously from a nucleotide sequence corresponding to the 5′ end of said sequence of SEQ ID NO:7 from nucleotide 40 to nucleotide 1456, to a nucleotide sequence corresponding to the 3′ end of said sequence of SEQ ID NO:7 from nucleotide to nucleotide 1456.

In other embodiments, the present invention provides a composition comprising a protein, wherein said protein comprises an amino acid sequence selected from the group consisting of:
(a) the amino acid sequence of SEQ ID NO:8;
(b) a fragment of the amino acid sequence of SEQ ID NO:8, the fragment comprising eight contiguous amino acids of SEQ ID NO:8; and
(c) the amino acid sequence encoded by the cDNA insert of clone J5_{—}3 deposited with the ATCC under accession number 69885;
   the protein being substantially free from other mammalian proteins. Preferably such protein comprises the amino acid sequence of SEQ ID NO:8. In further preferred embodiments, the present invention provides a protein comprising a fragment of the amino acid sequence of SEQ ID NO:8 having biological activity, the fragment preferably comprising eight (more preferably twenty, most preferably thirty) contiguous amino acids of SEQ ID NO:8, or a protein comprising a fragment of the amino acid sequence of SEQ ID NO:8 having biological activity, the fragment comprising the amino acid sequence from amino acid 230 to amino acid 239 of SEQ ID NO:8.

In one embodiment, the present invention provides a composition comprising an isolated polynucleotide selected from the group consisting of:
(a) a polynucleotide comprising the nucleotide sequence of SEQ ID NO:9;
(b) a polynucleotide comprising the nucleotide sequence of SEQ ID NO:9 from nucleotide 24 to nucleotide 2006;
(c) a polynucleotide comprising the nucleotide sequence of SEQ ID NO:9 from nucleotide 1563 to nucleotide 2006;
(d) a polynucleotide comprising the nucleotide sequence of the full-length protein coding sequence of clone J422_{—}1 deposited with the ATCC under accession number 69884;
(e) a polynucleotide encoding the full-length protein encoded by the cDNA insert of clone J422_{—}1 deposited with the ATCC under accession number 69884;
(f) a polynucleotide comprising the nucleotide sequence of a mature protein coding sequence of clone J422_{—}1 deposited with the ATCC under accession number 69884;
(g) a polynucleotide encoding a mature protein encoded by the cDNA insert of clone J422_{—}1 deposited with the ATCC under accession number 69884;
(h) a polynucleotide encoding a protein comprising the amino acid sequence of SEQ ID NO:10;
(i) a polynucleotide encoding a protein comprising a fragment of the amino acid sequence of SEQ ID NO:10 having biological activity, the fragment comprising eight contiguous amino acids of SEQ ID NO:10;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:9.

Preferably, such polynucleotide comprises the nucleotide sequence of SEQ ID NO:9 from nucleotide 24 to nucleotide 2006; the nucleotide sequence of SEQ ID NO:9 from nucleotide 1563 to nucleotide 2006; the nucleotide sequence of the full-length protein coding sequence of clone J422_{—}1 deposited with the ATCC under accession number 69884; or the nucleotide sequence of a mature protein coding sequence of clone J422_{—}1 deposited with the ATCC under accession number 69884. In other preferred embodiments, the polynucleotide encodes the full-length or a mature protein encoded by the cDNA insert of clone J422_{—}1 deposited with the ATCC under accession number 69884. In further preferred embodiments, the present invention provides a polynucleotide encoding a protein comprising a fragment of the amino acid sequence of SEQ ID NO:10 having biological activity, the fragment preferably comprising eight (more preferably twenty, most preferably thirty) contiguous amino acids of SEQ ID NO:10, or a polynucleotide encoding a protein comprising a fragment of the amino acid sequence of SEQ ID NO:10 having biological activity, the fragment comprising the amino acid sequence from amino acid 325 to amino acid 334 of SEQ ID NO:10.

Other embodiments provide the gene corresponding to the cDNA sequence of SEQ ID NO:9.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:9, but excluding the poly(A) tail at the 3′ end of SEQ ID NO:9; and
      (ab) the nucleotide sequence of the cDNA insert of clone J422_{—}1 deposited with the ATCC under accession number 69884;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:9, but excluding the poly(A) tail at the 3′ end of SEQ ID NO:9; and
      (bb) the nucleotide sequence of the cDNA insert of clone J422_{—}1 deposited with the ATCC under accession number 69884;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:9, and extending contiguously from a nucleotide sequence corresponding to the 5′ end of SEQ ID NO:9 to a nucleotide sequence corresponding to the 3′ end of SEQ ID NO:9, but excluding the poly(A) tail at the 3′ end of SEQ ID NO:9. Also preferably the polynucleotide isolated according to the above process comprises a nucleotide sequence corresponding to the cDNA sequence of SEQ ID NO:9 from nucleotide 24 to nucleotide 2006, and extending contiguously from a nucleotide sequence corresponding to the 5′ end of said sequence of SEQ ID NO:9 from nucleotide 24 to nucleotide 2006, to a nucleotide sequence corresponding to the 3′ end of said sequence of SEQ ID NO:9 from nucleotide 24 to nucleotide 2006. Also preferably the polynucleotide isolated according to the above process comprises a nucleotide sequence corresponding to the cDNA sequence of SEQ ID NO:9 from nucleotide 1563 to nucleotide 2006, and extending contiguously from a nucleotide sequence corresponding to the 5′ end of said sequence of SEQ ID NO:9 from nucleotide 1563 to nucleotide 2006, to a nucleotide sequence corresponding to the 3′ end of said sequence of SEQ ID NO:9 from nucleotide 1563 to nucleotide 2006.

In other embodiments, the present invention provides a composition comprising a protein, wherein said protein comprises an amino acid sequence selected from the group consisting of:
(a) the amino acid sequence of SEQ ID NO:10;
(b) a fragment of the amino acid sequence of SEQ ID NO:10, the fragment comprising eight contiguous amino acids of SEQ ID NO:10; and
(c) the amino acid sequence encoded by the cDNA insert of clone J422_{—}1 deposited with the ATCC under accession number 69884;
   the protein being substantially free from other mammalian proteins. Preferably such protein comprises the amino acid sequence of SEQ ID NO:10. In further preferred embodiments, the present invention provides a protein comprising a fragment of the amino acid sequence of SEQ ID NO:10 having biological activity, the fragment preferably comprising eight (more preferably twenty, most preferably thirty) contiguous amino acids of SEQ ID NO:10, or a protein comprising a fragment of the amino acid sequence of SEQ ID NO:10 having biological activity, the fragment comprising the amino acid sequence from amino acid 325 to amino acid 334 of SEQ ID NO:10.

In one embodiment, the present invention provides a composition comprising an isolated polynucleotide selected from the group consisting of:
(a) a polynucleotide comprising the nucleotide sequence of SEQ ID NO:11;
(b) a polynucleotide comprising the nucleotide sequence of SEQ ID NO:11 from nucleotide 48 to nucleotide 446;
(c) a polynucleotide comprising the nucleotide sequence of SEQ ID NO:11 from nucleotide 93 to nucleotide 446;
(d) a polynucleotide comprising the nucleotide sequence of the full-length protein coding sequence of clone L105_{—}74 deposited with the ATCC under accession number 69883;
(e) a polynucleotide encoding the full-length protein encoded by the cDNA insert of clone L105_{—}74 deposited with the ATCC under accession number 69883;
(f) a polynucleotide comprising the nucleotide sequence of a mature protein coding sequence of clone L105_{—}74 deposited with the ATCC under accession number 69883;
(g) a polynucleotide encoding a mature protein encoded by the cDNA insert of clone L105_{—}74 deposited with the ATCC under accession number 69883;
(h) a polynucleotide encoding a protein comprising the amino acid sequence of SEQ ID NO:12;
(i) a polynucleotide encoding a protein comprising a fragment of the amino acid sequence of SEQ ID NO:12 having biological activity, the fragment comprising eight contiguous amino acids of SEQ ID NO:12;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:11.

Preferably, such polynucleotide comprises the nucleotide sequence of SEQ ID NO:11 from nucleotide 48 to nucleotide 446; the nucleotide sequence of SEQ ID NO:11 from nucleotide 93 to nucleotide 446; the nucleotide sequence of the full-length protein coding sequence of clone L105_{—}74 deposited with the ATCC under accession number 69883; or the nucleotide sequence of a mature protein coding sequence of clone L105_{—}74 deposited with the ATCC under accession number 69883. In other preferred embodiments, the polynucleotide encodes the full-length or a mature protein encoded by the cDNA insert of clone L105_{—}74 deposited with the ATCC under accession number 69883. In further preferred embodiments, the present invention provides a polynucleotide encoding a protein comprising a fragment of the amino acid sequence of SEQ ID NO:12 having biological activity, the fragment preferably comprising eight (more preferably twenty, most preferably thirty) contiguous amino acids of SEQ ID NO:12, or a polynucleotide encoding a protein comprising a fragment of the amino acid sequence of SEQ ID NO:12 having biological activity, the fragment comprising the amino acid sequence from amino acid 61 to amino acid 70 of SEQ ID NO:12.

Other embodiments provide the gene corresponding to the cDNA sequence of SEQ ID NO:11.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:11; and
      (ab) the nucleotide sequence of the cDNA insert of clone L105_{—}74 deposited with the ATCC under accession number 69883;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:11; and
      (bb) the nucleotide sequence of the cDNA insert of clone L105_{—}74 deposited with the ATCC under accession number 69883;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:11, and extending contiguously from a nucleotide sequence corresponding to the 5′ end of SEQ ID NO:11 to a nucleotide sequence corresponding to the 3′ end of SEQ ID NO:11. Also preferably the polynucleotide isolated according to the above process comprises a nucleotide sequence corresponding to the cDNA sequence of SEQ ID NO:11 from nucleotide 48 to nucleotide 446, and extending contiguously from a nucleotide sequence corresponding to the 5′ end of said sequence of SEQ ID NO:11 from nucleotide 48 to nucleotide 446, to a nucleotide sequence corresponding to the 3′ end of said sequence of SEQ ID NO:11 from nucleotide 48 to nucleotide 446. Also preferably the polynucleotide isolated according to the above process comprises a nucleotide sequence corresponding to the cDNA sequence of SEQ ID NO:11 from nucleotide 93 to nucleotide 446, and extending contiguously from a nucleotide sequence corresponding to the 5′ end of said sequence of SEQ ID NO:11 from nucleotide 93 to nucleotide 446, to a nucleotide sequence corresponding to the 3′ end of said sequence of SEQ ID NO:11 from nucleotide 93 to nucleotide 446.

In other embodiments, the present invention provides a composition comprising a protein, wherein said protein comprises an amino acid sequence selected from the group consisting of:
(a) the amino acid sequence of SEQ ID NO:12;
(b) a fragment of the amino acid sequence of SEQ ID NO:12, the fragment comprising eight contiguous amino acids of SEQ ID NO:12; and
(c) the amino acid sequence encoded by the cDNA insert of clone L105_{—}74 deposited with the ATCC under accession number 69883;
   the protein being substantially free from other mammalian proteins. Preferably such protein comprises the amino acid sequence of SEQ ID NO:12. In further preferred embodiments, the present invention provides a protein comprising a fragment of the amino acid sequence of SEQ ID NO:12 having biological activity, the fragment preferably comprising eight (more preferably twenty, most preferably thirty) contiguous amino acids of SEQ ID NO:12, or a protein comprising a fragment of the amino acid sequence of SEQ ID NO:12 having biological activity, the fragment comprising the amino acid sequence from amino acid 61 to amino acid 70 of SEQ ID NO:12.

In one embodiment, the present invention provides a composition comprising an isolated polynucleotide selected from the group consisting of:
(a) a polynucleotide comprising the nucleotide sequence of SEQ ID NO:13;
(b) a polynucleotide comprising the nucleotide sequence of SEQ ID NO:13 from nucleotide 370 to nucleotide 1338;
(c) a polynucleotide comprising the nucleotide sequence of SEQ ID NO:13 from nucleotide 700 to nucleotide 1338;
(d) a polynucleotide comprising the nucleotide sequence of SEQ ID NO:13 from nucleotide 621 to nucleotide 1474;
(e) a polynucleotide comprising the nucleotide sequence of the full-length protein coding sequence of clone B121_{—}1 deposited with the ATCC under accession number 98019;
(f) a polynucleotide encoding the full-length protein encoded by the cDNA insert of clone B121_{—}1 deposited with the ATCC under accession number 98019;
(g) a polynucleotide comprising the nucleotide sequence of a mature protein coding sequence of clone B121_{—}1 deposited with the ATCC under accession number 98019;
(h) a polynucleotide encoding a mature protein encoded by the cDNA insert of clone B121_{—}1 deposited with the ATCC under accession number 98019;
(i) a polynucleotide encoding a protein comprising the amino acid sequence of SEQ ID NO:14;
(j) a polynucleotide encoding a protein comprising a fragment of the amino acid sequence of SEQ ID NO:14 having biological activity, the fragment comprising eight contiguous amino acids of SEQ ID NO:14;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:13.

Preferably, such polynucleotide comprises the nucleotide sequence of SEQ ID NO:13 from nucleotide 370 to nucleotide 1338; the nucleotide sequence of SEQ ID NO:13 from nucleotide 700 to nucleotide 1338; the nucleotide sequence of SEQ ID NO:13 from nucleotide 621 to nucleotide 1474; the nucleotide sequence of the full-length protein coding sequence of clone B121_{—}1 deposited with the ATCC under accession number 98019; or the nucleotide sequence of a mature protein coding sequence of clone B121_{—}1 deposited with the ATCC under accession number 98019. In other preferred embodiments, the polynucleotide encodes the full-length or a mature protein encoded by the cDNA insert of clone B121_{—}1 deposited with the ATCC under accession number 98019. In yet other preferred embodiments, the present invention provides a polynucleotide encoding a protein comprising the amino acid sequence of SEQ ID NO:14 from amino acid 85 to amino acid 323. In further preferred embodiments, the present invention provides a polynucleotide encoding a protein comprising a fragment of the amino acid sequence of SEQ ID NO:14 having biological activity, the fragment preferably comprising eight (more preferably twenty, most preferably thirty) contiguous amino acids of SEQ ID NO:14, or a polynucleotide encoding a protein comprising a fragment of the amino acid sequence of SEQ ID NO:14 having biological activity, the fragment comprising the amino acid sequence from amino acid 156 to amino acid 165 of SEQ ID NO:14.

Other embodiments provide the gene corresponding to the cDNA sequence of SEQ ID NO:13.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:13, but excluding the poly(A) tail at the 3′ end of SEQ ID NO:13; and
      (ab) the nucleotide sequence of the cDNA insert of clone B121_{—}1 deposited with the ATCC under accession number 98019;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:13, but excluding the poly(A) tail at the 3′ end of SEQ ID NO:13; and
      (bb) the nucleotide sequence of the cDNA insert of clone B121_{—}1 deposited with the ATCC under accession number 98019;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:13, and extending contiguously from a nucleotide sequence corresponding to the 5′ end of SEQ ID NO:13 to a nucleotide sequence corresponding to the 3′ end of SEQ ID NO:13, but excluding the poly(A) tail at the 3′ end of SEQ ID NO:13. Also preferably the polynucleotide isolated according to the above process comprises a nucleotide sequence corresponding to the cDNA sequence of SEQ ID NO:13 from nucleotide 370 to nucleotide 1338, and extending contiguously from a nucleotide sequence corresponding to the 5′ end of said sequence of SEQ ID NO:13 from nucleotide 370 to nucleotide 1338, to a nucleotide sequence corresponding to the 3′ end of said sequence of SEQ ID NO:13 from nucleotide 370 to nucleotide 1338. Also preferably the polynucleotide isolated according to the above process comprises a nucleotide sequence corresponding to the cDNA sequence of SEQ ID NO:13 from nucleotide 700 to nucleotide 1338, and extending contiguously from a nucleotide sequence corresponding to the 5′ end of said sequence of SEQ ID NO:13 from nucleotide 700 to nucleotide 1338, to a nucleotide sequence corresponding to the 3′ end of said sequence of SEQ ID NO:13 from nucleotide 700 to nucleotide 1338. Also preferably the polynucleotide isolated according to the above process comprises a nucleotide sequence corresponding to the cDNA sequence of SEQ ID NO:13 from nucleotide 621 to nucleotide 1474, and extending contiguously from a nucleotide sequence corresponding to the 5′ end of said sequence of SEQ ID NO:13 from nucleotide 621 to nucleotide 1474, to a nucleotide sequence corresponding to the 3′ end of said sequence of SEQ ID NO:13 from nucleotide 621 to nucleotide 1474.

In other embodiments, the present invention provides a composition comprising a protein, wherein said protein comprises an amino acid sequence selected from the group consisting of:
(a) the amino acid sequence of SEQ ID NO:14;
(b) the amino acid sequence of SEQ ID NO:14 from amino acid 85 to amino acid 323;
(c) a fragment of the amino acid sequence of SEQ ID NO:14, the fragment comprising eight contiguous amino acids of SEQ ID NO: 14; and
(d) the amino acid sequence encoded by the cDNA insert of clone B121_{—}1 deposited with the ATCC under accession number 98019;
   the protein being substantially free from other mammalian proteins. Preferably such protein comprises the amino acid sequence of SEQ ID NO:14 or the amino acid sequence of SEQ ID NO:14 from amino acid 85 to amino acid 323. In further preferred embodiments, the present invention provides a protein comprising a fragment of the amino acid sequence of SEQ ID NO:14 having biological activity, the fragment preferably comprising eight (more preferably twenty, most preferably thirty) contiguous amino acids of SEQ ID NO:14, or a protein comprising a fragment of the amino acid sequence of SEQ ID NO:14 having biological activity, the fragment comprising the amino acid sequence from amino acid 156 to amino acid 165 of SEQ ID NO:14.

In one embodiment, the present invention provides a composition comprising an isolated polynucleotide selected from the group consisting of:
(a) a polynucleotide comprising the nucleotide sequence of SEQ ID NO:15;
(b) a polynucleotide comprising the nucleotide sequence of SEQ ID NO:15 from nucleotide 361 to nucleotide 696;
(c) a polynucleotide comprising the nucleotide sequence of SEQ ID NO:15 from nucleotide 673 to nucleotide 696;
(d) a polynucleotide comprising the nucleotide sequence of SEQ ID NO:15 from nucleotide 352 to nucleotide 520;
(e) a polynucleotide comprising the nucleotide sequence of the full-length protein coding sequence of clone B196_{—}122 deposited with the ATCC under accession number 98021;
(f) a polynucleotide encoding the full-length protein encoded by the cDNA insert of clone B196_{—}122 deposited with the ATCC under accession number 98021;
(g) a polynucleotide comprising the nucleotide sequence of a mature protein coding sequence of clone B196_{—}122 deposited with the ATCC under accession number 98021;
(h) a polynucleotide encoding a mature protein encoded by the cDNA insert of clone B196_{—}122 deposited with the ATCC under accession number 98021;
(i) a polynucleotide encoding a protein comprising the amino add sequence of SEQ ID NO:16;
(j) a polynucleotide encoding a protein comprising a fragment of the amino acid sequence of SEQ ID NO:16 having biological activity, the fragment comprising eight contiguous amino acids of SEQ ID NO:16;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:15.

Preferably, such polynucleotide comprises the nucleotide sequence of SEQ ID NO:15 from nucleotide 361 to nucleotide 696; the nucleotide sequence of SEQ ID NO:15 from nucleotide 673 to nucleotide 696; the nucleotide sequence of SEQ ID NO:15 from nucleotide 352 to nucleotide 520; the nucleotide sequence of the full-length protein coding sequence of clone B196_{—}122 deposited with the ATCC under accession number 98021; or the nucleotide sequence of a mature protein coding sequence of clone B196_{—}122 deposited with the ATCC under accession number 98021. In other preferred embodiments, the polynucleotide encodes the full-length or a mature protein encoded by the cDNA insert of clone B196_{—}122 deposited with the ATCC under accession number 98021. In yet other preferred embodiments, the present invention provides a polynucleotide encoding a protein comprising the amino acid sequence of SEQ ID NO:16 from amino acid 55 to amino acid 99. In further preferred embodiments, the present invention provides a polynucleotide encoding a protein comprising a fragment of the amino acid sequence of SEQ ID NO:16 having biological activity, the fragment preferably comprising eight (more preferably twenty, most preferably thirty) contiguous amino acids of SEQ ID NO:16, or a polynucleotide encoding a protein comprising a fragment of the amino acid sequence of SEQ ID NO:16 having biological activity, the fragment comprising the amino acid sequence from amino acid 51 to amino acid 60 of SEQ ID NO:16.

Other embodiments provide the gene corresponding to the cDNA sequence of SEQ ID NO:15.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:15, but excluding the poly(A) tail at the 3′ end of SEQ ID NO:15; and
      (ab) the nucleotide sequence of the cDNA insert of clone B196_{—}122 deposited with the ATCC under accession number 98021;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:15, but excluding the poly(A) tail at the 3′ end of SEQ. ID NO:15; and
      (bb) the nucleotide sequence of the cDNA insert of clone B196_{—}122 deposited with the ATCC under accession number 98021;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:15, and extending contiguously from a nucleotide sequence corresponding to the 5′ end of SEQ ID NO:15 to a nucleotide sequence corresponding to the 3′ end of SEQ ID NO:15, but excluding the poly(A) tail at the 3′ end of SEQ ID NO:15. Also preferably the polynucleotide isolated according to the above process comprises a nucleotide sequence corresponding to the cDNA sequence of SEQ ID NO:15 from nucleotide 361 to nucleotide 696, and extending contiguously from a nucleotide sequence corresponding to the 5′ end of said sequence of SEQ ID NO:15 from nucleotide 361 to nucleotide 696, to a nucleotide sequence corresponding to the 3′ end of said sequence of SEQ ID NO:15 from nucleotide 361 to nucleotide 696. Also preferably the polynucleotide isolated according to the above process comprises a nucleotide sequence corresponding to the cDNA sequence of SEQ ID NO:15 from nucleotide 673 to nucleotide 696, and extending contiguously from a nucleotide sequence corresponding to the 5′ end of said sequence of SEQ ID NO:15 from nucleotide 673 to nucleotide 696, to a nucleotide sequence corresponding to the 3′ end of said sequence of SEQ ID NO:15 from nucleotide 673 to nucleotide 696. Also preferably the polynucleotide isolated according to the above process comprises a nucleotide sequence corresponding to the cDNA sequence of SEQ ID NO:15 from nucleotide 352 to nucleotide 520, and extending contiguously from a nucleotide sequence corresponding to the 5′ end of said sequence of SEQ ID NO:15 from nucleotide 352 to nucleotide 520, to a nucleotide sequence corresponding to the 3′ end of said sequence of SEQ ID NO:15 from nucleotide 352 to nucleotide 520.

In other embodiments, the present invention provides a composition comprising a protein, wherein said protein comprises an amino acid sequence selected from the group consisting of:
(a) the amino acid sequence of SEQ ID NO:16;
(b) the amino acid sequence of SEQ ID NO:16 from amino acid 55 to amino acid 99;
(c) a fragment of the amino acid sequence of SEQ ID NO:16, the fragment comprising eight contiguous amino acids of SEQ ID NO:16; and
(d) the amino acid sequence encoded by the cDNA insert of clone B196_{—}122 deposited with the ATCC under accession number 98021;
   the protein being substantially free from other mammalian proteins. Preferably such protein comprises the amino acid sequence of SEQ ID NO:16 or the amino acid sequence of SEQ ID NO:16 from amino acid 55 to amino acid 99. In further preferred embodiments, the present invention provides a protein comprising a fragment of the amino acid sequence of SEQ ID NO:16 having biological activity, the fragment preferably comprising eight (more preferably twenty, most preferably thirty) contiguous amino acids of SEQ ID NO:16, or a protein comprising a fragment of the amino acid sequence of SEQ ID NO:16 having biological activity, the fragment comprising the amino acid sequence from amino acid 51 to amino acid 60 of SEQ ID NO:16.

In one embodiment, the present invention provides a composition comprising an isolated polynucleotide selected from the group consisting of:
(a) a polynucleotide comprising the nucleotide sequence of SEQ ID NO:17;
(b) a polynucleotide comprising the nucleotide sequence of SEQ ID NO:17 from nucleotide 33 to nucleotide 521;
(c) a polynucleotide comprising the nucleotide sequence of SEQ ID NO:17 from nucleotide 60 to nucleotide 521;
(d) a polynucleotide comprising the nucleotide sequence of the full-length protein coding sequence of clone D157_{—}4 deposited with the ATCC under accession number 98020;
(e) a polynucleotide encoding the full-length protein encoded by the cDNA insert of clone D157_{—}4 deposited with the ATCC under accession number 98020;
(f) a polynucleotide comprising the nucleotide sequence of a mature protein coding sequence of clone D157_{—}4 deposited with the ATCC under accession number 98020;
(g) a polynucleotide encoding a mature protein encoded by the cDNA insert of clone D157_{—}4 deposited with the ATCC under accession number 98020;
(h) a polynucleotide encoding a protein comprising the amino acid sequence of SEQ ID NO:18;
(i) a polynucleotide encoding a protein comprising a fragment of the amino acid sequence of SEQ ID NO:18 having biological activity, the fragment comprising eight contiguous amino acids of SEQ ID NO:18;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:17.

Preferably, such polynucleotide comprises the nucleotide sequence of SEQ ID NO:17 from nucleotide 33 to nucleotide 521; the nucleotide sequence of SEQ ID NO:17 from nucleotide 60 to nucleotide 521; the nucleotide sequence of the full-length protein coding sequence of clone D157_{—}4 deposited with the ATCC under accession number 98020; or the nucleotide sequence of a mature protein coding sequence of clone D157_{—}4 deposited with the ATCC under accession number 98020. In other preferred embodiments, the polynucleotide encodes the full-length or a mature protein encoded by the cDNA insert of clone D157_{—}4 deposited with the ATCC under accession number 98020. In further preferred embodiments, the present invention provides a polynucleotide encoding a protein comprising a fragment of the amino acid sequence of SEQ ID NO:18 having biological activity, the fragment preferably comprising eight (more preferably twenty, most preferably thirty) contiguous amino acids of SEQ ID NO:18, or a polynucleotide encoding a protein comprising a fragment of the amino acid sequence of SEQ ID NO:18 having biological activity, the fragment comprising the amino acid sequence from amino acid 76 to amino acid 85 of SEQ ID NO:18.

Other embodiments provide the gene corresponding to the cDNA sequence of SEQ ID NO:17.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:17, but excluding the poly(A) tail at the 3′ end of SEQ ID NO:17; and
      (ab) the nucleotide sequence of the cDNA insert of clone D157_{—}4 deposited with the ATCC under accession number 98020;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:17, but excluding the poly(A) tail at the 3′ end of SEQ ID NO:17; and
      (bb) the nucleotide sequence of the cDNA insert of clone D157_{—}4 deposited with the ATCC under accession number 98020;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:17, and extending contiguously from a nucleotide sequence corresponding to the 5′ end of SEQ ID NO:17 to a nucleotide sequence corresponding to the 3′ end of SEQ ID NO:17, but excluding the poly(A) tail at the 3′ end of SEQ ID NO:17. Also preferably the polynucleotide isolated according to the above process comprises a nucleotide sequence corresponding to the cDNA sequence of SEQ ID NO:17 from nucleotide 33 to nucleotide 521, and extending contiguously from a nucleotide sequence corresponding to the 5′ end of said sequence of SEQ ID NO:17 from nucleotide 33 to nucleotide 521, to a nucleotide sequence corresponding to the 3′ end of said sequence of SEQ ID NO:17 from nucleotide 33 to nucleotide 521. Also preferably the polynucleotide isolated according to the above process comprises a nucleotide sequence corresponding to the cDNA sequence of SEQ ID NO:17 from nucleotide 60 to nucleotide 521, and extending contiguously from a nucleotide sequence corresponding to the 5′ end of said sequence of SEQ ID NO:17 from nucleotide 60 to nucleotide 521, to a nucleotide sequence corresponding to the 3′ end of said sequence of SEQ ID NO:17 from nucleotide 60 to nucleotide 521.

In other embodiments, the present invention provides a composition comprising a protein, wherein said protein comprises an amino acid sequence selected from the group consisting of:
(a) the amino acid sequence of SEQ ID NO:18;
(b) a fragment of the amino acid sequence of SEQ ID NO:18, the fragment comprising eight contiguous amino acids of SEQ ID NO:18; and
(c) the amino acid sequence encoded by the cDNA insert of clone D157_{—}4 deposited with the ATCC under accession number 98020;
   the protein being substantially free from other mammalian proteins. Preferably such protein comprises the amino acid sequence of SEQ ID NO:18. In further preferred embodiments, the present invention provides a protein comprising a fragment of the amino acid sequence of SEQ ID NO:18 having biological activity, the fragment preferably comprising eight (more preferably twenty, most preferably thirty) contiguous amino acids of SEQ ID NO:18, or a protein comprising a fragment of the amino acid sequence of SEQ ID NO:18 having biological activity, the fragment comprising the amino acid sequence from amino acid 76 to amino acid 85 of SEQ ID NO:18.

In one embodiment, the present invention provides a composition comprising an isolated polynucleotide selected from the group consisting of:
(a) a polynucleotide comprising the nucleotide sequence of SEQ ID NO:19;
(b) a polynucleotide comprising the nucleotide sequence of SEQ ID NO:19 from nucleotide 21 to nucleotide 608;
(c) a polynucleotide comprising the nucleotide sequence of SEQ ID NO:19 from nucleotide 289 to nucleotide 475;
(d) a polynucleotide comprising the nucleotide sequence of the full-length protein coding sequence of clone B219_{—}2 deposited with the ATCC under accession number 98028;
(e) a polynucleotide encoding the full-length protein encoded by the cDNA insert of clone B219_{—}2 deposited with the ATCC under accession number 98028;
(f) a polynucleotide comprising the nucleotide sequence of a mature protein coding sequence of clone B219_{—}2 deposited with the ATCC under accession number 98028;
(g) a polynucleotide encoding a mature protein encoded by the cDNA insert of clone B219_{—}2 deposited with the ATCC under accession number 98028;
(h) a polynucleotide encoding a protein comprising the amino acid sequence of SEQ ID NO:20;
(i) a polynucleotide encoding a protein comprising a fragment of the amino acid sequence of SEQ ID NO:20 having biological activity, the fragment comprising eight contiguous amino acids of SEQ ID NO:20;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:19.

Preferably, such polynucleotide comprises the nucleotide sequence of SEQ ID NO:19 from nucleotide 21 to nucleotide 608; the nucleotide sequence of SEQ ID NO:19 from nucleotide 289 to nucleotide 475; the nucleotide sequence of the full-length protein coding sequence of clone B219_{—}2 deposited with the ATCC under accession number 98028; or the nucleotide sequence of a mature protein coding sequence of clone B219_{—}2 deposited with the ATCC under accession number 98028. In other preferred embodiments, the polynucleotide encodes the full-length or a mature protein encoded by the cDNA insert of clone B219_{—}2 deposited with the ATCC under accession number 98028. In yet other preferred embodiments, the present invention provides a polynucleotide encoding a protein comprising the amino acid sequence of SEQ ID NO:20 from amino acid 106 to amino acid 143. In further preferred embodiments, the present invention provides a polynucleotide encoding a protein comprising a fragment of the amino acid sequence of SEQ ID NO:20 having biological activity, the fragment preferably comprising eight (more preferably twenty, most preferably thirty) contiguous amino acids of SEQ ID NO:20, or a polynucleotide encoding a protein comprising a fragment of the amino acid sequence of SEQ ID NO:20 having biological activity, the fragment comprising the amino acid sequence from amino acid 93 to amino acid 102 of SEQ ID NO:20.

Other embodiments provide the gene corresponding to the cDNA sequence of SEQ ID NO:19.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO 19, but excluding the poly(A) tail at the 3′ end of SEQ ID NO:19; and
      (ab) the nucleotide sequence of the cDNA insert of clone B219_{—}2 deposited with the ATCC under accession number 98028;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:19, but excluding the poly(A) tail at the 3′ end of SEQ ID NO:19; and
      (bb) the nucleotide sequence of the cDNA insert of clone B219_{—}2 deposited with the ATCC under accession number 98028;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:19, and extending contiguously from a nucleotide sequence corresponding to the 5′ end of SEQ ID NO:19 to a nucleotide sequence corresponding to the 3′ end of SEQ ID NO:19, but excluding the poly(A) tail at the 3′ end of SEQ ID NO:19. Also preferably the polynucleotide isolated according to the above process comprises a nucleotide sequence corresponding to the cDNA sequence of SEQ ID NO:19 from nucleotide 21 to nucleotide 608, and extending contiguously from a nucleotide sequence corresponding to the 5′ end of said sequence of SEQ ID NO:19 from nucleotide 21 to nucleotide 608, to a nucleotide sequence corresponding to the 3′ end of said sequence of SEQ ID NO:19 from nucleotide 21 to nucleotide 608. Also preferably the polynucleotide isolated according to the above process comprises a nucleotide sequence corresponding to the cDNA sequence of SEQ ID NO:19 from nucleotide 289 to nucleotide 475, and extending contiguously from a nucleotide sequence corresponding to the 5′ end of said sequence of SEQ ID NO:19 from nucleotide 289 to nucleotide 475, to a nucleotide sequence corresponding to the 3′ end of said sequence of SEQ ID NO:19 from nucleotide 289 to nucleotide 475.

In other embodiments, the present invention provides a composition comprising a protein, wherein said protein comprises an amino acid sequence selected from the group consisting of:
(a) the amino acid sequence of SEQ ID NO:20;
(b) the amino acid sequence of SEQ ID NO:20 from amino acid 106 to amino acid 143;
(c) a fragment of the amino acid sequence of SEQ ID NO:20, the fragment comprising eight contiguous amino acids of SEQ ID NO:20; and
(d) the amino acid sequence encoded by the cDNA insert of clone B219_{—}2 deposited with the ATCC under accession number 98028;
   the protein being substantially free from other mammalian proteins. Preferably such protein comprises the amino acid sequence of SEQ ID NO:20 or the amino acid sequence of SEQ ID NO:20 from amino acid 106 to amino acid 143. In further preferred embodiments, the present invention provides a protein comprising a fragment of the amino acid sequence of SEQ ID NO:20 having biological activity, the fragment preferably comprising eight (more preferably twenty, most preferably thirty) contiguous amino acids of SEQ ID NO:20, or a protein comprising a fragment of the amino acid sequence of SEQ ID NO:20 having biological activity, the fragment comprising the amino acid sequence from amino acid 93 to amino acid 102 of SEQ ID NO:20.

In one embodiment, the present invention provides a composition comprising an isolated polynucleotide selected from the group consisting of:
(a) a polynucleotide comprising the nucleotide sequence of SEQ ID NO:21;
(b) a polynucleotide comprising the nucleotide sequence of SEQ ID NO:21 from nucleotide 732 to nucleotide 1274;
(c) a polynucleotide comprising the nucleotide sequence of SEQ ID NO:21 from nucleotide 852 to nucleotide 1274;
(d) a polynucleotide comprising the nucleotide sequence of SEQ ID NO:21 from nucleotide 411 to nucleotide 854;
(e) a polynucleotide comprising the nucleotide sequence of the full-length protein coding sequence of clone G52_{—}24 deposited with the ATCC under accession number 98028;
(f) a polynucleotide encoding the full-length protein encoded by the cDNA insert of clone G52_{—}24 deposited with the ATCC under accession number 98028;
(g) a polynucleotide comprising the nucleotide sequence of a mature protein coding sequence of clone G52_{—}24 deposited with the ATCC under accession number 98028;
(h) a polynucleotide encoding a mature protein encoded by the cDNA insert of clone G52_{—}24 deposited with the ATCC under accession number 98028;
(i) a polynucleotide encoding a protein comprising the amino acid sequence of SEQ ID NO:22;
(j) a polynucleotide encoding a protein comprising a fragment of the amino acid sequence of SEQ ID NO:22 having biological activity, the fragment comprising eight contiguous amino acids of SEQ ID NO:22;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:21.

Preferably, such polynucleotide comprises the nucleotide sequence of SEQ ID NO:21 from nucleotide 732 to nucleotide 1274; the nucleotide sequence of SEQ ID NO:21 from nucleotide 852 to nucleotide 1274; the nucleotide sequence of SEQ ID NO:21 from nucleotide 411 to nucleotide 854; the nucleotide sequence of the full-length protein coding sequence of clone G52_{—}24 deposited with the ATCC under accession number 98028; or the nucleotide sequence of a mature protein coding sequence of clone G52_{—}24 deposited with the ATCC under accession number 98028. In other preferred embodiments, the polynucleotide encodes the full-length or a mature protein encoded by the cDNA insert of clone G52_{—}24 deposited with the ATCC under accession number 98028. In yet other preferred embodiments, the present invention provides a polynucleotide encoding a protein comprising the amino acid sequence of SEQ ID NO:22 from amino acid 93 to amino acid 114. In further preferred embodiments, the present invention provides a polynucleotide encoding a protein comprising a fragment of the amino acid sequence of SEQ ID NO:22 having biological activity, the fragment preferably comprising eight (more preferably twenty, most preferably thirty) contiguous amino acids of SEQ ID NO:22, or a polynucleotide encoding a protein comprising a fragment of the amino acid sequence of SEQ ID NO:22 having biological activity, the fragment comprising the amino acid sequence from amino acid 85 to amino acid 94 of SEQ ID NO:22.

Other embodiments provide the gene corresponding to the cDNA sequence of SEQ ID NO:21.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:21, but excluding the poly(A) tail at the 3′ end of SEQ ID NO:21; and
      (ab) the nucleotide sequence of the cDNA insert of clone G52_{—}24 deposited with the ATCC under accession number 98028;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:21, but excluding the poly(A) tail at the 3′ end of SEQ ID NO:21; and
      (bb) the nucleotide sequence of the cDNA insert of clone G52_{—}24 deposited with the ATCC under accession number 98028;
   (ii) hybridizing said primer(s) to human genomic DNA in conditions at least as stringent as 4×SSC at 50 degrees C.;
   (ii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:21, and extending contiguously from a nucleotide sequence corresponding to the 5′ end of SEQ ID NO:21 to a nucleotide sequence corresponding to the 3′ end of SEQ ID NO:21, but excluding the poly(A) tail at the 3′ end of SEQ ID NO:21. Also preferably the polynucleotide isolated according to the above process comprises a nucleotide sequence corresponding to the cDNA sequence of SEQ ID NO:21 from nucleotide 732 to nucleotide 1274, and extending contiguously from a nucleotide sequence corresponding to the 5′ end of said sequence of SEQ ID NO:21 from nucleotide 732 to nucleotide 1274, to a nucleotide sequence corresponding to the 3′ end of said sequence of SEQ ID NO:21 from nucleotide 732 to nucleotide 1274. Also preferably the polynucleotide isolated according to the above process comprises a nucleotide sequence corresponding to the cDNA sequence of SEQ ID NO:21 from nucleotide 852 to nucleotide 1274, and extending contiguously from a nucleotide sequence corresponding to the 5′ end of said sequence of SEQ ID NO:21 from nucleotide 852 to nucleotide 1274, to a nucleotide sequence corresponding to the 3′ end of said sequence of SEQ ID NO:21 from nucleotide 852 to nucleotide 1274. Also preferably the polynucleotide isolated according to the above process comprises a nucleotide sequence corresponding to the cDNA sequence of SEQ ID NO:21 from nucleotide 411 to nucleotide 854, and extending contiguously from a nucleotide sequence corresponding to the 5′ end of said sequence of SEQ ID NO:21 from nucleotide 411 to nucleotide 854, to a nucleotide sequence corresponding to the 3′ end of said sequence of SEQ ID NO:21 from nucleotide 411 to nucleotide 854.

In other embodiments, the present invention provides a composition comprising a protein, wherein said protein comprises an amino add sequence selected from the group consisting of:
(a) the amino acid sequence of SEQ ID NO:22;
(b) the amino acid sequence of SEQ ID NO:22 from amino acid 93 to amino acid 114;
(c) a fragment of the amino acid sequence of SEQ ID NO:22, the fragment comprising eight contiguous amino acids of SEQ ID NO:22; and
(d) the amino acid sequence encoded by the cDNA insert of clone G52_{—}24 deposited with the ATCC under accession number 98028;
   the protein being substantially free from other mammalian proteins. Preferably such protein comprises the amino acid sequence of SEQ ID NO:22 or the amino acid sequence of SEQ ID NO:22 from amino acid 93 to amino acid 114. In further preferred embodiments, the present invention provides a protein comprising a fragment of the amino acid sequence of SEQ ID NO:22 having biological activity, the fragment preferably comprising eight (more preferably twenty, most preferably thirty) contiguous amino acids of SEQ ID NO:22, or a protein comprising a fragment of the amino acid sequence of SEQ ID NO:22 having biological activity, the fragment comprising the amino acid sequence from amino acid 85 to amino acid 94 of SEQ ID NO:22.

In one embodiment, the present invention provides a composition comprising an isolated polynucleotide selected from the group consisting of:
(a) a polynucleotide comprising the nucleotide sequence of SEQ ID NO:23;
(b) a polynucleotide comprising the nucleotide sequence of SEQ ID NO:23 from nucleotide 68 to nucleotide 2995;
(c) a polynucleotide comprising the nucleotide sequence of SEQ ID NO:23 from nucleotide 353 to nucleotide 2995;
(d) a polynucleotide comprising the nucleotide sequence of SEQ ID NO:23 from nucleotide 1 to nucleotide 2712;
(e) a polynucleotide comprising the nucleotide sequence of the full-length protein coding sequence of clone G86_{—}2 deposited with the ATCC under accession number 98028;
(f) a polynucleotide encoding the full-length protein encoded by the cDNA insert of clone G86_{—}2 deposited with the ATCC under accession number 98028;
(g) a polynucleotide comprising the nucleotide sequence of a mature protein coding sequence of clone G86_{—}2 deposited with the ATCC under accession number 98028;
(h) a polynucleotide encoding a mature protein encoded by the cDNA insert of clone G86_{—}2 deposited with the ATCC under accession number 98028;
(i) a polynucleotide encoding a protein comprising the amino acid sequence of SEQ ID NO:24;
(j) a polynucleotide encoding a protein comprising a fragment of the amino acid sequence of SEQ ID NO:24 having biological activity, the fragment comprising eight contiguous amino acids of SEQ ID NO:24;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:23.

Preferably, such polynucleotide comprises the nucleotide sequence of SEQ ID NO:23 from nucleotide 68 to nucleotide 2995; the nucleotide sequence of SEQ ID NO:23 from nucleotide 353 to nucleotide 2995; the nucleotide sequence of SEQ ID NO:23 from nucleotide 1 to nucleotide 2712; the nucleotide sequence of the full-length protein coding sequence of clone G86_{—}2 deposited with the ATCC under accession number 98028; or the nucleotide sequence of a mature protein coding sequence of clone G86_{—}2 deposited with the ATCC under accession number 98028. In other preferred embodiments, the polynucleotide encodes the full-length or a mature protein encoded by the cDNA insert of clone G86_{—}2 deposited with the ATCC under accession number 98028. In yet other preferred embodiments, the present invention provides a polynucleotide encoding a protein comprising the amino acid sequence of SEQ ID NO:24 from amino acid 1 to amino acid 96. In further preferred embodiments, the present invention provides a polynucleotide encoding a protein comprising a fragment of the amino acid sequence of SEQ ID NO:24 having biological activity, the fragment preferably comprising eight (more preferably twenty, most preferably thirty) contiguous amino acids of SEQ ID NO:24, or a polynucleotide encoding a protein comprising a fragment of the amino acid sequence of SEQ ID NO:24 having biological activity, the fragment comprising the amino acid sequence from amino acid 483 to amino acid 492 of SEQ ID NO:24.

Other embodiments provide the gene corresponding to the cDNA sequence of SEQ ID NO:23.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:23, but excluding the poly(A) tail at the 3′ end of SEQ ID NO:23; and
      (ab) the nucleotide sequence of the cDNA insert of clone G86_{—}2 deposited with the ATCC under accession number 98028;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:23, but excluding the poly(A) tail at the 3′ end of SEQ ID NO:23; and
      (bb) the nucleotide sequence of the cDNA insert of clone G86_{—}2 deposited with the ATCC under accession number 98028;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:23, and extending contiguously from a nucleotide sequence corresponding to the 5′ end of SEQ ID NO:23 to a nucleotide sequence corresponding to the 3′ end of SEQ ID NO:23, but excluding the poly(A) tail at the 3′ end of SEQ ID NO:23. Also preferably the polynucleotide isolated according to the above process comprises a nucleotide sequence corresponding to the cDNA sequence of SEQ ID NO:23 from nucleotide 68 to nucleotide 2995, and extending contiguously from a nucleotide sequence corresponding to the 5′ end of said sequence of SEQ ID NO:23 from nucleotide 68 to nucleotide 2995, to a nucleotide sequence corresponding to the 3′ end of said sequence of SEQ ID NO:23 from nucleotide 68 to nucleotide 2995. Also preferably the polynucleotide isolated according to the above process comprises a nucleotide sequence corresponding to the cDNA sequence of SEQ ID NO:23 from nucleotide 353 to nucleotide 2995, and extending contiguously from a nucleotide sequence corresponding to the 5′ end of said sequence of SEQ ID NO:23 from nucleotide 353 to nucleotide 2995, to a nucleotide sequence corresponding to the 3′ end of said sequence of SEQ ID NO:23 from nucleotide 353 to nucleotide 2995. Also preferably the polynucleotide isolated according to the above process comprises a nucleotide sequence corresponding to the cDNA sequence of SEQ ID NO:23 from nucleotide I to nucleotide 2712, and extending contiguously from a nucleotide sequence corresponding to the 5′ end of said sequence of SEQ ID NO:23 from nucleotide 1 to nucleotide 2712, to a nucleotide sequence corresponding to the 3′ end of said sequence of SEQ ID NO:23 from nucleotide 1 to nucleotide 2712.

In other embodiments, the present invention provides a composition comprising a protein, wherein said protein comprises an amino acid sequence selected from the group consisting of:
(a) the amino acid sequence of SEQ ID NO:24;
(b) the amino acid sequence of SEQ ID NO:24 from amino acid 1 to amino acid 96;
(c) a fragment of the amino acid sequence of SEQ ID NO:24, the fragment comprising eight contiguous amino acids of SEQ ID NO:24; and
(d) the amino acid sequence encoded by the cDNA insert of clone G86_{—}2 deposited with the ATCC under accession number 98028;
   the protein being substantially free from other mammalian proteins. Preferably such protein comprises the amino acid sequence of SEQ ID NO:24 or the amino acid sequence of SEQ ID NO:24 from amino acid 1 to amino acid 96. In further preferred embodiments, the present invention provides a protein comprising a fragment of the amino acid sequence of SEQ ID NO:24 having biological activity, the fragment preferably comprising eight (more preferably twenty, most preferably thirty) contiguous amino acids of SEQ ID NO:24, or a protein comprising a fragment of the amino acid sequence of SEQ ID NO:24 having biological activity, the fragment comprising the amino acid sequence from amino acid 483 to amino acid 492 of SEQ ID NO:24.

In one embodiment, the present invention provides a composition comprising an isolated polynucleotide selected from the group consisting of:
(a) a polynucleotide comprising the nucleotide sequence of SEQ ID NO:25;
(b) a polynucleotide comprising the nucleotide sequence of SEQ ID NO:25 from nucleotide 56 to nucleotide 847;
(c) a polynucleotide comprising the nucleotide sequence of SEQ ID NO:25 from nucleotide 105 to nucleotide 448;
(d) a polynucleotide comprising the nucleotide sequence of the full-length protein coding sequence of clone H83_{—}22 deposited with the ATCC under accession number 98028;
(e) a polynucleotide encoding the full-length protein encoded by the cDNA insert of clone H83_{—}22 deposited with the ATCC under accession number 98028;
(f) a polynucleotide comprising the nucleotide sequence of a mature protein coding sequence of clone H83_{—}22 deposited with the ATCC under accession number 98028;
(g) a polynucleotide encoding a mature protein encoded by the cDNA insert of clone H83_{—}22 deposited with the ATCC under accession number 98028;
(h) a polynucleotide encoding a protein comprising the amino acid sequence of SEQ ID NO:26;
(i) a polynucleotide encoding a protein comprising a fragment of the amino acid sequence of SEQ ID NO:26 having biological activity, the fragment comprising eight contiguous amino acids of SEQ ID NO:26;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:25.

Preferably, such polynucleotide comprises the nucleotide sequence of SEQ ID NO:25 from nucleotide 56 to nucleotide 847; the nucleotide sequence of SEQ ID NO:25 from nucleotide 105 to nucleotide 448; the nucleotide sequence of the full-length protein coding sequence of clone H83_{—}22 deposited with the ATCC under accession number 98028; or the nucleotide sequence of a mature protein coding sequence of clone H83_{—}22 deposited with the ATCC under accession number 98028. In other preferred embodiments, the polynucleotide encodes the full-length or a mature protein encoded by the cDNA insert of clone H83_{—}22 deposited with the ATCC under accession number 98028. In yet other preferred embodiments, the present invention provides a polynucleotide encoding a protein comprising the amino acid sequence of SEQ ID NO:26 from amino acid 41 to amino acid 121. In further preferred embodiments, the present invention provides a polynucleotide encoding a protein comprising a fragment of the amino acid sequence of SEQ ID NO:26 having biological activity, the fragment preferably comprising eight (more preferably twenty, most preferably thirty) contiguous amino acids of SEQ ID NO:26, or a polynucleotide encoding a protein comprising a fragment of the amino acid sequence of SEQ ID NO:26 having biological activity, the fragment comprising the amino acid sequence from amino acid 127 to amino acid 136 of SEQ ID NO:26.

Other embodiments provide the gene corresponding to the cDNA sequence of SEQ ID NO:25.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:25, but excluding the poly(A) tail at the 3′ end of SEQ ID NO:25; and
      (ab) the nucleotide sequence of the cDNA insert of clone H83_{—}22 deposited with the ATCC under accession number 98028;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:25, but excluding the poly(A) tail at the 3′ end of SEQ ID NO:25; and
      (bb) the nucleotide sequence of the cDNA insert of clone H83_{—}22 deposited with the ATCC under accession number 98028;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:25, and extending contiguously from a nucleotide sequence corresponding to the 5′ end of SEQ ID NO:25 to a nucleotide sequence corresponding to the 3′ end of SEQ ID NO:25, but excluding the poly(A) tail at the 3′ end of SEQ ID NO:25. Also preferably the polynucleotide isolated according to the above process comprises a nucleotide sequence corresponding to the cDNA sequence of SEQ ID NO:25 from nucleotide 56 to nucleotide 847, and extending contiguously from a nucleotide sequence corresponding to the 5′ end of said sequence of SEQ ID NO:25 from nucleotide 56 to nucleotide 847, to a nucleotide sequence corresponding to the 3′ end of said sequence of SEQ ID NO:25 from nucleotide 56 to nucleotide 847. Also preferably the polynucleotide isolated according to the above process comprises a nucleotide sequence corresponding to the cDNA sequence of SEQ ID NO:25 from nucleotide 105 to nucleotide 448, and extending contiguously from a nucleotide sequence corresponding to the 5′ end of said sequence of SEQ ID NO:25 from nucleotide 105 to nucleotide 448, to a nucleotide sequence corresponding to the 3′ end of said sequence of SEQ ID NO:25 from nucleotide 105 to nucleotide 448.

In other embodiments, the present invention provides a composition comprising a protein, wherein said protein comprises an amino acid sequence selected from the group consisting of:
(a) the amino acid sequence of SEQ ID NO:26;
(b) the amino acid sequence of SEQ ID NO:26 from amino acid 41 to amino acid 121;
(c) a fragment of the amino acid sequence of SEQ ID NO:26, the fragment comprising eight contiguous amino acids of SEQ ID NO:26; and
(d) the amino acid sequence encoded by the cDNA insert of clone H83_{—}22 deposited with the ATCC under accession number 98028;
   the protein being substantially free from other mammalian proteins. Preferably such protein comprises the ammo acid sequence of SEQ ID NO:26 or the amino acid sequence of SEQ ID NO:26 from amino acid 41 to amino acid 121. In further preferred embodiments, the present invention provides a protein comprising a fragment of the amino acid sequence of SEQ ID NO:26 having biological activity, the fragment preferably comprising eight (more preferably twenty, most preferably thirty) contiguous amino acids of SEQ ID NO:26, or a protein comprising a fragment of the amino acid sequence of SEQ ID NO:26 having biological activity, the fragment comprising the amino acid sequence from amino acid 127 to amino acid 136 of SEQ ID NO:26.

In one embodiment, the present invention provides a composition comprising an isolated polynucleotide selected from the group consisting of:
(a) a polynucleotide comprising the nucleotide sequence of SEQ ID NO:27;
(b) a polynucleotide comprising the nucleotide sequence of SEQ ID NO:27 from nucleotide 7 to nucleotide 588;
(c) a polynucleotide comprising the nucleotide sequence of SEQ ID NO:27 from nucleotide 157 to nucleotide 588;
(d) a polynucleotide comprising the nucleotide sequence of SEQ ID NO:27 from nucleotide 1 to nucleotide 389;
(e) a polynucleotide comprising the nucleotide sequence of the full-length protein coding sequence of clone H298_{—}23 deposited with the ATCC under accession number 98028;
(f) a polynucleotide encoding the full-length protein encoded by the cDNA insert of clone H298_{—}23 deposited with the ATCC under accession number 98028;
(g) a polynucleotide comprising the nucleotide sequence of a mature protein coding sequence of clone H298_{—}23 deposited with the ATCC under accession number 98028;
(h) a polynucleotide encoding a mature protein encoded by the cDNA insert of clone H298_{—}23 deposited with the ATCC under accession number 98028;
(i) a polynucleotide encoding a protein comprising the amino acid sequence of SEQ ID NO:28;
(j) a polynucleotide encoding a protein comprising a fragment of the amino acid sequence of SEQ ID NO:28 having biological activity, the fragment comprising eight contiguous amino acids of SEQ ID NO:28;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:27.

Preferably, such polynucleotide comprises the nucleotide sequence of SEQ ID NO:27 from nucleotide 7 to nucleotide 588; the nucleotide sequence of SEQ ID NO:27 from nucleotide 157 to nucleotide 588; the nucleotide sequence of SEQ ID NO:27 from nucleotide 1 to nucleotide 389; the nucleotide sequence of the full-length protein coding sequence of clone H298_{—}23 deposited with the ATCC under accession number 98028; or the nucleotide sequence of a mature protein coding sequence of clone H298_{—}23 deposited with the ATCC under accession number 98028. In other preferred embodiments, the polynucleotide encodes the full-length or a mature protein encoded by the cDNA insert of clone H298_{—}23 deposited with the ATCC under accession number 98028. In yet other preferred embodiments, the present invention provides a polynucleotide encoding a protein comprising the amino acid sequence of SEQ ID NO:28 from amino acid 1 to amino acid 128. In further preferred embodiments, the present invention provides a polynucleotide encoding a protein comprising a fragment of the amino acid sequence of SEQ ID NO:28 having biological activity, the fragment preferably comprising eight (more preferably twenty, most preferably thirty) contiguous amino acids of SEQ ID NO:28, or a polynucleotide encoding a protein comprising a fragment of the amino acid sequence of SEQ ID NO:28 having biological activity, the fragment comprising the amino acid sequence from amino acid 92 to amino acid 101 of SEQ ID NO:28.

Other embodiments provide the gene corresponding to the cDNA sequence of SEQ ID NO:27.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:27, but excluding the poly(A) tail at the 3′ end of SEQ ID NO:27; and
      (ab) the nucleotide sequence of the cDNA insert of clone H298_{—}23 deposited with the ATCC under accession number 98028;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:27, but excluding the poly(A) tail at the 3′ end of SEQ ID NO:27; and
      (bb) the nucleotide sequence of the cDNA insert of clone H298_{—}23 deposited with the ATCC under accession number 98028;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:27, and extending contiguously from a nucleotide sequence corresponding to the 5′ end of SEQ ID NO:27 to a nucleotide sequence corresponding to the 3′ end of SEQ ID NO:27, but excluding the poly(A) tail at the 3′ end of SEQ ID NO:27. Also preferably the polynucleotide isolated according to the above process comprises a nucleotide sequence corresponding to the cDNA sequence of SEQ ID NO:27 from nucleotide 7 to nucleotide 588, and extending contiguously from a nucleotide sequence corresponding to the 5′ end of said sequence of SEQ ID NO:27 from nucleotide 7 to nucleotide 588, to a nucleotide sequence corresponding to the 3′ end of said sequence of SEQ ID NO:27 from nucleotide 7 to nucleotide 588. Also preferably the polynucleotide isolated according to the above process comprises a nucleotide sequence corresponding to the cDNA sequence of SEQ ID NO:27 from nucleotide 157 to nucleotide 588, and extending contiguously from a nucleotide sequence corresponding to the 5′ end of said sequence of SEQ ID NO:27 from nucleotide 157 to nucleotide 588, to a nucleotide sequence corresponding to the 3′ end of said sequence of SEQ ID NO:27 from nucleotide 157 to nucleotide 588. Also preferably the polynucleotide isolated according to the above process comprises a nucleotide sequence corresponding to the cDNA sequence of SEQ ID NO:27 from nucleotide 1 to nucleotide 389, and extending contiguously from a nucleotide sequence corresponding to the 5′ end of said sequence of SEQ ID NO:27 from nucleotide 1 to nucleotide 389, to a nucleotide sequence corresponding to the 3′ end of said sequence of SEQ ID NO:27 from nucleotide 1 to nucleotide 389.

In other embodiments, the present invention provides a composition comprising a protein, wherein said protein comprises an amino acid sequence selected from the group consisting of:
(a) the amino acid sequence of SEQ ID NO:28;
(b) the amino acid sequence of SEQ ID NO:28 from amino acid 1 to amino acid 128;
(c) a fragment of the amino acid sequence of SEQ ID NO:28, the fragment comprising eight contiguous amino acids of SEQ ID NO:28; and
(d) the amino acid sequence encoded by the cDNA insert of clone H298_{—}23 deposited with the ATCC under accession number 98028;
   the protein being substantially free from other mammalian proteins. Preferably such protein comprises the amino acid sequence of SEQ ID NO:28 or the amino acid sequence of SEQ ID NO:28 from amino acid 1 to amino acid 128. In further preferred embodiments, the present invention provides a protein comprising a fragment of the amino acid sequence of SEQ ID NO:28 having biological activity, the fragment preferably comprising eight (more preferably twenty, most preferably thirty) contiguous amino acids of SEQ ID NO:28, or a protein comprising a fragment of the amino acid sequence of SEQ ID NO:28 having biological activity, the fragment comprising the amino acid sequence from amino acid 92 to amino acid 101 of SEQ ID NO:28.

In one embodiment, the present invention provides a composition comprising an isolated polynucleotide selected from the group consisting of:
(a) a polynucleotide comprising the nucleotide sequence of SEQ ID NO:29;
(b) a polynucleotide comprising the nucleotide sequence of SEQ ID NO:29 from nucleotide 80 to nucleotide 655;
(c) a polynucleotide comprising the nucleotide sequence of SEQ ID NO:29 from nucleotide 137 to nucleotide 655;
(d) a polynucleotide comprising the nucleotide sequence of SEQ ID NO:29 from nucleotide 59 to nucleotide 402;
(e) a polynucleotide comprising the nucleotide sequence of the full-length protein coding sequence of clone H849_{—}24 deposited with the ATCC under accession number 98028;
(f) a polynucleotide encoding the full-length protein encoded by the cDNA insert of clone H849_{—}24 deposited with the ATCC under accession number 98028;
(g) a polynucleotide comprising the nucleotide sequence of a mature protein coding sequence of clone H849_{—}24 deposited with the ATCC under accession number 98028;
(h) a polynucleotide encoding a mature protein encoded by the cDNA insert of clone H849_{—}24 deposited with the ATCC under accession number 98028;
(i) a polynucleotide encoding a protein comprising the amino acid sequence of SEQ ID NO:30;
(j) a polynucleotide encoding a protein comprising a fragment of the amino acid sequence of SEQ ID NO:30 having biological activity, the fragment comprising eight contiguous amino acids of SEQ ID NO:30;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:29.

Preferably, such polynucleotide comprises the nucleotide sequence of SEQ ID NO:29 from nucleotide 80 to nucleotide 655; the nucleotide sequence of SEQ ID NO:29 from nucleotide 137 to nucleotide 655; the nucleotide sequence of SEQ ID NO:29 from nucleotide 59 to nucleotide 402; the nucleotide sequence of the full-length protein coding sequence of clone H849_{—}24 deposited with the ATCC under accession number 98028; or the nucleotide sequence of a mature protein coding sequence of clone H849_{—}24 deposited with the ATCC under accession number 98028. In other preferred embodiments, the polynucleotide encodes the full-length or a mature protein encoded by the cDNA insert of clone H849_{—}24 deposited with the ATCC under accession number 98028. In yet other preferred embodiments, the present invention provides a polynucleotide encoding a protein comprising the amino acid sequence of SEQ ID NO:30 from amino acid 1 to amino acid 107. In further preferred embodiments, the present invention provides a polynucleotide encoding a protein comprising a fragment of the amino acid sequence of SEQ ID NO:30 having biological activity, the fragment preferably comprising eight (more preferably twenty, most preferably thirty) contiguous amino acids of SEQ ID NO:30, or a polynucleotide encoding a protein comprising a fragment of the amino acid sequence of SEQ ID NO:30 having biological activity, the fragment comprising the amino acid sequence from amino acid 91 to amino acid 100 of SEQ ID NO:30.

Other embodiments provide the gene corresponding to the cDNA sequence of SEQ ID NO:29.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:29, but excluding the poly(A) tail at the 3′ end of SEQ ID NO:29; and
      (ab) the nucleotide sequence of the cDNA insert of clone H849_{—}24 deposited with the ATCC under accession number 98028;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:29, but excluding the poly(A) tail at the 3′ end of SEQ ID NO:29; and
      (bb) the nucleotide sequence of the cDNA insert of clone H849_{—}24 deposited with the ATCC under accession number 98028;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:29, and extending contiguously from a nucleotide sequence corresponding to the 5′ end of SEQ ID NO:29 to a nucleotide sequence corresponding to the 3′ end of SEQ ID NO:29, but excluding the poly(A) tail at the 3′ end of SEQ ID NO:29. Also preferably the polynucleotide isolated according to the above process comprises a nucleotide sequence corresponding to the cDNA sequence of SEQ ID NO:29 from nucleotide 80 to nucleotide 655, and extending contiguously from a nucleotide sequence corresponding to the 5′ end of said sequence of SEQ ID NO:29 from nucleotide 80 to nucleotide 655, to a nucleotide sequence corresponding to the 3′ end of said sequence of SEQ ID NO:29 from nucleotide 80 to nucleotide 655. Also preferably the polynucleotide isolated according to the above process comprises a nucleotide sequence corresponding to the cDNA sequence of SEQ ID NO:29 from nucleotide 137 to nucleotide 655, and extending contiguously from a nucleotide sequence corresponding to the 5′ end of said sequence of SEQ ID NO:29 from nucleotide 137 to nucleotide 655, to a nucleotide sequence corresponding to the 3′ end of said sequence of SEQ ID NO:29 from nucleotide 137 to nucleotide 655. Also preferably the polynucleotide isolated according to the above process comprises a nucleotide sequence corresponding to the cDNA sequence of SEQ ID NO:29 from nucleotide 59 to nucleotide 402, and extending contiguously from a nucleotide sequence corresponding to the 5′ end of said sequence of SEQ ID NO:29 from nucleotide 59 to nucleotide 402, to a nucleotide sequence corresponding to the 3′ end of said sequence of SEQ ID NO:29 from nucleotide 59 to nucleotide 402.

In other embodiments, the present invention provides a composition comprising a protein, wherein said protein comprises an amino acid sequence selected from the group consisting of:
(a) the amino acid sequence of SEQ ID NO:30;
(b) the amino acid sequence of SEQ ID NO:30 from amino acid 1 to amino acid 107;
(c) a fragment of the amino acid sequence of SEQ ID NO:30, the fragment comprising eight contiguous amino acids of SEQ ID NO:30; and
(d) the amino acid sequence encoded by the cDNA insert of clone H849_{—}24 deposited with the ATCC under accession number 98028;
   the protein being substantially free from other mammalian proteins. Preferably such protein comprises the amino acid sequence of SEQ ID NO:30 or the amino acid sequence of SEQ ID NO:30 from amino acid 1 to amino acid 107. In further preferred embodiments, the present invention provides a protein comprising a fragment of the amino acid sequence of SEQ ID NO:30 having biological activity, the fragment preferably comprising eight (more preferably twenty, most preferably thirty) contiguous amino acids of SEQ ID NO:30, or a protein comprising a fragment of the amino acid sequence of SEQ ID NO:30 having biological activity, the fragment comprising the amino acid sequence from amino acid 91 to amino acid 100 of SEQ ID NO:30.

In one embodiment, the present invention provides a composition comprising an isolated polynucleotide selected from the group consisting of:
(a) a polynucleotide comprising the nucleotide sequence of SEQ ID NO:31;
(b) a polynucleotide comprising the nucleotide sequence of SEQ ID NO:31 from nucleotide 144 to nucleotide 590;
(c) a polynucleotide comprising the nucleotide sequence of SEQ ID NO:31 from nucleotide 1 to nucleotide 434;
(d) a polynucleotide comprising the nucleotide sequence of the full-length protein coding sequence of clone H905_{—}107 deposited with the ATCC under accession number 98028;
(e) a polynucleotide encoding the full-length protein encoded by the cDNA insert of clone H905_{—}107 deposited with the ATCC under accession number 98028;
(f) a polynucleotide comprising the nucleotide sequence of a mature protein coding sequence of clone H905_{—}107 deposited with the ATCC under accession number 98028;
(g) a polynucleotide encoding a mature protein encoded by the cDNA insert of clone H905_{—}107 deposited with the ATCC under accession number 98028;
(h) a polynucleotide encoding a protein comprising the amino acid sequence of SEQ ID NO:32;
(i) a polynucleotide encoding a protein comprising a fragment of the amino acid sequence of SEQ ID NO:32 having biological activity, the fragment comprising eight contiguous amino acids of SEQ ID NO:32;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:31.

Preferably, such polynucleotide comprises the nucleotide sequence of SEQ ID NO:31 from nucleotide 144 to nucleotide 590; the nucleotide sequence of SEQ ID NO:31 from nucleotide 1 to nucleotide 434; the nucleotide sequence of the full-length protein coding sequence of clone H905_{—}107 deposited with the ATCC under accession number 98028; or the nucleotide sequence of a mature protein coding sequence of clone H905_{—}107 deposited with the ATCC under accession number 98028. In other preferred embodiments, the polynucleotide encodes the full-length or a mature protein encoded by the cDNA insert of clone H905_{—}107 deposited with the ATCC under accession number 98028. In yet other preferred embodiments, the present invention provides a polynucleotide encoding a protein comprising the amino acid sequence of SEQ ID NO:32 from amino acid 1 to amino acid 97. In further preferred embodiments, the present invention provides a polynucleotide encoding a protein comprising a fragment of the amino acid sequence of SEQ ID NO:32 having biological activity, the fragment preferably comprising eight (more preferably twenty, most preferably thirty) contiguous amino acids of SEQ ID NO:32, or a polynucleotide encoding a protein comprising a fragment of the amino acid sequence of SEQ ID NO:32 having biological activity, the fragment comprising the amino acid sequence from amino acid 69 to amino acid 78 of SEQ ID NO:32.

Other embodiments provide the gene corresponding to the cDNA sequence of SEQ ID NO:31.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:31, but excluding the poly(A) tail at the 3′ end of SEQ ID NO:31; and
      (ab) the nucleotide sequence of the cDNA insert of clone H905_{—}107 deposited with the ATCC under accession number 98028;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:31, but excluding the poly(A) tail at the 3′ end of SEQ ID NO:31; and
      (bb) the nucleotide sequence of the cDNA insert of clone H905_{—}107 deposited with the ATCC under accession number 98028;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:31, and extending contiguously from a nucleotide sequence corresponding to the 5′ end of SEQ ID NO:31 to a nucleotide sequence corresponding to the 3′ end of SEQ ID NO:31, but excluding the poly(A) tail at the 3′ end of SEQ ID NO:31. Also preferably the polynucleotide isolated according to the above process comprises a nucleotide sequence corresponding to the cDNA sequence of SEQ ID NO:31 from nucleotide 144 to nucleotide 590, and extending contiguously from a nucleotide sequence corresponding to the 5′ end of said sequence of SEQ ID NO:31 from nucleotide 144 to nucleotide 590, to a nucleotide sequence corresponding to the 3′ end of said sequence of SEQ ID NO:31 from nucleotide 144 to nucleotide 590. Also preferably the polynucleotide isolated according to the above process comprises a nucleotide sequence corresponding to the cDNA sequence of SEQ ID NO:31 from nucleotide 1 to nucleotide 434, and extending contiguously from a nucleotide sequence corresponding to the 5′ end of said sequence of SEQ ID NO:31 from nucleotide 1 to nucleotide 434, to a nucleotide sequence corresponding to the 3′ end of said sequence of SEQ ID NO:31 from nucleotide 1 to nucleotide 434.

In other embodiments, the present invention provides a composition comprising a protein, wherein said protein comprises an amino acid sequence selected from the group consisting of:
(a) the amino acid sequence of SEQ ID NO:32;
(b) the amino acid sequence of SEQ ID NO:32 from amino acid 1 to amino acid 97;
(c) a fragment of the amino acid sequence of SEQ ID NO:32, the fragment comprising eight contiguous amino acids of SEQ ID NO:32; and
(d) the amino acid sequence encoded by the cDNA insert of clone H905_{—}107 deposited with the ATCC under accession number 98028;
   the protein being substantially free from other mammalian proteins. Preferably such protein comprises the amino acid sequence of SEQ ID NO:32 or the amino acid sequence of SEQ ID NO:32 from amino acid 1 to amino acid 97. In further preferred embodiments, the present invention provides a protein comprising a fragment of the amino acid sequence of SEQ ID NO:32 having biological activity, the fragment preferably comprising eight (more preferably twenty, most preferably thirty) contiguous amino acids of SEQ ID NO:32, or a protein comprising a fragment of the amino acid sequence of SEQ ID NO:32 having biological activity, the fragment comprising the amino acid sequence from amino acid 69 to amino acid 78 of SEQ ID NO:32.

In one embodiment, the present invention provides a composition comprising an isolated polynucleotide selected from the group consisting of:
(a) a polynucleotide comprising the nucleotide sequence of SEQ ID NO:33;
(b) a polynucleotide comprising the nucleotide sequence of SEQ ID NO:33 from nucleotide 19 to nucleotide 471;
(c) a polynucleotide comprising the nucleotide sequence of SEQ ID NO:33 from nucleotide 76 to nucleotide 471;
(d) a polynucleotide comprising the nucleotide sequence of SEQ ID NO:33 from nucleotide 6 to nucleotide 427;
(e) a polynucleotide comprising the nucleotide sequence of the full-length protein coding sequence of clone H1075_{—}1 deposited with the ATCC under accession number 98028;
(f) a polynucleotide encoding the full-length protein encoded by the cDNA insert of clone H1075_{—}1 deposited with the ATCC under accession number 98028;
(g) a polynucleotide comprising the nucleotide sequence of a mature protein coding sequence of clone H1075_{—}1 deposited with the ATCC under accession number 98028;
(h) a polynucleotide encoding a mature protein encoded by the cDNA insert of clone H1075_{—}1 deposited with the ATCC under accession number 98028;
(i) a polynucleotide encoding a protein comprising the amino acid sequence of SEQ ID NO:34;
(j) a polynucleotide encoding a protein comprising a fragment of the amino acid sequence of SEQ ID NO:34 having biological activity, the fragment comprising eight contiguous amino acids of SEQ ID NO:34;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:33.

Preferably, such polynucleotide comprises the nucleotide sequence of SEQ ID NO:33 from nucleotide 19 to nucleotide 471; the nucleotide sequence of SEQ ID NO:33 from nucleotide 76 to nucleotide 471; the nucleotide sequence of SEQ ID NO:33 from nucleotide 6 to nucleotide 427; the nucleotide sequence of the full-length protein coding sequence of clone H1075_{—}1 deposited with the ATCC under accession number 98028; or the nucleotide sequence of a mature protein coding sequence of clone H1075_{—}1 deposited with the ATCC under accession number 98028. In other preferred embodiments, the polynucleotide encodes the full-length or a mature protein encoded by the cDNA insert of clone H1075_{—}1 deposited with the ATCC under accession number 98028. In yet other preferred embodiments, the present invention provides a polynucleotide encoding a protein comprising the amino acid sequence of SEQ ID NO:34 from amino acid 1 to amino acid 136. In further preferred embodiments, the present invention provides a polynucleotide encoding a protein comprising a fragment of the amino acid sequence of SEQ ID NO:34 having biological activity, the fragment preferably comprising eight (more preferably twenty, most preferably thirty) contiguous amino acids of SEQ ID NO:34, or a polynucleotide encoding a protein comprising a fragment of the amino acid sequence of SEQ ID NO:34 having biological activity, the fragment comprising the amino acid sequence from amino acid 70 to amino acid 79 of SEQ ID NO:34.

Other embodiments provide the gene corresponding to the cDNA sequence of SEQ ID NO:33.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:33, but excluding the poly(A) tail at the 3′ end of SEQ ID NO:33; and
      (ab) the nucleotide sequence of the cDNA insert of clone H1075_{—}1 deposited with the ATCC under accession number 98028;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:33, but excluding the poly(A) tail at the 3′ end of SEQ ID NO:33; and
      (bb) the nucleotide sequence of the cDNA insert of clone H1075_{—}1 deposited with the ATCC under accession number 98028;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:33, and extending contiguously from a nucleotide sequence corresponding to the 5′ end of SEQ ID NO:33 to a nucleotide sequence corresponding to the 3′ end of SEQ ID NO:33, but excluding the poly(A) tail at the 3′ end of SEQ ID NO:33. Also preferably the polynucleotide isolated according to the above process comprises a nucleotide sequence corresponding to the cDNA sequence of SEQ ID NO:33 from nucleotide 19 to nucleotide 471, and extending contiguously from a nucleotide sequence corresponding to the 5′ end of said sequence of SEQ ID NO:33 from nucleotide 19 to nucleotide 471, to a nucleotide sequence corresponding to the 3′ end of said sequence of SEQ ID NO:33 from nucleotide 19 to nucleotide 471. Also preferably the polynucleotide isolated according to the above process comprises a nucleotide sequence corresponding to the cDNA sequence of SEQ ID NO:33 from nucleotide 76 to nucleotide 471, and extending contiguously from a nucleotide sequence corresponding to the 5′ end of said sequence of SEQ ID NO:33 from nucleotide 76 to nucleotide 471, to a nucleotide sequence corresponding to the 3′ end of said sequence of SEQ ID NO:33 from nucleotide 76 to nucleotide 471. Also preferably the polynucleotide isolated according to the above process comprises a nucleotide sequence corresponding to the cDNA sequence of SEQ ID NO:33 from nucleotide 6 to nucleotide 427, and extending contiguously from a nucleotide sequence corresponding to the 5′ end of said sequence of SEQ ID NO:33 from nucleotide 6 to nucleotide 427, to a nucleotide sequence corresponding to the 3′ end of said sequence of SEQ ID NO:33 from nucleotide 6 to nucleotide 427.

In other embodiments, the present invention provides a composition comprising a protein, wherein said protein comprises an amino acid sequence selected from the group consisting of:
(a) the amino acid sequence of SEQ ID NO:34;
(b) the amino acid sequence of SEQ ID NO:34 from amino acid 1 to amino acid 136;
(c) a fragment of the amino acid sequence of SEQ ID NO:34, the fragment comprising eight contiguous amino acids of SEQ ID NO:34; and
(d) the amino acid sequence encoded by the cDNA insert of clone H1075_{—}1 deposited with the ATCC under accession number 98028;
   the protein being substantially free from other mammalian proteins. Preferably such protein comprises the amino acid sequence of SEQ ID NO:34 or the amino acid sequence of SEQ ID NO:34 from amino acid 1 to amino acid 136. In further preferred embodiments, the present invention provides a protein comprising a fragment of the amino acid sequence of SEQ ID NO:34 having biological activity, the fragment preferably comprising eight (more preferably twenty, most preferably thirty) contiguous amino acids of SEQ ID NO:34, or a protein comprising a fragment of the amino acid sequence of SEQ ID NO:34 having biological activity, the fragment comprising the amino acid sequence from amino acid 70 to amino acid 79 of SEQ ID NO:34.

In one embodiment, the present invention provides a composition comprising an isolated polynucleotide selected from the group consisting of:
(a) a polynucleotide comprising the nucleotide sequence of SEQ ID NO:35;
(b) a polynucleotide comprising the nucleotide sequence of SEQ ID NO:35 from nucleotide 62 to nucleotide 703;
(c) a polynucleotide comprising the nucleotide sequence of SEQ ID NO:35 from nucleotide 614 to nucleotide 703;
(d) a polynucleotide comprising the nucleotide sequence of SEQ ID NO:35 from nucleotide 110 to nucleotide 398;
(e) a polynucleotide comprising the nucleotide sequence of the full-length protein coding sequence of clone J59_{—}41 deposited with the ATCC under accession number 98028;
(f) a polynucleotide encoding the full-length protein encoded by the cDNA insert of clone J59_{—}41 deposited with the ATCC under accession number 98028;
(g) a polynucleotide comprising the nucleotide sequence of a mature protein coding sequence of clone J59_{—}41 deposited with the ATCC under accession number 98028;
(h) a polynucleotide encoding a mature protein encoded by the cDNA insert of clone J59_{—}41 deposited with the ATCC under accession number 98028;
(i) a polynucleotide encoding a protein comprising the amino acid sequence of SEQ ID NO:36;
(j) a polynucleotide encoding a protein comprising a fragment of the amino acid sequence of SEQ ID NO:36 having biological activity, the fragment comprising eight contiguous amino acids of SEQ ID NO:36;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:35.

Preferably, such polynucleotide comprises the nucleotide sequence of SEQ ID NO:35 from nucleotide 62 to nucleotide 703; the nucleotide sequence of SEQ ID NO:35 from nucleotide 614 to nucleotide 703; the nucleotide sequence of SEQ ID NO:35 from nucleotide 110 to nucleotide 398; the nucleotide sequence of the full-length protein coding sequence of clone J59_{—}41 deposited with the ATCC under accession number 98028; or the nucleotide sequence of a mature protein coding sequence of clone J59_{—}41 deposited with the ATCC under accession number 98028. In other preferred embodiments, the polynucleotide encodes the full-length or a mature protein encoded by the cDNA insert of clone J59_{—}41 deposited with the ATCC under accession number 98028. In yet other preferred embodiments, the present invention provides a polynucleotide encoding a protein comprising the amino acid sequence of SEQ ID NO:36 from amino acid 12 to amino acid 112. In further preferred embodiments, the present invention provides a polynucleotide encoding a protein comprising a fragment of the amino acid sequence of SEQ ID NO:36 having biological activity, the fragment preferably comprising eight (more preferably twenty, most preferably thirty) contiguous amino acids of SEQ ID NO:36, or a polynucleotide encoding a protein comprising a fragment of the amino acid sequence of SEQ ID NO:36 having biological activity, the fragment comprising the amino acid sequence from amino acid 102 to amino acid 111 of SEQ ID NO:36.

Other embodiments provide the gene corresponding to the cDNA sequence of SEQ ID NO:35.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:35, but excluding the poly(A) tail at the 3′ end of SEQ ID NO:35; and
      (ab) the nucleotide sequence of the cDNA insert of clone 3° J59_{—}41 deposited with the ATCC under accession number 98028;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:35, but excluding the poly(A) tail at the 3′ end of SEQ ID NO:35; and
      (bb) the nucleotide sequence of the cDNA insert of clone J59_{—}41 deposited with the ATCC under accession number 98028;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:35, and extending contiguously from a nucleotide sequence corresponding to the 5′ end of SEQ ID NO:35 to a nucleotide sequence corresponding to the 3′ end of SEQ ID NO:35, but excluding the poly(A) tail at the 3′ end of SEQ ID NO:35. Also preferably the polynucleotide isolated according to the above process comprises a nucleotide sequence corresponding to the cDNA sequence of SEQ ID NO:35 from nucleotide 62 to nucleotide 703, and extending contiguously from a nucleotide sequence corresponding to the 5′ end of said sequence of SEQ ID NO:35 from nucleotide 62 to nucleotide 703, to a nucleotide sequence corresponding to the 3′ end of said sequence of SEQ ID NO:35 from nucleotide 62 to nucleotide 703. Also preferably the polynucleotide isolated according to the above process comprises a nucleotide sequence corresponding to the cDNA sequence of SEQ ID NO:35 from nucleotide 614 to nucleotide 703, and extending contiguously from a nucleotide sequence corresponding to the 5′ end of said sequence of SEQ ID NO:35 from nucleotide 614 to nucleotide 703, to a nucleotide sequence corresponding to the 3′ end of said sequence of SEQ ID NO:35 from nucleotide 614 to nucleotide 703. Also preferably the polynucleotide isolated according to the above process comprises a nucleotide sequence corresponding to the cDNA sequence of SEQ ID NO:35 from nucleotide 110 to nucleotide 398, and extending contiguously from a nucleotide sequence corresponding to the 5′ end of said sequence of SEQ ID NO:35 from nucleotide 110 to nucleotide 398, to a nucleotide sequence corresponding to the 3′ end of said sequence of SEQ ID NO:35 from nucleotide 110 to nucleotide 398.

In other embodiments, the present invention provides a composition comprising a protein, wherein said protein comprises an amino acid sequence selected from the group consisting of:
(a) the amino acid sequence of SEQ ID NO:36;
(b) the amino acid sequence of SEQ ID NO:36 from amino acid 12 to amino acid 112;
(c) a fragment of the amino acid sequence of SEQ ID NO:36, the fragment comprising eight contiguous amino acids of SEQ ID NO:36; and
(d) the amino acid sequence encoded by the cDNA insert of clone J59_{—}41 deposited with the ATCC under accession number 98028;
   the protein being substantially free from other mammalian proteins. Preferably such protein comprises the amino acid sequence of SEQ ID NO:36 or the amino acid sequence of SEQ ID NO:36 from amino acid 12 to amino acid 112. In further preferred embodiments, the present invention provides a protein comprising a fragment of the amino acid sequence of SEQ ID NO:36 having biological activity, the fragment preferably comprising eight (more preferably twenty, most preferably thirty) contiguous amino adds of SEQ ID NO:36, or a protein comprising a fragment of the amino acid sequence of SEQ ID NO:36 having biological activity, the fragment comprising the amino acid sequence from amino acid 102 to amino acid 111 of SEQ ID NO:36.

In one embodiment, the present invention provides a composition comprising an isolated polynucleotide selected from the group consisting of:
(a) a polynucleotide comprising the nucleotide sequence of SEQ ID NO:37;
(b) a polynucleotide comprising the nucleotide sequence of SEQ ID NO:37 from nucleotide 2 to nucleotide 910;
(c) a polynucleotide comprising the nucleotide sequence of SEQ ID NO:37 from nucleotide 221 to nucleotide 910;
(d) a polynucleotide comprising the nucleotide sequence of SEQ ID NO:37 from nucleotide 148 to nucleotide 460;
(e) a polynucleotide comprising the nucleotide sequence of the full-length protein coding sequence of clone J143_{—}1 deposited with the ATCC under accession number 98028;
(f) a polynucleotide encoding the full-length protein encoded by the cDNA insert of clone J143_{—}1 deposited with the ATCC under accession number 98028;
(g) a polynucleotide comprising the nucleotide sequence of a mature protein coding sequence of clone J143_{—}1 deposited with the ATCC under accession number 98028;
(h) a polynucleotide encoding a mature protein encoded by the cDNA insert of clone J143_{—}1 deposited with the ATCC under accession number 98028;
(i) a polynucleotide encoding a protein comprising the amino acid sequence of SEQ ID NO:38;
(j) a polynucleotide encoding a protein comprising a fragment of the amino acid sequence of SEQ ID NO:38 having biological activity, the fragment comprising eight contiguous amino acids of SEQ ID NO:38;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:37.

Preferably, such polynucleotide comprises the nucleotide sequence of SEQ ID NO:37 from nucleotide 2 to nucleotide 910; the nucleotide sequence of SEQ ID NO:37 from nucleotide 221 to nucleotide 910; the nucleotide sequence of SEQ ID NO:37 from nucleotide 148 to nucleotide 460; the nucleotide sequence of the full-length protein coding sequence of clone J143_{—}1 deposited with the ATCC under accession number 98028; or the nucleotide sequence of a mature protein coding sequence of clone J143_{—}1 deposited with the ATCC under accession number 98028. In other preferred embodiments, the polynucleotide encodes the full-length or a mature protein encoded by the cDNA insert of clone J143_{—}1 deposited with the ATCC under accession number 98028. In yet other preferred embodiments, the present invention provides a polynucleotide encoding a protein comprising the amino acid sequence of SEQ ID NO:38 from amino acid 53 to amino acid 153. In further preferred embodiments, the present invention provides a polynucleotide encoding a protein comprising a fragment of the amino acid sequence of SEQ ID NO:38 having biological activity, the fragment preferably comprising eight (more preferably twenty, most preferably thirty) contiguous amino acids of SEQ ID NO:38, or a polynucleotide encoding a protein comprising a fragment of the amino acid sequence of SEQ ID NO:38 having biological activity, the fragment comprising the amino acid sequence from amino acid 146 to amino acid 155 of SEQ ID NO:38.

Other embodiments provide the gene corresponding to the cDNA sequence of SEQ ID NO:37.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:37, but excluding the poly(A) tail at the 3′ end of SEQ ID NO:37; and
      (ab) the nucleotide sequence of the cDNA insert of clone J143_{—}1 deposited with the ATCC under accession number 98028;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:37, but excluding the poly(A) tail at the 3′ end of SEQ ID NO:37; and
      (bb) the nucleotide sequence of the cDNA insert of clone J143_{—}1 deposited with the ATCC under accession number 98028;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:37, and extending contiguously from a nucleotide sequence corresponding to the 5′ end of SEQ ID NO:37 to a nucleotide sequence corresponding to the 3′ end of SEQ ID NO:37, but excluding the poly(A) tail at the 3′ end of SEQ ID NO:37. Also preferably the polynucleotide isolated according to the above process comprises a nucleotide sequence corresponding to the cDNA sequence of SEQ ID NO:37 from nucleotide 2 to nucleotide 910, and extending contiguously from a nucleotide sequence corresponding to the 5′ end of said sequence of SEQ ID NO:37 from nucleotide 2 to nucleotide 910, to a nucleotide sequence corresponding to the 3′ end of said sequence of SEQ ID NO:37 from nucleotide 2 to nucleotide 910. Also preferably the polynucleotide isolated according to the above process comprises a nucleotide sequence corresponding to the cDNA sequence of SEQ ID NO:37 from nucleotide 221 to nucleotide 910, and extending contiguously from a nucleotide sequence corresponding to the 5′ end of said sequence of SEQ ID NO:37 from nucleotide 221 to nucleotide 910, to a nucleotide sequence corresponding to the 3′ end of said sequence of SEQ ID NO:37 from nucleotide 221 to nucleotide 910. Also preferably the polynucleotide isolated according to the above process comprises a nucleotide sequence corresponding to the cDNA sequence of SEQ ID NO:37 from nucleotide 148 to nucleotide 460, and extending contiguously from a nucleotide sequence corresponding to the 5′ end of said sequence of SEQ ID NO:37 from nucleotide 148 to nucleotide 460, to a nucleotide sequence corresponding to the 3′ end of said sequence of SEQ ID NO:37 from nucleotide 148 to nucleotide 460.

In other embodiments, the present invention provides a composition comprising a protein, wherein said protein comprises an amino acid sequence selected from the group consisting of:
(a) the amino acid sequence of SEQ ID NO:38;
(b) the amino acid sequence of SEQ ID NO:38 from amino acid 53 to amino acid 153;
(c) a fragment of the amino acid sequence of SEQ ID NO:38, the fragment comprising eight contiguous amino acids of SEQ ID NO:38; and
(d) the amino acid sequence encoded by the cDNA insert of clone J143_{—}1 deposited with the ATCC under accession number 98028;
   the protein being substantially free from other mammalian proteins. Preferably such protein comprises the amino acid sequence of SEQ ID NO:38 or the amino acid sequence of SEQ ID NO:38 from amino acid 53 to amino acid 153. In further preferred embodiments, the present invention provides a protein comprising a fragment of the amino acid sequence of SEQ ID NO:38 having biological activity, the fragment preferably comprising eight (more preferably twenty, most preferably thirty) contiguous amino acids of SEQ ID NO:38, or a protein comprising a fragment of the amino acid sequence of SEQ ID NO:38 having biological activity, the fragment comprising the amino acid sequence from amino acid 146 to amino acid 155 of SEQ ID NO:38.

In one embodiment, the present invention provides a composition comprising an isolated polynucleotide selected from the group consisting of:
(a) a polynucleotide comprising the nucleotide sequence of SEQ ID NO:39;
(b) a polynucleotide comprising the nucleotide sequence of SEQ ID NO:39 from nucleotide 1 to nucleotide 1662;
(c) a polynucleotide comprising the nucleotide sequence of SEQ ID NO:39 from nucleotide 301 to nucleotide 1662;
(d) a polynucleotide comprising the nucleotide sequence of SEQ ID NO:39 from nucleotide 1099 to nucleotide 1279;
(e) a polynucleotide comprising the nucleotide sequence of the full-length protein coding sequence of clone J218_{—}15 deposited with the ATCC under accession number 98028;
(f) a polynucleotide encoding the full-length protein encoded by the cDNA insert of clone J218_{—}15 deposited with the ATCC under accession number 98028;
(g) a polynucleotide comprising the nucleotide sequence of a mature protein coding sequence of clone J218_{—}15 deposited with the ATCC under accession number 98028;
(h) a polynucleotide encoding a mature protein encoded by the cDNA insert of clone J218_{—}15 deposited with the ATCC under accession number 98028;
(i) a polynucleotide encoding a protein comprising the amino acid sequence of SEQ ID NO:40;
(j) a polynucleotide encoding a protein comprising a fragment of the amino acid sequence of SEQ ID NO:40 having biological activity, the fragment comprising eight contiguous amino acids of SEQ ID NO:40;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:39.

Preferably, such polynucleotide comprises the nucleotide sequence of SEQ ID NO:39 from nucleotide 1 to nucleotide 1662; the nucleotide sequence of SEQ ID NO:39 from nucleotide 301 to nucleotide 1662; the nucleotide sequence of SEQ ID NO:39 from nucleotide 1099 to nucleotide 1279; the nucleotide sequence of the full-length protein coding sequence of clone J218_{—}15 deposited with the ATCC under accession number 98028; or the nucleotide sequence of a mature protein coding sequence of clone J218_{—}15 deposited with the ATCC under accession number 98028. In other preferred embodiments, the polynucleotide encodes the full-length or a mature protein encoded by the cDNA insert of clone J218_{—}15 deposited with the ATCC under accession number 98028. In yet other preferred embodiments, the present invention provides a polynucleotide encoding a protein comprising the amino acid sequence of SEQ ID NO:40 from amino acid 367 to amino acid 426. In further preferred embodiments, the present invention provides a polynucleotide encoding a protein comprising a fragment of the amino acid sequence of SEQ ID NO:40 having biological activity, the fragment preferably comprising eight (more preferably twenty, most preferably thirty) contiguous amino acids of SEQ ID NO:40, or a polynucleotide encoding a protein comprising a fragment of the amino acid sequence of SEQ ID NO:40 having biological activity, the fragment comprising the amino acid sequence from amino acid 272 to amino acid 281 of SEQ ID NO:40.

Other embodiments provide the gene corresponding to the cDNA sequence of SEQ ID NO:39.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:39, but excluding the poly(A) tail at the 3′ end of SEQ ID NO:39; and
      (ab) the nucleotide sequence of the cDNA insert of clone J218_{—}15 deposited with the ATCC under accession number 98028;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:39, but excluding the poly(A) tail at the 3′ end of SEQ ID NO:39; and
      (bb) the nucleotide sequence of the cDNA insert of clone J218_{—}15 deposited with the ATCC under accession number 98028;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:39, and extending contiguously from a nucleotide sequence corresponding to the 5′ end of SEQ ID NO:39 to a nucleotide sequence corresponding to the 3′ end of SEQ ID NO:39, but excluding the poly(A) tail at the 3′ end of SEQ ID NO:39. Also preferably the polynucleotide isolated according to the above process comprises a nucleotide sequence corresponding to the cDNA sequence of SEQ ID NO:39 from nucleotide 1 to nucleotide 1662, and extending contiguously from a nucleotide sequence corresponding to the 5′ end of said sequence of SEQ ID NO:39 from nucleotide 1 to nucleotide 1662, to a nucleotide sequence corresponding to the 3′ end of said sequence of SEQ ID NO:39 from nucleotide 1 to nucleotide 1662. Also preferably the polynucleotide isolated according to the above process comprises a nucleotide sequence corresponding to the cDNA sequence of SEQ ID NO:39 from nucleotide 301 to nucleotide 1662, and extending contiguously from a nucleotide sequence corresponding to the 5′ end of said sequence of SEQ ID NO:39 from nucleotide 301 to nucleotide 1662, to a nucleotide sequence corresponding to the 3′ end of said sequence of SEQ ID NO:39 from nucleotide 301 to nucleotide 1662. Also preferably the polynucleotide isolated according to the above process comprises a nucleotide sequence corresponding to the cDNA sequence of SEQ ID NO:39 from nucleotide 1099 to nucleotide 1279, and extending contiguously from a nucleotide sequence corresponding to the 5′ end of said sequence of SEQ ID NO:39 from nucleotide 1099 to nucleotide 1279, to a nucleotide sequence corresponding to the 3′ end of said sequence of SEQ ID NO:39 from nucleotide 1099 to nucleotide 1279.

In other embodiments, the present invention provides a composition comprising a protein, wherein said protein comprises an amino acid sequence selected from the group consisting of:
(a) the amino acid sequence of SEQ ID NO:40;
(b) the amino acid sequence of SEQ ID NO:40 from amino acid 367 to amino acid 426;
(c) a fragment of the amino acid sequence of SEQ ID NO:40, the fragment comprising eight contiguous amino acids of SEQ ID NO:40; and
(d) the amino acid sequence encoded by the cDNA insert of clone J218_{—}15 deposited with the ATCC under accession number 98028;
   the protein being substantially free from other mammalian proteins. Preferably such protein comprises the amino acid sequence of SEQ ID NO:40 or the amino acid sequence of SEQ ID NO:40 from amino acid 367 to amino acid 426. In further preferred embodiments, the present invention provides a protein comprising a fragment of the amino acid sequence of SEQ ID NO:40 having biological activity, the fragment preferably comprising eight (more preferably twenty, most preferably thirty) contiguous amino acids of SEQ ID NO:40, or a protein comprising a fragment of the amino acid sequence of SEQ ID NO:40 having biological activity, the fragment comprising the amino acid sequence from amino acid 272 to amino acid 281 of SEQ ID NO:40.

In one embodiment, the present invention provides a composition comprising an isolated polynucleotide selected from the group consisting of:

accession number 98028;
(d) a polynucleotide encoding the full-length protein encoded by the cDNA insert of clone M8_{—}2 deposited with the ATCC under accession number 98028;
(e) a polynucleotide comprising the nucleotide sequence of a mature protein coding sequence of clone M8_{—}2 deposited with the ATCC under accession number 98028;
(f) a polynucleotide encoding a mature protein encoded by the cDNA insert of clone M8_{—}2 deposited with the ATCC under accession number 98028;
(g) a polynucleotide encoding a protein comprising the amino acid sequence of SEQ ID NO:42;
(h) a polynucleotide encoding a protein comprising a fragment of the amino acid sequence of SEQ ID NO:42 having biological activity, the fragment comprising eight contiguous amino acids of SEQ ID NO:42;
(i) a polynucleotide which is an allelic variant of a polynucleotide of (a)-(f) above;
(j) a polynucleotide which encodes a species homologue of the protein of (g) or (h) above;
(k) a polynucleotide that hybridizes under stringent conditions to any one of the polynucleotides specified in (a)-(h); and
(l) a polynucleotide that hybridizes under stringent conditions to any one of the polynucleotides specified in (a)-(h) and that has a length that is at least 25% of the length of SEQ ID NO:41.

Preferably, such polynucleotide comprises the nucleotide sequence of SEQ ID NO:41 from nucleotide 3 to nucleotide 494; the nucleotide sequence of the full-length protein coding sequence of clone M8_{—}2 deposited with the ATCC under accession number 98028; or the nucleotide sequence of a mature protein coding sequence of clone M8_{—}2 deposited with the ATCC under accession number 98028. In other preferred embodiments, the polynucleotide encodes the full-length or a mature protein encoded by the cDNA insert of clone M8_{—}2 deposited with the ATCC under accession number 98028.

In further preferred embodiments, the present invention provides a polynucleotide encoding a protein comprising a fragment of the amino acid sequence of SEQ ID NO:42 having biological activity, the fragment preferably comprising eight (more preferably twenty, most preferably thirty) contiguous amino acids of SEQ ID NO:42, or a polynucleotide encoding a protein comprising a fragment of the amino acid sequence of SEQ ID NO:42 having biological activity, the fragment comprising the amino acid sequence from amino acid 77 to amino acid 86 of SEQ ID NO:42.

Other embodiments provide the gene corresponding to the cDNA sequence of SEQ ID NO:41.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:41, but excluding the poly(A) tail at the 3′ end of SEQ ID NO:41; and
      (ab) the nucleotide sequence of the cDNA insert of clone M8_{—}2 deposited with the ATCC under accession number 98028;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:41, but excluding the poly(A) tail at the 3′ end of SEQ ID NO:41; and
      (bb) the nucleotide sequence of the cDNA insert of clone M8_{—}2 deposited with the ATCC under accession number 98028;
   (ii) hybridizing said primer(s) to human genomic DNA in conditions at least as stringent as 4×SSC at 50 degrees C.;
   (ii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:41, and extending contiguously from a nucleotide sequence corresponding to the 5′ end of SEQ ID NO:41 to a nucleotide sequence corresponding to the 3′ end of SEQ ID NO:41, but excluding the poly(A) tail at the 3′ end of SEQ ID NO:41. Also preferably the polynucleotide isolated according to the above process comprises a nucleotide sequence corresponding to the cDNA sequence of SEQ ID NO:41 from nucleotide 3 to nucleotide 494, and extending contiguously from a nucleotide sequence corresponding to the 5′ end of said sequence of SEQ ID NO:41 from nucleotide 3 to nucleotide 494, to a nucleotide sequence corresponding to the 3′ end of said sequence of SEQ ID NO:41 from nucleotide 3 to nucleotide 494.

In other embodiments, the present invention provides a composition comprising a protein, wherein said protein comprises an amino acid sequence selected from the group consisting of:
(a) the amino acid sequence of SEQ ID NO:42;
(b) a fragment of the amino acid sequence of SEQ ID NO:42, the fragment comprising eight contiguous amino acids of SEQ ID NO:42; and
(c) the amino acid sequence encoded by the cDNA insert of clone M8_{—}2 deposited with the ATCC under accession number 98028;
   the protein being substantially free from other mammalian proteins. Preferably such protein comprises the amino acid sequence of SEQ ID NO:42. In further preferred embodiments, the present invention provides a protein comprising a fragment of the amino acid sequence of SEQ ID NO:42 having biological activity, the fragment preferably comprising eight (more preferably twenty, most preferably thirty) contiguous amino acids of SEQ ID NO:42, or a protein comprising a fragment of the amino acid sequence of SEQ ID NO:42 having biological activity, the fragment comprising the amino acid sequence from amino acid 77 to amino acid 86 of SEQ ID NO:42.

In one embodiment, the present invention provides a composition comprising an isolated polynucleotide selected from the group consisting of:
(a) a polynucleotide comprising the nucleotide sequence of SEQ ID NO:43;
(b) a polynucleotide comprising the nucleotide sequence of SEQ ID NO:43 from nucleotide 68 to nucleotide 235;
(c) a polynucleotide comprising the nucleotide sequence of SEQ ID NO:43 from nucleotide 161 to nucleotide 235;
(d) a polynucleotide comprising the nucleotide sequence of SEQ ID NO:43 from nucleotide 1 to nucleotide 212;
(e) a polynucleotide comprising the nucleotide sequence of the full-length protein coding sequence of clone M97_{—}2 deposited with the ATCC under accession number 98028;
(f) a polynucleotide encoding the full-length protein encoded by the cDNA insert of clone M97_{—}2 deposited with the ATCC under accession number 98028;
(g) a polynucleotide comprising the nucleotide sequence of a mature protein coding sequence of clone M97_{—}2 deposited with the ATCC under accession number 98028;
(h) a polynucleotide encoding a mature protein encoded by the cDNA insert of clone M97_{—}2 deposited with the ATCC under accession number 98028;
(i) a polynucleotide encoding a protein comprising the amino acid sequence of SEQ ID NO:44;
(j) a polynucleotide encoding a protein comprising a fragment of the amino acid sequence of SEQ ID NO:44 having biological activity, the fragment comprising eight contiguous amino acids of SEQ ID NO:44;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:43.

Preferably, such polynucleotide comprises the nucleotide sequence of SEQ ID NO:43 from nucleotide 68 to nucleotide 235; the nucleotide sequence of SEQ ID NO:43 from nucleotide 161 to nucleotide 235; the nucleotide sequence of SEQ ID NO:43 from nucleotide 1 to nucleotide 212; the nucleotide sequence of the full-length protein coding sequence of clone M97_{—}2 deposited with the ATCC under accession number 98028; or the nucleotide sequence of a mature protein coding sequence of clone M97_{—}2 deposited with the ATCC under accession number 98028. In other preferred embodiments, the polynucleotide encodes the full-length or a mature protein encoded by the cDNA insert of clone M97_{—}2 deposited with the ATCC under accession number 98028. In yet other preferred embodiments, the present invention provides a polynucleotide encoding a protein comprising the amino acid sequence of SEQ ID NO:44 from amino acid 1 to amino acid 48. In further preferred embodiments, the present invention provides a polynucleotide encoding a protein comprising a fragment of the amino acid sequence of SEQ ID NO:44 having biological activity, the fragment preferably comprising eight (more preferably twenty, most preferably thirty) contiguous amino acids of SEQ ID NO:44, or a polynucleotide encoding a protein comprising a fragment of the amino acid sequence of SEQ ID NO:44 having biological activity, the fragment comprising the amino acid sequence from amino acid 23 to amino acid 32 of SEQ ID NO:44.

Other embodiments provide the gene corresponding to the cDNA sequence of SEQ ID NO:43.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:43, but excluding the poly(A) tail at the 3′ end of SEQ ID NO:43; and
      (ab) the nucleotide sequence of the cDNA insert of clone M97_{—}2 deposited with the ATCC under accession number 98028;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:43, but excluding the poly(A) tail at the 3′ end of SEQ ID NO:43; and
      (bb) the nucleotide sequence of the cDNA insert of clone M97_{—}2 deposited with the ATCC under accession number 98028;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:43, and extending contiguously from a nucleotide sequence corresponding to the 5′ end of SEQ ID NO:43 to a nucleotide sequence corresponding to the 3′ end of SEQ ID NO:43, but excluding the poly(A) tail at the 3′ end of SEQ ID NO:43. Also preferably the polynucleotide isolated according to the above process comprises a nucleotide sequence corresponding to the cDNA sequence of SEQ ID NO:43 from nucleotide 68 to nucleotide 235, and extending contiguously from a nucleotide sequence corresponding to the 5′ end of said sequence of SEQ ID NO:43 from nucleotide 68 to nucleotide 235, to a nucleotide sequence corresponding to the 3′ end of said sequence of SEQ ID NO:43 from nucleotide 68 to nucleotide 235. Also preferably the polynucleotide isolated according to the above process comprises a nucleotide sequence corresponding to the cDNA sequence of SEQ ID NO:43 from nucleotide 161 to nucleotide 235, and extending contiguously from a nucleotide sequence corresponding to the 5′ end of said sequence of SEQ ID NO:43 from nucleotide 161 to nucleotide 235, to a nucleotide sequence corresponding to the 3′ end of said sequence of SEQ ID NO:43 from nucleotide 161 to nucleotide 235. Also preferably the polynucleotide isolated according to the above process comprises a nucleotide sequence corresponding to the cDNA sequence of SEQ ID NO:43 from nucleotide 1 to nucleotide 212, and extending contiguously from a nucleotide sequence corresponding to the 5′ end of said sequence of SEQ ID NO:43 from nucleotide 1 to nucleotide 212, to a nucleotide sequence corresponding to the 3′ end of said sequence of SEQ ID NO:43 from nucleotide 1 to nucleotide 212.

In other embodiments, the present invention provides a composition comprising a protein, wherein said protein comprises an amino acid sequence selected from the group consisting of:
(a) the amino acid sequence of SEQ ID NO:44;
(b) the amino acid sequence of SEQ ID NO:44 from amino acid 1 to amino acid 48;
(c) a fragment of the amino acid sequence of SEQ ID NO:44, the fragment comprising eight contiguous amino acids of SEQ ID NO:44; and
(d) the amino acid sequence encoded by the cDNA insert of clone M97_{—}2 deposited with the ATCC under accession number 98028;
   the protein being substantially free from other mammalian proteins. Preferably such protein comprises the amino acid sequence of SEQ ID NO:44 or the amino acid sequence of SEQ ID NO:44 from amino acid 1 to amino acid 48. In further preferred embodiments, the present invention provides a protein comprising a fragment of the amino acid sequence of SEQ ID NO:44 having biological activity, the fragment preferably comprising eight (more preferably twenty, most preferably thirty) contiguous amino acids of SEQ ID NO:44, or a protein comprising a fragment of the amino acid sequence of SEQ ID NO:44 having biological activity, the fragment comprising the amino acid sequence from amino acid 23 to amino acid 32 of SEQ ID NO:44.

In one embodiment, the present invention provides a composition comprising an isolated polynucleotide selected from the group consisting of:
(a) a polynucleotide comprising the nucleotide sequence of SEQ ID NO:45;
(b) a polynucleotide comprising the nucleotide sequence of SEQ ID NO:45 from nucleotide 10 to nucleotide 549;
(c) a polynucleotide comprising the nucleotide sequence of SEQ ID NO:45 from nucleotide 124 to nucleotide 549;
(d) a polynucleotide comprising the nucleotide sequence of SEQ ID NO:45 from nucleotide 279 to nucleotide 537;
(e) a polynucleotide comprising the nucleotide sequence of the full-length protein coding sequence of clone O238_{—}1 deposited with the ATCC under accession number 98028;
(f) a polynucleotide encoding the full-length protein encoded by the cDNA insert of clone O238_{—}1 deposited with the ATCC under accession number 98028;
(g) a polynucleotide comprising the nucleotide sequence of a mature protein coding sequence of clone O238_{—}1 deposited with the ATCC under accession number 98028;
(h) a polynucleotide encoding a mature protein encoded by the cDNA insert of clone O238_{—}1 deposited with the ATCC under accession number 98028;
(i) a polynucleotide encoding a protein comprising the amino acid sequence of SEQ ID NO:46;
(j) a polynucleotide encoding a protein comprising a fragment of the amino acid sequence of SEQ ID NO:46 having biological activity, the fragment comprising eight contiguous amino acids of SEQ ID NO:46;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:45.

Preferably, such polynucleotide comprises the nucleotide sequence of SEQ ID NO:45 from nucleotide 10 to nucleotide 549; the nucleotide sequence of SEQ ID NO:45 from nucleotide 124 to nucleotide 549; the nucleotide sequence of SEQ ID NO:45 from nucleotide 279 to nucleotide 537; the nucleotide sequence of the full-length protein coding sequence of clone O238_{—}1 deposited with the ATCC under accession number 98028; or the nucleotide sequence of a mature protein coding sequence of clone O238_{—}1 deposited with the ATCC under accession number 98028. In other preferred embodiments, the polynucleotide encodes the full-length or a mature protein encoded by the cDNA insert of clone O238_{—}1 deposited with the ATCC under accession number 98028. In yet other preferred embodiments, the present invention provides a polynucleotide encoding a protein comprising the amino acid sequence of SEQ ID NO:46 from amino acid 91 to amino acid 176. In further preferred embodiments, the present invention provides a polynucleotide encoding a protein comprising a fragment of the amino acid sequence of SEQ ID NO:46 having biological activity, the fragment preferably comprising eight (more preferably twenty, most preferably thirty) contiguous amino acids of SEQ ID NO:46, or a polynucleotide encoding a protein comprising a fragment of the amino acid sequence of SEQ ID NO:46 having biological activity, the fragment comprising the amino acid sequence from amino acid 85 to amino acid 94 of SEQ ID NO:46.

Other embodiments provide the gene corresponding to the cDNA sequence of SEQ ID NO:45.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:45; and
      (ab) the nucleotide sequence of the cDNA insert of clone O238_{—}1 deposited with the ATCC under accession number 98028;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:45; and
      (bb) the nucleotide sequence of the cDNA insert of clone O238_{—}1 deposited with the ATCC under accession number 98028;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:45, and extending contiguously from a nucleotide sequence corresponding to the 5′ end of SEQ ID NO:45 to a nucleotide sequence corresponding to the 3′ end of SEQ ID NO:45. Also preferably the polynucleotide isolated according to the above process comprises a nucleotide sequence corresponding to the cDNA sequence of SEQ ID NO:45 from nucleotide 10 to nucleotide 549, and extending contiguously from a nucleotide sequence corresponding to the 5′ end of said sequence of SEQ ID NO:45 from nucleotide 10 to nucleotide 549, to a nucleotide sequence corresponding to the 3′ end of said sequence of SEQ ID NO:45 from nucleotide 10 to nucleotide 549. Also preferably the polynucleotide isolated according to the above process comprises a nucleotide sequence corresponding to the cDNA sequence of SEQ ID NO:45 from nucleotide 124 to nucleotide 549, and extending contiguously from a nucleotide sequence corresponding to the 5′ end of said sequence of SEQ ID NO:45 from nucleotide 124 to nucleotide 549, to a nucleotide sequence corresponding to the 3′ end of said sequence of SEQ ID NO:45 from nucleotide 124 to nucleotide 549. Also preferably the polynucleotide isolated according to the above process comprises a nucleotide sequence corresponding to the cDNA sequence of SEQ ID NO:45 from nucleotide 279 to nucleotide 537, and extending contiguously from a nucleotide sequence corresponding to the 5′ end of said sequence of SEQ ID NO:45 from nucleotide 279 to nucleotide 537, to a nucleotide sequence corresponding to the 3′ end of said sequence of SEQ ID NO:45 from nucleotide 279 to nucleotide 537.

In other embodiments, the present invention provides a composition comprising a protein, wherein said protein comprises an amino acid sequence selected from the group consisting of:
(a) the amino acid sequence of SEQ ID NO:46;
(b) the amino acid sequence of SEQ ID NO:46 from amino acid 91 to amino acid 176;
(c) a fragment of the amino acid sequence of SEQ ID NO:46, the fragment comprising eight contiguous amino acids of SEQ ID NO:46; and
(d) the amino acid sequence encoded by the cDNA insert of clone O238_{—}1 deposited with the ATCC under accession number 98028;
   the protein being substantially free from other mammalian proteins. Preferably such protein comprises the amino acid sequence of SEQ ID NO:46 or the amino acid sequence of SEQ ID NO:46 from amino acid 91 to amino acid 176. In further preferred embodiments, the present invention provides a protein comprising a fragment of the amino acid sequence of SEQ ID NO:46 having biological activity, the fragment preferably comprising eight (more preferably twenty, most preferably thirty) contiguous amino acids of SEQ ID NO:46, or a protein comprising a fragment of the amino acid sequence of SEQ ID NO:46 having biological activity, the fragment comprising the amino acid sequence from amino acid 85 to amino acid 94 of SEQ ID NO:46.

In one embodiment, the present invention provides a composition comprising an isolated polynucleotide selected from the group consisting of:
(a) a polynucleotide comprising the nucleotide sequence of SEQ ID NO:47;
(b) a polynucleotide comprising the nucleotide sequence of SEQ ID NO:47 from nucleotide 40 to nucleotide 606;
(c) a polynucleotide comprising the nucleotide sequence of SEQ ID NO:47 from nucleotide 178 to nucleotide 606;
(d) a polynucleotide comprising the nucleotide sequence of SEQ ID NO:47 from nucleotide 25 to nucleotide 287;
(e) a polynucleotide comprising the nucleotide sequence of the full-length protein coding sequence of clone S185_{—}2 deposited with the ATCC under accession number 98028;
(f) a polynucleotide encoding the full-length protein encoded by the cDNA insert of clone S185_{—}2 deposited with the ATCC under accession number 98028;
(g) a polynucleotide comprising the nucleotide sequence of a mature protein coding sequence of clone S185_{—}2 deposited with the ATCC under accession number 98028;
(h) a polynucleotide encoding a mature protein encoded by the cDNA insert of clone S185_{—}2 deposited with the ATCC under accession number 98028;
(i) a polynucleotide encoding a protein comprising the amino acid sequence of SEQ ID NO:48;
(j) a polynucleotide encoding a protein comprising a fragment of the amino acid sequence of SEQ ID NO:48 having biological activity, the fragment comprising eight contiguous amino acids of SEQ ID NO:48;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:47.

Preferably, such polynucleotide comprises the nucleotide sequence of SEQ ID NO:47 from nucleotide 40 to nucleotide 606; the nucleotide sequence of SEQ ID NO:47 from nucleotide 178 to nucleotide 606; the nucleotide sequence of SEQ ID NO:47 from nucleotide 25 to nucleotide 287; the nucleotide sequence of the full-length protein coding sequence of clone S185_{—}2 deposited with the ATCC under accession number 98028; or the nucleotide sequence of a mature protein coding sequence of clone S185_{—}2 deposited with the ATCC under accession number 98028. In other preferred embodiments, the polynucleotide encodes the full-length or a mature protein encoded by the cDNA insert of clone S185_{—}2 deposited with the ATCC under accession number 98028. In yet other preferred embodiments, the present invention provides a polynucleotide encoding a protein comprising the amino acid sequence of SEQ ID NO:48 from amino acid 1 to amino acid 42, or a polynucleotide encoding a protein comprising the amino acid sequence of SEQ ID NO:48 from amino acid 1 to amino acid 85. In further preferred embodiments, the present invention provides a polynucleotide encoding a protein comprising a fragment of the amino acid sequence of SEQ ID NO:48 having biological activity, the fragment preferably comprising eight (more preferably twenty, most preferably thirty) contiguous amino acids of SEQ ID NO:48, or a polynucleotide encoding a protein comprising a fragment of the amino acid sequence of SEQ ID NO:48 having biological activity, the fragment comprising the amino acid sequence from amino acid 89 to amino acid 98 of SEQ ID NO:48.

Other embodiments provide the gene corresponding to the cDNA sequence of SEQ ID NO:47.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:47, but excluding the poly(A) tail at the 3′ end of SEQ ID NO:47; and
      (ab) the nucleotide sequence of the cDNA insert of clone S185_{—}2 deposited with the ATCC under accession number 98028;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:47, but excluding the poly(A) tail at the 3′ end of SEQ ID NO:47; and
      (bb) the nucleotide sequence of the cDNA insert of clone S185_{—}2 deposited with the ATCC under accession number 98028;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:47, and extending contiguously from a nucleotide sequence corresponding to the 5′ end of SEQ ID NO:47 to a nucleotide sequence corresponding to the 3′ end of SEQ ID NO:47, but excluding the poly(A) tail at the 3′ end of SEQ ID NO:47. Also preferably the polynucleotide isolated according to the above process comprises a nucleotide sequence corresponding to the cDNA sequence of SEQ ID NO:47 from nucleotide 40 to nucleotide 606, and extending contiguously from a nucleotide sequence corresponding to the 5′ end of said sequence of SEQ ID NO:47 from nucleotide 40 to nucleotide 606, to a nucleotide sequence corresponding to the 3′ end of said sequence of SEQ ID NO:47 from nucleotide 40 to nucleotide 606. Also preferably the polynucleotide isolated according to the above process comprises a nucleotide sequence corresponding to the cDNA sequence of SEQ ID NO:47 from nucleotide 178 to nucleotide 606, and extending contiguously from a nucleotide sequence corresponding to the 5′ end of said sequence of SEQ ID NO:47 from nucleotide 178 to nucleotide 606, to a nucleotide sequence corresponding to the 3′ end of said sequence of SEQ ID NO:47 from nucleotide 178 to nucleotide 606. Also preferably the polynucleotide isolated according to the above process comprises a nucleotide sequence corresponding to the cDNA sequence of SEQ ID NO:47 from nucleotide 25 to nucleotide 287, and extending contiguously from a nucleotide sequence corresponding to the 5′ end of said sequence of SEQ ID NO:47 from nucleotide 25 to nucleotide 287, to a nucleotide sequence corresponding to the 3′ end of said sequence of SEQ ID NO:47 from nucleotide 25 to nucleotide 287.

In other embodiments, the present invention provides a composition comprising a protein, wherein said protein comprises an amino acid sequence selected from the group consisting of:
(a) the amino acid sequence of SEQ ID NO:48;
(b) the amino acid sequence of SEQ ID NO:48 from amino acid 1 to amino acid 42;
(c) a fragment of the amino acid sequence of SEQ ID NO:48, the fragment comprising eight contiguous amino acids of SEQ ID NO:48; and
(d) the amino acid sequence encoded by the cDNA insert of clone S185_{—}2 deposited with the ATCC under accession number 98028;
   the protein being substantially free from other mammalian proteins. Preferably such protein comprises the amino acid sequence of SEQ ID NO:48, the amino acid sequence of SEQ ID NO:48 from amino acid 1 to amino acid 42, or the amino acid sequence of SEQ ID NO:48 from amino acid 1 to amino acid 85. In further preferred embodiments, the present invention provides a protein comprising a fragment of the amino acid sequence of SEQ ID NO:48 having biological activity, the fragment preferably comprising eight (more preferably twenty, most preferably thirty) contiguous amino acids of SEQ ID NO:48, or a protein comprising a fragment of the amino acid sequence of SEQ ID NO:48 having biological activity, the fragment comprising the amino acid sequence from amino acid 89 to amino acid 98 of SEQ ID NO:48.

In one embodiment, the present invention provides a composition comprising an isolated polynucleotide selected from the group consisting of:
(a) a polynucleotide comprising the nucleotide sequence of SEQ ID NO:49;
(b) a polynucleotide comprising the nucleotide sequence of SEQ ID NO:49 from nucleotide 75 to nucleotide 353;
(c) a polynucleotide comprising the nucleotide sequence of SEQ ID NO:49 from nucleotide 132 to nucleotide 353;
(d) a polynucleotide comprising the nucleotide sequence of SEQ ID NO:49 from nucleotide 10 to nucleotide 304;
(e) a polynucleotide comprising the nucleotide sequence of the full-length protein coding sequence of clone AJ147_{—}1 deposited with the ATCC under accession number 98076;
(f) a polynucleotide encoding the full-length protein encoded by the cDNA insert of clone AJ147_{—}1 deposited with the ATCC under accession number 98076;
(g) a polynucleotide comprising the nucleotide sequence of a mature protein coding sequence of clone AJ147_{—}1 deposited with the ATCC under accession number 98076;
(h) a polynucleotide encoding a mature protein encoded by the cDNA insert of clone AJ147_{—}1 deposited with the ATCC under accession number 98076;
(i) a polynucleotide encoding a protein comprising the amino acid sequence of SEQ ID NO:50;
(j) a polynucleotide encoding a protein comprising a fragment of the amino acid sequence of SEQ ID NO:50 having biological activity, the fragment comprising eight contiguous amino acids of SEQ ID NO:50;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:49.

Preferably, such polynucleotide comprises the nucleotide sequence of SEQ ID NO:49 from nucleotide 75 to nucleotide 353; the nucleotide sequence of SEQ ID NO:49 from nucleotide 132 to nucleotide 353; the nucleotide sequence of SEQ ID NO:49 from nucleotide 10 to nucleotide 304; the nucleotide sequence of the full-length protein coding sequence of clone AJ147_{—}1 deposited with the ATCC under accession number 98076; or the nucleotide sequence of a mature protein coding sequence of clone AJ147_{—}1 deposited with the ATCC under accession number 98076. In other preferred embodiments, the polynucleotide encodes the full-length or a mature protein encoded by the cDNA insert of clone AJ147_{—}1 deposited with the ATCC under accession number 98076. In yet other preferred embodiments, the present invention provides a polynucleotide encoding a protein comprising the amino acid sequence of SEQ ID NO:50 from amino acid 1 to amino acid 57. In further preferred embodiments, the present invention provides a polynucleotide encoding a protein comprising a fragment of the amino acid sequence of SEQ ID NO:50 having biological activity, the fragment preferably comprising eight (more preferably twenty, most preferably thirty) contiguous amino acids of SEQ ID NO:50, or a polynucleotide encoding a protein comprising a fragment of the amino acid sequence of SEQ ID NO:50 having biological activity, the fragment comprising the amino acid sequence from amino acid 41 to amino acid 50 of SEQ ID NO:50.

Other embodiments provide the gene corresponding to the cDNA sequence of SEQ ID NO:49.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:49, but excluding the poly(A) tail at the 3′ end of SEQ ID NO:49; and
      (ab) the nucleotide sequence of the cDNA insert of clone AJ147_{—}1 deposited with the ATCC under accession number 98076;
   (ii) hybridizing said probe(s) to human genomic DNA in conditions at least as stringent as 4×SSC at 50 degrees C.; and
   (ii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:49, but excluding the poly(A) tail at the 3′ end of SEQ ID NO:49; and
      (bb) the nucleotide sequence of the cDNA insert of clone AJ147_{—}1 deposited with the ATCC under accession number 98076;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:49, and extending contiguously from a nucleotide sequence corresponding to the 5′ end of SEQ ID NO:49 to a nucleotide sequence corresponding to the 3′ end of SEQ ID NO:49, but excluding the poly(A) tail at the 3′ end of SEQ ID NO:49. Also preferably the polynucleotide isolated according to the above process comprises a nucleotide sequence corresponding to the cDNA sequence of SEQ ID NO:49 from nucleotide 75 to nucleotide 353, and extending contiguously from a nucleotide sequence corresponding to the 5′ end of said sequence of SEQ ID NO:49 from nucleotide 75 to nucleotide 353, to a nucleotide sequence corresponding to the 3′ end of said sequence of SEQ ID NO:49 from nucleotide 75 to nucleotide 353. Also preferably the polynucleotide isolated according to the above process comprises a nucleotide sequence corresponding to the cDNA sequence of SEQ ID NO:49 from nucleotide 132 to nucleotide 353, and extending contiguously from a nucleotide sequence corresponding to the 5′ end of said sequence of SEQ ID NO:49 from nucleotide 132 to nucleotide 353, to a nucleotide sequence corresponding to the 3′ end of said sequence of SEQ ID NO:49 from nucleotide 132 to nucleotide 353. Also preferably the polynucleotide isolated according to the above process comprises a nucleotide sequence corresponding to the cDNA sequence of SEQ ID NO:49 from nucleotide 10 to nucleotide 304, and extending contiguously from a nucleotide sequence corresponding to the 5′ end of said sequence of SEQ ID NO:49 from nucleotide 10 to nucleotide 304, to a nucleotide sequence corresponding to the 3′ end of said sequence of SEQ ID NO:49 from nucleotide 10 to nucleotide 304.

In other embodiments, the present invention provides a composition comprising a protein, wherein said protein comprises an amino acid sequence selected from the group consisting of:
(a) the amino acid sequence of SEQ ID NO:50;
(b) the amino acid sequence of SEQ ID NO:50 from amino acid 1 to amino acid 57;
(c) a fragment of the amino acid sequence of SEQ ID NO:50, the fragment comprising eight contiguous amino acids of SEQ ID NO:50; and
(d) the amino acid sequence encoded by the cDNA insert of clone AJ147_{—}1 deposited with the ATCC under accession number 98076;
   the protein being substantially free from other mammalian proteins. Preferably such protein comprises the amino acid sequence of SEQ ID NO:50 or the amino acid sequence of SEQ ID NO:50 from amino acid 1 to amino acid 57. In further preferred embodiments, the present invention provides a protein comprising a fragment of the amino acid sequence of SEQ ID NO:50 having biological activity, the fragment preferably comprising eight (more preferably twenty, most preferably thirty) contiguous amino acids of SEQ ID NO:50, or a protein comprising a fragment of the amino acid sequence of SEQ ID NO:50 having biological activity, the fragment comprising the amino acid sequence from amino acid 41 to amino acid 50 of SEQ ID NO:50.

In one embodiment, the present invention provides a composition comprising an isolated polynucleotide selected from the group consisting of:
(a) a polynucleotide comprising the nucleotide sequence of SEQ ID NO:51;
(b) a polynucleotide comprising the nucleotide sequence of SEQ ID NO:51 from nucleotide 54 to nucleotide 344;
(c) a polynucleotide comprising the nucleotide sequence of SEQ ID NO:51 from nucleotide 108 to nucleotide 344;
(d) a polynucleotide comprising the nucleotide sequence of SEQ ID NO:51 from nucleotide 98 to nucleotide 268;
(e) a polynucleotide comprising the nucleotide sequence of the full-length protein coding sequence of clone AM262_{—}11 deposited with the ATCC under accession number 98076;
(f) a polynucleotide encoding the full-length protein encoded by the cDNA insert of clone AM262_{—}11 deposited with the ATCC under accession number 98076;
(g) a polynucleotide comprising the nucleotide sequence of a mature protein coding sequence of clone AM262_{—}11 deposited with the ATCC under accession number 98076;
(h) a polynucleotide encoding a mature protein encoded by the cDNA insert of clone AM262_{—}11 deposited with the ATCC under accession number 98076;
(i) a polynucleotide encoding a protein comprising the amino acid sequence of SEQ ID NO:52;
(j) a polynucleotide encoding a protein comprising a fragment of the amino acid sequence of SEQ ID NO:52 having biological activity, the fragment comprising eight contiguous amino acids of SEQ ID NO:52;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:51.

Preferably, such polynucleotide comprises the nucleotide sequence of SEQ ID NO:51 from nucleotide 54 to nucleotide 344; the nucleotide sequence of SEQ ID NO:51 from nucleotide 108 to nucleotide 344; the nucleotide sequence of SEQ ID NO:51 from nucleotide 98 to nucleotide 268; the nucleotide sequence of the full-length protein coding sequence of clone AM262_{—}11 deposited with the ATCC under accession number 98076; or the nucleotide sequence of a mature protein coding sequence of clone AM262_{—}11 deposited with the ATCC under accession number 98076. In other preferred embodiments, the polynucleotide encodes the full-length or a mature protein encoded by the cDNA insert of clone AM262_{—}11 deposited with the ATCC under accession number 98076. In yet other preferred embodiments, the present invention provides a polynucleotide encoding a protein comprising the amino add sequence of SEQ ID NO:52 from amino acid 16 to amino acid 82. In further preferred embodiments, the present invention provides a polynucleotide encoding a protein comprising a fragment of the amino acid sequence of SEQ ID NO:52 having biological activity, the fragment preferably comprising eight (more preferably twenty, most preferably thirty) contiguous amino acids of SEQ ID NO:52, or a polynucleotide encoding a protein comprising a fragment of the amino acid sequence of SEQ ID NO:52 having biological activity, the fragment comprising the amino acid sequence from amino acid 43 to amino acid 52 of SEQ ID NO:52.

Other embodiments provide the gene corresponding to the cDNA sequence of SEQ ID NO:51.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:51, but excluding the poly(A) tail at the 3′ end of SEQ ID NO:51; and
      (ab) the nucleotide sequence of the cDNA insert of clone AM262_{—}11 deposited with the ATCC under accession number 98076;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:51, but excluding the poly(A) tail at the 3′ end of SEQ ID NO:51; and
      (bb) the nucleotide sequence of the cDNA insert of clone AM262_{—}11 deposited with the ATCC under accession number 98076;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:51, and extending contiguously from a nucleotide sequence corresponding to the 5′ end of SEQ ID NO:51 to a nucleotide sequence corresponding to the 3′ end of SEQ ID NO:51, but excluding the poly(A) tail at the 3′ end of SEQ ID NO:51. Also preferably the polynucleotide isolated according to the above process comprises a nucleotide sequence corresponding to the cDNA sequence of SEQ ID NO:51 from nucleotide 54 to nucleotide 344, and extending contiguously from a nucleotide sequence corresponding to the 5′ end of said sequence of SEQ ID NO:51 from nucleotide 54 to nucleotide 344, to a nucleotide sequence corresponding to the 3′ end of said sequence of SEQ ID NO:51 from nucleotide 54 to nucleotide 344. Also preferably the polynucleotide isolated according to the above process comprises a nucleotide sequence corresponding to the cDNA sequence of SEQ ID NO:51 from nucleotide 108 to nucleotide 344, and extending contiguously from a nucleotide sequence corresponding to the 5′ end of said sequence of SEQ ID NO:51 from nucleotide 108 to nucleotide 344, to a nucleotide sequence corresponding to the 3′ end of said sequence of SEQ ID NO:51 from nucleotide 108 to nucleotide 344. Also preferably the polynucleotide isolated according to the above process comprises a nucleotide sequence corresponding to the cDNA sequence of SEQ ID NO:51 from nucleotide 98 to nucleotide 268, and extending contiguously from a nucleotide sequence corresponding to the 5′ end of said sequence of SEQ ID NO:51 from nucleotide 98 to nucleotide 268, to a nucleotide sequence corresponding to the 3′ end of said sequence of SEQ ID NO:51 from nucleotide 98 to nucleotide 268.

In other embodiments, the present invention provides a composition comprising a protein, wherein said protein comprises an amino acid sequence selected from the group consisting of:
(a) the amino acid sequence of SEQ ID NO:52;
(b) the amino acid sequence of SEQ ID NO:52 from amino acid 16 to amino acid 82;
(c) a fragment of the amino acid sequence of SEQ ID NO:52, the fragment comprising eight contiguous amino acids of SEQ ID NO:52; and
(d) the amino acid sequence encoded by the cDNA insert of clone AM262_{—}11 deposited with the ATCC under accession number 98076;
   the protein being substantially free from other mammalian proteins. Preferably such protein comprises the amino acid sequence of SEQ ID NO:52 or the amino acid sequence of SEQ ID NO:52 from amino acid 16 to amino acid 82. In further preferred embodiments, the present invention provides a protein comprising a fragment of the amino acid sequence of SEQ ID NO:52 having biological activity, the fragment preferably comprising eight (more preferably twenty, most preferably thirty) contiguous amino acids of SEQ ID NO:52, or a protein comprising a fragment of the amino acid sequence of SEQ ID NO:52 having biological activity, the fragment comprising the amino acid sequence from amino acid 43 to amino acid 52 of SEQ ID NO:52.

In one embodiment, the present invention provides a composition comprising an isolated polynucleotide selected from the group consisting of:
(a) a polynucleotide comprising the nucleotide sequence of SEQ ID NO:53;
(b) a polynucleotide comprising the nucleotide sequence of SEQ ID NO:53 from nucleotide 83 to nucleotide 991;
(c) a polynucleotide comprising the nucleotide sequence of SEQ ID NO:53 from nucleotide 198 to nucleotide 421;
(d) a polynucleotide comprising the nucleotide sequence of the full-length protein coding sequence of clone AR28_{—}1 deposited with the ATCC under accession number 98076;
(e) a polynucleotide encoding the full-length protein encoded by the cDNA insert of clone AR28_{—}1 deposited with the ATCC under accession number 98076;
(f) a polynucleotide comprising the nucleotide sequence of a mature protein coding sequence of clone AR28_{—}1 deposited with the ATCC under accession number 98076;
(g) a polynucleotide encoding a mature protein encoded by the cDNA insert of clone AR28_{—}1 deposited with the ATCC under accession number 98076;
(h) a polynucleotide encoding a protein comprising the amino acid sequence of SEQ ID NO:54;
(i) a polynucleotide encoding a protein comprising a fragment of the amino acid sequence of SEQ ID NO:54 having biological activity, the fragment comprising eight contiguous amino acids of SEQ ID NO:54;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:53.

Preferably, such polynucleotide comprises the nucleotide sequence of SEQ ID NO:53 from nucleotide 83 to nucleotide 991; the nucleotide sequence of SEQ ID NO:53 from nucleotide 198 to nucleotide 421; the nucleotide sequence of the full-length protein coding sequence of clone AR28_{—}1 deposited with the ATCC under accession number 98076; or the nucleotide sequence of a mature protein coding sequence of clone AR28_{—}1 deposited with the ATCC under accession number 98076. In other preferred embodiments, the polynucleotide encodes the full-length or a mature protein encoded by the cDNA insert of clone AR28_{—}1 deposited with the ATCC under accession number 98076. In yet other preferred embodiments, the present invention provides a polynucleotide encoding a protein comprising the amino acid sequence of SEQ ID NO:54 from amino acid 40 to amino acid 113. In further preferred embodiments, the present invention provides a polynucleotide encoding a protein comprising a fragment of the amino acid sequence of SEQ ID NO:54 having biological activity, the fragment preferably comprising eight (more preferably twenty, most preferably thirty) contiguous amino acids of SEQ ID NO:54, or a polynucleotide encoding a protein comprising a fragment of the amino acid sequence of SEQ ID NO:54 having biological activity, the fragment comprising the amino acid sequence from amino acid 146 to amino acid 155 of SEQ ID NO:54.

Other embodiments provide the gene corresponding to the cDNA sequence of SEQ ID NO:53.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:53, but excluding the poly(A) tail at the 3′ end of SEQ ID NO:53; and
      (ab) the nucleotide sequence of the cDNA insert of clone AR28_{—}1 deposited with the ATCC under accession number 98076:
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:53, but excluding the poly(A) tail at the 3′ end of SEQ ID NO:53; and
      (bb) the nucleotide sequence of the cDNA insert of clone AR28_{—}1 deposited with the ATCC under accession number 98076;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ. ID NO:53, and extending contiguously from a nucleotide sequence corresponding to the 5′ end of SEQ ID NO:53 to a nucleotide sequence corresponding to the 3′ end of SEQ ID NO:53, but excluding the poly(A) tail at the 3′ end of SEQ ID NO:53. Also preferably the polynucleotide isolated according to the above process comprises a nucleotide sequence corresponding to the cDNA sequence of SEQ ID NO:53 from nucleotide 83 to nucleotide 991, and extending contiguously from a nucleotide sequence corresponding to the 5′ end of said sequence of SEQ ID NO:53 from nucleotide 83 to nucleotide 991, to a nucleotide sequence corresponding to the 3′ end of said sequence of SEQ ID NO:53 from nucleotide 83 to nucleotide 991. Also preferably the polynucleotide isolated according to the above process comprises a nucleotide sequence corresponding to the cDNA sequence of SEQ ID NO:53 from nucleotide 198 to nucleotide 421, and extending contiguously from a nucleotide sequence corresponding to the 5′ end of said sequence of SEQ ID NO:53 from nucleotide 198 to nucleotide 421, to a nucleotide sequence corresponding to the 3′ end of said sequence of SEQ ID NO:53 from nucleotide 198 to nucleotide 421.

In other embodiments, the present invention provides a composition comprising a protein, wherein said protein comprises an amino acid sequence selected from the group consisting of:
(a) the amino acid sequence of SEQ ID NO:54;
(b) the amino acid sequence of SEQ ID NO:54 from amino acid 40 to amino acid 113;
(c) a fragment of the amino acid sequence of SEQ ID NO:54, the fragment comprising eight contiguous amino acids of SEQ ID NO:54; and
(d) the amino acid sequence encoded by the cDNA insert of clone AR28_{—}1 deposited with the ATCC under accession number 98076;
   the protein being substantially free from other mammalian proteins. Preferably such protein comprises the amino acid sequence of SEQ ID NO:54 or the amino acid sequence of SEQ ID NO:54 from amino acid 40 to amino acid 113. In further preferred embodiments, the present invention provides a protein comprising a fragment of the amino acid sequence of SEQ ID NO:54 having biological activity, the fragment preferably comprising eight (more preferably twenty, most preferably thirty) contiguous amino acids of SEQ ID NO:54, or a protein comprising a fragment of the amino acid sequence of SEQ ID NO:54 having biological activity, the fragment comprising the amino acid sequence from amino acid 146 to amino acid 155 of SEQ ID NO:54.

In one embodiment, the present invention provides a composition comprising an isolated polynucleotide selected from the group consisting of:
(a) a polynucleotide comprising the nucleotide sequence of SEQ ID NO:55;
(b) a polynucleotide comprising the nucleotide sequence of SEQ ID NO:55 from nucleotide 610 to nucleotide 849;
(c) a polynucleotide comprising the nucleotide sequence of SEQ ID NO:55 from nucleotide 730 to nucleotide 849;
(d) a polynucleotide comprising the nucleotide sequence of SEQ ID NO:55 from nucleotide 540 to nucleotide 769;
(e) a polynucleotide comprising the nucleotide sequence of the full-length protein coding sequence of clone AS86_{—}1 deposited with the ATCC under accession number 98076;
(f) a polynucleotide encoding the full-length protein encoded by the cDNA insert of clone AS86_{—}1 deposited with the ATCC under accession number 98076;
(g) a polynucleotide comprising the nucleotide sequence of a mature protein coding sequence of clone AS86_{—}1 deposited with the ATCC under accession number 98076;
(h) a polynucleotide encoding a mature protein encoded by the cDNA insert of clone AS86_{—}1 deposited with the ATCC under accession number 98076;
(i) a polynucleotide encoding a protein comprising the amino acid sequence of SEQ ID NO:56;
(j) a polynucleotide encoding a protein comprising a fragment of the amino acid sequence of SEQ ID NO:56 having biological activity, the fragment comprising eight contiguous amino acids of SEQ ID NO:56;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:55.

Preferably, such polynucleotide comprises the nucleotide sequence of SEQ ID NO:55 from nucleotide 610 to nucleotide 849; the nucleotide sequence of SEQ ID NO:55 from nucleotide 730 to nucleotide 849; the nucleotide sequence of SEQ ID NO:55 from nucleotide 540 to nucleotide 769; the nucleotide sequence of the full-length protein coding sequence of clone AS86_{—}1 deposited with the ATCC under accession number 98076; or the nucleotide sequence of a mature protein coding sequence of clone AS86_{—}1 deposited with the ATCC under accession number 98076. In other preferred embodiments, the polynucleotide encodes the full-length or a mature protein encoded by the cDNA insert of clone AS86_{—}1 deposited with the ATCC under accession number 98076. In yet other preferred embodiments, the present invention provides a polynucleotide encoding a protein comprising the amino acid sequence of SEQ ID NO:56 from amino acid 1 to amino acid 53. In further preferred embodiments, the present invention provides a polynucleotide encoding a protein comprising a fragment of the amino acid sequence of SEQ ID NO:56 having biological activity, the fragment preferably comprising eight (more preferably twenty, most preferably thirty) contiguous amino acids of SEQ ID NO:56, or a polynucleotide encoding a protein comprising a fragment of the amino acid sequence of SEQ ID NO:56 having biological activity, the fragment comprising the amino acid sequence from amino acid 35 to amino acid 44 of SEQ ID NO:56.

Other embodiments provide the gene corresponding to the cDNA sequence of SEQ ID NO:55.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:55, but excluding the poly(A) tail at the 3′ end of SEQ ID NO:55; and
      (ab) the nucleotide sequence of the cDNA insert of clone AS86_{—}1 deposited with the ATCC under accession number 98076;
   (ii) hybridizing said probe(s) to human genomic DNA in conditions at least as stringent as 4×SSC at 50 degrees C.; and
   (ii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:55, but excluding the poly(A) tail at the 3′ end of SEQ ID NO:55; and
      (bb) the nucleotide sequence of the cDNA insert of clone AS86_{—}1 deposited with the ATCC under accession number 98076;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:55, and extending contiguously from a nucleotide sequence corresponding to the 5′ end of SEQ ID NO:55 to a nucleotide sequence corresponding to the 3′ end of SEQ ID NO:55, but excluding the poly(A) tail at the 3′ end of SEQ ID NO:55. Also preferably the polynucleotide isolated according to the above process comprises a nucleotide sequence corresponding to the cDNA sequence of SEQ ID NO:55 from nucleotide 610 to nucleotide 849, and extending contiguously from a nucleotide sequence corresponding to the 5′ end of said sequence of SEQ ID NO:55 from nucleotide 610 to nucleotide 849, to a nucleotide sequence corresponding to the 3′ end of said sequence of SEQ ID NO:55 from nucleotide 610 to nucleotide 849. Also preferably the polynucleotide isolated according to the above process comprises a nucleotide sequence corresponding to the cDNA sequence of SEQ ID NO:55 from nucleotide 730 to nucleotide 849, and extending contiguously from a nucleotide sequence corresponding to the 5′ end of said sequence of SEQ ID NO:55 from nucleotide 730 to nucleotide 849, to a nucleotide sequence corresponding to the 3′ end of said sequence of SEQ ID NO:55 from nucleotide 730 to nucleotide 849. Also preferably the polynucleotide isolated according to the above process comprises a nucleotide sequence corresponding to the cDNA sequence of SEQ ID NO:55 from nucleotide 540 to nucleotide 769, and extending contiguously from a nucleotide sequence corresponding to the 5′ end of said sequence of SEQ ID NO:55 from nucleotide 540 to nucleotide 769, to a nucleotide sequence corresponding to the 3′ end of said sequence of SEQ ID NO:55 from nucleotide 540 to nucleotide 769.

In other embodiments, the present invention provides a composition comprising a protein, wherein said protein comprises an amino acid sequence selected from the group consisting of:
(a) the amino acid sequence of SEQ ID NO:56;
(b) the amino acid sequence of SEQ ID NO:56 from amino acid 1 to amino acid 53;
(c) a fragment of the amino acid sequence of SEQ ID NO:56, the fragment comprising eight contiguous amino acids of SEQ ID NO:56; and
(d) the amino acid sequence encoded by the cDNA insert of clone AS86_{—}1 deposited with the ATCC under accession number 98076; the protein being substantially free from other mammalian proteins. Preferably such protein comprises the amino acid sequence of SEQ ID NO:56 or the amino acid sequence of SEQ ID NO:56 from amino acid 1 to amino acid 53. In further preferred embodiments, the present invention provides a protein comprising a fragment of the amino acid sequence of SEQ ID NO:56 having biological activity, the fragment preferably comprising eight (more preferably twenty, most preferably thirty) contiguous amino acids of SEQ ID NO:56, or a protein comprising a fragment of the amino acid sequence of SEQ ID NO:56 having biological activity, the fragment comprising the amino acid sequence from amino acid 35 to amino acid 44 of SEQ ID NO:56.

In one embodiment, the present invention provides a composition comprising an isolated polynucleotide selected from the group consisting of:
(a) a polynucleotide comprising the nucleotide sequence of SEQ ID NO:57;
(b) a polynucleotide comprising the nucleotide sequence of SEQ ID NO:57 from nucleotide 454 to nucleotide 1020;
(c) a polynucleotide comprising the nucleotide sequence of SEQ ID NO:57 from nucleotide 568 to nucleotide 789;
(d) a polynucleotide comprising the nucleotide sequence of the full-length protein coding sequence of clone AS162_{—}1 deposited with the ATCC under accession number 98076;
(e) a polynucleotide encoding the full-length protein encoded by the cDNA insert of clone AS162_{—}1 deposited with the ATCC under accession number 98076;
(f) a polynucleotide comprising the nucleotide sequence of a mature protein coding sequence of clone AS162_{—}1 deposited with the ATCC under accession number 98076;
(g) a polynucleotide encoding a mature protein encoded by the cDNA insert of clone AS162_{—}1 deposited with the ATCC under accession number 98076;
(h) a polynucleotide encoding a protein comprising the amino acid sequence of SEQ ID NO:58;
(i) a polynucleotide encoding a protein comprising a fragment of the amino acid sequence of SEQ ID NO:58 having biological activity, the fragment comprising eight contiguous amino acids of SEQ ID NO:58;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:57.

Preferably, such polynucleotide comprises the nucleotide sequence of SEQ ID NO:57 from nucleotide 454 to nucleotide 1020; the nucleotide sequence of SEQ ID NO:57 from nucleotide 568 to nucleotide 789; the nucleotide sequence of the full-length protein coding sequence of clone AS162_{—}1 deposited with the ATCC under accession number 98076; or the nucleotide sequence of a mature protein coding sequence of clone AS162_{—}1 deposited with the ATCC under accession number 98076. In other preferred embodiments, the polynucleotide encodes the full-length or a mature protein encoded by the cDNA insert of clone AS162_{—}1 deposited with the ATCC under accession number 98076. In yet other preferred embodiments, the present invention provides a polynucleotide encoding a protein comprising the amino acid sequence of SEQ ID NO:58 from amino acid 39 to amino acid 112. In further preferred embodiments, the present invention provides a polynucleotide encoding a protein comprising a fragment of the amino acid sequence of SEQ ID NO:58 having biological activity, the fragment preferably comprising eight (more preferably twenty, most preferably thirty) contiguous amino acids of SEQ ID NO:58, or a polynucleotide encoding a protein comprising a fragment of the amino acid sequence of SEQ ID NO:58 having biological activity, the fragment comprising the amino acid sequence from amino acid 89 to amino acid 98 of SEQ ID NO:58.

Other embodiments provide the gene corresponding to the cDNA sequence of SEQ ID NO:57.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:57, but excluding the poly(A) tail at the 3′ end of SEQ ID NO:57; and
      (ab) the nucleotide sequence of the cDNA insert of clone AS162_{—}1 deposited with the ATCC under accession number 98076;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:57, but excluding the poly(A) tail at the 3′ end of SEQ ID NO:57; and
      (bb) the nucleotide sequence of the cDNA insert of clone AS162_{—}1 deposited with the ATCC under accession number 98076;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:57, and extending contiguously from a nucleotide sequence corresponding to the 5′ end of SEQ ID NO:57 to a nucleotide sequence corresponding to the 3′ end of SEQ ID NO:57, but excluding the poly(A) tail at the 3′ end of SEQ ID NO:57. Also preferably the polynucleotide isolated according to the above process comprises a nucleotide sequence corresponding to the cDNA sequence of SEQ ID NO:57 from nucleotide 454 to nucleotide 1020, and extending contiguously from a nucleotide sequence corresponding to the 5′ end of said sequence of SEQ ID NO:57 from nucleotide 454 to nucleotide 1020, to a nucleotide sequence corresponding to the 3′ end of said sequence of SEQ ID NO:57 from nucleotide 454 to nucleotide 1020. Also preferably the polynucleotide isolated according to the above process comprises a nucleotide sequence corresponding to the cDNA sequence of SEQ ID NO:57 from nucleotide 568 to nucleotide 789, and extending contiguously from a nucleotide sequence corresponding to the 5′ end of said sequence of SEQ ID NO:57 from nucleotide 568 to nucleotide 789, to a nucleotide sequence corresponding to the 3′ end of said sequence of SEQ ID NO:57 from nucleotide 568 to nucleotide 789.

In other embodiments, the present invention provides a composition comprising a protein, wherein said protein comprises an amino acid sequence selected from the group consisting of:
(a) the amino acid sequence of SEQ ID NO:58;
(b) the amino acid sequence of SEQ ID NO:58 from amino acid 39 to amino acid 112;
(c) a fragment of the amino acid sequence of SEQ ID NO:58, the fragment comprising eight contiguous amino acids of SEQ ID NO:58; and
(d) the amino acid sequence encoded by the cDNA insert of clone AS162_{—}1 deposited with the ATCC under accession number 98076;
   the protein being substantially free from other mammalian proteins. Preferably such protein comprises the amino acid sequence of SEQ ID NO:58 or the amino acid sequence of SEQ ID NO:58 from amino acid 39 to amino acid 112. In further preferred embodiments, the present invention provides a protein comprising a fragment of the amino acid sequence of SEQ ID NO:58 having biological activity, the fragment preferably comprising eight (more preferably twenty, most preferably thirty) contiguous amino acids of SEQ ID NO:58, or a protein comprising a fragment of the amino acid sequence of SEQ ID NO:58 having biological activity, the fragment comprising the amino acid sequence from amino acid 89 to amino acid 98 of SEQ ID NO:58.

In one embodiment, the present invention provides a composition comprising an isolated polynucleotide selected from the group consisting of:
(a) a polynucleotide comprising the nucleotide sequence of SEQ ID NO:59;
(b) a polynucleotide comprising the nucleotide sequence of SEQ ID NO:59 from nucleotide 576 to nucleotide 1034;
(c) a polynucleotide comprising the nucleotide sequence of SEQ ID NO:59 from nucleotide 2189 to nucleotide 2263;
(d) a polynucleotide comprising the nucleotide sequence of the full-length protein coding sequence of clone AS264_{—}3 deposited with the ATCC under accession number 98076;
(e) a polynucleotide encoding the full-length protein encoded by the cDNA insert of clone AS264_{—}3 deposited with the ATCC under accession number 98076;
(f) a polynucleotide comprising the nucleotide sequence of a mature protein coding sequence of clone AS264_{—}3 deposited with the ATCC under accession number 98076;
(g) a polynucleotide encoding a mature protein encoded by the cDNA insert of clone AS264_{—}3 deposited with the ATCC under accession number 98076;
(h) a polynucleotide encoding a protein comprising the amino acid sequence of SEQ ID NO:60;
(i) a polynucleotide encoding a protein comprising a fragment of the amino acid sequence of SEQ ID NO:60 having biological activity, the fragment comprising eight contiguous amino acids of SEQ ID NO:60;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:59.

Preferably, such polynucleotide comprises the nucleotide sequence of SEQ ID NO:59 from nucleotide 576 to nucleotide 1034; the nucleotide sequence of SEQ ID NO:59 from nucleotide 2189 to nucleotide 2263; the nucleotide sequence of the full-length protein coding sequence of clone AS264_{—}3 deposited with the ATCC under accession number 98076; or the nucleotide sequence of a mature protein coding sequence of clone AS264_{—}3 deposited with the ATCC under accession number 98076. In other preferred embodiments, the polynucleotide encodes the full-length or a mature protein encoded by the cDNA insert of clone AS264_{—}3 deposited with the ATCC under accession number 98076. In further preferred embodiments, the present invention provides a polynucleotide encoding a protein comprising a fragment of the amino acid sequence of SEQ ID NO:60 having biological activity, the fragment preferably comprising eight (more preferably twenty, most preferably thirty) contiguous amino acids of SEQ ID NO:60, or a polynucleotide encoding a protein comprising a fragment of the amino acid sequence of SEQ ID NO:60 having biological activity, the fragment comprising the amino acid sequence from amino acid 71 to amino acid 80 of SEQ ID NO:60.

Other embodiments provide the gene corresponding to the cDNA sequence of SEQ ID NO:59.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:59, but excluding the poly(A) tail at the 3′ end of SEQ ID NO:59; and
      (ab) the nucleotide sequence of the cDNA insert of clone AS264_{—}3 deposited with the ATCC under accession number 98076;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:59, but excluding the poly(A) tail at the 3′ end of SEQ ID NO:59; and
      (bb) the nucleotide sequence of the cDNA insert of clone AS264_{—}3 deposited with the ATCC under accession number 98076;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:59, and extending contiguously from a nucleotide sequence corresponding to the 5′ end of SEQ ID NO:59 to a nucleotide sequence corresponding to the 3′ end of SEQ ID NO:59, but excluding the poly(A) tail at the 3′ end of SEQ ID NO:59. Also preferably the polynucleotide isolated according to the above process comprises a nucleotide sequence corresponding to the cDNA sequence of SEQ ID NO:59 from nucleotide 576 to nucleotide 1034, and extending contiguously from a nucleotide sequence corresponding to the 5′ end of said sequence of SEQ ID NO:59 from nucleotide 576 to nucleotide 1034, to a nucleotide sequence corresponding to the 3′ end of said sequence of SEQ ID NO:59 from nucleotide 576 to nucleotide 1034. Also preferably the polynucleotide isolated according to the above process comprises a nucleotide sequence corresponding to the cDNA sequence of SEQ ID NO:59 from nucleotide 2189 to nucleotide 2263, and extending contiguously from a nucleotide sequence corresponding to the 5′ end of said sequence of SEQ ID NO:59 from nucleotide 2189 to nucleotide 2263, to a nucleotide sequence corresponding to the 3′ end of said sequence of SEQ ID NO:59 from nucleotide 2189 to nucleotide 2263.

In other embodiments, the present invention provides a composition comprising a protein, wherein said protein comprises an amino acid sequence selected from the group consisting of:
(a) the amino acid sequence of SEQ ID NO:60;
(b) a fragment of the amino acid sequence of SEQ ID NO:60, the fragment comprising eight contiguous amino acids of SEQ ID NO:60; and
(c) the amino acid sequence encoded by the cDNA insert of clone AS264_{—}3 deposited with the ATCC under accession number 98076;
   the protein being substantially free from other mammalian proteins. Preferably such protein comprises the amino acid sequence of SEQ ID NO:60. In further preferred embodiments, the present invention provides a protein comprising a fragment of the amino acid sequence of SEQ ID NO:60 having biological activity, the fragment preferably comprising eight (more preferably twenty, most preferably thirty) contiguous amino acids of SEQ ID NO:60, or a protein comprising a fragment of the amino acid sequence of SEQ ID NO:60 having biological activity, the fragment comprising the amino acid sequence from amino acid 71 to amino acid 80 of SEQ ID NO:60.

In one embodiment, the present invention provides a composition comprising an isolated polynucleotide selected from the group consisting of:
(a) a polynucleotide comprising the nucleotide sequence of SEQ ID NO:61;
(b) a polynucleotide comprising the nucleotide sequence of SEQ ID NO:61 from nucleotide 164 to nucleotide 298;
(c) a polynucleotide comprising the nucleotide sequence of SEQ ID NO:61 from nucleotide 164 to nucleotide 259;
(d) a polynucleotide comprising the nucleotide sequence of the full-length protein coding sequence of clone AS268_{—}1 deposited with the ATCC under accession number 98076;
(e) a polynucleotide encoding the full-length protein encoded by the cDNA insert of clone AS268_{—}1 deposited with the ATCC under accession number 98076;
(f) a polynucleotide comprising the nucleotide sequence of a mature protein coding sequence of clone AS268_{—}1 deposited with the ATCC under accession number 98076;
(g) a polynucleotide encoding a mature protein encoded by the cDNA insert of clone AS268_{—}1 deposited with the ATCC under accession number 98076;
(h) a polynucleotide encoding a protein comprising the amino acid sequence of SEQ ID NO:62;
(i) a polynucleotide encoding a protein comprising a fragment of the amino acid sequence of SEQ ID NO:62 having biological activity, the fragment comprising eight contiguous amino acids of SEQ ID NO:62;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:61.

Preferably, such polynucleotide comprises the nucleotide sequence of SEQ ID NO:61 from nucleotide 164 to nucleotide 298; the nucleotide sequence of SEQ ID NO:61 from nucleotide 164 to nucleotide 259; the nucleotide sequence of the full-length protein coding sequence of clone AS268_{—}1 deposited with the ATCC under accession number 98076; or the nucleotide sequence of a mature protein coding sequence of clone AS268_{—}1 deposited with the ATCC under accession number 98076. In other preferred embodiments, the polynucleotide encodes the full-length or a mature protein encoded by the cDNA insert of clone AS268_{—}1 deposited with the ATCC under accession number 98076. In yet other preferred embodiments, the present invention provides a polynucleotide encoding a protein comprising the amino acid sequence of SEQ ID NO:62 from amino acid 1 to amino acid 32. In further preferred embodiments, the present invention provides a polynucleotide encoding a protein comprising a fragment of the amino acid sequence of SEQ ID NO:62 having biological activity, the fragment preferably comprising eight (more preferably twenty, most preferably thirty) contiguous amino acids of SEQ ID NO:62, or a polynucleotide encoding a protein comprising a fragment of the amino acid sequence of SEQ ID NO:62 having biological activity, the fragment comprising the amino acid sequence from amino acid 17 to amino acid 26 of SEQ ID NO:62.

Other embodiments provide the gene corresponding to the cDNA sequence of SEQ ID NO:61 and SEQ ID NO:63.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:61;
      (ab) SEQ ID NO:63, but excluding the poly(A) tail at the 3′ end of SEQ ID NO:63; and
      (ac) the nucleotide sequence of the cDNA insert of clone AS268_{—}1 deposited with the ATCC under accession number 98076;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:61;
      (bb) SEQ ID NO:63, but excluding the poly(A) tail at the 3′ end of SEQ ID NO:63; and
      (bc) the nucleotide sequence of the cDNA insert of clone AS268_{—}1 deposited with the ATCC under accession number 98076;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:61 and SEQ ID NO:63, and extending contiguously from a nucleotide sequence corresponding to the 5′ end of SEQ ID NO:61 to a nucleotide sequence corresponding to the 3′ end of SEQ ID NO:63, but excluding the poly(A) tail at the 3′ end of SEQ ID NO:63. Also preferably the polynucleotide isolated according to the above process comprises a nucleotide sequence corresponding to the cDNA sequence of SEQ ID NO:61, and extending contiguously from a nucleotide sequence corresponding to the 5′ end of SEQ ID NO:61 to a nucleotide sequence corresponding to the 3′ end of SEQ ID NO:61. Also preferably the polynucleotide isolated according to the above process comprises a nucleotide sequence corresponding to the cDNA sequence of SEQ ID NO:61 from nucleotide 164 to nucleotide 298, and extending contiguously from a nucleotide sequence corresponding to the 5′ end of said sequence of SEQ ID NO:61 from nucleotide 164 to nucleotide 298, to a nucleotide sequence corresponding to the 3′ end of said sequence of SEQ ID NO:61 from nucleotide 164 to nucleotide 298. Also preferably the polynucleotide isolated according to the above process comprises a nucleotide sequence corresponding to the cDNA sequence of SEQ ID NO:61 from nucleotide 164 to nucleotide 259, and extending contiguously from a nucleotide sequence corresponding to the 5′ end of said sequence of SEQ ID NO:61 from nucleotide 164 to nucleotide 259, to a nucleotide sequence corresponding to the 3′ end of said sequence of SEQ ID NO:61 from nucleotide 164 to nucleotide 259.

In other embodiments, the present invention provides a composition comprising a protein, wherein said protein comprises an amino acid sequence selected from the group consisting of:
(a) the amino acid sequence of SEQ ID NO:62;
(b) the amino acid sequence of SEQ ID NO:62 from amino acid 1 to amino acid 32;
(c) a fragment of the amino acid sequence of SEQ ID NO:62, the fragment comprising eight contiguous amino acids of SEQ ID NO:62; and
(d) the amino acid sequence encoded by the cDNA insert of clone AS268_{—}1 deposited with the ATCC under accession number 98076;
   the protein being substantially free from other mammalian proteins. Preferably such protein comprises the amino acid sequence of SEQ ID NO:62 or the amino acid sequence of SEQ ID NO:62 from amino acid 1 to amino acid 32. In further preferred embodiments, the present invention provides a protein comprising a fragment of the amino acid sequence of SEQ ID NO:62 having biological activity, the fragment preferably comprising eight (more preferably twenty, most preferably thirty) contiguous amino acids of SEQ ID NO:62, or a protein comprising a fragment of the amino acid sequence of SEQ ID NO:62 having biological activity, the fragment comprising the amino acid sequence from amino acid 17 to amino acid 26 of SEQ ID NO:62.

In one embodiment, the present invention provides a composition comprising an isolated polynucleotide selected from the group consisting of:
(a) a polynucleotide comprising the nucleotide sequence of SEQ ID NO:64;
(b) a polynucleotide comprising the nucleotide sequence of SEQ ID NO:64 from nucleotide 685 to nucleotide 1434;
(c) a polynucleotide comprising the nucleotide sequence of SEQ ID NO:64 from nucleotide 799 to nucleotide 1434;
(d) a polynucleotide comprising the nucleotide sequence of SEQ ID NO:64 from nucleotide 868 to nucleotide 1044;
(e) a polynucleotide comprising the nucleotide sequence of the full-length protein coding sequence of clone AS301_{—}2 deposited with the ATCC under accession number 98076;
(f) a polynucleotide encoding the full-length protein encoded by the cDNA insert of clone AS301_{—}2 deposited with the ATCC under accession number 98076;
(g) a polynucleotide comprising the nucleotide sequence of a mature protein coding sequence of clone AS301_{—}2 deposited with the ATCC under accession number 98076;
(h) a polynucleotide encoding a mature protein encoded by the cDNA insert of clone AS301_{—}2 deposited with the ATCC under accession number 98076;
(i) a polynucleotide encoding a protein comprising the amino acid sequence of SEQ ID NO:65;
(j) a polynucleotide encoding a protein comprising a fragment of the amino acid sequence of SEQ ID NO:65 having biological activity, the fragment comprising eight contiguous amino acids of SEQ ID NO:65;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:64.

Preferably, such polynucleotide comprises the nucleotide sequence of SEQ ID NO:64 from nucleotide 685 to nucleotide 1434; the nucleotide sequence of SEQ ID NO:64 from nucleotide 799 to nucleotide 1434; the nucleotide sequence of SEQ ID NO:64 from nucleotide 868 to nucleotide 1044; the nucleotide sequence of the full-length protein coding sequence of clone AS301_{—}2 deposited with the ATCC under accession number 98076; or the nucleotide sequence of a mature protein coding sequence of clone AS301_{—}2 deposited with the ATCC under accession number 98076. In other preferred embodiments, the polynucleotide encodes the full-length or a mature protein encoded by the cDNA insert of clone AS301_{—}2 deposited with the ATCC under accession number 98076. In yet other preferred embodiments, the present invention provides a polynucleotide encoding a protein comprising the amino acid sequence of SEQ ID NO:65 from amino acid 15 to amino acid 24. In further preferred embodiments, the present invention provides a polynucleotide encoding a protein comprising a fragment of the amino acid sequence of SEQ ID NO:65 having biological activity, the fragment preferably comprising eight (more preferably twenty, most preferably thirty) contiguous amino acids of SEQ ID NO:65, or a polynucleotide encoding a protein comprising a fragment of the amino acid sequence of SEQ ID NO:65 having biological activity, the fragment comprising the amino acid sequence from amino acid 120 to amino acid 129 of SEQ ID NO:65.

Other embodiments provide the gene corresponding to the cDNA sequence of SEQ ID NO:64.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:64; and
      (ab) the nucleotide sequence of the cDNA insert of clone AS301_{—}2 deposited with the ATCC under accession number 98076;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:64; and
      (bb) the nucleotide sequence of the cDNA insert of clone AS301_{—}2 deposited with the ATCC under accession number 98076;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:64, and extending contiguously from a nucleotide sequence corresponding to the 5′ end of SEQ ID NO:64 to a nucleotide sequence corresponding to the 3′ end of SEQ ID NO:64. Also preferably the polynucleotide isolated according to the above process comprises a nucleotide sequence corresponding to the cDNA sequence of SEQ ID NO:64 from nucleotide 685 to nucleotide 1434, and extending contiguously from a nucleotide sequence corresponding to the 5′ end of said sequence of SEQ ID NO:64 from nucleotide 685 to nucleotide 1434, to a nucleotide sequence corresponding to the 3′ end of said sequence of SEQ ID NO:64 from nucleotide 685 to nucleotide 1434. Also preferably the polynucleotide isolated according to the above process comprises a nucleotide sequence corresponding to the cDNA sequence of SEQ ID NO:64 from nucleotide 799 to nucleotide 1434, and extending contiguously from a nucleotide sequence corresponding to the 5′ end of said sequence of SEQ ID NO:64 from nucleotide 799 to nucleotide 1434, to a nucleotide sequence corresponding to the 3′ end of said sequence of SEQ ID NO:64 from nucleotide 799 to nucleotide 1434. Also preferably the polynucleotide isolated according to the above process comprises a nucleotide sequence corresponding to the cDNA sequence of SEQ ID NO:64 from nucleotide 868 to nucleotide 1044, and extending contiguously from a nucleotide sequence corresponding to the 5′ end of said sequence of SEQ ID NO:64 from nucleotide 868 to nucleotide 1044, to a nucleotide sequence corresponding to the 3′ end of said sequence of SEQ ID NO:64 from nucleotide 868 to nucleotide 1044.

In other embodiments, the present invention provides a composition comprising a protein, wherein said protein comprises an amino acid sequence selected from the group consisting of:
(a) the amino acid sequence of SEQ ID NO:65;
(b) the amino acid sequence of SEQ ID NO:65 from amino acid 15 to amino acid 24;
(c) a fragment of the amino acid sequence of SEQ ID NO:65, the fragment comprising eight contiguous amino acids of SEQ ID NO:65; and
(d) the amino acid sequence encoded by the cDNA insert of clone AS301_{—}2 deposited with the ATCC under accession number 98076;
   the protein being substantially free from other mammalian proteins. Preferably such protein comprises the amino acid sequence of SEQ ID NO:65 or the amino acid sequence of SEQ ID NO:65 from amino acid 15 to amino acid 24. In further preferred embodiments, the present invention provides a protein comprising a fragment of the amino acid sequence of SEQ ID NO:65 having biological activity, the fragment preferably comprising eight (more preferably twenty, most preferably thirty) contiguous amino acids of SEQ ID NO:65, or a protein comprising a fragment of the amino acid sequence of SEQ ID NO:65 having biological activity, the fragment comprising the amino acid sequence from amino acid 120 to amino acid 129 of SEQ ID NO:65.

In one embodiment, the present invention provides a composition comprising an isolated polynucleotide selected from the group consisting of:
(a) a polynucleotide comprising the nucleotide sequence of SEQ ID NO:66;
(b) a polynucleotide comprising the nucleotide sequence of SEQ ID NO:66 from nucleotide 288 to nucleotide 1799;
(c) a polynucleotide comprising the nucleotide sequence of SEQ ID NO:66 from nucleotide 441 to nucleotide 1799;
(d) a polynucleotide comprising the nucleotide sequence of SEQ ID NO:66 from nucleotide 250 to nucleotide 614;
(e) a polynucleotide comprising the nucleotide sequence of the full-length protein coding sequence of clone AU105_{—}14 deposited with the ATCC under accession number 98076;
(f) a polynucleotide encoding the full-length protein encoded by the cDNA insert of clone AU105_{—}14 deposited with the ATCC under accession number 98076;
(g) a polynucleotide comprising the nucleotide sequence of a mature protein coding sequence of clone AU105_{—}14 deposited with the ATCC under accession number 98076;
(h) a polynucleotide encoding a mature protein encoded by the cDNA insert of clone AU105_{—}14 deposited with the ATCC under accession number 98076;
(i) a polynucleotide encoding a protein comprising the amino acid sequence of SEQ ID NO:67;
(j) a polynucleotide encoding a protein comprising a fragment of the amino acid sequence of SEQ ID NO:67 having biological activity, the fragment comprising eight contiguous amino acids of SEQ ID NO:67;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:66.

Preferably, such polynucleotide comprises the nucleotide sequence of SEQ ID NO:66 from nucleotide 288 to nucleotide 1799; the nucleotide sequence of SEQ ID NO:66 from nucleotide 441 to nucleotide 1799; the nucleotide sequence of SEQ ID NO:66 from nucleotide 250 to nucleotide 614; the nucleotide sequence of the full-length protein coding sequence of clone AU105_{—}14 deposited with the ATCC under accession number 98076; or the nucleotide sequence of a mature protein coding sequence of clone AU105_{—}14 deposited with the ATCC under accession number 98076. In other preferred embodiments, the polynucleotide encodes the full-length or a mature protein encoded by the cDNA insert of clone AU105_{—}14 deposited with the ATCC under accession number 98076. In yet other preferred embodiments, the present invention provides a polynucleotide encoding a protein comprising the amino acid sequence of SEQ ID NO:67 from amino acid 1 to amino acid 109. In further preferred embodiments, the present invention provides a polynucleotide encoding a protein comprising a fragment of the amino acid sequence of SEQ ID NO:67 having biological activity, the fragment preferably comprising eight (more preferably twenty, most preferably thirty) contiguous amino acids of SEQ ID NO:67, or a polynucleotide encoding a protein comprising a fragment of the amino acid sequence of SEQ ID NO:67 having biological activity, the fragment comprising the amino acid sequence from amino acid 247 to amino acid 256 of SEQ ID NO:67.

Other embodiments provide the gene corresponding to the cDNA sequence of SEQ ID NO:66.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:66, but excluding the poly(A) tail at the 3′ end of SEQ ID NO:66; and
      (ab) the nucleotide sequence of the cDNA insert of clone AU105_{—}14 deposited with the ATCC under accession number 98076;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:66, but excluding the poly(A) tail at the 3′ end of SEQ ID NO:66; and
      (bb) the nucleotide sequence of the cDNA insert of clone AU105_{—}14 deposited with the ATCC under accession number 98076;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:66, and extending contiguously from a nucleotide sequence corresponding to the 5′ end of SEQ ID NO:66 to a nucleotide sequence corresponding to the 3′ end of SEQ ID NO:66, but excluding the poly(A) tail at the 3′ end of SEQ ID NO:66. Also preferably the polynucleotide isolated according to the above process comprises a nucleotide sequence corresponding to the cDNA sequence of SEQ ID NO:66 from nucleotide 288 to nucleotide 1799, and extending contiguously from a nucleotide sequence corresponding to the 5′ end of said sequence of SEQ ID NO:66 from nucleotide 288 to nucleotide 1799, to a nucleotide sequence corresponding to the 3′ end of said sequence of SEQ ID NO:66 from nucleotide 288 to nucleotide 1799. Also preferably the polynucleotide isolated according to the above process comprises a nucleotide sequence corresponding to the cDNA sequence of SEQ ID NO:66 from nucleotide 441 to nucleotide 1799, and extending contiguously from a nucleotide sequence corresponding to the 5′ end of said sequence of SEQ ID NO:66 from nucleotide 441 to nucleotide 1799, to a nucleotide sequence corresponding to the 3′ end of said sequence of SEQ ID NO:66 from nucleotide 441 to nucleotide 1799. Also preferably the polynucleotide isolated according to the above process comprises a nucleotide sequence corresponding to the cDNA sequence of SEQ ID NO:66 from nucleotide 250 to nucleotide 614, and extending contiguously from a nucleotide sequence corresponding to the 5′ end of said sequence of SEQ ID NO:66 from nucleotide 250 to nucleotide 614, to a nucleotide sequence corresponding to the 3′ end of said sequence of SEQ ID NO:66 from nucleotide 250 to nucleotide 614.

In other embodiments, the present invention provides a composition comprising a protein, wherein said protein comprises an amino acid sequence selected from the group consisting of:
(a) the amino acid sequence of SEQ ID NO:67;
(b) the amino acid sequence of SEQ ID NO:67 from amino acid 1 to amino acid 109;
(c) a fragment of the amino acid sequence of SEQ ID NO:67, the fragment comprising eight contiguous amino acids of SEQ ID NO:67; and
(d) the amino acid sequence encoded by the cDNA insert of clone AU105_{—}14 deposited with the ATCC under accession number 98076;
   the protein being substantially free from other mammalian proteins. Preferably such protein comprises the amino acid sequence of SEQ ID NO:67 or the amino acid sequence of SEQ ID NO:67 from amino acid 1 to amino acid 109. In further preferred embodiments, the present invention provides a protein comprising a fragment of the amino acid sequence of SEQ ID NO:67 having biological activity, the fragment preferably comprising eight (more preferably twenty, most preferably thirty) contiguous amino acids of SEQ ID NO:67, or a protein comprising a fragment of the amino acid sequence of SEQ ID NO:67 having biological activity, the fragment comprising the amino acid sequence from amino acid 247 to amino acid 256 of SEQ ID NO:67.

In one embodiment, the present invention provides a composition comprising an isolated polynucleotide selected from the group consisting of:
(a) a polynucleotide comprising the nucleotide sequence of SEQ ID NO:68;
(b) a polynucleotide comprising the nucleotide sequence of SEQ ID NO:68 from nucleotide 28 to nucleotide 1500;
(c) a polynucleotide comprising the nucleotide sequence of SEQ ID NO:68 from nucleotide 227 to nucleotide 517;
(d) a polynucleotide comprising the nucleotide sequence of the full-length protein coding sequence of clone AU139_{—}2 deposited with the ATCC under accession number 98076;
(e) a polynucleotide encoding the full-length protein encoded by the cDNA insert of clone AU139_{—}2 deposited with the ATCC under accession number 98076;
(f) a polynucleotide comprising the nucleotide sequence of a mature protein coding sequence of clone AU139_{—}2 deposited with the ATCC under accession number 98076;
(g) a polynucleotide encoding a mature protein encoded by the cDNA insert of clone AU139_{—}2 deposited with the ATCC under accession number 98076;
(h) a polynucleotide encoding a protein comprising the amino acid sequence of SEQ ID NO:69;
(i) a polynucleotide encoding a protein comprising a fragment of the amino acid sequence of SEQ ID NO:69 having biological activity, the fragment comprising eight contiguous amino acids of SEQ ID NO:69;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:68.

Preferably, such polynucleotide comprises the nucleotide sequence of SEQ ID NO:68 from nucleotide 28 to nucleotide 1500; the nucleotide sequence of SEQ ID NO:68 from nucleotide 227 to nucleotide 517; the nucleotide sequence of the full-length protein coding sequence of clone AU139_{—}2 deposited with the ATCC under accession number 98076; or the nucleotide sequence of a mature protein coding sequence of clone AU139_{—}2 deposited with the ATCC under accession number 98076. In other preferred embodiments, the polynucleotide encodes the full-length or a mature protein encoded by the cDNA insert of clone AU139_{—}2 deposited with the ATCC under accession number 98076. In yet other preferred embodiments, the present invention provides a polynucleotide encoding a protein comprising the amino acid sequence of SEQ ID NO:69 from amino acid 68 to amino acid 163. In further preferred embodiments, the present invention provides a polynucleotide encoding a protein comprising a fragment of the amino acid sequence of SEQ ID NO:69 having biological activity, the fragment preferably comprising eight (more preferably twenty, most preferably thirty) contiguous amino acids of SEQ ID NO:69, or a polynucleotide encoding a protein comprising a fragment of the amino acid sequence of SEQ ID NO:69 having biological activity, the fragment comprising the amino acid sequence from amino acid 240 to amino acid 249 of SEQ ID NO:69.

Other embodiments provide the gene corresponding to the cDNA sequence of SEQ ID NO:68.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO: 68, but excluding the poly(A) tail at the 3′ end of SEQ ID NO:68; and
      (ab) the nucleotide sequence of the cDNA insert of clone AU139_{—}2 deposited with the ATCC under accession number 98076;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:68, but excluding the poly(A) tail at the 3′ end of SEQ ID NO:68; and
      (bb) the nucleotide sequence of the cDNA insert of clone AU139_{—}2 deposited with the ATCC under accession number 98076;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:68, and extending contiguously from a nucleotide sequence corresponding to the 5′ end of SEQ ID NO:68 to a nucleotide sequence corresponding to the 3′ end of SEQ ID NO:68, but excluding the poly(A) tail at the 3′ end of SEQ ID NO:68. Also preferably the polynucleotide isolated according to the above process comprises a nucleotide sequence corresponding to the cDNA sequence of SEQ ID NO:68 from nucleotide 28 to nucleotide 1500, and extending contiguously from a nucleotide sequence corresponding to the 5′ end of said sequence of SEQ ID NO:68 from nucleotide 28 to nucleotide 1500, to a nucleotide sequence corresponding to the 3′ end of said sequence of SEQ ID NO:68 from nucleotide 28 to nucleotide 1500. Also preferably the polynucleotide isolated according to the above process comprises a nucleotide sequence corresponding to the cDNA sequence of SEQ ID NO:68 from nucleotide 227 to nucleotide 517, and extending contiguously from a nucleotide sequence corresponding to the 5′ end of said sequence of SEQ ID NO:68 from nucleotide 227 to nucleotide 517, to a nucleotide sequence corresponding to the 3′ end of said sequence of SEQ ID NO:68 from nucleotide 227 to nucleotide 517.

In other embodiments, the present invention provides a composition comprising a protein, wherein said protein comprises an amino acid sequence selected from the group consisting of:
(a) the amino acid sequence of SEQ ID NO:69;
(b) the amino acid sequence of SEQ ID NO:69 from amino acid 68 to amino acid 163;
(c) a fragment of the amino acid sequence of SEQ ID NO:69, the fragment comprising eight contiguous amino acids of SEQ ID NO:69; and
(d) the amino acid sequence encoded by the cDNA insert of clone AU139_{—}2 deposited with the ATCC under accession number 98076;
   the protein being substantially free from other mammalian proteins. Preferably such protein comprises the amino acid sequence of SEQ ID NO:69 or the amino acid sequence of SEQ ID NO:69 from amino acid 68 to amino acid 163. In further preferred embodiments, the present invention provides a protein comprising a fragment of the amino acid sequence of SEQ ID NO:69 having biological activity, the fragment preferably comprising eight (more preferably twenty, most preferably thirty) contiguous amino acids of SEQ ID NO:69, or a protein comprising a fragment of the amino acid sequence of SEQ ID NO:69 having biological activity, the fragment comprising the amino acid sequence from amino acid 240 to amino acid 249 of SEQ ID NO:69.

In one embodiment, the present invention provides a composition comprising an isolated polynucleotide selected from the group consisting of:
(a) a polynucleotide comprising the nucleotide sequence of SEQ ID NO:70;
(b) a polynucleotide comprising the nucleotide sequence of SEQ ID NO:70 from nucleotide 9 to nucleotide 1274;
(c) a polynucleotide comprising the nucleotide sequence of SEQ ID NO:70 from nucleotide 604 to nucleotide 745;
(d) a polynucleotide comprising the nucleotide sequence of the full-length protein coding sequence of clone AZ302_{—}1 deposited with the ATCC under accession number 98076;
(e) a polynucleotide encoding the full-length protein encoded by the cDNA insert of clone AZ302_{—}1 deposited with the ATCC under accession number 98076;
(f) a polynucleotide comprising the nucleotide sequence of a mature protein coding sequence of clone AZ302_{—}1 deposited with the ATCC under accession number 98076;
(g) a polynucleotide encoding a mature protein encoded by the cDNA insert of clone AZ302_{—}1 deposited with the ATCC under accession number 98076;
(h) a polynucleotide encoding a protein comprising the amino acid sequence of SEQ ID NO:71;
(i) a polynucleotide encoding a protein comprising a fragment of the amino acid sequence of SEQ ID NO:71 having biological activity, the fragment comprising eight contiguous amino acids of SEQ ID NO:71;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:70.

Preferably, such polynucleotide comprises the nucleotide sequence of SEQ ID NO:70 from nucleotide 9 to nucleotide 1274; the nucleotide sequence of SEQ ID NO:70 from nucleotide 604 to nucleotide 745; the nucleotide sequence of the full-length protein coding sequence of clone AZ302_{—}1 deposited with the ATCC under accession number 98076; or the nucleotide sequence of a mature protein coding sequence of clone AZ302_{—}1 deposited with the ATCC under accession number 98076. In other preferred embodiments, the polynucleotide encodes the full-length or a mature protein encoded by the cDNA insert of clone AZ302_{—}1 deposited with the ATCC under accession number 98076. In further preferred embodiments, the present invention provides a polynucleotide encoding a protein comprising a fragment of the amino acid sequence of SEQ ID NO:71 having biological activity, the fragment preferably comprising eight (more preferably twenty, most preferably thirty) contiguous amino acids of SEQ ID NO:71, or a polynucleotide encoding a protein comprising a fragment of the amino acid sequence of SEQ ID NO:71 having biological activity, the fragment comprising the amino acid sequence from amino acid 206 to amino acid 215 of SEQ ID NO:71.

Other embodiments provide the gene corresponding to the cDNA sequence of SEQ ID NO:70.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:70, but excluding the poly(A) tail at the 3′ end of SEQ ID NO:70; and
      (ab) the nucleotide sequence of the cDNA insert of clone AZ302_{—}1 deposited with the ATCC under accession number 98076;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:70, but excluding the poly(A) tail at the 3′ end of SEQ ID NO: 70; and
      (bb) the nucleotide sequence of the cDNA insert of clone AZ302_{—}1 deposited with the ATCC under accession number 98076;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:70, and extending contiguously from a nucleotide sequence corresponding to the 5′ end of SEQ ID NO:70 to a nucleotide sequence corresponding to the 3′ end of SEQ ID NO:70, but excluding the poly(A) tail at the 3′ end of SEQ ID NO:70. Also preferably the polynucleotide isolated according to the above process comprises a nucleotide sequence corresponding to the cDNA sequence of SEQ ID NO:70 from nucleotide 9 to nucleotide 1274, and extending contiguously from a nucleotide sequence corresponding to the 5′ end of said sequence of SEQ ID NO:70 from nucleotide 9 to nucleotide 1274, to a nucleotide sequence corresponding to the 3′ end of said sequence of SEQ ID NO:70 from nucleotide 9 to nucleotide 1274. Also preferably the polynucleotide isolated according to the above process comprises a nucleotide sequence corresponding to the cDNA sequence of SEQ ID NO:70 from nucleotide 604 to nucleotide 745, and extending contiguously from a nucleotide sequence corresponding to the 5′ end of said sequence of SEQ ID NO:70 from nucleotide 604 to nucleotide 745, to a nucleotide sequence corresponding to the 3′ end of said sequence of SEQ ID NO:70 from nucleotide 604 to nucleotide 745.

In other embodiments, the present invention provides a composition comprising a protein, wherein said protein comprises an amino acid sequence selected from the group consisting of:
(a) the amino acid sequence of SEQ ID NO:71;
(b) a fragment of the amino acid sequence of SEQ ID NO:71, the fragment comprising eight contiguous amino acids of SEQ ID NO:71; and
(c) the amino acid sequence encoded by the cDNA insert of clone AZ302_{—}1 deposited with the ATCC under accession number 98076; the protein being substantially free from other mammalian proteins. Preferably such protein comprises the amino acid sequence of SEQ ID NO:71. In further preferred embodiments, the present invention provides a protein comprising a fragment of the amino acid sequence of SEQ ID NO:71 having biological activity, the fragment preferably comprising eight (more preferably twenty, most preferably thirty) contiguous amino acids of SEQ ID NO:71, or a protein comprising a fragment of the amino acid sequence of SEQ ID NO:71 having biological activity, the fragment comprising the amino acid sequence from amino acid 206 to amino acid 215 of SEQ ID NO:71.

In one embodiment, the present invention provides a composition comprising an isolated polynucleotide selected from the group consisting of:
(a) a polynucleotide comprising the nucleotide sequence of SEQ ID NO:72;
(b) a polynucleotide comprising the nucleotide sequence of SEQ ID NO:72 from nucleotide 217 to nucleotide 1356;
(c) a polynucleotide comprising the nucleotide sequence of SEQ ID NO:72 from nucleotide 910 to nucleotide 1356;
(d) a polynucleotide comprising the nucleotide sequence of SEQ ID NO:72 from nucleotide 753 to nucleotide 1133;
(e) a polynucleotide comprising the nucleotide sequence of the full-length protein coding sequence of clone D147_{—}17 deposited with the ATCC under accession number 98076;
(f) a polynucleotide encoding the full-length protein encoded by the cDNA insert of clone D147_{—}17 deposited with the ATCC under accession number 98076;
(g) a polynucleotide comprising the nucleotide sequence of a mature protein coding sequence of clone D147_{—}17 deposited with the ATCC under accession number 98076;
(h) a polynucleotide encoding a mature protein encoded by the cDNA insert of clone D147_{—}17 deposited with the ATCC under accession number 98076;
(i) a polynucleotide encoding a protein comprising the amino acid sequence of SEQ ID NO:73;
(j) a polynucleotide encoding a protein comprising a fragment of the amino acid sequence of SEQ ID NO:73 having biological activity, the fragment comprising eight contiguous amino acids of SEQ ID NO:73;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:72.

Preferably, such polynucleotide comprises the nucleotide sequence of SEQ ID NO:72 from nucleotide 217 to nucleotide 1356; the nucleotide sequence of SEQ ID NO:72 from nucleotide 910 to nucleotide 1356; the nucleotide sequence of SEQ ID NO:72 from nucleotide 753 to nucleotide 1133; the nucleotide sequence of the full-length protein coding sequence of clone D147_{—}17 deposited with the ATCC under accession number 98076; or the nucleotide sequence of a mature protein coding sequence of clone D147_{—}17 deposited with the ATCC under accession number 98076. In other preferred embodiments, the polynucleotide encodes the full-length or a mature protein encoded by the cDNA insert of clone D147_{—}17 deposited with the ATCC under accession number 98076. In yet other preferred embodiments, the present invention provides a polynucleotide encoding a protein comprising the amino acid sequence of SEQ ID NO:73 from amino acid 180 to amino acid 306. In further preferred embodiments, the present invention provides a polynucleotide encoding a protein comprising a fragment of the amino acid sequence of SEQ ID NO:73 having biological activity, the fragment preferably comprising eight (more preferably twenty, most preferably thirty) contiguous amino acids of SEQ ID NO:73, or a polynucleotide encoding a protein comprising a fragment of the amino acid sequence of SEQ ID NO:73 having biological activity, the fragment comprising the amino acid sequence from amino acid 185 to amino acid 194 of SEQ ID NO:73.

Other embodiments provide the gene corresponding to the cDNA sequence of SEQ ID NO:72.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:72, but excluding the poly(A) tail at the 3′ end of SEQ ID NO:72; and
      (ab) the nucleotide sequence of the cDNA insert of clone D147_{—}17 deposited with the ATCC under accession number 98076;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:72, but excluding the poly(A) tail at the 3′ end of SEQ ID NO:72; and
      (bb) the nucleotide sequence of the cDNA insert of clone D147_{—}17 deposited with the ATCC under accession number 98076;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:72, and extending contiguously from a nucleotide sequence corresponding to the 5′ end of SEQ ID NO:72 to a nucleotide sequence corresponding to the 3′ end of SEQ ID NO:72, but excluding the poly(A) tail at the 3′ end of SEQ ID NO:72. Also preferably the polynucleotide isolated according to the above process comprises a nucleotide sequence corresponding to the cDNA sequence of SEQ ID NO:72 from nucleotide 217 to nucleotide 1356, and extending contiguously from a nucleotide sequence corresponding to the 5′ end of said sequence of SEQ ID NO:72 from nucleotide 217 to nucleotide 1356, to a nucleotide sequence corresponding to the 3′ end of said sequence of SEQ ID NO:72 from nucleotide 217 to nucleotide 1356. Also preferably the polynucleotide isolated according to the above process comprises a nucleotide sequence corresponding to the cDNA sequence of SEQ ID NO:72 from nucleotide 910 to nucleotide 1356, and extending contiguously from a nucleotide sequence corresponding to the 5′ end of said sequence of SEQ ID NO:72 from nucleotide 910 to nucleotide 1356, to a nucleotide sequence corresponding to the 3′ end of said sequence of SEQ ID NO:72 from nucleotide 910 to nucleotide 1356. Also preferably the polynucleotide isolated according to the above process comprises a nucleotide sequence corresponding to the cDNA sequence of SEQ ID NO:72 from nucleotide 753 to nucleotide 1133, and extending contiguously from a nucleotide sequence corresponding to the 5′ end of said sequence of SEQ ID NO:72 from nucleotide 753 to nucleotide 1133, to a nucleotide sequence corresponding to the 3′ end of said sequence of SEQ ID NO:72 from nucleotide 753 to nucleotide 1133.

In other embodiments, the present invention provides a composition comprising a protein, wherein said protein comprises an amino acid sequence selected from the group consisting of:
(a) the amino acid sequence of SEQ ID NO:73;
(b) the amino acid sequence of SEQ ID NO:73 from amino acid 180 to amino acid 306;
(c) a fragment of the amino acid sequence of SEQ ID NO:73, the fragment comprising eight contiguous amino acids of SEQ ID NO:73; and
(d) the amino acid sequence encoded by the cDNA insert of clone D147_{—}17 deposited with the ATCC under accession number 98076;
   the protein being substantially free from other mammalian proteins. Preferably such protein comprises the amino acid sequence of SEQ ID NO:73 or the amino acid sequence of SEQ ID NO:73 from amino acid 180 to amino acid 306. In further preferred embodiments, the present invention provides a protein comprising a fragment of the amino acid sequence of SEQ ID NO:73 having biological activity, the fragment preferably comprising eight (more preferably twenty, most preferably thirty) contiguous amino acids of SEQ ID NO:73, or a protein comprising a fragment of the amino acid sequence of SEQ ID NO:73 having biological activity, the fragment comprising the amino acid sequence from amino acid 185 to amino acid 194 of SEQ ID NO:73.

In one embodiment, the present invention provides a composition comprising an isolated polynucleotide selected from the group consisting of:
(a) a polynucleotide comprising the nucleotide sequence of SEQ ID NO:74;
(b) a polynucleotide comprising the nucleotide sequence of SEQ ID NO:74 from nucleotide 7 to nucleotide 264;
(c) a polynucleotide comprising the nucleotide sequence of SEQ ID NO:74 from nucleotide 55 to nucleotide 264;
(d) a polynucleotide comprising the nucleotide sequence of SEQ ID NO:74 from nucleotide 7 to nucleotide 180;
(e) a polynucleotide comprising the nucleotide sequence of the full-length protein coding sequence of clone O75_{—}9 deposited with the ATCC under accession number 98076;
(f) a polynucleotide encoding the full-length protein encoded by the cDNA insert of clone O75_{—}9 deposited with the ATCC under accession number 98076;
(g) a polynucleotide comprising the nucleotide sequence of a mature protein coding sequence of clone O75_{—}9 deposited with the ATCC under accession number 98076;
(h) a polynucleotide encoding a mature protein encoded by the cDNA insert of clone O75_{—}9 deposited with the ATCC under accession number 98076;
(i) a polynucleotide encoding a protein comprising the amino acid sequence of SEQ ID NO:75;
(j) a polynucleotide encoding a protein comprising a fragment of the amino acid sequence of SEQ ID NO:75 having biological activity, the fragment comprising eight contiguous amino acids of SEQ ID NO:75;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:74.

Preferably, such polynucleotide comprises the nucleotide sequence of SEQ ID NO:74 from nucleotide 7 to nucleotide 264; the nucleotide sequence of SEQ ID NO:74 from nucleotide 55 to nucleotide 264; the nucleotide sequence of SEQ ID NO:74 from nucleotide 7 to nucleotide 180; the nucleotide sequence of the full-length protein coding sequence of clone O75_{—}9 deposited with the ATCC under accession number 98076; or the nucleotide sequence of a mature protein coding sequence of clone O75_{—}9 deposited with the ATCC under accession number 98076. In other preferred embodiments, the polynucleotide encodes the full-length or a mature protein encoded by the cDNA insert of clone O75_{—}9 deposited with the ATCC under accession number 98076. In yet other preferred embodiments, the present invention provides a polynucleotide encoding a protein comprising the amino acid sequence of SEQ ID NO:75 from amino acid 1 to amino acid 58. In further preferred embodiments, the present invention provides a polynucleotide encoding a protein comprising a fragment of the amino acid sequence of SEQ ID NO:75 having biological activity, the fragment preferably comprising eight (more preferably twenty, most preferably thirty) contiguous amino acids of SEQ ID NO:75, or a polynucleotide encoding a protein comprising a fragment of the amino acid sequence of SEQ ID NO:75 having biological activity, the fragment comprising the amino acid sequence from amino acid 38 to amino acid 47 of SEQ ID NO:75.

Other embodiments provide the gene corresponding to the cDNA sequence of SEQ ID NO:74 and SEQ ID NO:76.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:74;
      (ab) SEQ ID NO:76; and
      (ac) the nucleotide sequence of the cDNA insert of clone O75_{—}9 deposited with the ATCC under accession number 98076;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:74;
      (bb) SEQ ID NO:76; and
      (bc) the nucleotide sequence of the cDNA insert of clone O75_{—}9 deposited with the ATCC under accession number 98076;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:74 and SEQ ID NO:76, and extending contiguously from a nucleotide sequence corresponding to the 5′ end of SEQ ID NO:74 to a nucleotide sequence corresponding to the 3′ end of SEQ ID NO:76. Also preferably the polynucleotide isolated according to the above process comprises a nucleotide sequence corresponding to the cDNA sequence of SEQ ID NO:74, and extending contiguously from a nucleotide sequence corresponding to the 5′ end of SEQ ID NO:74 to a nucleotide sequence corresponding to the 3′ end of SEQ ID NO:74. Also preferably the polynucleotide isolated according to the above process comprises a nucleotide sequence corresponding to the cDNA sequence of SEQ ID NO:74 from nucleotide 7 to nucleotide 264, and extending contiguously from a nucleotide sequence corresponding to the 5′ end of said sequence of SEQ ID NO:74 from nucleotide 7 to nucleotide 264, to a nucleotide sequence corresponding to the 3′ end of said sequence of SEQ ID NO:74 from nucleotide 7 to nucleotide 264. Also preferably the polynucleotide isolated according to the above process comprises a nucleotide sequence corresponding to the cDNA sequence of SEQ ID NO:74 from nucleotide 55 to nucleotide 264, and extending contiguously from a nucleotide sequence corresponding to the 5′ end of said sequence of SEQ ID NO:74 from nucleotide 55 to nucleotide 264, to a nucleotide sequence corresponding to the 3′ end of said sequence of SEQ ID NO:74 from nucleotide 55 to nucleotide 264. Also preferably the polynucleotide isolated according to the above process comprises a nucleotide sequence corresponding to the cDNA sequence of SEQ ID NO:74 from nucleotide 7 to nucleotide 180, and extending contiguously from a nucleotide sequence corresponding to the 5′ end of said sequence of SEQ ID NO:74 from nucleotide 7 to nucleotide 180, to a nucleotide sequence corresponding to the 3′ end of said sequence of SEQ ID NO:74 from nucleotide 7 to nucleotide 180.

In other embodiments, the present invention provides a composition comprising a protein, wherein said protein comprises an amino acid sequence selected from the group consisting of:
(a) the amino acid sequence of SEQ ID NO:75;
(b) the amino acid sequence of SEQ ID NO:75 from amino acid 1 to amino acid 58;
(c) a fragment of the amino add sequence of SEQ ID NO:75, the fragment comprising eight contiguous amino adds of SEQ ID NO:75; and
(d) the amino acid sequence encoded by the cDNA insert of clone O75_{—}9 deposited with the ATCC under accession number 98076;
   the protein being substantially free from other mammalian proteins. Preferably such protein comprises the amino acid sequence of SEQ ID NO:75 or the amino acid sequence of SEQ ID NO:75 from amino acid 1 to amino acid 58. In further preferred embodiments, the present invention provides a protein comprising a fragment of the amino acid sequence of SEQ ID NO:75 having biological activity, the fragment preferably comprising eight (more preferably twenty, most preferably thirty) contiguous amino adds of SEQ ID NO:75, or a protein comprising a fragment of the amino add sequence of SEQ ID NO:75 having biological activity, the fragment comprising the amino acid sequence from amino acid 38 to amino acid 47 of SEQ ID NO:75.

In one embodiment, the present invention provides a composition comprising an isolated polynucleotide selected from the group consisting of:
(a) a polynucleotide comprising the nucleotide sequence of SEQ ID NO:77;
(b) a polynucleotide comprising the nucleotide sequence of SEQ ID NO:77 from nucleotide 259 to nucleotide 480;
(c) a polynucleotide comprising the nucleotide sequence of SEQ ID NO:77 from nucleotide 325 to nucleotide 480;
(d) a polynucleotide comprising the nucleotide sequence of SEQ ID NO:77 from nucleotide 800 to nucleotide 892;
(e) a polynucleotide comprising the nucleotide sequence of the full-length protein coding sequence of clone AS152_{—}1 deposited with the ATCC under accession number 98079;
(f) a polynucleotide encoding the full-length protein encoded by the cDNA insert of clone AS152_{—}1 deposited with the ATCC under accession number 98079;
(g) a polynucleotide comprising the nucleotide sequence of a mature protein coding sequence of clone AS152_{—}1 deposited with the ATCC under accession number 98079;
(h) a polynucleotide encoding a mature protein encoded by the cDNA insert of clone AS152_{—}1 deposited with the ATCC under accession number 98079;
(i) a polynucleotide encoding a protein comprising the amino acid sequence of SEQ ID NO:78;
(j) a polynucleotide encoding a protein comprising a fragment of the amino acid sequence of SEQ ID NO:78 having biological activity, the fragment comprising eight contiguous amino acids of SEQ ID NO:78;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:77.

Preferably, such polynucleotide comprises the nucleotide sequence of SEQ ID NO:77 from nucleotide 259 to nucleotide 480; the nucleotide sequence of SEQ ID NO:77 from nucleotide 325 to nucleotide 480; the nucleotide sequence of SEQ ID NO:77 from nucleotide 800 to nucleotide 892; the nucleotide sequence of the full-length protein coding sequence of clone AS152_{—}1 deposited with the ATCC under accession number 98079; or the nucleotide sequence of a mature protein coding sequence of clone AS152_{—}1 deposited with the ATCC under accession number 98079. In other preferred embodiments, the polynucleotide encodes the full-length or a mature protein encoded by the cDNA insert of clone AS152_{—}1 deposited with the ATCC under accession number 98079. In further preferred embodiments, the present invention provides a polynucleotide encoding a protein comprising a fragment of the amino acid sequence of SEQ ID NO:78 having biological activity, the fragment preferably comprising eight (more preferably twenty, most preferably thirty) contiguous amino acids of SEQ ID NO:78, or a polynucleotide encoding a protein comprising a fragment of the amino acid sequence of SEQ ID NO:78 having biological activity, the fragment comprising the amino acid sequence from amino acid 32 to amino acid 41 of SEQ ID NO:78.

Other embodiments provide the gene corresponding to the cDNA sequence of SEQ ID NO:77 and SEQ ID NO:79.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:77;
      (ab) SEQ ID NO:79, but excluding the poly(A) tail at the 3′ end of SEQ ID NO:79; and
      (ac) the nucleotide sequence of the cDNA insert of clone AS152_{—}1 deposited with the ATCC under accession number 98079;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:77;
      (bb) SEQ ID NO:79, but excluding the poly(A) tail at the 3′ end of SEQ ID NO:79; and
      (bc) the nucleotide sequence of the cDNA insert of clone AS152_{—}1 deposited with the ATCC under accession number 98079;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:77 and SEQ ID NO:79, and extending contiguously from a nucleotide sequence corresponding to the 5′ end of SEQ ID NO:77 to a nucleotide sequence corresponding to the 3′ end of SEQ ID NO:79, but excluding the poly(A) tail at the 3′ end of SEQ ID NO:79. Also preferably the polynucleotide isolated according to the above process comprises a nucleotide sequence corresponding to the cDNA sequence of SEQ ID NO:77, and extending contiguously from a nucleotide sequence corresponding to the 5′ end of SEQ ID NO:77 to a nucleotide sequence corresponding to the 3′ end of SEQ ID NO:77. Also preferably the polynucleotide isolated according to the above process comprises a nucleotide sequence corresponding to the cDNA sequence of SEQ ID NO:77 from nucleotide 259 to nucleotide 480, and extending contiguously from a nucleotide sequence corresponding to the 5′ end of said sequence of SEQ ID NO:77 from nucleotide 259 to nucleotide 480, to a nucleotide sequence corresponding to the 3′ end of said sequence of SEQ ID NO:77 from nucleotide 259 to nucleotide 480. Also preferably the polynucleotide isolated according to the above process comprises a nucleotide sequence corresponding to the cDNA sequence of SEQ ID NO:77 from nucleotide 325 to nucleotide 480, and extending contiguously from a nucleotide sequence corresponding to the 5′ end of said sequence of SEQ ID NO:77 from nucleotide 325 to nucleotide 480, to a nucleotide sequence corresponding to the 3′ end of said sequence of SEQ ID NO:77 from nucleotide 325 to nucleotide 480. Also preferably the polynucleotide isolated according to the above process comprises a nucleotide sequence corresponding to the cDNA sequence of SEQ ID NO:77 from nucleotide 800 to nucleotide 892, and extending contiguously from a nucleotide sequence corresponding to the 5′ end of said sequence of SEQ ID NO:77 from nucleotide 800 to nucleotide 892, to a nucleotide sequence corresponding to the 3′ end of said sequence of SEQ ID NO:77 from nucleotide 800 to nucleotide 892.

In other embodiments, the present invention provides a composition comprising a protein, wherein said protein comprises an amino acid sequence selected from the group consisting of:
(a) the amino acid sequence of SEQ ID NO:78;
(b) a fragment of the amino acid sequence of SEQ ID NO:78, the fragment comprising eight contiguous amino acids of SEQ ID NO:78; and
(c) the amino acid sequence encoded by the cDNA insert of clone

AS152_{—}1 deposited with the ATCC under accession number 98079; the protein being substantially free from other mammalian proteins. Preferably such protein comprises the amino acid sequence of SEQ ID NO:78. In further preferred embodiments, the present invention provides a protein comprising a fragment of the amino acid sequence of SEQ ID NO:78 having biological activity, the fragment preferably comprising eight (more preferably twenty, most preferably thirty) contiguous amino acids of SEQ ID NO:78, or a protein comprising a fragment of the amino acid sequence of SEQ ID NO:78 having biological activity, the fragment comprising the amino acid sequence from amino acid 32 to amino acid 41 of SEQ ID NO:78.

In one embodiment, the present invention provides a composition comprising an isolated polynucleotide selected from the group consisting of:
(a) a polynucleotide comprising the nucleotide sequence of SEQ ID NO:80;
(b) a polynucleotide comprising the nucleotide sequence of SEQ ID NO:80 from nucleotide 84 to nucleotide 305;
(c) a polynucleotide comprising the nucleotide sequence of SEQ ID NO:80 from nucleotide 150 to nucleotide 305;
(d) a polynucleotide comprising the nucleotide sequence of SEQ ID NO:80 from nucleotide 459 to nucleotide 521;
(e) a polynucleotide comprising the nucleotide sequence of the full-length protein coding sequence of clone AS152_{—}2 deposited with the ATCC under accession number 98181;
(f) a polynucleotide encoding the full-length protein encoded by the cDNA insert of clone AS152_{—}2 deposited with the ATCC under accession number 98181;
(g) a polynucleotide comprising the nucleotide sequence of a mature protein coding sequence of clone AS152_{—}2 deposited with the ATCC under accession number 98181;
(h) a polynucleotide encoding a mature protein encoded by the cDNA insert of clone AS152_{—}2 deposited with the ATCC under accession number 98181;
(i) a polynucleotide encoding a protein comprising the amino acid sequence of SEQ ID NO:81;
(j) a polynucleotide encoding a protein comprising a fragment of the amino acid sequence of SEQ ID NO:81 having biological activity, the fragment comprising eight contiguous amino acids of SEQ ID NO:81;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:80.

Preferably, such polynucleotide comprises the nucleotide sequence of SEQ ID NO:80 from nucleotide 84 to nucleotide 305; the nucleotide sequence of SEQ ID NO:80 from nucleotide 150 to nucleotide 305; the nucleotide sequence of SEQ ID NO:80 from nucleotide 459 to nucleotide 521; the nucleotide sequence of the full-length protein coding sequence of clone AS152_{—}2 deposited with the ATCC under accession number 98181; or the nucleotide sequence of a mature protein coding sequence of clone AS152_{—}2 deposited with the ATCC under accession number 98181. In other preferred embodiments, the polynucleotide encodes the full-length or a mature protein encoded by the cDNA insert of clone AS152_{—}2 deposited with the ATCC under accession number 98181. In further preferred embodiments, the present invention provides a polynucleotide encoding a protein comprising a fragment of the amino acid sequence of SEQ ID NO:81 having biological activity, the fragment preferably comprising eight (more preferably twenty, most preferably thirty) contiguous amino acids of SEQ ID NO:81, or a polynucleotide encoding a protein comprising a fragment of the amino acid sequence of SEQ ID NO:81 having biological activity, the fragment comprising the amino acid sequence from amino acid 32 to amino acid 41 of SEQ ID NO:81.

Other embodiments provide the gene corresponding to the cDNA sequence of SEQ ID NO:80 and SEQ ID NO:82.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:80;
      (ab) SEQ ID NO:82, but excluding the poly(A) tail at the 3′ end of SEQ ID NO:82; and
      (ac) the nucleotide sequence of the cDNA insert of clone AS152_{—}2 deposited with the ATCC under accession number 98181;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:80;
      (bb) SEQ ID NO:82, but excluding the poly(A) tail at the 3′ end of SEQ ID NO:82; and
      (bc) the nucleotide sequence of the cDNA insert of clone AS152_{—}2 deposited with the ATCC under accession number 98181;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:80 and SEQ ID NO:82, and extending contiguously from a nucleotide sequence corresponding to the 5′ end of SEQ ID NO:80 to a nucleotide sequence corresponding to the 3′ end of SEQ ID NO:82, but excluding the poly(A) tail at the 3′ end of SEQ ID NO:82. Also preferably the polynucleotide isolated according to the above process comprises a nucleotide sequence corresponding to the cDNA sequence of SEQ ID NO:80, and extending contiguously from a nucleotide sequence corresponding to the 5′ end of SEQ ID NO:80 to a nucleotide sequence corresponding to the 3′ end of SEQ ID NO:80. Also preferably the polynucleotide isolated according to the above process comprises a nucleotide sequence corresponding to the cDNA sequence of SEQ ID NO:80 from nucleotide 84 to nucleotide 305, and extending contiguously from a nucleotide sequence corresponding to the 5′ end of said sequence of SEQ ID NO:80 from nucleotide 84 to nucleotide 305, to a nucleotide sequence corresponding to the 3′ end of said sequence of SEQ ID NO:80 from nucleotide 84 to nucleotide 305. Also preferably the polynucleotide isolated according to the above process comprises a nucleotide sequence corresponding to the cDNA sequence of SEQ ID NO:80 from nucleotide 150 to nucleotide 305, and extending contiguously from a nucleotide sequence corresponding to the 5′ end of said sequence of SEQ ID NO:80 from nucleotide 150 to nucleotide 305, to a nucleotide sequence corresponding to the 3′ end of said sequence of SEQ ID NO:80 from nucleotide 150 to nucleotide 305. Also preferably the polynucleotide isolated according to the above process comprises a nucleotide sequence corresponding to the cDNA sequence of SEQ ID NO:80 from nucleotide 459 to nucleotide 521, and extending contiguously from a nucleotide sequence corresponding to the 5′ end of said sequence of SEQ ID NO:80 from nucleotide 459 to nucleotide 521, to a nucleotide sequence corresponding to the 3′ end of said sequence of SEQ ID NO:80 from nucleotide 459 to nucleotide 521.

In other embodiments, the present invention provides a composition comprising a protein, wherein said protein comprises an amino acid sequence selected from the group consisting of:
(a) the amino acid sequence of SEQ ID NO:81;
(b) a fragment of the amino acid sequence of SEQ ID NO:81, the fragment comprising eight contiguous amino acids of SEQ [D NO:81; and
(c) the amino acid sequence encoded by the cDNA insert of clone AS152_{—}2 deposited with the ATCC under accession number 98181;
   the protein being substantially free from other mammalian proteins. Preferably such protein comprises the amino acid sequence of SEQ ID NO:81. In further preferred embodiments, the present invention provides a protein comprising a fragment of the amino acid sequence of SEQ ID NO:81 having biological activity, the fragment preferably comprising eight (more preferably twenty, most preferably thirty) contiguous amino acids of SEQ ID NO:81, or a protein comprising a fragment of the amino acid sequence of SEQ ID NO:81 having biological activity, the fragment comprising the amino acid sequence from amino acid 32 to amino acid 41 of SEQ ID NO:81.

In one embodiment, the present invention provides a composition comprising an isolated polynucleotide selected from the group consisting of:
(a) a polynucleotide comprising the nucleotide sequence of SEQ ID NO:83;
(b) a polynucleotide comprising the nucleotide sequence of SEQ ID NO:83 from nucleotide 510 to nucleotide 2189;
(c) a polynucleotide comprising the nucleotide sequence of SEQ ID NO:83 from nucleotide 570 to nucleotide 2189;
(d) a polynucleotide comprising the nucleotide sequence of SEQ ID NO:83 from nucleotide 1826 to nucleotide 2108;
(e) a polynucleotide comprising the nucleotide sequence of the full-length protein coding sequence of clone AS167_{—}3 deposited with the ATCC under accession number 98079;
(f) a polynucleotide encoding the full-length protein encoded by the cDNA insert of clone AS167_{—}3 deposited with the ATCC under accession number 98079;
(g) a polynucleotide comprising the nucleotide sequence of a mature protein coding sequence of clone AS167_{—}3 deposited with the ATCC under accession number 98079;
(h) a polynucleotide encoding a mature protein encoded by the cDNA insert of clone AS167_{—}3 deposited with the ATCC under accession number 98079;
(i) a polynucleotide encoding a protein comprising the amino acid sequence of SEQ ID NO:84;
(j) a polynucleotide encoding a protein comprising a fragment of the amino acid sequence of SEQ ID NO:84 having biological activity, the fragment comprising eight contiguous amino acids of SEQ ID NO:84;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:83.

Preferably, such polynucleotide comprises the nucleotide sequence of SEQ ID NO:83 from nucleotide 510 to nucleotide 2189; the nucleotide sequence of SEQ ID NO:83 from nucleotide 570 to nucleotide 2189; the nucleotide sequence of SEQ ID NO:83 from nucleotide 1826 to nucleotide 2108; the nucleotide sequence of the full-length protein coding sequence of clone AS167_{—}3 deposited with the ATCC under accession number 98079; or the nucleotide sequence of a mature protein coding sequence of clone AS167_{—}3 deposited with the ATCC under accession number 98079. In other preferred embodiments, the polynucleotide encodes the full-length or a mature protein encoded by the cDNA insert of clone AS167_{—}3 deposited with the ATCC under accession number 98079. In yet other preferred embodiments, the present invention provides a polynucleotide encoding a protein comprising the amino acid sequence of SEQ ID NO:84 from amino acid 440 to amino acid 533. In further preferred embodiments, the present invention provides a polynucleotide encoding a protein comprising a fragment of the amino acid sequence of SEQ ID NO:84 having biological activity, the fragment preferably comprising eight (more preferably twenty, most preferably thirty) contiguous amino acids of SEQ ID NO:84, or a polynucleotide encoding a protein comprising a fragment of the amino acid sequence of SEQ ID NO:84 having biological activity, the fragment comprising the amino acid sequence from amino acid 275 to amino acid 284 of SEQ ID NO:84.

Other embodiments provide the gene corresponding to the cDNA sequence of SEQ ID NO:83.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:83, but excluding the poly(A) tail at the 3′ end of SEQ ID NO:83; and
      (ab) the nucleotide sequence of the cDNA insert of clone AS167_{—}3 deposited with the ATCC under accession number 98079;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:83, but excluding the poly(A) tail at the 3′ end of SEQ ID NO:83; and
      (bb) the nucleotide sequence of the cDNA insert of clone AS167_{—}3 deposited with the ATCC under accession number 98079;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:83, and extending contiguously from a nucleotide sequence corresponding to the 5′ end of SEQ ID NO:83 to a nucleotide sequence corresponding to the 3′ end of SEQ ID NO:83, but excluding the poly(A) tail at the 3′ end of SEQ ID NO:83. Also preferably the polynucleotide isolated according to the above process comprises a nucleotide sequence corresponding to the cDNA sequence of SEQ ID NO:83 from nucleotide 510 to nucleotide 2189, and extending contiguously from a nucleotide sequence corresponding to the 5′ end of said sequence of SEQ ID NO:83 from nucleotide 510 to nucleotide 2189, to a nucleotide sequence corresponding to the 3 end of said sequence of SEQ ID NO:83 from nucleotide 510 to nucleotide 2189. Also preferably the polynucleotide isolated according to the above process comprises a nucleotide sequence corresponding to the cDNA sequence of SEQ ID NO:83 from nucleotide 570 to nucleotide 2189, and extending contiguously from a nucleotide sequence corresponding to the 5′ end of said sequence of SEQ ID NO:83 from nucleotide 570 to nucleotide 2189, to a nucleotide sequence corresponding to the 3′ end of said sequence of SEQ ID NO:83 from nucleotide 570 to nucleotide 2189. Also preferably the polynucleotide isolated according to the above process comprises a nucleotide sequence corresponding to the cDNA sequence of SEQ ID NO:83 from nucleotide 1826 to nucleotide 2108, and extending contiguously from a nucleotide sequence corresponding to the 5′ end of said sequence of SEQ ID NO:83 from nucleotide 1826 to nucleotide 2108, to a nucleotide sequence corresponding to the 3′ end of said sequence of SEQ ID NO:83 from nucleotide 1826 to nucleotide 2108.

In other embodiments, the present invention provides a composition comprising a protein, wherein said protein comprises an amino acid sequence selected from the group consisting of:
(a) the amino acid sequence of SEQ ID NO:84;
(b) the amino acid sequence of SEQ ID NO:84 from amino acid 440 to amino acid 533;
(c) a fragment of the amino acid sequence of SEQ ID NO:84, the fragment comprising eight contiguous amino acids of SEQ ID NO:84; and
(d) the amino acid sequence encoded by the cDNA insert of clone AS167_{—}3 deposited with the ATCC under accession number 98079;
   the protein being substantially free from other mammalian proteins. Preferably such protein comprises the amino acid sequence of SEQ ID NO:84 or the amino acid sequence of SEQ ID NO:84 from amino acid 440 to amino acid 533. In further preferred embodiments, the present invention provides a protein comprising a fragment of the amino acid sequence of SEQ ID NO:84 having biological activity, the fragment preferably comprising eight (more preferably twenty, most preferably thirty) contiguous amino acids of SEQ ID NO:84, or a protein comprising a fragment of the amino acid sequence of SEQ ID NO:84 having biological activity, the fragment comprising the amino acid sequence from amino acid 275 to amino acid 284 of SEQ ID NO:84.

In one embodiment, the present invention provides a composition comprising an isolated polynucleotide selected from the group consisting of:
(a) a polynucleotide comprising the nucleotide sequence of SEQ ID NO:85;
(b) a polynucleotide comprising the nucleotide sequence of SEQ ID NO:85 from nucleotide 19 to nucleotide 2433;
(c) a polynucleotide comprising the nucleotide sequence of SEQ ID NO:85 from nucleotide 70 to nucleotide 2433;
(d) a polynucleotide comprising the nucleotide sequence of SEQ ID NO:85 from nucleotide 156 to nucleotide 303;
(e) a polynucleotide comprising the nucleotide sequence of the full-length protein coding sequence of clone AU47_{—}8 deposited with the ATCC under accession number 98079;
(f) a polynucleotide encoding the full-length protein encoded by the cDNA insert of clone AU47_{—}8 deposited with the ATCC under accession number 98079;
(g) a polynucleotide comprising the nucleotide sequence of a mature protein coding sequence of clone AU47_{—}8 deposited with the ATCC under accession number 98079;
(h) a polynucleotide encoding a mature protein encoded by the cDNA insert of clone AU47_{—}8 deposited with the ATCC under accession number 98079;
(i) a polynucleotide encoding a protein comprising the amino acid sequence of SEQ ID NO:86;
(j) a polynucleotide encoding a protein comprising a fragment of the amino acid sequence of SEQ ID NO:86 having biological activity, the fragment comprising eight contiguous amino acids of SEQ ID NO:86;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:85.

Preferably, such polynucleotide comprises the nucleotide sequence of SEQ ID NO:85 from nucleotide 19 to nucleotide 2433; the nucleotide sequence of SEQ ID NO:85 from nucleotide 70 to nucleotide 2433; the nucleotide sequence of SEQ ID NO:85 from nucleotide 156 to nucleotide 303; the nucleotide sequence of the full-length protein coding sequence of clone AU47_{—}8 deposited with the ATCC under accession number 98079; or the nucleotide sequence of a mature protein coding sequence of clone AU47_{—}8 deposited with the ATCC under accession number 98079. In other preferred embodiments, the polynucleotide encodes the full-length or a mature protein encoded by the cDNA insert of clone AU47_{—}8 deposited with the ATCC under accession number 98079. In yet other preferred embodiments, the present invention provides a polynucleotide encoding a protein comprising the amino acid sequence of SEQ ID NO:86 from amino acid 47 to amino acid 56. In further preferred embodiments, the present invention provides a polynucleotide encoding a protein comprising a fragment of the amino acid sequence of SEQ ID NO:86 having biological activity, the fragment preferably comprising eight (more preferably twenty, most preferably thirty) contiguous amino acids of SEQ ID NO:86, or a polynucleotide encoding a protein comprising a fragment of the amino acid sequence of SEQ ID NO:86 having biological activity, the fragment comprising the amino acid sequence from amino acid 397 to amino acid 406 of SEQ ID NO:86.

Other embodiments provide the gene corresponding to the cDNA sequence of SEQ ID NO:85.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:85, but excluding the poly(A) tail at the 3′ end of SEQ ID NO:85; and
      (ab) the nucleotide sequence of the cDNA insert of clone AU47_{—}8 deposited with the ATCC under accession number 98079;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:85, but excluding the poly(A) tail at the 3′ end of SEQ ID NO:85; and
      (bb) the nucleotide sequence of the cDNA insert of clone AU47_{—}8 deposited with the ATCC under accession number 98079;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:85, and extending contiguously from a nucleotide sequence corresponding to the 5′ end of SEQ ID NO:85 to a nucleotide sequence corresponding to the 3′ end of SEQ ID NO:85, but excluding the poly(A) tail at the 3′ end of SEQ ID NO:85. Also preferably the polynucleotide isolated according to the above process comprises a nucleotide sequence corresponding to the cDNA sequence of SEQ ID NO:85 from nucleotide 19 to nucleotide 2433, and extending contiguously from a nucleotide sequence corresponding to the 5′ end of said sequence of SEQ ID NO:85 from nucleotide 19 to nucleotide 2433, to a nucleotide sequence corresponding to the 3′ end of said sequence of SEQ ID NO:85 from nucleotide 19 to nucleotide 2433. Also preferably the polynucleotide isolated according to the above process comprises a nucleotide sequence corresponding to the cDNA sequence of SEQ ID NO:85 from nucleotide 70 to nucleotide 2433, and extending contiguously from a nucleotide sequence corresponding to the 5′ end of said sequence of SEQ ID NO:85 from nucleotide 70 to nucleotide 2433, to a nucleotide sequence corresponding to the 3′ end of said sequence of SEQ ID NO:85 from nucleotide 70 to nucleotide 2433. Also preferably the polynucleotide isolated according to the above process comprises a nucleotide sequence corresponding to the cDNA sequence of SEQ ID NO:85 from nucleotide 156 to nucleotide 303, and extending contiguously from a nucleotide sequence corresponding to the 5′ end of said sequence of SEQ ID NO:85 from nucleotide 156 to nucleotide 303, to a nucleotide sequence corresponding to the 3′ end of said sequence of SEQ ID NO:85 from nucleotide 156 to nucleotide 303.

In other embodiments, the present invention provides a composition comprising a protein, wherein said protein comprises an amino acid sequence selected from the group consisting of:
(a) the amino acid sequence of SEQ ID NO:86;
(b) the amino acid sequence of SEQ ID NO:86 from amino acid 47 to amino acid 56;
(c) a fragment of the amino acid sequence of SEQ ID NO:86, the fragment comprising eight contiguous amino acids of SEQ ID NO:86; and
(d) the amino acid sequence encoded by the cDNA insert of clone AU47_{—}8 deposited with the ATCC under accession number 98079;
   the protein being substantially free from other mammalian proteins. Preferably such protein comprises the amino acid sequence of SEQ ID NO:86 or the amino acid sequence of SEQ ID NO:86 from amino acid 47 to amino acid 56. In further preferred embodiments, the present invention provides a protein comprising a fragment of the amino acid sequence of SEQ ID NO:86 having biological activity, the fragment preferably comprising eight (more preferably twenty, most preferably thirty) contiguous amino acids of SEQ ID NO:86, or a protein comprising a fragment of the amino acid sequence of SEQ ID NO:86 having biological activity, the fragment comprising the amino acid sequence from amino acid 397 to amino acid 406 of SEQ ID NO:86.

In one embodiment, the present invention provides a composition comprising an isolated polynucleotide selected from the group consisting of:
(a) a polynucleotide comprising the nucleotide sequence of SEQ ID NO:87;
(b) a polynucleotide comprising the nucleotide sequence of SEQ ID NO:87 from nucleotide 10 to nucleotide 1092;
(c) a polynucleotide comprising the nucleotide sequence of SEQ ID NO:87 from nucleotide 112 to nucleotide 444;
(d) a polynucleotide comprising the nucleotide sequence of the full-length protein coding sequence of clone AU122_{—}1 deposited with the ATCC under accession number 98079;
(e) a polynucleotide encoding the full-length protein encoded by the cDNA insert of clone AU122_{—}1 deposited with the ATCC under accession number 98079;
(f) a polynucleotide comprising the nucleotide sequence of a mature protein coding sequence of clone AU122_{—}1 deposited with the ATCC under accession number 98079;
(g) a polynucleotide encoding a mature protein encoded by the cDNA insert of clone AU122_{—}1 deposited with the ATCC under accession number 98079;
(h) a polynucleotide encoding a protein comprising the amino acid sequence of SEQ ID NO:88;
(i) a polynucleotide encoding a protein comprising a fragment of the amino acid sequence of SEQ ID NO:88 having biological activity, the fragment comprising eight contiguous amino acids of SEQ ID NO:88;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:87.

Preferably, such polynucleotide comprises the nucleotide sequence of SEQ ID NO:87 from nucleotide 10 to nucleotide 1092; the nucleotide sequence of SEQ ID NO:87 from nucleotide 112 to nucleotide 444; the nucleotide sequence of the full-length protein coding sequence of clone AU122_{—}1 deposited with the ATCC under accession number 98079; or the nucleotide sequence of a mature protein coding sequence of clone AU122_{—}1 deposited with the ATCC under accession number 98079. In other preferred embodiments, the polynucleotide encodes the full-length or a mature protein encoded by the cDNA insert of clone AU122_{—}1 deposited with the ATCC under accession number 98079. In yet other preferred embodiments, the present invention provides a polynucleotide encoding a protein comprising the amino acid sequence of SEQ ID NO:88 from amino acid 1 to amino acid 131. In further preferred embodiments, the present invention provides a polynucleotide encoding a protein comprising a fragment of the amino acid sequence of SEQ ID NO:88 having biological activity, the fragment preferably comprising eight (more preferably twenty, most preferably thirty) contiguous amino acids of SEQ ID NO:88, or a polynucleotide encoding a protein comprising a fragment of the amino acid sequence of SEQ ID NO:88 having biological activity, the fragment comprising the amino acid sequence from amino acid 175 to amino acid 184 of SEQ ID NO:88.

Other embodiments provide the gene corresponding to the cDNA sequence of SEQ ID NO:87.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:87, but excluding the poly(A) tail at the 3′ end of SEQ ID NO:87; and
      (ab) the nucleotide sequence of the cDNA insert of clone AU122_{—}1 deposited with the ATCC under accession number 98079;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:87, but excluding the poly(A) tail at the 3′ end of SEQ ID NO:87; and
      (bb) the nucleotide sequence of the cDNA insert of clone AU122_{—}1 deposited with the ATCC under accession number 98079;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:87, and extending contiguously from a nucleotide sequence corresponding to the 5′ end of SEQ ID NO:87 to a nucleotide sequence corresponding to the 3′ end of SEQ ID NO:87, but excluding the poly(A) tail at the 3′ end of SEQ ID NO:87. Also preferably the polynucleotide isolated according to the above process comprises a nucleotide sequence corresponding to the cDNA sequence of SEQ ID NO:87 from nucleotide 10 to nucleotide 1092, and extending contiguously from a nucleotide sequence corresponding to the 5′ end of said sequence of SEQ ID NO:87 from nucleotide 10 to nucleotide 1092, to a nucleotide sequence corresponding to the 3′ end of said sequence of SEQ ID NO:87 from nucleotide 10 to nucleotide 1092. Also preferably the polynucleotide isolated according to the above process comprises a nucleotide sequence corresponding to the cDNA sequence of SEQ ID NO:87 from nucleotide 112 to nucleotide 444, and extending contiguously from a nucleotide sequence corresponding to the 5′ end of said sequence of SEQ ID NO:87 from nucleotide 112 to nucleotide 444, to a nucleotide sequence corresponding to the 3′ end of said sequence of SEQ ID NO:87 from nucleotide 112 to nucleotide 444.

In other embodiments, the present invention provides a composition comprising a protein, wherein said protein comprises an amino acid sequence selected from the group consisting of:
(a) the amino acid sequence of SEQ ID NO:88;
(b) the amino acid sequence of SEQ ID NO:88 from amino acid 1 to amino acid 131;
(c) a fragment of the amino acid sequence of SEQ ID NO:88, the fragment comprising eight contiguous amino acids of SEQ ID NO:88; and
(d) the amino acid sequence encoded by the cDNA insert of clone AU122_{—}1 deposited with the ATCC under accession number 98079;
   the protein being substantially free from other mammalian proteins. Preferably such protein comprises the amino acid sequence of SEQ ID NO:88 or the amino acid sequence of SEQ ID NO:88 from amino acid 1 to amino acid 131. In further preferred embodiments, the present invention provides a protein comprising a fragment of the amino acid sequence of SEQ ID NO:88 having biological activity, the fragment preferably comprising eight (more preferably twenty, most preferably thirty) contiguous amino acids of SEQ ID NO:88, or a protein comprising a fragment of the amino acid sequence of SEQ ID NO:88 having biological activity, the fragment comprising the amino acid sequence from amino acid 175 to amino acid 184 of SEQ ID NO:88.

In one embodiment, the present invention provides a composition comprising an isolated polynucleotide selected from the group consisting of:
(a) a polynucleotide comprising the nucleotide sequence of SEQ ID NO:89;
(b) a polynucleotide comprising the nucleotide sequence of SEQ ID NO:89 from nucleotide 41 to nucleotide 997;
(c) a polynucleotide comprising the nucleotide sequence of SEQ ID NO:89 from nucleotide 41 to nucleotide 853;
(d) a polynucleotide comprising the nucleotide sequence of the full-length protein coding sequence of clone BF208_{—}1 deposited with the ATCC under accession number 98079;
(e) a polynucleotide encoding the full-length protein encoded by the cDNA insert of clone BF208_{—}1 deposited with the ATCC under accession number 98079;
(f) a polynucleotide comprising the nucleotide sequence of a mature protein coding sequence of clone BF208_{—}1 deposited with the ATCC under accession number 98079;
(g) a polynucleotide encoding a mature protein encoded by the cDNA insert of clone BF208_{—}1 deposited with the ATCC under accession number 98079;
(h) a polynucleotide encoding a protein comprising the amino acid sequence of SEQ ID NO:90;
(i) a polynucleotide encoding a protein comprising a fragment of the amino acid sequence of SEQ ID NO:90 having biological activity, the fragment comprising eight contiguous amino acids of SEQ ID NO:90;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:89.

Preferably, such polynucleotide comprises the nucleotide sequence of SEQ ID NO:89 from nucleotide 41 to nucleotide 997; the nucleotide sequence of SEQ ID NO:89 from nucleotide 41 to nucleotide 853; the nucleotide sequence of the full-length protein coding sequence of clone BF208_{—}1 deposited with the ATCC under accession number 98079; or the nucleotide sequence of a mature protein coding sequence of clone BF208_{—}1 deposited with the ATCC under accession number 98079. In other preferred embodiments, the polynucleotide encodes the full-length or a mature protein encoded by the cDNA insert of clone BF208_{—}1 deposited with the ATCC under accession number 98079. In yet other preferred embodiments, the present invention provides a polynucleotide encoding a protein comprising the amino acid sequence of SEQ ID NO:90 from amino acid 229 to amino acid 271, or a polynucleotide encoding a protein comprising the amino acid sequence of SEQ ID NO:90 from amino acid 1 to amino acid 271. In further preferred embodiments, the present invention provides a polynucleotide encoding a protein comprising a fragment of the amino acid sequence of SEQ ID NO:90 having biological activity, the fragment preferably comprising eight (more preferably twenty, most preferably thirty) contiguous amino acids of SEQ ID NO:90, or a polynucleotide encoding a protein comprising a fragment of the amino acid sequence of SEQ ID NO:90 having biological activity, the fragment comprising the amino acid sequence from amino acid 154 to amino acid 163 of SEQ ID NO:90.

Other embodiments provide the gene corresponding to the cDNA sequence of SEQ ID NO:89 and SEQ ID NO:91.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:89;
      (ab) SEQ ID NO:91, but excluding the poly(A) tail at the 3′ end of SEQ ID NO:91; and
      (ac) the nucleotide sequence of the cDNA insert of clone BF208_{—}1 deposited with the ATCC under accession number 98079;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:89;
      (bb) SEQ ID NO:91, but excluding the poly(A) tail at the 3′ end of SEQ ID NO:91; and
      (bc) the nucleotide sequence of the cDNA insert of clone BF208_{—}1 deposited with the ATCC under accession number 98079;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:89 and SEQ ID NO:91, and extending contiguously from a nucleotide sequence corresponding to the 5′ end of SEQ ID NO:89 to a nucleotide sequence corresponding to the 3′ end of SEQ ID NO:91, but excluding the poly(A) tail at the 3′ end of SEQ ID NO:91. Also preferably the polynucleotide isolated according to the above process comprises a nucleotide sequence corresponding to the cDNA sequence of SEQ ID NO:89, and extending contiguously from a nucleotide sequence corresponding to the 5′ end of SEQ ID NO:89 to a nucleotide sequence corresponding to the 3′ end of SEQ ID NO:89. Also preferably the polynucleotide isolated according to the above process comprises a nucleotide sequence corresponding to the cDNA sequence of SEQ ID NO:89 from nucleotide 41 to nucleotide 997, and extending contiguously from a nucleotide sequence corresponding to the 5′ end of said sequence of SEQ ID NO:89 from nucleotide 41 to nucleotide 997, to a nucleotide sequence corresponding to the 3′ end of said sequence of SEQ ID NO:89 from nucleotide 41 to nucleotide 997. Also preferably the polynucleotide isolated according to the above process comprises a nucleotide sequence corresponding to the cDNA sequence of SEQ ID NO:89 from nucleotide 41 to nucleotide 853, and extending contiguously from a nucleotide sequence corresponding to the 5′ end of said sequence of SEQ ID NO:89 from nucleotide 41 to nucleotide 853, to a nucleotide sequence corresponding to the 3′ end of said sequence of SEQ ID NO:89 from nucleotide 41 to nucleotide 853.

In other embodiments, the present invention provides a composition comprising a protein, wherein said protein comprises an amino acid sequence selected from the group consisting of:
(a) the amino acid sequence of SEQ ID NO:90;
(b) the amino acid sequence of SEQ ID NO:90 from amino acid 229 to amino acid 271;
(c) a fragment of the amino acid sequence of SEQ ID NO:90, the fragment comprising eight contiguous amino acids of SEQ ID NO:90; and
(d) the amino acid sequence encoded by the cDNA insert of clone BF208_{—}1 deposited with the ATCC under accession number 98079;
   the protein being substantially free from other mammalian proteins. Preferably such protein comprises the amino acid sequence of SEQ ID NO:90, the amino acid sequence of SEQ ID NO:90 from amino acid 229 to amino acid 271, or the amino acid sequence of SEQ ID NO:90 from amino acid 1 to amino acid 271. In further preferred embodiments, the present invention provides a protein comprising a fragment of the amino acid sequence of SEQ ID NO:90 having biological activity, the fragment preferably comprising eight (more preferably twenty, most preferably thirty) contiguous amino acids of SEQ ID NO:90, or a protein comprising a fragment of the amino acid sequence of SEQ ID NO:90 having biological activity, the fragment comprising the amino acid sequence from amino acid 154 to amino acid 163 of SEQ ID NO:90.

In one embodiment, the present invention provides a composition comprising an isolated polynucleotide selected from the group consisting of:
(a) a polynucleotide comprising the nucleotide sequence of SEQ ID NO:92;
(b) a polynucleotide comprising the nucleotide sequence of SEQ ID NO:92 from nucleotide 1314 to nucleotide 1568;
(c) a polynucleotide comprising the nucleotide sequence of SEQ ID NO:92 from nucleotide 1788 to nucleotide 2049;
(d) a polynucleotide comprising the nucleotide sequence of the full-length protein coding sequence of clone BG513_{—}19 deposited with the ATCC under accession number 98079;
(e) a polynucleotide encoding the full-length protein encoded by the cDNA insert of clone BG513_{—}19 deposited with the ATCC under accession number 98079;
(f) a polynucleotide comprising the nucleotide sequence of a mature protein coding sequence of clone BG513_{—}19 deposited with the ATCC under accession number 98079;
(g) a polynucleotide encoding a mature protein encoded by the cDNA insert of clone BG513_{—}19 deposited with the ATCC under accession number 98079;
(h) a polynucleotide encoding a protein comprising the amino acid sequence of SEQ ID NO:93;
(i) a polynucleotide encoding a protein comprising a fragment of the amino acid sequence of SEQ ID NO:93 having biological activity, the fragment comprising eight contiguous amino acids of SEQ ID NO:93;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:92.

Preferably, such polynucleotide comprises the nucleotide sequence of SEQ ID NO:92 from nucleotide 1314 to nucleotide 1568; the nucleotide sequence of SEQ ID NO:92 from nucleotide 1788 to nucleotide 2049; the nucleotide sequence of the full-length protein coding sequence of clone BG513_{—}19 deposited with the ATCC under accession number 98079; or the nucleotide sequence of a mature protein coding sequence of clone BG513_{—}19 deposited with the ATCC under accession number 98079. In other preferred embodiments, the polynucleotide encodes the full-length or a mature protein encoded by the cDNA insert of clone BG513_{—}19 deposited with the ATCC under accession number 98079. In further preferred embodiments, the present invention provides a polynucleotide encoding a protein comprising a fragment of the amino acid sequence of SEQ ID NO:93 having biological activity, the fragment preferably comprising eight (more preferably twenty, most preferably thirty) contiguous amino acids of SEQ ID NO:93, or a polynucleotide encoding a protein comprising a fragment of the amino acid sequence of SEQ ID NO:93 having biological activity, the fragment comprising the amino acid sequence from amino acid 37 to amino acid 46 of SEQ ID NO:93.

Other embodiments provide the gene corresponding to the cDNA sequence of SEQ ID NO:92.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:92, but excluding the poly(A) tail at the 3′ end of SEQ ID NO:92; and
      (ab) the nucleotide sequence of the cDNA insert of clone BG513_{—}19 deposited with the ATCC under accession number 98079;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:92, but excluding the poly(A) tail at the 3′ end of SEQ ID NO:92; and
      (bb) the nucleotide sequence of the cDNA insert of clone BG513_{—}19 deposited with the ATCC under accession number 98079;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:92, and extending contiguously from a nucleotide sequence corresponding to the 5′ end of SEQ ID NO:92 to a nucleotide sequence corresponding to the 3′ end of SEQ ID NO:92, but excluding the poly(A) tail at the 3′ end of SEQ ID NO:92. Also preferably the polynucleotide isolated according to the above process comprises a nucleotide sequence corresponding to the cDNA sequence of SEQ ID NO:92 from nucleotide 1314 to nucleotide 1568, and extending contiguously from a nucleotide sequence corresponding to the 5′ end of said sequence of SEQ ID NO:92 from nucleotide 1314 to nucleotide 1568, to a nucleotide sequence corresponding to the 3′ end of said sequence of SEQ ID NO:92 from nucleotide 1314 to nucleotide 1568. Also preferably the polynucleotide isolated according to the above process comprises a nucleotide sequence corresponding to the cDNA sequence of SEQ ID NO:92 from nucleotide 1788 to nucleotide 2049, and extending contiguously from a nucleotide sequence corresponding to the 5′ end of said sequence of SEQ ID NO:92 from nucleotide 1788 to nucleotide 2049, to a nucleotide sequence corresponding to the 3′ end of said sequence of SEQ ID NO:92 from nucleotide 1788 to nucleotide 2049.

In other embodiments, the present invention provides a composition comprising a protein, wherein said protein comprises an amino acid sequence selected from the group consisting of:
(a) the amino acid sequence of SEQ ID NO:93;
(b) a fragment of the amino acid sequence of SEQ ID NO:93, the fragment comprising eight contiguous amino acids of SEQ ID NO:93; and
(c) the amino acid sequence encoded by the cDNA insert of clone BG513_{—}19 deposited with the ATCC under accession number 98079;
   the protein being substantially free from other mammalian proteins. Preferably such protein comprises the amino acid sequence of SEQ ID NO:93. In further preferred embodiments, the present invention provides a protein comprising a fragment of the amino acid sequence of SEQ ID NO:93 having biological activity, the fragment preferably comprising eight (more preferably twenty, most preferably thirty) contiguous amino acids of SEQ ID NO:93, or a protein comprising a fragment of the amino acid sequence of SEQ ID NO:93 having biological activity, the fragment comprising the amino acid sequence from amino acid 37 to amino acid 46 of SEQ ID NO:93.

In one embodiment, the present invention provides a composition comprising an isolated polynucleotide selected from the group consisting of:
(a) a polynucleotide comprising the nucleotide sequence of SEQ ID NO:95;
(b) a polynucleotide comprising the nucleotide sequence of SEQ ID NO:95 from nucleotide 210 to nucleotide 530;
(c) a polynucleotide comprising the nucleotide sequence of SEQ ID NO:95 from nucleotide 327 to nucleotide 530;
(d) a polynucleotide comprising the nucleotide sequence of SEQ ID NO:95 from nucleotide 210 to nucleotide 470;
(e) a polynucleotide comprising the nucleotide sequence of the full-length protein coding sequence of clone BG556_{—}8 deposited with the ATCC under accession number 98079;
(f) a polynucleotide encoding the full-length protein encoded by the cDNA insert of clone BG556_{—}8 deposited with the ATCC under accession number 98079;
(g) a polynucleotide comprising the nucleotide sequence of a mature protein coding sequence of clone BG556_{—}8 deposited with the ATCC under accession number 98079;
(h) a polynucleotide encoding a mature protein encoded by the cDNA insert of clone BG556_{—}8 deposited with the ATCC under accession number 98079;
(i) a polynucleotide encoding a protein comprising the amino acid sequence of SEQ ID NO:96;
(j) a polynucleotide encoding a protein comprising a fragment of the amino acid sequence of SEQ ID NO:96 having biological activity, the fragment comprising eight contiguous amino acids of SEQ ID NO:96;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:95.

Preferably, such polynucleotide comprises the nucleotide sequence of SEQ ID NO:95 from nucleotide 210 to nucleotide 530; the nucleotide sequence of SEQ ID NO:95 from nucleotide 327 to nucleotide 530; the nucleotide sequence of SEQ ID NO:95 from nucleotide 210 to nucleotide 470; the nucleotide sequence of the full-length protein coding sequence of clone BG556_{—}8 deposited with the ATCC under accession number 98079; or the nucleotide sequence of a mature protein coding sequence of clone BG556_{—}8 deposited with the ATCC under accession number 98079. In other preferred embodiments, the polynucleotide encodes the full-length or a mature protein encoded by the cDNA insert of clone BG556_{—}8 deposited with the ATCC under accession number 98079. In yet other preferred embodiments, the present invention provides a polynucleotide encoding a protein comprising the amino acid sequence of SEQ ID NO:96 from amino acid 29 to amino acid 87, or a polynucleotide encoding a protein comprising the amino acid sequence of SEQ ID NO:96 from amino acid 1 to amino acid 87. In further preferred embodiments, the present invention provides a polynucleotide encoding a protein comprising a fragment of the amino acid sequence of SEQ ID NO:96 having biological activity, the fragment preferably comprising eight (more preferably twenty, most preferably thirty) contiguous amino acids of SEQ ID NO:96, or a polynucleotide encoding a protein comprising a fragment of the amino acid sequence of SEQ ID NO:96 having biological activity, the fragment comprising the amino acid sequence from amino acid 48 to amino acid 57 of SEQ ID NO:96.

Other embodiments provide the gene corresponding to the cDNA sequence of SEQ ID NO:95 and SEQ ID NO:94.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:94;
      (ab) SEQ ID NO:95; and
      (ac) the nucleotide sequence of the cDNA insert of clone BG556_{—}8 deposited with the ATCC under accession number 98079;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:94;
      (bb) SEQ ID NO:95; and
      (bc) the nucleotide sequence of the cDNA insert of clone BG556_{—}8 deposited with the ATCC under accession number 98079;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:94 and SEQ ID NO:95, and extending contiguously from a nucleotide sequence corresponding to the 5′ end of SEQ ID NO:94 to a nucleotide sequence corresponding to the 3′ end of SEQ ID NO:95. Also preferably the polynucleotide isolated according to the above process comprises a nucleotide sequence corresponding to the cDNA sequence of SEQ ID NO:95, and extending contiguously from a nucleotide sequence corresponding to the 5′ end of SEQ ID NO:95 to a nucleotide sequence corresponding to the 3′ end of SEQ ID NO:95. Also preferably the polynucleotide isolated according to the above process comprises a nucleotide sequence corresponding to the cDNA sequence of SEQ ID NO:95 from nucleotide 210 to nucleotide 530, and extending contiguously from a nucleotide sequence corresponding to the 5′ end of said sequence of SEQ ID NO:95 from nucleotide 210 to nucleotide 530, to a nucleotide sequence corresponding to the 3′ end of said sequence of SEQ ID NO:95 from nucleotide 210 to nucleotide 530. Also preferably the polynucleotide isolated according to the above process comprises a nucleotide sequence corresponding to the cDNA sequence of SEQ ID NO:95 from nucleotide 327 to nucleotide 530, and extending contiguously from a nucleotide sequence corresponding to the 5′ end of said sequence of SEQ ID NO:95 from nucleotide 327 to nucleotide 530, to a nucleotide sequence corresponding to the 3′ end of said sequence of SEQ ID NO:95 from nucleotide 327 to nucleotide 530. Also preferably the polynucleotide isolated according to the above process comprises a nucleotide sequence corresponding to the cDNA sequence of SEQ ID NO:95 from nucleotide 210 to nucleotide 470, and extending contiguously from a nucleotide sequence corresponding to the 5′ end of said sequence of SEQ ID NO:95 from nucleotide 210 to nucleotide 470, to a nucleotide sequence corresponding to the 3′ end of said sequence of SEQ ID NO:95 from nucleotide 210 to nucleotide 470.

In other embodiments, the present invention provides a composition comprising a protein, wherein said protein comprises an amino acid sequence selected from the group consisting of:
(a) the amino acid sequence of SEQ ID NO:96;
(b) the amino acid sequence of SEQ ID NO:96 from amino acid 29 to amino acid 87;
(c) a fragment of the amino acid sequence of SEQ ID NO:96, the fragment comprising eight contiguous amino acids of SEQ ID NO:96; and
(d) the amino acid sequence encoded by the cDNA insert of clone BG556_{—}8 deposited with the ATCC under accession number 98079;
   the protein being substantially free from other mammalian proteins. Preferably such protein comprises the amino add sequence of SEQ ID NO:96, the amino acid sequence of SEQ ID NO:96 from amino acid 29 to amino acid 87, or the amino acid sequence of SEQ ID NO:96 from amino acid 1 to amino acid 87. In further preferred embodiments, the present invention provides a protein comprising a fragment of the amino acid sequence of SEQ ID NO:96 having biological activity, the fragment preferably comprising eight (more preferably twenty, most preferably thirty) contiguous amino acids of SEQ ID NO:96, or a protein comprising a fragment of the amino acid sequence of SEQ ID NO:96 having biological activity, the fragment comprising the amino acid sequence from amino acid 48 to amino acid 57 of SEQ ID NO:96.

In one embodiment, the present invention provides a composition comprising an isolated polynucleotide selected from the group consisting of:
(a) a polynucleotide comprising the nucleotide sequence of SEQ ID NO:97;
(b) a polynucleotide comprising the nucleotide sequence of SEQ ID NO:97 from nucleotide 14 to nucleotide 433;
(c) a polynucleotide comprising the nucleotide sequence of the full-length protein coding sequence of clone C195_{—}1 deposited with the ATCC under accession number 98079;
(d) a polynucleotide encoding the full-length protein encoded by the cDNA insert of clone C195_{—}1 deposited with the ATCC under accession number 98079;
(e) a polynucleotide comprising the nucleotide sequence of a mature protein coding sequence of clone C195_{—}1 deposited with the ATCC under accession number 98079;
(f) a polynucleotide encoding a mature protein encoded by the cDNA insert of clone C195_{—}1 deposited with the ATCC under accession number 98079;
(g) a polynucleotide encoding a protein comprising the amino acid sequence of SEQ ID NO:98;
(h) a polynucleotide encoding a protein comprising a fragment of the amino acid sequence of SEQ ID NO:98 having biological activity, the fragment comprising eight contiguous amino acids of SEQ ID NO:98;
(i) a polynucleotide which is an allelic variant of a polynucleotide of (a)-(f) above;
(j) a polynucleotide which encodes a species homologue of the protein of (g) or (h) above;
(k) a polynucleotide that hybridizes under stringent conditions to any one of the polynucleotides specified in (a)-(h); and
(l) a polynucleotide that hybridizes under stringent conditions to any one of the polynucleotides specified in (a)-(h) and that has a length that is at least 25% of the length of SEQ ID NO:97.

Preferably, such polynucleotide comprises the nucleotide sequence of SEQ ID NO:97 from nucleotide 14 to nucleotide 433; the nucleotide sequence of the full-length protein coding sequence of clone C195_{—}1 deposited with the ATCC under accession number 98079; or the nucleotide sequence of a mature protein coding sequence of clone C195_{—}1 deposited with the ATCC under accession number 98079. In other preferred embodiments, the polynucleotide encodes the full-length or a mature protein encoded by the cDNA insert of clone C195_{—}1 deposited with the ATCC under accession number 98079. In yet other preferred embodiments, the present invention provides a polynucleotide encoding a protein comprising the amino acid sequence of SEQ ID NO:98 from amino acid 52 to amino acid 140. In further preferred embodiments, the present invention provides a polynucleotide encoding a protein comprising a fragment of the amino acid sequence of SEQ ID NO:98 having biological activity, the fragment preferably comprising eight (more preferably twenty, most preferably thirty) contiguous amino acids of SEQ ID NO:98, or a polynucleotide encoding a protein comprising a fragment of the amino acid sequence of SEQ ID NO:98 having biological activity, the fragment comprising the amino acid sequence from amino acid 65 to amino acid 74 of SEQ ID NO:98.

Other embodiments provide the gene corresponding to the cDNA sequence of SEQ ID NO:97 and SEQ ID NO:99.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:97;
      (ab) SEQ ID NO:99, but excluding the poly(A) tail at the 3′ end of SEQ ID NO:99; and
      (ac) the nucleotide sequence of the cDNA insert of clone C195_{—}1 deposited with the ATCC under accession number 98079;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:97;
      (bb) SEQ ID NO:99, but excluding the poly(A) tail at the 3′ end of SEQ ID NO:99; and
      (bc) the nucleotide sequence of the cDNA insert of clone C195_{—}1 deposited with the ATCC under accession number 98079;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:97 and SEQ ID NO:99, and extending contiguously from a nucleotide sequence corresponding to the 5′ end of SEQ ID NO:97 to a nucleotide sequence corresponding to the 3′ end of SEQ ID NO:99, but excluding the poly(A) tail at the 3′ end of SEQ ID NO:99. Also preferably the polynucleotide isolated according to the above process comprises a nucleotide sequence corresponding to the cDNA sequence of SEQ ID NO:97, and extending contiguously from a nucleotide sequence corresponding to the 5′ end of SEQ ID NO:97 to a nucleotide sequence corresponding to the 3′ end of SEQ ID NO:97. Also preferably the polynucleotide isolated according to the above process comprises a nucleotide sequence corresponding to the cDNA sequence of SEQ ID NO:97 from nucleotide 14 to nucleotide 433, and extending contiguously from a nucleotide sequence corresponding to the 5′ end of said sequence of SEQ ID NO:97 from nucleotide 14 to nucleotide 433, to a nucleotide sequence corresponding to the 3′ end of said sequence of SEQ ID NO:97 from nucleotide 14 to nucleotide 433.

In other embodiments, the present invention provides a composition comprising a protein, wherein said protein comprises an amino acid sequence selected from the group consisting of:
(a) the amino acid sequence of SEQ ID NO:98;
(b) the amino acid sequence of SEQ ID NO:98 from amino acid 52 to amino acid 140;
(c) a fragment of the amino acid sequence of SEQ ID NO:98, the fragment comprising eight contiguous amino acids of SEQ ID NO:98; and
(d) the amino acid sequence encoded by the cDNA insert of clone C195_{—}1 deposited with the ATCC under accession number 98079;
   the protein being substantially free from other mammalian proteins. Preferably such protein comprises the amino acid sequence of SEQ ID NO:98 or the amino acid sequence of SEQ ID NO:98 from amino acid 52 to amino acid 140. In further preferred embodiments, the present invention provides a protein comprising a fragment of the amino acid sequence of SEQ ID NO:98 having biological activity, the fragment preferably comprising eight (more preferably twenty, most preferably thirty) contiguous amino acids of SEQ ID NO:98, or a protein comprising a fragment of the amino acid sequence of SEQ ID NO:98 having biological activity, the fragment comprising the amino acid sequence from amino acid 65 to amino acid 74 of SEQ ID NO:98.

In one embodiment, the present invention provides a composition comprising an isolated polynucleotide selected from the group consisting of:
(a) a polynucleotide comprising the nucleotide sequence of SEQ ID NO:101;
(b) a polynucleotide comprising the nucleotide sequence of SEQ ID NO:101 from nucleotide 14 to nucleotide 733;
(c) a polynucleotide comprising the nucleotide sequence of the full-length protein coding sequence of clone C195_{—}4 deposited with the ATCC under accession number 98182;
(d) a polynucleotide encoding the full-length protein encoded by the cDNA insert of clone C195_{—}4 deposited with the ATCC under accession number 98182;
(e) a polynucleotide comprising the nucleotide sequence of a mature protein coding sequence of clone C195_{—}4 deposited with the ATCC under accession number 98182;
(f) a polynucleotide encoding a mature protein encoded by the cDNA insert of clone C195_{—}4 deposited with the ATCC under accession number 98182;
(g) a polynucleotide encoding a protein comprising the amino acid sequence of SEQ ID NO:102;
(h) a polynucleotide encoding a protein comprising a fragment of the amino acid sequence of SEQ ID NO:102 having biological activity, the fragment comprising eight contiguous amino acids of SEQ ID NO:102;
(i) a polynucleotide which is an allelic variant of a polynucleotide of (a)-(f) above;
(j) a polynucleotide which encodes a species homologue of the protein of (g) or (h) above;
(k) a polynucleotide that hybridizes under stringent conditions to any one of the polynucleotides specified in (a)-(h); and
(l) a polynucleotide that hybridizes under stringent conditions to any one of the polynucleotides specified in (a)-(h) and that has a length that is at least 25% of the length of SEQ ID NO:101.

Preferably, such polynucleotide comprises the nucleotide sequence of SEQ ID NO:101 from nucleotide 14 to nucleotide 733; the nucleotide sequence of the full-length protein coding sequence of clone C195_{—}4 deposited with the ATCC under accession number 98182; or the nucleotide sequence of a mature protein coding sequence of clone C195_{—}4 deposited with the ATCC under accession number 98182. In other preferred embodiments, the polynucleotide encodes the full-length or a mature protein encoded by the cDNA insert of clone C195_{—}4 deposited with the ATCC under accession number 98182. In yet other preferred embodiments, the present invention provides a polynucleotide encoding a protein comprising the amino acid sequence of SEQ ID NO:102 from amino acid 1 to amino acid 140. In further preferred embodiments, the present invention provides a polynucleotide encoding a protein comprising a fragment of the amino acid sequence of SEQ ID NO:102 having biological activity, the fragment preferably comprising eight (more preferably twenty, most preferably thirty) contiguous amino adds of SEQ ID NO:102, or a polynucleotide encoding a protein comprising a fragment of the amino acid sequence of SEQ ID NO:102 having biological activity, the fragment comprising the amino acid sequence from amino acid 115 to amino acid 124 of SEQ ID NO:102. Other embodiments provide the gene corresponding to the cDNA sequence of SEQ ID NO:101, SEQ ID NO:100, and SEQ ID NO:103.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:100;
      (ab) SEQ ID NO:101;
      (ac) SEQ ID NO:103, but excluding the poly(A) tail at the 3′ end of SEQ ID NO:103; and
      (ad) the nucleotide sequence of the cDNA insert of clone C195_{—}4 deposited with the ATCC under accession number 98182;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:100;
      (bb) SEQ ID NO:101;
      (bc) SEQ ID NO:103, but excluding the poly(A) tail at the 3′ end of SEQ ID NO:103; and
      (bd) the nucleotide sequence of the cDNA insert of clone C195_{—}4 deposited with the ATCC under accession number 98182;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:100, SEQ ID NO:101, and SEQ ID NO:103, and extending contiguously from a nucleotide sequence corresponding to the 5′ end of SEQ ID NO:100 to a nucleotide sequence corresponding to the 3′ end of SEQ ID NO:103, but excluding the poly(A) tail at the 3′ end of SEQ ID NO:103. Also preferably the polynucleotide isolated according to the above process comprises a nucleotide sequence corresponding to the cDNA sequence of SEQ ID NO:101, and extending contiguously from a nucleotide sequence corresponding to the 5′ end of SEQ ID NO:101 to a nucleotide sequence corresponding to the 3′ end of SEQ ID NO:101. Also preferably the polynucleotide isolated according to the above process comprises a nucleotide sequence corresponding to the cDNA sequence of SEQ ID NO:101 from nucleotide 14 to nucleotide 733, and extending contiguously from a nucleotide sequence corresponding to the 5′ end of said sequence of SEQ ID NO:101 from nucleotide 14 to nucleotide 733, to a nucleotide sequence corresponding to the 3′ end of said sequence of SEQ ID NO:101 from nucleotide 14 to nucleotide 733.

In other embodiments, the present invention provides a composition comprising a protein, wherein said protein comprises an amino acid sequence selected from the group consisting of:
(a) the amino acid sequence of SEQ ID NO:102;
(b) the amino acid sequence of SEQ ID NO:102 from amino acid 1 to amino acid 140;
(c) a fragment of the amino acid sequence of SEQ ID NO:102, the fragment comprising eight contiguous amino acids of SEQ ID NO:102; and
(d) the amino acid sequence encoded by the cDNA insert of clone C195_{—}4 deposited with the ATCC under accession number 98182;
   the protein being substantially free from other mammalian proteins. Preferably such protein comprises the amino acid sequence of SEQ ID NO:102 or the amino acid sequence of SEQ ID NO:102 from amino acid 1 to amino acid 140. In further preferred embodiments, the present invention provides a protein comprising a fragment of the amino acid sequence of SEQ ID NO:102 having biological activity, the fragment preferably comprising eight (more preferably twenty, most preferably thirty) contiguous amino acids of SEQ ID NO:102, or a protein comprising a fragment of the amino acid sequence of SEQ ID NO:102 having biological activity, the fragment comprising the amino acid sequence from amino acid 115 to amino acid 124 of SEQ ID NO:102.

In one embodiment, the present invention provides a composition comprising an isolated polynucleotide selected from the group consisting of:
(a) a polynucleotide comprising the nucleotide sequence of SEQ ID NO:104;
(b) a polynucleotide comprising the nucleotide sequence of SEQ ID NO:104 from nucleotide 506 to nucleotide 736;
(c) a polynucleotide comprising the nucleotide sequence of SEQ ID NO:104 from nucleotide 561 to nucleotide 710;
(d) a polynucleotide comprising the nucleotide sequence of the full-length protein coding sequence of clone O276_{—}16 deposited with the ATCC under accession number 98079;
(e) a polynucleotide encoding the full-length protein encoded by the cDNA insert of clone O276_{—}16 deposited with the ATCC under accession number 98079;
(f) a polynucleotide comprising the nucleotide sequence of a mature protein coding sequence of clone O276_{—}16 deposited with the ATCC under accession number 98079;
(g) a polynucleotide encoding a mature protein encoded by the cDNA insert of clone O276_{—}16 deposited with the ATCC under accession number 98079;
(h) a polynucleotide encoding a protein comprising the amino acid sequence of SEQ ID NO:105;
(i) a polynucleotide encoding a protein comprising a fragment of the amino acid sequence of SEQ ID NO:105 having biological activity, the fragment comprising eight contiguous amino acids of SEQ ID NO:105;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:104.

Preferably, such polynucleotide comprises the nucleotide sequence of SEQ ID NO:104 from nucleotide 506 to nucleotide 736; the nucleotide sequence of SEQ ID NO:104 from nucleotide 561 to nucleotide 710; the nucleotide sequence of the full-length protein coding sequence of clone O276_{—}16 deposited with the ATCC under accession number 98079; or the nucleotide sequence of a mature protein coding sequence of clone O276_{—}16 deposited with the ATCC under accession number 98079. In other preferred embodiments, the polynucleotide encodes the full-length or a mature protein encoded by the cDNA insert of clone O276_{—}16 deposited with the ATCC under accession number 98079. In further preferred embodiments, the present invention provides a polynucleotide encoding a protein comprising a fragment of the amino acid sequence of SEQ ID NO:105 having biological activity, the fragment preferably comprising eight (more preferably twenty, most preferably thirty) contiguous amino acids of SEQ ID NO:105, or a polynucleotide encoding a protein comprising a fragment of the amino acid sequence of SEQ ID NO:105 having biological activity, the fragment comprising the amino acid sequence from amino acid 33 to amino acid 42 of SEQ ID NO:105.

Other embodiments provide the gene corresponding to the cDNA sequence of SEQ ID NO:104.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:104; and
      (ab) the nucleotide sequence of the cDNA insert of clone O276_{—}16 deposited with the ATCC under accession number 98079;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:104; and
      (bb) the nucleotide sequence of the cDNA insert of clone O276_{—}16 deposited with the ATCC under accession number 98079;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:104, and extending contiguously from a nucleotide sequence corresponding to the 5′ end of SEQ ID NO:104 to a nucleotide sequence corresponding to the 3′ end of SEQ ID NO:104. Also preferably the polynucleotide isolated according to the above process comprises a nucleotide sequence corresponding to the cDNA sequence of SEQ ID NO:104 from nucleotide 506 to nucleotide 736, and extending contiguously from a nucleotide sequence corresponding to the 5′ end of said sequence of SEQ ID NO:104 from nucleotide 506 to nucleotide 736, to a nucleotide sequence corresponding to the 3′ end of said sequence of SEQ ID NO:104 from nucleotide 506 to nucleotide 736. Also preferably the polynucleotide isolated according to the above process comprises a nucleotide sequence corresponding to the cDNA sequence of SEQ ID NO:104 from nucleotide 561 to nucleotide 710, and extending contiguously from a nucleotide sequence corresponding to the 5′ end of said sequence of SEQ ID NO:104 from nucleotide 561 to nucleotide 710, to a nucleotide sequence corresponding to the 3′ end of said sequence of SEQ ID NO:104 from nucleotide 561 to nucleotide 710.

In other embodiments, the present invention provides a composition comprising a protein, wherein said protein comprises an amino acid sequence selected from the group consisting of:
(a) the amino acid sequence of SEQ ID NO:105;
(b) a fragment of the amino acid sequence of SEQ ID NO:105, the fragment comprising eight contiguous amino acids of SEQ ID NO:105; and
(c) the amino acid sequence encoded by the cDNA insert of clone O276_{—}16 deposited with the ATCC under accession number 98079;
   the protein being substantially free from other mammalian proteins. Preferably such protein comprises the amino acid sequence of SEQ ID NO:105. In further preferred embodiments, the present invention provides a protein comprising a fragment of the amino acid sequence of SEQ ID NO:105 having biological activity, the fragment preferably comprising eight (more preferably twenty, most preferably thirty) contiguous amino acids of SEQ ID NO:105, or a protein comprising a fragment of the amino acid sequence of SEQ ID NO:105 having biological activity, the fragment comprising the amino acid sequence from amino acid 33 to amino acid 42 of SEQ ID NO:105.

In one embodiment, the present invention provides a composition comprising an isolated polynucleotide selected from the group consisting of:
(a) a polynucleotide comprising the nucleotide sequence of SEQ ID NO:106;
(b) a polynucleotide comprising the nucleotide sequence of SEQ ID NO:106 from nucleotide 113 to nucleotide 742;
(c) a polynucleotide comprising the nucleotide sequence of SEQ ID NO:106 from nucleotide 179 to nucleotide 742;
(d) a polynucleotide comprising the nucleotide sequence of SEQ ID NO:106 from nucleotide 224 to nucleotide 379;
(e) a polynucleotide comprising the nucleotide sequence of the full-length protein coding sequence of clone AC41_{—}1 deposited with the ATCC under accession number 98101;
(f) a polynucleotide encoding the full-length protein encoded by the cDNA insert of clone AC41_{—}1 deposited with the ATCC under accession number 98101;
(g) a polynucleotide comprising the nucleotide sequence of a mature protein coding sequence of clone AC41_{—}1 deposited with the ATCC under accession number 98101;
(h) a polynucleotide encoding a mature protein encoded by the cDNA insert of clone AC41_{—}1 deposited with the ATCC under accession number 98101;
(i) a polynucleotide encoding a protein comprising the amino acid sequence of SEQ ID NO:107;
(j) a polynucleotide encoding a protein comprising a fragment of the amino acid sequence of SEQ ID NO:107 having biological activity, the fragment comprising eight contiguous amino acids of SEQ ID NO:107;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:106.

Preferably, such polynucleotide comprises the nucleotide sequence of SEQ ID NO:106 from nucleotide 113 to nucleotide 742; the nucleotide sequence of SEQ ID NO:106 from nucleotide 179 to nucleotide 742; the nucleotide sequence of SEQ ID NO:106 from nucleotide 224 to nucleotide 379; the nucleotide sequence of the full-length protein coding sequence of clone AC41_{—}1 deposited with the ATCC under accession number 98101; or the nucleotide sequence of a mature protein coding sequence of clone AC41_{—}1 deposited with the ATCC under accession number 98101. In other preferred embodiments, the polynucleotide encodes the full-length or a mature protein encoded by the cDNA insert of clone AC41_{—}1 deposited with the ATCC under accession number 98101. In yet other preferred embodiments, the present invention provides a polynucleotide encoding a protein comprising the amino acid sequence of SEQ ID NO:107 from amino acid 91 to amino acid 129. In further preferred embodiments, the present invention provides a polynucleotide encoding a protein comprising a fragment of the amino acid sequence of SEQ ID NO:107 having biological activity, the fragment preferably comprising eight (more preferably twenty, most preferably thirty) contiguous amino acids of SEQ ID NO:107, or a polynucleotide encoding a protein comprising a fragment of the amino acid sequence of SEQ ID NO:107 having biological activity, the fragment comprising the amino acid sequence from amino acid 100 to amino acid 109 of SEQ ID NO:107.

Other embodiments provide the gene corresponding to the cDNA sequence of SEQ ID NO:106.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:106, but excluding the poly(A) tail at the 3′ end of SEQ ID NO:106; and
      (ab) the nucleotide sequence of the cDNA insert of clone AC41_{—}1 deposited with the ATCC under accession number 98101;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:106, but excluding the poly(A) tail at the 3′ end of SEQ ID NO:106; and
      (bb) the nucleotide sequence of the cDNA insert of clone AC41_{—}1 deposited with the ATCC under accession number 98101;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:106, and extending contiguously from a nucleotide sequence corresponding to the 5′ end of SEQ ID NO:106 to a nucleotide sequence corresponding to the 3′ end of SEQ ID NO:106, but excluding the poly(A) tail at the 3′ end of SEQ ID NO:106. Also preferably the polynucleotide isolated according to the above process comprises a nucleotide sequence corresponding to the cDNA sequence of SEQ ID NO:106 from nucleotide 113 to nucleotide 742, and extending contiguously from a nucleotide sequence corresponding to the 5′ end of said sequence of SEQ ID NO:106 from nucleotide 113 to nucleotide 742, to a nucleotide sequence corresponding to the 3′ end of said sequence of SEQ ID NO:106 from nucleotide 113 to nucleotide 742. Also preferably the polynucleotide isolated according to the above process comprises a nucleotide sequence corresponding to the cDNA sequence of SEQ ID NO:106 from nucleotide 179 to nucleotide 742, and extending contiguously from a nucleotide sequence corresponding to the 5′ end of said sequence of SEQ ID NO:106 from nucleotide 179 to nucleotide 742, to a nucleotide sequence corresponding to the 3′ end of said sequence of SEQ ID NO:106 from nucleotide 179 to nucleotide 742. Also preferably the polynucleotide isolated according to the above process comprises a nucleotide sequence corresponding to the cDNA sequence of SEQ ID NO:106 from nucleotide 224 to nucleotide 379, and extending contiguously from a nucleotide sequence corresponding to the 5′ end of said sequence of SEQ ID NO:106 from nucleotide 224 to nucleotide 379, to a nucleotide sequence corresponding to the 3′ end of said sequence of SEQ ID NO:106 from nucleotide 224 to nucleotide 379.

In other embodiments, the present invention provides a composition comprising a protein, wherein said protein comprises an amino acid sequence selected from the group consisting of:
(a) the amino acid sequence of SEQ ID NO:107;
(b) the amino acid sequence of SEQ ID NO:107 from amino acid 91 to amino acid 129;
(c) a fragment of the amino acid sequence of SEQ ID NO:107, the fragment comprising eight contiguous amino acids of SEQ ID NO:107; and
(d) the amino acid sequence encoded by the cDNA insert of clone AC41_{—}1 deposited with the ATCC under accession number 98101;
   the protein being substantially free from other mammalian proteins. Preferably such protein comprises the amino acid sequence of SEQ ID NO:107 or the amino acid sequence of SEQ ID NO:107 from amino acid 91 to amino acid 129. In further preferred embodiments, the present invention provides a protein comprising a fragment of the amino acid sequence of SEQ ID NO:107 having biological activity, the fragment preferably comprising eight (more preferably twenty, most preferably thirty) contiguous amino acids of SEQ ID NO:107, or a protein comprising a fragment of the amino acid sequence of SEQ ID NO:107 having biological activity, the fragment comprising the amino acid sequence from amino acid 100 to amino acid 109 of SEQ ID NO:107.

In one embodiment, the present invention provides a composition comprising an isolated polynucleotide selected from the group consisting of:
(a) a polynucleotide comprising the nucleotide sequence of SEQ ID NO:108;
(b) a polynucleotide comprising the nucleotide sequence of SEQ ID NO:108 from nucleotide 161 to nucleotide 1126;
(c) a polynucleotide comprising the nucleotide sequence of SEQ ID NO:108 from nucleotide 218 to nucleotide 1126;
(d) a polynucleotide comprising the nucleotide sequence of SEQ ID NO:108 from nucleotide 219 to nucleotide 553;
(e) a polynucleotide comprising the nucleotide sequence of the full-length protein coding sequence of clone AC222_{—}1 deposited with the ATCC under accession number 98101;
(f) a polynucleotide encoding the full-length protein encoded by the cDNA insert of clone AC222_{—}1 deposited with the ATCC under accession number 98101;
(g) a polynucleotide comprising the nucleotide sequence of a mature protein coding sequence of clone AC222_{—}1 deposited with the ATCC under accession number 98101;
(h) a polynucleotide encoding a mature protein encoded by the cDNA insert of clone AC222_{—}1 deposited with the ATCC under accession number 98101;
(i) a polynucleotide encoding a protein comprising the amino acid sequence of SEQ ID NO:109;
(j) a polynucleotide encoding a protein comprising a fragment of the amino acid sequence of SEQ ID NO:109 having biological activity, the fragment comprising eight contiguous amino acids of SEQ ID NO:109;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:108.

Preferably, such polynucleotide comprises the nucleotide sequence of SEQ ID NO:108 from nucleotide 161 to nucleotide 1126; the nucleotide sequence of SEQ ID NO:108 from nucleotide 218 to nucleotide 1126; the nucleotide sequence of SEQ ID NO:108 from nucleotide 219 to nucleotide 553; the nucleotide sequence of the full-length protein coding sequence of clone AC222_{—}1 deposited with the ATCC under accession number 98101; or the nucleotide sequence of a mature protein coding sequence of clone AC222_{—}1 deposited with the ATCC under accession number 98101. In other preferred embodiments, the polynucleotide encodes the full-length or a mature protein encoded by the cDNA insert of clone AC222_{—}1 deposited with the ATCC under accession number 98101. In yet other preferred embodiments, the present invention provides a polynucleotide encoding a protein comprising the amino acid sequence of SEQ ID NO:109 from amino acid 21 to amino acid 131. In further preferred embodiments, the present invention provides a polynucleotide encoding a protein comprising a fragment of the amino acid sequence of SEQ ID NO:109 having biological activity, the fragment preferably comprising eight (more preferably twenty, most preferably thirty) contiguous amino acids of SEQ ID NO:109, or a polynucleotide encoding a protein comprising a fragment of the amino acid sequence of SEQ ID NO:109 having biological activity, the fragment comprising the amino acid sequence from amino acid 156 to amino acid 165 of SEQ ID NO:109.

Other embodiments provide the gene corresponding to the cDNA sequence of SEQ ID NO:108.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:108, but excluding the poly(A) tail at the 3′ end of SEQ ID NO:108; and
      (ab) the nucleotide sequence of the cDNA insert of clone AC222_{—}1 deposited with the ATCC under accession number 98101;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:108, but excluding the poly(A) tail at the 3′ end of SEQ ID NO:108; and
      (bb) the nucleotide sequence of the cDNA insert of clone AC222_{—}1 deposited with the ATCC under accession number 98101;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:108, and extending contiguously from a nucleotide sequence corresponding to the 5′ end of SEQ ID NO:108 to a nucleotide sequence corresponding to the 3′ end of SEQ ID NO:108, but excluding the poly(A) tail at the 3′ end of SEQ ID NO:108. Also preferably the polynucleotide isolated according to the above process comprises a nucleotide sequence corresponding to the cDNA sequence of SEQ ID NO:108 from nucleotide 161 to nucleotide 1126, and extending contiguously from a nucleotide sequence corresponding to the 5′ end of said sequence of SEQ ID NO:108 from nucleotide 161 to nucleotide 1126, to a nucleotide sequence corresponding to the 3′ end of said sequence of SEQ ID NO:108 from nucleotide 161 to nucleotide 1126. Also preferably the polynucleotide isolated according to the above process comprises a nucleotide sequence corresponding to the cDNA sequence of SEQ ID NO:108 from nucleotide 218 to nucleotide 1126, and extending contiguously from a nucleotide sequence corresponding to the 5′ end of said sequence of SEQ ID NO:108 from nucleotide 218 to nucleotide 1126, to a nucleotide sequence corresponding to the 3′ end of said sequence of SEQ ID NO:108 from nucleotide 218 to nucleotide 1126. Also preferably the polynucleotide isolated according to the above process comprises a nucleotide sequence corresponding to the cDNA sequence of SEQ ID NO:108 from nucleotide 219 to nucleotide 553, and extending contiguously from a nucleotide sequence corresponding to the 5′ end of said sequence of SEQ ID NO:108 from nucleotide 219 to nucleotide 553, to a nucleotide sequence corresponding to the 3′ end of said sequence of SEQ ID NO:108 from nucleotide 219 to nucleotide 553.

In other embodiments, the present invention provides a composition comprising a protein, wherein said protein comprises an amino acid sequence selected from the group consisting of:
(a) the amino acid sequence of SEQ ID NO:109;
(b) the amino acid sequence of SEQ ID NO:109 from amino acid 21 to amino acid 131;
(c) a fragment of the amino acid sequence of SEQ ID NO:109, the fragment comprising eight contiguous amino acids of SEQ ID NO:109; and
(d) the amino acid sequence encoded by the cDNA insert of clone AC222_{—}1 deposited with the ATCC under accession number 98101;
   the protein being substantially free from other mammalian proteins. Preferably such protein comprises the amino acid sequence of SEQ ID NO:109 or the amino acid sequence of SEQ ID NO:109 from amino acid 21 to amino acid 131. In further preferred embodiments, the present invention provides a protein comprising a fragment of the amino acid sequence of SEQ ID NO:109 having biological activity, the fragment preferably comprising eight (more preferably twenty, most preferably thirty) contiguous amino acids of SEQ ID NO:109, or a protein comprising a fragment of the amino acid sequence of SEQ ID NO:109 having biological activity, the fragment comprising the amino acid sequence from amino acid 156 to amino acid 165 of SEQ ID NO:109.

In one embodiment, the present invention provides a composition comprising an isolated polynucleotide selected from the group consisting of:
(a) a polynucleotide comprising the nucleotide sequence of SEQ ID NO:110;
(b) a polynucleotide comprising the nucleotide sequence of SEQ ID NO:110 from nucleotide 827 to nucleotide 994;
(c) a polynucleotide comprising the nucleotide sequence of SEQ ID NO:110 from nucleotide 869 to nucleotide 994;
(d) a polynucleotide comprising the nucleotide sequence of SEQ ID NO:110 from nucleotide 495 to nucleotide 665;
(e) a polynucleotide comprising the nucleotide sequence of the full-length protein coding sequence of clone AJ143_{—}1 deposited with the ATCC under accession number 98101;
(f) a polynucleotide encoding the full-length protein encoded by the cDNA insert of clone AJ143_{—}1 deposited with the ATCC under accession number 98101;
(g) a polynucleotide comprising the nucleotide sequence of a mature protein coding sequence of clone AJ143_{—}1 deposited with the ATCC under accession number 98101;
(h) a polynucleotide encoding a mature protein encoded by the cDNA insert of clone AJ143_{—}1 deposited with the ATCC under accession number 98101;
(i) a polynucleotide encoding a protein comprising the amino acid sequence of SEQ ID NO:111;
(j) a polynucleotide encoding a protein comprising a fragment of the amino acid sequence of SEQ ID NO:111 having biological activity, the fragment comprising eight contiguous amino acids of SEQ ID NO:111;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:110.

Preferably, such polynucleotide comprises the nucleotide sequence of SEQ ID NO:110 from nucleotide 827 to nucleotide 994; the nucleotide sequence of SEQ ID NO:110 from nucleotide 869 to nucleotide 994; the nucleotide sequence of SEQ ID NO:110 from nucleotide 495 to nucleotide 665; the nucleotide sequence of the full-length protein coding sequence of clone AJ143_{—}1 deposited with the ATCC under accession number 98101; or the nucleotide sequence of a mature protein coding sequence of clone AJ143_{—}1 deposited with the ATCC under accession number 98101. In other preferred embodiments, the polynucleotide encodes the full-length or a mature protein encoded by the cDNA insert of clone AJ143_{—}1 deposited with the ATCC under accession number 98101. In yet other preferred embodiments, the present invention provides a polynucleotide encoding a protein comprising the amino acid sequence of SEQ ID NO:111 from amino add 1 to amino acid 12. In further preferred embodiments, the present invention provides a polynucleotide encoding a protein comprising a fragment of the amino acid sequence of SEQ ID NO:111 having biological activity, the fragment preferably comprising eight (more preferably twenty, most preferably thirty) contiguous amino acids of SEQ ID NO:111, or a polynucleotide encoding a protein comprising a fragment of the amino acid sequence of SEQ ID NO:111 having biological activity, the fragment comprising the amino add sequence from amino acid 23 to amino acid 32 of SEQ ID NO:111.

Other embodiments provide the gene corresponding to the cDNA sequence of SEQ ID NO:110.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:110, but excluding the poly(A) tail at the 3′ end of SEQ ID NO:110; and
      (ab) the nucleotide sequence of the cDNA insert of clone AJ143_{—}1 deposited with the ATCC under accession number 98101;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:110, but excluding the poly(A) tail at the 3′ end of SEQ ID NO:110; and
      (bb) the nucleotide sequence of the cDNA insert of clone AJ143_{—}1 deposited with the ATCC under accession number 98101;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:110, and extending contiguously from a nucleotide sequence corresponding to the 5′ end of SEQ ID NO:110 to a nucleotide sequence corresponding to the 3′ end of SEQ ID NO:110, but excluding the poly(A) tail at the 3′ end of SEQ ID NO:110. Also preferably the polynucleotide isolated according to the above process comprises a nucleotide sequence corresponding to the cDNA sequence of SEQ ID NO:110 from nucleotide 827 to nucleotide 994, and extending contiguously from a nucleotide sequence corresponding to the 5′ end of said sequence of SEQ ID NO:110 from nucleotide 827 to nucleotide 994, to a nucleotide sequence corresponding to the 3′ end of said sequence of SEQ ID NO:110 from nucleotide 827 to nucleotide 994. Also preferably the polynucleotide isolated according to the above process comprises a nucleotide sequence corresponding to the cDNA sequence of SEQ ID NO:110 from nucleotide 869 to nucleotide 994, and extending contiguously from a nucleotide sequence corresponding to the 5 end of said sequence of SEQ ID NO:110 from nucleotide 869 to nucleotide 994, to a nucleotide sequence corresponding to the 3′ end of said sequence of SEQ ID NO:110 from nucleotide 869 to nucleotide 994. Also preferably the polynucleotide isolated according to the above process comprises a nucleotide sequence corresponding to the cDNA sequence of SEQ ID NO:110 from nucleotide 495 to nucleotide 665, and extending contiguously from a nucleotide sequence corresponding to the 5′ end of said sequence of SEQ ID NO:110 from nucleotide 495 to nucleotide 665, to a nucleotide sequence corresponding to the 3′ end of said sequence of SEQ ID NO:110 from nucleotide 495 to nucleotide 665.

In other embodiments, the present invention provides a composition comprising a protein, wherein said protein comprises an amino acid sequence selected from the group consisting of:
(a) the amino acid sequence of SEQ ID NO:111;
(b) the amino acid sequence of SEQ ID NO:111 from amino acid 1 to amino acid 12;
(c) a fragment of the amino acid sequence of SEQ ID NO:111, the fragment comprising eight contiguous amino acids of SEQ ID NO:111; and
(d) the amino acid sequence encoded by the cDNA insert of clone AJ143_{—}1 deposited with the ATCC under accession number 98101;
   the protein being substantially free from other mammalian proteins. Preferably such protein comprises the amino acid sequence of SEQ ID NO:111 or the amino acid sequence of SEQ ID NO:111 from amino acid 1 to amino acid 12. In further preferred embodiments, the present invention provides a protein comprising a fragment of the amino acid sequence of SEQ ID NO:111 having biological activity, the fragment preferably comprising eight (more preferably twenty, most preferably thirty) contiguous amino acids of SEQ ID NO:111, or a protein comprising a fragment of the amino acid sequence of SEQ ID NO:111 having biological activity, the fragment comprising the amino acid sequence from amino acid 23 to amino acid 32 of SEQ ID NO:111.

In one embodiment, the present invention provides a composition comprising an isolated polynucleotide selected from the group consisting of:
(a) a polynucleotide comprising the nucleotide sequence of SEQ ID NO:112;
(b) a polynucleotide comprising the nucleotide sequence of SEQ ID NO:112 from nucleotide 91 to nucleotide 204;
(c) a polynucleotide comprising the nucleotide sequence of SEQ ID NO:112 from nucleotide 66 to nucleotide 436;
(d) a polynucleotide comprising the nucleotide sequence of the full-length protein coding sequence of clone AJ168_{—}4 deposited with the ATCC under accession number 98101;
(e) a polynucleotide encoding the full-length protein encoded by the cDNA insert of clone AJ168_{—}4 deposited with the ATCC under accession number 98101;
(f) a polynucleotide comprising the nucleotide sequence of a mature protein coding sequence of clone AJ168_{—}4 deposited with the ATCC under accession number 98101;
(g) a polynucleotide encoding a mature protein encoded by the cDNA insert of clone AJ168_{—}4 deposited with the ATCC under accession number 98101;
(h) a polynucleotide encoding a protein comprising the amino acid sequence of SEQ ID NO:113;
(i) a polynucleotide encoding a protein comprising a fragment of the amino acid sequence of SEQ ID NO:113 having biological activity, the fragment comprising eight contiguous amino acids of SEQ ID NO:113;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:112.

Preferably, such polynucleotide comprises the nucleotide sequence of SEQ ID NO:112 from nucleotide 91 to nucleotide 204; the nucleotide sequence of SEQ ID NO:112 from nucleotide 66 to nucleotide 436; the nucleotide sequence of the full-length protein coding sequence of clone AJ168_{—}4 deposited with the ATCC under accession number 98101; or the nucleotide sequence of a mature protein coding sequence of clone AJ168_{—}4 deposited with the ATCC under accession number 98101. In other preferred embodiments, the polynucleotide encodes the full-length or a mature protein encoded by the cDNA insert of clone AJ168_{—}4 deposited with the ATCC under accession number 98101. In further preferred embodiments, the present invention provides a polynucleotide encoding a protein comprising a fragment of the amino acid sequence of SEQ ID NO:113 having biological activity, the fragment preferably comprising eight (more preferably twenty, most preferably thirty) contiguous amino acids of SEQ ID NO:113, or a polynucleotide encoding a protein comprising a fragment of the amino acid sequence of SEQ ID NO:113 having biological activity, the fragment comprising the amino acid sequence from amino acid 14 to amino acid 23 of SEQ ID NO:113.

Other embodiments provide the gene corresponding to the cDNA sequence of SEQ ID NO:112.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:112, but excluding the poly(A) tail at the 3′ end of SEQ ID NO:112; and
      (ab) the nucleotide sequence of the cDNA insert of clone AJ168_{—}4 deposited with the ATCC under accession number 98101;
   (ii) hybridizing said probe(s) to human genomic DNA in conditions at least as stringent as 4×SSC at 50 degrees C.; and
(iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:112, but excluding the poly(A) tail at the 3′ end of SEQ ID NO:112; and
      (bb) the nucleotide sequence of the cDNA insert of clone AJ168_{—}4 deposited with the ATCC under accession number 98101;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:112, and extending contiguously from a nucleotide sequence corresponding to the 5′ end of SEQ ID NO:112 to a nucleotide sequence corresponding to the 3′ end of SEQ ID NO:112, but excluding the poly(A) tail at the 3′ end of SEQ ID NO:112. Also preferably the polynucleotide isolated according to the above process comprises a nucleotide sequence corresponding to the cDNA sequence of SEQ ID NO:112 from nucleotide 91 to nucleotide 204, and extending contiguously from a nucleotide sequence corresponding to the 5′ end of said sequence of SEQ ID NO:112 from nucleotide 91 to nucleotide 204, to a nucleotide sequence corresponding to the 3′ end of said sequence of SEQ ID NO:112 from nucleotide 91 to nucleotide 204. Also preferably the polynucleotide isolated according to the above process comprises a nucleotide sequence corresponding to the cDNA sequence of SEQ ID NO:112 from nucleotide 66 to nucleotide 436, and extending contiguously from a nucleotide sequence corresponding to the 5′ end of said sequence of SEQ ID NO:112 from nucleotide 66 to nucleotide 436, to a nucleotide sequence corresponding to the 3′ end of said sequence of SEQ ID NO:112 from nucleotide 66 to nucleotide 436.

In other embodiments, the present invention provides a composition comprising a protein, wherein said protein comprises an amino acid sequence selected from the group consisting of:
(a) the amino acid sequence of SEQ ID NO:113;
(b) a fragment of the amino acid sequence of SEQ ID NO:113, the fragment comprising eight contiguous amino acids of SEQ ID NO:113; and
(c) the amino acid sequence encoded by the cDNA insert of clone AJ168_{—}4 deposited with the ATCC under accession number 98101;
   the protein being substantially free from other mammalian proteins. Preferably such protein comprises the amino acid sequence of SEQ ID NO:113. In further preferred embodiments, the present invention provides a protein comprising a fragment of the amino acid sequence of SEQ ID NO:113 having biological activity, the fragment preferably comprising eight (more preferably twenty, most preferably thirty) contiguous amino acids of SEQ ID NO:113, or a protein comprising a fragment of the amino acid sequence of SEQ ID NO:113 having biological activity, the fragment comprising the amino acid sequence from amino acid 14 to amino acid 23 of SEQ ID NO:113.

In one embodiment, the present invention provides a composition comprising an isolated polynucleotide selected from the group consisting of:
(a) a polynucleotide comprising the nucleotide sequence of SEQ ID NO:114;
(b) a polynucleotide comprising the nucleotide sequence of SEQ ID NO:114 from nucleotide 60 to nucleotide 230;
(c) a polynucleotide comprising the nucleotide sequence of SEQ ID NO:114 from nucleotide 84 to nucleotide 195;
(d) a polynucleotide comprising the nucleotide sequence of the full-length protein coding sequence of clone AK684_{—}1 deposited with the ATCC under accession number 98101;
(e) a polynucleotide encoding the full-length protein encoded by the cDNA insert of clone AK684_{—}1 deposited with the ATCC under accession number 98101;
(f) a polynucleotide comprising the nucleotide sequence of a mature protein coding sequence of clone AK684_{—}1 deposited with the ATCC under accession number 98101;
(g) a polynucleotide encoding a mature protein encoded by the cDNA insert of clone AK684_{—}1 deposited with the ATCC under accession number 98101;
(h) a polynucleotide encoding a protein comprising the amino acid sequence of SEQ ID NO:115;
(i) a polynucleotide encoding a protein comprising a fragment of the amino acid sequence of SEQ ID NO:115 having biological activity, the fragment comprising eight contiguous amino acids of SEQ ID NO:115;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:114.

Preferably, such polynucleotide comprises the nucleotide sequence of SEQ ID NO:114 from nucleotide 60 to nucleotide 230; the nucleotide sequence of SEQ ID NO:114 from nucleotide 84 to nucleotide 195; the nucleotide sequence of the full-length protein coding sequence of clone AK684_{—}1 deposited with the ATCC under accession number 98101; or the nucleotide sequence of a mature protein coding sequence of clone AK684_{—}1 deposited with the ATCC under accession number 98101. In other preferred embodiments, the polynucleotide encodes the full-length or a mature protein encoded by the cDNA insert of clone AK684_{—}1 deposited with the ATCC under accession number 98101. In further preferred embodiments, the present invention provides a polynucleotide encoding a protein comprising a fragment of the amino acid sequence of SEQ ID NO:115 having biological activity, the fragment preferably comprising eight (more preferably twenty, most preferably thirty) contiguous amino acids of SEQ ID NO:115, or a polynucleotide encoding a protein comprising a fragment of the amino acid sequence of SEQ ID NO:115 having biological activity, the fragment comprising the amino acid sequence from amino acid 23 to amino acid 32 of SEQ ID NO:115.

Other embodiments provide the gene corresponding to the cDNA sequence of SEQ ID NO:114.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:114, but excluding the poly(A) tail at the 3′ end of SEQ ID NO:114; and
      (ab) the nucleotide sequence of the cDNA insert of clone AK684_{—}1 deposited with the ATCC under accession number 98101;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:114, but excluding the poly(A) tail at the 3′ end of SEQ ID NO:114; and
      (bb) the nucleotide sequence of the cDNA insert of clone AK684_{—}1 deposited with the ATCC under accession number 98101;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:114, and extending contiguously from a nucleotide sequence corresponding to the 5′ end of SEQ ID NO:114 to a nucleotide sequence corresponding to the 3′ end of SEQ ID NO:114, but excluding the poly(A) tail at the 3′ end of SEQ ID NO:114. Also preferably the polynucleotide isolated according to the above process comprises a nucleotide sequence corresponding to the cDNA sequence of SEQ ID NO:114 from nucleotide 60 to nucleotide 230, and extending contiguously from a nucleotide sequence corresponding to the 5′ end of said sequence of SEQ ID NO:114 from nucleotide 60 to nucleotide 230, to a nucleotide sequence corresponding to the 3′ end of said sequence of SEQ ID NO:114 from nucleotide 60 to nucleotide 230. Also preferably the polynucleotide isolated according to the above process comprises a nucleotide sequence corresponding to the cDNA sequence of SEQ ID NO:114 from nucleotide 84 to nucleotide 195, and extending contiguously from a nucleotide sequence corresponding to the 5′ end of said sequence of SEQ ID NO:114 from nucleotide 84 to nucleotide 195, to a nucleotide sequence corresponding to the 3′ end of said sequence of SEQ ID NO:114 from nucleotide 84 to nucleotide 195.

In other embodiments, the present invention provides a composition comprising a protein, wherein said protein comprises an amino acid sequence selected from the group consisting of:
(a) the amino acid sequence of SEQ ID NO:115;
(b) a fragment of the amino acid sequence of SEQ ID NO:115, the fragment comprising eight contiguous amino acids of SEQ ID NO:115; and
(c) the amino acid sequence encoded by the cDNA insert of clone AK684_{—}1 deposited with the ATCC under accession number 98101;
   the protein being substantially free from other mammalian proteins. Preferably such protein comprises the amino acid sequence of SEQ ID NO:115. In further preferred embodiments, the present invention provides a protein comprising a fragment of the amino acid sequence of SEQ ID NO:115 having biological activity, the fragment preferably comprising eight (more preferably twenty, most preferably thirty) contiguous amino acids of SEQ ID NO:115, or a protein comprising a fragment of the amino acid sequence of SEQ ID NO:115 having biological activity, the fragment comprising the amino acid sequence from amino acid 23 to amino acid 32 of SEQ ID NO:115.

In one embodiment, the present invention provides a composition comprising an isolated polynucleotide selected from the group consisting of:
(a) a polynucleotide comprising the nucleotide sequence of SEQ ID NO:116;
(b) a polynucleotide comprising the nucleotide sequence of SEQ ID NO:116 from nucleotide 812 to nucleotide 2731;
(c) a polynucleotide comprising the nucleotide sequence of SEQ ID NO:116 from nucleotide 944 to nucleotide 2731;
(d) a polynucleotide comprising the nucleotide sequence of SEQ ID NO:116 from nucleotide 959 to nucleotide 1186;
(e) a polynucleotide comprising the nucleotide sequence of the full-length protein coding sequence of clone AS209_{—}1 deposited with the ATCC under accession number 98101;
(f) a polynucleotide encoding the full-length protein encoded by the cDNA insert of clone AS209_{—}1 deposited with the ATCC under accession number 98101;
(g) a polynucleotide comprising the nucleotide sequence of a mature protein coding sequence of clone AS209_{—}1 deposited with the ATCC under accession number 98101;
(h) a polynucleotide encoding a mature protein encoded by the cDNA insert of clone AS209_{—}1 deposited with the ATCC under accession number 98101;
(i) a polynucleotide encoding a protein comprising the amino acid sequence of SEQ ID NO:117;
(j) a polynucleotide encoding a protein comprising a fragment of the amino acid sequence of SEQ ID NO:117 having biological activity, the fragment comprising eight contiguous amino acids of SEQ ID NO:117;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:116.

Preferably, such polynucleotide comprises the nucleotide sequence of SEQ ID NO:116 from nucleotide 812 to nucleotide 2731; the nucleotide sequence of SEQ ID NO:116 from nucleotide 944 to nucleotide 2731; the nucleotide sequence of SEQ ID NO:116 from nucleotide 959 to nucleotide 1186; the nucleotide sequence of the full-length protein coding sequence of clone AS209_{—}1 deposited with the ATCC under accession number 98101; or the nucleotide sequence of a mature protein coding sequence of clone AS209_{—}1 deposited with the ATCC under accession number 98101. In other preferred embodiments, the polynucleotide encodes the full-length or a mature protein encoded by the cDNA insert of clone AS209_{—}1 deposited with the ATCC under accession number 98101. In yet other preferred embodiments, the present invention provides a polynucleotide encoding a protein comprising the amino acid sequence of SEQ ID NO:117 from amino acid 50 to amino acid 125. In further preferred embodiments, the present invention provides a polynucleotide encoding a protein comprising a fragment of the amino acid sequence of SEQ ID NO:117 having biological activity, the fragment preferably comprising eight (more preferably twenty, most preferably thirty) contiguous amino acids of SEQ ID NO:117, or a polynucleotide encoding a protein comprising a fragment of the amino acid sequence of SEQ ID NO:117 having biological activity, the fragment comprising the amino acid sequence from amino acid 315 to amino acid 324 of SEQ ID NO:117.

Other embodiments provide the gene corresponding to the cDNA sequence of SEQ ID NO:116.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:116, but excluding the poly(A) tail at the 3′ end of SEQ ID NO:116; and
      (ab) the nucleotide sequence of the cDNA insert of clone AS209_{—}1 deposited with the ATCC under accession number 98101;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:116, but excluding the poly(A) tail at the 3′ end of SEQ ID NO:116; and
      (bb) the nucleotide sequence of the cDNA insert of clone AS209_{—}1 deposited with the ATCC under accession number 98101;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:116, and extending contiguously from a nucleotide sequence corresponding to the 5′ end of SEQ ID NO:116 to a nucleotide sequence corresponding to the 3′ end of SEQ ID NO:116, but excluding the poly(A) tail at the 3′ end of SEQ ID NO:116. Also preferably the polynucleotide isolated according to the above process comprises a nucleotide sequence corresponding to the cDNA sequence of SEQ ID NO:116 from nucleotide 812 to nucleotide 2731, and extending contiguously from a nucleotide sequence corresponding to the 5′ end of said sequence of SEQ ID NO:116 from nucleotide 812 to nucleotide 2731, to a nucleotide sequence corresponding to the 3′ end of said sequence of SEQ ID NO:116 from nucleotide 812 to nucleotide 2731. Also preferably the polynucleotide isolated according to the above process comprises a nucleotide sequence corresponding to the cDNA sequence of SEQ ID NO:116 from nucleotide 944 to nucleotide 2731, and extending contiguously from a nucleotide sequence corresponding to the 5′ end of said sequence of SEQ ID NO:116 from nucleotide 944 to nucleotide 2731, to a nucleotide sequence corresponding to the 3′ end of said sequence of SEQ ID NO:116 from nucleotide 944 to nucleotide 2731. Also preferably the polynucleotide isolated according to the above process comprises a nucleotide sequence corresponding to the cDNA sequence of SEQ ID NO:116 from nucleotide 959 to nucleotide 1186, and extending contiguously from a nucleotide sequence corresponding to the 5′ end of said sequence of SEQ ID NO:116 from nucleotide 959 to nucleotide 1186, to a nucleotide sequence corresponding to the 3′ end of said sequence of SEQ ID NO:116 from nucleotide 959 to nucleotide 1186.

In other embodiments, the present invention provides a composition comprising a protein, wherein said protein comprises an amino acid sequence selected from the group consisting of:
(a) the amino acid sequence of SEQ ID NO:117;
(b) the amino acid sequence of SEQ ID NO:117 from amino acid 50 to amino acid 125;
(c) a fragment of the amino acid sequence of SEQ ID NO:117, the fragment comprising eight contiguous amino acids of SEQ ID NO:117; and
(d) the amino acid sequence encoded by the cDNA insert of clone AS209_{—}1 deposited with the ATCC under accession number 98101; the protein being substantially free from other mammalian proteins. Preferably such protein comprises the amino acid sequence of SEQ ID NO:117 or the amino acid sequence of SEQ ID NO:117 from amino acid 50 to amino acid 125. In further preferred embodiments, the present invention provides a protein comprising a fragment of the amino acid sequence of SEQ ID NO:117 having biological activity, the fragment preferably comprising eight (more preferably twenty, most preferably thirty) contiguous amino acids of SEQ ID NO:117, or a protein comprising a fragment of the amino acid sequence of SEQ ID NO:117 having biological activity, the fragment comprising the amino acid sequence from amino acid 315 to amino acid 324 of SEQ ID NO:117.

In one embodiment, the present invention provides a composition comprising an isolated polynucleotide selected from the group consisting of:
(a) a polynucleotide comprising the nucleotide sequence of SEQ ID NO:118;
(b) a polynucleotide comprising the nucleotide sequence of SEQ ID NO:118 from nucleotide 2196 to nucleotide 2708;
(c) a polynucleotide comprising the nucleotide sequence of SEQ ID NO:118 from nucleotide 622 to nucleotide 890;
(d) a polynucleotide comprising the nucleotide sequence of the full-length protein coding sequence of clone AX56_{—}28 deposited with the ATCC under accession number 98180;
(e) a polynucleotide encoding the full-length protein encoded by the cDNA insert of clone AX56_{—}28 deposited with the ATCC under accession number 98180;
(f) a polynucleotide comprising the nucleotide sequence of a mature protein coding sequence of clone AX56_{—}28 deposited with the ATCC under accession number 98180;
(g) a polynucleotide encoding a mature protein encoded by the cDNA insert of clone AX56_{—}28 deposited with the ATCC under accession number 98180;
(h) a polynucleotide encoding a protein comprising the amino acid sequence of SEQ ID NO:119;
(i) a polynucleotide encoding a protein comprising a fragment of the amino acid sequence of SEQ ID NO:119 having biological activity, the fragment comprising eight contiguous amino acids of SEQ ID NO:119;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:118.

Preferably, such polynucleotide comprises the nucleotide sequence of SEQ ID NO:118 from nucleotide 2196 to nucleotide 2708; the nucleotide sequence of SEQ ID NO:118 from nucleotide 622 to nucleotide 890; the nucleotide sequence of the full-length protein coding sequence of clone AX56_{—}28 deposited with the ATCC under accession number 98180; or the nucleotide sequence of a mature protein coding sequence of clone AX56_{—}28 deposited with the ATCC under accession number 98180. In other preferred embodiments, the polynucleotide encodes the full-length or a mature protein encoded by the cDNA insert of clone AX56_{—}28 deposited with the ATCC under accession number 98180. In further preferred embodiments, the present invention provides a polynucleotide encoding a protein comprising a fragment of the amino acid sequence of SEQ ID NO:119 having biological activity, the fragment preferably comprising eight (more preferably twenty, most preferably thirty) contiguous amino acids of SEQ ID NO:119, or a polynucleotide encoding a protein comprising a fragment of the amino acid sequence of SEQ ID NO:119 having biological activity, the fragment comprising the amino acid sequence from amino acid 80 to amino acid 89 of SEQ ID NO:119.

Other embodiments provide the gene corresponding to the cDNA sequence of SEQ ID NO:118.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:118, but excluding the poly(A) tail at the 3′ end of SEQ ID NO:118; and
      (ab) the nucleotide sequence of the cDNA insert of clone AX56_{—}28 deposited with the ATCC under accession number 98180;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:118, but excluding the poly(A) tail at the 3′ end of SEQ ID NO:118; and
      (bb) the nucleotide sequence of the cDNA insert of clone AX56_{—}28 deposited with the ATCC under accession number 98180;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:118, and extending contiguously from a nucleotide sequence corresponding to the 5′ end of SEQ ID NO:118 to a nucleotide sequence corresponding to the 3′ end of SEQ ID NO:118, but excluding the poly(A) tail at the 3′ end of SEQ ID NO:118. Also preferably the polynucleotide isolated according to the above process comprises a nucleotide sequence corresponding to the cDNA sequence of SEQ ID NO:118 from nucleotide 2196 to nucleotide 2708, and extending contiguously from a nucleotide sequence corresponding to the 5′ end of said sequence of SEQ ID NO:118 from nucleotide 2196 to nucleotide 2708, to a nucleotide sequence corresponding to the 3′ end of said sequence of SEQ ID NO:118 from nucleotide 2196 to nucleotide 2708. Also preferably the polynucleotide isolated according to the above process comprises a nucleotide sequence corresponding to the cDNA sequence of SEQ ID NO:118 from nucleotide 622 to nucleotide 890, and extending contiguously from a nucleotide sequence corresponding to the 5′ end of said sequence of SEQ ID NO:118 from nucleotide 622 to nucleotide 890, to a nucleotide sequence corresponding to the 3′ end of said sequence of SEQ ID NO:118 from nucleotide 622 to nucleotide 890.

In other embodiments, the present invention provides a composition comprising a protein, wherein said protein comprises an amino acid sequence selected from the group consisting of:
(a) the amino acid sequence of SEQ ID NO:119;
(b) a fragment of the amino acid sequence of SEQ ID NO:119, the fragment comprising eight contiguous amino acids of SEQ ID NO:119; and
(c) the amino acid sequence encoded by the cDNA insert of clone AX56_{—}28 deposited with the ATCC under accession number 98180;
   the protein being substantially free from other mammalian proteins. Preferably such protein comprises the amino acid sequence of SEQ ID NO:119. In further preferred embodiments, the present invention provides a protein comprising a fragment of the amino acid sequence of SEQ ID NO:119 having biological activity, the fragment preferably comprising eight (more preferably twenty, most preferably thirty) contiguous amino acids of SEQ ID NO:119, or a protein comprising a fragment of the amino acid sequence of SEQ ID NO:119 having biological activity, the fragment comprising the amino acid sequence from amino acid 80 to amino acid 89 of SEQ ID NO:119.

In one embodiment, the present invention provides a composition comprising an isolated polynucleotide selected from the group consisting of:
(a) a polynucleotide comprising the nucleotide sequence of SEQ ID NO:120;
(b) a polynucleotide comprising the nucleotide sequence of SEQ. ID NO:120 from nucleotide 51 to nucleotide 1319;
(c) a polynucleotide comprising the nucleotide sequence of SEQ ID NO:120 from nucleotide 126 to nucleotide 1319;
(d) a polynucleotide comprising the nucleotide sequence of SEQ ID NO:120 from nucleotide 409 to nucleotide 495;
(e) a polynucleotide comprising the nucleotide sequence of the full-length protein coding sequence of clone AX92_{—}3 deposited with the ATCC under accession number 98101;
(f) a polynucleotide encoding the full-length protein encoded by the cDNA insert of clone AX92_{—}3 deposited with the ATCC under accession number 98101;
(g) a polynucleotide comprising the nucleotide sequence of a mature protein coding sequence of clone AX92_{—}3 deposited with the ATCC under accession number 98101;
(h) a polynucleotide encoding a mature protein encoded by the cDNA insert of clone AX92_{—}3 deposited with the ATCC under accession number 98101;
(i) a polynucleotide encoding a protein comprising the amino acid sequence of SEQ ID NO:121;
(j) a polynucleotide encoding a protein comprising a fragment of the amino acid sequence of SEQ ID NO:121 having biological activity, the fragment comprising eight contiguous amino acids of SEQ ID NO:121;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:120.

Preferably, such polynucleotide comprises the nucleotide sequence of SEQ ID NO:120 from nucleotide 51 to nucleotide 1319; the nucleotide sequence of SEQ ID NO:120 from nucleotide 126 to nucleotide 1319; the nucleotide sequence of SEQ ID NO:120 from nucleotide 409 to nucleotide 495; the nucleotide sequence of the full-length protein coding sequence of clone AX92_{—}3 deposited with the ATCC under accession number 98101; or the nucleotide sequence of a mature protein coding sequence of clone AX92_{—}3 deposited with the ATCC under accession number 98101. In other preferred embodiments, the polynucleotide encodes the full-length or a mature protein encoded by the cDNA insert of clone AX92_{—}3 deposited with the ATCC under accession number 98101. In further preferred embodiments, the present invention provides a polynucleotide encoding a protein comprising a fragment of the amino acid sequence of SEQ ID NO:121 having biological activity, the fragment preferably comprising eight (more preferably twenty, most preferably thirty) contiguous amino acids of SEQ ID NO:121, or a polynucleotide encoding a protein comprising a fragment of the amino acid sequence of SEQ ID NO:121 having biological activity, the fragment comprising the amino acid sequence from amino acid 206 to amino acid 215 of SEQ ID NO:121.

Other embodiments provide the gene corresponding to the cDNA sequence of SEQ ID NO:120.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:120, but excluding the poly(A) tail at the 3′ end of SEQ ID NO:120; and
      (ab) the nucleotide sequence of the cDNA insert of clone AX92_{—}3 deposited with the ATCC under accession number 98101;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:120, but excluding the poly(A) tail at the 3′ end of SEQ ID NO:120; and
      (bb) the nucleotide sequence of the cDNA insert of clone AX92_{—}3 deposited with the ATCC under accession number 98101;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:120, and extending contiguously from a nucleotide sequence corresponding to the 5′ end of SEQ ID NO:120 to a nucleotide sequence corresponding to the 3′ end of SEQ ID NO:120, but excluding the poly(A) tail at the 3′ end of SEQ ID NO:120. Also preferably the polynucleotide isolated according to the above process comprises a nucleotide sequence corresponding to the cDNA sequence of SEQ ID NO:120 from nucleotide 51 to nucleotide 1319, and extending contiguously from a nucleotide sequence corresponding to the 5′ end of said sequence of SEQ ID NO:120 from nucleotide 51 to nucleotide 1319, to a nucleotide sequence corresponding to the 3′ end of said sequence of SEQ ID NO:120 from nucleotide 51 to nucleotide 1319. Also preferably the polynucleotide isolated according to the above process comprises a nucleotide sequence corresponding to the cDNA sequence of SEQ ID NO:120 from nucleotide 126 to nucleotide 1319, and extending contiguously from a nucleotide sequence corresponding to the 5′ end of said sequence of SEQ ID NO:120 from nucleotide 126 to nucleotide 1319, to a nucleotide sequence corresponding to the 3′ end of said sequence of SEQ ID NO:120 from nucleotide 126 to nucleotide 1319. Also preferably the polynucleotide isolated according to the above process comprises a nucleotide sequence corresponding to the cDNA sequence of SEQ ID NO:120 from nucleotide 409 to nucleotide 495, and extending contiguously from a nucleotide sequence corresponding to the 5′ end of said sequence of SEQ ID NO:120 from nucleotide 409 to nucleotide 495, to a nucleotide sequence corresponding to the 3′ end of said sequence of SEQ ID NO:120 from nucleotide 409 to nucleotide 495.

In other embodiments, the present invention provides a composition comprising a protein, wherein said protein comprises an amino acid sequence selected from the group consisting of:
(a) the amino acid sequence of SEQ ID NO:121;
(b) a fragment of the amino acid sequence of SEQ ID NO:121, the fragment comprising eight contiguous amino acids of SEQ ID NO:121; and
(c) the amino acid sequence encoded by the cDNA insert of clone AX92_{—}3 deposited with the ATCC under accession number 98101;
   the protein being substantially free from other mammalian proteins. Preferably such protein comprises the amino acid sequence of SEQ ID NO:121. In further preferred embodiments, the present invention provides a protein comprising a fragment of the amino acid sequence of SEQ ID NO:121 having biological activity, the fragment preferably comprising eight (more preferably twenty, most preferably thirty) contiguous amino adds of SEQ ID NO:121, or a protein comprising a fragment of the amino acid sequence of SEQ ID NO:121 having biological activity, the fragment comprising the amino acid sequence from amino acid 206 to amino acid 215 of SEQ ID NO:121.

In one embodiment, the present invention provides a composition comprising an isolated polynucleotide selected from the group consisting of:
(a) a polynucleotide comprising the nucleotide sequence of SEQ ID NO:122;
(b) a polynucleotide comprising the nucleotide sequence of SEQ ID NO:122 from nucleotide 896 to nucleotide 1294;
(c) a polynucleotide comprising the nucleotide sequence of SEQ ID NO:122 from nucleotide 1253 to nucleotide 1294;
(d) a polynucleotide comprising the nucleotide sequence of SEQ ID NO:122 from nucleotide 1262 to nucleotide 1402;
(e) a polynucleotide comprising the nucleotide sequence of the full-length protein coding sequence of clone BF245_{—}1 deposited with the ATCC under accession number 98101;
(f) a polynucleotide encoding the full-length protein encoded by the cDNA insert of clone BF245_{—}1 deposited with the ATCC under accession number 98101;
(g) a polynucleotide comprising the nucleotide sequence of a mature protein coding sequence of clone BF245_{—}1 deposited with the ATCC under accession number 98101;
(h) a polynucleotide encoding a mature protein encoded by the cDNA insert of clone BF245_{—}1 deposited with the ATCC under accession number 98101;
(i) a polynucleotide encoding a protein comprising the amino acid sequence of SEQ ID NO:123;
(j) a polynucleotide encoding a protein comprising a fragment of the amino acid sequence of SEQ ID NO:123 having biological activity, the fragment comprising eight contiguous amino acids of SEQ ID NO:123;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:122.

Preferably, such polynucleotide comprises the nucleotide sequence of SEQ ID NO:122 from nucleotide 896 to nucleotide 1294; the nucleotide sequence of SEQ ID NO:122 from nucleotide 1253 to nucleotide 1294; the nucleotide sequence of SEQ ID NO:122 from nucleotide 1262 to nucleotide 1402; the nucleotide sequence of the full-length protein coding sequence of clone BF245_{—}1 deposited with the ATCC under accession number 98101; or the nucleotide sequence of a mature protein coding sequence of clone BF245_{—}1 deposited with the ATCC under accession number 98101. In other preferred embodiments, the polynucleotide encodes the full-length or a mature protein encoded by the cDNA insert of clone BF245_{—}1 deposited with the ATCC under accession number 98101. In yet other preferred embodiments, the present invention provides a polynucleotide encoding a protein comprising the amino acid sequence of SEQ ID NO:123 from amino acid 1 to amino acid 11. In further preferred embodiments, the present invention provides a polynucleotide encoding a protein comprising a fragment of the amino acid sequence of SEQ ID NO:123 having biological activity, the fragment preferably comprising eight (more preferably twenty, most preferably thirty) contiguous amino acids of SEQ ID NO:123, or a polynucleotide encoding a protein comprising a fragment of the amino acid sequence of SEQ ID NO:123 having biological activity, the fragment comprising the amino acid sequence from amino acid 61 to amino acid 70 of SEQ ID NO:123.

Other embodiments provide the gene corresponding to the cDNA sequence of SEQ ID NO:122.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:122, but excluding the poly(A) tail at the 3′ end of SEQ ID NO:122; and
      (ab) the nucleotide sequence of the cDNA insert of clone BF245_{—}1 deposited with the ATCC under accession number 98101;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:122, but excluding the poly(A) tail at the 3′ end of SEQ ID NO:122; and
      (bb) the nucleotide sequence of the cDNA insert of clone BF245_{—}1 deposited with the ATCC under accession number 98101;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:122, and extending contiguously from a nucleotide sequence corresponding to the 5′ end of SEQ ID NO:122 to a nucleotide sequence corresponding to the 3′ end of SEQ ID NO:122, but excluding the poly(A) tail at the 3′ end of SEQ ID NO:122. Also preferably the polynucleotide isolated according to the above process comprises a nucleotide sequence corresponding to the cDNA sequence of SEQ ID NO:122 from nucleotide 896 to nucleotide 1294, and extending contiguously from a nucleotide sequence corresponding to the 5′ end of said sequence of SEQ ID NO:122 from nucleotide 896 to nucleotide 1294, to a nucleotide sequence corresponding to the 3′ end of said sequence of SEQ ID NO:122 from nucleotide 896 to nucleotide 1294. Also preferably the polynucleotide isolated according to the above process comprises a nucleotide sequence corresponding to the cDNA sequence of SEQ ID NO:122 from nucleotide 1253 to nucleotide 1294, and extending contiguously from a nucleotide sequence corresponding to the 5′ end of said sequence of SEQ ID NO:122 from nucleotide 1253 to nucleotide 1294, to a nucleotide sequence corresponding to the 3′ end of said sequence of SEQ ID NO:122 from nucleotide 1253 to nucleotide 1294. Also preferably the polynucleotide isolated according to the above process comprises a nucleotide sequence corresponding to the cDNA sequence of SEQ ID NO:122 from nucleotide 1262 to nucleotide 1402, and extending contiguously from a nucleotide sequence corresponding to the 5′ end of said sequence of SEQ ID NO:122 from nucleotide 1262 to nucleotide 1402, to a nucleotide sequence corresponding to the 3′ end of said sequence of SEQ ID NO:122 from nucleotide 1262 to nucleotide 1402.

In other embodiments, the present invention provides a composition comprising a protein, wherein said protein comprises an amino acid sequence selected from the group consisting of:
(a) the amino acid sequence of SEQ ID NO:123;
(b) the amino acid sequence of SEQ ID NO:123 from amino acid 1 to amino acid 11;
(c) a fragment of the amino acid sequence of SEQ ID NO:123, the fragment comprising eight contiguous amino acids of SEQ ID NO:123; and
(d) the amino acid sequence encoded by the cDNA insert of clone BF245_{—}1 deposited with the ATCC under accession number 98101;
   the protein being substantially free from other mammalian proteins. Preferably such protein comprises the amino acid sequence of SEQ ID NO:123 or the amino acid sequence of SEQ ID NO:123 from amino acid 1 to amino acid 11. In further preferred embodiments, the present invention provides a protein comprising a fragment of the amino acid sequence of SEQ ID NO:123 having biological activity, the fragment preferably comprising eight (more preferably twenty, most preferably thirty) contiguous amino acids of SEQ ID NO:123, or a protein comprising a fragment of the amino acid sequence of SEQ ID NO:123 having biological activity, the fragment comprising the amino acid sequence from amino acid 61 to amino acid 70 of SEQ ID NO:123.

In one embodiment, the present invention provides a composition comprising an isolated polynucleotide selected from the group consisting of:
(a) a polynucleotide comprising the nucleotide sequence of SEQ ID NO:124;
(b) a polynucleotide comprising the nucleotide sequence of SEQ ID NO:124 from nucleotide 322 to nucleotide 774;
(c) a polynucleotide comprising the nucleotide sequence of SEQ ID NO:124 from nucleotide 538 to nucleotide 641;
(d) a polynucleotide comprising the nucleotide sequence of the full-length protein coding sequence of clone BG33_{—}7 deposited with the ATCC under accession number 98101;
(e) a polynucleotide encoding the full-length protein encoded by the cDNA insert of clone BG33_{—}7 deposited with the ATCC under accession number 98101;
(f) a polynucleotide comprising the nucleotide sequence of a mature protein coding sequence of clone BG33_{—}7 deposited with the ATCC under accession number 98101;
(g) a polynucleotide encoding a mature protein encoded by the cDNA insert of clone BG33_{—}7 deposited with the ATCC under accession number 98101;
(h) a polynucleotide encoding a protein comprising the amino acid sequence of SEQ ID NO:125;
(i) a polynucleotide encoding a protein comprising a fragment of the amino acid sequence of SEQ ID NO:125 having biological activity, the fragment comprising eight contiguous amino acids of SEQ ID NO:125;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:124.

Preferably, such polynucleotide comprises the nucleotide sequence of SEQ ID NO:124 from nucleotide 322 to nucleotide 774; the nucleotide sequence of SEQ ID NO:124 from nucleotide 538 to nucleotide 641; the nucleotide sequence of the full-length protein coding sequence of clone BG33_{—}7 deposited with the ATCC under accession number 98101; or the nucleotide sequence of a mature protein coding sequence of clone BG33_{—}7 deposited with the ATCC under accession number 98101. In other preferred embodiments, the polynucleotide encodes the full-length or a mature protein encoded by the cDNA insert of clone BG33_{—}7 deposited with the ATCC under accession number 98101. In yet other preferred embodiments, the present invention provides a polynucleotide encoding a protein comprising the amino acid sequence of SEQ ID NO:125 from amino acid 72 to amino acid 106. In further preferred embodiments, the present invention provides a polynucleotide encoding a protein comprising a fragment of the amino acid sequence of SEQ ID NO:125 having biological activity, the fragment preferably comprising eight (more preferably twenty, most preferably thirty) contiguous amino acids of SEQ ID NO:125, or a polynucleotide encoding a protein comprising a fragment of the amino acid sequence of SEQ ID NO:125 having biological activity, the fragment comprising the amino acid sequence from amino acid 70 to amino acid 79 of SEQ ID NO:125.

Other embodiments provide the gene corresponding to the cDNA sequence of SEQ ID NO:124.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:124, but excluding the poly(A) tail at the 3′ end of SEQ ID NO:124; and
      (ab) the nucleotide sequence of the cDNA insert of clone BG33_{—}7 deposited with the ATCC under accession number 98101;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:124, but excluding the poly(A) tail at the 3′ end of SEQ ID NO:124; and
      (bb) the nucleotide sequence of the cDNA insert of clone BG33_{—}7 deposited with the ATCC under accession number 98101;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:124, and extending contiguously from a nucleotide sequence corresponding to the 5′ end of SEQ ID NO:124 to a nucleotide sequence corresponding to the 3′ end of SEQ ID NO:124, but excluding the poly(A) tail at the 3′ end of SEQ ID NO:124. Also preferably the polynucleotide isolated according to the above process comprises a nucleotide sequence corresponding to the cDNA sequence of SEQ ID NO:124 from nucleotide 322 to nucleotide 774, and extending contiguously from a nucleotide sequence corresponding to the 5′ end of said sequence of SEQ ID NO:124 from nucleotide 322 to nucleotide 774, to a nucleotide sequence corresponding to the 3′ end of said sequence of SEQ ID NO:124 from nucleotide 322 to nucleotide 774. Also preferably the polynucleotide isolated according to the above process comprises a nucleotide sequence corresponding to the cDNA sequence of SEQ ID NO:124 from nucleotide 538 to nucleotide 641, and extending contiguously from a nucleotide sequence corresponding to the 5′ end of said sequence of SEQ ID NO:124 from nucleotide 538 to nucleotide 641, to a nucleotide sequence corresponding to the 3′ end of said sequence of SEQ ID NO:124 from nucleotide 538 to nucleotide 641.

In other embodiments, the present invention provides a composition comprising a protein, wherein said protein comprises an amino acid sequence selected from the group consisting of:
(a) the amino acid sequence of SEQ ID NO:125;
(b) the amino acid sequence of SEQ ID NO:125 from amino acid 72 to amino acid 106;
(c) a fragment of the amino acid sequence of SEQ ID NO:125, the fragment comprising eight contiguous amino acids of SEQ ID NO:125; and
(d) the amino acid sequence encoded by the cDNA insert of clone BG33_{—}7 deposited with the ATCC under accession number 98101;
   the protein being substantially free from other mammalian proteins. Preferably such protein comprises the amino acid sequence of SEQ ID NO:125 or the amino acid sequence of SEQ ID NO:125 from amino acid 72 to amino acid 106. In further preferred embodiments, the present invention provides a protein comprising a fragment of the amino acid sequence of SEQ ID NO:125 having biological activity, the fragment preferably comprising eight (more preferably twenty, most preferably thirty) contiguous amino acids of SEQ ID NO:125, or a protein comprising a fragment of the amino acid sequence of SEQ ID NO:125 having biological activity, the fragment comprising the amino acid sequence from amino acid 70 to amino acid 79 of SEQ ID NO:125.

In one embodiment, the present invention provides a composition comprising an isolated polynucleotide selected from the group consisting of:
(a) a polynucleotide comprising the nucleotide sequence of SEQ ID NO:126;
(b) a polynucleotide comprising the nucleotide sequence of SEQ ID NO:126 from nucleotide 80 to nucleotide 2794;
(c) a polynucleotide comprising the nucleotide sequence of SEQ ID NO:126 from nucleotide 415 to nucleotide 1750;
(d) a polynucleotide comprising the nucleotide sequence of the full-length protein coding sequence of clone BM46_{—}10 deposited with the ATCC under accession number 98152;
(e) a polynucleotide encoding the full-length protein encoded by the cDNA insert of clone BM46_{—}10 deposited with the ATCC under accession number 98152;
(f) a polynucleotide comprising the nucleotide sequence of a mature protein coding sequence of clone BM46_{—}10 deposited with the ATCC under accession number 98152;
(g) a polynucleotide encoding a mature protein encoded by the cDNA insert of clone BM46_{—}10 deposited with the ATCC under accession number 98152;
(h) a polynucleotide encoding a protein comprising the amino acid sequence of SEQ ID NO:127;
(i) a polynucleotide encoding a protein comprising a fragment of the amino acid sequence of SEQ ID NO:127 having biological activity, the fragment comprising eight contiguous amino acids of SEQ ID NO:127;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:126.

Preferably, such polynucleotide comprises the nucleotide sequence of SEQ ID NO:126 from nucleotide 80 to nucleotide 2794; the nucleotide sequence of SEQ ID NO:126 from nucleotide 415 to nucleotide 1750; the nucleotide sequence of SEQ ID NO:126 from nucleotide 1 to nucleotide 338; the nucleotide sequence of SEQ ID NO:126 from nucleotide 1768 to nucleotide 3444; the nucleotide sequence of the full-length protein coding sequence of clone BM46_{—}10 deposited with the ATCC under accession number 98152; or the nucleotide sequence of a mature protein coding sequence of clone BM46_{—}10 deposited with the ATCC under accession number 98152. In other preferred embodiments, the polynucleotide encodes the full-length or a mature protein encoded by the cDNA insert of clone BM46_{—}10 deposited with the ATCC under accession number 98152. In yet other preferred embodiments, the present invention provides a polynucleotide encoding a protein comprising the amino acid sequence of SEQ ID NO:127 from amino acid 1 to amino acid 86; or a polynucleotide encoding a protein comprising the amino acid sequence of SEQ ID NO:127 from amino acid 113 to amino acid 557. In further preferred embodiments, the present invention provides a polynucleotide encoding a protein comprising a fragment of the amino acid sequence of SEQ ID NO:127 having biological activity, the fragment preferably comprising eight (more preferably twenty, most preferably thirty) contiguous amino acids of SEQ ID NO:127, or a polynucleotide encoding a protein comprising a fragment of the amino acid sequence of SEQ ID NO:127 having biological activity, the fragment comprising the amino acid sequence from amino acid 282 to amino acid 291 of SEQ ID NO:127.

Other embodiments provide the gene corresponding to the cDNA sequence of SEQ ID NO:126.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:126, but excluding the poly(A) tail at the 3′ end of SEQ ID NO:126; and
      (ab) the nucleotide sequence of the cDNA insert of clone BM46_{—}10 deposited with the ATCC under accession number 98152;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:126, but excluding the poly(A) tail at the 3′ end of SEQ ID NO:126; and
      (bb) the nucleotide sequence of the cDNA insert of clone BM46_{—}10 deposited with the ATCC under accession number 98152;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:126, and extending contiguously from a nucleotide sequence corresponding to the 5′ end of SEQ ID NO:126 to a nucleotide sequence corresponding to the 3′ end of SEQ ID NO:126, but excluding the poly(A) tail at the 3′ end of SEQ ID NO:126. Also preferably the polynucleotide isolated according to the above process comprises a nucleotide sequence corresponding to the cDNA sequence of SEQ ID NO:126 from nucleotide 80 to nucleotide 2794, and extending contiguously from a nucleotide sequence corresponding to the 5′ end of said sequence of SEQ ID NO:126 from nucleotide 80 to nucleotide 2794, to a nucleotide sequence corresponding to the 3′ end of said sequence of SEQ ID NO:126 from nucleotide 80 to nucleotide 2794. Also preferably the polynucleotide isolated according to the above process comprises a nucleotide sequence corresponding to the cDNA sequence of SEQ ID NO:126 from nucleotide 415 to nucleotide 1750, and extending contiguously from a nucleotide sequence corresponding to the 5′ end of said sequence of SEQ ID NO:126 from nucleotide 415 to nucleotide 1750, to a nucleotide sequence corresponding to the 3′ end of said sequence of SEQ ID NO:126 from nucleotide 415 to nucleotide 1750. Also preferably the polynucleotide isolated according to the above process comprises a nucleotide sequence corresponding to the cDNA sequence of SEQ ID NO:126 from nucleotide 1 to nucleotide 338, and extending contiguously from a nucleotide sequence corresponding to the 5′ end of said sequence of SEQ ID NO:126 from nucleotide 1 to nucleotide 338, to a nucleotide sequence corresponding to the 3′ end of said sequence of SEQ ID NO:126 from nucleotide 1 to nucleotide 338.

In other embodiments, the present invention provides a composition comprising a protein, wherein said protein comprises an amino acid sequence selected from the group consisting of:
(a) the amino acid sequence of SEQ ID NO:127;
(b) the amino acid sequence of SEQ ID NO:127 from amino acid 1 to amino acid 86;
(c) a fragment of the amino acid sequence of SEQ ID NO:127, the fragment comprising eight contiguous amino acids of SEQ ID NO:127; and
(d) the amino acid sequence encoded by the cDNA insert of clone BM46_{—}10 deposited with the ATCC under accession number 98152;
   the protein being substantially free from other mammalian proteins. Preferably such protein comprises the amino acid sequence of SEQ ID NO:127; the amino acid sequence of SEQ ID NO:127 from amino acid 1 to amino acid 86; or the amino acid sequence of SEQ ID NO:127 from amino acid 113 to amino acid 557. In further preferred embodiments, the present invention provides a protein comprising a fragment of the amino acid sequence of SEQ ID NO:127 having biological activity, the fragment preferably comprising eight (more preferably twenty, most preferably thirty) contiguous amino acids of SEQ ID NO:127, or a protein comprising a fragment of the amino acid sequence of SEQ ID NO:127 having biological activity, the fragment comprising the amino acid sequence from amino acid 282 to amino acid 291 of SEQ ID NO:127.

In one embodiment, the present invention provides a composition comprising an isolated polynucleotide selected from the group consisting of:
(a) a polynucleotide comprising the nucleotide sequence of SEQ ID NO:128;
(b) a polynucleotide comprising the nucleotide sequence of SEQ ID NO:128 from nucleotide 719 to nucleotide 886;
(c) a polynucleotide comprising the nucleotide sequence of SEQ ID NO:128 from nucleotide 812 to nucleotide 886;
(d) a polynucleotide comprising the nucleotide sequence of SEQ ID NO:128 from nucleotide 490 to nucleotide 853;
(e) a polynucleotide comprising the nucleotide sequence of the full-length protein coding sequence of clone J317_{—}1 deposited with the ATCC under accession number 98101;
(f) a polynucleotide encoding the full-length protein encoded by the cDNA insert of clone J317_{—}1 deposited with the ATCC under accession number 98101;
(g) a polynucleotide comprising the nucleotide sequence of a mature protein coding sequence of clone J317_{—}1 deposited with the ATCC under accession number 98101;
(h) a polynucleotide encoding a mature protein encoded by the cDNA insert of clone J317_{—}1 deposited with the ATCC under accession number 98101;
(i) a polynucleotide encoding a protein comprising the amino acid sequence of SEQ ID NO:129;
(j) a polynucleotide encoding a protein comprising a fragment of the amino acid sequence of SEQ ID NO:129 having biological activity, the fragment comprising eight contiguous amino acids of SEQ ID NO:129;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:128.

Preferably, such polynucleotide comprises the nucleotide sequence of SEQ ID NO:128 from nucleotide 719 to nucleotide 886; the nucleotide sequence of SEQ ID NO:128 from nucleotide 812 to nucleotide 886; the nucleotide sequence of SEQ ID NO:128 from nucleotide 490 to nucleotide 853; the nucleotide sequence of the full-length protein coding sequence of clone J317_{—}1 deposited with the ATCC under accession number 98101; or the nucleotide sequence of a mature protein coding sequence of clone J317_{—}1 deposited with the ATCC under accession number 98101. In other preferred embodiments, the polynucleotide encodes the full-length or a mature protein encoded by the cDNA insert of clone J317_{—}1 deposited with the ATCC under accession number 98101. In further preferred embodiments, the present invention provides a polynucleotide encoding a protein comprising a fragment of the amino acid sequence of SEQ ID NO:129 having biological activity, the fragment preferably comprising eight (more preferably twenty, most preferably thirty) contiguous amino acids of SEQ ID NO:129, or a polynucleotide encoding a protein comprising a fragment of the amino acid sequence of SEQ ID NO:129 having biological activity, the fragment comprising the amino acid sequence from amino acid 23 to amino acid 32 of SEQ ID NO:129.

Other embodiments provide the gene corresponding to the cDNA sequence of SEQ ID NO:128.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:128, but excluding the poly(A) tail at the 3′ end of SEQ ID NO:128; and
      (ab) the nucleotide sequence of the cDNA insert of clone J317_{—}1 deposited with the ATCC under accession number 98101;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:128, but excluding the poly(A) tail at the 3′ end of SEQ ID NO:128; and
      (bb) the nucleotide sequence of the cDNA insert of clone J317_{—}1 deposited with the ATCC under accession number 98101;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:128, and extending contiguously from a nucleotide sequence corresponding to the 5′ end of SEQ ID NO:128 to a nucleotide sequence corresponding to the 3′ end of SEQ ID NO:128, but excluding the poly(A) tail at the 3′ end of SEQ ID NO:128. Also preferably the polynucleotide isolated according to the above process comprises a nucleotide sequence corresponding to the cDNA sequence of SEQ ID NO:128 from nucleotide 719 to nucleotide 886, and extending contiguously from a nucleotide sequence corresponding to the 5′ end of said sequence of SEQ ID NO:128 from nucleotide 719 to nucleotide 886, to a nucleotide sequence corresponding to the 3′ end of said sequence of SEQ ID NO:128 from nucleotide 719 to nucleotide 886. Also preferably the polynucleotide isolated according to the above process comprises a nucleotide sequence corresponding to the cDNA sequence of SEQ ID NO:128 from nucleotide 812 to nucleotide 886, and extending contiguously from a nucleotide sequence corresponding to the 5′ end of said sequence of SEQ ID NO:128 from nucleotide 812 to nucleotide 886, to a nucleotide sequence corresponding to the 3′ end of said sequence of SEQ ID NO:128 from nucleotide 812 to nucleotide 886. Also preferably the polynucleotide isolated according to the above process comprises a nucleotide sequence corresponding to the cDNA sequence of SEQ ID NO:128 from nucleotide 490 to nucleotide 853, and extending contiguously from a nucleotide sequence corresponding to the 5′ end of said sequence of SEQ ID NO:128 from nucleotide 490 to nucleotide 853, to a nucleotide sequence corresponding to the 3′ end of said sequence of SEQ ID NO:128 from nucleotide 490 to nucleotide 853.

In other embodiments, the present invention provides a composition comprising a protein, wherein said protein comprises an amino acid sequence selected from the group consisting of:
(a) the amino acid sequence of SEQ ID NO:129;
(b) a fragment of the amino acid sequence of SEQ ID NO:129, the fragment comprising eight contiguous amino acids of SEQ ID NO:129; and
(c) the amino acid sequence encoded by the cDNA insert of clone J317_{—}1 deposited with the ATCC under accession number 98101;
   the protein being substantially free from other mammalian proteins. Preferably such protein comprises the amino acid sequence of SEQ ID NO:129. In further preferred embodiments, the present invention provides a protein comprising a fragment of the amino acid sequence of SEQ ID NO:129 having biological activity, the fragment preferably comprising eight (more preferably twenty, most preferably thirty) contiguous amino acids of SEQ ID NO:129, or a protein comprising a fragment of the amino acid sequence of SEQ ID NO:129 having biological activity, the fragment comprising the amino acid sequence from amino acid 23 to amino acid 32 of SEQ ID NO:129.

In one embodiment, the present invention provides a composition comprising an isolated polynucleotide selected from the group consisting of:
(a) a polynucleotide comprising the nucleotide sequence of SEQ ID NO:130;
(b) a polynucleotide comprising the nucleotide sequence of SEQ ID NO:130 from nucleotide 442 to nucleotide 609;
(c) a polynucleotide comprising the nucleotide sequence of SEQ ID NO:130 from nucleotide 269 to nucleotide 472;
(d) a polynucleotide comprising the nucleotide sequence of the full-length protein coding sequence of clone O289_{—}1 deposited with the ATCC under accession number 98101;
(e) a polynucleotide encoding the full-length protein encoded by the cDNA insert of clone O289_{—}1 deposited with the ATCC under accession number 98101;
(f) a polynucleotide comprising the nucleotide sequence of a mature protein coding sequence of clone O289_{—}1 deposited with the ATCC under accession number 98101;
(g) a polynucleotide encoding a mature protein encoded by the cDNA insert of clone O289_{—}1 deposited with the ATCC under accession number 98101;
(h) a polynucleotide encoding a protein comprising the amino acid sequence of SEQ ID NO:131;
(i) a polynucleotide encoding a protein comprising a fragment of the amino acid sequence of SEQ ID NO:131 having biological activity, the fragment comprising eight contiguous amino acids of SEQ ID NO:131;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:130.

Preferably, such polynucleotide comprises the nucleotide sequence of SEQ ID NO:130 from nucleotide 442 to nucleotide 609; the nucleotide sequence of SEQ ID NO:130 from nucleotide 269 to nucleotide 472; the nucleotide sequence of the full-length protein coding sequence of clone O289_{—}1 deposited with the ATCC under accession number 98101; or the nucleotide sequence of a mature protein coding sequence of clone O289_{—}1 deposited with the ATCC under accession number 98101. In other preferred embodiments, the polynucleotide encodes the full-length or a mature protein encoded by the cDNA insert of clone O289_{—}1 deposited with the ATCC under accession number 98101. In further preferred embodiments, the present invention provides a polynucleotide encoding a protein comprising a fragment of the amino acid sequence of SEQ ID NO:131 having biological activity, the fragment preferably comprising eight (more preferably twenty, most preferably thirty) contiguous amino acids of SEQ ID NO:131, or a polynucleotide encoding a protein comprising a fragment of the amino acid sequence of SEQ ID NO:131 having biological activity, the fragment comprising the amino acid sequence from amino acid 23 to amino acid 32 of SEQ ID NO:131.

Other embodiments provide the gene corresponding to the cDNA sequence of SEQ ID NO:130.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:130, but excluding the poly(A) tail at the 3′ end of SEQ ID NO:130; and
      (ab) the nucleotide sequence of the cDNA insert of clone O289_{—}1 deposited with the ATCC under accession number 98101;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:130, but excluding the poly(A) tail at the 3′ end of SEQ ID NO:130; and
      (bb) the nucleotide sequence of the cDNA insert of clone O289_{—}1 deposited with the ATCC under accession number 98101;
   (ii) hybridizing said primer(s) to human genomic DNA in conditions at least as stringent as 4×SSC at 50 degrees C.;
   (ii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:130, and extending contiguously from a nucleotide sequence corresponding to the 5′ end of SEQ ID NO:130 to a nucleotide sequence corresponding to the 3′ end of SEQ ID NO:130, but excluding the poly(A) tail at the 3′ end of SEQ ID NO:130. Also preferably the polynucleotide isolated according to the above process comprises a nucleotide sequence corresponding to the cDNA sequence of SEQ ID NO:130 from nucleotide 442 to nucleotide 609, and extending contiguously from a nucleotide sequence corresponding to the 5′ end of said sequence of SEQ ID NO:130 from nucleotide 442 to nucleotide 609, to a nucleotide sequence corresponding to the 3′ end of said sequence of SEQ ID NO:130 from nucleotide 442 to nucleotide 609. Also preferably the polynucleotide isolated according to the above process comprises a nucleotide sequence corresponding to the cDNA sequence of SEQ ID NO:130 from nucleotide 269 to nucleotide 472, and extending contiguously from a nucleotide sequence corresponding to the 5′ end of said sequence of SEQ ID NO:130 from nucleotide 269 to nucleotide 472, to a nucleotide sequence corresponding to the 3′ end of said sequence of SEQ ID NO:130 from nucleotide 269 to nucleotide 472.

In other embodiments, the present invention provides a composition comprising a protein, wherein said protein comprises an amino acid sequence selected from the group consisting of:
(a) the amino acid sequence of SEQ ID NO:131;
(b) a fragment of the amino acid sequence of SEQ ID NO:131, the fragment comprising eight contiguous amino acids of SEQ ID NO:131; and
(c) the amino acid sequence encoded by the cDNA insert of clone O289_{—}1 deposited with the ATCC under accession number 98101;
   the protein being substantially free from other mammalian proteins. Preferably such protein comprises the amino acid sequence of SEQ ID NO:131. In further preferred embodiments, the present invention provides a protein comprising a fragment of the amino acid sequence of SEQ ID NO:131 having biological activity, the fragment preferably comprising eight (more preferably twenty, most preferably thirty) contiguous amino acids of SEQ ID NO:131, or a protein comprising a fragment of the amino acid sequence of SEQ ID NO:131 having biological activity, the fragment comprising the amino acid sequence from amino acid 23 to amino acid 32 of SEQ ID NO:131.

In one embodiment, the present invention provides a composition comprising an isolated polynucleotide selected from the group consisting of:
(a) a polynucleotide comprising the nucleotide sequence of SEQ ID NO:132;
(b) a polynucleotide comprising the nucleotide sequence of SEQ ID NO:132 from nucleotide 44 to nucleotide 1204;
(c) a polynucleotide comprising the nucleotide sequence of SEQ ID NO:132 from nucleotide 24 to nucleotide 403;
(d) a polynucleotide comprising the nucleotide sequence of the full-length protein coding sequence of clone AJ26_{—}3 deposited with the ATCC under accession number 98115;
(e) a polynucleotide encoding the full-length protein encoded by the cDNA insert of clone AJ26_{—}3 deposited with the ATCC under accession number 98115;
(f) a polynucleotide comprising the nucleotide sequence of a mature protein coding sequence of clone AJ26_{—}3 deposited with the ATCC under accession number 98115;
(g) a polynucleotide encoding a mature protein encoded by the cDNA insert of clone AJ26_{—}3 deposited with the ATCC under accession number 98115;
(h) a polynucleotide encoding a protein comprising the amino acid sequence of SEQ ID NO:133;
(i) a polynucleotide encoding a protein comprising a fragment of the amino acid sequence of SEQ ID NO:133 having biological activity, the fragment comprising eight contiguous amino acids of SEQ ID NO:133;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:132.

Preferably, such polynucleotide comprises the nucleotide sequence of SEQ ID NO:132 from nucleotide 44 to nucleotide 1204; the nucleotide sequence of SEQ ID NO:132 from nucleotide 24 to nucleotide 403; the nucleotide sequence of the full-length protein coding sequence of clone AJ26_{—}3 deposited with the ATCC under accession number 98115; or the nucleotide sequence of a mature protein coding sequence of clone AJ26_{—}3 deposited with the ATCC under accession number 98115. In other preferred embodiments, the polynucleotide encodes the full-length or a mature protein encoded by the cDNA insert of clone AJ26_{—}3 deposited with the ATCC under accession number 98115. In yet other preferred embodiments, the present invention provides a polynucleotide encoding a protein comprising the amino acid sequence of SEQ ID NO:133 from amino acid 1 to amino acid 120. In further preferred embodiments, the present invention provides a polynucleotide encoding a protein comprising a fragment of the amino acid sequence of SEQ ID NO:133 having biological activity, the fragment preferably comprising eight (more preferably twenty, most preferably thirty) contiguous amino acids of SEQ ID NO:133, or a polynucleotide encoding a protein comprising a fragment of the amino acid sequence of SEQ ID NO:133 having biological activity, the fragment comprising the amino acid sequence from amino acid 188 to amino acid 197 of SEQ ID NO:133.

Other embodiments provide the gene corresponding to the cDNA sequence of SEQ ID NO:132.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:132, but excluding the poly(A) tail at the 3′ end of SEQ ID NO:132; and
      (ab) the nucleotide sequence of the cDNA insert of clone AJ26_{—}3 deposited with the ATCC under accession number 98115;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:132, but excluding the poly(A) tail at the 3′ end of SEQ ID NO:132; and
      (bb) the nucleotide sequence of the cDNA insert of clone AJ26_{—}3 deposited with the ATCC under accession number 98115;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:132, and extending contiguously from a nucleotide sequence corresponding to the 5′ end of SEQ ID NO:132 to a nucleotide sequence corresponding to the 3′ end of SEQ ID NO:132, but excluding the poly(A) tail at the 3′ end of SEQ ID NO:132. Also preferably the polynucleotide isolated according to the above process comprises a nucleotide sequence corresponding to the cDNA sequence of SEQ ID NO:132 from nucleotide 44 to nucleotide 1204, and extending contiguously from a nucleotide sequence corresponding to the 5′ end of said sequence of SEQ ID NO:132 from nucleotide 44 to nucleotide 1204, to a nucleotide sequence corresponding to the 3′ end of said sequence of SEQ ID NO:132 from nucleotide 44 to nucleotide 1204. Also preferably the polynucleotide isolated according to the above process comprises a nucleotide sequence corresponding to the cDNA sequence of SEQ ID NO:132 from nucleotide 24 to nucleotide 403, and extending contiguously from a nucleotide sequence corresponding to the 5′ end of said sequence of SEQ ID NO:132 from nucleotide 24 to nucleotide 403, to a nucleotide sequence corresponding to the 3′ end of said sequence of SEQ ID NO:132 from nucleotide 24 to nucleotide 403.

In other embodiments, the present invention provides a composition comprising a protein, wherein said protein comprises an amino acid sequence selected from the group consisting of:
(a) the amino acid sequence of SEQ ID NO:133;
(b) the amino acid sequence of SEQ ID NO:133 from amino acid 1 to amino acid 120;
(c) a fragment of the amino acid sequence of SEQ ID NO:133, the fragment comprising eight contiguous amino acids of SEQ ID NO:133; and
(d) the amino acid sequence encoded by the cDNA insert of clone AJ26_{—}3 deposited with the ATCC under accession number 98115;
   the protein being substantially free from other mammalian proteins. Preferably such protein comprises the amino acid sequence of SEQ ID NO:133 or the amino acid sequence of SEQ ID NO:133 from amino acid 1 to amino acid 120. In further preferred embodiments, the present invention provides a protein comprising a fragment of the amino acid sequence of SEQ ID NO:133 having biological activity, the fragment preferably comprising eight (more preferably twenty, most preferably thirty) contiguous amino acids of SEQ ID NO:133, or a protein comprising a fragment of the amino acid sequence of SEQ ID NO:133 having biological activity, the fragment comprising the amino acid sequence from amino acid 188 to amino acid 197 of SEQ ID NO:133.

In one embodiment, the present invention provides a composition comprising an isolated polynucleotide selected from the group consisting of:
(a) a polynucleotide comprising the nucleotide sequence of SEQ ID NO:134;
(b) a polynucleotide comprising the nucleotide sequence of SEQ ID NO:134 from nucleotide 928 to nucleotide 2541;
(c) a polynucleotide comprising the nucleotide sequence of SEQ ID NO:134 from nucleotide 988 to nucleotide 2541;
(d) a polynucleotide comprising the nucleotide sequence of SEQ ID NO:134 from nucleotide 738 to nucleotide 1128;
(e) a polynucleotide comprising the nucleotide sequence of the full-length protein coding sequence of clone AJ172_{—}2 deposited with the ATCC under accession number 98115;
(f) a polynucleotide encoding the full-length protein encoded by the cDNA insert of clone AJ172_{—}2 deposited with the ATCC under accession number 98115;
(g) a polynucleotide comprising the nucleotide sequence of a mature protein coding sequence of clone AJ172_{—}2 deposited with the ATCC under accession number 98115;
(h) a polynucleotide encoding a mature protein encoded by the cDNA insert of clone AJ172_{—}2 deposited with the ATCC under accession number 98115;
(i) a polynucleotide encoding a protein comprising the amino acid sequence of SEQ ID NO:135;
(j) a polynucleotide encoding a protein comprising a fragment of the amino acid sequence of SEQ ID NO:135 having biological activity, the fragment comprising eight contiguous amino acids of SEQ ID NO:135;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:134.

Preferably, such polynucleotide comprises the nucleotide sequence of SEQ ID NO:134 from nucleotide 928 to nucleotide 2541; the nucleotide sequence of SEQ ID NO:134 from nucleotide 988 to nucleotide 2541; the nucleotide sequence of SEQ ID NO:134 from nucleotide 738 to nucleotide 1128; the nucleotide sequence of the full-length protein coding sequence of clone AJ172_{—}2 deposited with the ATCC under accession number 98115; or the nucleotide sequence of a mature protein coding sequence of clone AJ172_{—}2 deposited with the ATCC under accession number 98115. In other preferred embodiments, the polynucleotide encodes the full-length or a mature protein encoded by the cDNA insert of clone AJ172_{—}2 deposited with the ATCC under accession number 98115. In yet other preferred embodiments, the present invention provides a polynucleotide encoding a protein comprising the amino acid sequence of SEQ ID NO:135 from amino acid 1 to amino acid 67. In further preferred embodiments, the present invention provides a polynucleotide encoding a protein comprising a fragment of the amino acid sequence of SEQ ID NO:135 having biological activity, the fragment preferably comprising eight (more preferably twenty, most preferably thirty) contiguous amino acids of SEQ ID NO:135, or a polynucleotide encoding a protein comprising a fragment of the amino acid sequence of SEQ ID NO:135 having biological activity, the fragment comprising the amino acid sequence from amino acid 264 to amino acid 273 of SEQ ID NO:135.

Other embodiments provide the gene corresponding to the cDNA sequence of SEQ ID NO:134.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:134, but excluding the poly(A) tail at the 3′ end of SEQ ID NO:134; and
      (ab) the nucleotide sequence of the cDNA insert of clone AJ172_{—}2 deposited with the ATCC under accession number 98115;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:134, but excluding the poly(A) tail at the 3′ end of SEQ ID NO:134; and
      (bb) the nucleotide sequence of the cDNA insert of clone AJ172_{—}2 deposited with the ATCC under accession number 98115;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:134, and extending contiguously from a nucleotide sequence corresponding to the 5′ end of SEQ ID NO:134 to a nucleotide sequence corresponding to the 3′ end of SEQ ID NO:134, but excluding the poly(A) tail at the 3′ end of SEQ ID NO:134. Also preferably the polynucleotide isolated according to the above process comprises a nucleotide sequence corresponding to the cDNA sequence of SEQ ID NO:134 from nucleotide 928 to nucleotide 2541, and extending contiguously from a nucleotide sequence corresponding to the 5′ end of said sequence of SEQ ID NO:134 from nucleotide 928 to nucleotide 2541, to a nucleotide sequence corresponding to the 3′ end of said sequence of SEQ ID NO:134 from nucleotide 928 to nucleotide 2541. Also preferably the polynucleotide isolated according to the above process comprises a nucleotide sequence corresponding to the cDNA sequence of SEQ ID NO:134 from nucleotide 988 to nucleotide 2541, and extending contiguously from a nucleotide sequence corresponding to the 5′ end of said sequence of SEQ ID NO:134 from nucleotide 988 to nucleotide 2541, to a nucleotide sequence corresponding to the 3′ end of said sequence of SEQ ID NO:134 from nucleotide 988 to nucleotide 2541. Also preferably the polynucleotide isolated according to the above process comprises a nucleotide sequence corresponding to the cDNA sequence of SEQ ID NO:134 from nucleotide 738 to nucleotide 1128, and extending contiguously from a nucleotide sequence corresponding to the 5′ end of said sequence of SEQ ID NO:134 from nucleotide 738 to nucleotide 1128, to a nucleotide sequence corresponding to the 3′ end of said sequence of SEQ ID NO:134 from nucleotide 738 to nucleotide 1128.

In other embodiments, the present invention provides a composition comprising a protein, wherein said protein comprises an amino acid sequence selected from the group consisting of:
(a) the amino acid sequence of SEQ ID NO:135;
(b) the amino acid sequence of SEQ ID NO:135 from amino acid 1 to amino acid 67;
(c) a fragment of the amino acid sequence of SEQ ID NO:135, the fragment comprising eight contiguous amino acids of SEQ ID NO:135; and
(d) the amino acid sequence encoded by the cDNA insert of clone AJ172_{—}2 deposited with the ATCC under accession number 98115;
   the protein being substantially free from other mammalian proteins. Preferably such protein comprises the amino acid sequence of SEQ ID NO:135 or the amino acid sequence of SEQ ID NO:135 from amino acid 1 to amino acid 67. In further preferred embodiments, the present invention provides a protein comprising a fragment of the amino acid sequence of SEQ ID NO:135 having biological activity, the fragment preferably comprising eight (more preferably twenty, most preferably thirty) contiguous amino acids of SEQ ID NO:135, or a protein comprising a fragment of the amino acid sequence of SEQ ID NO:135 having biological activity, the fragment comprising the amino acid sequence from amino acid 264 to amino acid 273 of SEQ ID NO:135.

In one embodiment, the present invention provides a composition comprising an isolated polynucleotide selected from the group consisting of:
(a) a polynucleotide comprising the nucleotide sequence of SEQ ID NO:136;
(b) a polynucleotide comprising the nucleotide sequence of SEQ ID NO:136 from nucleotide 523 to nucleotide 864;
(c) a polynucleotide comprising the nucleotide sequence of SEQ ID NO:136 from nucleotide 574 to nucleotide 864;
(d) a polynucleotide comprising the nucleotide sequence of the full-length protein coding sequence of clone AP224_{—}2 deposited with the ATCC under accession number 98115;
(e) a polynucleotide encoding the full-length protein encoded by the cDNA insert of clone AP224_{—}2 deposited with the ATCC under accession number 98115;
(f) a polynucleotide comprising the nucleotide sequence of a mature protein coding sequence of clone AP224_{—}2 deposited with the ATCC under accession number 98115;
(g) a polynucleotide encoding a mature protein encoded by the cDNA insert of clone AP224_{—}2 deposited with the ATCC under accession number 98115;
(h) a polynucleotide encoding a protein comprising the amino acid sequence of SEQ ID NO:137;
(i) a polynucleotide encoding a protein comprising a fragment of the amino acid sequence of SEQ ID NO:137 having biological activity, the fragment comprising eight contiguous amino acids of SEQ ID NO:137;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:136.

Preferably, such polynucleotide comprises the nucleotide sequence of SEQ ID NO:136 from nucleotide 523 to nucleotide 864; the nucleotide sequence of SEQ ID NO:136 from nucleotide 574 to nucleotide 864; the nucleotide sequence of the full-length protein coding sequence of clone AP224_{—}2 deposited with the ATCC under accession number 98115; or the nucleotide sequence of a mature protein coding sequence of clone AP224_{—}2 deposited with the ATCC under accession number 98115. In other preferred embodiments, the polynucleotide encodes the full-length or a mature protein encoded by the cDNA insert of clone AP224_{—}2 deposited with the ATCC under accession number 98115. In further preferred embodiments, the present invention provides a polynucleotide encoding a protein comprising a fragment of the amino acid sequence of SEQ ID NO:137 having biological activity, the fragment preferably comprising eight (more preferably twenty, most preferably thirty) contiguous amino acids of SEQ ID NO:137, or a polynucleotide encoding a protein comprising a fragment of the amino acid sequence of SEQ ID NO:137 having biological activity, the fragment comprising the amino acid sequence from amino acid 52 to amino acid 61 of SEQ ID NO:137.

Other embodiments provide the gene corresponding to the cDNA sequence of SEQ ID NO:136.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:136, but excluding the poly(A) tail at the 3′ end of SEQ ID NO:136; and
      (ab) the nucleotide sequence of the cDNA insert of clone AP224_{—}2 deposited with the ATCC under accession number 98115;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:136, but excluding the poly(A) tail at the 3′ end of SEQ ID NO:136; and
      (bb) the nucleotide sequence of the cDNA insert of clone AP224_{—}2 deposited with the ATCC under accession number 98115;
   (ii) hybridizing said primer(s) to human genomic DNA in conditions at least as stringent as 4×SSC at 50 degrees C.;
   (ii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:136, and extending contiguously from a nucleotide sequence corresponding to the 5′ end of SEQ ID NO:136 to a nucleotide sequence corresponding to the 3′ end of SEQ ID NO:136, but excluding the poly(A) tail at the 3′ end of SEQ ID NO:136. Also preferably the polynucleotide isolated according to the above process comprises a nucleotide sequence corresponding to the cDNA sequence of SEQ ID NO:136 from nucleotide 523 to nucleotide 864, and extending contiguously from a nucleotide sequence corresponding to the 5′ end of said sequence of SEQ ID NO:136 from nucleotide 523 to nucleotide 864, to a nucleotide sequence corresponding to the 3′ end of said sequence of SEQ ID NO:136 from nucleotide 523 to nucleotide 864. Also preferably the polynucleotide isolated according to the above process comprises a nucleotide sequence corresponding to the cDNA sequence of SEQ ID NO:136 from nucleotide 574 to nucleotide 864, and extending contiguously from a nucleotide sequence corresponding to the 5′ end of said sequence of SEQ ID NO:136 from nucleotide 574 to nucleotide 864, to a nucleotide sequence corresponding to the 3′ end of said sequence of SEQ ID NO:136 from nucleotide 574 to nucleotide 864.

In other embodiments, the present invention provides a composition comprising a protein, wherein said protein comprises an amino acid sequence selected from the group consisting of:
(a) the amino acid sequence of SEQ ID NO:137;
(b) a fragment of the amino acid sequence of SEQ ID NO:137, the fragment comprising eight contiguous amino acids of SEQ ID NO:137; and
(c) the amino acid sequence encoded by the cDNA insert of clone AP224_{—}2 deposited with the ATCC under accession number 98115;
   the protein being substantially free from other mammalian proteins. Preferably such protein comprises the amino acid sequence of SEQ ID NO:137. In further preferred embodiments, the present invention provides a protein comprising a fragment of the amino acid sequence of SEQ ID NO:137 having biological activity, the fragment preferably comprising eight (more preferably twenty, most preferably thirty) contiguous amino acids of SEQ ID NO:137, or a protein comprising a fragment of the amino acid sequence of SEQ ID NO:137 having biological activity, the fragment comprising the amino acid sequence from amino acid 52 to amino acid 61 of SEQ ID NO:137.

In one embodiment, the present invention provides a composition comprising an isolated polynucleotide selected from the group consisting of:
(a) a polynucleotide comprising the nucleotide sequence of SEQ ID NO:138;
(b) a polynucleotide comprising the nucleotide sequence of SEQ ID NO:138 from nucleotide 6 to nucleotide 2408;
(c) a polynucleotide comprising the nucleotide sequence of SEQ ID NO:138 from nucleotide 1437 to nucleotide 1705;
(d) a polynucleotide comprising the nucleotide sequence of the full-length protein coding sequence of clone BL89_{—}13 deposited with the ATCC under accession number 98153;
(e) a polynucleotide encoding the full-length protein encoded by the cDNA insert of clone BL89_{—}13 deposited with the ATCC under accession number 98153;
(f) a polynucleotide comprising the nucleotide sequence of a mature protein coding sequence of clone BL89_{—}13 deposited with the ATCC under accession number 98153;
(g) a polynucleotide encoding a mature protein encoded by the cDNA insert of clone BL89_{—}13 deposited with the ATCC under accession number 98153;
(h) a polynucleotide encoding a protein comprising the amino acid sequence of SEQ ID NO:139;
(i) a polynucleotide encoding a protein comprising a fragment of the amino acid sequence of SEQ ID NO:139 having biological activity, the fragment comprising eight contiguous amino acids of SEQ ID NO:139;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:138.

Preferably, such polynucleotide comprises the nucleotide sequence of SEQ ID NO:138 from nucleotide 6 to nucleotide 2408; the nucleotide sequence of SEQ ID NO:138 from nucleotide 1437 to nucleotide 1705; the nucleotide sequence of the full-length protein coding sequence of clone BL89_{—}13 deposited with the ATCC under accession number 98153; or the nucleotide sequence of a mature protein coding sequence of clone BL89_{—}13 deposited with the ATCC under accession number 98153. In other preferred embodiments, the polynucleotide encodes the full-length or a mature protein encoded by the cDNA insert of clone BL89_{—}13 deposited with the ATCC under accession number 98153. In yet other preferred embodiments, the present invention provides a polynucleotide encoding a protein comprising the amino acid sequence of SEQ ID NO:139 from amino acid 431 to amino acid 567. In further preferred embodiments, the present invention provides a polynucleotide encoding a protein comprising a fragment of the amino acid sequence of SEQ ID NO:139 having biological activity, the fragment preferably comprising eight (more preferably twenty, most preferably thirty) contiguous amino acids of SEQ ID NO:139, or a polynucleotide encoding a protein comprising a fragment of the amino acid sequence of SEQ ID NO:139 having biological activity, the fragment comprising the amino acid sequence from amino acid 395 to amino acid 404 of SEQ ID NO:139.

Other embodiments provide the gene corresponding to the cDNA sequence of SEQ ID NO:138.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:138, but excluding the poly(A) tail at the 3′ end of SEQ ID NO:138; and
      (ab) the nucleotide sequence of the cDNA insert of clone BL89_{—}13 deposited with the ATCC under accession number 98153;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:138, but excluding the poly(A) tail at the 3′ end of SEQ ID NO:138; and
      (bb) the nucleotide sequence of the cDNA insert of clone BL89_{—}13 deposited with the ATCC under accession number 98153;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:138, and extending contiguously from a nucleotide sequence corresponding to the 5′ end of SEQ ID NO:138 to a nucleotide sequence corresponding to the 3′ end of SEQ ID NO:138, but excluding the poly(A) tail at the 3′ end of SEQ ID NO:138. Also preferably the polynucleotide isolated according to the above process comprises a nucleotide sequence corresponding to the cDNA sequence of SEQ ID NO:138 from nucleotide 6 to nucleotide 2408, and extending contiguously from a nucleotide sequence corresponding to the 5′ end of said sequence of SEQ ID NO:138 from nucleotide 6 to nucleotide 2408, to a nucleotide sequence corresponding to the 3′ end of said sequence of SEQ ID NO:138 from nucleotide 6 to nucleotide 2408. Also preferably the polynucleotide isolated according to the above process comprises a nucleotide sequence corresponding to the cDNA sequence of SEQ ID NO:138 from nucleotide 1437 to nucleotide 1705, and extending contiguously from a nucleotide sequence corresponding to the 5′ end of said sequence of SEQ ID NO:138 from nucleotide 1437 to nucleotide 1705, to a nucleotide sequence corresponding to the 3′ end of said sequence of SEQ ID NO:138 from nucleotide 1437 to nucleotide 1705.

In other embodiments, the present invention provides a composition comprising a protein, wherein said protein comprises an amino acid sequence selected from the group consisting of:
(a) the amino acid sequence of SEQ ID NO:139;
(b) the amino acid sequence of SEQ ID NO:139 from amino acid 431 to amino acid 567;
(c) a fragment of the amino acid sequence of SEQ ID NO:139, the fragment comprising eight contiguous amino acids of SEQ ID NO:139; and
(d) the amino acid sequence encoded by the cDNA insert of clone BL89_{—}13 deposited with the ATCC under accession number 98153;
   the protein being substantially free from other mammalian proteins. Preferably such protein comprises the amino acid sequence of SEQ ID NO:139 or the amino acid sequence of SEQ ID NO:139 from amino acid 431 to amino acid 567. In further preferred embodiments, the present invention provides a protein comprising a fragment of the amino acid sequence of SEQ ID NO:139 having biological activity, the fragment preferably comprising eight (more preferably twenty, most preferably thirty) contiguous amino acids of SEQ ID NO:139, or a protein comprising a fragment of the amino acid sequence of SEQ ID NO:139 having biological activity, the fragment comprising the amino acid sequence from amino acid 395 to amino acid 404 of SEQ ID NO:139.

In one embodiment, the present invention provides a composition comprising an isolated polynucleotide selected from the group consisting of:
(a) a polynucleotide comprising the nucleotide sequence of SEQ ID NO:140;
(b) a polynucleotide comprising the nucleotide sequence of SEQ ID NO:140 from nucleotide 2113 to nucleotide 2337;
(c) a polynucleotide comprising the nucleotide sequence of SEQ ID NO:140 from nucleotide 2036 to nucleotide 2316;
(d) a polynucleotide comprising the nucleotide sequence of the full-length protein coding sequence of clone BL341_{—}4 deposited with the ATCC under accession number 98115;
(e) a polynucleotide encoding the full-length protein encoded by the cDNA insert of clone BL341_{—}4 deposited with the ATCC under accession number 98115;
(f) a polynucleotide comprising the nucleotide sequence of a mature protein coding sequence of clone BL341_{—}4 deposited with the ATCC under accession number 98115;
(g) a polynucleotide encoding a mature protein encoded by the cDNA insert of clone BL341_{—}4 deposited with the ATCC under accession number 98115;
(h) a polynucleotide encoding a protein comprising the amino acid sequence of SEQ ID NO:141;
(i) a polynucleotide encoding a protein comprising a fragment of the amino acid sequence of SEQ ID NO:141 having biological activity, the fragment comprising eight contiguous amino acids of SEQ ID NO:141;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:140.

Preferably, such polynucleotide comprises the nucleotide sequence of SEQ ID NO:140 from nucleotide 2113 to nucleotide 2337; the nucleotide sequence of SEQ ID NO:140 from nucleotide 2036 to nucleotide 2316; the nucleotide sequence of the full-length protein coding sequence of clone BL341_{—}4 deposited with the ATCC under accession number 98115; or the nucleotide sequence of a mature protein coding sequence of clone BL341_{—}4 deposited with the ATCC under accession number 98115. In other preferred embodiments, the polynucleotide encodes the full-length or a mature protein encoded by the cDNA insert of clone BL341_{—}4 deposited with the ATCC under accession number 98115. In yet other preferred embodiments, the present invention provides a polynucleotide encoding a protein comprising the amino acid sequence of SEQ ID NO:141 from amino acid 1 to amino acid 68. In further preferred embodiments, the present invention provides a polynucleotide encoding a protein comprising a fragment of the amino acid sequence of SEQ ID NO:141 having biological activity, the fragment preferably comprising eight (more preferably twenty, most preferably thirty) contiguous amino acids of SEQ ID NO:141, or a polynucleotide encoding a protein comprising a fragment of the amino acid sequence of SEQ ID NO:141 having biological activity, the fragment comprising the amino acid sequence from amino acid 32 to amino acid 41 of SEQ ID NO:141.

Other embodiments provide the gene corresponding to the cDNA sequence of SEQ ID NO:140.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:140, but excluding the poly(A) tail at the 3′ end of SEQ ID NO:140; and
      (ab) the nucleotide sequence of the cDNA insert of clone BL341_{—}4 deposited with the ATCC under accession number 98115;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:140, but excluding the poly(A) tail at the 3′ end of SEQ ID NO:140; and
      (bb) the nucleotide sequence of the cDNA insert of clone BL341_{—}4 deposited with the ATCC under accession number 98115;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:140, and extending contiguously from a nucleotide sequence corresponding to the 5′ end of SEQ ID NO:140 to a nucleotide sequence corresponding to the 3′ end of SEQ ID NO:140, but excluding the poly(A) tail at the 3′ end of SEQ ID NO:140. Also preferably the polynucleotide isolated according to the above process comprises a nucleotide sequence corresponding to the cDNA sequence of SEQ ID NO:140 from nucleotide 2113 to nucleotide 2337, and extending contiguously from a nucleotide sequence corresponding to the 5′ end of said sequence of SEQ ID NO:140 from nucleotide 2113 to nucleotide 2337, to a nucleotide sequence corresponding to the 3′ end of said sequence of SEQ ID NO:140 from nucleotide 2113 to nucleotide 2337. Also preferably the polynucleotide isolated according to the above process comprises a nucleotide sequence corresponding to the cDNA sequence of SEQ ID NO:140 from nucleotide 2036 to nucleotide 2316, and extending contiguously from a nucleotide sequence corresponding to the 5′ end of said sequence of SEQ ID NO:140 from nucleotide 2036 to nucleotide 2316, to a nucleotide sequence corresponding to the 3′ end of said sequence of SEQ ID NO:140 from nucleotide 2036 to nucleotide 2316.

In other embodiments, the present invention provides a composition comprising a protein, wherein said protein comprises an amino acid sequence selected from the group consisting of:
(a) the amino acid sequence of SEQ ID NO:141;
(b) the amino acid sequence of SEQ ID NO:141 from amino acid 1 to amino acid 68;
(c) a fragment of the amino acid sequence of SEQ ID NO:141, the fragment comprising eight contiguous amino acids of SEQ ID NO:141; and
(d) the amino acid sequence encoded by the cDNA insert of clone BL341_{—}4 deposited with the ATCC under accession number 98115;
   the protein being substantially free from other mammalian proteins. Preferably such protein comprises the amino acid sequence of SEQ ID NO:141 or the amino acid sequence of SEQ ID NO:141 from amino acid 1 to amino acid 68. In further preferred embodiments, the present invention provides a protein comprising a fragment of the amino acid sequence of SEQ ID NO:141 having biological activity, the fragment preferably comprising eight (more preferably twenty, most preferably thirty) contiguous amino acids of SEQ ID NO:141, or a protein comprising a fragment of the amino acid sequence of SEQ ID NO:141 having biological activity, the fragment comprising the amino acid sequence from amino acid 32 to amino acid 41 of SEQ ID NO:141.

In one embodiment, the present invention provides a composition comprising an isolated polynucleotide selected from the group consisting of:
(a) a polynucleotide comprising the nucleotide sequence of SEQ ID NO:142;
(b) a polynucleotide comprising the nucleotide sequence of SEQ ID NO:142 from nucleotide 92 to nucleotide 262;
(c) a polynucleotide comprising the nucleotide sequence of SEQ ID NO:142 from nucleotide 215 to nucleotide 262;
(d) a polynucleotide comprising the nucleotide sequence of SEQ ID NO:142 from nucleotide 15 to nucleotide 257;
(e) a polynucleotide comprising the nucleotide sequence of the full-length protein coding sequence of clone BV239_{—}3 deposited with the ATCC under accession number 98153;
(f) a polynucleotide encoding the full-length protein encoded by the cDNA insert of clone BV239_{—}3 deposited with the ATCC under accession number 98153;
(g) a polynucleotide comprising the nucleotide sequence of a mature protein coding sequence of clone BV239_{—}3 deposited with the ATCC under accession number 98153;
(h) a polynucleotide encoding a mature protein encoded by the cDNA insert of clone BV239_{—}3 deposited with the ATCC under accession number 98153;
(i) a polynucleotide encoding a protein comprising the amino acid sequence of SEQ ID NO:143;
(j) a polynucleotide encoding a protein comprising a fragment of the amino acid sequence of SEQ ID NO:143 having biological activity, the fragment comprising eight contiguous amino acids of SEQ ID NO:143;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:142.

Preferably, such polynucleotide comprises the nucleotide sequence of SEQ ID NO:142 from nucleotide 92 to nucleotide 262; the nucleotide sequence of SEQ ID NO:142 from nucleotide 215 to nucleotide 262; the nucleotide sequence of SEQ ID NO:142 from nucleotide 15 to nucleotide 257; the nucleotide sequence of the full-length protein coding sequence of clone BV239_{—}3 deposited with the ATCC under accession number 98153; or the nucleotide sequence of a mature protein coding sequence of clone BV239_{—}3 deposited with the ATCC under accession number 98153. In other preferred embodiments, the polynucleotide encodes the full-length or a mature protein encoded by the cDNA insert of clone BV239_{—}3 deposited with the ATCC under accession number 98153. In further preferred embodiments, the present invention provides a polynucleotide encoding a protein comprising a fragment of the amino acid sequence of SEQ ID NO:143 having biological activity, the fragment preferably comprising eight (more preferably twenty, most preferably thirty) contiguous amino acids of SEQ ID NO:143, or a polynucleotide encoding a protein comprising a fragment of the amino acid sequence of SEQ ID NO:143 having biological activity, the fragment comprising the amino acid sequence from amino acid 23 to amino acid 32 of SEQ ID NO:143.

Other embodiments provide the gene corresponding to the cDNA sequence of SEQ ID NO:142.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:142, but excluding the poly(A) tail at the 3′ end of SEQ ID NO:142; and
      (ab) the nucleotide sequence of the cDNA insert of clone BV239_{—}3 deposited with the ATCC under accession number 98153;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:142, but excluding the poly(A) tail at the 3′ end of SEQ ID NO:142; and
      (bb) the nucleotide sequence of the cDNA insert of clone BV239_{—}3 deposited with the ATCC under accession number 98153;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:142, and extending contiguously from a nucleotide sequence corresponding to the 5′ end of SEQ ID NO:142 to a nucleotide sequence corresponding to the 3′ end of SEQ ID NO:142, but excluding the poly(A) tail at the 3′ end of SEQ ID NO:142. Also preferably the polynucleotide isolated according to the above process comprises a nucleotide sequence corresponding to the cDNA sequence of SEQ ID NO:142 from nucleotide 92 to nucleotide 262, and extending contiguously from a nucleotide sequence corresponding to the 5′ end of said sequence of SEQ ID NO:142 from nucleotide 92 to nucleotide 262, to a nucleotide sequence corresponding to the 3′ end of said sequence of SEQ ID NO:142 from nucleotide 92 to nucleotide 262. Also preferably the polynucleotide isolated according to the above process comprises a nucleotide sequence corresponding to the cDNA sequence of SEQ ID NO:142 from nucleotide 215 to nucleotide 262, and extending contiguously from a nucleotide sequence corresponding to the 5′ end of said sequence of SEQ ID NO:142 from nucleotide 215 to nucleotide 262, to a nucleotide sequence corresponding to the 3′ end of said sequence of SEQ ID NO:142 from nucleotide 215 to nucleotide 262. Also preferably the polynucleotide isolated according to the above process comprises a nucleotide sequence corresponding to the cDNA sequence of SEQ ID NO:142 from nucleotide 15 to nucleotide 257, and extending contiguously from a nucleotide sequence corresponding to the 5′ end of said sequence of SEQ ID NO:142 from nucleotide 15 to nucleotide 257, to a nucleotide sequence corresponding to the 3′ end of said sequence of SEQ ID NO:142 from nucleotide 15 to nucleotide 257.

In other embodiments, the present invention provides a composition comprising a protein, wherein said protein comprises an amino acid sequence selected from the group consisting of:
(a) the amino acid sequence of SEQ ID NO:143;
(b) a fragment of the amino acid sequence of SEQ ID NO:143, the fragment comprising eight contiguous amino acids of SEQ ID NO:143; and
(c) the amino acid sequence encoded by the cDNA insert of clone BV239_{—}3 deposited with the ATCC under accession number 98153;
   the protein being substantially free from other mammalian proteins. Preferably such protein comprises the amino acid sequence of SEQ ID NO:143. In further preferred embodiments, the present invention provides a protein comprising a fragment of the amino acid sequence of SEQ ID NO:143 having biological activity, the fragment preferably comprising eight (more preferably twenty, most preferably thirty) contiguous amino acids of SEQ ID NO:143, or a protein comprising a fragment of the amino acid sequence of SEQ ID NO:143 having biological activity, the fragment comprising the amino acid sequence from amino acid 23 to amino acid 32 of SEQ ID NO:143.

In one embodiment, the present invention provides a composition comprising an isolated polynucleotide selected from the group consisting of:
(a) a polynucleotide comprising the nucleotide sequence of SEQ ID NO:144;
(b) a polynucleotide comprising the nucleotide sequence of SEQ ID NO:144 from nucleotide 144 to nucleotide 257;
(c) a polynucleotide comprising the nucleotide sequence of SEQ ID NO:144 from nucleotide 88 to nucleotide 271;
(d) a polynucleotide comprising the nucleotide sequence of the full-length protein coding sequence of clone CC25_{—}17 deposited with the ATCC under accession number 98153;
(e) a polynucleotide encoding the full-length protein encoded by the cDNA insert of clone CC25_{—}17 deposited with the ATCC under accession number 98153;
(f) a polynucleotide comprising the nucleotide sequence of a mature protein coding sequence of clone CC25_{—}17 deposited with the ATCC under accession number 98153;
(g) a polynucleotide encoding a mature protein encoded by the cDNA insert of clone CC25_{—}17 deposited with the ATCC under accession number 98153;
(h) a polynucleotide encoding a protein comprising the amino add sequence of SEQ ID NO:145;
(i) a polynucleotide encoding a protein comprising a fragment of the amino acid sequence of SEQ ID NO:145 having biological activity, the fragment comprising eight contiguous amino acids of SEQ ID NO:145;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:144.

Preferably, such polynucleotide comprises the nucleotide sequence of SEQ ID NO:144 from nucleotide 144 to nucleotide 257; the nucleotide sequence of SEQ ID NO:144 from nucleotide 88 to nucleotide 271; the nucleotide sequence of the full-length protein coding sequence of clone CC25_{—}17 deposited with the ATCC under accession number 98153; or the nucleotide sequence of a mature protein coding sequence of clone CC25_{—}17 deposited with the ATCC under accession number 98153. In other preferred embodiments, the polynucleotide encodes the full-length or a mature protein encoded by the cDNA insert of clone CC25_{—}17 deposited with the ATCC under accession number 98153. In further preferred embodiments, the present invention provides a polynucleotide encoding a protein comprising a fragment of the amino acid sequence of SEQ ID NO:145 having biological activity, the fragment preferably comprising eight (more preferably twenty, most preferably thirty) contiguous amino acids of SEQ ID NO:145, or a polynucleotide encoding a protein comprising a fragment of the amino acid sequence of SEQ ID NO:145 having biological activity, the fragment comprising the amino acid sequence from amino acid 14 to amino acid 23 of SEQ ID NO:145.

Other embodiments provide the gene corresponding to the cDNA sequence of SEQ ID NO:144.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:144, but excluding the poly(A) tail at the 3′ end of SEQ ID NO:144; and
      (ab) the nucleotide sequence of the cDNA insert of clone CC25_{—}17 deposited with the ATCC under accession number 98153;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:144, but excluding the poly(A) tail at the 3′ end of SEQ ID NO:144; and
      (bb) the nucleotide sequence of the cDNA insert of clone CC25_{—}17 deposited with the ATCC under accession number 98153;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:144, and extending contiguously from a nucleotide sequence corresponding to the 5′ end of SEQ ID NO:144 to a nucleotide sequence corresponding to the 3′ end of SEQ ID NO:144, but excluding the poly(A) tail at the 3′ end of SEQ ID NO:144. Also preferably the polynucleotide isolated according to the above process comprises a nucleotide sequence corresponding to the cDNA sequence of SEQ ID NO:144 from nucleotide 144 to nucleotide 257, and extending contiguously from a nucleotide sequence corresponding to the 5′ end of said sequence of SEQ ID NO:144 from nucleotide 144 to nucleotide 257, to a nucleotide sequence corresponding to the 3′ end of said sequence of SEQ ID NO:144 from nucleotide 144 to nucleotide 257. Also preferably the polynucleotide isolated according to the above process comprises a nucleotide sequence corresponding to the cDNA sequence of SEQ ID NO:144 from nucleotide 88 to nucleotide 271, and extending contiguously from a nucleotide sequence corresponding to the 5′ end of said sequence of SEQ ID NO:144 from nucleotide 88 to nucleotide 271, to a nucleotide sequence corresponding to the 3′ end of said sequence of SEQ ID NO:144 from nucleotide 88 to nucleotide 271.

In other embodiments, the present invention provides a composition comprising a protein, wherein said protein comprises an amino acid sequence selected from the group consisting of:
(a) the amino acid sequence of SEQ ID NO:145;
(b) a fragment of the amino acid sequence of SEQ ID NO:145, the fragment comprising eight contiguous amino acids of SEQ ID NO:145; and
(c) the amino acid sequence encoded by the cDNA insert of clone CC25_{—}17 deposited with the ATCC under accession number 98153;
   the protein being substantially free from other mammalian proteins. Preferably such protein comprises the amino acid sequence of SEQ ID NO:145. In further preferred embodiments, the present invention provides a protein comprising a fragment of the amino acid sequence of SEQ ID NO:145 having biological activity, the fragment preferably comprising eight (more preferably twenty, most preferably thirty) contiguous amino acids of SEQ ID NO:145, or a protein comprising a fragment of the amino acid sequence of SEQ ID NO:145 having biological activity, the fragment comprising the amino acid sequence from amino acid 14 to amino acid 23 of SEQ ID NO:145.

In one embodiment, the present invention provides a composition comprising an isolated polynucleotide selected from the group consisting of:
(a) a polynucleotide comprising the nucleotide sequence of SEQ ID NO:146;
(b) a polynucleotide comprising the nucleotide sequence of SEQ ID NO:146 from nucleotide 431 to nucleotide 520;
(c) a polynucleotide comprising the nucleotide sequence of SEQ ID NO:146 from nucleotide 380 to nucleotide 511;
(d) a polynucleotide comprising the nucleotide sequence of the full-length protein coding sequence of clone CC397_{—}19 deposited with the ATCC under accession number 98153;
(e) a polynucleotide encoding the full-length protein encoded by the cDNA insert of clone CC397_{—}19 deposited with the ATCC under accession number 98153;
(f) a polynucleotide comprising the nucleotide sequence of a mature protein coding sequence of clone CC397_{—}19 deposited with the ATCC under accession number 98153;
(g) a polynucleotide encoding a mature protein encoded by the cDNA insert of clone CC397_{—}19 deposited with the ATCC under accession number 98153;
(h) a polynucleotide encoding a protein comprising the amino acid sequence of SEQ ID NO:147;
(i) a polynucleotide encoding a protein comprising a fragment of the amino acid sequence of SEQ ID NO:147 having biological activity, the fragment comprising eight contiguous amino acids of SEQ ID NO:147;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:146.

Preferably, such polynucleotide comprises the nucleotide sequence of SEQ ID NO:146 from nucleotide 431 to nucleotide 520; the nucleotide sequence of SEQ ID NO:146 from nucleotide 380 to nucleotide 511; the nucleotide sequence of the full-length protein coding sequence of clone CC397_{—}19 deposited with the ATCC under accession number 98153; or the nucleotide sequence of a mature protein coding sequence of clone CC397_{—}19 deposited with the ATCC under accession number 98153. In other preferred embodiments, the polynucleotide encodes the full-length or a mature protein encoded by the cDNA insert of clone CC397_{—}19 deposited with the ATCC under accession number 98153. In yet other preferred embodiments, the present invention provides a polynucleotide encoding a protein comprising the amino acid sequence of SEQ ID NO:147 from amino acid 1 to amino acid 27. In further preferred embodiments, the present invention provides a polynucleotide encoding a protein comprising a fragment of the amino acid sequence of SEQ ID NO:147 having biological activity, the fragment preferably comprising eight (more preferably twenty, most preferably thirty) contiguous amino acids of SEQ ID NO:147, or a polynucleotide encoding a protein comprising a fragment of the amino acid sequence of SEQ ID NO:147 having biological activity, the fragment comprising the amino acid sequence from amino acid 10 to amino acid 19 of SEQ ID NO:147.

Other embodiments provide the gene corresponding to the cDNA sequence of SEQ ID NO:146.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:146, but excluding the poly(A) tail at the 3′ end of SEQ ID NO:146; and
      (ab) the nucleotide sequence of the cDNA insert of clone CC397_{—}19 deposited with the ATCC under accession number 98153;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:146, but excluding the poly(A) tail at the 3′ end of SEQ ID NO:146; and
      (bb) the nucleotide sequence of the cDNA insert of clone CC397_{—}19 deposited with the ATCC under accession number 98153;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:146, and extending contiguously from a nucleotide sequence corresponding to the 5′ end of SEQ ID NO:146 to a nucleotide sequence corresponding to the 3′ end of SEQ ID NO:146, but excluding the poly(A) tail at the 3′ end of SEQ ID NO:146. Also preferably the polynucleotide isolated according to the above process comprises a nucleotide sequence corresponding to the cDNA sequence of SEQ ID NO:146 from nucleotide 431 to nucleotide 520, and extending contiguously from a nucleotide sequence corresponding to the 5′ end of said sequence of SEQ ID NO:146 from nucleotide 431 to nucleotide 520, to a nucleotide sequence corresponding to the 3′ end of said sequence of SEQ ID NO:146 from nucleotide 431 to nucleotide 520. Also preferably the polynucleotide isolated according to the above process comprises a nucleotide sequence corresponding to the cDNA sequence of SEQ ID NO:146 from nucleotide 380 to nucleotide 511, and extending contiguously from a nucleotide sequence corresponding to the 5′ end of said sequence of SEQ ID NO:146 from nucleotide 380 to nucleotide 511, to a nucleotide sequence corresponding to the 3′ end of said sequence of SEQ ID NO:146 from nucleotide 380 to nucleotide 511.

In other embodiments, the present invention provides a composition comprising a protein, wherein said protein comprises an amino acid sequence selected from the group consisting of:
(a) the amino acid sequence of SEQ ID NO:147;
(b) the amino acid sequence of SEQ ID NO:147 from amino acid 1 to amino acid 27;
(c) a fragment of the amino acid sequence of SEQ ID NO:147, the fragment comprising eight contiguous amino acids of SEQ ID NO:147; and
(d) the amino acid sequence encoded by the cDNA insert of clone CC397_{—}19 deposited with the ATCC under accession number 98153;
   the protein being substantially free from other mammalian proteins. Preferably such protein comprises the amino acid sequence of SEQ ID NO:147 or the amino acid sequence of SEQ ID NO:147 from amino acid 1 to amino acid 27. In further preferred embodiments, the present invention provides a protein comprising a fragment of the amino acid sequence of SEQ ID NO:147 having biological activity, the fragment preferably comprising eight (more preferably twenty, most preferably thirty) contiguous amino acids of SEQ ID NO:147, or a protein comprising a fragment of the amino acid sequence of SEQ ID NO:147 having biological activity, the fragment comprising the amino acid sequence from amino acid 10 to amino acid 19 of SEQ ID NO:147.

In one embodiment, the present invention provides a composition comprising an isolated polynucleotide selected from the group consisting of:
(a) a polynucleotide comprising the nucleotide sequence of SEQ ID NO:149;
(b) a polynucleotide comprising the nucleotide sequence of SEQ ID NO:149 from nucleotide 253 to nucleotide 519;
(c) a polynucleotide comprising the nucleotide sequence of SEQ ID NO:149 from nucleotide 298 to nucleotide 519;
(d) a polynucleotide comprising the nucleotide sequence of the full-length protein coding sequence of clone D305_{—}2 deposited with the ATCC under accession number 98115;
(e) a polynucleotide encoding the full-length protein encoded by the cDNA insert of clone D305_{—}2 deposited with the ATCC under accession number 98115;
(f) a polynucleotide comprising the nucleotide sequence of a mature protein coding sequence of clone D305_{—}2 deposited with the ATCC under accession number 98115;
(g) a polynucleotide encoding a mature protein encoded by the cDNA insert of clone D305_{—}2 deposited with the ATCC under accession number 98115;
(h) a polynucleotide encoding a protein comprising the amino acid sequence of SEQ ID NO:150;
(i) a polynucleotide encoding a protein comprising a fragment of the amino acid sequence of SEQ ID NO:150 having biological activity, the fragment comprising eight contiguous amino acids of SEQ ID NO:150;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:149.

Preferably, such polynucleotide comprises the nucleotide sequence of SEQ ID NO:149 from nucleotide 253 to nucleotide 519; the nucleotide sequence of SEQ ID NO:149 from nucleotide 298 to nucleotide 519; the nucleotide sequence of the full-length protein coding sequence of clone D305_{—}2 deposited with the ATCC under accession number 98115; or the nucleotide sequence of a mature protein coding sequence of clone D305_{—}2 deposited with the ATCC under accession number 98115. In other preferred embodiments, the polynucleotide encodes the full-length or a mature protein encoded by the cDNA insert of clone D305_{—}2 deposited with the ATCC under accession number 98115. In further preferred embodiments, the present invention provides a polynucleotide encoding a protein comprising a fragment of the amino acid sequence of SEQ ID NO:150 having biological activity, the fragment preferably comprising eight (more preferably twenty, most preferably thirty) contiguous amino acids of SEQ ID NO:150, or a polynucleotide encoding a protein comprising a fragment of the amino acid sequence of SEQ ID NO:150 having biological activity, the fragment comprising the amino acid sequence from amino acid 39 to amino acid 48 of SEQ ID NO:150.

Other embodiments provide the gene corresponding to the cDNA sequence of SEQ ID NO:149, SEQ ID NO:148, and SEQ ID NO:151.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:148;
      (ab) SEQ ID NO:149;
      (ac) SEQ ID NO:151, but excluding the poly(A) tail at the 3′ end of SEQ ID NO:151; and
      (ad) the nucleotide sequence of the cDNA insert of clone D305_{—}2 deposited with the ATCC under accession number 98115;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:148;
      (bb) SEQ ID NO:149;
      (bc) SEQ ID NO:151, but excluding the poly(A) tail at the 3′ end of SEQ ID NO:151; and
      (bd) the nucleotide sequence of the cDNA insert of clone D305_{—}2 deposited with the ATCC under accession number 98115;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:148, SEQ ID NO:149, and SEQ ID NO:151, and extending contiguously from a nucleotide sequence corresponding to the 5′ end of SEQ ID NO:148 to a nucleotide sequence corresponding to the 3′ end of SEQ ID NO:151, but excluding the poly(A) tail at the 3′ end of SEQ ID NO:151. Also preferably the polynucleotide isolated according to the above process comprises a nucleotide sequence corresponding to the cDNA sequence of SEQ ID NO:149, and extending contiguously from a nucleotide sequence corresponding to the 5′ end of SEQ ID NO:149 to a nucleotide sequence corresponding to the 3′ end of SEQ ID NO:149. Also preferably the polynucleotide isolated according to the above process comprises a nucleotide sequence corresponding to the cDNA sequence of SEQ ID NO:149 from nucleotide 253 to nucleotide 519, and extending contiguously from a nucleotide sequence corresponding to the 5′ end of said sequence of SEQ ID NO:149 from nucleotide 253 to nucleotide 519, to a nucleotide sequence corresponding to the 3′ end of said sequence of SEQ ID NO:149 from nucleotide 253 to nucleotide 519. Also preferably the polynucleotide isolated according to the above process comprises a nucleotide sequence corresponding to the cDNA sequence of SEQ ID NO:149 from nucleotide 298 to nucleotide 519, and extending contiguously from a nucleotide sequence corresponding to the 5′ end of said sequence of SEQ ID NO:149 from nucleotide 298 to nucleotide 519, to a nucleotide sequence corresponding to the 3′ end of said sequence of SEQ ID NO:149 from nucleotide 298 to nucleotide 519.

In other embodiments, the present invention provides a composition comprising a protein, wherein said protein comprises an amino acid sequence selected from the group consisting of:
(a) the amino acid sequence of SEQ ID NO:150;
(b) a fragment of the amino acid sequence of SEQ ID NO:150, the fragment comprising eight contiguous amino acids of SEQ ID NO:150; and
(c) the amino acid sequence encoded by the cDNA insert of clone D305_{—}2 deposited with the ATCC under accession number 98115;
   the protein being substantially free from other mammalian proteins. Preferably such protein comprises the amino acid sequence of SEQ ID NO:150. In further preferred embodiments, the present invention provides a protein comprising a fragment of the amino acid sequence of SEQ ID NO:150 having biological activity, the fragment preferably comprising eight (more preferably twenty, most preferably thirty) contiguous amino acids of SEQ ID NO:150, or a protein comprising a fragment of the amino acid sequence of SEQ ID NO:150 having biological activity, the fragment comprising the amino acid sequence from amino acid 39 to amino acid 48 of SEQ ID NO:150.

In one embodiment, the present invention provides a composition comprising an isolated polynucleotide selected from the group consisting of:
(a) a polynucleotide comprising the nucleotide sequence of SEQ ID NO:152;
(b) a polynucleotide comprising the nucleotide sequence of SEQ ID NO:152 from nucleotide 521 to nucleotide 1306;
(c) a polynucleotide comprising the nucleotide sequence of SEQ ID NO:152 from nucleotide 851 to nucleotide 1306;
(d) a polynucleotide comprising the nucleotide sequence of SEQ ID NO:152 from nucleotide 443 to nucleotide 774;
(e) a polynucleotide comprising the nucleotide sequence of the full-length protein coding sequence of clone G55_{—}1 deposited with the ATCC under accession number 98115;
(f) a polynucleotide encoding the full-length protein encoded by the cDNA insert of clone G55_{—}1 deposited with the ATCC under accession number 98115;
(g) a polynucleotide comprising the nucleotide sequence of a mature protein coding sequence of clone G55-1 deposited with the ATCC under accession number 98115;
(h) a polynucleotide encoding a mature protein encoded by the cDNA insert of clone G55_{—}1 deposited with the ATCC under accession number 98115;
(i) a polynucleotide encoding a protein comprising the amino acid sequence of SEQ ID NO:153;
(j) a polynucleotide encoding a protein comprising a fragment of the amino acid sequence of SEQ ID NO:153 having biological activity, the fragment comprising eight contiguous amino acids of SEQ ID NO:153;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:152.

Preferably, such polynucleotide comprises the nucleotide sequence of SEQ ID NO:152 from nucleotide 521 to nucleotide 1306; the nucleotide sequence of SEQ ID NO:152 from nucleotide 851 to nucleotide 1306; the nucleotide sequence of SEQ ID NO:152 from nucleotide 443 to nucleotide 774; the nucleotide sequence of the full-length protein coding sequence of clone G55_{—}1 deposited with the ATCC under accession number 98115; or the nucleotide sequence of a mature protein coding sequence of clone G55_{—}1 deposited with the ATCC under accession number 98115. In other preferred embodiments, the polynucleotide encodes the full-length or a mature protein encoded by the cDNA insert of clone G55_{—}1 deposited with the ATCC under accession number 98115. In yet other preferred embodiments, the present invention provides a polynucleotide encoding a protein comprising the amino acid sequence of SEQ ID NO:153 from amino acid 1 to amino acid 32, or a polynucleotide encoding a protein comprising the amino acid sequence of SEQ ID NO:153 from amino acid 1 to amino acid 84. In further preferred embodiments, the present invention provides a polynucleotide encoding a protein comprising a fragment of the amino acid sequence of SEQ ID NO:153 having biological activity, the fragment preferably comprising eight (more preferably twenty, most preferably thirty) contiguous amino acids of SEQ ID NO:153, or a polynucleotide encoding a protein comprising a fragment of the amino acid sequence of SEQ ID NO:153 having biological activity, the fragment comprising the amino acid sequence from amino acid 126 to amino acid 135 of SEQ ID NO:153.

Other embodiments provide the gene corresponding to the cDNA sequence of SEQ ID NO:152.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:152, but excluding the poly(A) tail at the 3′ end of SEQ ID NO:152; and
      (ab) the nucleotide sequence of the cDNA insert of clone G55_{—}1 deposited with the ATCC under accession number 98115;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:152, but excluding the poly(A) tail at the 3′ end of SEQ ID NO:152; and
      (bb) the nucleotide sequence of the cDNA insert of clone G55_{—}1 deposited with the ATCC under accession number 98115;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:152, and extending contiguously from a nucleotide sequence corresponding to the 5′ end of SEQ ID NO:152 to a nucleotide sequence corresponding to the 3′ end of SEQ ID NO:152, but excluding the poly(A) tail at the 3′ end of SEQ ID NO:152. Also preferably the polynucleotide isolated according to the above process comprises a nucleotide sequence corresponding to the cDNA sequence of SEQ ID NO:152 from nucleotide 521 to nucleotide 1306, and extending contiguously from a nucleotide sequence corresponding to the 5′ end of said sequence of SEQ ID NO:152 from nucleotide 521 to nucleotide 1306, to a nucleotide sequence corresponding to the 3′ end of said sequence of SEQ ID NO:152 from nucleotide 521 to nucleotide 1306. Also preferably the polynucleotide isolated according to the above process comprises a nucleotide sequence corresponding to the cDNA sequence of SEQ ID NO:152 from nucleotide 851 to nucleotide 1306, and extending contiguously from a nucleotide sequence corresponding to the 5′ end of said sequence of SEQ ID NO:152 from nucleotide 851 to nucleotide 1306, to a nucleotide sequence corresponding to the 3′ end of said sequence of SEQ ID NO:152 from nucleotide 851 to nucleotide 1306. Also preferably the polynucleotide isolated according to the above process comprises a nucleotide sequence corresponding to the cDNA sequence of SEQ ID NO:152 from nucleotide 443 to nucleotide 774, and extending contiguously from a nucleotide sequence corresponding to the 5′ end of said sequence of SEQ ID NO:152 from nucleotide 443 to nucleotide 774, to a nucleotide sequence corresponding to the 3′ end of said sequence of SEQ ID NO:152 from nucleotide 443 to nucleotide 774.

In other embodiments, the present invention provides a composition comprising a protein, wherein said protein comprises an amino acid sequence selected from the group consisting of:
(a) the amino acid sequence of SEQ ID NO:153;
(b) the amino acid sequence of SEQ ID NO:153 from amino acid 1 to amino acid 32;
(c) a fragment of the amino acid sequence of SEQ ID NO:153, the fragment comprising eight contiguous amino acids of SEQ ID NO:153; and
(d) the amino acid sequence encoded by the cDNA insert of clone G55_{—}1 deposited with the ATCC under accession number 98115;
   the protein being substantially free from other mammalian proteins. Preferably such protein comprises the amino acid sequence of SEQ ID NO:153, the amino acid sequence of SEQ ID NO:153 from amino acid I to amino acid 32, or the amino acid sequence of SEQ ID NO:153 from amino acid 1 to amino acid 84. In further preferred embodiments, the present invention provides a protein comprising a fragment of the amino acid sequence of SEQ ID NO:153 having biological activity, the fragment preferably comprising eight (more preferably twenty, most preferably thirty) contiguous amino acids of SEQ ID NO:153, or a protein comprising a fragment of the amino acid sequence of SEQ ID NO:153 having biological activity, the fragment comprising the amino acid sequence from amino acid 126 to amino acid 135 of SEQ ID NO:153.

In one embodiment, the present invention provides a composition comprising an isolated polynucleotide selected from the group consisting of:
(a) a polynucleotide comprising the nucleotide sequence of SEQ ID NO:154;
(b) a polynucleotide comprising the nucleotide sequence of SEQ ID NO:154 from nucleotide 402 to nucleotide 533;
(c) a polynucleotide comprising the nucleotide sequence of SEQ ID NO:154 from nucleotide 447 to nucleotide 533;
(d) a polynucleotide comprising the nucleotide sequence of the full-length protein coding sequence of clone K39_{—}7 deposited with the ATCC under accession number 98115;
(e) a polynucleotide encoding the full-length protein encoded by the cDNA insert of clone K39_{—}7 deposited with the ATCC under accession number 98115;
(f) a polynucleotide comprising the nucleotide sequence of a mature protein coding sequence of clone K39_{—}7 deposited with the ATCC under accession number 98115;
(g) a polynucleotide encoding a mature protein encoded by the cDNA insert of clone K39_{—}7 deposited with the ATCC under accession number 98115;
(h) a polynucleotide encoding a protein comprising the amino acid sequence of SEQ ID NO:155;
(i) a polynucleotide encoding a protein comprising a fragment of the amino acid sequence of SEQ ID NO:155 having biological activity, the fragment comprising eight contiguous amino acids of SEQ ID NO:155;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:154.

Preferably, such polynucleotide comprises the nucleotide sequence of SEQ ID NO:154 from nucleotide 402 to nucleotide 533; the nucleotide sequence of SEQ ID NO:154 from nucleotide 447 to nucleotide 533; the nucleotide sequence of the full-length protein coding sequence of clone K39_{—}7 deposited with the ATCC under accession number 98115; or the nucleotide sequence of a mature protein coding sequence of clone K39_{—}7 deposited with the ATCC under accession number 98115. In other preferred embodiments, the polynucleotide encodes the full-length or a mature protein encoded by the cDNA insert of clone K39_{—}7 deposited with the ATCC under accession number 98115. In further preferred embodiments, the present invention provides a polynucleotide encoding a protein comprising a fragment of the amino acid sequence of SEQ ID NO:155 having biological activity, the fragment preferably comprising eight (more preferably twenty, most preferably thirty) contiguous amino acids of SEQ ID NO:155, or a polynucleotide encoding a protein comprising a fragment of the amino acid sequence of SEQ ID NO:155 having biological activity, the fragment comprising the amino acid sequence from amino acid 17 to amino acid 26 of SEQ ID NO:155.

Other embodiments provide the gene corresponding to the cDNA sequence of SEQ ID NO:154 and SEQ ID NO:156.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:154;
      (ab) SEQ ID NO:156, but excluding the poly(A) tail at the 3′ end of SEQ ID NO:156; and
      (ac) the nucleotide sequence of the cDNA insert of clone K39_{—}7 deposited with the ATCC under accession number 98115;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:154;
      (bb) SEQ ID NO:156, but excluding the poly(A) tail at the 3′ end of SEQ ID NO:156; and
      (bc) the nucleotide sequence of the cDNA insert of clone K39_{—}7 deposited with the ATCC under accession number 98115;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:154 and SEQ ID NO:156, and extending contiguously from a nucleotide sequence corresponding to the 5′ end of SEQ ID NO:154 to a nucleotide sequence corresponding to the 3′ end of SEQ ID NO:156, but excluding the poly(A) tail at the 3′ end of SEQ ID NO:156. Also preferably the polynucleotide isolated according to the above process comprises a nucleotide sequence corresponding to the cDNA sequence of SEQ ID NO:154, and extending contiguously from a nucleotide sequence corresponding to the 5′ end of SEQ ID NO:154 to a nucleotide sequence corresponding to the 3′ end of SEQ ID NO:154. Also preferably the polynucleotide isolated according to the above process comprises a nucleotide sequence corresponding to the cDNA sequence of SEQ ID NO:154 from nucleotide 402 to nucleotide 533, and extending contiguously from a nucleotide sequence corresponding to the 5′ end of said sequence of SEQ ID NO:154 from nucleotide 402 to nucleotide 533, to a nucleotide sequence corresponding to the 3′ end of said sequence of SEQ ID NO:154 from nucleotide 402 to nucleotide 533. Also preferably the polynucleotide isolated according to the above process comprises a nucleotide sequence corresponding to the cDNA sequence of SEQ ID NO:154 from nucleotide 447 to nucleotide 533, and extending contiguously from a nucleotide sequence corresponding to the 5′ end of said sequence of SEQ ID NO:154 from nucleotide 447 to nucleotide 533, to a nucleotide sequence corresponding to the 3′ end of said sequence of SEQ ID NO:154 from nucleotide 447 to nucleotide 533.

In other embodiments, the present invention provides a composition comprising a protein, wherein said protein comprises an amino acid sequence selected from the group consisting of:
(a) the amino acid sequence of SEQ ID NO:155;
(b) a fragment of the amino acid sequence of SEQ ID NO:155, the fragment comprising eight contiguous amino acids of SEQ ID NO:155; and
(c) the amino acid sequence encoded by the cDNA insert of clone K39_{—}7 deposited with the ATCC under accession number 98115;
   the protein being substantially free from other mammalian proteins. Preferably such protein comprises the amino acid sequence of SEQ ID NO:155. In further preferred embodiments, the present invention provides a protein comprising a fragment of the amino acid sequence of SEQ ID NO:155 having biological activity, the fragment preferably comprising eight (more preferably twenty, most preferably thirty) contiguous amino acids of SEQ ID NO:155, or a protein comprising a fragment of the amino acid sequence of SEQ ID NO:155 having biological activity, the fragment comprising the amino acid sequence from amino acid 17 to amino acid 26 of SEQ ID NO:155.

In one embodiment, the present invention provides a composition comprising an isolated polynucleotide selected from the group consisting of:
(a) a polynucleotide comprising the nucleotide sequence of SEQ ID NO:157;
(b) a polynucleotide comprising the nucleotide sequence of SEQ ID NO:157 from nucleotide 155 to nucleotide 583;
(c) a polynucleotide comprising the nucleotide sequence of SEQ ID NO:157 from nucleotide I to nucleotide 439;
(d) a polynucleotide comprising the nucleotide sequence of the full-length protein coding sequence of clone K330_{—}3 deposited with the ATCC under accession number 98115;
(e) a polynucleotide encoding the full-length protein encoded by the cDNA insert of clone K330_{—}3 deposited with the ATCC under accession number 98115;
(f) a polynucleotide comprising the nucleotide sequence of a mature protein coding sequence of clone K330_{—}3 deposited with the ATCC under accession number 98115;
(g) a polynucleotide encoding a mature protein encoded by the cDNA insert of clone K330_{—}3 deposited with the ATCC under accession number 98115;
(h) a polynucleotide encoding a protein comprising the amino acid sequence of SEQ ID NO:158;
(i) a polynucleotide encoding a protein comprising a fragment of the amino acid sequence of SEQ ID NO:158 having biological activity, the fragment comprising eight contiguous amino acids of SEQ ID NO:158;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:157.

Preferably, such polynucleotide comprises the nucleotide sequence of SEQ ID NO:157 from nucleotide 155 to nucleotide 583; the nucleotide sequence of SEQ ID NO:157 from nucleotide 1 to nucleotide 439; the nucleotide sequence of the full-length protein coding sequence of clone K330_{—}3 deposited with the ATCC under accession number 98115; or the nucleotide sequence of a mature protein coding sequence of clone K330_{—}3 deposited with the ATCC under accession number 98115. In other preferred embodiments, the polynucleotide encodes the full-length or a mature protein encoded by the cDNA insert of clone K330_{—}3 deposited with the ATCC under accession number 98115. In yet other preferred embodiments, the present invention provides a polynucleotide encoding a protein comprising the amino acid sequence of SEQ ID NO:158 from amino acid 1 to amino acid 95. In further preferred embodiments, the present invention provides a polynucleotide encoding a protein comprising a fragment of the ammo acid sequence of SEQ ID NO:158 having biological activity, the fragment preferably comprising eight (more preferably twenty, most preferably thirty) contiguous amino acids of SEQ ID NO:158, or a polynucleotide encoding a protein comprising a fragment of the amino acid sequence of SEQ ID NO:158 having biological activity, the fragment comprising the amino acid sequence from amino acid 66 to amino acid 75 of SEQ ID NO:158.

Other embodiments provide the gene corresponding to the cDNA sequence of SEQ ID NO:157.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:157, but excluding the poly(A) tail at the 3′ end of SEQ ID NO:157; and
      (ab) the nucleotide sequence of the cDNA insert of clone K330_{—}3 deposited with the ATCC under accession number 98115;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:157, but excluding the poly(A) tail at the 3′ end of SEQ ID NO:157; and
      (bb) the nucleotide sequence of the cDNA insert of clone K330_{—}3 deposited with the ATCC under accession number 98115;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:157, and extending contiguously from a nucleotide sequence corresponding to the 5′ end of SEQ ID NO:157 to a nucleotide sequence corresponding to the 3′ end of SEQ ID NO:157, but excluding the poly(A) tail at the 3′ end of SEQ ID NO:157. Also preferably the polynucleotide isolated according to the above process comprises a nucleotide sequence corresponding to the cDNA sequence of SEQ ID NO:157 from nucleotide 155 to nucleotide 583, and extending contiguously from a nucleotide sequence corresponding to the 5′ end of said sequence of SEQ ID NO:157 from nucleotide 155 to nucleotide 583, to a nucleotide sequence corresponding to the 3′ end of said sequence of SEQ ID NO:157 from nucleotide 155 to nucleotide 583. Also preferably the polynucleotide isolated according to the above process comprises a nucleotide sequence corresponding to the cDNA sequence of SEQ ID NO:157 from nucleotide 1 to nucleotide 439, and extending contiguously from a nucleotide sequence corresponding to the 5′ end of said sequence of SEQ ID NO:157 from nucleotide 1 to nucleotide 439, to a nucleotide sequence corresponding to the 3′ end of said sequence of SEQ ID NO:157 from nucleotide 1 to nucleotide 439.

In other embodiments, the present invention provides a composition comprising a protein, wherein said protein comprises an amino acid sequence selected from the group consisting of:
(a) the amino acid sequence of SEQ ID NO:158;
(b) the amino acid sequence of SEQ ID NO:158 from amino acid 1 to amino acid 95;
(c) a fragment of the amino acid sequence of SEQ ID NO:158, the fragment comprising eight contiguous amino acids of SEQ ID NO:158; and
(d) the amino acid sequence encoded by the cDNA insert of clone K330_{—}3 deposited with the ATCC under accession number 98115;
   the protein being substantially free from other mammalian proteins. Preferably such protein comprises the amino acid sequence of SEQ ID NO:158 or the amino acid sequence of SEQ ID NO:158 from amino acid 1 to amino acid 95. In further preferred embodiments, the present invention provides a protein comprising a fragment of the amino acid sequence of SEQ ID NO:158 having biological activity, the fragment preferably comprising eight (more preferably twenty, most preferably thirty) contiguous amino acids of SEQ ID NO:158, or a protein comprising a fragment of the amino acid sequence of SEQ ID NO:158 having biological activity, the fragment comprising the amino acid sequence from amino acid 66 to amino acid 75 of SEQ ID NO:158.

In one embodiment, the present invention provides a composition comprising an isolated polynucleotide selected from the group consisting of:
(a) a polynucleotide comprising the nucleotide sequence of SEQ ID NO:159;
(b) a polynucleotide comprising the nucleotide sequence of SEQ ID NO:159 from nucleotide 20 to nucleotide 988;
(c) a polynucleotide comprising the nucleotide sequence of SEQ ID NO:159 from nucleotide 701 to nucleotide 988;
(d) a polynucleotide comprising the nucleotide sequence of SEQ ID NO:159 from nucleotide 116 to nucleotide 309;
(e) a polynucleotide comprising the nucleotide sequence of the full-length protein coding sequence of clone K363_{—}3 deposited with the ATCC under accession number 98115;
(f) a polynucleotide encoding the full-length protein encoded by the cDNA insert of clone K363_{—}3 deposited with the ATCC under accession number 98115;
(g) a polynucleotide comprising the nucleotide sequence of a mature protein coding sequence of clone K363_{—}3 deposited with the ATCC under accession number 98115;
(h) a polynucleotide encoding a mature protein encoded by the cDNA insert of clone K363_{—}3 deposited with the ATCC under accession number 98115;
(i) a polynucleotide encoding a protein comprising the amino acid sequence of SEQ ID NO:160;
(j) a polynucleotide encoding a protein comprising a fragment of the amino acid sequence of SEQ ID NO:160 having biological activity, the fragment comprising eight contiguous amino acids of SEQ ID NO:160;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:159.

Preferably, such polynucleotide comprises the nucleotide sequence of SEQ ID NO:159 from nucleotide 20 to nucleotide 988; the nucleotide sequence of SEQ ID NO:159 from nucleotide 701 to nucleotide 988; the nucleotide sequence of SEQ ID NO:159 from nucleotide 116 to nucleotide 309; the nucleotide sequence of the full-length protein coding sequence of clone K363_{—}3 deposited with the ATCC under accession number 98115; or the nucleotide sequence of a mature protein coding sequence of clone K363_{—}3 deposited with the ATCC under accession number 98115. In other preferred embodiments, the polynucleotide encodes the full-length or a mature protein encoded by the cDNA insert of clone K363_{—}3 deposited with the ATCC under accession number 98115. In yet other preferred embodiments, the present invention provides a polynucleotide encoding a protein comprising the amino acid sequence of SEQ ID NO:160 from amino acid 33 to amino acid 96. In further preferred embodiments, the present invention provides a polynucleotide encoding a protein comprising a fragment of the amino acid sequence of SEQ ID NO:160 having biological activity, the fragment preferably comprising eight (more preferably twenty, most preferably thirty) contiguous amino acids of SEQ ID NO:160, or a polynucleotide encoding a protein comprising a fragment of the amino acid sequence of SEQ ID NO:160 having biological activity, the fragment comprising the amino acid sequence from amino acid 156 to amino acid 165 of SEQ ID NO:160.

Other embodiments provide the gene corresponding to the cDNA sequence of SEQ ID NO:159.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:159, but excluding the poly(A) tail at the 3′ end of SEQ ID NO:159; and
      (ab) the nucleotide sequence of the cDNA insert of clone K363_{—}3 deposited with the ATCC under accession number 98115;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:159, but excluding the poly(A) tail at the 3′ end of SEQ ID NO:159; and
      (bb) the nucleotide sequence of the cDNA insert of clone K363_{—}3 deposited with the ATCC under accession number 98115;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:159, and extending contiguously from a nucleotide sequence corresponding to the 5′ end of SEQ ID NO:159 to a nucleotide sequence corresponding to the 3′ end of SEQ ID NO:159, but excluding the poly(A) tail at the 3′ end of SEQ ID NO:159. Also preferably the polynucleotide isolated according to the above process comprises a nucleotide sequence corresponding to the cDNA sequence of SEQ ID NO:159 from nucleotide 20 to nucleotide 988, and extending contiguously from a nucleotide sequence corresponding to the 5′ end of said sequence of SEQ ID NO:159 from nucleotide 20 to nucleotide 988, to a nucleotide sequence corresponding to the 3′ end of said sequence of SEQ ID NO:159 from nucleotide 20 to nucleotide 988. Also preferably the polynucleotide isolated according to the above process comprises a nucleotide sequence corresponding to the cDNA sequence of SEQ ID NO:159 from nucleotide 701 to nucleotide 988, and extending contiguously from a nucleotide sequence corresponding to the 5′ end of said sequence of SEQ ID NO:159 from nucleotide 701 to nucleotide 988, to a nucleotide sequence corresponding to the 3′ end of said sequence of SEQ ID NO:159 from nucleotide 701 to nucleotide 988. Also preferably the polynucleotide isolated according to the above process comprises a nucleotide sequence corresponding to the cDNA sequence of SEQ ID NO:159 from nucleotide 116 to nucleotide 309, and extending contiguously from a nucleotide sequence corresponding to the 5′ end of said sequence of SEQ ID NO:159 from nucleotide 116 to nucleotide 309, to a nucleotide sequence corresponding to the 3′ end of said sequence of SEQ ID NO:159 from nucleotide 116 to nucleotide 309.

In other embodiments, the present invention provides a composition comprising a protein, wherein said protein comprises an amino acid sequence selected from the group consisting of:
(a) the amino acid sequence of SEQ ID NO:160;
(b) the amino acid sequence of SEQ ID NO:160 from amino acid 33 to amino acid 96;
(c) a fragment of the amino acid sequence of SEQ ID NO:160, the fragment comprising eight contiguous amino acids of SEQ ID NO:160; and
(d) the amino acid sequence encoded by the cDNA insert of clone K363_{—}3 deposited with the ATCC under accession number 98115;
   the protein being substantially free from other mammalian proteins. Preferably such protein comprises the amino acid sequence of SEQ ID NO:160 or the amino acid sequence of SEQ ID NO:160 from amino acid 33 to amino acid 96. In further preferred embodiments, the present invention provides a protein comprising a fragment of the amino acid sequence of SEQ ID NO:160 having biological activity, the fragment preferably comprising eight (more preferably twenty, most preferably thirty) contiguous amino acids of SEQ ID NO:160, or a protein comprising a fragment of the amino acid sequence of SEQ ID NO:160 having biological activity, the fragment comprising the amino acid sequence from amino acid 156 to amino acid 165 of SEQ ID NO:160.

In one embodiment, the present invention provides a composition comprising an isolated polynucleotide selected from the group consisting of:
(a) a polynucleotide comprising the nucleotide sequence of SEQ ID NO:161;
(b) a polynucleotide comprising the nucleotide sequence of SEQ ID NO:161 from nucleotide 342 to nucleotide 476;
(c) a polynucleotide comprising the nucleotide sequence of SEQ ID NO:161 from nucleotide 172 to nucleotide 467;
(d) a polynucleotide comprising the nucleotide sequence of the full-length protein coding sequence of clone K446_{—}3 deposited with the ATCC under accession number 98115;
(e) a polynucleotide encoding the full-length protein encoded by the cDNA insert of clone K446_{—}3 deposited with the ATCC under accession number 98115;
(f) a polynucleotide comprising the nucleotide sequence of a mature protein coding sequence of clone K446_{—}3 deposited with the ATCC under accession number 98115;
(g) a polynucleotide encoding a mature protein encoded by the cDNA insert of clone K446_{—}3 deposited with the ATCC under accession number 98115;
(h) a polynucleotide encoding a protein comprising the amino acid sequence of SEQ ID NO:162;
(i) a polynucleotide encoding a protein comprising a fragment of the amino acid sequence of SEQ ID NO:162 having biological activity, the fragment comprising eight contiguous amino acids of SEQ ID NO:162;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:161.

Preferably, such polynucleotide comprises the nucleotide sequence of SEQ ID NO:161 from nucleotide 342 to nucleotide 476; the nucleotide sequence of SEQ ID NO:161 from nucleotide 172 to nucleotide 467; the nucleotide sequence of the full-length protein coding sequence of clone K446_{—}3 deposited with the ATCC under accession number 98115; or the nucleotide sequence of a mature protein coding sequence of clone K446_{—}3 deposited with the ATCC under accession number 98115. In other preferred embodiments, the polynucleotide encodes the full-length or a mature protein encoded by the cDNA insert of clone K446_{—}3 deposited with the ATCC under accession number 98115. In yet other preferred embodiments, the present invention provides a polynucleotide encoding a protein comprising the amino acid sequence of SEQ ID NO:162 from amino acid 1 to amino acid 42. In further preferred embodiments, the present invention provides a polynucleotide encoding a protein comprising a fragment of the amino acid sequence of SEQ ID NO:162 having biological activity, the fragment preferably comprising eight (more preferably twenty, most preferably thirty) contiguous amino acids of SEQ ID NO:162, or a polynucleotide encoding a protein comprising a fragment of the amino acid sequence of SEQ ID NO:162 having biological activity, the fragment comprising the amino acid sequence from amino acid 17 to amino acid 26 of SEQ ID NO:162.

Other embodiments provide the gene corresponding to the cDNA sequence of SEQ ID NO:161.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:161, but excluding the poly(A) tail at the 3′ end of SEQ ID NO:161; and
      (ab) the nucleotide sequence of the cDNA insert of clone K446_{—}3 deposited with the ATCC under accession number 98115;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:161, but excluding the poly(A) tail at the 3′ end of SEQ ID NO:161; and
      (bb) the nucleotide sequence of the cDNA insert of clone K446_{—}3 deposited with the ATCC under accession number 98115;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:161, and extending contiguously from a nucleotide sequence corresponding to the 5′ end of SEQ ID NO:161 to a nucleotide sequence corresponding to the 3′ end of SEQ ID NO:161, but excluding the poly(A) tail at the 3′ end of SEQ ID NO:161. Also preferably the polynucleotide isolated according to the above process comprises a nucleotide sequence corresponding to the cDNA sequence of SEQ ID NO:161 from nucleotide 342 to nucleotide 476, and extending contiguously from a nucleotide sequence corresponding to the 5′ end of said sequence of SEQ ID NO:161 from nucleotide 342 to nucleotide 476, to a nucleotide sequence corresponding to the 3′ end of said sequence of SEQ ID NO:161 from nucleotide 342 to nucleotide 476. Also preferably the polynucleotide isolated according to the above process comprises a nucleotide sequence corresponding to the cDNA sequence of SEQ ID NO:161 from nucleotide 172 to nucleotide 467, and extending contiguously from a nucleotide sequence corresponding to the 5′ end of said sequence of SEQ ID NO:161 from nucleotide 172 to nucleotide 467, to a nucleotide sequence corresponding to the 3′ end of said sequence of SEQ ID NO:161 from nucleotide 172 to nucleotide 467.

In other embodiments, the present invention provides a composition comprising a protein, wherein said protein comprises an amino acid sequence selected from the group consisting of:
(a) the amino acid sequence of SEQ ID NO:162;
(b) the amino acid sequence of SEQ ID NO:162 from amino acid 1 to amino acid 42;
(c) a fragment of the amino acid sequence of SEQ ID NO:162, the fragment comprising eight contiguous amino acids of SEQ ID NO:162; and
(d) the amino acid sequence encoded by the cDNA insert of clone K446_{—}3 deposited with the ATCC under accession number 98115;
   the protein being substantially free from other mammalian proteins. Preferably such protein comprises the amino acid sequence of SEQ ID NO:162 or the amino acid sequence of SEQ ID NO:162 from amino acid 1 to amino acid 42. In further preferred embodiments, the present invention provides a protein comprising a fragment of the amino acid sequence of SEQ ID NO:162 having biological activity, the fragment preferably comprising eight (more preferably twenty, most preferably thirty) contiguous amino acids of SEQ ID NO:162, or a protein comprising a fragment of the amino acid sequence of SEQ ID NO:162 having biological activity, the fragment comprising the amino acid sequence from amino acid 17 to amino acid 26 of SEQ ID NO:162.

In one embodiment, the present invention provides a composition comprising an isolated polynucleotide selected from the group consisting of:
(a) a polynucleotide comprising the nucleotide sequence of SEQ ID NO:163;
(b) a polynucleotide comprising the nucleotide sequence of SEQ ID NO:163 from nucleotide 214 to nucleotide 387;
(c) a polynucleotide comprising the nucleotide sequence of SEQ ID NO:163 from nucleotide 199 to nucleotide 295;
(d) a polynucleotide comprising the nucleotide sequence of the full-length protein coding sequence of clone K464_{—}4 deposited with the ATCC under accession number 98115;
(e) a polynucleotide encoding the full-length protein encoded by the cDNA insert of clone K464_{—}4 deposited with the ATCC under accession number 98115;
(f) a polynucleotide comprising the nucleotide sequence of a mature protein coding sequence of clone K464_{—}4 deposited with the ATCC under accession number 98115;
(g) a polynucleotide encoding a mature protein encoded by the cDNA insert of clone K464_{—}4 deposited with the ATCC under accession number 98115;
(h) a polynucleotide encoding a protein comprising the amino acid sequence of SEQ ID NO:164;
(i) a polynucleotide encoding a protein comprising a fragment of the amino acid sequence of SEQ ID NO:164 having biological activity, the fragment comprising eight contiguous amino acids of SEQ ID NO:164;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:163.

Preferably, such polynucleotide comprises the nucleotide sequence of SEQ ID NO:163 from nucleotide 214 to nucleotide 387; the nucleotide sequence of SEQ ID NO:163 from nucleotide 199 to nucleotide 295; the nucleotide sequence of the full-length protein coding sequence of clone K464_{—}4 deposited with the ATCC under accession number 98115; or the nucleotide sequence of a mature protein coding sequence of clone K464_{—}4 deposited with the ATCC under accession number 98115. In other preferred embodiments, the polynucleotide encodes the full-length or a mature protein encoded by the cDNA insert of clone K464_{—}4 deposited with the ATCC under accession number 98115. In yet other preferred embodiments, the present invention provides a polynucleotide encoding a protein comprising the amino acid sequence of SEQ ID NO:164 from amino acid 1 to amino acid 27. In further preferred embodiments, the present invention provides a polynucleotide encoding a protein comprising a fragment of the amino acid sequence of SEQ ID NO:164 having biological activity, the fragment preferably comprising eight (more preferably twenty, most preferably thirty) contiguous amino acids of SEQ ID NO:164, or a polynucleotide encoding a protein comprising a fragment of the amino acid sequence of SEQ ID NO:164 having biological activity, the fragment comprising the amino acid sequence from amino acid 24 to amino acid 33 of SEQ ID NO:164.

Other embodiments provide the gene corresponding to the cDNA sequence of SEQ ID NO:163.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:163, but excluding the poly(A) tail at the 3′ end of SEQ ID NO:163; and
      (ab) the nucleotide sequence of the cDNA insert of clone K464_{—}4 deposited with the ATCC under accession number 98115;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:163, but excluding the poly(A) tail at the 3′ end of SEQ ID NO:163; and
      (bb) the nucleotide sequence of the cDNA insert of clone K464_{—}4 deposited with the ATCC under accession number 98115;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:163, and extending contiguously from a nucleotide sequence corresponding to the 5′ end of SEQ ID NO:163 to a nucleotide sequence corresponding to the 3′ end of SEQ ID NO:163, but excluding the poly(A) tail at the 3′ end of SEQ ID NO:163. Also preferably the polynucleotide isolated according to the above process comprises a nucleotide sequence corresponding to the cDNA sequence of SEQ ID NO:163 from nucleotide 214 to nucleotide 387, and extending contiguously from a nucleotide sequence corresponding to the 5′ end of said sequence of SEQ ID NO:163 from nucleotide 214 to nucleotide 387, to a nucleotide sequence corresponding to the 3′ end of said sequence of SEQ ID NO:163 from nucleotide 214 to nucleotide 387. Also preferably the polynucleotide isolated according to the above process comprises a nucleotide sequence corresponding to the cDNA sequence of SEQ ID NO:163 from nucleotide 199 to nucleotide 295, and extending contiguously from a nucleotide sequence corresponding to the 5 end of said sequence of SEQ ID NO:163 from nucleotide 199 to nucleotide 295, to a nucleotide sequence corresponding to the 3′ end of said sequence of SEQ ID NO:163 from nucleotide 199 to nucleotide 295.

In other embodiments, the present invention provides a composition comprising a protein, wherein said protein comprises an amino acid sequence selected from the group consisting of:
(a) the amino acid sequence of SEQ ID NO:164;
(b) the amino acid sequence of SEQ ID NO:164 from amino acid 1 to amino acid 27;
(c) a fragment of the amino acid sequence of SEQ ID NO:164, the fragment comprising eight contiguous amino acids of SEQ ID NO:164; and
(d) the amino acid sequence encoded by the cDNA insert of clone K464_{—}4 deposited with the ATCC under accession number 98115;
   the protein being substantially free from other mammalian proteins. Preferably such protein comprises the amino acid sequence of SEQ ID NO:164 or the amino acid sequence of SEQ ID NO:164 from amino acid I to amino acid 27. In further preferred embodiments, the present invention provides a protein comprising a fragment of the amino acid sequence of SEQ ID NO:164 having biological activity, the fragment preferably comprising eight (more preferably twenty, most preferably thirty) contiguous amino acids of SEQ ID NO:164, or a protein comprising a fragment of the amino acid sequence of SEQ ID NO:164 having biological activity, the fragment comprising the amino acid sequence from amino acid 24 to amino acid 33 of SEQ ID NO:164.

In one embodiment, the present invention provides a composition comprising an isolated polynucleotide selected from the group consisting of:
(a) a polynucleotide comprising the nucleotide sequence of SEQ ID NO:165;
(b) a polynucleotide comprising the nucleotide sequence of SEQ ID NO:165 from nucleotide 218 to nucleotide 1159;
(c) a polynucleotide comprising the nucleotide sequence of SEQ ID NO:165 from nucleotide 806 to nucleotide 1159;
(d) a polynucleotide comprising the nucleotide sequence of SEQ ID NO:165 from nucleotide 217 to nucleotide 517;
(e) a polynucleotide comprising the nucleotide sequence of the full-length protein coding sequence of clone K483_{—}1 deposited with the ATCC under accession number 98115;
(f) a polynucleotide encoding the full-length protein encoded by the cDNA insert of clone K483_{—}1 deposited with the ATCC under accession number 98115;
(g) a polynucleotide comprising the nucleotide sequence of a mature protein coding sequence of clone K483_{—}1 deposited with the ATCC under accession number 98115;
(h) a polynucleotide encoding a mature protein encoded by the cDNA insert of clone K483_{—}1 deposited with the ATCC under accession number 98115;
(i) a polynucleotide encoding a protein comprising the amino acid sequence of SEQ ID NO:166;
(j) a polynucleotide encoding a protein comprising a fragment of the amino acid sequence of SEQ ID NO:166 having biological activity, the fragment comprising eight contiguous amino acids of SEQ ID NO:166;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:165.

Preferably, such polynucleotide comprises the nucleotide sequence of SEQ ID NO:165 from nucleotide 218 to nucleotide 1159; the nucleotide sequence of SEQ ID NO:165 from nucleotide 806 to nucleotide 1159; the nucleotide sequence of SEQ ID NO:165 from nucleotide 217 to nucleotide 517; the nucleotide sequence of the full-length protein coding sequence of clone K483_{—}1 deposited with the ATCC under accession number 98115; or the nucleotide sequence of a mature protein coding sequence of clone K483_{—}1 deposited with the ATCC under accession number 98115. In other preferred embodiments, the polynucleotide encodes the full-length or a mature protein encoded by the cDNA insert of clone K483_{—}1 deposited with the ATCC under accession number 98115. In yet other preferred embodiments, the present invention provides a polynucleotide encoding a protein comprising the amino acid sequence of SEQ ID NO:166 from amino acid 1 to amino acid 100. In further preferred embodiments, the present invention provides a polynucleotide encoding a protein comprising a fragment of the amino acid sequence of SEQ ID NO:166 having biological activity, the fragment preferably comprising eight (more preferably twenty, most preferably thirty) contiguous amino acids of SEQ ID NO:166, or a polynucleotide encoding a protein comprising a fragment of the amino acid sequence of SEQ ID NO:166 having biological activity, the fragment comprising the amino acid sequence from amino acid 152 to amino acid 161 of SEQ ID NO:166.

Other embodiments provide the gene corresponding to the cDNA sequence of SEQ ID NO:165.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:165, but excluding the poly(A) tail at the 3′ end of SEQ ID NO:165; and
      (ab) the nucleotide sequence of the cDNA insert of clone K483_{—}1 deposited with the ATCC under accession number 98115;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:165, but excluding the poly(A) tail at the 3′ end of SEQ ID NO:165; and
      (bb) the nucleotide sequence of the cDNA insert of clone K483_{—}1 deposited with the ATCC under accession number 98115;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:165, and extending contiguously from a nucleotide sequence corresponding to the 5′ end of SEQ ID NO:165 to a nucleotide sequence corresponding to the 3′ end of SEQ ID NO:165, but excluding the poly(A) tail at the 3′ end of SEQ ID NO:165. Also preferably the polynucleotide isolated according to the above process comprises a nucleotide sequence corresponding to the cDNA sequence of SEQ ID NO:165 from nucleotide 218 to nucleotide 1159, and extending contiguously from a nucleotide sequence corresponding to the 5′ end of said sequence of SEQ ID NO:165 from nucleotide 218 to nucleotide 1159, to a nucleotide sequence corresponding to the 3′ end of said sequence of SEQ ID NO:165 from nucleotide 218 to nucleotide 1159. Also preferably the polynucleotide isolated according to the above process comprises a nucleotide sequence corresponding to the cDNA sequence of SEQ ID NO:165 from nucleotide 806 to nucleotide 1159, and extending contiguously from a nucleotide sequence corresponding to the 5′ end of said sequence of SEQ ID NO:165 from nucleotide 806 to nucleotide 1159, to a nucleotide sequence corresponding to the 3′ end of said sequence of SEQ ID NO:165 from nucleotide 806 to nucleotide 1159. Also preferably the polynucleotide isolated according to the above process comprises a nucleotide sequence corresponding to the cDNA sequence of SEQ ID NO:165 from nucleotide 217 to nucleotide 517, and extending contiguously from a nucleotide sequence corresponding to the 5′ end of said sequence of SEQ ID NO:165 from nucleotide 217 to nucleotide 517, to a nucleotide sequence corresponding to the 3′ end of said sequence of SEQ ID NO:165 from nucleotide 217 to nucleotide 517.

In other embodiments, the present invention provides a composition comprising a protein, wherein said protein comprises an amino add sequence selected from the group consisting of:
(a) the amino acid sequence of SEQ ID NO:166;
(b) the amino acid sequence of SEQ ID NO:166 from amino acid 1 to amino acid 100;
(c) a fragment of the amino acid sequence of SEQ ID NO:166, the fragment comprising eight contiguous amino acids of SEQ ID NO:166; and
(d) the amino acid sequence encoded by the cDNA insert of clone K483_{—}1 deposited with the ATCC under accession number 98115;
   the protein being substantially free from other mammalian proteins. Preferably such protein comprises the amino acid sequence of SEQ ID NO:166 or the amino acid sequence of SEQ ID NO:166 from amino acid 1 to amino acid 100. In further preferred embodiments, the present invention provides a protein comprising a fragment of the amino acid sequence of SEQ ID NO:166 having biological activity, the fragment preferably comprising eight (more preferably twenty, most preferably thirty) contiguous amino acids of SEQ ID NO:166, or a protein comprising a fragment of the amino acid sequence of SEQ ID NO:166 having biological activity, the fragment comprising the amino acid sequence from amino acid 152 to amino acid 161 of SEQ ID NO:166.

In one embodiment, the present invention provides a composition comprising an isolated polynucleotide selected from the group consisting of:
(a) a polynucleotide comprising the nucleotide sequence of SEQ ID NO:167;
(b) a polynucleotide comprising the nucleotide sequence of SEQ ID NO:167 from nucleotide 15 to nucleotide 461;
(c) a polynucleotide comprising the nucleotide sequence of SEQ ID NO:167 from nucleotide 63 to nucleotide 461;
(d) a polynucleotide comprising the nucleotide sequence of SEQ ID NO:167 from nucleotide 1 to nucleotide 217;
(e) a polynucleotide comprising the nucleotide sequence of the full-length protein coding sequence of clone L69_{—}3 deposited with the ATCC under accession number 98115;
(f) a polynucleotide encoding the full-length protein encoded by the cDNA insert of clone L69_{—}3 deposited with the ATCC under accession number 98115;
(g) a polynucleotide comprising the nucleotide sequence of a mature protein coding sequence of clone L69_{—}3 deposited with the ATCC under accession number 98115;
(h) a polynucleotide encoding a mature protein encoded by the cDNA insert of clone L69_{—}3 deposited with the ATCC under accession number 98115;
(i) a polynucleotide encoding a protein comprising the amino acid sequence of SEQ ID NO:168;
(j) a polynucleotide encoding a protein comprising a fragment of the amino acid sequence of SEQ ID NO:168 having biological activity, the fragment comprising eight contiguous amino acids of SEQ ID NO:168;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:167.

Preferably, such polynucleotide comprises the nucleotide sequence of SEQ ID NO:167 from nucleotide 15 to nucleotide 461; the nucleotide sequence of SEQ ID NO:167 from nucleotide 63 to nucleotide 461; the nucleotide sequence of SEQ ID NO:167 from nucleotide 1 to nucleotide 217; the nucleotide sequence of the full-length protein coding sequence of clone L69_{—}3 deposited with the ATCC under accession number 98115; or the nucleotide sequence of a mature protein coding sequence of clone L69_{—}3 deposited with the ATCC under accession number 98115. In other preferred embodiments, the polynucleotide encodes the full-length or a mature protein encoded by the cDNA insert of clone L69_{—}3 deposited with the ATCC under accession number 98115. In yet other preferred embodiments, the present invention provides a polynucleotide encoding a protein comprising the amino acid sequence of SEQ ID NO:168 from amino acid 1 to amino acid 67. In further preferred embodiments, the present invention provides a polynucleotide encoding a protein comprising a fragment of the amino acid sequence of SEQ ID NO:168 having biological activity, the fragment preferably comprising eight (more preferably twenty, most preferably thirty) contiguous amino acids of SEQ ID NO:168, or a polynucleotide encoding a protein comprising a fragment of the amino acid sequence of SEQ ID NO:168 having biological activity, the fragment comprising the amino add sequence from amino add 69 to amino acid 78 of SEQ ID NO:168.

Other embodiments provide the gene corresponding to the cDNA sequence of SEQ ID NO:167.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:167, but excluding the poly(A) tail at the 3′ end of SEQ ID NO:167; and
      (ab) the nucleotide sequence of the cDNA insert of clone L69_{—}3 deposited with the ATCC under accession number 98115;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:167, but excluding the poly(A) tail at the 3′ end of SEQ ID NO:167; and
      (bb) the nucleotide sequence of the cDNA insert of clone L69_{—}3 deposited with the ATCC under accession number 98115;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:167, and extending contiguously from a nucleotide sequence corresponding to the 5′ end of SEQ ID NO:167 to a nucleotide sequence corresponding to the 3′ end of SEQ ID NO:167, but excluding the poly(A) tail at the 3′ end of SEQ ID NO:167. Also preferably the polynucleotide isolated according to the above process comprises a nucleotide sequence corresponding to the cDNA sequence of SEQ ID NO:167 from nucleotide 15 to nucleotide 461, and extending contiguously from a nucleotide sequence corresponding to the 5′ end of said sequence of SEQ ID NO:167 from nucleotide 15 to nucleotide 461, to a nucleotide sequence corresponding to the 3′ end of said sequence of SEQ ID NO:167 from nucleotide 15 to nucleotide 461. Also preferably the polynucleotide isolated according to the above process comprises a nucleotide sequence corresponding to the cDNA sequence of SEQ ID NO:167 from nucleotide 63 to nucleotide 461, and extending contiguously from a nucleotide sequence corresponding to the 5′ end of said sequence of SEQ ID NO:167 from nucleotide 63 to nucleotide 461, to a nucleotide sequence corresponding to the 3′ end of said sequence of SEQ ID NO:167 from nucleotide 63 to nucleotide 461. Also preferably the polynucleotide isolated according to the above process comprises a nucleotide sequence corresponding to the cDNA sequence of SEQ ID NO:167 from nucleotide 1 to nucleotide 217, and extending contiguously from a nucleotide sequence corresponding to the 5′ end of said sequence of SEQ ID NO:167 from nucleotide 1 to nucleotide 217, to a nucleotide sequence corresponding to the 3′ end of said sequence of SEQ ID NO:167 from nucleotide 1 to nucleotide 217.

In other embodiments, the present invention provides a composition comprising a protein, wherein said protein comprises an amino acid sequence selected from the group consisting of:
(a) the amino acid sequence of SEQ ID NO:168;
(b) the amino acid sequence of SEQ ID NO:168 from amino acid 1 to amino acid 67;
(c) a fragment of the amino acid sequence of SEQ ID NO:168, the fragment comprising eight contiguous amino acids of SEQ ID NO:168; and
(d) the amino acid sequence encoded by the cDNA insert of clone L69_{—}3 deposited with the ATCC under accession number 98115;
   the protein being substantially free from other mammalian proteins. Preferably such protein comprises the amino acid sequence of SEQ ID NO:168 or the amino acid sequence of SEQ ID NO:168 from amino acid 1 to amino acid 67. In further preferred embodiments, the present invention provides a protein comprising a fragment of the amino acid sequence of SEQ ID NO:168 having biological activity, the fragment preferably comprising eight (more preferably twenty, most preferably thirty) contiguous amino acids of SEQ ID NO:168, or a protein comprising a fragment of the amino acid sequence of SEQ ID NO:168 having biological activity, the fragment comprising the amino acid sequence from amino acid 69 to amino acid 78 of SEQ ID NO:168.

In one embodiment, the present invention provides a composition comprising an isolated polynucleotide selected from the group consisting of:
(a) a polynucleotide comprising the nucleotide sequence of SEQ ID NO:169;
(b) a polynucleotide comprising the nucleotide sequence of SEQ ID NO:169 from nucleotide 2322 to nucleotide 2669;
(c) a polynucleotide comprising the nucleotide sequence of SEQ ID NO:169 from nucleotide 1253 to nucleotide 1573;
(d) a polynucleotide comprising the nucleotide sequence of the full-length protein coding sequence of clone BG511_{—}30 deposited with the ATCC under accession number 98117;
(e) a polynucleotide encoding the full-length protein encoded by the cDNA insert of clone BG511_{—}30 deposited with the ATCC under accession number 98117;
(f) a polynucleotide comprising the nucleotide sequence of a mature protein coding sequence of clone BG511_{—}30 deposited with the ATCC under accession number 98117;
(g) a polynucleotide encoding a mature protein encoded by the cDNA insert of clone BG511_{—}30 deposited with the ATCC under accession number 98117;
(h) a polynucleotide encoding a protein comprising the amino acid sequence of SEQ ID NO:170;
(i) a polynucleotide encoding a protein comprising a fragment of the amino acid sequence of SEQ ID NO:170 having biological activity, the fragment comprising eight contiguous amino acids of SEQ ID NO:170;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:169.

Preferably, such polynucleotide comprises the nucleotide sequence of SEQ ID NO:169 from nucleotide 2322 to nucleotide 2669; the nucleotide sequence of SEQ ID NO:169 from nucleotide 1253 to nucleotide 1573; the nucleotide sequence of the full-length protein coding sequence of clone BG511_{—}30 deposited with the ATCC under accession number 98117; or the nucleotide sequence of a mature protein coding sequence of clone BG511_{—}30 deposited with the ATCC under accession number 98117. In other preferred embodiments, the polynucleotide encodes the full-length or a mature protein encoded by the cDNA insert of clone BG511_{—}30 deposited with the ATCC under accession number 98117. In further preferred embodiments, the present invention provides a polynucleotide encoding a protein comprising a fragment of the amino acid sequence of SEQ ID NO:170 having biological activity, the fragment preferably comprising eight (more preferably twenty, most preferably thirty) contiguous amino acids of SEQ ID NO:170, or a polynucleotide encoding a protein comprising a fragment of the amino acid sequence of SEQ ID NO:170 having biological activity, the fragment comprising the amino acid sequence from amino acid 53 to amino acid 62 of SEQ ID NO:170.

Other embodiments provide the gene corresponding to the cDNA sequence of SEQ ID NO:169.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:169, but excluding the poly(A) tail at the 3′ end of SEQ ID NO:169; and
      (ab) the nucleotide sequence of the cDNA insert of clone BG511_{—}30 deposited with the ATCC under accession number 98117;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:169, but excluding the poly(A) tail at the 3′ end of SEQ ID NO:169; and
      (bb) the nucleotide sequence of the cDNA insert of clone BG511_{—}30 deposited with the ATCC under accession number 98117;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:169, and extending contiguously from a nucleotide sequence corresponding to the 5′ end of SEQ ID NO:169 to a nucleotide sequence corresponding to the 3′ end of SEQ ID NO:169, but excluding the poly(A) tail at the 3′ end of SEQ ID NO:169. Also preferably the polynucleotide isolated according to the above process comprises a nucleotide sequence corresponding to the cDNA sequence of SEQ ID NO:169 from nucleotide 2322 to nucleotide 2669, and extending contiguously from a nucleotide sequence corresponding to the 5′ end of said sequence of SEQ ID NO:169 from nucleotide 2322 to nucleotide 2669, to a nucleotide sequence corresponding to the 3′ end of said sequence of SEQ ID NO:169 from nucleotide 2322 to nucleotide 2669. Also preferably the polynucleotide isolated according to the above process comprises a nucleotide sequence corresponding to the cDNA sequence of SEQ ID NO:169 from nucleotide 1253 to nucleotide 1573, and extending contiguously from a nucleotide sequence corresponding to the 5′ end of said sequence of SEQ ID NO:169 from nucleotide 1253 to nucleotide 1573, to a nucleotide sequence corresponding to the 3′ end of said sequence of SEQ ID NO:169 from nucleotide 1253 to nucleotide 1573.

In other embodiments, the present invention provides a composition comprising a protein, wherein said protein comprises an amino acid sequence selected from the group consisting of:
(a) the amino acid sequence of SEQ ID NO:170;
(b) a fragment of the amino acid sequence of SEQ ID NO:170, the fragment comprising eight contiguous amino acids of SEQ ID NO:170; and
(c) the amino acid sequence encoded by the cDNA insert of clone BG511_{—}30 deposited with the ATCC under accession number 98117;
   the protein being substantially free from other mammalian proteins. Preferably such protein comprises the amino acid sequence of SEQ ID NO:170. In further preferred embodiments, the present invention provides a protein comprising a fragment of the amino acid sequence of SEQ ID NO:170 having biological activity, the fragment preferably comprising eight (more preferably twenty, most preferably thirty) contiguous amino acids of SEQ ID NO:170, or a protein comprising a fragment of the amino acid sequence of SEQ ID NO:170 having biological activity, the fragment comprising the amino acid sequence from amino acid 53 to amino acid 62 of SEQ ID NO:170.

In one embodiment, the present invention provides a composition comprising an isolated polynucleotide selected from the group consisting of:
(a) a polynucleotide comprising the nucleotide sequence of SEQ ID NO:171;
(b) a polynucleotide comprising the nucleotide sequence of SEQ ID NO:171 from nucleotide 220 to nucleotide 378;
(c) a polynucleotide comprising the nucleotide sequence of SEQ ID NO:171 from nucleotide 356 to nucleotide 708;
(d) a polynucleotide comprising the nucleotide sequence of the full-length protein coding sequence of clone BL15_{—}12 deposited with the ATCC under accession number 98154;
(e) a polynucleotide encoding the full-length protein encoded by the cDNA insert of clone BL15_{—}12 deposited with the ATCC under accession number 98154;
(f) a polynucleotide comprising the nucleotide sequence of a mature protein coding sequence of clone BL15_{—}12 deposited with the ATCC under accession number 98154;
(g) a polynucleotide encoding a mature protein encoded by the cDNA insert of clone BL15_{—}12 deposited with the ATCC under accession number 98154;
(h) a polynucleotide encoding a protein comprising the amino acid sequence of SEQ ID NO:172;
(i) a polynucleotide encoding a protein comprising a fragment of the amino acid sequence of SEQ ID NO:172 having biological activity, the fragment comprising eight contiguous amino acids of SEQ ID NO:172;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:171.

Preferably, such polynucleotide comprises the nucleotide sequence of SEQ ID NO:171 from nucleotide 220 to nucleotide 378; the nucleotide sequence of SEQ ID NO:171 from nucleotide 356 to nucleotide 708; the nucleotide sequence of the full-length protein coding sequence of clone BL15_{—}12 deposited with the ATCC under accession number 98154; or the nucleotide sequence of a mature protein coding sequence of clone BL15_{—}12 deposited with the ATCC under accession number 98154. In other preferred embodiments, the polynucleotide encodes the full-length or a mature protein encoded by the cDNA insert of clone BL15_{—}12 deposited with the ATCC under accession number 98154. In further preferred embodiments, the present invention provides a polynucleotide encoding a protein comprising a fragment of the amino acid sequence of SEQ ID NO:172 having biological activity, the fragment preferably comprising eight (more preferably twenty, most preferably thirty) contiguous amino acids of SEQ ID NO:172, or a polynucleotide encoding a protein comprising a fragment of the amino acid sequence of SEQ ID NO:172 having biological activity, the fragment comprising the amino acid sequence from amino acid 21 to amino acid 30 of SEQ ID NO:172.

Other embodiments provide the gene corresponding to the cDNA sequence of SEQ ID NO:171.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:171, but excluding the poly(A) tail at the 3′ end of SEQ ID NO:171; and
      (ab) the nucleotide sequence of the cDNA insert of clone BL15_{—}12 deposited with the ATCC under accession number 98154;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:171, but excluding the poly(A) tail at the 3′ end of SEQ ID NO:171; and
      (bb) the nucleotide sequence of the cDNA insert of clone BL15_{—}12 deposited with the ATCC under accession number 98154;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:171, and extending contiguously from a nucleotide sequence corresponding to the 5′ end of SEQ ID NO:171 to a nucleotide sequence corresponding to the 3′ end of SEQ ID NO:171, but excluding the poly(A) tail at the 3′ end of SEQ ID NO:171. Also preferably the polynucleotide isolated according to the above process comprises a nucleotide sequence corresponding to the cDNA sequence of SEQ ID NO:171 from nucleotide 220 to nucleotide 378, and extending contiguously from a nucleotide sequence corresponding to the 5′ end of said sequence of SEQ ID NO:171 from nucleotide 220 to nucleotide 378, to a nucleotide sequence corresponding to the 3′ end of said sequence of SEQ ID NO:171 from nucleotide 220 to nucleotide 378. Also preferably the polynucleotide isolated according to the above process comprises a nucleotide sequence corresponding to the cDNA sequence of SEQ ID NO:171 from nucleotide 356 to nucleotide 708, and extending contiguously from a nucleotide sequence corresponding to the 5′ end of said sequence of SEQ ID NO:171 from nucleotide 356 to nucleotide 708, to a nucleotide sequence corresponding to the 3′ end of said sequence of SEQ ID NO:171 from nucleotide 356 to nucleotide 708.

In other embodiments, the present invention provides a composition comprising a protein, wherein said protein comprises an amino acid sequence selected from the group consisting of:
(a) the amino acid sequence of SEQ ID NO:172;
(b) a fragment of the amino acid sequence of SEQ ID NO:172, the fragment comprising eight contiguous amino acids of SEQ ID NO:172; and
(c) the amino acid sequence encoded by the cDNA insert of clone BL15_{—}12 deposited with the ATCC under accession number 98154;
   the protein being substantially free from other mammalian proteins. Preferably such protein comprises the amino acid sequence of SEQ ID NO:172. In further preferred embodiments, the present invention provides a protein comprising a fragment of the amino acid sequence of SEQ ID NO:172 having biological activity, the fragment preferably comprising eight (more preferably twenty, most preferably thirty) contiguous amino acids of SEQ ID NO:172, or a protein comprising a fragment of the amino acid sequence of SEQ ID NO:172 having biological activity, the fragment comprising the amino acid sequence from amino acid 21 to amino acid 30 of SEQ ID NO:172.

In one embodiment, the present invention provides a composition comprising an isolated polynucleotide selected from the group consisting of:
(a) a polynucleotide comprising the nucleotide sequence of SEQ ID NO:173;
(b) a polynucleotide comprising the nucleotide sequence of SEQ ID NO:173 from nucleotide 257 to nucleotide 1366;
(c) a polynucleotide comprising the nucleotide sequence of SEQ ID NO:173 from nucleotide 317 to nucleotide 1366;
(d) a polynucleotide comprising the nucleotide sequence of SEQ ID NO:173 from nucleotide 227 to nucleotide 451;
(e) a polynucleotide comprising the nucleotide sequence of the full-length protein coding sequence of clone K289_{—}4 deposited with the ATCC under accession number 98117;
(f) a polynucleotide encoding the full-length protein encoded by the cDNA insert of clone K289_{—}4 deposited with the ATCC under accession number 98117;
(g) a polynucleotide comprising the nucleotide sequence of a mature protein coding sequence of clone K289_{—}4 deposited with the ATCC under accession number 98117;
(h) a polynucleotide encoding a mature protein encoded by the cDNA insert of clone K289_{—}4 deposited with the ATCC under accession number 98117;
(i) a polynucleotide encoding a protein comprising the amino acid sequence of SEQ ID NO:174;
(j) a polynucleotide encoding a protein comprising a fragment of the amino acid sequence of SEQ ID NO:174 having biological activity, the fragment comprising eight contiguous amino acids of SEQ ID NO:174;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:173.

Preferably, such polynucleotide comprises the nucleotide sequence of SEQ ID NO:173 from nucleotide 257 to nucleotide 1366; the nucleotide sequence of SEQ ID NO:173 from nucleotide 317 to nucleotide 1366; the nucleotide sequence of SEQ ID NO:173 from nucleotide 227 to nucleotide 451; the nucleotide sequence of the full-length protein coding sequence of clone K289_{—}4 deposited with the ATCC under accession number 98117; or the nucleotide sequence of a mature protein coding sequence of clone K289_{—}4 deposited with the ATCC under accession number 98117. In other preferred embodiments, the polynucleotide encodes the full-length or a mature protein encoded by the cDNA insert of clone K289_{—}4 deposited with the ATCC under accession number 98117. In yet other preferred embodiments, the present invention provides a polynucleotide encoding a protein comprising the amino acid sequence of SEQ ID NO:174 from amino acid 1 to amino acid 65. In further preferred embodiments, the present invention provides a polynucleotide encoding a protein comprising a fragment of the amino acid sequence of SEQ ID NO:174 having biological activity, the fragment preferably comprising eight (more preferably twenty, most preferably thirty) contiguous amino acids of SEQ ID NO:174, or a polynucleotide encoding a protein comprising a fragment of the amino acid sequence of SEQ ID NO:174 having biological activity, the fragment comprising the amino acid sequence from amino acid 180 to amino acid 189 of SEQ ID NO:174.

Other embodiments provide the gene corresponding to the cDNA sequence of SEQ ID NO:173.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:173, but excluding the poly(A) tail at the 3′ end of SEQ ID NO:173; and
      (ab) the nucleotide sequence of the cDNA insert of clone K289_{—}4 deposited with the ATCC under accession number 98117;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:173, but excluding the poly(A) tail at the 3′ end of SEQ ID NO:173; and
      (bb) the nucleotide sequence of the cDNA insert of clone K289_{—}4 deposited with the ATCC under accession number 98117;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:173, and extending contiguously from a nucleotide sequence corresponding to the 5′ end of SEQ ID NO:173 to a nucleotide sequence corresponding to the 3′ end of SEQ ID NO:173, but excluding the poly(A) tail at the 3′ end of SEQ ID NO:173. Also preferably the polynucleotide isolated according to the above process comprises a nucleotide sequence corresponding to the cDNA sequence of SEQ ID NO:173 from nucleotide 257 to nucleotide 1366, and extending contiguously from a nucleotide sequence corresponding to the 5′ end of said sequence of SEQ ID NO:173 from nucleotide 257 to nucleotide 1366, to a nucleotide sequence corresponding to the 3′ end of said sequence of SEQ ID NO:173 from nucleotide 257 to nucleotide 1366. Also preferably the polynucleotide isolated according to the above process comprises a nucleotide sequence corresponding to the cDNA sequence of SEQ ID NO:173 from nucleotide 317 to nucleotide 1366, and extending contiguously from a nucleotide sequence corresponding to the 5′ end of said sequence of SEQ ID NO:173 from nucleotide 317 to nucleotide 1366, to a nucleotide sequence corresponding to the 3′ end of said sequence of SEQ ID NO:173 from nucleotide 317 to nucleotide 1366. Also preferably the polynucleotide isolated according to the above process comprises a nucleotide sequence corresponding to the cDNA sequence of SEQ ID NO:173 from nucleotide 227 to nucleotide 451, and extending contiguously from a nucleotide sequence corresponding to the 5′ end of said sequence of SEQ ID NO:173 from nucleotide 227 to nucleotide 451, to a nucleotide sequence corresponding to the 3′ end of said sequence of SEQ ID NO:173 from nucleotide 227 to nucleotide 451.

In other embodiments, the present invention provides a composition comprising a protein, wherein said protein comprises an amino acid sequence selected from the group consisting of:
(a) the amino acid sequence of SEQ ID NO:174;
(b) the amino acid sequence of SEQ ID NO:174 from amino acid 1 to amino acid 65;
(c) a fragment of the amino acid sequence of SEQ ID NO:174, the fragment comprising eight contiguous amino acids of SEQ ID NO:174; and
(d) the amino acid sequence encoded by the cDNA insert of clone K289_{—}4 deposited with the ATCC under accession number 98117;
   the protein being substantially free from other mammalian proteins. Preferably such protein comprises the amino acid sequence of SEQ ID NO:174 or the amino acid sequence of SEQ ID NO:174 from amino acid I to amino acid 65. In further preferred embodiments, the present invention provides a protein comprising a fragment of the amino acid sequence of SEQ ID NO:174 having biological activity, the fragment preferably comprising eight (more preferably twenty, most preferably thirty) contiguous amino acids of SEQ ID NO:174, or a protein comprising a fragment of the amino acid sequence of SEQ ID NO:174 having biological activity, the fragment comprising the amino acid sequence from amino acid 180 to amino acid 189 of SEQ ID NO:174.

In one embodiment, the present invention provides a composition comprising an isolated polynucleotide selected from the group consisting of:
(a) a polynucleotide comprising the nucleotide sequence of SEQ ID NO:175;
(b) a polynucleotide comprising the nucleotide sequence of SEQ ID NO:175 from nucleotide 215 to nucleotide 946;
(c) a polynucleotide comprising the nucleotide sequence of SEQ ID NO:175 from nucleotide 578 to nucleotide 946;
(d) a polynucleotide comprising the nucleotide sequence of SEQ ID NO:175 from nucleotide 404 to nucleotide 706;
(e) a polynucleotide comprising the nucleotide sequence of the full-length protein coding sequence of clone K322_{—}4 deposited with the ATCC under accession number 98117;
(f) a polynucleotide encoding the full-length protein encoded by the cDNA insert of clone K322_{—}4 deposited with the ATCC under accession number 98117;
(g) a polynucleotide comprising the nucleotide sequence of a mature protein coding sequence of clone K322_{—}4 deposited with the ATCC under accession number 98117;
(h) a polynucleotide encoding a mature protein encoded by the cDNA insert of clone K322_{—}4 deposited with the ATCC under accession number 98117;
(i) a polynucleotide encoding a protein comprising the amino acid sequence of SEQ ID NO:176;
(j) a polynucleotide encoding a protein comprising a fragment of the amino acid sequence of SEQ ID NO:176 having biological activity, the fragment comprising eight contiguous amino acids of SEQ ID NO:176;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:175.

Preferably, such polynucleotide comprises the nucleotide sequence of SEQ ID NO:175 from nucleotide 215 to nucleotide 946; the nucleotide sequence of SEQ ID NO:175 from nucleotide 578 to nucleotide 946; the nucleotide sequence of SEQ ID NO:175 from nucleotide 404 to nucleotide 706; the nucleotide sequence of the full-length protein coding sequence of clone K322_{—}4 deposited with the ATCC under accession number 98117; or the nucleotide sequence of a mature protein coding sequence of clone K322_{—}4 deposited with the ATCC under accession number 98117. In other preferred embodiments, the polynucleotide encodes the full-length or a mature protein encoded by the cDNA insert of clone K322_{—}4 deposited with the ATCC under accession number 98117. In yet other preferred embodiments, the present invention provides a polynucleotide encoding a protein comprising the amino acid sequence of SEQ ID NO:176 from amino acid 95 to amino acid 164. In further preferred embodiments, the present invention provides a polynucleotide encoding a protein comprising a fragment of the amino acid sequence of SEQ ID NO:176 having biological activity, the fragment preferably comprising eight (more preferably twenty, most preferably thirty) contiguous amino acids of SEQ ID NO:176, or a polynucleotide encoding a protein comprising a fragment of the amino acid sequence of SEQ ID NO:176 having biological activity, the fragment comprising the amino acid sequence from amino acid 117 to amino acid 126 of SEQ ID NO:176.

Other embodiments provide the gene corresponding to the cDNA sequence of SEQ ID NO:175.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:175, but excluding the poly(A) tail at the 3′ end of SEQ ID NO:175; and
      (ab) the nucleotide sequence of the cDNA insert of clone K322_{—}4 deposited with the ATCC under accession number 98117;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:175, but excluding the poly(A) tail at the 3′ end of SEQ ID NO:175; and
      (bb) the nucleotide sequence of the cDNA insert of clone K322_{—}4 deposited with the ATCC under accession number 98117;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:175, and extending contiguously from a nucleotide sequence corresponding to the 5′ end of SEQ ID NO:175 to a nucleotide sequence corresponding to the 3′ end of SEQ ID NO:175, but excluding the poly(A) tail at the 3′ end of SEQ ID NO:175. Also preferably the polynucleotide isolated according to the above process comprises a nucleotide sequence corresponding to the cDNA sequence of SEQ ID NO:175 from nucleotide 215 to nucleotide 946, and extending contiguously from a nucleotide sequence corresponding to the 5 end of said sequence of SEQ [D NO:175 from nucleotide 215 to nucleotide 946, to a nucleotide sequence corresponding to the 3′ end of said sequence of SEQ ID NO:175 from nucleotide 215 to nucleotide 946. Also preferably the polynucleotide isolated according to the above process comprises a nucleotide sequence corresponding to the cDNA sequence of SEQ ID NO:175 from nucleotide 578 to nucleotide 946, and extending contiguously from a nucleotide sequence corresponding to the 5′ end of said sequence of SEQ ID NO:175 from nucleotide 578 to nucleotide 946, to a nucleotide sequence corresponding to the 3′ end of said sequence of SEQ ID NO:175 from nucleotide 578 to nucleotide 946. Also preferably the polynucleotide isolated according to the above process comprises a nucleotide sequence corresponding to the cDNA sequence of SEQ ID NO:175 from nucleotide 404 to nucleotide 706, and extending contiguously from a nucleotide sequence corresponding to the 5′ end of said sequence of SEQ ID NO:175 from nucleotide 404 to nucleotide 706, to a nucleotide sequence corresponding to the 3′ end of said sequence of SEQ ID NO:175 from nucleotide 404 to nucleotide 706.

In other embodiments, the present invention provides a composition comprising a protein, wherein said protein comprises an amino acid sequence selected from the group consisting of:
(a) the amino acid sequence of SEQ ID NO:176;
(b) the amino acid sequence of SEQ ID NO:176 from amino acid 95 to amino acid 164;
(c) a fragment of the amino acid sequence of SEQ ID NO:176, the fragment comprising eight contiguous amino acids of SEQ ID NO:176; and
(d) the amino acid sequence encoded by the cDNA insert of clone K322_{—}4 deposited with the ATCC under accession number 98117;
   the protein being substantially free from other mammalian proteins. Preferably such protein comprises the amino acid sequence of SEQ ID NO:176 or the amino acid sequence of SEQ ID NO:176 from amino acid 95 to amino acid 164. In further preferred embodiments, the present invention provides a protein comprising a fragment of the amino acid sequence of SEQ ID NO:176 having biological activity, the fragment preferably comprising eight (more preferably twenty, most preferably thirty) contiguous amino acids of SEQ ID NO:176, or a protein comprising a fragment of the amino acid sequence of SEQ ID NO:176 having biological activity, the fragment comprising the amino acid sequence from amino acid 117 to amino acid 126 of SEQ ID NO:176.

In one embodiment, the present invention provides a composition comprising an isolated polynucleotide selected from the group consisting of:
(a) a polynucleotide comprising the nucleotide sequence of SEQ ID NO:177;
(b) a polynucleotide comprising the nucleotide sequence of SEQ ID NO:177 from nucleotide 27 to nucleotide 1652;
(c) a polynucleotide comprising the nucleotide sequence of SEQ ID NO:177 from nucleotide 129 to nucleotide 1652;
(d) a polynucleotide comprising the nucleotide sequence of SEQ ID NO:177 from nucleotide 1 to nucleotide 413;
(e) a polynucleotide comprising the nucleotide sequence of the full-length protein coding sequence of clone AM349_{—}2 deposited with the ATCC under accession number 98155;
(f) a polynucleotide encoding the full-length protein encoded by the cDNA insert of clone AM349_{—}2 deposited with the ATCC under accession number 98155;
(g) a polynucleotide comprising the nucleotide sequence of a mature protein coding sequence of clone AM349_{—}2 deposited with the ATCC under accession number 98155;
(h) a polynucleotide encoding a mature protein encoded by the cDNA insert of clone AM349_{—}2 deposited with the ATCC under accession number 98155;
(i) a polynucleotide encoding a protein comprising the amino acid sequence of SEQ ID NO:178;
(j) a polynucleotide encoding a protein comprising a fragment of the amino acid sequence of SEQ ID NO:178 having biological activity, the fragment comprising eight contiguous amino acids of SEQ ID NO:178;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:177.

Preferably, such polynucleotide comprises the nucleotide sequence of SEQ ID NO:177 from nucleotide 27 to nucleotide 1652; the nucleotide sequence of SEQ ID NO:177 from nucleotide 129 to nucleotide 1652; the nucleotide sequence of SEQ ID NO:177 from nucleotide I to nucleotide 413; the nucleotide sequence of the full-length protein coding sequence of clone AM349_{—}2 deposited with the ATCC under accession number 98155; or the nucleotide sequence of a mature protein coding sequence of clone AM349_{—}2 deposited with the ATCC under accession number 98155. In other preferred embodiments, the polynucleotide encodes the full-length or a mature protein encoded by the cDNA insert of clone AM349_{—}2 deposited with the ATCC under accession number 98155. In yet other preferred embodiments, the present invention provides a polynucleotide encoding a protein comprising the amino acid sequence of SEQ ID NO:178 from amino acid 1 to amino acid 129. In further preferred embodiments, the present invention provides a polynucleotide encoding a protein comprising a fragment of the amino acid sequence of SEQ ID NO:178 having biological activity, the fragment preferably comprising eight (more preferably twenty, most preferably thirty) contiguous amino acids of SEQ ID NO:178, or a polynucleotide encoding a protein comprising a fragment of the amino acid sequence of SEQ ID NO:178 having biological activity, the fragment comprising the amino acid sequence from amino acid 266 to amino acid 275 of SEQ ID NO:178.

Other embodiments provide the gene corresponding to the cDNA sequence of SEQ ID NO:177.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:177, but excluding the poly(A) tail at the 3′ end of SEQ ID NO:177; and
      (ab) the nucleotide sequence of the cDNA insert of clone AM349_{—}2 deposited with the ATCC under accession number 98155;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:177, but excluding the poly(A) tail at the 3′ end of SEQ ID NO:177; and
      (bb) the nucleotide sequence of the cDNA insert of clone AM349_{—}2 deposited with the ATCC under accession number 98155;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:177, and extending contiguously from a nucleotide sequence corresponding to the 5′ end of SEQ ID NO:177 to a nucleotide sequence corresponding to the 3′ end of SEQ ID NO:177, but excluding the poly(A) tail at the 3′ end of SEQ ID NO:177. Also preferably the polynucleotide isolated according to the above process comprises a nucleotide sequence corresponding to the cDNA sequence of SEQ ID NO:177 from nucleotide 27 to nucleotide 1652, and extending contiguously from a nucleotide sequence corresponding to the 5′ end of said sequence of SEQ ID NO:177 from nucleotide 27 to nucleotide 1652, to a nucleotide sequence corresponding to the 3′ end of said sequence of SEQ ID NO:177 from nucleotide 27 to nucleotide 1652. Also preferably the polynucleotide isolated according to the above process comprises a nucleotide sequence corresponding to the cDNA sequence of SEQ ID NO:177 from nucleotide 129 to nucleotide 1652, and extending contiguously from a nucleotide sequence corresponding to the 5′ end of said sequence of SEQ ID NO:177 from nucleotide 129 to nucleotide 1652, to a nucleotide sequence corresponding to the 3′ end of said sequence of SEQ ID NO:177 from nucleotide 129 to nucleotide 1652. Also preferably the polynucleotide isolated according to the above process comprises a nucleotide sequence corresponding to the cDNA sequence of SEQ ID NO:177 from nucleotide 1 to nucleotide 413, and extending contiguously from a nucleotide sequence corresponding to the 5′ end of said sequence of SEQ ID NO:177 from nucleotide 1 to nucleotide 413, to a nucleotide sequence corresponding to the 3′ end of said sequence of SEQ ID NO:177 from nucleotide 1 to nucleotide 413.

In other embodiments, the present invention provides a composition comprising a protein, wherein said protein comprises an amino acid sequence selected from the group consisting of:
(a) the amino acid sequence of SEQ ID NO:178;
(b) the amino acid sequence of SEQ ID NO:178 from amino acid 1 to amino acid 129;
(c) a fragment of the amino acid sequence of SEQ ID NO:178, the fragment comprising eight contiguous amino acids of SEQ ID NO:178; and
(d) the amino acid sequence encoded by the cDNA insert of clone AM349_{—}2 deposited with the ATCC under accession number 98155;
   the protein being substantially free from other mammalian proteins. Preferably such protein comprises the amino acid sequence of SEQ ID NO:178 or the amino acid sequence of SEQ ID NO:178 from amino acid 1 to amino acid 129. In further preferred embodiments, the present invention provides a protein comprising a fragment of the amino acid sequence of SEQ ID NO:178 having biological activity, the fragment preferably comprising eight (more preferably twenty, most preferably thirty) contiguous amino acids of SEQ ID NO:178, or a protein comprising a fragment of the amino acid sequence of SEQ ID NO:178 having biological activity, the fragment comprising the amino acid sequence from amino acid 266 to amino acid 275 of SEQ ID NO:178.

In one embodiment, the present invention provides a composition comprising an isolated polynucleotide selected from the group consisting of:
(a) a polynucleotide comprising the nucleotide sequence of SEQ ID NO:179;
(b) a polynucleotide comprising the nucleotide sequence of SEQ ID NO:179 from nucleotide 788 to nucleotide 1153;
(c) a polynucleotide comprising the nucleotide sequence of SEQ ID NO:179 from nucleotide 1881 to nucleotide 2146;
(d) a polynucleotide comprising the nucleotide sequence of the full-length protein coding sequence of clone AR310_{—}3 deposited with the ATCC under accession number 98155;
(e) a polynucleotide encoding the full-length protein encoded by the cDNA insert of clone AR310_{—}3 deposited with the ATCC under accession number 98155;
(f) a polynucleotide comprising the nucleotide sequence of a mature protein coding sequence of clone AR310_{—}3 deposited with the ATCC under accession number 98155;
(g) a polynucleotide encoding a mature protein encoded by the cDNA insert of clone AR310_{—}3 deposited with the ATCC under accession number 98155;
(h) a polynucleotide encoding a protein comprising the amino acid sequence of SEQ ID NO:180;
(i) a polynucleotide encoding a protein comprising a fragment of the amino acid sequence of SEQ ID NO:180 having biological activity, the fragment comprising eight contiguous amino acids of SEQ ID NO:180;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:179.

Preferably, such polynucleotide comprises the nucleotide sequence of SEQ ID NO:179 from nucleotide 788 to nucleotide 1153; the nucleotide sequence of SEQ ID NO:179 from nucleotide 1881 to nucleotide 2146; the nucleotide sequence of the full-length protein coding sequence of clone AR310_{—}3 deposited with the ATCC under accession number 98155; or the nucleotide sequence of a mature protein coding sequence of clone AR310_{—}3 deposited with the ATCC under accession number 98155. In other preferred embodiments, the polynucleotide encodes the full-length or a mature protein encoded by the cDNA insert of clone AR310_{—}3 deposited with the ATCC under accession number 98155. In further preferred embodiments, the present invention provides a polynucleotide encoding a protein comprising a fragment of the amino acid sequence of SEQ ID NO:180 having biological activity, the fragment preferably comprising eight (more preferably twenty, most preferably thirty) contiguous amino acids of SEQ ID NO:180, or a polynucleotide encoding a protein comprising a fragment of the amino acid sequence of SEQ ID NO:180 having biological activity, the fragment comprising the amino acid sequence from amino acid 56 to amino acid 65 of SEQ ID NO:180.

Other embodiments provide the gene corresponding to the cDNA sequence of SEQ ID NO:179.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:179, but excluding the poly(A) tail at the 3′ end of SEQ ID NO:179; and
      (ab) the nucleotide sequence of the cDNA insert of clone AR310_{—}3 deposited with the ATCC under accession number 98155;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:179, but excluding the poly(A) tail at the 3′ end of SEQ ID NO:179; and
      (bb) the nucleotide sequence of the cDNA insert of clone AR310_{—}3 deposited with the ATCC under accession number 98155;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:179, and extending contiguously from a nucleotide sequence corresponding to the 5′ end of SEQ ID NO:179 to a nucleotide sequence corresponding to the 3′ end of SEQ ID NO:179, but excluding the poly(A) tail at the 3′ end of SEQ ID NO:179. Also preferably the polynucleotide isolated according to the above process comprises a nucleotide sequence corresponding to the cDNA sequence of SEQ ID NO:179 from nucleotide 788 to nucleotide 1153, and extending contiguously from a nucleotide sequence corresponding to the 5′ end of said sequence of SEQ ID NO:179 from nucleotide 788 to nucleotide 1153, to a nucleotide sequence corresponding to the 3′ end of said sequence of SEQ ID NO:179 from nucleotide 788 to nucleotide 1153. Also preferably the polynucleotide isolated according to the above process comprises a nucleotide sequence corresponding to the cDNA sequence of SEQ ID NO:179 from nucleotide 1881 to nucleotide 2146, and extending contiguously from a nucleotide sequence corresponding to the 5′ end of said sequence of SEQ ID NO:179 from nucleotide 1881 to nucleotide 2146, to a nucleotide sequence corresponding to the 3′ end of said sequence of SEQ ID NO:179 from nucleotide 1881 to nucleotide 2146.

In other embodiments, the present invention provides a composition comprising a protein, wherein said protein comprises an amino acid sequence selected from the group consisting of:
(a) the amino acid sequence of SEQ ID NO:180;
(b) a fragment of the amino acid sequence of SEQ ID NO:180, the fragment comprising eight contiguous amino acids of SEQ ID NO:180; and
(c) the amino acid sequence encoded by the cDNA insert of clone AR310_{—}3 deposited with the ATCC under accession number 98155;
   the protein being substantially free from other mammalian proteins. Preferably such protein comprises the amino acid sequence of SEQ ID NO:180. In further preferred embodiments, the present invention provides a protein comprising a fragment of the amino acid sequence of SEQ ID NO:180 having biological activity, the fragment preferably comprising eight (more preferably twenty, most preferably thirty) contiguous amino acids of SEQ ID NO:180, or a protein comprising a fragment of the amino acid sequence of SEQ ID NO:180 having biological activity, the fragment comprising the amino acid sequence from amino acid 56 to amino acid 65 of SEQ ID NO:180.

In one embodiment, the present invention provides a composition comprising an isolated polynucleotide selected from the group consisting of:
(a) a polynucleotide comprising the nucleotide sequence of SEQ ID NO:181;
(b) a polynucleotide comprising the nucleotide sequence of SEQ ID NO:181 from nucleotide 432 to nucleotide 647;
(c) a polynucleotide comprising the nucleotide sequence of SEQ ID NO:181 from nucleotide 531 to nucleotide 647;
(d) a polynucleotide comprising the nucleotide sequence of SEQ ID NO:181 from nucleotide 305 to nucleotide 587;
(e) a polynucleotide comprising the nucleotide sequence of the full-length protein coding sequence of clone AS186_{—}3 deposited with the ATCC under accession number 98155;
(f) a polynucleotide encoding the full-length protein encoded by the cDNA insert of clone AS186_{—}3 deposited with the ATCC under accession number 98155;
(g) a polynucleotide comprising the nucleotide sequence of a mature protein coding sequence of clone AS186_{—}3 deposited with the ATCC under accession number 98155;
(h) a polynucleotide encoding a mature protein encoded by the cDNA insert of clone AS186_{—}3 deposited with the ATCC under accession number 98155;
(i) a polynucleotide encoding a protein comprising the amino acid sequence of SEQ ID NO:182;
(j) a polynucleotide encoding a protein comprising a fragment of the amino acid sequence of SEQ ID NO:182 having biological activity, the fragment comprising eight contiguous amino acids of SEQ ID NO:182;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:181.

Preferably, such polynucleotide comprises the nucleotide sequence of SEQ ID NO:181 from nucleotide 432 to nucleotide 647; the nucleotide sequence of SEQ ID NO:181 from nucleotide 531 to nucleotide 647; the nucleotide sequence of SEQ ID NO:181 from nucleotide 305 to nucleotide 587; the nucleotide sequence of the full-length protein coding sequence of clone AS186_{—}3 deposited with the ATCC under accession number 98155; or the nucleotide sequence of a mature protein coding sequence of clone AS186_{—}3 deposited with the ATCC under accession number 98155. In other preferred embodiments, the polynucleotide encodes the full-length or a mature protein encoded by the cDNA insert of clone AS186_{—}3 deposited with the ATCC under accession number 98155. In yet other preferred embodiments, the present invention provides a polynucleotide encoding a protein comprising the amino acid sequence of SEQ ID NO:182 from amino acid 1 to amino acid 52. In further preferred embodiments, the present invention provides a polynucleotide encoding a protein comprising a fragment of the amino acid sequence of SEQ ID NO:182 having biological activity, the fragment preferably comprising eight (more preferably twenty, most preferably thirty) contiguous amino acids of SEQ ID NO:182, or a polynucleotide encoding a protein comprising a fragment of the amino acid sequence of SEQ ID NO:182 having biological activity, the fragment comprising the amino acid sequence from amino acid 31 to amino acid 40 of SEQ ID NO:182.

Other embodiments provide the gene corresponding to the cDNA sequence of SEQ ID NO:181.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:181, but excluding the poly(A) tail at the 3′ end of SEQ ID NO:181; and
      (ab) the nucleotide sequence of the cDNA insert of clone AS186_{—}3 deposited with the ATCC under accession number 98155;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:181, but excluding the poly(A) tail at the 3′ end of SEQ ID NO:181; and
      (bb) the nucleotide sequence of the cDNA insert of clone AS186_{—}3 deposited with the ATCC under accession number 98155;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:181, and extending contiguously from a nucleotide sequence corresponding to the 5′ end of SEQ ID NO:181 to a nucleotide sequence corresponding to the 3′ end of SEQ ID NO:181, but excluding the poly(A) tail at the 3′ end of SEQ ID NO:181. Also preferably the polynucleotide isolated according to the above process comprises a nucleotide sequence corresponding to the cDNA sequence of SEQ ID NO:181 from nucleotide 432 to nucleotide-647, and extending contiguously from a nucleotide sequence corresponding to the 5′ end of said sequence of SEQ ID NO:181 from nucleotide 432 to nucleotide 647, to a nucleotide sequence corresponding to the 3′ end of said sequence of SEQ ID NO:181 from nucleotide 432 to nucleotide 647. Also preferably the polynucleotide isolated according to the above process comprises a nucleotide sequence corresponding to the cDNA sequence of SEQ ID NO:181 from nucleotide 531 to nucleotide 647, and extending contiguously from a nucleotide sequence corresponding to the 5′ end of said sequence of SEQ ID NO:181 from nucleotide 531 to nucleotide 647, to a nucleotide sequence corresponding to the 3′ end of said sequence of SEQ ID NO:181 from nucleotide 531 to nucleotide 647. Also preferably the polynucleotide isolated according to the above process comprises a nucleotide sequence corresponding to the cDNA sequence of SEQ ID NO:181 from nucleotide 305 to nucleotide 587, and extending contiguously from a nucleotide sequence corresponding to the 5′ end of said sequence of SEQ ID NO:181 from nucleotide 305 to nucleotide 587, to a nucleotide sequence corresponding to the 3′ end of said sequence of SEQ ID NO:181 from nucleotide 305 to nucleotide 587.

In other embodiments, the present invention provides a composition comprising a protein, wherein said protein comprises an amino acid sequence selected from the group consisting of:
(a) the amino acid sequence of SEQ ID NO:182;
(b) the amino acid sequence of SEQ ID NO:182 from amino acid 1 to amino acid 52;
(c) a fragment of the amino acid sequence of SEQ ID NO:182, the fragment comprising eight contiguous amino acids of SEQ ID NO:182; and
(d) the amino acid sequence encoded by the cDNA insert of clone AS186_{—}3 deposited with the ATCC under accession number 98155;
   the protein being substantially free from other mammalian proteins. Preferably such protein comprises the amino acid sequence of SEQ ID NO:182 or the amino acid sequence of SEQ ID NO:182 from amino acid 1 to amino acid 52. In further preferred embodiments, the present invention provides a protein comprising a fragment of the amino acid sequence of SEQ ID NO:182 having biological activity, the fragment preferably comprising eight (more preferably twenty, most preferably thirty) contiguous amino acids of SEQ ID NO:182, or a protein comprising a fragment of the amino acid sequence of SEQ ID NO:182 having biological activity, the fragment comprising the amino acid sequence from amino acid 31 to amino acid 40 of SEQ ID NO:182.

In one embodiment, the present invention provides a composition comprising an isolated polynucleotide selected from the group consisting of:
(a) a polynucleotide comprising the nucleotide sequence of SEQ ID NO:183;
(b) a polynucleotide comprising the nucleotide sequence of SEQ ID NO:183 from nucleotide 140 to nucleotide 1498;
(c) a polynucleotide comprising the nucleotide sequence of SEQ ID NO:183 from nucleotide 185 to nucleotide 1498;
(d) a polynucleotide comprising the nucleotide sequence of SEQ ID NO:183 from nucleotide 132 to nucleotide 457;
(e) a polynucleotide comprising the nucleotide sequence of the full-length protein coding sequence of clone AY160_{—}2 deposited with the ATCC under accession number 98155;
(f) a polynucleotide encoding the full-length protein encoded by the cDNA insert of clone AY160_{—}2 deposited with the ATCC under accession number 98155;
(g) a polynucleotide comprising the nucleotide sequence of a mature protein coding sequence of clone AY160_{—}2 deposited with the ATCC under accession number 98155;
(h) a polynucleotide encoding a mature protein encoded by the cDNA insert of clone AY160_{—}2 deposited with the ATCC under accession number 98155;
(i) a polynucleotide encoding a protein comprising the amino acid sequence of SEQ ID NO:184;
(j) a polynucleotide encoding a protein comprising a fragment of the amino acid sequence of SEQ ID NO:184 having biological activity, the fragment comprising eight contiguous amino acids of SEQ ID NO:184;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:183.

Preferably, such polynucleotide comprises the nucleotide sequence of SEQ ID NO:183 from nucleotide 140 to nucleotide 1498; the nucleotide sequence of SEQ ID NO:183 from nucleotide 185 to nucleotide 1498; the nucleotide sequence of SEQ ID NO:183 from nucleotide 132 to nucleotide 457; the nucleotide sequence of the full-length protein coding sequence of clone AY160_{—}2 deposited with the ATCC under accession number 98155; or the nucleotide sequence of a mature protein coding sequence of clone AY160_{—}2 deposited with the ATCC under accession number 98155. In other preferred embodiments, the polynucleotide encodes the full-length or a mature protein encoded by the cDNA insert of clone AY160_{—}2 deposited with the ATCC under accession number 98155. In yet other preferred embodiments, the present invention provides a polynucleotide encoding a protein comprising the amino acid sequence of SEQ ID NO:184 from amino acid 1 to amino acid 106. In further preferred embodiments, the present invention provides a polynucleotide encoding a protein comprising a fragment of the amino acid sequence of SEQ ID NO:184 having biological activity, the fragment preferably comprising eight (more preferably twenty, most preferably thirty) contiguous amino acids of SEQ ID NO:184, or a polynucleotide encoding a protein comprising a fragment of the amino acid sequence of SEQ ID NO:184 having biological activity, the fragment comprising the amino acid sequence from amino acid 221 to amino acid 230 of SEQ ID NO:184.

Other embodiments provide the gene corresponding to the cDNA sequence of SEQ ID NO:183.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:183, but excluding the poly(A) tail at the 3′ end of SEQ ID NO:183; and
      (ab) the nucleotide sequence of the cDNA insert of clone AY160_{—}2 deposited with the ATCC under accession number 98155;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:183, but excluding the poly(A) tail at the 3′ end of SEQ ID NO:183; and
      (bb) the nucleotide sequence of the cDNA insert of clone AY160_{—}2 deposited with the ATCC under accession number 98155;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:183, and extending contiguously from a nucleotide sequence corresponding to the 5′ end of SEQ ID NO:183 to a nucleotide sequence corresponding to the 3′ end of SEQ ID NO:183, but excluding the poly(A) tail at the 3′ end of SEQ ID NO:183. Also preferably the polynucleotide isolated according to the above process comprises a nucleotide sequence corresponding to the cDNA sequence of SEQ ID NO:183 from nucleotide 140 to nucleotide 1498, and extending contiguously from a nucleotide sequence corresponding to the 5′ end of said sequence of SEQ ID NO:183 from nucleotide 140 to nucleotide 1498, to a nucleotide sequence corresponding to the 3′ end of said sequence of SEQ ID NO:183 from nucleotide 140 to nucleotide 1498. Also preferably the polynucleotide isolated according to the above process comprises a nucleotide sequence corresponding to the cDNA sequence of SEQ ID NO:183 from nucleotide 185 to nucleotide 1498, and extending contiguously from a nucleotide sequence corresponding to the 5′ end of said sequence of SEQ ID NO:183 from nucleotide 185 to nucleotide 1498, to a nucleotide sequence corresponding to the 3′ end of said sequence of SEQ ID NO:183 from nucleotide 185 to nucleotide 1498. Also preferably the polynucleotide isolated according to the above process comprises a nucleotide sequence corresponding to the cDNA sequence of SEQ ID NO:183 from nucleotide 132 to nucleotide 457, and extending contiguously from a nucleotide sequence corresponding to the 5′ end of said sequence of SEQ ID NO:183 from nucleotide 132 to nucleotide 457, to a nucleotide sequence corresponding to the 3′ end of said sequence of SEQ ID NO:183 from nucleotide 132 to nucleotide 457.

In other embodiments, the present invention provides a composition comprising a protein, wherein said protein comprises an amino acid sequence selected from the group consisting of:
(a) the amino acid sequence of SEQ ID NO:184;
(b) the amino acid sequence of SEQ ID NO:184 from amino acid 1 to amino acid 106;
(c) a fragment of the amino acid sequence of SEQ ID NO:184, the fragment comprising eight contiguous amino acids of SEQ ID NO:184; and
(d) the amino acid sequence encoded by the cDNA insert of clone AY160_{—}2 deposited with the ATCC under accession number 98155;
   the protein being substantially free from other mammalian proteins. Preferably such protein comprises the amino acid sequence of SEQ ID NO:184 or the amino acid sequence of SEQ ID NO:184 from amino acid 1 to amino acid 106. In further preferred embodiments, the present invention provides a protein comprising a fragment of the amino acid sequence of SEQ ID NO:184 having biological activity, the fragment preferably comprising eight (more preferably twenty, most preferably thirty) contiguous amino acids of SEQ ID NO:184, or a protein comprising a fragment of the amino acid sequence of SEQ ID NO:184 having biological activity, the fragment comprising the amino acid sequence from amino acid 221 to amino acid 230 of SEQ ID NO:184.

In one embodiment, the present invention provides a composition comprising an isolated polynucleotide selected from the group consisting of:
(a) a polynucleotide comprising the nucleotide sequence of SEQ ID NO:185;
(b) a polynucleotide comprising the nucleotide sequence of SEQ ID NO:185 from nucleotide 85 to nucleotide 582;
(c) a polynucleotide comprising the nucleotide sequence of SEQ ID NO:185 from nucleotide 96 to nucleotide 454;
(d) a polynucleotide comprising the nucleotide sequence of the full-length protein coding sequence of clone BD127_{—}16 deposited with the ATCC under accession number 98155;
(e) a polynucleotide encoding the full-length protein encoded by the cDNA insert of clone BD127_{—}16 deposited with the ATCC under accession number 98155;
(f) a polynucleotide comprising the nucleotide sequence of a mature protein coding sequence of clone BD127_{—}16 deposited with the ATCC under accession number 98155;
(g) a polynucleotide encoding a mature protein encoded by the cDNA insert of clone BD127_{—}16 deposited with the ATCC under accession number 98155;
(h) a polynucleotide encoding a protein comprising the amino acid sequence of SEQ ID NO:186;
(i) a polynucleotide encoding a protein comprising a fragment of the amino acid sequence of SEQ ID NO:186 having biological activity, the fragment comprising eight contiguous amino acids of SEQ ID NO:186;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:185.

Preferably, such polynucleotide comprises the nucleotide sequence of SEQ ID NO:185 from nucleotide 85 to nucleotide 582; the nucleotide sequence of SEQ ID NO:185 from nucleotide 96 to nucleotide 454; the nucleotide sequence of the full-length protein coding sequence of clone BD127_{—}16 deposited with the ATCC under accession number 98155; or the nucleotide sequence of a mature protein coding sequence of clone BD127_{—}16 deposited with the ATCC under accession number 98155. In other preferred embodiments, the polynucleotide encodes the full-length or a mature protein encoded by the cDNA insert of clone BD127_{—}16 deposited with the ATCC under accession number 98155. In yet other preferred embodiments, the present invention provides a polynucleotide encoding a protein comprising the amino acid sequence of SEQ ID NO:186 from amino acid 5 to amino acid 123. In further preferred embodiments, the present invention provides a polynucleotide encoding a protein comprising a fragment of the amino acid sequence of SEQ ID NO:186 having biological activity, the fragment preferably comprising eight (more preferably twenty, most preferably thirty) contiguous amino acids of SEQ ID NO:186, or a polynucleotide encoding a protein comprising a fragment of the amino acid sequence of SEQ ID NO:186 having biological activity, the fragment comprising the amino acid sequence from amino acid 78 to amino acid 87 of SEQ ID NO:186.

Other embodiments provide the gene corresponding to the cDNA sequence of SEQ ID NO:185.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:185, but excluding the poly(A) tail at the 3′ end of SEQ ID NO:185; and
      (ab) the nucleotide sequence of the cDNA insert of clone BD127_{—}16 deposited with the ATCC under accession number 98155;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:185, but excluding the poly(A) tail at the 3′ end of SEQ ID NO:185; and
      (bb) the nucleotide sequence of the cDNA insert of clone BD127_{—}16 deposited with the ATCC under accession number 98155;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:185, and extending contiguously from a nucleotide sequence corresponding to the 5′ end of SEQ ID NO:185 to a nucleotide sequence corresponding to the 3′ end of SEQ ID NO:185, but excluding the poly(A) tail at the 3′ end of SEQ ID NO:185. Also preferably the polynucleotide isolated according to the above process comprises a nucleotide sequence corresponding to the cDNA sequence of SEQ ID NO:185 from nucleotide 85 to nucleotide 582, and extending contiguously from a nucleotide sequence corresponding to the 5′ end of said sequence of SEQ ID NO:185 from nucleotide 85 to nucleotide 582, to a nucleotide sequence corresponding to the 3′ end of said sequence of SEQ ID NO:185 from nucleotide 85 to nucleotide 582. Also preferably the polynucleotide isolated according to the above process comprises a nucleotide sequence corresponding to the cDNA sequence of SEQ ID NO:185 from nucleotide 96 to nucleotide 454, and extending contiguously from a nucleotide sequence corresponding to the 5′ end of said sequence of SEQ ID NO:185 from nucleotide 96 to nucleotide 454, to a nucleotide sequence corresponding to the 3′ end of said sequence of SEQ ID NO:185 from nucleotide 96 to nucleotide 454.

In other embodiments, the present invention provides a composition comprising a protein, wherein said protein comprises an amino acid sequence selected from the group consisting of:
(a) the amino acid sequence of SEQ ID NO:186;
(b) the amino acid sequence of SEQ ID NO:186 from amino acid 5 to amino acid 123;
(c) a fragment of the amino acid sequence of SEQ ID NO:186, the fragment comprising eight contiguous amino acids of SEQ ID NO:186; and
(d) the amino acid sequence encoded by the cDNA insert of clone BD127_{—}16 deposited with the ATCC under accession number 98155;
   the protein being substantially free from other mammalian proteins. Preferably such protein comprises the amino acid sequence of SEQ ID NO:186 or the amino acid sequence of SEQ ID NO:186 from amino acid 5 to amino acid 123. In further preferred embodiments, the present invention provides a protein comprising a fragment of the amino acid sequence of SEQ ID NO:186 having biological activity, the fragment preferably comprising eight (more preferably twenty, most preferably thirty) contiguous amino acids of SEQ ID NO:186, or a protein comprising a fragment of the amino acid sequence of SEQ ID NO:186 having biological activity, the fragment comprising the amino acid sequence from amino acid 78 to amino acid 87 of SEQ ID NO:186.

In one embodiment, the present invention provides a composition comprising an isolated polynucleotide selected from the group consisting of:
(a) a polynucleotide comprising the nucleotide sequence of SEQ ID NO:187;
(b) a polynucleotide comprising the nucleotide sequence of SEQ ID NO:187 from nucleotide 236 to nucleotide 1480;
(c) a polynucleotide comprising the nucleotide sequence of SEQ ID NO:187 from nucleotide 564 to nucleotide 800;
(d) a polynucleotide comprising the nucleotide sequence of the full-length protein coding sequence of clone BL205_{—}14 deposited with the ATCC under accession number 98155;
(e) a polynucleotide encoding the full-length protein encoded by the cDNA insert of clone BL205_{—}14 deposited with the ATCC under accession number 98155;
(f) a polynucleotide comprising the nucleotide sequence of a mature protein coding sequence of clone BL205_{—}14 deposited with the ATCC under accession number 98155;
(g) a polynucleotide encoding a mature protein encoded by the cDNA insert of clone BL205_{—}14 deposited with the ATCC under accession number 98155;
(h) a polynucleotide encoding a protein comprising the amino acid sequence of SEQ ID NO:188;
(i) a polynucleotide encoding a protein comprising a fragment of the amino acid sequence of SEQ ID NO:188 having biological activity, the fragment comprising eight contiguous amino acids of SEQ ID NO:188;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:187.

Preferably, such polynucleotide comprises the nucleotide sequence of SEQ ID NO:187 from nucleotide 236 to nucleotide 1480; the nucleotide sequence of SEQ ID NO:187 from nucleotide 564 to nucleotide 800; the nucleotide sequence of the full-length protein coding sequence of clone BL205_{—}14 deposited with the ATCC under accession number 98155; or the nucleotide sequence of a mature protein coding sequence of clone BL205_{—}14 deposited with the ATCC under accession number 98155. In other preferred embodiments, the polynucleotide encodes the full-length or a mature protein encoded by the cDNA insert of clone BL205_{—}14 deposited with the ATCC under accession number 98155. In yet other preferred embodiments, the present invention provides a polynucleotide encoding a protein comprising the ammo acid sequence of SEQ ID NO:188 from amino acid 89 to amino acid 188. In further preferred embodiments, the present invention provides a polynucleotide encoding a protein comprising a fragment of the amino acid sequence of SEQ ID NO:188 having biological activity, the fragment preferably comprising eight (more preferably twenty, most preferably thirty) contiguous amino acids of SEQ ID NO:188, or a polynucleotide encoding a protein comprising a fragment of the amino acid sequence of SEQ ID NO:188 having biological activity, the fragment comprising the amino acid sequence from amino acid 202 to amino acid 211 of SEQ ID NO:188.

Other embodiments provide the gene corresponding to the cDNA sequence of SEQ ID NO:187.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:187, but excluding the poly(A) tail at the 3′ end of SEQ ID NO:187; and
      (ab) the nucleotide sequence of the cDNA insert of clone BL205_{—}14 deposited with the ATCC under accession number 98155;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:187, but excluding the poly(A) tail at the 3′ end of SEQ ID NO:187; and
      (bb) the nucleotide sequence of the cDNA insert of clone BL205_{—}14 deposited with the ATCC under accession number 98155;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:187, and extending contiguously from a nucleotide sequence corresponding to the 5′ end of SEQ ID NO:187 to a nucleotide sequence corresponding to the 3′ end of SEQ ID NO:187, but excluding the poly(A) tail at the 3′ end of SEQ ID NO:187. Also preferably the polynucleotide isolated according to the above process comprises a nucleotide sequence corresponding to the cDNA sequence of SEQ ID NO:187 from nucleotide 236 to nucleotide 1480, and extending contiguously from a nucleotide sequence corresponding to the 5′ end of said sequence of SEQ ID NO:187 from nucleotide 236 to nucleotide 1480, to a nucleotide sequence corresponding to the 3′ end of said sequence of SEQ ID NO:187 from nucleotide 236 to nucleotide 1480. Also preferably the polynucleotide isolated according to the above process comprises a nucleotide sequence corresponding to the cDNA sequence of SEQ ID NO:187 from nucleotide 564 to nucleotide 800, and extending contiguously from a nucleotide sequence corresponding to the 5′ end of said sequence of SEQ ID NO:187 from nucleotide 564 to nucleotide 800, to a nucleotide sequence corresponding to the 3′ end of said sequence of SEQ ID NO:187 from nucleotide 564 to nucleotide 800.

In other embodiments, the present invention provides a composition comprising a protein, wherein said protein comprises an amino acid sequence selected from the group consisting of:
(a) the amino acid sequence of SEQ ID NO:188;
(b) the amino acid sequence of SEQ ID NO:188 from amino acid 89 to amino acid 188;
(c) a fragment of the amino acid sequence of SEQ ID NO:188, the fragment comprising eight contiguous amino acids of SEQ ID NO:188; and
(d) the amino acid sequence encoded by the cDNA insert of clone BL205_{—}14 deposited with the ATCC under accession number 98155;
   the protein being substantially free from other mammalian proteins. Preferably such protein comprises the amino acid sequence of SEQ ID NO:188 or the amino acid sequence of SEQ ID NO:188 from amino acid 89 to amino acid 188. In further preferred embodiments, the present invention provides a protein comprising a fragment of the amino acid sequence of SEQ ID NO:188 having biological activity, the fragment preferably comprising eight (more preferably twenty, most preferably thirty) contiguous amino acids of SEQ ID NO:188, or a protein comprising a fragment of the amino acid sequence of SEQ ID NO:188 having biological activity, the fragment comprising the amino acid sequence from amino acid 202 to amino acid 211 of SEQ ID NO:188.

In one embodiment, the present invention provides a composition comprising an isolated polynucleotide selected from the group consisting of:
(a) a polynucleotide comprising the nucleotide sequence of SEQ ID NO:189;
(b) a polynucleotide comprising the nucleotide sequence of SEQ ID NO:189 from nucleotide 69 to nucleotide 371;
(c) a polynucleotide comprising the nucleotide sequence of SEQ ID NO:189 from nucleotide 109 to nucleotide 350;
(d) a polynucleotide comprising the nucleotide sequence of the full-length protein coding sequence of clone H438_{—}1 deposited with the ATCC under accession number 98140;
(e) a polynucleotide encoding the full-length protein encoded by the cDNA insert of clone H438_{—}1 deposited with the ATCC under accession number 98140;
(f) a polynucleotide comprising the nucleotide sequence of a mature protein coding sequence of clone H438_{—}1 deposited with the ATCC under accession number 98140;
(g) a polynucleotide encoding a mature protein encoded by the cDNA insert of clone H438_{—}1 deposited with the ATCC under accession number 98140;
(h) a polynucleotide encoding a protein comprising the amino acid sequence of SEQ ID NO:190;
(i) a polynucleotide encoding a protein comprising a fragment of the amino acid sequence of SEQ ID NO:190 having biological activity, the fragment comprising eight contiguous amino acids of SEQ ID NO:190;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-ij) and that has a length that is at least 25% of the length of SEQ ID NO:189.

Preferably, such polynucleotide comprises the nucleotide sequence of SEQ ID NO:189 from nucleotide 69 to nucleotide 371; the nucleotide sequence of SEQ ID NO:189 from nucleotide 109 to nucleotide 350; the nucleotide sequence of the full-length protein coding sequence of clone H438_{—}1 deposited with the ATCC under accession number 98140; or the nucleotide sequence of a mature protein coding sequence of clone H438_{—}1 deposited with the ATCC under accession number 98140. In other preferred embodiments, the polynucleotide encodes the full-length or a mature protein encoded by the cDNA insert of clone H438_{—}1 deposited with the ATCC under accession number 98140. In yet other preferred embodiments, the present invention provides a polynucleotide encoding a protein comprising the amino acid sequence of SEQ ID NO:190 from amino acid 17 to amino acid 94. In further preferred embodiments, the present invention provides a polynucleotide encoding a protein comprising a fragment of the amino acid sequence of SEQ ID NO:190 having biological activity, the fragment preferably comprising eight (more preferably twenty, most preferably thirty) contiguous amino acids of SEQ ID NO:190, or a polynucleotide encoding a protein comprising a fragment of the amino acid sequence of SEQ ID NO:190 having biological activity, the fragment comprising the amino acid sequence from amino acid 45 to amino acid 54 of SEQ ID NO:190.

Other embodiments provide the gene corresponding to the cDNA sequence of SEQ ID NO:189.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:189, but excluding the poly(A) tail at the 3′ end of SEQ ID NO:189; and
      (ab) the nucleotide sequence of the cDNA insert of clone H438_{—}1 deposited with the ATCC under accession number 98140;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:189, but excluding the poly(A) tail at the 3′ end of SEQ ID NO:189; and
      (bb) the nucleotide sequence of the cDNA insert of clone H438_{—}1 deposited with the ATCC under accession number 98140;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:189, and extending contiguously from a nucleotide sequence corresponding to the 5′ end of SEQ ID NO:189 to a nucleotide sequence corresponding to the 3′ end of SEQ ID NO:189, but excluding the poly(A) tail at the 3′ end of SEQ ID NO:189. Also preferably the polynucleotide isolated according to the above process comprises a nucleotide sequence corresponding to the cDNA sequence of SEQ ID NO:189 from nucleotide 69 to nucleotide 371, and extending contiguously from a nucleotide sequence corresponding to the 5′ end of said sequence of SEQ ID NO:189 from nucleotide 69 to nucleotide 371, to a nucleotide sequence corresponding to the 3′ end of said sequence of SEQ ID NO:189 from nucleotide 69 to nucleotide 371. Also preferably the polynucleotide isolated according to the above process comprises a nucleotide sequence corresponding to the cDNA sequence of SEQ ID NO:189 from nucleotide 109 to nucleotide 350, and extending contiguously from a nucleotide sequence corresponding to the 5′ end of said sequence of SEQ ID NO:189 from nucleotide 109 to nucleotide 350, to a nucleotide sequence corresponding to the 3′ end of said sequence of SEQ ID NO:189 from nucleotide 109 to nucleotide 350.

In other embodiments, the present invention provides a composition comprising a protein, wherein said protein comprises an amino acid sequence selected from the group consisting of:
(a) the amino acid sequence of SEQ ID NO:190;
(b) the amino acid sequence of SEQ ID NO:190 from amino acid 17 to amino acid 94;
(c) a fragment of the amino acid sequence of SEQ ID NO:190, the fragment comprising eight contiguous amino acids of SEQ ID NO:190; and
(d) the amino acid sequence encoded by the cDNA insert of clone H438_{—}1 deposited with the ATCC under accession number 98140; the protein being substantially free from other mammalian proteins. Preferably such protein comprises the amino acid sequence of SEQ ID NO:190 or the amino acid sequence of SEQ ID NO:190 from amino acid 17 to amino acid 94. In further preferred embodiments, the present invention provides a protein comprising a fragment of the amino acid sequence of SEQ ID NO:190 having biological activity, the fragment preferably comprising eight (more preferably twenty, most preferably thirty) contiguous amino acids of SEQ ID NO:190, or a protein comprising a fragment of the amino acid sequence of SEQ ID NO:190 having biological activity, the fragment comprising the amino acid sequence from amino acid 45 to amino acid 54 of SEQ ID NO:190.

In one embodiment, the present invention provides a composition comprising an isolated polynucleotide selected from the group consisting of:
(a) a polynucleotide comprising the nucleotide sequence of SEQ ID NO:191;
(b) a polynucleotide comprising the nucleotide sequence of SEQ ID NO:191 from nucleotide 54 to nucleotide 1283;
(c) a polynucleotide comprising the nucleotide sequence of SEQ ID NO:191 from nucleotide 648 to nucleotide 1283;
(d) a polynucleotide comprising the nucleotide sequence of SEQ ID NO:191 from nucleotide 458 to nucleotide 947;
(e) a polynucleotide comprising the nucleotide sequence of the full-length protein coding sequence of clone AY421_{—}2 deposited with the ATCC under accession number 98145;
(f) a polynucleotide encoding the full-length protein encoded by the cDNA insert of clone AY421_{—}2 deposited with the ATCC under accession number 98145;
(g) a polynucleotide comprising the nucleotide sequence of a mature protein coding sequence of clone AY421_{—}2 deposited with the ATCC under accession number 98145;
(h) a polynucleotide encoding a mature protein encoded by the cDNA insert of clone AY421_{—}2 deposited with the ATCC under accession number 98145;
(i) a polynucleotide encoding a protein comprising the amino acid sequence of SEQ ID NO:192;
(j) a polynucleotide encoding a protein comprising a fragment of the amino acid sequence of SEQ ID NO:192 having biological activity, the fragment comprising eight contiguous amino acids of SEQ ID NO:192;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:191.

Preferably, such polynucleotide comprises the nucleotide sequence of SEQ ID NO:191 from nucleotide 54 to nucleotide 1283; the nucleotide sequence of SEQ ID NO:191 from nucleotide 648 to nucleotide 1283; the nucleotide sequence of SEQ ID NO:191 from nucleotide 458 to nucleotide 947; the nucleotide sequence of the full-length protein coding sequence of clone AY421_{—}2 deposited with the ATCC under accession number 98145; or the nucleotide sequence of a mature protein coding sequence of clone AY421_{—}2 deposited with the ATCC under accession number 98145. In other preferred embodiments, the polynucleotide encodes the full-length or a mature protein encoded by the cDNA insert of clone AY421_{—}2 deposited with the ATCC under accession number 98145. In yet other preferred embodiments, the present invention provides a polynucleotide encoding a protein comprising the amino acid sequence of SEQ ID NO:192 from amino acid 180 to amino acid 298. In further preferred embodiments, the present invention provides a polynucleotide encoding a protein comprising a fragment of the amino acid sequence of SEQ ID NO:192 having biological activity, the fragment preferably comprising eight (more preferably twenty, most preferably thirty) contiguous amino acids of SEQ ID NO:192, or a polynucleotide encoding a protein comprising a fragment of the amino acid sequence of SEQ ID NO:192 having biological activity, the fragment comprising the amino acid sequence from amino acid 200 to amino acid 209 of SEQ ID NO:192.

Other embodiments provide the gene corresponding to the cDNA sequence of SEQ ID NO:191.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:191, but excluding the poly(A) tail at the 3′ end of SEQ ID NO:191; and
      (ab) the nucleotide sequence of the cDNA insert of clone AY421_{—}2 deposited with the ATCC under accession number 98145;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:191, but excluding the poly(A) tail at the 3′ end of SEQ ID NO:191; and
      (bb) the nucleotide sequence of the cDNA insert of clone AY421_{—}2 deposited with the ATCC under accession number 98145;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:191, and extending contiguously from a nucleotide sequence corresponding to the 5′ end of SEQ ID NO:191 to a nucleotide sequence corresponding to the 3′ end of SEQ ID NO:191, but excluding the poly(A) tail at the 3′ end of SEQ ID NO:191. Also preferably the polynucleotide isolated according to the above process comprises a nucleotide sequence corresponding to the cDNA sequence of SEQ ID NO:191 from nucleotide 54 to nucleotide 1283, and extending contiguously from a nucleotide sequence corresponding to the 5′ end of said sequence of SEQ ID NO:191 from nucleotide 54 to nucleotide 1283, to a nucleotide sequence corresponding to the 3′ end of said sequence of SEQ ID NO:191 from nucleotide 54 to nucleotide 1283. Also preferably the polynucleotide isolated according to the above process comprises a nucleotide sequence corresponding to the cDNA sequence of SEQ ID NO:191 from nucleotide 648 to nucleotide 1283, and extending contiguously from a nucleotide sequence corresponding to the 5 end of said sequence of SEQ ID NO:191 from nucleotide 648 to nucleotide 1283, to a nucleotide sequence corresponding to the 3′ end of said sequence of SEQ ID NO:191 from nucleotide 648 to nucleotide 1283. Also preferably the polynucleotide isolated according to the above process comprises a nucleotide sequence corresponding to the cDNA sequence of SEQ ID NO:191 from nucleotide 458 to nucleotide 947, and extending contiguously from a nucleotide sequence corresponding to the 5′ end of said sequence of SEQ ID NO:191 from nucleotide 458 to nucleotide 947, to a nucleotide sequence corresponding to the 3′ end of said sequence of SEQ ID NO:191 from nucleotide 458 to nucleotide 947.

In other embodiments, the present invention provides a composition comprising a protein, wherein said protein comprises an amino acid sequence selected from the group consisting of:
(a) the amino acid sequence of SEQ ID NO:192;
(b) the amino acid sequence of SEQ ID NO:192 from amino acid 180 to amino acid 298;
(c) a fragment of the amino acid sequence of SEQ ID NO:192, the fragment comprising eight contiguous amino acids of SEQ ID NO:192; and
(d) the amino acid sequence encoded by the cDNA insert of clone AY421_{—}2 deposited with the ATCC under accession number 98145;
   the protein being substantially free from other mammalian proteins. Preferably such protein comprises the amino acid sequence of SEQ ID NO:192 or the amino acid sequence of SEQ ID NO:192 from amino acid 180 to amino acid 298. In further preferred embodiments, the present invention provides a protein comprising a fragment of the amino acid sequence of SEQ ID NO:192 having biological activity, the fragment preferably comprising eight (more preferably twenty, most preferably thirty) contiguous amino acids of SEQ ID NO:192, or a protein comprising a fragment of the amino acid sequence of SEQ ID NO:192 having biological activity, the fragment comprising the amino acid sequence from amino acid 200 to amino acid 209 of SEQ ID NO:192.

In one embodiment, the present invention provides a composition comprising an isolated polynucleotide selected from the group consisting of:
(a) a polynucleotide comprising the nucleotide sequence of SEQ ID NO:193;
(b) a polynucleotide comprising the nucleotide sequence of SEQ ID NO:193 from nucleotide 720 to nucleotide 974;
(c) a polynucleotide comprising the nucleotide sequence of SEQ ID NO:193 from nucleotide 715 to nucleotide 947;
(d) a polynucleotide comprising the nucleotide sequence of the full-length protein coding sequence of clone BV278_{—}2 deposited with the ATCC under accession number 98145;
(e) a polynucleotide encoding the full-length protein encoded by the cDNA insert of clone BV278_{—}2 deposited with the ATCC under accession number 98145;
(f) a polynucleotide comprising the nucleotide sequence of a mature protein coding sequence of clone BV278_{—}2 deposited with the ATCC under accession number 98145;
(g) a polynucleotide encoding a mature protein encoded by the cDNA insert of clone BV278_{—}2 deposited with the ATCC under accession number 98145;
(h) a polynucleotide encoding a protein comprising the amino acid sequence of SEQ ID NO:194;
(i) a polynucleotide encoding a protein comprising a fragment of the amino acid sequence of SEQ ID NO:194 having biological activity, the fragment comprising eight contiguous amino acids of SEQ ID NO:194;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:193.

Preferably, such polynucleotide comprises the nucleotide sequence of SEQ ID NO:193 from nucleotide 720 to nucleotide 974; the nucleotide sequence of SEQ ID NO:193 from nucleotide 715 to nucleotide 947; the nucleotide sequence of the full-length protein coding sequence of clone BV278_{—}2 deposited with the ATCC under accession number 98145; or the nucleotide sequence of a mature protein coding sequence of clone BV278_{—}2 deposited with the ATCC under accession number 98145. In other preferred embodiments, the polynucleotide encodes the full-length or a mature protein encoded by the cDNA insert of clone BV278_{—}2 deposited with the ATCC under accession number 98145. In yet other preferred embodiments, the present invention provides a polynucleotide encoding a protein comprising the amino acid sequence of SEQ ID NO:194 from amino acid 1 to amino acid 76. In further preferred embodiments, the present invention provides a polynucleotide encoding a protein comprising a fragment of the amino acid sequence of SEQ ID NO:194 having biological activity, the fragment preferably comprising eight (more preferably twenty, most preferably thirty) contiguous amino acids of SEQ ID NO:194, or a polynucleotide encoding a protein comprising a fragment of the amino acid sequence of SEQ ID NO:194 having biological activity, the fragment comprising the amino acid sequence from amino acid 37 to amino acid 46 of SEQ ID NO:194.

Other embodiments provide the gene corresponding to the cDNA sequence of SEQ ID NO:193.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO: 193, but excluding the poly(A) tail at the 3′ end of SEQ ID NO:193; and
      (ab) the nucleotide sequence of the cDNA insert of clone BV278_{—}2 deposited with the ATCC under accession number 98145;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:193, but excluding the poly(A) tail at the 3′ end of SEQ ID NO:193; and
      (bb) the nucleotide sequence of the cDNA insert of clone BV278_{—}2 deposited with the ATCC under accession number 98145;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:193, and extending contiguously from a nucleotide sequence corresponding to the 5′ end of SEQ ID NO:193 to a nucleotide sequence corresponding to the 3′ end of SEQ ID NO:193, but excluding the poly(A) tail at the 3′ end of SEQ ID NO:193. Also preferably the polynucleotide isolated according to the above process comprises a nucleotide sequence corresponding to the cDNA sequence of SEQ ID NO:193 from nucleotide 720 to nucleotide 974, and extending contiguously from a nucleotide sequence corresponding to the 5′ end of said sequence of SEQ ID NO:193 from nucleotide 720 to nucleotide 974, to a nucleotide sequence corresponding to the 3′ end of said sequence of SEQ ID NO:193 from nucleotide 720 to nucleotide 974. Also preferably the polynucleotide isolated according to the above process comprises a nucleotide sequence corresponding to the cDNA sequence of SEQ ID NO:193 from nucleotide 715 to nucleotide 947, and extending contiguously from a nucleotide sequence corresponding to the 5′ end of said sequence of SEQ ID NO:193 from nucleotide 715 to nucleotide 947, to a nucleotide sequence corresponding to the 3′ end of said sequence of SEQ ID NO:193 from nucleotide 715 to nucleotide 947.

In other embodiments, the present invention provides a composition comprising a protein, wherein said protein comprises an amino acid sequence selected from the group consisting of:
(a) the amino acid sequence of SEQ ID NO:194;
(b) the amino acid sequence of SEQ ID NO:194 from amino acid 1 to amino acid 76;
(c) a fragment of the amino acid sequence of SEQ ID NO:194, the fragment comprising eight contiguous amino acids of SEQ ID NO:194; and
(d) the amino acid sequence encoded by the cDNA insert of clone BV278_{—}2 deposited with the ATCC under accession number 98145;
   the protein being substantially free from other mammalian proteins. Preferably such protein comprises the amino acid sequence of SEQ ID NO:194 or the amino acid sequence of SEQ ID NO:194 from amino acid 1 to amino acid 76. In further preferred embodiments, the present invention provides a protein comprising a fragment of the amino acid sequence of SEQ ID NO:194 having biological activity, the fragment preferably comprising eight (more preferably twenty, most preferably thirty) contiguous amino acids of SEQ ID NO:194, or a protein comprising a fragment of the amino acid sequence of SEQ ID NO:194 having biological activity, the fragment comprising the amino acid sequence from amino acid 37 to amino acid 46 of SEQ ID NO:194.

In one embodiment, the present invention provides a composition comprising an isolated polynucleotide selected from the group consisting of:
(a) a polynucleotide comprising the nucleotide sequence of SEQ ID NO:195;
(b) a polynucleotide comprising the nucleotide sequence of SEQ ID NO:195 from nucleotide 36 to nucleotide 968;
(c) a polynucleotide comprising the nucleotide sequence of SEQ ID NO:195 from nucleotide 340 to nucleotide 717;
(d) a polynucleotide comprising the nucleotide sequence of the full-length protein coding sequence of clone C544_{—}1 deposited with the ATCC under accession number 98145;
(e) a polynucleotide encoding the full-length protein encoded by the cDNA insert of clone C544_{—}1 deposited with the ATCC under accession number 98145;
(f) a polynucleotide comprising the nucleotide sequence of a mature protein coding sequence of clone C544_{—}1 deposited with the ATCC under accession number 98145;
(g) a polynucleotide encoding a mature protein encoded by the cDNA insert of clone C544_{—}1 deposited with the ATCC under accession number 98145;
(h) a polynucleotide encoding a protein comprising the amino acid sequence of SEQ ID NO:196;
(i) a polynucleotide encoding a protein comprising a fragment of the amino acid sequence of SEQ ID NO:196 having biological activity, the fragment comprising eight contiguous amino acids of SEQ ID NO:196;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:195.

Preferably, such polynucleotide comprises the nucleotide sequence of SEQ ID NO:195 from nucleotide 36 to nucleotide 968; the nucleotide sequence of SEQ ID NO:195 from nucleotide 340 to nucleotide 717; the nucleotide sequence of the full-length protein coding sequence of clone C544_{—}1 deposited with the ATCC under accession number 98145; or the nucleotide sequence of a mature protein coding sequence of clone C544_{—}1 deposited with the ATCC under accession number 98145. In other preferred embodiments, the polynucleotide encodes the full-length or a mature protein encoded by the cDNA insert of clone C544_{—}1 deposited with the ATCC under accession number 98145. In yet other preferred embodiments, the present invention provides a polynucleotide encoding a protein comprising the amino acid sequence of SEQ ID NO:196 from amino acid 103 to amino acid 227. In further preferred embodiments, the present invention provides a polynucleotide encoding a protein comprising a fragment of the amino acid sequence of SEQ ID NO:196 having biological activity, the fragment preferably comprising eight (more preferably twenty, most preferably thirty) contiguous amino acids of SEQ ID NO:196, or a polynucleotide encoding a protein comprising a fragment of the amino acid sequence of SEQ ID NO:196 having biological activity, the fragment comprising the amino acid sequence from amino acid 150 to amino acid 159 of SEQ ID NO:196.

Other embodiments provide the gene corresponding to the cDNA sequence of SEQ ID NO:195.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:195, but excluding the poly(A) tail at the 3′ end of SEQ ID NO:195; and
      (ab) the nucleotide sequence of the cDNA insert of clone C544_{—}1 deposited with the ATCC under accession number 98145;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:195, but excluding the poly(A) tail at the 3′ end of SEQ ID NO:195; and
      (bb) the nucleotide sequence of the cDNA insert of clone C544_{—}1 deposited with the ATCC under accession number 98145;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:195, and extending contiguously from a nucleotide sequence corresponding to the 5′ end of SEQ ID NO:195 to a nucleotide sequence corresponding to the 3′ end of SEQ ID NO:195, but excluding the poly(A) tail at the 3′ end of SEQ ID NO:195. Also preferably the polynucleotide isolated according to the above process comprises a nucleotide sequence corresponding to the cDNA sequence of SEQ ID NO:195 from nucleotide 36 to nucleotide 968, and extending contiguously from a nucleotide sequence corresponding to the 5′ end of said sequence of SEQ ID NO:195 from nucleotide 36 to nucleotide 968, to a nucleotide sequence corresponding to the 3′ end of said sequence of SEQ ID NO:195 from nucleotide 36 to nucleotide 968. Also preferably the polynucleotide isolated according to the above process comprises a nucleotide sequence corresponding to the cDNA sequence of SEQ ID NO:195 from nucleotide 340 to nucleotide 717, and extending contiguously from a nucleotide sequence corresponding to the 5′ end of said sequence of SEQ ID NO:195 from nucleotide 340 to nucleotide 717, to a nucleotide sequence corresponding to the 3′ end of said sequence of SEQ ID NO:195 from nucleotide 340 to nucleotide 717.

In other embodiments, the present invention provides a composition comprising a protein, wherein said protein comprises an amino acid sequence selected from the group consisting of:
(a) the amino acid sequence of SEQ ID NO:196;
(b) the amino acid sequence of SEQ ID NO:196 from amino acid 103 to amino acid 227;
(c) a fragment of the amino acid sequence of SEQ ID NO:196, the fragment comprising eight contiguous amino acids of SEQ ID NO:196; and
(d) the amino acid sequence encoded by the cDNA insert of clone C544_{—}1 deposited with the ATCC under accession number 98145;
   the protein being substantially free from other mammalian proteins. Preferably such protein comprises the amino acid sequence of SEQ ID NO:196 or the amino acid sequence of SEQ ID NO:196 from amino acid 103 to amino acid 227. In further preferred embodiments, the present invention provides a protein comprising a fragment of the amino acid sequence of SEQ ID NO:196 having biological activity, the fragment preferably comprising eight (more preferably twenty, most preferably thirty) contiguous amino acids of SEQ ID NO:196, or a protein comprising a fragment of the amino acid sequence of SEQ ID NO:196 having biological activity, the fragment comprising the amino acid sequence from amino acid 150 to amino acid 159 of SEQ ID NO:196.

In one embodiment, the present invention provides a composition comprising an isolated polynucleotide selected from the group consisting of:
(a) a polynucleotide comprising the nucleotide sequence of SEQ ID NO:197;
(b) a polynucleotide comprising the nucleotide sequence of SEQ ID NO:197 from nucleotide 151 to nucleotide 1398;
(c) a polynucleotide comprising the nucleotide sequence of SEQ ID NO:197 from nucleotide 637 to nucleotide 1398;
(d) a polynucleotide comprising the nucleotide sequence of SEQ ID NO:197 from nucleotide 255 to nucleotide 429;
(e) a polynucleotide comprising the nucleotide sequence of the full-length protein coding sequence of clone CC332_{—}33 deposited with the ATCC under accession number 98145;
(f) a polynucleotide encoding the full-length protein encoded by the cDNA insert of clone CC332_{—}33 deposited with the ATCC under accession number 98145;
(g) a polynucleotide comprising the nucleotide sequence of a mature protein coding sequence of clone CC332_{—}33 deposited with the ATCC under accession number 98145;
(h) a polynucleotide encoding a mature protein encoded by the cDNA insert of clone CC332_{—}33 deposited with the ATCC under accession number 98145;
(i) a polynucleotide encoding a protein comprising the amino acid sequence of SEQ ID NO:198;
(j) a polynucleotide encoding a protein comprising a fragment of the amino acid sequence of SEQ ID NO:198 having biological activity, the fragment comprising eight contiguous amino acids of SEQ ID NO:198;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:197.

Preferably, such polynucleotide comprises the nucleotide sequence of SEQ ID NO:197 from nucleotide 151 to nucleotide 1398; the nucleotide sequence of SEQ ID NO:197 from nucleotide 637 to nucleotide 1398; the nucleotide sequence of SEQ ID NO:197 from nucleotide 255 to nucleotide 429; the nucleotide sequence of the full-length protein coding sequence of clone CC332_{—}33 deposited with the ATCC under accession number 98145; or the nucleotide sequence of a mature protein coding sequence of clone CC332_{—}33 deposited with the ATCC under accession number 98145. In other preferred embodiments, the polynucleotide encodes the full-length or a mature protein encoded by the cDNA insert of clone CC332_{—}33 deposited with the ATCC under accession number 98145. In yet other preferred embodiments, the present invention provides a polynucleotide encoding a protein comprising the amino acid sequence of SEQ ID NO:198 from amino acid 36 to amino acid 93. In further preferred embodiments, the present invention provides a polynucleotide encoding a protein comprising a fragment of the amino acid sequence of SEQ ID NO:198 having biological activity, the fragment preferably comprising eight (more preferably twenty, most preferably thirty) contiguous amino acids of SEQ ID NO:198, or a polynucleotide encoding a protein comprising a fragment of the amino acid sequence of SEQ ID NO:198 having biological activity, the fragment comprising the amino acid sequence from amino acid 203 to amino acid 212 of SEQ ID NO:198.

Other embodiments provide the gene corresponding to the cDNA sequence of SEQ ID NO:197.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:197, but excluding the poly(A) tail at the 3′ end of SEQ ID NO:197; and
      (ab) the nucleotide sequence of the cDNA insert of clone CC332_{—}33 deposited with the ATCC under accession number 98145;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:197, but excluding the poly(A) tail at the 3′ end of SEQ ID NO:197; and
      (bb) the nucleotide sequence of the cDNA insert of clone CC332_{—}33 deposited with the ATCC under accession number 98145;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:197, and extending contiguously from a nucleotide sequence corresponding to the 5′ end of SEQ ID NO:197 to a nucleotide sequence corresponding to the 3′ end of SEQ ID NO:197, but excluding the poly(A) tail at the 3′ end of SEQ ID NO:197. Also preferably the polynucleotide isolated according to the above process comprises a nucleotide sequence corresponding to the cDNA sequence of SEQ ID NO:197 from nucleotide 151 to nucleotide 1398, and extending contiguously from a nucleotide sequence corresponding to the 5′ end of said sequence of SEQ ID NO:197 from nucleotide 151 to nucleotide 1398, to a nucleotide sequence corresponding to the 3′ end of said sequence of SEQ ID NO:197 from nucleotide 151 to nucleotide 1398. Also preferably the polynucleotide isolated according to the above process comprises a nucleotide sequence corresponding to the cDNA sequence of SEQ ID NO:197 from nucleotide 637 to nucleotide 1398, and extending contiguously from a nucleotide sequence corresponding to the 5′ end of said sequence of SEQ ID NO:197 from nucleotide 637 to nucleotide 1398, to a nucleotide sequence corresponding to the 3′ end of said sequence of SEQ ID NO:197 from nucleotide 637 to nucleotide 1398. Also preferably the polynucleotide isolated according to the above process comprises a nucleotide sequence corresponding to the cDNA sequence of SEQ ID NO:197 from nucleotide 255 to nucleotide 429, and extending contiguously from a nucleotide sequence corresponding to the 5′ end of said sequence of SEQ ID NO:197 from nucleotide 255 to nucleotide 429, to a nucleotide sequence corresponding to the 3′ end of said sequence of SEQ ID NO:197 from nucleotide 255 to nucleotide 429.

In other embodiments, the present invention provides a composition comprising a protein, wherein said protein comprises an amino acid sequence selected from the group consisting of:
(a) the amino acid sequence of SEQ ID NO:198;
(b) the amino acid sequence of SEQ ID NO:198 from amino acid 36 to amino acid 93;
(c) a fragment of the amino acid sequence of SEQ ID NO:198, the fragment comprising eight contiguous amino acids of SEQ ID NO:198; and
(d) the amino acid sequence encoded by the cDNA insert of clone CC332_{—}33 deposited with the ATCC under accession number 98145;
   the protein being substantially free from other mammalian proteins. Preferably such protein comprises the amino acid sequence of SEQ ID NO:198 or the amino acid sequence of SEQ ID NO:198 from amino acid 36 to amino acid 93. In further preferred embodiments, the present invention provides a protein comprising a fragment of the amino acid sequence of SEQ ID NO:198 having biological activity, the fragment preferably comprising eight (more preferably twenty, most preferably thirty) contiguous amino acids of SEQ ID NO:198, or a protein comprising a fragment of the amino acid sequence of SEQ ID NO:198 having biological activity, the fragment comprising the amino acid sequence from amino acid 203 to amino acid 212 of SEQ ID NO:198.

In one embodiment, the present invention provides a composition comprising an isolated polynucleotide selected from the group consisting of:
(a) a polynucleotide comprising the nucleotide sequence of SEQ ID NO:199;
(b) a polynucleotide comprising the nucleotide sequence of SEQ ID NO:199 from nucleotide 21 to nucleotide 410;
(c) a polynucleotide comprising the nucleotide sequence of SEQ ID NO:199 from nucleotide 144 to nucleotide 410;
(d) a polynucleotide comprising the nucleotide sequence of SEQ ID NO:199 from nucleotide 68 to nucleotide 368;
(e) a polynucleotide comprising the nucleotide sequence of the full-length protein coding sequence of clone CC365_{—}40 deposited with the ATCC under accession number 98145;
(f) a polynucleotide encoding the full-length protein encoded by the cDNA insert of clone CC365_{—}40 deposited with the ATCC under accession number 98145;
(g) a polynucleotide comprising the nucleotide sequence of a mature protein coding sequence of clone CC365_{—}40 deposited with the ATCC under accession number 98145;
(h) a polynucleotide encoding a mature protein encoded by the cDNA insert of clone CC365_{—}40 deposited with the ATCC under accession number 98145;
(i) a polynucleotide encoding a protein comprising the amino acid sequence of SEQ ID NO:200;
(j) a polynucleotide encoding a protein comprising a fragment of the amino acid sequence of SEQ ID NO:200 having biological activity, the fragment comprising eight contiguous amino acids of SEQ ID NO:200;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:199.

Preferably, such polynucleotide comprises the nucleotide sequence of SEQ ID NO:199 from nucleotide 21 to nucleotide 410; the nucleotide sequence of SEQ ID NO:199 from nucleotide 144 to nucleotide 410; the nucleotide sequence of SEQ ID NO:199 from nucleotide 68 to nucleotide 368; the nucleotide sequence of the full-length protein coding sequence of clone CC365_{—}40 deposited with the ATCC under accession number 98145; or the nucleotide sequence of a mature protein coding sequence of clone CC365_{—}40 deposited with the ATCC under accession number 98145. In other preferred embodiments, the polynucleotide encodes the full-length or a mature protein encoded by the cDNA insert of clone CC365_{—}40 deposited with the ATCC under accession number 98145. In yet other preferred embodiments, the present invention provides a polynucleotide encoding a protein comprising the amino acid sequence of SEQ ID NO:200 from amino acid 17 to amino acid 116. In further preferred embodiments, the present invention provides a polynucleotide encoding a protein comprising a fragment of the amino acid sequence of SEQ ID NO:200 having biological activity, the fragment preferably comprising eight (more preferably twenty, most preferably thirty) contiguous amino acids of SEQ ID NO:200, or a polynucleotide encoding a protein comprising a fragment of the amino acid sequence of SEQ ID NO:200 having biological activity, the fragment comprising the amino acid sequence from amino acid 60 to amino acid 69 of SEQ ID NO:200.

Other embodiments provide the gene corresponding to the cDNA sequence of SEQ ID NO:199.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:199, but excluding the poly(A) tail at the 3′ end of SEQ ID NO:199; and
      (ab) the nucleotide sequence of the cDNA insert of clone CC365_{—}40 deposited with the ATCC under accession number 98145;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:199, but excluding the poly(A) tail at the 3′ end of SEQ ID NO:199; and
      (bb) the nucleotide sequence of the cDNA insert of clone CC365_{—}40 deposited with the ATCC under accession number 98145;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:199, and extending contiguously from a nucleotide sequence corresponding to the 5′ end of SEQ ID NO:199 to a nucleotide sequence corresponding to the 3′ end of SEQ ID NO:199, but excluding the poly(A) tail at the 3′ end of SEQ ID NO:199. Also preferably the polynucleotide isolated according to the above process comprises a nucleotide sequence corresponding to the cDNA sequence of SEQ ID NO:199 from nucleotide 21 to nucleotide 410, and extending contiguously from a nucleotide sequence corresponding to the 5′ end of said sequence of SEQ ID NO:199 from nucleotide 21 to nucleotide 410, to a nucleotide sequence corresponding to the 3′ end of said sequence of SEQ ID NO:199 from nucleotide 21 to nucleotide 410. Also preferably the polynucleotide isolated according to the above process comprises a nucleotide sequence corresponding to the cDNA sequence of SEQ ID NO:199 from nucleotide 144 to nucleotide 410, and extending contiguously from a nucleotide sequence corresponding to the 5′ end of said sequence of SEQ ID NO:199 from nucleotide 144 to nucleotide 410, to a nucleotide sequence corresponding to the 3′ end of said sequence of SEQ ID NO:199 from nucleotide 144 to nucleotide 410. Also preferably the polynucleotide isolated according to the above process comprises a nucleotide sequence corresponding to the cDNA sequence of SEQ ID NO:199 from nucleotide 68 to nucleotide 368, and extending contiguously from a nucleotide sequence corresponding to the 5′ end of said sequence of SEQ ID NO:199 from nucleotide 68 to nucleotide 368, to a nucleotide sequence corresponding to the 3′ end of said sequence of SEQ ID NO:199 from nucleotide 68 to nucleotide 368.

In other embodiments, the present invention provides a composition comprising a protein, wherein said protein comprises an amino acid sequence selected from the group consisting of:
(a) the amino acid sequence of SEQ ID NO:200;
(b) the amino acid sequence of SEQ ID NO:200 from amino acid 17 to amino acid 116;
(c) a fragment of the amino acid sequence of SEQ ID NO:200, the fragment comprising eight contiguous amino acids of SEQ ID NO:200; and
(d) the amino acid sequence encoded by the cDNA insert of clone CC365_{—}40 deposited with the ATCC under accession number 98145;
   the protein being substantially free from other mammalian proteins. Preferably such protein comprises the amino acid sequence of SEQ ID NO:200 or the amino acid sequence of SEQ ID NO:200 from amino acid 17 to amino acid 116. In further preferred embodiments, the present invention provides a protein comprising a fragment of the amino acid sequence of SEQ ID NO:200 having biological activity, the fragment preferably comprising eight (more preferably twenty, most preferably thirty) contiguous amino acids of SEQ ID NO:200, or a protein comprising a fragment of the amino acid sequence of SEQ ID NO:200 having biological activity, the fragment comprising the amino acid sequence from amino acid 60 to amino acid 69 of SEQ ID NO:200.

In one embodiment, the present invention provides a composition comprising an isolated polynucleotide selected from the group consisting of:
(a) a polynucleotide comprising the nucleotide sequence of SEQ ID NO:201;
(b) a polynucleotide comprising the nucleotide sequence of SEQ ID NO:201 from nucleotide 85 to nucleotide 966;
(c) a polynucleotide comprising the nucleotide sequence of SEQ ID NO:201 from nucleotide 127 to nucleotide 966;
(d) a polynucleotide comprising the nucleotide sequence of SEQ ID NO:201 from nucleotide 676 to nucleotide 1055;
(e) a polynucleotide comprising the nucleotide sequence of the full-length protein coding sequence of clone CG68_{—}4 deposited with the ATCC under accession number 98145;
(f) a polynucleotide encoding the full-length protein encoded by the cDNA insert of clone CG68_{—}4 deposited with the ATCC under accession number 98145;
(g) a polynucleotide comprising the nucleotide sequence of a mature protein coding sequence of clone CG68_{—}4 deposited with the ATCC under accession number 98145;
(h) a polynucleotide encoding a mature protein encoded by the cDNA insert of clone CG68_{—}4 deposited with the ATCC under accession number 98145;
(i) a polynucleotide encoding a protein comprising the amino acid sequence of SEQ ID NO:202;
(j) a polynucleotide encoding a protein comprising a fragment of the amino acid sequence of SEQ ID NO:202 having biological activity, the fragment comprising eight contiguous amino acids of SEQ ID NO:202;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:201.

Preferably, such polynucleotide comprises the nucleotide sequence of SEQ ID NO:201 from nucleotide 85 to nucleotide 966; the nucleotide sequence of SEQ ID NO:201 from nucleotide 127 to nucleotide 966; the nucleotide sequence of SEQ ID NO:201 from nucleotide 676 to nucleotide 1055; the nucleotide sequence of the full-length protein coding sequence of clone CG68_{—}4 deposited with the ATCC under accession number 98145; or the nucleotide sequence of a mature protein coding sequence of clone CG68_{—}4 deposited with the ATCC under accession number 98145. In other preferred embodiments, the polynucleotide encodes the full-length or a mature protein encoded by the cDNA insert of clone CG68_{—}4 deposited with the ATCC under accession number 98145. In yet other preferred embodiments, the present invention provides a polynucleotide encoding a protein comprising the amino acid sequence of SEQ ID NO:202 from amino acid 229 to amino acid 294. In further preferred embodiments, the present invention provides a polynucleotide encoding a protein comprising a fragment of the amino acid sequence of SEQ ID NO:202 having biological activity, the fragment preferably comprising eight (more preferably twenty, most preferably thirty) contiguous amino acids of SEQ ID NO:202, or a polynucleotide encoding a protein comprising a fragment of the amino acid sequence of SEQ ID NO:202 having biological activity, the fragment comprising the amino acid sequence from amino acid 142 to amino acid 151 of SEQ ID NO:202.

Other embodiments provide the gene corresponding to the cDNA sequence of SEQ ID NO:201.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:201, but excluding the poly(A) tail at the 3′ end of SEQ ID NO:201; and
      (ab) the nucleotide sequence of the cDNA insert of clone CG68_{—}4 deposited with the ATCC under accession number 98145;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:201, but excluding the poly(A) tail at the 3′ end of SEQ ID NO:201; and
      (bb) the nucleotide sequence of the cDNA insert of clone CG68_{—}4 deposited with the ATCC under accession number 98145;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:201, and extending contiguously from a nucleotide sequence corresponding to the 5′ end of SEQ ID NO:201 to a nucleotide sequence corresponding to the 3′ end of SEQ ID NO:201, but excluding the poly(A) tail at the 3′ end of SEQ ID NO:201. Also preferably the polynucleotide isolated according to the above process comprises a nucleotide sequence corresponding to the cDNA sequence of SEQ ID NO:201 from nucleotide 85 to nucleotide 966, and extending contiguously from a nucleotide sequence corresponding to the 5′ end of said sequence of SEQ ID NO:201 from nucleotide 85 to nucleotide 966, to a nucleotide sequence corresponding to the 3′ end of said sequence of SEQ ID NO:201 from nucleotide 85 to nucleotide 966. Also preferably the polynucleotide isolated according to the above process comprises a nucleotide sequence corresponding to the cDNA sequence of SEQ ID NO:201 from nucleotide 127 to nucleotide 966, and extending contiguously from a nucleotide sequence corresponding to the 5′ end of said sequence of SEQ ID NO:201 from nucleotide 127 to nucleotide 966, to a nucleotide sequence corresponding to the 3′ end of said sequence of SEQ ID NO:201 from nucleotide 127 to nucleotide 966. Also preferably the polynucleotide isolated according to the above process comprises a nucleotide sequence corresponding to the cDNA sequence of SEQ ID NO:201 from nucleotide 676 to nucleotide 1055, and extending contiguously from a nucleotide sequence corresponding to the 5′ end of said sequence of SEQ ID NO:201 from nucleotide 676 to nucleotide 1055, to a nucleotide sequence corresponding to the 3′ end of said sequence of SEQ ID NO:201 from nucleotide 676 to nucleotide 1055.

In other embodiments, the present invention provides a composition comprising a protein, wherein said protein comprises an amino acid sequence selected from the group consisting of:
(a) the amino acid sequence of SEQ ID NO:202;
(b) the amino acid sequence of SEQ ID NO:202 from amino acid 229 to amino acid 294;
(c) a fragment of the amino acid sequence of SEQ ID NO:202, the fragment comprising eight contiguous amino acids of SEQ ID NO:202; and
(d) the amino acid sequence encoded by the cDNA insert of clone CG68_{—}4 deposited with the ATCC under accession number 98145;
   the protein being substantially free from other mammalian proteins. Preferably such protein comprises the amino acid sequence of SEQ TD NO:202 or the amino acid sequence of SEQ ID NO:202 from amino acid 229 to amino acid 294. In further preferred embodiments, the present invention provides a protein comprising a fragment of the amino acid sequence of SEQ ID NO:202 having biological activity, the fragment preferably comprising eight (more preferably twenty, most preferably thirty) contiguous amino acids of SEQ ID NO:202, or a protein comprising a fragment of the amino acid sequence of SEQ ID NO:202 having biological activity, the fragment comprising the amino acid sequence from amino acid 142 to amino acid 151 of SEQ ID NO:202.

In one embodiment, the present invention provides a composition comprising an isolated polynucleotide selected from the group consisting of:
(a) a polynucleotide comprising the nucleotide sequence of SEQ ID NO:203;
(b) a polynucleotide comprising the nucleotide sequence of SEQ ID NO:203 from nucleotide 515 to nucleotide 1633;
(c) a polynucleotide comprising the nucleotide sequence of SEQ ID NO:203 from nucleotide 758 to nucleotide 1633;
(d) a polynucleotide comprising the nucleotide sequence of SEQ ID NO:203 from nucleotide 1088 to nucleotide 1572;
(e) a polynucleotide comprising the nucleotide sequence of the full-length protein coding sequence of clone D329_{—}1 deposited with the ATCC under accession number 98145;
(f) a polynucleotide encoding the full-length protein encoded by the cDNA insert of clone D329_{—}1 deposited with the ATCC under accession number 98145;
(g) a polynucleotide comprising the nucleotide sequence of a mature protein coding sequence of clone D329_{—}1 deposited with the ATCC under accession number 98145;
(h) a polynucleotide encoding a mature protein encoded by the cDNA insert of clone D329_{—}1 deposited with the ATCC under accession number 98145;
(i) a polynucleotide encoding a protein comprising the amino acid sequence of SEQ ID NO:204;
(j) a polynucleotide encoding a protein comprising a fragment of the amino acid sequence of SEQ ID NO:204 having biological activity, the fragment comprising eight contiguous amino acids of SEQ ID NO:204;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:203.

Preferably, such polynucleotide comprises the nucleotide sequence of SEQ ID NO:203 from nucleotide 515 to nucleotide 1633; the nucleotide sequence of SEQ ID NO:203 from nucleotide 758 to nucleotide 1633; the nucleotide sequence of SEQ ID NO:203 from nucleotide 1088 to nucleotide 1572; the nucleotide sequence of the full-length protein coding sequence of clone D329_{—}1 deposited with the ATCC under accession number 98145; or the nucleotide sequence of a mature protein coding sequence of clone D329_{—}1 deposited with the ATCC under accession number 98145. In other preferred embodiments, the polynucleotide encodes the full-length or a mature protein encoded by the cDNA insert of clone D329_{—}1 deposited with the ATCC under accession number 98145. In yet other preferred embodiments, the present invention provides a polynucleotide encoding a protein comprising the amino acid sequence of SEQ ID NO:204 from amino acid 241 to amino acid 353. In further preferred embodiments, the present invention provides a polynucleotide encoding a protein comprising a fragment of the amino acid sequence of SEQ ID NO:204 having biological activity, the fragment preferably comprising eight (more preferably twenty, most preferably thirty) contiguous amino acids of SEQ ID NO:204, or a polynucleotide encoding a protein comprising a fragment of the amino acid sequence of SEQ ID NO:204 having biological activity, the fragment comprising the amino acid sequence from amino acid 181 to amino acid 190 of SEQ ID NO:204.

Other embodiments provide the gene corresponding to the cDNA sequence of SEQ ID NO:203.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:203, but excluding the poly(A) tail at the 3′ end of SEQ ID NO:203; and
      (ab) the nucleotide sequence of the cDNA insert of clone D329_{—}1 deposited with the ATCC under accession number 98145;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:203, but excluding the poly(A) tail at the 3′ end of SEQ ID NO:203; and
      (bb) the nucleotide sequence of the cDNA insert of clone D329_{—}1 deposited with the ATCC under accession number 98145;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:203, and extending contiguously from a nucleotide sequence corresponding to the 5′ end of SEQ ID NO:203 to a nucleotide sequence corresponding to the 3′ end of SEQ ID NO:203, but excluding the poly(A) tail at the 3′ end of SEQ ID NO:203. Also preferably the polynucleotide isolated according to the above process comprises a nucleotide sequence corresponding to the cDNA sequence of SEQ ID NO:203 from nucleotide 515 to nucleotide 1633, and extending contiguously from a nucleotide sequence corresponding to the 5′ end of said sequence of SEQ ID NO:203 from nucleotide 515 to nucleotide 1633, to a nucleotide sequence corresponding to the 3′ end of said sequence of SEQ ID NO:203 from nucleotide 515 to nucleotide 1633. Also preferably the polynucleotide isolated according to the above process comprises a nucleotide sequence corresponding to the cDNA sequence of SEQ ID NO:203 from nucleotide 758 to nucleotide 1633, and extending contiguously from a nucleotide sequence corresponding to the 5′ end of said sequence of SEQ ID NO:203 from nucleotide 758 to nucleotide 1633, to a nucleotide sequence corresponding to the 3′ end of said sequence of SEQ ID NO:203 from nucleotide 758 to nucleotide 1633. Also preferably the polynucleotide isolated according to the above process comprises a nucleotide sequence corresponding to the cDNA sequence of SEQ ID NO:203 from nucleotide 1088 to nucleotide 1572, and extending contiguously from a nucleotide sequence corresponding to the 5′ end of said sequence of SEQ ID NO:203 from nucleotide 1088 to nucleotide 1572, to a nucleotide sequence corresponding to the 3′ end of said sequence of SEQ ID NO:203 from nucleotide 1088 to nucleotide 1572.

In other embodiments, the present invention provides a composition comprising a protein, wherein said protein comprises an amino acid sequence selected from the group consisting of:
(a) the amino acid sequence of SEQ ID NO:204;
(b) the amino acid sequence of SEQ ID NO:204 from amino acid 241 to amino acid 353;
(c) a fragment of the amino acid sequence of SEQ ID NO:204, the fragment comprising eight contiguous amino acids of SEQ ID NO:204; and
(d) the amino acid sequence encoded by the cDNA insert of clone D329_{—}1 deposited with the ATCC under accession number 98145;
   the protein being substantially free from other mammalian proteins. Preferably such protein comprises the amino acid sequence of SEQ ID NO:204 or the amino acid sequence of SEQ ID NO:204 from amino acid 241 to amino acid 353. In further preferred embodiments, the present invention provides a protein comprising a fragment of the amino acid sequence of SEQ ID NO:204 having biological activity, the fragment preferably comprising eight (more preferably twenty, most preferably thirty) contiguous amino acids of SEQ ID NO:204, or a protein comprising a fragment of the amino acid sequence of SEQ ID NO:204 having biological activity, the fragment comprising the amino acid sequence from amino acid 181 to amino acid 190 of SEQ ID NO:204.

In one embodiment, the present invention provides a composition comprising an isolated polynucleotide selected from the group consisting of:
(a) a polynucleotide comprising the nucleotide sequence of SEQ ID NO:206;
(b) a polynucleotide comprising the nucleotide sequence of SEQ ID NO:206 from nucleotide 279 to nucleotide 515;
(c) a polynucleotide comprising the nucleotide sequence of the full-length protein coding sequence of clone H698_{—}3 deposited with the ATCC under accession number 98145;
(d) a polynucleotide encoding the full-length protein encoded by the cDNA insert of clone H698_{—}3 deposited with the ATCC under accession number 98145;
(e) a polynucleotide comprising the nucleotide sequence of a mature protein coding sequence of clone H698_{—}3 deposited with the ATCC under accession number 98145;
(f) a polynucleotide encoding a mature protein encoded by the cDNA insert of clone H698_{—}3 deposited with the ATCC under accession number 98145;
(g) a polynucleotide encoding a protein comprising the amino acid sequence of SEQ ID NO:207;
(h) a polynucleotide encoding a protein comprising a fragment of the amino acid sequence of SEQ ID NO:207 having biological activity, the fragment comprising eight contiguous amino acids of SEQ ID NO:207;
(i) a polynucleotide which is an allelic variant of a polynucleotide of (a)-(f) above;
(j) a polynucleotide which encodes a species homologue of the protein of (g) or (h) above;
(k) a polynucleotide that hybridizes under stringent conditions to any one of the polynucleotides specified in (a)-(h); and
(l) a polynucleotide that hybridizes under stringent conditions to any one of the polynucleotides specified in (a)-(h) and that has a length that is at least 25% of the length of SEQ ID NO:206.

Preferably, such polynucleotide comprises the nucleotide sequence of SEQ ID NO:206 from nucleotide 279 to nucleotide 515; the nucleotide sequence of the full-length protein coding sequence of clone H698_{—}3 deposited with the ATCC under accession number 98145; or the nucleotide sequence of a mature protein coding sequence of clone H698_{—}3 deposited with the ATCC under accession number 98145. In other preferred embodiments, the polynucleotide encodes the full-length or a mature protein encoded by the cDNA insert of clone H698_{—}3 deposited with the ATCC under accession number 98145. In further preferred embodiments, the present invention provides a polynucleotide encoding a protein comprising a fragment of the amino acid sequence of SEQ ID NO:207 having biological activity, the fragment preferably comprising eight (more preferably twenty, most preferably thirty) contiguous amino acids of SEQ ID NO:207, or a polynucleotide encoding a protein comprising a fragment of the amino acid sequence of SEQ ID NO:207 having biological activity, the fragment comprising the amino acid sequence from amino acid 34 to amino acid 43 of SEQ ID NO:207.

Other embodiments provide the gene corresponding to the cDNA sequence of SEQ ID NO:206, SEQ ID NO:205, and SEQ ID NO:208.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:205;
      (ab) SEQ ID NO:206;
      (ac) SEQ ID NO:208, but excluding the poly(A) tail at the 3′ end of SEQ ID NO:208; and
      (ad) the nucleotide sequence of the cDNA insert of clone H698_{—}3 deposited with the ATCC under accession number 98145;
   (ii) hybridizing said probe(s) to human genomic DNA in conditions at least as stringent as 4×SSC at 50 degrees C.; and
   (ii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:205;
      (bb) SEQ ID NO:206;
      (bc) SEQ ID NO:208, but excluding the poly(A) tail at the 3′ end of SEQ ID NO:208; and
      (bd) the nucleotide sequence of the cDNA insert of clone H698_{—}3 deposited with the ATCC under accession number 98145;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequences of SEQ ID NO:205, SEQ ID NO:206, and SEQ ID NO:208, and extending contiguously from a nucleotide sequence corresponding to the 5′ end of SEQ ID NO:205 to a nucleotide sequence corresponding to the 3′ end of SEQ ID NO:208, but excluding the poly(A) tail at the 3′ end of SEQ ID NO:208. Also preferably the polynucleotide isolated according to the above process comprises a nucleotide sequence corresponding to the cDNA sequence of SEQ ID NO:206, and extending contiguously from a nucleotide sequence corresponding to the 5′ end of SEQ ID NO:206 to a nucleotide sequence corresponding to the 3′ end of SEQ ID NO:206. Also preferably the polynucleotide isolated according to the above process comprises a nucleotide sequence corresponding to the cDNA sequence of SEQ ID NO:206 from nucleotide 279 to nucleotide 515, and extending contiguously from a nucleotide sequence corresponding to the 5′ end of said sequence of SEQ ID NO:206 from nucleotide 279 to nucleotide 515, to a nucleotide sequence corresponding to the 3′ end of said sequence of SEQ ID NO:206 from nucleotide 279 to nucleotide 515.

In other embodiments, the present invention provides a composition comprising a protein, wherein said protein comprises an amino acid sequence selected from the group consisting of:
(a) the amino acid sequence of SEQ ID NO:207;
(b) a fragment of the amino acid sequence of SEQ ID NO:207, the fragment comprising eight contiguous amino acids of SEQ ID NO:207; and
(c) the amino acid sequence encoded by the cDNA insert of clone H698_{—}3 deposited with the ATCC under accession number 98145;
   the protein being substantially free from other mammalian proteins. Preferably such protein comprises the amino acid sequence of SEQ ID NO:207. In further preferred embodiments, the present invention provides a protein comprising a fragment of the amino acid sequence of SEQ ID NO:207 having biological activity, the fragment preferably comprising eight (more preferably twenty, most preferably thirty) contiguous amino acids of SEQ ID NO:207, or a protein comprising a fragment of the amino acid sequence of SEQ ID NO:207 having biological activity, the fragment comprising the amino acid sequence from amino acid 34 to amino acid 43 of SEQ ID NO:207.

In one embodiment, the present invention provides a composition comprising an isolated polynucleotide selected from the group consisting of:
(a) a polynucleotide comprising the nucleotide sequence of SEQ ID NO:209;
(b) a polynucleotide comprising the nucleotide sequence of SEQ ID NO:209 from nucleotide 199 to nucleotide 1155;
(c) a polynucleotide comprising the nucleotide sequence of SEQ ID NO:209 from nucleotide 304 to nucleotide 1155;
(d) a polynucleotide comprising the nucleotide sequence of the full-length protein coding sequence of clone H963_{—}20 deposited with the ATCC under accession number 98145;
(e) a polynucleotide encoding the full-length protein encoded by the cDNA insert of clone H963_{—}20 deposited with the ATCC under accession number 98145;
(f) a polynucleotide comprising the nucleotide sequence of a mature protein coding sequence of clone H963_{—}20 deposited with the ATCC under accession number 98145;
(g) a polynucleotide encoding a mature protein encoded by the cDNA insert of clone H963_{—}20 deposited with the ATCC under accession number 98145;
(h) a polynucleotide encoding a protein comprising the amino acid sequence of SEQ ID NO:210;
(i) a polynucleotide encoding a protein comprising a fragment of the amino acid sequence of SEQ ID NO:210 having biological activity, the fragment comprising eight contiguous amino acids of SEQ ID NO:210;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:209.

Preferably, such polynucleotide comprises the nucleotide sequence of SEQ ID NO:209 from nucleotide 199 to nucleotide 1155; the nucleotide sequence of SEQ ID NO:209 from nucleotide 304 to nucleotide 1155; the nucleotide sequence of the full-length protein coding sequence of clone H963_{—}20 deposited with the ATCC under accession number 98145; or the nucleotide sequence of a mature protein coding sequence of clone H963_{—}20 deposited with the ATCC under accession number 98145. In other preferred embodiments, the polynucleotide encodes the full-length or a mature protein encoded by the cDNA insert of clone H963_{—}20 deposited with the ATCC under accession number 98145. In yet other preferred embodiments, the present invention provides a polynucleotide encoding a protein comprising the amino acid sequence of SEQ ID NO:210 from amino acid 19 to amino acid 84. In further preferred embodiments, the present invention provides a polynucleotide encoding a protein comprising a fragment of the amino acid sequence of SEQ ID NO:210 having biological activity, the fragment preferably comprising eight (more preferably twenty, most preferably thirty) contiguous amino acids of SEQ ID NO:210, or a polynucleotide encoding a protein comprising a fragment of the amino acid sequence of SEQ ID NO:210 having biological activity, the fragment comprising the amino acid sequence from amino acid 154 to amino acid 163 of SEQ ID NO:210.

Other embodiments provide the gene corresponding to the cDNA sequence of SEQ ID NO:209.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:209, but excluding the poly(A) tail at the 3′ end of SEQ ID NO:209; and
      (ab) the nucleotide sequence of the cDNA insert of clone H963_{—}20 deposited with the ATCC under accession number 98145;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:209, but excluding the poly(A) tail at the 3′ end of SEQ ID NO:209; and
      (bb) the nucleotide sequence of the cDNA insert of clone H963_{—}20 deposited with the ATCC under accession number 98145;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:209, and extending contiguously from a nucleotide sequence corresponding to the 5′ end of SEQ ID NO:209 to a nucleotide sequence corresponding to the 3′ end of SEQ ID NO:209, but excluding the poly(A) tail at the 3′ end of SEQ ID NO:209. Also preferably the polynucleotide isolated according to the above process comprises a nucleotide sequence corresponding to the cDNA sequence of SEQ ID NO:209 from nucleotide 199 to nucleotide 1155, and extending contiguously from a nucleotide sequence corresponding to the 5′ end of said sequence of SEQ ID NO:209 from nucleotide 199 to nucleotide 1155, to a nucleotide sequence corresponding to the 3′ end of said sequence of SEQ ID NO:209 from nucleotide 199 to nucleotide 1155. Also preferably the polynucleotide isolated according to the above process comprises a nucleotide sequence corresponding to the cDNA sequence of SEQ ID NO:209 from nucleotide 304 to nucleotide 1155, and extending contiguously from a nucleotide sequence corresponding to the 5′ end of said sequence of SEQ ID NO:209 from nucleotide 304 to nucleotide 1155, to a nucleotide sequence corresponding to the 3′ end of said sequence of SEQ ID NO:209 from nucleotide 304 to nucleotide 1155.

In other embodiments, the present invention provides a composition comprising a protein, wherein said protein comprises an amino acid sequence selected from the group consisting of:
(a) the amino acid sequence of SEQ ID NO:210;
(b) the amino acid sequence of SEQ ID NO:210 from amino acid 19 to amino acid 84;
(c) a fragment of the amino acid sequence of SEQ ID NO:210, the fragment comprising eight contiguous amino acids of SEQ ID NO:210; and
(d) the amino acid sequence encoded by the cDNA insert of clone H963_{—}20 deposited with the ATCC under accession number 98145;
   the protein being substantially free from other mammalian proteins. Preferably such protein comprises the amino acid sequence of SEQ ID NO:210 or the amino acid sequence of SEQ ID NO:210 from amino acid 19 to amino acid 84. In further preferred embodiments, the present invention provides a protein comprising a fragment of the amino acid sequence of SEQ ID NO:210 having biological activity, the fragment preferably comprising eight (more preferably twenty, most preferably thirty) contiguous amino acids of SEQ ID NO:210, or a protein comprising a fragment of the amino acid sequence of SEQ ID NO:210 having biological activity, the fragment comprising the amino acid sequence from amino acid 154 to amino acid 163 of SEQ ID NO:210.

In one embodiment, the present invention provides a composition comprising an isolated polynucleotide selected from the group consisting of:
(a) a polynucleotide comprising the nucleotide sequence of SEQ ID NO:211;
(b) a polynucleotide comprising the nucleotide sequence of SEQ ID NO:211 from nucleotide 247 to nucleotide 441;
(c) a polynucleotide comprising the nucleotide sequence of SEQ ID NO:211 from nucleotide 319 to nucleotide 441;
(d) a polynucleotide comprising the nucleotide sequence of SEQ ID NO:211 from nucleotide 294 to nucleotide 432;
(e) a polynucleotide comprising the nucleotide sequence of the full-length protein coding sequence of clone BD372_{—}5 deposited with the ATCC under accession number 98146;
(f) a polynucleotide encoding the full-length protein encoded by the cDNA insert of clone BD372_{—}5 deposited with the ATCC under accession number 98146;
(g) a polynucleotide comprising the nucleotide sequence of a mature protein coding sequence of clone BD372_{—}5 deposited with the ATCC under accession number 98146;
(h) a polynucleotide encoding a mature protein encoded by the cDNA insert of clone BD372_{—}5 deposited with the ATCC under accession number 98146;
(i) a polynucleotide encoding a protein comprising the amino acid sequence of SEQ ID NO:212;
(j) a polynucleotide encoding a protein comprising a fragment of the amino acid sequence of SEQ ID NO:212 having biological activity, the fragment comprising eight contiguous amino acids of SEQ ID NO:212;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:211.

Preferably, such polynucleotide comprises the nucleotide sequence of SEQ ID NO:211 from nucleotide 247 to nucleotide 441; the nucleotide sequence of SEQ ID NO:211 from nucleotide 319 to nucleotide 441; the nucleotide sequence of SEQ ID NO:211 from nucleotide 294 to nucleotide 432; the nucleotide sequence of the full-length protein coding sequence of clone BD372_{—}5 deposited with the ATCC under accession number 98146; or the nucleotide sequence of a mature protein coding sequence of clone BD372_{—}5 deposited with the ATCC under accession number 98146. In other preferred embodiments, the polynucleotide encodes the full-length or a mature protein encoded by the cDNA insert of clone BD372_{—}5 deposited with the ATCC under accession number 98146. In yet other preferred embodiments, the present invention provides a polynucleotide encoding a protein comprising the amino acid sequence of SEQ ID NO:212 from amino acid 17 to amino acid 62. In further preferred embodiments, the present invention provides a polynucleotide encoding a protein comprising a fragment of the amino acid sequence of SEQ ID NO:212 having biological activity, the fragment preferably comprising eight (more preferably twenty, most preferably thirty) contiguous amino acids of SEQ ID NO:212, or a polynucleotide encoding a protein comprising a fragment of the amino acid sequence of SEQ ID NO:212 having biological activity, the fragment comprising the amino acid sequence from amino acid 27 to amino acid 36 of SEQ ID NO:212. Other embodiments provide the gene corresponding to the cDNA sequence of SEQ ID NO:211.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:211, but excluding the poly(A) tail at the 3′ end of SEQ ID NO:211; and
      (ab) the nucleotide sequence of the cDNA insert of clone BD372_{—}5 deposited with the ATCC under accession number 98146;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:211, but excluding the poly(A) tail at the 3′ end of SEQ ID NO:211; and
      (bb) the nucleotide sequence of the cDNA insert of clone BD372_{—}5 deposited with the ATCC under accession number 98146;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:211, and extending contiguously from a nucleotide sequence corresponding to the 5′ end of SEQ ID NO:211 to a nucleotide sequence corresponding to the 3′ end of SEQ ID NO:211, but excluding the poly(A) tail at the 3′ end of SEQ ID NO:211. Also preferably the polynucleotide isolated according to the above process comprises a nucleotide sequence corresponding to the cDNA sequence of SEQ ID NO:211 from nucleotide 247 to nucleotide 441, and extending contiguously from a nucleotide sequence corresponding to the 5′ end of said sequence of SEQ ID NO:211 from nucleotide 247 to nucleotide 441, to a nucleotide sequence corresponding to the 3′ end of said sequence of SEQ ID NO:211 from nucleotide 247 to nucleotide 441. Also preferably the polynucleotide isolated according to the above process comprises a nucleotide sequence corresponding to the cDNA sequence of SEQ ID NO:211 from nucleotide 319 to nucleotide 441, and extending contiguously from a nucleotide sequence corresponding to the 5′ end of said sequence of SEQ ID NO:211 from nucleotide 319 to nucleotide 441, to a nucleotide sequence corresponding to the 3′ end of said sequence of SEQ ID NO:211 from nucleotide 319 to nucleotide 441. Also preferably the polynucleotide isolated according to the above process comprises a nucleotide sequence corresponding to the cDNA sequence of SEQ ID NO:211 from nucleotide 294 to nucleotide 432, and extending contiguously from a nucleotide sequence corresponding to the 5′ end of said sequence of SEQ ID NO:211 from nucleotide 294 to nucleotide 432, to a nucleotide sequence corresponding to the 3′ end of said sequence of SEQ ID NO:211 from nucleotide 294 to nucleotide 432.

In other embodiments, the present invention provides a composition comprising a protein, wherein said protein comprises an amino acid sequence selected from the group consisting of:
(a) the amino acid sequence of SEQ ID NO:212;
(b) the amino acid sequence of SEQ ID NO:212 from amino acid 17 to amino acid 62;
(c) a fragment of the amino acid sequence of SEQ ID NO:212, the fragment comprising eight contiguous amino acids of SEQ ID NO:212; and
(d) the amino acid sequence encoded by the cDNA insert of clone BD372_{—}5 deposited with the ATCC under accession number 98146;
   the protein being substantially free from other mammalian proteins. Preferably such protein comprises the amino acid sequence of SEQ ID NO:212 or the amino acid sequence of SEQ ID NO:212 from amino acid 17 to amino acid 62. In further preferred embodiments, the present invention provides a protein comprising a fragment of the amino acid sequence of SEQ ID NO:212 having biological activity, the fragment preferably comprising eight (more preferably twenty, most preferably thirty) contiguous amino acids of SEQ ID NO:212, or a protein comprising a fragment of the amino acid sequence of SEQ ID NO:212 having biological activity, the fragment comprising the amino acid sequence from amino acid 27 to amino acid 36 of SEQ ID NO:212.

In one embodiment, the present invention provides a composition comprising an isolated polynucleotide selected from the group consisting of:
(a) a polynucleotide comprising the nucleotide sequence of SEQ ID NO:213;
(b) a polynucleotide comprising the nucleotide sequence of SEQ ID NO:213 from nucleotide 245 to nucleotide 2497;
(c) a polynucleotide comprising the nucleotide sequence of SEQ ID NO:213 from nucleotide 299 to nucleotide 2497;
(d) a polynucleotide comprising the nucleotide sequence of SEQ ID NO:213 from nucleotide 154 to nucleotide 430;
(e) a polynucleotide comprising the nucleotide sequence of the full-length protein coding sequence of clone BR533_{—}4 deposited with the ATCC under accession number 98146;
(f) a polynucleotide encoding the full-length protein encoded by the cDNA insert of clone BR533_{—}4 deposited with the ATCC under accession number 98146;
(g) a polynucleotide comprising the nucleotide sequence of a mature protein coding sequence of clone BR533_{—}4 deposited with the ATCC under accession number 98146;
(h) a polynucleotide encoding a mature protein encoded by the cDNA insert of clone BR533_{—}4 deposited with the ATCC under accession number 98146;
(i) a polynucleotide encoding a protein comprising the amino acid sequence of SEQ ID NO:214;
(j) a polynucleotide encoding a protein comprising a fragment of the amino acid sequence of SEQ ID NO:214 having biological activity, the fragment comprising eight contiguous amino acids of SEQ ID NO:214;
(k) a polynucleotide which is an allelic variant of a polynucleotide of (a)-(h) above;
(l) a polynucleotide which encodes a species homologue of the protein of (i) or (j) above;
(m) a polynucleotide that hybridizes under stringent conditions to any one of the polynucleotides specified in (a)-(j); and
(n) a polynucleotide that hybridizes under stringent conditions to any one of the polynucleotides specified in (a)-(j) and that has a length that is at least 25% of the length of SEQ ID NO:213.

Preferably, such polynucleotide comprises the nucleotide sequence of SEQ ID NO:213 from nucleotide 245 to nucleotide 2497; the nucleotide sequence of SEQ ID NO:213 from nucleotide 299 to nucleotide 2497; the nucleotide sequence of SEQ ID NO:213 from nucleotide 154 to nucleotide 430; the nucleotide sequence of the full-length protein coding sequence of clone BR533_{—}4 deposited with the ATCC under accession number 98146; or the nucleotide sequence of a mature protein coding sequence of clone BR533_{—}4 deposited with the ATCC under accession number 98146. In other preferred embodiments, the polynucleotide encodes the full-length or a mature protein encoded by the cDNA insert of clone BR533_{—}4 deposited with the ATCC under accession number 98146. In yet other preferred embodiments, the present invention provides a polynucleotide encoding a protein comprising the amino acid sequence of SEQ ID NO:214 from amino acid 1 to amino add 62. In further preferred embodiments, the present invention provides a polynucleotide encoding a protein comprising a fragment of the amino acid sequence of SEQ ID NO:214 having biological activity, the fragment preferably comprising eight (more preferably twenty, most preferably thirty) contiguous amino acids of SEQ ID NO:214, or a polynucleotide encoding a protein comprising a fragment of the amino acid sequence of SEQ ID NO:214 having biological activity, the fragment comprising the amino add sequence from amino acid 370 to amino acid 379 of SEQ ID NO:214.

Other embodiments provide the gene corresponding to the cDNA sequence of SEQ ID NO:213.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:213, but excluding the poly(A) tail at the 3′ end of SEQ ID NO:213; and
      (ab) the nucleotide sequence of the cDNA insert of clone BR533_{—}4 deposited with the ATCC under accession number 98146;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:213, but excluding the poly(A) tail at the 3′ end of SEQ ID NO:213; and
      (bb) the nucleotide sequence of the cDNA insert of clone BR533_{—}4 deposited with the ATCC under accession number 98146;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:213, and extending contiguously from a nucleotide sequence corresponding to the 5′ end of SEQ ID NO:213 to a nucleotide sequence corresponding to the 3′ end of SEQ ID NO:213, but excluding the poly(A) tail at the 3′ end of SEQ ID NO:213. Also preferably the polynucleotide isolated according to the above process comprises a nucleotide sequence corresponding to the cDNA sequence of SEQ ID NO:213 from nucleotide 245 to nucleotide 2497, and extending contiguously from a nucleotide sequence corresponding to the 5′ end of said sequence of SEQ ID NO:213 from nucleotide 245 to nucleotide 2497, to a nucleotide sequence corresponding to the 3′ end of said sequence of SEQ ID NO:213 from nucleotide 245 to nucleotide 2497. Also preferably the polynucleotide isolated according to the above process comprises a nucleotide sequence corresponding to the cDNA sequence of SEQ ID NO:213 from nucleotide 299 to nucleotide 2497, and extending contiguously from a nucleotide sequence corresponding to the 5′ end of said sequence of SEQ ID NO:213 from nucleotide 299 to nucleotide 2497, to a nucleotide sequence corresponding to the 3′ end of said sequence of SEQ ID NO:213 from nucleotide 299 to nucleotide 2497. Also preferably the polynucleotide isolated according to the above process comprises a nucleotide sequence corresponding to the cDNA sequence of SEQ ID NO:213 from nucleotide 154 to nucleotide 430, and extending contiguously from a nucleotide sequence corresponding to the 5′ end of said sequence of SEQ ID NO:213 from nucleotide 154 to nucleotide 430, to a nucleotide sequence corresponding to the 3′ end of said sequence of SEQ ID NO:213 from nucleotide 154 to nucleotide 430.

In other embodiments, the present invention provides a composition comprising a protein, wherein said protein comprises an amino acid sequence selected from the group consisting of:
(a) the amino acid sequence of SEQ ID NO:214;
(b) the amino acid sequence of SEQ ID NO:214 from amino acid 1 to amino acid 62;
(c) a fragment of the amino acid sequence of SEQ ID NO:214, the fragment comprising eight contiguous amino acids of SEQ ID NO:214; and
(d) the amino acid sequence encoded by the cDNA insert of clone BR533_{—}4 deposited with the ATCC under accession number 98146;
   the protein being substantially free from other mammalian proteins. Preferably such protein comprises the amino acid sequence of SEQ ID NO:214 or the amino acid sequence of SEQ ID NO:214 from amino acid 1 to amino acid 62. In further preferred embodiments, the present invention provides a protein comprising a fragment of the amino acid sequence of SEQ ID NO:214 having biological activity, the fragment preferably comprising eight (more preferably twenty, most preferably thirty) contiguous amino acids of SEQ ID NO:214, or a protein comprising a fragment of the amino acid sequence of SEQ ID NO:214 having biological activity, the fragment comprising the amino acid sequence from amino acid 370 to amino acid 379 of SEQ ID NO:214.

In one embodiment, the present invention provides a composition comprising an isolated polynucleotide selected from the group consisting of:
(a) a polynucleotide comprising the nucleotide sequence of SEQ ID NO:215;
(b) a polynucleotide comprising the nucleotide sequence of SEQ ID NO:215 from nucleotide 30 to nucleotide 434;
(c) a polynucleotide comprising the nucleotide sequence of SEQ ID NO:215 from nucleotide 149 to nucleotide 447;
(d) a polynucleotide comprising the nucleotide sequence of the full-length protein coding sequence of clone CC288_{—}9 deposited with the ATCC under accession number 98146;
(e) a polynucleotide encoding the full-length protein encoded by the cDNA insert of clone CC288_{—}9 deposited with the ATCC under accession number 98146;
(f) a polynucleotide comprising the nucleotide sequence of a mature protein coding sequence of clone CC288_{—}9 deposited with the ATCC under accession number 98146;
(g) a polynucleotide encoding a mature protein encoded by the cDNA insert of clone CC288_{—}9 deposited with the ATCC under accession number 98146;
(h) a polynucleotide encoding a protein comprising the amino acid sequence of SEQ ID NO:216;
(i) a polynucleotide encoding a protein comprising a fragment of the amino acid sequence of SEQ ID NO:216 having biological activity, the fragment comprising eight contiguous amino acids of SEQ ID NO:216;
(j) a polynucleotide which is an allelic variant of a polynucleotide of (a)-(g) above;
(k) a polynucleotide which encodes a species homologue of the protein of (h) or (i) above;
(l) a polynucleotide that hybridizes under stringent conditions to any one of the polynucleotides specified in (a)-(i); and
(m) a polynucleotide that hybridizes under stringent conditions to any one of the polynucleotides specified in (a)-(i) and that has a length that is at least 25% of the length of SEQ ID NO:215.

Preferably, such polynucleotide comprises the nucleotide sequence of SEQ ID NO:215 from nucleotide 30 to nucleotide 434; the nucleotide sequence of SEQ ID NO:215 from nucleotide 149 to nucleotide 447; the nucleotide sequence of the full-length protein coding sequence of clone CC288_{—}9 deposited with the ATCC under accession number 98146; or the nucleotide sequence of a mature protein coding sequence of clone CC288_{—}9 deposited with the ATCC under accession number 98146. In other preferred embodiments, the polynucleotide encodes the full-length or a mature protein encoded by the cDNA insert of clone CC288_{—}9 deposited with the ATCC under accession number 98146. In yet other preferred embodiments, the present invention provides a polynucleotide encoding a protein comprising the amino acid sequence of SEQ ID NO:216 from amino acid 41 to amino acid 135. In further preferred embodiments, the present invention provides a polynucleotide encoding a protein comprising a fragment of the amino acid sequence of SEQ ID NO:216 having biological activity, the fragment preferably comprising eight (more preferably twenty, most preferably thirty) contiguous amino acids of SEQ ID NO:216, or a polynucleotide encoding a protein comprising a fragment of the amino acid sequence of SEQ ID NO:216 having biological activity, the fragment comprising the amino acid sequence from amino acid 62 to amino acid 71 of SEQ ID NO:216.

Other embodiments provide the gene corresponding to the cDNA sequence of SEQ ID NO:215.

Further embodiments of the invention provide isolated polynucleotides produced according to a process selected from the group consisting of:
(a) a process comprising the steps of:
   (i) preparing one or more polynucleotide probes that hybridize in 6×SSC at 65 degrees C. to a nucleotide sequence selected from the group consisting of:
      (aa) SEQ ID NO:215, but excluding the poly(A) tail at the 3′ end of SEQ ID NO:215; and
      (ab) the nucleotide sequence of the cDNA insert of clone CC288_{—}9 deposited with the ATCC under accession number 98146;
   (ii) hybridizing said probe(s) to human genomic DNA in conditions at least as stringent as 4×SSC at 50 degrees C.; and
   (iii) isolating the DNA polynucleotides detected with the probe(s);

and
(b) a process comprising the steps of:
   (i) preparing one or more polynucleotide primers that hybridize in 6×SSC at 65 degrees C. to a nucleotide sequence selected from the group consisting of:
      (ba) SEQ ID NO:215, but excluding the poly(A) tail at the 3′ end of SEQ ID NO:215; and
      (bb) the nucleotide sequence of the cDNA insert of clone CC288_{—}9 deposited with the ATCC under accession number 98146;
   (ii) hybridizing said primer(s) to human genomic DNA in conditions at least as stringent as 4×SSC at 50 degrees C.;
   (iii) amplifying human DNA sequences; and
   (iv) isolating the polynucleotide products of step (b)(iii).
      Preferably the polynucleotide isolated according to the above process comprises a nucleotide sequence corresponding to the cDNA sequence of SEQ ID NO:215, and extending contiguously from a nucleotide sequence corresponding to the 5′ end of SEQ ID NO:215 to a nucleotide sequence corresponding to the 3′ end of SEQ ID NO:215, but excluding the poly(A) tail at the 3′ end of SEQ ID NO:215. Also preferably the polynucleotide isolated according to the above process comprises a nucleotide sequence corresponding to the cDNA sequence of SEQ ID NO:215 from nucleotide 30 to nucleotide 434, and extending contiguously from a nucleotide sequence corresponding to the 5′ end of said sequence of SEQ ID NO:215 from nucleotide 30 to nucleotide 434, to a nucleotide sequence corresponding to the 3′ end of said sequence of SEQ ID NO:215 from nucleotide 30 to nucleotide 434. Also preferably the polynucleotide isolated according to the above process comprises a nucleotide sequence corresponding to the cDNA sequence of SEQ ID NO:215 from nucleotide 149 to nucleotide 447, and extending contiguously from a nucleotide sequence corresponding to the 5′ end of said sequence of SEQ ID NO:215 from nucleotide 149 to nucleotide 447, to a nucleotide sequence corresponding to the 3′ end of said sequence of SEQ ID NO:215 from nucleotide 149 to nucleotide 447.

In other embodiments, the present invention provides a composition comprising a protein, wherein said protein comprises an amino acid sequence selected from the group consisting of:
(a) the amino acid sequence of SEQ ID NO:216;
(b) the amino acid sequence of SEQ ID NO:216 from amino acid 41 to amino acid 135;
(c) a fragment of the amino acid sequence of SEQ ID NO:216, the fragment comprising eight contiguous amino acids of SEQ ID NO:216; and
(d) the amino acid sequence encoded by the cDNA insert of clone CC288_{—}9 deposited with the ATCC under accession number 98146;
   the protein being substantially free from other mammalian proteins. Preferably such protein comprises the amino acid sequence of SEQ ID NO:216 or the amino acid sequence of SEQ ID NO:216 from amino acid 41 to amino acid 135. In further preferred embodiments, the present invention provides a protein comprising a fragment of the amino acid sequence of SEQ ID NO:216 having biological activity, the fragment preferably comprising eight (more preferably twenty, most preferably thirty) contiguous amino acids of SEQ ID NO:216, or a protein comprising a fragment of the amino acid sequence of SEQ ID NO:216 having biological activity, the fragment comprising the amino acid sequence from amino acid 62 to amino acid 71 of SEQ ID NO:216.

In certain preferred embodiments, the polynucleotide is operably linked to an expression control sequence. The invention also provides a host cell, including bacterial, yeast, insect and mammalian cells, transformed with such polynucleotide compositions. Also provided by the present invention are organisms that have enhanced, reduced, or modified expression of the gene(s) corresponding to the polynucleotide sequences disclosed herein.

Processes are also provided for producing a protein, which comprise:
(a) growing a culture of the host cell transformed with such polynucleotide compositions in a suitable culture medium; and
(b) purifying the protein from the culture.
   The protein produced according to such methods is also provided by the present invention.

Protein compositions of the present invention may further comprise a pharmaceutically acceptable carrier. Compositions comprising an antibody which specifically reacts with such protein are also provided by the present invention.

Methods are also provided for preventing, treating or ameliorating a medical condition which comprises administering to a mammalian subject a therapeutically effective amount of a composition comprising a protein of the present invention and a pharmaceutically acceptable carrier.

### BRIEF DESCRIPTION OF THE DRAWINGS

FIGS. 1A and 1B are schematic representations of the pED6 and pNOTs vectors, respectively, used for deposit of clones disclosed herein.

FIG. 2 is an autoradiograph showing the products of expression of clone B18_{—}11 in COS cells (with the protein band indicated by an arrow).

FIG. 3 is an autoradiograph showing the products of expression of clone H174_{—}10 in COS cells (with the protein band indicated by an arrow).

FIG. 4 is an autoradiograph showing the products of expression of clone J5_{—}3 in COS cells (with the protein bands indicated by arrows). J5_{—}3 produces multiple protein bands, with the major band at approximately 58 kD.

FIG. 5 is an autoradiograph showing the products of expression of clone L105_{—}74 in COS cells (with the protein bands indicated by arrows). L105_{—}74 produces multiple protein bands, with the major band at approximately 15 kD.

FIG. 6 is an autoradiograph showing the products of expression of clones B121_{—}1 and B18_{—}11 in COS cells (with the protein bands indicated by dots).

FIG. 7 is an autoradiograph showing the products of expression of clone B121_{—}1 in baculovirus (with the protein bands indicated by dots).

FIG. 8 is an autoradiograph showing the products of expression of clone B219_{—}2 in COS cells.

FIG. 9 is an autoradiograph showing the products of expression of clone G52_{—}24 in COS cells.

FIG. 10 is an autoradiograph showing the products of expression of the following clones in COS cells: H83_{—}22, D147_{—}17, H298_{—}23, and H849_{—}24 (with the protein bands indicated by dots).

FIG. 11 is an autoradiograph showing the products of expression of clone AZ302_{—}1 in COS cells (with the protein band(s) indicated by dot(s)).

FIG. 12 is an autoradiograph showing the products of expression of clone BG33_{—}7 in COS cells (with the protein band indicated by a dot).

FIG. 13 is an autoradiograph showing the products of expression of clone H438_{—}1 in COS cells (with the protein band indicated by a dot).

### DETAILED DESCRIPTION

Isolated Proteins and Polynucleotides

Nucleotide and amino acid sequences, as presently determined, are reported below for each clone and protein disclosed in the present application. The nucleotide sequence of each clone can readily be determined by sequencing of the deposited clone in accordance with known methods. The predicted amino acid sequence (both full-length and mature forms) can then be determined from such nucleotide sequence. The amino acid sequence of the protein encoded by a particular clone can also be determined by expression of the clone in a suitable host cell, collecting the protein and determining its sequence. For each disclosed protein applicants have identified what they have determined to be the reading frame best identifiable with sequence information available at the time of filing.

As used herein a “secreted” protein is one which, when expressed in a suitable host cell, is transported across or through a membrane, including transport as a result of signal sequences in its amino acid sequence. “Secreted” proteins include without limitation proteins secreted wholly (e.g., soluble proteins) or partially (e.g., receptors) from the cell in which they are expressed. “Secreted” proteins also include without limitation proteins which are transported across the membrane of the endoplasmic reticulum.

Clone “B18_{—}11”

A polynucleotide of the present invention has been identified as clone “B18_{—}11”. B18_{—}11 was isolated from a human adult blood (peripheral blood mononuclear cells activated by treatment with concanavalin A and phorbol myristate acetate) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. B18_{—}11 is a full-length clone, including the entire coding sequence of a secreted protein (also referred to herein as “B18_{—}11 protein”). B18_{—}11 (human CTLA-8) polynucleotides and proteins are also described in WO 97/04097.

The nucleotide sequence of B18_{—}11 as presently determined is reported in SEQ ID NO:1, and includes a poly(A) tail. What applicants presently believe to be the proper reading frame and the predicted amino acid sequence of the B18_{—}11 protein corresponding to the foregoing nucleotide sequence is reported in SEQ ID NO:2. Amino acids 18 to 30 of SEQ ID NO:2 are a predicted leader/signal sequence, with the predicted mature amino acid sequence beginning at amino acid 31.

The EcoRI/NotI restriction fragment obtainable from the deposit containing clone B18_{—}11 should be approximately 400 bp.

The nucleotide sequence disclosed herein for B18_{—}11 was searched against the GenBank and GeneSeq nucleotide sequence databases using BLASTN/BLASTX and FASTA search protocols. B18_{—}11 demonstrated at least some similarity with sequences identified as M60286 (Herpesvirus saimiri immediate early region protein genes, complete cds), Q92884 (Human CTLA-8 cDNA), T61413 (Human CTLA-8), and U32659 (Human IL-17 mRNA, complete cds). The predicted amino acid sequence disclosed herein for B18_{—}11 was searched against the GenPept and GeneSeq amino acid sequence databases using the BLASTX search protocol. The predicted B18_{—}11 protein demonstrated at least some similarity to sequences identified as L13839 (Mus musculus or Rattus rattus (clone 2.6) CTLA-8 mRNA sequence, complete cds), M60286 (immediate-early protein [Herpesvirus saimiri]), R76573 (Human CTLA-8), R76574 (Human CTLA-8 mature protein), U32659 (IL-17 [Homo sapiens]), W13651 (Human CTLA-8), and X64346 (ORF 13; KCLF2 [Saimiriine herpesvirus 2]). The region from amino acid 29 to amino acid 163 of B18_{—}11 protein (SEQ ID NO:2) shows marked amino acid similarity to portions of murine CTLA-8 (amino acids 18 to 150, GenBank accession L13839) and herpesvirus Saimiri ORF13 (“herpes CTLA-8”) (amino acids 19 to 151, GenBank accession X64346). Based upon these similarities, B18_{—}11 is believed to be the human homolog of murine and herpes CTLA-8 (i.e., “human CTLA-8”).

B18_{—}11 protein was expressed in a COS cell expression system, and an expressed protein band of approximately 20 kDa was detected in conditioned medium using SDS polyacrylamide gel electrophoresis.

Isolates “H174_{—}10” and “H174_{—}43” of Clone H174

Isolated polynucleotides of the present invention have been identified as clone “H174_{—}10” and as clone “H174_{—}43” (collectively referred to herein as clone “H174”). H174_{—}10 and H174_{—}43 were isolated from a human adult peripheral blood mononuclear cell (activated by treatment with phytohemagglutinin, phorbol myristate acetate, and mixed lymphocyte reaction) cDNA library, and were identified as encoding a secreted or transmembrane protein using methods which are selective for cDNAs encoding secreted proteins (see U.S. Pat. No. 5,536,637). H174_{—}10 and H174_{—}43 are full-length clones, each including the entire coding sequence of a secreted protein (also referred to herein as “H174 protein”).

The nucleotide sequences of H174_{—}10 and H174_{—}43 as presently determined are reported in SEQ ID NO:3 and in SEQ ID NO:5, respectively, and in the case of H174_{—}10 includes a poly(A) tail. What applicants presently believe to be the proper reading frame and the predicted amino acid sequence of the H174_{—}10 and H174_{—}43 proteins corresponding to the foregoing nucleotide sequences are reported in SEQ ID NO:4 and SEQ ID NO:6, respectively. Amino acids 9 to 21 of both SEQ ID NO:4 and SEQ ID NO:6 are predicted leader/signal sequences, with the predicted mature amino acid sequence beginning in each case at amino acid 22.

The EcoRI/NotI restriction fragment obtainable from the deposit containing clone H174_{—}10 should be approximately 967 bp, and the EcoRI/NotI restriction fragment corresponding to clone H174_{—}43 should be approximately 1354 bp.

The nucleotide sequence disclosed herein for H174_{—}10 was searched against the GenBank and GeneSeq nucleotide sequence databases using BLASTN/BLASTX and FASTA search protocols. H174_{—}10 demonstrated at least some similarity with sequences identified as U59286 (Human beta-R1 mRNA, partial cds). The predicted amino acid sequence disclosed herein for H174_{—}10 was searched against the GenPept and GeneSeq amino acid sequence databases using the BLASTX search protocol. The predicted H174_{—}10 protein demonstrated at least some similarity to sequences identified as AF00298 (putative alpha chemokine [Homo sapiens]), M86829 (homolog of human IP-10 [Mus musculus]), R25341 (MIG-2), R70791 (Gamma-IP-10), and U59286 (Orf1 [Homo sapiens]). Based upon sequence similarity, H174_{—}10 proteins and each similar protein or peptide may share at least some activity. Beta-R1 is selectively expressed in response to interferon beta (IFN-beta) compared with IFN-alpha, and H174_{—}10 protein also shows similarity to several other interferon-induced proteins. The TopPredII computer program detects a potential transmembrane domain within the H174_{—}10 protein sequence, centered around amino acid 22 of SEQ ID NO:4.

H174_{—}10 protein was expressed in a COS cell expression system, and an expressed protein band of approximately 7 kDa was detected in conditioned medium and membrane fractions using SDS polyacrylamide gel electrophoresis.

Clone “J5_{—}3”

A polynucleotide of the present invention has been identified as clone “J5_{—}3”. J5_{—}3 was isolated from a human adult blood (peripheral blood mononuclear cells activated by treatment with phytohemagglutinin, phorbol myristate acetate, and mixed lymphocyte reaction) cDNA library using methods which are selective for cDNAs encoding secreted proteins (see U.S. Pat. No. 5,536,637). J5_{—}3 is a full-length clone, including the entire coding sequence of a secreted protein (also referred to herein as “J5_{—}3 protein”).

The nucleotide sequence of J5_{—}3 as presently determined is reported in SEQ ID NO:7. What applicants presently believe to be the proper reading frame and the predicted amino acid sequence of the J5_{—}3 protein corresponding to the foregoing nucleotide sequence is reported in SEQ ID NO:8. Amino acids 16 to 28 of SEQ ID NO:8 are a predicted leader/signal sequence, with the predicted mature amino acid sequence beginning at amino acid 29.

The EcoRI/NotI restriction fragment obtainable from the deposit containing clone J5_{—}3 should be approximately 2209 bp.

The nucleotide sequence disclosed herein for J5_{—}3 was searched against the GenBank and GeneSeq nucleotide sequence databases using BLASTN/BLASTX and FASTA search protocols. J5_{—}3 demonstrated at least some similarity with sequences identified as N90239 (Clone SVG48 (FIB 48) encoding southern copperhead fibrolase), T63123 (Human metalloproteinase DNA), X66139 (M. fascicularis mRNA for epididymal apical protein I), Y13323 (Homo sapiens mRNA for disintegrin-protease), and Z81161 (S. scrofa mRNA; expressed sequence tag (5′; clone c5c05)). The predicted amino acid sequence disclosed herein for J5_{—}3 was searched against the GenPept and GeneSeq amino acid sequence databases using the BLASTX search protocol. The predicted J5_{—}3 protein demonstrated at least some similarity to sequences identified as P90457 (Snake venom fibrolase from SouthernCopperhead), U01235 (prepro-hemorrhagic toxin b [Crotalus atrox]), U86634 (Agkistrodon contortrix laticinctus metalloproteinase-disintegrin-like protein mRNA, complete cds), W14772 (Human metalloproteinase), X66139 (epididymal apical protein I-precursor [Macaca fascicularis]), X68251 (jararhagin [Bothrops jararaca]), and Y13323 (disintegrin-protease [Homo sapiens]). Based upon sequence similarity, J5_{—}3 proteins and each similar protein or peptide may share at least some activity. Analysis of the full-length J5_{—}3 amino acid sequence revealed that the disintegrin domain (amino acids 423 to 463 of SEQ ID NO:8) was incomplete and that this clone did not contain an EGF-domain, as with some of the other disintegrin family members; however, J5_{—}3 protein does contain a conserved “prodomain” (amino acids 1 to 217 of SEQ ID NO:8) and a metalloproteinase domain (amino acids 218 to 422 of SEQ ID NO:8) that contains a zinc-binding motif (amino acids 352 to 359 of SEQ ID NO:8). Disintegrin domains have been associated with particular biological activities, particularly inhibition of platelet aggregation (see Usami et al., 1994, Biochem. Biophys. Res. Comm. 201(1): 331-339; Paine et al., 1992, J. Biol. Chem. 267(32): 22869-22876; Shigeta et al., 1992, Circulation 86(5 Suppl): II 398-404; and Takeya et al., 1992, J. Biol. Chem. 267(20): 14109-14117), inhibition of metastatic cancer cell adhesion (see Trikha et al., 1994, Cancer Res. 54(18): 4993-4998), and promotion of egg-sperm cell fusion (see Myles et al., 1994, Proc. Natl. Acad. Sci. USA 91(10): 4195-4198; and Wolfsberg et al., 1993, Proc. Natl. Acad. Sci. USA 90(22): 10783-10787). Metalloproteinases have also been shown to be involved in cancer metastasis and tumor angiogenesis (see Kohn et al., 1995, Cancer Res. 55(9): 1856-1862). The TopPredII computer program detects a potential transmembrane domain within the J5_{—}3 protein sequence centered around amino acid 29 of SEQ ID NO:8.

J5_{—}3 protein was expressed in a COS cell expression system, and an expressed protein band of approximately 60 kDa was detected in conditioned medium using SDS polyacrylamide gel electrophoresis.

Clone “J422_{—}1”

A polynucleotide of the present invention has been identified as clone “J422_{—}1”. J422_{—}21 was isolated from a human adult blood (peripheral blood mononuclear cells activated by treatment with phytohemagglutinin, phorbol myristate acetate, and mixed lymphocyte reaction) cDNA library using methods which are selective for cDNAs encoding secreted proteins (see U.S. Pat. No. 5,536,637). J422_{—}1 is a full-length clone, including the entire coding sequence of a secreted protein (also referred to herein as “J422_{—}1 protein”).

The nucleotide sequence of J422_{—}1 as presently determined is reported in SEQ ID NO:9, and includes a poly(A) tail. What applicants presently believe to be the proper reading frame and the predicted amino acid sequence of the J422_{—}1 protein corresponding to the foregoing nucleotide sequence is reported in SEQ ID NO:10. Amino acids 501 to 513 of SEQ ID NO:10 are a predicted leader/signal sequence, with the predicted mature amino acid sequence beginning at amino acid 514. Due to the hydrophobic nature of the predicted leader/signal sequence, it is likely to act as a transmembrane domain should the predicted leader/signal sequence not be separated from the remainder of the J422_{—}1 protein.

The EcoRI/NotI restriction fragment obtainable from the deposit containing clone J422_{—}1 should be approximately 2582 bp.

The nucleotide sequence disclosed herein for J422_{—}1 was searched against the GenBank and GeneSeq nucleotide sequence databases using BLASTN/BLASTX and FASTA search protocols. J422_{—}1 demonstrated at least some similarity with sequences identified as AA252850 (zs27f02.r1 Soares NbHTGBC Homo sapiens cDNA clone 686427 5′), AA291242 (zs47e10.r1 NCI_CGAP_GCB1 Homo sapiens cDNA clone 700650 5′ similar to TR:G761712 G761712 RP105 PRECURSOR), D37797 (Mouse mRNA for RP105), D83597 (Human mRNA for RP105, complete cds), and T72271 (Human B cell surface antigen cDNA). The predicted amino acid sequence disclosed herein for J422_{—}1 was searched against the GenPept and GeneSeq amino acid sequence databases using the BLASTX search protocol. The predicted J422_{—}1 protein demonstrated at least some similarity to sequences identified as D37797 (RP105 [Mus musculus]), D83597 (RP105 [Homo sapiens]), R85888 (WD-40 domain-containing insulin-like growth factor binding protein), and X53959 (slit protein [Drosophila melanogaster]), and a number of Drosophila leucine-rich repeat (LRR) proteins. RP105 is a B cell surface molecule that transmits a growth-promoting signal and is implicated in the life/death decision of B cells. RP105 has tandem repeats of a leucine-rich motif in the extracellular domain that is expected to be involved in protein-protein interactions. Analysis of the full-length J422_{—}1 amino acid sequence revealed that the conserved EGF-domain found in a number of LRR family members was not present in J422_{—}1. Based upon sequence similarity, J422_{—}1 proteins and each similar protein or peptide may share at least some activity. The TopPredII computer program predicts a potential transmembrane domain within the J422_{—}1 protein sequence centered around amino acid 630 of SEQ ID NO:10.

Clone “L105_{—}74”

A polynucleotide of the present invention has been identified as clone “L105_{—}74”. L105_{—}74 was isolated from a murine adult thymus cDNA library using methods which are selective for cDNAs encoding secreted proteins (see U.S. Pat. No. 5,536,637). L105_{—}74 is a full-length clone, including the entire coding sequence of a secreted protein (also referred to herein as “L105_{—}74 protein”). Human cDNA clones (L105_{—}3 or L261_{—}1, and L105_{—}7 or L262_{—}1) corresponding to the murine L105_{—}74 have also been identified (see WO 98/56818).

The nucleotide sequence of L105_{—}74 as presently determined is reported in SEQ ID NO:11. What applicants presently believe to be the proper reading frame and the predicted amino acid sequence of the L105_{—}74 protein corresponding to the foregoing nucleotide sequence is reported in SEQ ID NO:12. Amino acids 3 to 15 of SEQ ID NO:12 are a predicted leader/signal sequence, with the predicted mature amino acid sequence beginning at amino acid 16. Due to the hydrophobic nature of the predicted leader/signal sequence, it is likely to act as a transmembrane domain should the predicted leader/signal sequence not be separated from the remainder of the L105_{—}74 protein.

The EcoRI/NotI restriction fragment obtainable from the deposit containing clone L105_{—}74 should be approximately 615 bp.

The nucleotide sequence disclosed herein for L105_{—}74 was searched against the GenBank and GeneSeq nucleotide sequence databases using BLASTN/BLASTX and FASTA search protocols. L105_{—}74 demonstrated at least some similarity with sequences identified as AA220126 (mx99f02.r1 Barstead mouse lung MPLRB2 Mus musculus cDNA clone 694491 5′), AF006637 (Mus musculus beta-chemokine TCA4 mRNA, complete cds), and T18015 (Chemokine beta-9 coding sequence). The predicted amino acid sequence disclosed herein for L105_{—}74 was searched against the GenPept and GeneSeq amino acid sequence databases using the BLASTX search protocol. The predicted L105_{—}74 protein demonstrated at least some similarity to sequences identified as AF00198 (beta chemokine [Mus musculus]), AF006637 (TCA4 [Mus musculus]), L34553 (cytokine [Gallus gallus]), R81567 (Chemokine beta-9), and U77180 (macrophage inflammatory protein 3 beta [Homo sapiens]). Based upon sequence similarity, L105_{—}74 proteins and each similar protein or peptide may share at least some activity. The TopPredII computer program predicts a potential transmembrane domain within the amino terminal portion of the L105_{—}74 protein sequence.

L105_{—}74 protein was expressed in a COS cell expression system, and an expressed protein band of approximately 17 kDa was detected in conditioned medium and membrane fractions using SDS polyacrylamide gel electrophoresis.

Clone “B121_{—}1”

A polynucleotide of the present invention has been identified as clone “B121_{—}1”. A cDNA clone was first isolated from a human adult blood (peripheral blood mononuclear cells activated by treatment with concanavalin A and phorbol myristate acetate) cDNA library using methods which are selective for cDNAs encoding secreted proteins (see U.S. Pat. No. 5,536,637). This cDNA clone was then used to isolate B121_{—}1 from a human adult blood (peripheral blood mononuclear cells activated by treatment with phytohemagglutinin, phorbol myristate acetate, and mixed lymphocyte reaction) cDNA library. B121_{—}1 is a full-length clone, including the entire coding sequence of a secreted protein (also referred to herein as “B121_{—}1 protein”).

The nucleotide sequence of B121_{—}1 as presently determined is reported in SEQ ID NO:13, and includes a poly(A) tail. What applicants presently believe to be the proper reading frame and the predicted amino acid sequence of the B121_{—}1 protein corresponding to the foregoing nucleotide sequence is reported in SEQ ID NO:14. Amino acids 98 to 110 of SEQ ID NO:14 are a predicted leader/signal sequence, with the predicted mature amino acid sequence beginning at amino acid 111. Amino acids 23 to 35 of SEQ ID NO:14 are a possible leader/signal sequence, with the predicted mature amino acid sequence beginning at amino acid 36. Due to the hydrophobic nature of these predicted leader/signal sequences, each is likely to act as a transmembrane domain should it not be separated from the remainder of the B121_{—}1 protein.

The EcoRI/NotI restriction fragment obtainable from the deposit containing clone B121_{—}1 should be approximately 1760 bp.

The nucleotide sequence disclosed herein for B121_{—}1 was searched against the GenBank and GeneSeq nucleotide sequence databases using BLASTN/BLASTX and FASTA search protocols. B121_{—}1 demonstrated at least some similarity with sequences identified as AA136867 (zl01c02.s1 Soares pregnant uterus NbHPU Homo sapiens cDNA clone 491042 3′), H23221 (ym52f07.s1 Homo sapiens cDNA clone 51884 3′), R83586 (yp16a07.r1 Homo sapiens cDNA clone 187572 5′), and W72694 (zd68f10.s1 Soares fetal heart NbHH19W Homo sapiens cDNA clone 345835 3′ similar to contains Alu repetitive element). The predicted amino acid sequence disclosed herein for B121_{—}1 was searched against the GenPept and GeneSeq amino acid sequence databases using the BLASTX search protocol. The predicted B121_{—}1 protein demonstrated at least some similarity with sequences identified as U28928 (C44B7.4 gene product [Caenorhabditis elegans]). Based upon sequence similarity, B121_{—}1 proteins and each identical protein or peptide may share at least some activity. The TopPredII computer program predicts two additional potential trans-membrane domains within the B121_{—}1 protein sequence, one centered around amino acid 60 and another around amino acid 190 of SEQ ID NO:14. The nucleotide sequence of B121_{—}1 indicates that it may contain a Mer repetitive element.

B121_{—}1 protein was expressed both in baculovirus and in a COS cell expression system, and an expressed protein band of approximately 90 kDa was detected in conditioned medium using SDS polyacrylamide gel electrophoresis.

Clone “B196_{—}122”

A polynucleotide of the present invention has been identified as clone “B196_{—}122”. A cDNA clone was first isolated from a human adult blood (peripheral blood mononuclear cells activated by treatment with concanavalin A and phorbol myristate acetate) cDNA library using methods which are selective for cDNAs encoding secreted proteins (see U.S. Pat. No. 5,536,637). This cDNA clone was then used to isolate B196_{—}122 from a human adult blood (peripheral blood mononuclear cells activated by treatment with phytohemagglutinin, phorbol myristate acetate, and mixed lymphocyte reaction) cDNA library. B196_{—}122 is a full-length clone, including the entire coding sequence of a secreted protein (also referred to herein as “B196_{—}122 protein”).

The nucleotide sequence of B196_{—}122 as presently determined is reported in SEQ ID NO:15, and includes a poly(A) tail. What applicants presently believe to be the proper reading frame and the predicted amino acid sequence of the B196_{—}122 protein corresponding to the foregoing nucleotide sequence is reported in SEQ ID NO:16. Amino acids 92 to 104 of SEQ ID NO:16 are a predicted leader/signal sequence, with the predicted mature amino acid sequence beginning at amino acid 105. Due to the hydrophobic nature of the predicted leader/signal sequence, it is likely to act as a transmembrane domain should the predicted leader/signal sequence not be separated from the remainder of the B196_{—}122 protein. Additional nucleotide sequence for B196 is reported in SEQ ID NO:320. Applicants believe SEQ ID NO:320 represents a cDNA molecule produced from an immature mRNA transcript, as base pairs 205 to 352 of SEQ ID NO:320 appear to be an intron sequence. SEQ ID NO:15 was derived from SEQ ID NO:320 by deleting this presumed intron sequence.

The EcoRI/NotI restriction fragment obtainable from the deposit containing clone B196_{—}122 should be approximately 1800 bp.

The nucleotide sequence disclosed herein for B196_{—}122 was searched against the GenBank and GeneSeq nucleotide sequence databases using BLASTN/BLASTX and FASTA search protocols. B196_{—}122 demonstrated at least some similarity with sequences identified as AA235452 (zt35c01.s1 Soares ovary tumor NbHOT Homo sapiens cDNA clone 724320 3′ similar to contains Alu repetitive element), T09157 (EST07050 Homo sapiens cDNA clone HIBBP87 5′ end), T34456 (EST68380 Homo sapiens cDNA 5′ end similar to None), T35039 (EST79238 Homo sapiens cDNA similar to None), and T70971 (yc49f08.r1 Homo sapiens cDNA clone 84039 5′). Based upon sequence similarity, B196_{—}122 proteins and each similar protein or peptide may share at least some activity.

Clone “D157_{—}4”

A polynucleotide of the present invention has been identified as clone “D157_{—}4”. A cDNA clone was first isolated from a human adult blood (peripheral blood mononuclear cells activated by treatment with concanavalin A and phorbol myristate acetate) cDNA library using methods which are selective for cDNAs encoding secreted proteins (see U.S. Pat. No. 5,536,637). This cDNA clone was then used to isolate D157_{—}4 from a human adult blood (peripheral blood mononuclear cells activated by treatment with phytohemagglutinin, phorbol myristate acetate, and mixed lymphocyte reaction) cDNA library. D157_{—}4 is a full-length clone, including the entire coding sequence of a secreted protein (also referred to herein as “D157_{—}4 protein”).

The nucleotide sequence of D157_{—}4 as presently determined is reported in SEQ ID NO:17, and includes a poly(A) tail. What applicants presently believe to be the proper reading frame and the predicted amino acid sequence of the D157_{—}4 protein corresponding to the foregoing nucleotide sequence is reported in SEQ ID NO:18. Amino acids 1 to 9 of SEQ ID NO:18 are a predicted leader/signal sequence, with the predicted mature amino acid sequence beginning at amino acid 10. Amino acids 68 to 80 of SEQ ID NO:18 are also a predicted leader/signal sequence, with the predicted mature amino acid sequence beginning in that case at amino acid 81. Due to the hydrophobic nature of this additional predicted leader/signal sequence, it is likely to act as a transmembrane domain should it not be separated from the remainder of the D157_{—}4 protein.

The EcoRI/NotI restriction fragment obtainable from the deposit containing clone D157_{—}4 should be approximately 639 bp.

The nucleotide sequence disclosed herein for D157_{—}4 was searched against the GenBank and GeneSeq nucleotide sequence databases using BLASTN/BLASTX and FASTA search protocols. D157_{—}4 demonstrated at least some similarity with sequences identified as AA112439 (zm27f08.r1 Stratagene pancreas (#937208) Homo sapiens cDNA clone 526887 5′ similar to TR G1277178 G1277178 YMP), AA156309 (zo54a07.s1 Stratagene pancreas (#937208) Homo sapiens cDNA clone 590676 3′ similar to TR G1277178 G1277178 YMP), H45474 (yo72g03.s1 Homo sapiens cDNA clone 183508 3′ similar to gb D11428 PERIPHERAL MYELIN PROTEIN 22 (HUMAN)), H45571 (yo72g04.r1 Soares breast 3NbHBst Homo sapiens cDNA clone IMAGE:183510 5′ similar to SP:PM22_RAT P25094 PERIPHERAL MYELIN PROTEIN 22; mRNA sequence), Q32869 (Human PMP-22 cDNA), U52101 (Human YMP mRNA, complete cds), U87947 (Human hematopoietic neural membrane protein (HNMP-1) mRNA, complete cds), W73810 (zd52c09.s1 Soares fetal heart NbHH19W Homo sapiens cDNA clone 344272 3′ similar to SW PM22_HUMAN Q01453 PERIPHERAL MYELIN PROTEIN 22; contains element TAR1 repetitive element), and X94771 (H. sapiens mRNA for epithelial membrane protein-3). The predicted amino acid sequence disclosed herein for D157_{—}4 was searched against the GenPept and GeneSeq amino acid sequence databases using the BLASTX search protocol. The predicted D157_{—}4 protein demonstrated at least some similarity to sequences identified as M94048 (peripheral myelin protein 22 [Homo sapiens]), R30056 (Human PMP-22), U52101 (YMP [Homo sapiens]), and U87947 (hematopoietic neural membrane protein [Homo sapiens]). Based upon sequence similarity, D157_{—}4 proteins and each similar protein or peptide may share at least some activity. The TopPredII computer program predicts three potential transmembrane domains within the D157_{—}4 protein sequence, centered around amino acids 80, 110, and 150 of SEQ ID NO:18, respectively.

D157_{—}4 protein was expressed in a COS cell expression system, and an expressed protein band of approximately 15 kDa was detected in conditioned medium and membrane fractions using SDS polyacrylamide gel electrophoresis.

Clone “B219_{—}2”

A polynucleotide of the present invention has been identified as clone “B219_{—}2”. A cDNA clone was first isolated from a human adult blood (peripheral blood mononuclear cells activated by treatment with concanavalin A and phorbol myristate acetate) cDNA library using methods which are selective for cDNAs encoding secreted proteins (see U.S. Pat. No. 5,536,637). This cDNA clone was then used to isolate B219_{—}2 from a human adult blood (peripheral blood mononuclear cells activated by treatment with phytohemagglutinin, phorbol myristate acetate, and mixed lymphocyte reaction) cDNA library. B219_{—}2 is a full-length clone, including the entire coding sequence of a secreted protein (also referred to herein as “B219_{—}2 protein”).

The nucleotide sequence of B219_{—}2 as presently determined is reported in SEQ ID NO:19, and includes a poly(A) tail. What applicants presently believe to be the proper reading frame and the predicted amino acid sequence of the B219_{—}2 protein corresponding to the foregoing nucleotide sequence is reported in SEQ ID NO:20.

The EcoRI/NotI restriction fragment obtainable from the deposit containing clone B219_{—}2 should be approximately 850 bp.

The nucleotide sequence disclosed herein for B219_{—}2 was searched against the GenBank and GeneSeq nucleotide sequence databases using BLASTN/BLASTX and FASTA search protocols. B219_{—}2 demonstrated at least some similarity with sequences identified as AA188024 (zp68h11.s1 Stratagene endothelial cell 937223 Homo sapiens cDNA clone 625413 3′ similar to SW NIAM_BOVIN Q02372 NADH-UBIQUINONE OXIDOREDUCTASE ASHI SUBUNIT PRECURSOR), N51696 (yy72c05.s1 Homo sapiens cDNA clone 279080 3′ similar to SW:NIAM_BOVIN Q02372 NADH-UBIQUINONE OXIDOREDUCTASE ASHI SUBUNIT PRECURSOR), R11886 (yf49g09.r1 Soares infant brain 1NIB Homo sapiens cDNA clone IMAGE:25515 5′ similar to SP:NIAM_BOVIN Q02372 NADH-UBIQUINONE OXIDOREDUCTASE ASHI SUBUNIT PRECURSOR; mRNA sequence), R53932 (yg83a06.s1 Homo sapiens cDNA clone 39817 3′ similar to SP:NIAM_BOVIN Q02372 NADH-UBIQUINONE OXIDOREDUCTASE ASHI SUBUNIT PRECURSOR), T20735 (Human gene signature HUMGS01953), T30635 (EST19946 Homo sapiens cDNA 5′ end similar to similar to NADH-ubiquinone oxidoreductase, ASHI subunit), and X63209 (B. taurus CI-ASHI mRNA for ubiquinone oxidoreductase complex). The predicted amino acid sequence disclosed herein for B219_{—}2 was searched against the GenPept and GeneSeq amino acid sequence databases using the BLASTX search protocol. The predicted B219_{—}2 protein demonstrated at least some similarity to sequences identified as R34767 (N-terminus of NADH/ubiquinone oxidoreductase, termed ASHI) and X63209 (NADH dehydrogenase [Bos taurus]). The function of ubiquinone oxidoreductase is the transfer of electrons from NADH to the respiratory chain. The subcellular location of ubiquinone oxidoreductase is the mitochondrial inner membrane, matrix side. The immediate electron acceptor for the enzyme is believed to be ubiquinone, with the following catalytic activity: NADH+ubiquinone->NAD(+)+ubiquinol. Based upon sequence similarity, B219_{—}2 proteins and each similar protein or peptide may share at least some activity. The TopPredII computer program predicts a potential transmembrane domain within the B219_{—}2 protein sequence centered around amino acid 130 of SEQ ID NO:20.

B219_{—}2 protein was expressed in a COS cell expression system, and an expressed protein band of approximately 21 kDa was detected in membrane fractions using SDS polyacrylamide gel electrophoresis.

Clone “G52_{—}24”

A polynucleotide of the present invention has been identified as clone “G52_{—}24”. A cDNA clone was first isolated from a human adult blood (peripheral blood mononuclear cells activated by treatment with concanavalin A and phorbol myristate acetate) cDNA library using methods which are selective for cDNAs encoding secreted proteins (see U.S. Pat. No. 5,536,637). This cDNA clone was then used to isolate G52_{—}24 from a human adult blood (peripheral blood mononuclear cells activated by treatment with phytohemagglutinin, phorbol myristate acetate, and mixed lymphocyte reaction) cDNA library. G52_{—}24 is a full-length clone, including the entire coding sequence of a secreted protein (also referred to herein as “G52_{—}24 protein”).

The nucleotide sequence of G52_{—}24 as presently determined is reported in SEQ ID NO:21, and includes a poly(A) tail. What applicants presently believe to be the proper reading frame and the predicted amino acid sequence of the G52_{—}24 protein corresponding to the foregoing nucleotide sequence is reported in SEQ ID NO:22. Amino acids 28 to 40 of SEQ ID NO:22 are a predicted leader/signal sequence, with the predicted mature amino acid sequence beginning at amino acid 41. Due to the hydrophobic nature of the predicted leader/signal sequence, it is likely to act as a transmembrane domain should the predicted leader/signal sequence not be separated from the remainder of the G52_{—}24 protein. G52_{—}24 appears to be a splice variant of clone H298_{—}23 described below.

The EcoRI/NotI restriction fragment obtainable from the deposit containing clone G52_{—}24 should be approximately 1588 bp.

The nucleotide sequence disclosed herein for G52_{—}24 was searched against the GenBank and GeneSeq nucleotide sequence databases using BLASTN/BLASTX and FASTA search protocols. G52_{—}24 demonstrated at least some similarity with sequences identified as AA029932 (zk08e11.r1 Soares pregnant uterus NbHPU Homo sapiens cDNA clone 469964 5′ similar to SW CD69_HUMAN Q07108 EARLY ACTIVATION ANTIGEN CD690, AA028937 (zk08e11.s1 Soares pregnant uterus NbHPU Homo sapiens cDNA clone 469964 3′), X73752 (CHESTM049 Goat mammary gland Capra hircus cDNA, mRNA sequence), X87344 (H. sapiens DMA, DMB, HLA-Z1, IPP2, LMP2, TAP1, LMP7, TAP2, DOB, DQB2 and RING8, 9, 13 and 14 genes), and X96719 (H. sapiens mRNA for AICL (activation-induced C-type lectin)). The predicted amino acid sequence disclosed herein for G52_{—}24 was searched against the GenPept and GeneSeq amino acid sequence databases using the BLASTX search protocol. The predicted G52_{—}24 protein demonstrated at least some similarity to sequences identified as L07555 (early activation antigen CD69 [Homo sapiens]), R54659 (Human CD69), and X96719 (C-Type lectin [Homo sapiens]). G52_{—}24 protein appears to contain a C-type lectin domain. Lectin domains are involved in the calcium-dependent binding of certain carbohydrate moeities such as sialyl-Lewis-X, and the glycoproteins that bear them. Based upon sequence similarity, G52_{—}24 proteins and each similar protein or peptide may share at least some activity. The TopPredII computer program predicts a potential transmembrane domain within the G52_{—}24 protein sequence centered around amino acid 40 of SEQ ID NO:22. The nucleotide sequence of G52_{—}24 indicates that it may contain one or more repetitive element sequences.

G52_{—}24 protein was expressed in a COS cell expression system, and an expressed protein band of approximately 28 kDa was detected in conditioned medium using SDS polyacrylamide gel electrophoresis.

Clone “G86_{—}2”

A polynucleotide of the present invention has been identified as clone “G86_{—}2”. A cDNA clone was first isolated from a human adult blood (peripheral blood mononuclear cells activated by treatment with concanavalin A and phorbol myristate acetate) cDNA library using methods which are selective for cDNAs encoding secreted proteins (see U.S. Pat. No. 5,536,637). This cDNA clone was then used to isolate G86_{—}2 from a human adult blood (peripheral blood mononuclear cells activated by treatment with phytohemagglutinin, phorbol myristate acetate, and mixed lymphocyte reaction) cDNA library. G86_{—}2 is a full-length clone, including the entire coding sequence of a secreted protein (also referred to herein as “G86_{—}2 protein”).

The nucleotide sequence of G86_{—}2 as presently determined is reported in SEQ ID NO:23, and includes a poly(A) tail. What applicants presently believe to be the proper reading frame and the predicted amino acid sequence of the G86_{—}2 protein corresponding to the foregoing nucleotide sequence is reported in SEQ ID NO:24. Amino acids 83 to 95 of SEQ ID NO:24 are a predicted leader/signal sequence, with the predicted mature amino acid sequence beginning at amino acid 96. Due to the hydrophobic nature of the predicted leader/signal sequence, it is likely to act as a transmembrane domain should the predicted leader/signal sequence not be separated from the remainder of the G86_{—}2 protein.

The EcoRI/NotI restriction fragment obtainable from the deposit containing clone G86_{—}2 should be approximately 3149 bp.

The nucleotide sequence disclosed herein for G86_{—}2 was searched against the GenBank and GeneSeq nucleotide sequence databases using BLASTN/BLASTX and FASTA search protocols. G86_{—}2 demonstrated at least some similarity with sequences identified as N25312 (yw52g09.s1 Homo sapiens cDNA clone 255904 3′) and R61399 (yh15h07.s1 Homo sapiens cDNA clone 37992 3′) The predicted amino acid sequence disclosed herein for G86_{—}2 was searched against the GenPept and GeneSeq amino acid sequence databases using the BLASTX search protocol. The predicted G86_{—}2 protein demonstrated at least some similarity with sequences identified as Z48639 (unknown [Saccharomyces cerevisiae]). Based upon sequence similarity, G86_{—}2 proteins and each similar protein or peptide may share at least some activity. The TopPredII computer program predicts six potential transmembrane domains within the G86_{—}2 protein sequence, centered around amino acids 90, 360, 520, 720, 800, and 820 of SEQ ID NO:24, respectively.

G86_{—}2 protein was expressed in a COS cell expression system, and an expressed protein band of approximately 110 kDa was detected in conditioned medium using SDS polyacrylamide gel electrophoresis.

Clone “H83_{—}22”

A polynucleotide of the present invention has been identified as clone “H83_{—}22”. H83_{—}22 was isolated from a human adult blood (peripheral blood mononuclear cells activated by treatment with phytohemagglutinin, phorbol myristate acetate, and mixed lymphocyte reaction) cDNA library using methods which are selective for cDNAs encoding secreted proteins (see U.S. Pat. No. 5,536,637). H83_{—}22 is a full-length clone, including the entire coding sequence of a secreted protein (also referred to herein as “H83_{—}22 protein”).

The nucleotide sequence of H83_{—}22 as presently determined is reported in SEQ ID NO:25, and includes a poly(A) tail. What applicants presently believe to be the proper reading frame and the predicted amino acid sequence of the H83_{—}22 protein corresponding to the foregoing nucleotide sequence is reported in SEQ ID NO:26. The extreme N-terminus of the H83_{—}22 protein (from about amino acid 4 to about amino acid 16) is a predicted leader/signal sequence, with the predicted mature amino acid sequence beginning at about amino acid 17. Due to the hydrophobic nature of the predicted leader/signal sequence, it is likely to act as a transmembrane domain should the predicted leader/signal sequence not be separated from the remainder of the G86_{—}2 protein.

The EcoRI/NotI restriction fragment obtainable from the deposit containing clone H83_{—}22 should be approximately 1107 bp.

The nucleotide sequence disclosed herein for H83_{—}22 was searched against the GenBank and GeneSeq nucleotide sequence databases using BLASTN/BLASTX and FASTA search protocols. H83_{—}22 demonstrated at least some similarity with sequences identified as AA004281 (zh88g07.r1 Soares fetal liver spleen 1NFLS S1 Homo sapiens cDNA clone 428412 5′), AA005382 (zh88g07.s1 Soares fetal liver spleen 1NFLS S1 Homo sapiens cDNA clone 428412 3′ similar to SW KLK9_MOUSE P15949 GLANDULAR KALLIKREIN K9 PRECURSOR), N71407 (Human serine protease gene), Q63795 (Bovine trypsinogen gene), Q66910 (Serine protease Met-ase), and U26174 (Human pre-granzyme 3 mRNA, complete cds). The predicted amino acid sequence disclosed herein for H83_{—}22 was searched against the GenPept and GeneSeq amino acid sequence databases using the BLASTX search protocol. The predicted H83_{—}22 protein demonstrated at least some similarity to sequences identified as L19694 (tryptase 2 [Rattus norvegicus]), P71672 (Human serine protease), and U26174 (pre-granzyme 3 [Homo sapiens]). Pre-granzyme 3 (U26174) is a serine protease produced by cytolytic “killer” T-lymphocytes and found in intracellular granules that are then exocytosed upon target aquisition. The amino terminus of pre-granzyme 3 is utilized as a signal peptide. Based upon sequence similarity, H83_{—}22 proteins and each similar protein or peptide may share at least some activity. The TopPredII computer program predicts two potential transmembrane domains within the H83_{—}22 protein sequence, one at the extreme N-terminus of the H83_{—}22 protein and another around amino acid 230 of SEQ ID NO:26.

H83_{—}22 protein was expressed in a COS cell expression system, and an expressed protein band of approximately 29 kDa was detected in conditioned medium and membrane fractions using SDS polyacrylamide gel electrophoresis.

Clone “H298_{—}23”

A polynucleotide of the present invention has been identified as clone “H298_{—}23”. H298_{—}23 was isolated from a human adult blood (peripheral blood mononuclear cells activated by treatment with phytohemagglutinin, phorbol myristate acetate, and mixed lymphocyte reaction) cDNA library using methods which are selective for cDNAs encoding secreted proteins (see U.S. Pat. No. 5,536,637). H298_{—}23 is a full-length clone, including the entire coding sequence of a secreted protein (also referred to herein as “H298_{—}23 protein”).

The nucleotide sequence of H298_{—}23 as presently determined is reported in SEQ ID NO:27, and includes a poly(A) tail. What applicants presently believe to be the proper reading frame and the predicted amino acid sequence of the H298_{—}23 protein corresponding to the foregoing nucleotide sequence is reported in SEQ ID NO:28. Amino acids 38 to 50 of SEQ ID NO:28 are a predicted leader/signal sequence, with the predicted mature amino acid sequence beginning at amino acid 51. Due to the hydrophobic nature of the predicted leader/signal sequence, it is likely to act as a transmembrane domain should the predicted leader/signal sequence not be separated from the remainder of the H298_{—}23 protein. H298_{—}23 appears to be a splice variant of clone G52_{—}24 described above.

The EcoRI/NotI restriction fragment obtainable from the deposit containing clone H298_{—}23 should be approximately 2300 bp.

The nucleotide sequence disclosed herein for H298_{—}23 was searched against the GenBank and GeneSeq nucleotide sequence databases using BLASTN/BLASTX and FASTA search protocols. H298_{—}23 demonstrated at least some similarity with sequences identified as AA029932 (zk08e11.r1 Soares pregnant uterus NbHPU Homo sapiens cDNA clone 469964 5′ similar to SW:CD69_HUMAN Q07108 EARLY ACTIVATION ANTIGEN CD69), L17895 (Human STS UT1833, sequence tagged site), M23613 (Human nucleophosmin mRNA, complete cds), T91294 (Human G52_{—}24 secreted protein cDNA internal fragment), and X96719 (H. sapiens mRNA for AICL (activation-induced C-type lectin)). The predicted amino acid sequence disclosed herein for H298_{—}23 was searched against the GenPept and GeneSeq amino acid sequence databases using the BLASTX search protocol. The predicted H298_{—}23 protein demonstrated at least some similarity to sequences identified as A10540 (human low affinity Fc-epsilon-receptor), M23613 (nucleophosmin [Homo sapiens]), R54659 (Human CD69), W27288 (Human G52_{—}24 secreted protein), and X96719 (C-Type lectin [Homo sapiens]). Based upon sequence similarity, H298_{—}23 proteins and each similar protein or peptide may share at least some activity.

Clone “H849_{—}24”

A polynucleotide of the present invention has been identified as clone “H849_{—}24”. H849_{—}24 was isolated from a human adult blood (peripheral blood mononuclear cells activated by treatment with phytohemagglutinin, phorbol myristate acetate, and mixed lymphocyte reaction) cDNA library using methods which are selective for cDNAs encoding secreted proteins (see U.S. Pat. No. 5,536,637). H849_{—}24 is a full-length clone, including the entire coding sequence of a secreted protein (also referred to herein as “H849_{—}24 protein”).

The nucleotide sequence of H849_{—}24 as presently determined is reported in SEQ ID NO:29, and includes a poly(A) tail. What applicants presently believe to be the proper reading frame and the predicted amino acid sequence of the H849_{—}24 protein corresponding to the foregoing nucleotide sequence is reported in SEQ ID NO:30. Amino acids 7 to 19 of SEQ ID NO:30 are a predicted leader/signal sequence, with the predicted mature amino acid sequence beginning at amino acid 20. Due to the hydrophobic nature of the predicted leader/signal sequence, it is likely to act as a transmembrane domain should the predicted leader/signal sequence not be separated from the remainder of the H849_{—}24 protein.

The EcoRI/NotI restriction fragment obtainable from the deposit containing clone H849_{—}24 should be approximately 1053 bp.

The nucleotide sequence disclosed herein for H849_{—}24 was searched against the GenBank and GeneSeq nucleotide sequence databases using BLASTN/BLASTX and FASTA search protocols. H849_{—}24 demonstrated at least some similarity with sequences identified as T86917 (yd86g12.r1 Homo sapiens cDNA clone 115174 5′ similar to SP MYP0_HETFR P20938 MYELIN P0 PROTEIN). The predicted amino acid sequence disclosed herein for H849_{—}24 was searched against the GenPept and GeneSeq amino acid sequence databases using the BLASTX search protocol. The predicted H849_{—}24 protein demonstrated at least some similarity to sequences identified as L24893 (myelin protein zero (Homo sapiens]). Based upon sequence similarity, H849_{—}24 proteins and each similar protein or peptide may share at least some activity. The TopPredII computer program predicts an additional potential transmembrane domain within the H849_{—}24 protein sequence, centered around amino acid 160 of SEQ ID NO:30.

H849_{—}24 protein was expressed in a COS cell expression system, and an expressed protein band of approximately 27 kDa was detected in membrane fractions using SDS polyacrylamide gel electrophoresis.

Clone “H905_{—}107”

A polynucleotide of the present invention has been identified as clone “H905_{—}107”. H905_{—}107 was isolated from a human adult blood (peripheral blood mononuclear cells activated by treatment with phytohemagglutinin, phorbol myristate acetate, and mixed lymphocyte reaction) cDNA library using methods which are selective for cDNAs encoding secreted proteins (see U.S. Pat. No. 5,536,637). H905_{—}107 is a full-length clone, including the entire coding sequence of a secreted protein (also referred to herein as “H905_{—}107 protein”).

The nucleotide sequence of H905_{—}107 as presently determined is reported in SEQ ID NO:31, and includes a poly(A) tail. What applicants presently believe to be the proper reading frame and the predicted amino acid sequence of the H905_{—}107 protein corresponding to the foregoing nucleotide sequence is reported in SEQ ID NO:32. Amino acids 21 to 33 of SEQ ID NO:32 are a possible leader/signal sequence, with the predicted mature amino acid sequence beginning at amino acid 34. Due to the hydrophobic nature of the possible leader/signal sequence, it is likely to act as a transmembrane domain should it not be separated from the remainder of the H905_{—}107 protein.

The EcoRI/NotI restriction fragment obtainable from the deposit containing clone H905_{—}107 should be approximately 730 bp.

The nucleotide sequence disclosed herein for H905_{—}107 was searched against the GenBank and GeneSeq nucleotide sequence databases using BLASTN/BLASTX and FASTA search protocols. H905_{—}107 demonstrated at least some similarity with sequences identified as AA864874 (oh03d08.s1 NCI_CGAP_Kid3 Homo sapiens cDNA clone IMAGE 1456719 3′ similar to TR Q92478 Q92478 C-TYPE LECTIN; contains Alu repetitive element; mRNA sequence), H11808 (ym11e03.r1 Homo sapiens cDNA clone 47481 5′ similar to SP LEC1_FOWPM P14370 HEPATIC LECTIN HOMOLOG), Q04525 (Total base sequence of rice plant chloroplast DNA), and X96719 (H. sapiens mRNA for AICL (activation-induced C-type lectin)). The predicted amino acid sequence disclosed herein for H905_{—}107 was searched against the GenPept and GeneSeq amino acid sequence databases using the BLASTX search protocol. The predicted H905_{—}107 protein demonstrated at least some similarity to sequences identified as R54659 (Human CD69) and X96719 (C-Type lectin [Homo sapiens]). Based upon sequence similarity, H905_{—}107 proteins and each similar protein or peptide may share at least some activity. The TopPredII computer program predicts a potential transmembrane domain within the H905_{—}107 protein sequence centered around amino acid 25 of SEQ ID NO:32.

H905_{—}107 protein was expressed in a COS cell expression system, and an expressed protein band of approximately 27 kDa was detected in membrane fractions using SDS polyacrylamide gel electrophoresis.

Clone “H1075_{—}1”

A polynucleotide of the present invention has been identified as clone “H1075_{—}1”. H1075_{—}1 was isolated from a human adult blood (peripheral blood mononuclear cells activated by treatment with phytohemagglutinin, phorbol myristate acetate, and mixed lymphocyte reaction) cDNA library using methods which are selective for cDNAs encoding secreted proteins (see U.S. Pat. No. 5,536,637). H1075_{—}1 is a full-length clone, including the entire coding sequence of a secreted protein (also referred to herein as “H1075_{—}1 protein”).

The nucleotide sequence of H1075_{—}1 as presently determined is reported in SEQ ID NO:33, and includes a poly(A) tail. What applicants presently believe to be the proper reading frame and the predicted amino acid sequence of the H1075_{—}1 protein corresponding to the foregoing nucleotide sequence is reported in SEQ ID NO:34. Amino acids 7 to 19 of SEQ ID NO:34 are a predicted leader/signal sequence, with the predicted mature amino acid sequence beginning at amino acid 20. Due to the hydrophobic nature of the predicted leader/signal sequence, it is likely to act as a transmembrane domain should the predicted leader/signal sequence not be separated from the remainder of the H1075_{—}1 protein.

The EcoRI/NotI restriction fragment obtainable from the deposit containing clone H1075_{—}1 should be approximately 1080 bp.

The nucleotide sequence disclosed herein for H1075_{—}1 was searched against the GenBank and GeneSeq nucleotide sequence databases using BLASTN/BLASTX and FASTA search protocols. H1075_{—}1 demonstrated at least some similarity with sequences identified as A18921 (Synthetic human cDNA of gene HE1), AA156678 (zl19a06.r1 Soares pregnant uterus NbHPU Homo sapiens cDNA clone 502354 5′ similar to TR G37477 G37477 TISSUE SPECIFIC MRNA), AA188372 (zp72f06.s1 Stratagene endothelial cell 937223 Homo sapiens cDNA clone 625763 3′), D82271 (similar to none), N30130 (yx81b05.s1 Homo sapiens cDNA clone 268113 3′ similar to SP PIR S25641 S25641 hypothetical protein—human), Q13125 (HE1 epididymis-specific DNA), T20811 (Human gene signature HUMGS02059), T86063 (yd62h04.r1 Homo sapiens cDNA clone 112855 5′ similar to SP:S25641 S25641 HYPOTHETICAL PROTEIN), U25748 (Pan troglodytes epididymal secretory protein precursor (EPI-1) mRNA, complete cds), and X67698 (H. sapiens tissue specific mRNA). The predicted amino acid sequence disclosed herein for H1075_{—}1 was searched against the GenPept and GeneSeq amino acid sequence databases using the BLASTX search protocol. The predicted H1075_{—}1 protein demonstrated at least some similarity to sequences identified as A18921 (tissue-specific secretory protein [unidentified]), R13327 (HE1 epididymis-specific protein), and X67698 (orf [Homo sapiens]). Based upon sequence similarity, H1075_{—}1 proteins and each similar protein or peptide may share at least some activity.

Clone “J59_{—}41”

A polynucleotide of the present invention has been identified as clone “J59_{—}41”. J59_{—}41 was isolated from a human adult blood (peripheral blood mononuclear cells activated by treatment with phytohemagglutinin, phorbol myristate acetate, and mixed lymphocyte reaction) cDNA library using methods which are selective for cDNAs encoding secreted proteins (see U.S. Pat. No. 5,536,637). J59_{—}41 is a full-length clone, including the entire coding sequence of a secreted protein (also referred to herein as “J59_{—}41 protein”).

The nucleotide sequence of J59_{—}41 as presently determined is reported in SEQ ID NO:35, and includes a poly(A) tail. What applicants presently believe to be the proper reading frame and the predicted amino acid sequence of the J59_{—}41 protein corresponding to the foregoing nucleotide sequence is reported in SEQ ID NO:36. (Were a frameshift to occur between about nucleotide 620 to about nucleotide 690 of SEQ ID NO:35, the resulting nucleotide sequence could encode an amino acid sequence of about 336 amino acids.) Amino acids 172 to 184 of SEQ ID NO:36 are a predicted leader/signal sequence, with the predicted mature amino acid sequence beginning at amino acid 185. Due to the hydrophobic nature of the predicted leader/signal sequence, it is likely to act as a transmembrane domain should the predicted leader/signal sequence not be separated from the remainder of the J59_{—}41 protein.

The EcoRI/NotI restriction fragment obtainable from the deposit containing clone J59_{—}41 should be approximately 1600 bp.

The nucleotide sequence disclosed herein for J59_{—}41 was searched against the GenBank and GeneSeq nucleotide sequence databases using BLASTN/BLASTX and FASTA search protocols. J59_{—}41 demonstrated at least some similarity with sequences identified as AA075033 (zm85a11.r1 Stratagene ovarian cancer (#937219) Homo sapiens cDNA clone 544700 5′ similar to WP F54E7.1 CE01312), AA085746 (zk71b12.s1 Soares pregnant uterus NbHPU Homo sapiens cDNA clone 488255 3′, mRNA sequence), R21739 (yh21d06.s1 Homo sapiens cDNA clone 130379 3′), and W07150 (za92e11.r1 Soares fetal lung NbHL19W Homo sapiens cDNA clone 300044 5′, mRNA sequence). The predicted amino acid sequence disclosed herein for J59_{—}41 was searched against the GenPept and GeneSeq amino acid sequence databases using the BLASTX search protocol. The predicted J59_{—}41 protein demonstrated at least some similarity to sequences identified as U00067 (No definition line found [Caenorhabditis elegans]). Based upon sequence similarity, J59_{—}41 proteins and each similar protein or peptide may share at least some activity. The TopPredII computer program predicts four additional potential transmembrane domains within the J59_{—}41 protein sequence, centered around amino acids 56, 88, 123, and 201 of SEQ ID NO:36, respectively.

Clone “J143_{—}1”

A polynucleotide of the present invention has been identified as clone “J143_{—}1”. J143_{—}1 was isolated from a human adult blood (peripheral blood mononuclear cells activated by treatment with phytohemagglutinin, phorbol myristate acetate, and mixed lymphocyte reaction) cDNA library using methods which are selective for cDNAs encoding secreted proteins (see U.S. Pat. No. 5,536,637). J143_{—}1 is a full-length clone, including the entire coding sequence of a secreted protein (also referred to herein as “J143_{—}1 protein”).

The nucleotide sequence of J143_{—}1 as presently determined is reported in SEQ ID NO:37, and includes a poly(A) tail. What applicants presently believe to be the proper reading frame and the predicted amino acid sequence of the J143_{—}1 protein corresponding to the foregoing nucleotide sequence is reported in SEQ ID NO:38. Amino acids 61 to 73 of SEQ ID NO:38 are a predicted leader/signal sequence, with the predicted mature amino acid sequence beginning at amino acid 74. Due to the hydrophobic nature of the predicted leader/signal sequence, it is likely to act as a transmembrane domain should the predicted leader/signal sequence not be separated from the remainder of the J143_{—}1 protein.

The EcoRI/NotI restriction fragment obtainable from the deposit containing clone J143_{—}1 should be approximately 1700 bp.

The nucleotide sequence disclosed herein for J143_{—}1 was searched against the GenBank and GeneSeq nucleotide sequence databases using BLASTN/BLASTX and FASTA search protocols. J143_{—}1 demonstrated at least some similarity with sequences identified as AA115640 (zl88d10.s1 Stratagene colon (#937204) Homo sapiens cDNA clone 511699 3′, mRNA sequence), AA189196 (mu46h09.r1 Soares mouse lymph node NbMLN Mus musculus cDNA clone 6424975′, mRNA sequence), AB011118 (Homo sapiens mRNA for KIAA0546 protein, partial cds), and R60365 (yh04a07.r1 Homo sapiens cDNA clone 41951 5′). The predicted amino acid sequence disclosed herein for J143_{—}1 was searched against the GenPept and GeneSeq amino acid sequence databases using the BLASTX search protocol. The predicted J143_{—}1 protein demonstrated at least some similarity to sequences identified as AB011118 (KIAA0546 protein [Homo sapiens]). Based upon sequence similarity, J143_{—}1 proteins and each similar protein or peptide may share at least some activity.

Clone “J218_{—}15”

A polynucleotide of the present invention has been identified as clone “J218_{—}15”. J218_{—}15 was isolated from a human adult blood (peripheral blood mononuclear cells activated by treatment with phytohemagglutinin, phorbol myristate acetate, and mixed lymphocyte reaction) cDNA library using methods which are selective for cDNAs encoding secreted proteins (see U.S. Pat. No. 5,536,637). J218_{—}15 is a full-length clone, including the entire coding sequence of a secreted protein (also referred to herein as “J218_{—}15 protein”).

The nucleotide sequence of J218_{—}15 as presently determined is reported in SEQ ID NO:39, and includes a poly(A) tail. What applicants presently believe to be the proper reading frame and the predicted amino acid sequence of the J218_{—}15 protein corresponding to the foregoing nucleotide sequence is reported in SEQ ID NO:40. Amino acids 88 to 100 of SEQ ID NO:40 are a predicted leader/signal sequence, with the predicted mature amino acid sequence beginning at amino acid 101. Due to the hydrophobic nature of the predicted leader/signal sequence, it is likely to act as a transmembrane domain should the predicted leader/signal sequence not be separated from the remainder of the J218_{—}15 protein.

The EcoRI/NotI restriction fragment obtainable from the deposit containing clone J218_{—}15 should be approximately 2600 bp.

The nucleotide sequence disclosed herein for J218_{—}15 was searched against the GenBank and GeneSeq nucleotide sequence databases using BLASTN/BLASTX and FASTA search protocols. J218_{—}15 demonstrated at least some similarity with sequences identified as AA009923 (zi07e01.r1 Soares fetal liver spleen 1NFLS S1 Homo sapiens cDNA clone 430104 5′), AB000280 (Rat mRNA for peptide/histidine transporter, complete cds), H65908 (yr69b03.r1 Homo sapiens cDNA clone 210509 5′ similar to SP:PEPT_RABIT P36836 OLIGOPEPTIDE TRANSPORTER), H73031 (ys10b07.r1 Homo sapiens cDNA clone 214357 5′), R51893 (yg78d02.s1 Homo sapiens cDNA clone 39680 3′), T11631 (Human leukotrine C4 synthase coding sequence), and W53019 (zc48d11.r1 Soares senescent fibroblasts NbHSF Homo sapiens cDNA clone 325557 5′ similar to SW PT2B_ARATH P46032 PEPTIDE TRANSPORTER PTR2-B; mRNA sequence). The predicted amino acid sequence disclosed herein for J218_{—}15 was searched against the GenPept and GeneSeq amino acid sequence databases using the BLASTX search protocol. The predicted J218_{—}15 protein demonstrated at least some similarity to sequences identified as AB000280 (peptide/histidine transporter [Rattus norvegicus]), R84891 (Peptide transport protein ATPTR2Ap), R84892 (Peptide transport protein ATPTR2 Bp), and U01171 (similar to S. cerevisiae PTR2 gene, GenBank Accession Number L11994 [Arabidopsis thaliana]). Based upon sequence similarity, J218_{—}15 proteins and each similar protein or peptide may share at least some activity.

Clone “M8_{—}2”

A polynucleotide of the present invention has been identified as clone “M8_{—}2”. M8_{—}2 was isolated from a human adult neural tissue (glioblastoma line T98G) cDNA library using methods which are selective for cDNAs encoding secreted proteins (see U.S. Pat. No. 5,536,637). M8_{—}2 is a full-length clone, including the entire coding sequence of a secreted protein (also referred to herein as “M8_{—}2 protein”).

The nucleotide sequence of M8_{—}2 as presently determined is reported in SEQ ID NO:41, and includes a poly(A) tail. What applicants presently believe to be the proper reading frame and the predicted amino acid sequence of the M8_{—}2 protein corresponding to the foregoing nucleotide sequence is reported in SEQ ID NO:42.

The EcoRI/NotI restriction fragment obtainable from the deposit containing clone M8_{—}2 should be approximately 650 bp.

The nucleotide sequence disclosed herein for M8_{—}2 was searched against the GenBank and GeneSeq nucleotide sequence databases using BLASTN/BLASTX and FASTA search protocols. M8_{—}2 demonstrated at least some similarity with sequences identified as R97122 (yq58d01.r1 Homo sapiens cDNA clone 199969 5′ similar to contains MSR1 repetitive element), U23863 (Human clone mcag36 chromosome 1 CAG repeat region), U29397 (Rattus norvegicus plasma membrane Ca2+ ATPase isoform 3 (PMCA3) gene, 5′ flanking region), and X59828 (Human chromosome 22 flanking hypervariable simple repeat DNA (clone HZREP42)). Based upon sequence similarity, M8_{—}2 proteins and each similar protein or peptide may share at least some activity. The nucleotide sequence of M8_{—}2 indicates that it may contain one or more simple CCA repeat regions.

Clone “M97_{—}2”

A polynucleotide of the present invention has been identified as clone “M97_{—}2”. M97_{—}2 was isolated from a human adult neural tissue (glioblastoma line T98G) cDNA library using methods which are selective for cDNAs encoding secreted proteins (see U.S. Pat. No. 5,536,637). M97_{—}2 is a full-length clone, including the entire coding sequence of a secreted protein (also referred to herein as “M97_{—}2 protein”).

The nucleotide sequence of M97_{—}2 as presently determined is reported in SEQ ID NO:43, and includes a poly(A) tail. What applicants presently believe to be the proper reading frame and the predicted amino acid sequence of the M97_{—}2 protein corresponding to the foregoing nucleotide sequence is reported in SEQ ID NO:44. Amino acids 19 to 31 of SEQ ID NO:44 are a predicted leader/signal sequence, with the predicted mature amino acid sequence beginning at amino acid 32. Due to the hydrophobic nature of the predicted leader/signal sequence, it is likely to act as a transmembrane domain should the predicted leader/signal sequence not be separated from the remainder of the M97_{—}2 protein.

The EcoRI/NotI restriction fragment obtainable from the deposit containing clone M97_{—}2 should be approximately 500 bp.

The nucleotide sequence disclosed herein for M97_{—}2 was searched against the GenBank and GeneSeq nucleotide sequence databases using BLASTN/BLASTX and FASTA search protocols. M97_{—}2 demonstrated at least some similarity with sequences identified as F09835 (H. sapiens partial cDNA sequence; clone c-37a09), N35995 (yy31e07.s1 Homo sapiens cDNA clone 272868 3′), and Z25379 (H. sapiens partial cDNA sequence; clone C6F07; version 1; strand(+), single read). Based upon sequence similarity, M97_{—}2 proteins and each similar protein or peptide may share at least some activity.

Clone “O238_{—}1”

A polynucleotide of the present invention has been identified as clone “O238_{—}1”. O238_{—}1 was isolated from a human adult blood (dendritic cells) cDNA library using methods which are selective for cDNAs encoding secreted proteins (see U.S. Pat. No. 5,536,637). O238_{—}1 is a full-length clone, including the entire coding sequence of a secreted protein (also referred to herein as “O238_{—}1 protein”).

The nucleotide sequence of O238_{—}1 as presently determined is reported in SEQ ID NO:45. What applicants presently believe to be the proper reading frame and the predicted amino acid sequence of the O238_{—}1 protein corresponding to the foregoing nucleotide sequence is reported in SEQ ID NO:46. Amino acids 26 to 38 of SEQ ID NO:46 are a predicted leader/signal sequence, with the predicted mature amino acid sequence beginning at amino acid 39. Due to the hydrophobic nature of the predicted leader/signal sequence, it is likely to act as a transmembrane domain should the predicted leader/signal sequence not be separated from the remainder of the O238_{—}1 protein.

The EcoRI/NotI restriction fragment obtainable from the deposit containing clone O238_{—}1 should be approximately 664 bp.

The nucleotide sequence disclosed herein for O238_{—}1 was searched against the GenBank and GeneSeq nucleotide sequence databases using BLASTN/BLASTX and FASTA search protocols. O238_{—}1 demonstrated at least some similarity with sequences identified as AA515323 (ng71a07.s1 NCI_CGAP_Lip2 Homo sapiens cDNA clone 940212 similar to TR G506861 G506861 BST-2), D28137 (Human mRNA for BST-2, complete cds), N88483 (K3493F Fetal heart, Lambda ZAP Express Homo sapiens cDNA clone K3493 5′ similar to BST-2), and Q89606 (Human membrane pre-B cell growth enhancing polypeptide cDNA). The predicted amino acid sequence disclosed herein for O238_{—}1 was searched against the GenPept and GeneSeq amino acid sequence databases using the BLASTX search protocol. The predicted O238_{—}1 protein demonstrated at least some similarity to sequences identified as D28137 (BST-2 [Homo sapiens]) and R72703 (Human membrane polypeptide for enhancing pre-B cell growth). Based upon sequence similarity, O238_{—}1 proteins and each similar protein or peptide may share at least some activity. O238_{—}1 protein was expressed in a COS cell expression system, and an expressed protein band of approximately 20 kDa was detected in membrane fractions using SDS polyacrylamide gel electrophoresis.

Clone “S185_{—}2”

A polynucleotide of the present invention has been identified as clone “S185_{—}2”. A cDNA clone was first isolated from a human adult neural tissue (glioblastoma line TG-1) cDNA library using methods which are selective for cDNAs encoding secreted proteins (see U.S. Pat. No. 5,536,637). This cDNA clone was then used to isolate S185-2 from a human adult neural tissue (glioblastoma line T98G) cDNA library. S185_{—}2 is a full-length clone, including the entire coding sequence of a secreted protein (also referred to herein as “S185_{—}2 protein”).

The nucleotide sequence of S185_{—}2 as presently determined is reported in SEQ ID NO:47, and includes a poly(A) tail. What applicants presently believe to be the proper reading frame and the predicted amino acid sequence of the S185_{—}2 protein corresponding to the foregoing nucleotide sequence is reported in SEQ ID NO:48. Amino acids 34 to 46 of SEQ ID NO:48 are a predicted leader/signal sequence, with the predicted mature amino acid sequence beginning at amino acid 47. Due to the hydrophobic nature of the predicted leader/signal sequence, it is likely to act as a transmembrane domain should the predicted leader/signal sequence not be separated from the remainder of the S185_{—}2 protein.

The EcoRI/NotI restriction fragment obtainable from the deposit containing clone S185_{—}2 should be approximately 2120 bp.

The nucleotide sequence disclosed herein for S185_{—}2 was searched against the GenBank and GeneSeq nucleotide sequence databases using BLASTN/BLASTX and FASTA search protocols. S185_{—}2 demonstrated at least some similarity with sequences identified as AA114093 (zn66e11.r1 Stratagene HeLa cell s3 937216 Homo sapiens cDNA clone 563180 5′), D89050 (Human mRNA for lectin-like oxidized LDL receptor, complete cds), L47482 (Human chromosome X STS sWXD2062, single read, sequence tagged site), N51624 (yy98h04.s1 Homo sapiens cDNA clone 281623 3′ similar to contains L1.t1 L1 repetitive element), R31604 (yh76h02.s1 Homo sapiens cDNA clone 135699 3′), and T32578 (Low density lipoprotein receptor coding sequence). The predicted amino acid sequence disclosed herein for S185_{—}2 was searched against the GenPept and GeneSeq amino acid sequence databases using the BLASTX search protocol. The predicted S185_{—}2 protein demonstrated at least some similarity to sequences identified as D89050 (lectin-like oxidized LDL receptor (Homo sapiens]) and R99588 (Low density lipoprotein receptor). Based upon sequence similarity, S185_{—}2 proteins and each similar protein or peptide may share at least some activity. The nucleotide sequence of S185_{—}2 indicates that it may contain one or more repetitive elements.

Clone “AJ147_{—}1”

A polynucleotide of the present invention has been identified as clone “AJ147_{—}1”. AJ147_{—}1 was isolated from a human adult testes cDNA library using methods which are selective for cDNAs encoding secreted proteins (see U.S. Pat. No. 5,536,637). AJ147_{—}1 is a full-length clone, including the entire coding sequence of a secreted protein (also referred to herein as “AJ147_{—}1 protein”).

The nucleotide sequence of AJ147_{—}1 as presently determined is reported in SEQ ID NO:49, and includes a poly(A) tail. What applicants presently believe to be the proper reading frame and the predicted amino acid sequence of the AJ147_{—}1 protein corresponding to the foregoing nucleotide sequence is reported in SEQ ID NO:50. Amino acids 7 to 19 of SEQ ID NO:50 are a predicted leader/signal sequence, with the predicted mature amino acid sequence beginning at amino acid 20. Due to the hydrophobic nature of the predicted leader/signal sequence, it is likely to act as a transmembrane domain should the predicted leader/signal sequence not be separated from the remainder of the AJ147_{—}1 protein.

The EcoRI/NotI restriction fragment obtainable from the deposit containing clone AJ147_{—}1 should be approximately 500 bp.

The nucleotide sequence disclosed herein for AJ147_{—}1 was searched against the GenBank and GeneSeq nucleotide sequence databases using BLASTN/BLASTX and FASTA search protocols. AJ147_{—}1 demonstrated at least some similarity with sequences identified as AA279490 (zs85h03.s1 NCI_CGAP_GCB1 Homo sapiens cDNA clone IMAGE:704309 3′ similar to TR:G473720 G473720 CALMEGIN(MEG 1); mRNA sequence), AA355553 (EST63972 Jurkat T-cells VI Homo sapiens cDNA 5′ end similar to similar to calnexin), D14117 (Mouse mRNA for calmegin (Meg 1), complete cds), D86322 (Homo sapiens mRNA for calmegin, complete cds), and Q84723 (Calnexin DNA sequence). The predicted amino acid sequence disclosed herein for AJ147_{—}1 was searched against the GenPept and GeneSeq amino acid sequence databases using the BLASTX search protocol. The predicted AJ147_{—}1 protein demonstrated at least some similarity to sequences identified as D14117 (calmegin(Meg 1) [Mus musculus]), D86322 (calmegin [Homo sapiens]), and R71094 (Calnexin sequence). Based upon sequence similarity, AJ147_{—}1 proteins and each similar protein or peptide may share at least some activity. The TopPredII computer program predicts a potential transmembrane domain within the AJ147_{—}1 protein sequence centered around amino acid 20 of SEQ ID NO:50.

Clone “AM262_{—}11”

A polynucleotide of the present invention has been identified as clone “AM262_{—}11”. AM262_{—}11 was isolated from a human fetal kidney cDNA library using methods which are selective for cDNAs encoding secreted proteins (see U.S. Pat. No. 5,536,637). AM262_{—}11 is a full-length clone, including the entire coding sequence of a secreted protein (also referred to herein as “AM262_{—}11 protein”).

The nucleotide sequence of AM262_{—}11 as presently determined is reported in SEQ ID NO:51, and includes a poly(A) tail. What applicants presently believe to be the proper reading frame and the predicted amino acid sequence of the AM262_{—}11 protein corresponding to the foregoing nucleotide sequence is reported in SEQ ID NO:52. Amino acids 6 to 18 of SEQ ID NO:52 are a predicted leader/signal sequence, with the predicted mature amino acid sequence beginning at amino acid 19. Due to the hydrophobic nature of the predicted leader/signal sequence, it is likely to act as a transmembrane domain should the predicted leader/signal sequence not be separated from the remainder of the AM262_{—}11 protein.

The EcoRI/NotI restriction fragment obtainable from the deposit containing clone AM262_{—}11 should be approximately 500 bp.

The nucleotide sequence disclosed herein for AM262_{—}11 was searched against the GenBank and GeneSeq nucleotide sequence databases using BLASTN/BLASTX and FASTA search protocols. AM262_{—}11 demonstrated at least some similarity with sequences identified as A17786 (MCP-1 mRNA), D49372 (Human mRNA for eotaxin, complete cds), M24545 (Human monocyte chemotactic and activating factor (MCAF) mRNA, complete cds), S67954 (monocyte chemoattractant protein-2 {clone c11/1} [cattle, blood PMNLs, mRNA]), U34780 (Human eotaxin precursor gene, complete cds), U46573 (Human eotaxin precursor mRNA, complete cds), and Z69291 (H. sapiens mRNA for CC-chemokine). The predicted amino acid sequence disclosed herein for AM262_{—}11 was searched against the GenPept and GeneSeq amino acid sequence databases using the BLASTX search protocol. The predicted AM262_{—}11 protein demonstrated at least some similarity to sequences identified as A17786 (MCP-1 gene product [unidentified]), D49372 (eotaxin [Homo sapiens]), and U34780 (eotaxin precursor [Homo sapiens]). Based upon sequence similarity, AM262_{—}11 proteins and each similar protein or peptide may share at least some activity.

AM262_{—}11 protein was expressed in a COS cell expression system, and an expressed protein band of approximately 7 kDa was detected in conditioned medium using SDS polyacrylamide gel electrophoresis.

Clone “AR28_{—}1”

A polynucleotide of the present invention has been identified as clone “AR28_{—}1”. AR28_{—}1 was isolated from a human adult retina cDNA library using methods which are selective for cDNAs encoding secreted proteins (see U.S. Pat. No. 5,536,637). AR28_{—}1 is a full-length clone, including the entire coding sequence of a secreted protein (also referred to herein as “AR28_{—}1 protein”).

The nucleotide sequence of AR28_{—}1 as presently determined is reported in SEQ ID NO:53, and includes a poly(A) tail. What applicants presently believe to be the proper reading frame and the predicted amino acid sequence of the AR28_{—}1 protein corresponding to the foregoing nucleotide sequence is reported in SEQ ID NO:54. Amino acids 23 to 35 of SEQ ID NO:54 are a possible leader/signal sequence, with the predicted mature amino acid sequence beginning at amino acid 36. Due to the hydrophobic nature of this possible leader/signal sequence, it is likely to act as a transmembrane domain should it not be separated from the remainder of the AR28_{—}1 protein.

The EcoRI/NotI restriction fragment obtainable from the deposit containing clone AR28_{—}1 should be approximately 2200 bp.

The nucleotide sequence disclosed herein for AR28_{—}1 was searched against the GenBank and GeneSeq nucleotide sequence databases using BLASTN/BLASTX and FASTA search protocols. AR28_{—}1 demonstrated at least some similarity with sequences identified as M37551 (Human AFP gene, intron with a partial Alu repeat), U14568 (***ALU WARNING: Human Alu-Sb subfamily consensus sequence), W28179 (43b5 Human retina cDNA randomly primed sublibrary Homo sapiens cDNA), and X76070 (H. sapiens IgK locus, clone cos211, DNA, sequence tagged site). The predicted amino acid sequence disclosed herein for AR28_{—}1 was searched against the GenPept and GeneSeq amino acid sequence databases using the BLASTX search protocol. The predicted AR28_{—}1 protein demonstrated at least some similarity to sequences identified as X63672 (polyhomeotic gene product [Drosophila melanogaster]). Based upon sequence similarity, AR28_{—}1 proteins and each similar protein or peptide may share at least some activity. The predicted AR28_{—}1 protein contains two motifs: a “eukaryotic thiol (cysteine) proteases active site” at residue 164 of SEQ ID NO:54 and a “binding-protein-dependent transport systems inner membrane component signature” at residue 232 of SEQ ID NO:54. The nucleotide sequence of AR28_{—}1 indicates that it may contain an Alu repetitive element.

Clone “AS86_{—}1”

A polynucleotide of the present invention has been identified as clone “AS86_{—}1”. AS86_{—}1 was isolated from a human fetal brain cDNA library using methods which are selective for cDNAs encoding secreted proteins (see U.S. Pat. No. 5,536,637). AS86_{—}1 is a full-length clone, including the entire coding sequence of a secreted protein (also referred to herein as “AS86_{—}1 protein”).

The nucleotide sequence of AS86_{—}1 as presently determined is reported in SEQ ID NO:55, and includes a poly(A) tail. What applicants presently believe to be the proper reading frame and the predicted amino acid sequence of the AS86_{—}1 protein corresponding to the foregoing nucleotide sequence is reported in SEQ ID NO:56. Amino acids 28 to 40 of SEQ ID NO:56 are a predicted leader/signal sequence, with the predicted mature amino acid sequence beginning at amino acid 41. Due to the hydrophobic nature of the predicted leader/signal sequence, it is likely to act as a transmembrane domain should the predicted leader/signal sequence not be separated from the remainder of the AS86_{—}1 protein.

The EcoRI/NotI restriction fragment obtainable from the deposit containing clone AS86_{—}1 should be approximately 2100 bp.

The nucleotide sequence disclosed herein for AS86_{—}1 was searched against the GenBank and GeneSeq nucleotide sequence databases using BLASTN/BLASTX and FASTA search protocols. AS86_{—}1 demonstrated at least some similarity with sequences identified as AA013474 (ze29b08.s1 Soares retina N2b4HR Homo sapiens cDNA clone 360375 3′), AA063495 (zf70b06.r1 Soares pineal gland N3HPG Homo sapiens cDNA clone 382259 5′ similar to contains Alu repetitive element; contains element MER37 repetitive element), and AA362774 (EST72562 Ovary II Homo sapiens cDNA 5′ end). Based upon sequence similarity, AS86_{—}1 proteins and each similar protein or peptide may share at least some activity. The TopPredII computer program predicts a potential transmembrane domain within the AS86_{—}1 protein sequence centered around amino acid 25 of SEQ ID NO:56. The nucleotide sequence of AS86_{—}1 indicates that it may contain an Alu repetitive element.

Clone “AS162_{—}1”

A polynucleotide of the present invention has been identified as clone “AS162_{—}1”. AS162_{—}1 was isolated from a human fetal brain cDNA library using methods which are selective for cDNAs encoding secreted proteins (see U.S. Pat. No. 5,536,637). AS162_{—}1 is a full-length clone, including the entire coding sequence of a secreted protein (also referred to herein as “AS162_{—}1 protein”).

The nucleotide sequence of AS162_{—}1 as presently determined is reported in SEQ ID NO:57, and includes a poly(A) tail. What applicants presently believe to be the proper reading frame and the predicted amino acid sequence of the AS162_{—}1 protein corresponding to the foregoing nucleotide sequence is reported in SEQ ID NO:58. Amino acids 16 to 28 of SEQ ID NO:58 are a possible leader/signal sequence, with the predicted mature amino acid sequence beginning at amino acid 29. Due to the hydrophobic nature of the predicted leader/signal sequence, it is likely to act as a transmembrane domain should the predicted leader/signal sequence not be separated from the remainder of the AS86_{—}1 protein.

The amino acid sequence of another protein that could be encoded by nucleotides 17 to 589 of AS162_{—}1 is reported in SEQ ID NO:310. Amino acids 38 to 50 of the protein of SEQ ID NO:310 are a possible leader/signal sequence, with the predicted mature amino acid sequence beginning at amino acid 51. Due to the hydrophobic nature of the predicted leader/signal sequence, it is likely to act as a transmembrane domain should this predicted leader/signal sequence not be separated from the remainder of the protein of SEQ ID NO:310. The TopPredII computer program predicts four potential transmembrane domains within the amino acid sequence of the protein of SEQ ID NO:310, centered around amino acids 20, 49, 99, and 143 of SEQ ID NO:310, respectively. If a frameshift were introduced into the nucleotide sequence of SEQ ID NO:57, the reading frames encoding the proteins of SEQ ID NO:58 and SEQ ID NO:310 could be joined into a single open reading frame.

The EcoRI/NotI restriction fragment obtainable from the deposit containing clone AS162_{—}1 should be approximately 1400 bp.

The nucleotide sequence disclosed herein for AS162_{—}1 was searched against the GenBank and GeneSeq nucleotide sequence databases using BLASTN/BLASTX and FASTA search protocols. AS162_{—}1 demonstrated at least some similarity with sequences identified as AA604143 (no69a08.s1 NCI_CGAP_AA1 Homo sapiens cDNA clone [MAGE 1112054) and R88809 (ym96e05.s1 Homo sapiens cDNA clone 166784 3′). The predicted amino acid sequence disclosed herein for AS162_{—}1 was searched against the GenPept and GeneSeq amino acid sequence databases using the BLASTX search protocol. The predicted AS162_{—}1 protein demonstrated at least some similarity to sequences identified as D87469 (Similar to D. melanogaster cadherin-related tumor suppressor [Homo sapiens]) and U39848 (similar to G-protein coupled receptors [Caenorhabditis elegans]). Based upon sequence similarity, AS162_{—}1 proteins and each similar protein or peptide may share at least some activity. The TopPredII computer program predicts two potential transmembrane domains within the AS162_{—}1 protein sequence, one centered around amino acid 24 and another around amino acid 66 of SEQ ID NO:58.

Clone “AS264_{—}3”

A polynucleotide of the present invention has been identified as clone “AS264_{—}3”. AS264_{—}3 was isolated from a human fetal brain cDNA library using methods which are selective for cDNAs encoding secreted proteins (see U.S. Pat. No. 5,536,637). AS264_{—}3 is a full-length clone, including the entire coding sequence of a secreted protein (also referred to herein as “AS264_{—}3 protein”).

The nucleotide sequence of AS264_{—}3 as presently determined is reported in SEQ ID NO:59, and includes a poly(A) tail. What applicants presently believe to be the proper reading frame and the predicted amino acid sequence of the AS264_{—}3 protein corresponding to the foregoing nucleotide sequence is reported in SEQ ID NO:60.

The EcoRI/NotI restriction fragment obtainable from the deposit containing clone AS264_{—}3 should be approximately 3300 bp.

The nucleotide sequence disclosed herein for AS264_{—}3 was searched against the GenBank and GeneSeq nucleotide sequence databases using BLASTN/BLASTX and FASTA search protocols. AS264_{—}3 demonstrated at least some similarity with sequences identified as AB007890 (Homo sapiens KIAA0430 mRNA, complete cds) and N57913 (yv61c05.s1 Homo sapiens cDNA clone 247208 3′ similar to contains Alu repetitive element). The predicted amino acid sequence disclosed herein for AS264_{—}3 was searched against the GenPept and GeneSeq amino acid sequence databases using the BLASTX search protocol. The predicted AS264_{—}3 protein demonstrated at least some similarity to sequences identified as AB00789 (KLAA0430 [Homo sapiens]), M91669 (autoantigen [Homo sapiens]), X89807 (FGF receptor 4 [Xenopus laevis]), and X92485 (pva1 gene product [Plasmodium vivax]). Based upon sequence similarity, AS264_{—}3 proteins and each similar protein or peptide may share at least some activity. The nucleotide sequence of AS264_{—}3 indicates that it may contain an Alu repetitive element.

Clone “AS268_{—}1”

A polynucleotide of the present invention has been identified as clone “AS268_{—}1”. AS268_{—}1 was isolated from a human fetal brain cDNA library using methods which are selective for cDNAs encoding secreted proteins (see U.S. Pat. No. 5,536,637). AS268_{—}1 is a full-length clone, including the entire coding sequence of a secreted protein (also referred to herein as “AS268_{—}1 protein”).

The nucleotide sequence of the 5′ portion of AS268_{—}1 as presently determined is reported in SEQ ID NO:61. What applicants presently believe is the proper reading frame for the coding region is indicated in SEQ ID NO:62. The predicted amino acid sequence of the AS268_{—}1 protein corresponding to the foregoing nucleotide sequence is reported in SEQ ID NO:62. Additional nucleotide sequence from the 3′ portion of AS268_{—}1, including a poly(A) tail, is reported in SEQ ID NO:63.

The EcoRI/NotI restriction fragment obtainable from the deposit containing clone AS268_{—}1 should be approximately 1000 bp.

The nucleotide sequence disclosed herein for AS268_{—}1 was searched against the GenBank and GeneSeq nucleotide sequence databases using BLASTN/BLASTX and FASTA search protocols. AS268_{—}1 demonstrated at least some similarity with sequences identified as M59743 (Rabbit cardiac muscle Ca-2+ release channel (ryanodine receptor) mRNA, complete cds) and X98330 (H. sapiens mRNA for ryanodine receptor 2). The predicted amino acid sequence disclosed herein for AS268_{—}1 was searched against the GenPept and GeneSeq amino acid sequence databases using the BLASTX search protocol. The predicted AS268_{—}1 protein demonstrated at least some similarity to sequences identified as X83933 (ryanodine receptor type 2 [Mus musculus]) and X98330 (ryanodine receptor 2 [Homo sapiens]). Based upon sequence similarity, AS268_{—}1 proteins and each similar protein or peptide may share at least some activity.

Clone “AS301_{—}2”

A polynucleotide of the present invention has been identified as clone “AS301_{—}2”. AS301_{—}2 was isolated from a human fetal brain cDNA library using methods which are selective for cDNAs encoding secreted proteins (see U.S. Pat. No. 5,536,637). AS301_{—}2 is a full-length clone, including the entire coding sequence of a secreted protein (also referred to herein as “AS301_{—}2 protein”).

The nucleotide sequence of AS301_{—}2 as presently determined is reported in SEQ ID NO:64, and includes a poly(A) tail. What applicants presently believe to be the proper reading frame and the predicted amino acid sequence of the AS301_{—}2 protein corresponding to the foregoing nucleotide sequence is reported in SEQ ID NO:65. Amino acids 26 to 38 of SEQ ID NO:65 are a possible leader/signal sequence, with the predicted mature amino acid sequence beginning at amino acid 39. Due to the hydrophobic nature of the predicted leader/signal sequence, it is likely to act as a transmembrane domain should the predicted leader/signal sequence not be separated from the remainder of the AS301_{—}2 protein.

The reverse complement of the AS301_{—}2 nucleotide sequence is reported in SEQ ID NO:311. The amino acid sequence of another protein that could be encoded by nucleotides 896 to 1189 of SEQ ID NO:311 is reported in SEQ ID NO:312.

The EcoRI/NotI restriction fragment obtainable from the deposit containing clone AS301_{—}2 should be approximately 2600 bp.

The nucleotide sequence disclosed herein for AS301_{—}2 was searched against the GenBank and GeneSeq nucleotide sequence databases using BLASTN/BLASTX and FASTA search protocols. AS301_{—}2 demonstrated at least some similarity with sequences identified as AA428294 (zw18c07.s1 Soares ovary tumor NbHOT Homo sapiens cDNA clone 769644 3′), AC000378 (Human Chromosome 11 pac pDJ11173a5, complete sequence), H16691 (ym26e09.r1 Homo sapiens cDNA clone 49167 5′ similar to contains PTR7 repetitive element), R83304 (yp82b08.s1 Homo sapiens cDNA clone 193911 3′), R83399 (yp82b08.r1 Homo sapiens cDNA clone 193911 5′), T98967 (ye66c02.r1 Homo sapiens cDNA clone 122690 5′), W41706 (mc65b04.r1 Soares mouse embryo NbME13.5 14.5 Mus musculus cDNA clone 353359 5′), and Z38485 (H. sapiens partial cDNA sequence; clone c-0dc09). The predicted amino acid sequence disclosed herein for AS301_{—}2 was searched against the GenPept and GeneSeq amino acid sequence databases using the BLASTX search protocol. The predicted AS301_{—}2 protein demonstrated at least some similarity to sequences identified as M27508 (beta-galactosidase related protein precursor [Homo sapiens]) and X56082 (endo-glucanase [Ruminococcus flavefaciens]). Based upon sequence similarity, AS301_{—}2 proteins and each similar protein or peptide may share at least some activity.

Clone “AU105_{—}14”

A polynucleotide of the present invention has been identified as clone “AU105_{—}14”. AU105_{—}14 was isolated from a human adult testes cDNA library using methods which are selective for cDNAs encoding secreted proteins (see U.S. Pat. No. 5,536,637). AU105_{—}14 is a full-length clone, including the entire coding sequence of a secreted protein (also referred to herein as “AU105_{—}14 protein”).

The nucleotide sequence of AU105_{—}14 as presently determined is reported in SEQ ID NO:66, and includes a poly(A) tail. What applicants presently believe to be the proper reading frame and the predicted amino acid sequence of the AU105_{—}14 protein corresponding to the foregoing nucleotide sequence is reported in SEQ ID NO:67. Amino acids 39 to 51 of SEQ ID NO:67 are a predicted leader/signal sequence, with the predicted mature amino acid sequence beginning at amino acid 52. Due to the hydrophobic nature of the predicted leader/signal sequence, it is likely to act as a transmembrane domain should the predicted leader/signal sequence not be separated from the remainder of the AU105_{—}14 protein.

The EcoRI/NotI restriction fragment obtainable from the deposit containing clone AU105_{—}14 should be approximately 2700 bp.

The nucleotide sequence disclosed herein for AU105_{—}14 was searched against the GenBank and GeneSeq nucleotide sequence databases using BLASTN/BLASTX and FASTA search protocols. AU105_{—}14 demonstrated at least some similarity with sequences identified as AA629020 (zu77a09.s1 Soares testis NHT Homo sapiens cDNAclone 743992 3′) and Z49235 (Human DNA sequence from cosmid L108f12, Huntington's Disease Region, chromosome 4p16.3). The predicted amino acid sequence disclosed herein for AU105_{—}14 was searched against the GenPept and GeneSeq amino acid sequence databases using the BLASTX search protocol. The predicted AU105_{—}14 protein demonstrated at least some similarity to sequences identified as Z77131 (C54C6.4 [Caenorhabditis elegans]). Based upon sequence similarity, AU105_{—}14 proteins and each similar protein or peptide may share at least some activity. The TopPredII computer program predicts three additional potential transmembrane domains within the AU105_{—}14 protein sequence, centered around amino acids 30, 120, and 160 of SEQ ID NO:67, respectively.

Clone “AU139_{—}2”

A polynucleotide of the present invention has been identified as clone “AU139_{—}2”. AU139_{—}2 was isolated from a human adult testes cDNA library using methods which are selective for cDNAs encoding secreted proteins (see U.S. Pat. No. 5,536,637). AU139_{—}2 is a full-length clone, including the entire coding sequence of a protein (also referred to herein as “AU139_{—}2 protein”).

The nucleotide sequence of AU139_{—}2 as presently determined is reported in SEQ ID NO:68, and includes a poly(A) tail. What applicants presently believe to be the proper reading frame and the predicted amino acid sequence of the AU139_{—}2 protein corresponding to the foregoing nucleotide sequence is reported in SEQ ID NO:69.

The amino acid sequence of another protein that could be encoded by nucleotides 422 to 796 of SEQ ID NO:68 is reported in SEQ ID NO:313. Amino acids 59 to 71 of SEQ ID NO:313 are a predicted leader/signal sequence, with the predicted mature amino acid sequence beginning at amino acid 72. Due to the hydrophobic nature of the predicted leader/signal sequence, it is likely to act as a transmembrane domain should the predicted leader/signal sequence not be separated from the remainder of the protein of SEQ ID NO:313.

The EcoRI/NotI restriction fragment obtainable from the deposit containing clone AU139_{—}2 should be approximately 1800 bp.

The nucleotide sequence disclosed herein for AU139_{—}2 was searched against the GenBank and GeneSeq nucleotide sequence databases using BLASTN/BLASTX and FASTA search protocols. AU139_{—}2 demonstrated at least some similarity with sequences identified as AA070392 (zm69g12.r1 Stratagene neuroepithelium (#937231) Homo sapiens cDNA clone 530950 5′ similar to SW YER9_YEAST P39966 PUTATIVE 51.4 KD PHOSPHATASE 2C IN ILV1-TRP2 INTERGENIC REGION; mRNA sequence), AA418416 (zv92g05.s1 Soares NhHMPu S1 Homo sapiens cDNA clone 767288 3′ similar to contains Alu repetitive element), H32905 (EST108441 Rattus sp. cDNA 5′ end), T06191 (EST04080 Homo sapiens cDNA clone HFBDQ07), T24369 (Human gene signature HUMGS06401), T30419 (EST16319 Homo sapiens cDNA 5′ end similar to None), U42383 (Mus musculus fibroblast growth factor inducible gene 13 (FIN13) mRNA, complete cds), U81159 (Bos taurus magnesium-dependent calcium inhibitable phosphatase MCPP mRNA, complete cds), and Y13936 (Homo sapiens mRNA for protein phosphatase 2C gamma). The predicted amino acid sequence disclosed herein for AU139_{—}2 was searched against the GenPept and GeneSeq amino acid sequence databases using the BLASTX search protocol. The predicted AU139_{—}2 protein demonstrated at least some similarity to sequences identified as L34881 (protein phosphatase 2C (ptc2+) [Schizosaccharomyces pombe]), U42383 (serine/threonine type 2C phosphatase homolog; Method conceptual translation supplied by author [Mus musculus]), U81159 (magnesium-dependent calcium inhibitable phosphatase [Bos taurus]), Y13936 (protein phosphatase 2C gamma [Homo sapiens]), and Z35817 (ORF YBL056w [Saccharomyces cerevisiae]). Based upon sequence similarity, AU139_{—}2 proteins and each similar protein or peptide may share at least some activity.

Clone “AZ302_{—}1”

A polynucleotide of the present invention has been identified as clone “AZ302_{—}1”. AZ302_{—}1 was isolated from a human adult colon (adenocarcinoma Caco2 cell line) cDNA library using methods which are selective for cDNAs encoding secreted proteins (see U.S. Pat. No. 5,536,637). AZ302_{—}1 is a full-length clone, including the entire coding sequence of a secreted protein (also referred to herein as “A7302_{—}1 protein”).

The nucleotide sequence of Az302_{—}1 as presently determined is reported in SEQ ID NO:70, and includes a poly(A) tail. What applicants presently believe to be the proper reading frame and the predicted amino acid sequence of the AZ302_{—}1 protein corresponding to the foregoing nucleotide sequence is reported in SEQ ID NO:71. Amino acids 163 to 175 of SEQ ID NO:71 are a possible leader/signal sequence, with the predicted mature amino acid sequence beginning at amino acid 176. Due to the hydrophobic nature of the predicted leader/signal sequence, it is likely to act as a transmembrane domain should the predicted leader/signal sequence not be separated from the remainder of the AZ302_{—}1 protein.

The EcoRI/NotI restriction fragment obtainable from the deposit containing clone AZ302_{—}1 should be approximately 1400 bp.

The nucleotide sequence disclosed herein for AZ302_{—}1 was searched against the GenBank and GeneSeq nucleotide sequence databases using BLASTN/BLASTX and FASTA search protocols. AZ302_{—}1 demonstrated at least some similarity with sequences identified as AA046898 (zf47c09.r1 Soares retina N2b4HR Homo sapiens cDNA clone 380080 5′), AA065625 (mm42d10.r1 Stratagene mouse melanoma (#937312) Mus musculus cDNA clone 524179 5′), AA068828 (mm59c09.r1 Stratagene mouse embryonic carcinoma (#937317) Mus musculus cDNA clone 532720 5′), AA151630 (zo29g02.r1 Stratagene colon (#937204) Homo sapiens cDNA clone 588338 5′), AA152019 (zl49c12.r1 Soares pregnant uterus NbHPU Homo sapiens cDNA clone 505270 5′), AA163971 (ms40a01.r1 Life Tech mouse embryo 13 5dpc 10666014 Mus musculus cDNA clone 613992 5′), R02197 (ye83a03.r1 Homo sapiens cDNA clone 124300 5′), T24189 (Human gene signature HUMGS06196), W22546 (69C1 Human retina cDNA Tsp509I-cleaved sublibrary Homo sapiens cDNA not directional), and Z36313 (M. musculus mRNA (clone 208) for expressed sequence tag (EST)). The predicted amino acid sequence disclosed herein for AZ302_{—}1 was searched against the GenPept and GeneSeq amino acid sequence databases using the BLASTX search protocol. The predicted AZ302_{—}1 protein demonstrated at least some similarity to sequences identified as U53225 (sorting nexin 1 [Homo sapiens]) and Z49311 (ORF YJL036w [Saccharomyces cerevisiae]). Based upon sequence similarity, AZ302_{—}1 proteins and each similar protein or peptide may share at least some activity.

AZ302_{—}1 protein was expressed in a COS cell expression system, and an expressed protein band of approximately 49 kDa was detected in membrane fractions using SDS polyacrylamide gel electrophoresis.

Clone “D147_{—}17”

A polynucleotide of the present invention has been identified as clone “D147_{—}17”. A cDNA clone was first isolated from a human adult blood (peripheral blood mononuclear cells activated by treatment with concanavalin A and phorbol myristate acetate) cDNA library using methods which are selective for cDNAs encoding secreted proteins (see U.S. Pat. No. 5,536,637). This cDNA clone was then used to isolate D147_{—}17 from a human adult blood (peripheral blood mononuclear cells activated by treatment with phytohemagglutinin, phorbol myristate acetate, and mixed lymphocyte reaction) cDNA library. D147_{—}17 is a full-length clone, including the entire coding sequence of a secreted protein (also referred to herein as “D147_{—}17 protein”).

The nucleotide sequence of D147_{—}17 as presently determined is reported in SEQ ID NO:72, and includes a poly(A) tail. What applicants presently believe to be the proper reading frame and the predicted amino acid sequence of the D147_{—}17 protein corresponding to the foregoing nucleotide sequence is reported in SEQ ID NO:73. Amino acids 219 to 231 of SEQ ID NO:73 are a predicted leader/signal sequence, with the predicted mature amino acid sequence beginning at amino acid 232. Due to the hydrophobic nature of the predicted leader/signal sequence, it is likely to act as a transmembrane domain should the predicted leader/signal sequence not be separated from the remainder of the D147_{—}17 protein.

The EcoRI/NotI restriction fragment obtainable from the deposit containing clone D147_{—}17 should be approximately 2500 bp.

The nucleotide sequence disclosed herein for D147_{—}17 was searched against the GenBank and GeneSeq nucleotide sequence databases using BLASTN/BLASTX and FASTA search protocols. D147_{—}17 demonstrated at least some similarity with sequences identified as AA011178 (ze22c01.s1 Soares fetal heart NbHH19W Homo sapiens cDNA clone 359712 3′), AA044195 (zk50e05.r1 Soares pregnant uterus NbHPU Homo sapiens cDNA clone 486272 5′), AA181797 (zp55g05.r1 Stratagene NT2 neuronal precursor 937230 Homo sapiens cDNA clone 613400 5′ similar to WP C09G4.1 CE03978), H14129 (ym65b04.r1 Homo sapiens cDNA clone 163759 5′), T21374 (Human gene signature HUMGS02738), and T24634 (Human gene signature HUMGS06694). The predicted amino acid sequence disclosed herein for D147_{—}17 was searched against the GenPept and GeneSeq amino acid sequence databases using the BLASTX search protocol. The predicted D147_{—}17 protein demonstrated at least some similarity to sequences identified as R20230 (hUOG-1), R86811 (Saccharomyces cerevisiae mutant LAG1 protein), and U42438 (similar to S. cerevisiae longevity-assurance protein 1 (SP P38703) [Caenorhabditis elegans]). Based upon sequence similarity, D147_{—}17 proteins and each similar protein or peptide may share at least some activity. D147_{—}17 nucleotide and protein sequences also demonstrate at least some similarity to those of murine clone K39_{—}7 described below. The TopPredII computer program predicts six potential transmembrane domains within the D147_{—}17 protein sequence, centered around amino acids 50, 140, 180, 220, 280, and 300 of SEQ ID NO:73, respectively. The nucleotide sequence of D147_{—}17 indicates that it may contain one or more GCCC repeats.

D147_{—}17 protein was expressed in a COS cell expression system, and an expressed protein band of approximately 40 kDa was detected in membrane fractions using SDS polyacrylamide gel electrophoresis.

Clone “O75_{—}9”

A polynucleotide of the present invention has been identified as clone “O75_{—}9”. O75_{—}9 was isolated from a human adult blood (dendritic cells) cDNA library using methods which are selective for cDNAs encoding secreted proteins (see U.S. Pat. No. 5,536,637). O75_{—}9 is a full-length clone, including the entire coding sequence of a secreted protein (also referred to herein as “O75_{—}9 protein”).

The nucleotide sequence of the 5′ portion of O75_{—}9 as presently determined is reported in SEQ ID NO:74. What applicants presently believe is the proper reading frame for the coding region is indicated in SEQ ID NO:75. The predicted amino acid sequence of the O75_{—}9 protein corresponding to the foregoing nucleotide sequence is reported in SEQ ID NO:75. Amino acids 4 to 16 of SEQ ID NO:75 are a predicted leader/signal sequence, with the predicted mature amino acid sequence beginning at amino acid 17. Due to the hydrophobic nature of the predicted leader/signal sequence, it is likely to act as a transmembrane domain should the predicted leader/signal sequence not be separated from the remainder of the O75_{—}9 protein. Additional nucleotide sequence from the 3′ portion of O75_{—}9 is reported in SEQ ID NO:76.

The EcoRI/NotI restriction fragment obtainable from the deposit containing clone O75_{—}9 should be approximately 1450 bp.

The nucleotide sequence disclosed herein for O75_{—}9 was searched against the GenBank and GeneSeq nucleotide sequence databases using BLASTN/BLASTX and FASTA search protocols. O75_{—}9 demonstrated at least some similarity with sequences identified as T31720 (EST37588 Homo sapiens cDNA 5′ end similar to None). Based upon sequence similarity, O75_{—}9 proteins and each similar protein or peptide may share at least some activity.

Isolates “AS152_{—}1” and “AS152_{—}2” of Clone AS152

Polynucleotides of the present invention has been identified as clone “AS152_{—}1”. and clone “AS152_{—}2”. AS152_{—}1 and AS152_{—}2 were isolated from a human fetal brain cDNA library using methods which are selective for cDNAs encoding secreted proteins (see U.S. Pat. No. 5,536,637). AS152_{—}1 and AS152_{—}2 are a full-length clones, including the entire coding sequence of a secreted protein (also referred to herein as “AS152 protein”).

The nucleotide sequence of the 5′ portion of AS152_{—}1 as presently determined is reported in SEQ ID NO:77. What applicants presently believe is the proper reading frame for the coding region is indicated in SEQ ID NO:78. The predicted amino acid sequence of the AS152_{—}1 protein corresponding to the foregoing nucleotide sequence is reported in SEQ ID NO:78. Amino acids 10 to 22 of SEQ ID NO:78 are a predicted leader/signal sequence, with the predicted mature amino acid sequence beginning at amino acid 23. Due to the hydrophobic nature of the predicted leader/signal sequence, it is likely to act as a transmembrane domain should the predicted leader/signal sequence not be separated from the remainder of the AS152_{—}1 protein. Additional nucleotide sequence from the 3′ portion of AS152_{—}1, including a poly(A) tail, is reported in SEQ ID NO:79.

The nucleotide sequence of the 5′ portion of AS152_{—}2 as presently determined is reported in SEQ ID NO:80. What applicants presently believe is the proper reading frame for the coding region is indicated in SEQ ID NO:81. The predicted amino acid sequence of the AS152_{—}2 protein corresponding to the foregoing nucleotide sequence is reported in SEQ ID NO:81. Amino acids 10 to 22 of SEQ ID NO:81 are a predicted leader/signal sequence, with the predicted mature amino acid sequence beginning at amino acid 23. Due to the hydrophobic nature of the predicted leader/signal sequence, it is likely to act as a transmembrane domain should the predicted leader/signal sequence not be separated from the remainder of the AS152_{—}2 protein. Additional nucleotide sequence from the 3′ portion of AS152_{—}2, including a poly(A) tail, is reported in SEQ ID NO:82.

A nucleotide sequence derived from the AS152_{—}1 and AS152_{—}2 nucleotide sequnces (SEQ ID NO:77 and SEQ ID NO:80) is reported in SEQ ID NO:314. The amino acid sequence of another protein that could be encoded by nucleotides 800 to 892 of SEQ ID NO:77 is reported in SEQ ID NO:315. The amino acid sequence of another protein that could be encoded by nucleotides 220 to 282 of SEQ ID NO:80, when presented in the reverse orientation, is reported in SEQ ID NO:316.

The EcoRI/NotI restriction fragment obtainable from the deposit containing clone AS152_{—}1 should be approximately 2350 bp, and the EcoRI/NotI restriction fragment obtainable from the deposit containing clone AS152_{—}2 should be approximately 2000 bp.

The nucleotide sequence disclosed herein for AS152_{—}1 was searched against the GenBank and GeneSeq nucleotide sequence databases using BLASTN/BLASTX and FASTA search protocols. AS152_{—}1 demonstrated at least some similarity with sequences identified as H96739 (yw08h12.s1 Homo sapiens cDNA clone 251687 3′), W69601 (zd47d05.r1 Soares fetal heart NbHH19W Homo sapiens cDNA clone 343785 5′), and Z29930 (H. sapiens partial cDNA sequence; clone HEA28A; single read). Based upon sequence similarity, AS152_{—}1 proteins and each similar protein or peptide may share at least some activity.

The nucleotide sequence disclosed herein for AS152_{—}2 was searched against the GenBank and GeneSeq nucleotide sequence databases using BLASTN/BLASTX and FASTA search protocols. AS152_{—}2 demonstrated at least some similarity with sequences identified as H29446 (ym32b10.s1 Homo sapiens cDNA clone 49741 3′), N44673 (yy22a05.r1 Homo sapiens cDNA clone 271952 5′), W69601 (zd47d05.r1 Soares fetal heart NbHH19W Homo sapiens cDNA clone 343785 5′), W73977 (zd71h09.s1 Soares fetal heart NbHH19W Homo sapiens cDNA clone 346145 3′), and Z29930 (H. sapiens partial cDNA sequence; clone HEA28A; single read). Based upon sequence similarity, AS152_{—}2 proteins and each similar protein or peptide may share at least some activity.

Clone “AS167_{—}3”

A polynucleotide of the present invention has been identified as clone “AS167_{—}3”. AS167_{—}3 was isolated from a human fetal brain cDNA library using methods which are selective for cDNAs encoding secreted proteins (see U.S. Pat. No. 5,536,637). AS167_{—}3 is a full-length clone, including the entire coding sequence of a secreted protein (also referred to herein as “AS167_{—}3 protein”).

The nucleotide sequence of AS167_{—}3 as presently determined is reported in SEQ ID NO:83, and includes a poly(A) tail. What applicants presently believe to be the proper reading frame and the predicted amino acid sequence of the AS167_{—}3 protein corresponding to the foregoing nucleotide sequence is reported in SEQ ID NO:84. Amino acids 8 to 20 of SEQ ID NO:84 are a predicted leader/signal sequence, with the predicted mature amino acid sequence beginning at amino acid 21. Due to the hydrophobic nature of the predicted leader/signal sequence, it is likely to act as a transmembrane domain should the predicted leader/signal sequence not be separated from the remainder of the AS167_{—}3 protein.

The EcoRI/NotI restriction fragment obtainable from the deposit containing clone AS167_{—}3 should be approximately 2300 bp.

The nucleotide sequence disclosed herein for AS167_{—}3 was searched against the GenBank and GeneSeq nucleotide sequence databases using BLASTN/BLASTX and FASTA search protocols. AS167_{—}3 demonstrated at least some similarity with sequences identified as AA282911 (zt14c11.s1 NCI_CGAP_GCB1 Homo sapiens cDNA clone IMAGE:713108 3′, mRNA sequence), AB002342 (Human mRNA for KIAA0344 gene, complete cds), H89618 (yu81a04.r1 Homo sapiens cDNA clone 240174 5′), and T90556 (yd40g01.s1 Homo sapiens cDNA clone 110736 3′). The predicted amino acid sequence disclosed herein for AS167_{—}3 was searched against the GenPept and GeneSeq amino acid sequence databases using the BLASTX search protocol. The predicted AS167_{—}3 protein demonstrated at least some similarity to sequences identified as AB002342 (KIAA0344 [Homo sapiens]) and X16365 (SFL2 gene poduct (AA 1-669) [Saccharomyces cerevisiae]). Based upon sequence similarity, AS167_{—}3 proteins and each similar protein or peptide may share at least some activity. The TopPredII computer program predicts three potential transmembrane domains within the AS167_{—}3 protein sequence, centered around amino acids 18, 456, and 528 of SEQ ID NO:84, respectively, and three additional putative transmembrane domains.

AS167_{—}3 protein was expressed in a COS cell expression system, and an expressed protein band of approximately 30 kDa was detected in conditioned medium using SDS polyacrylamide gel electrophoresis.

Clone “AU47_{—}8”

A polynucleotide of the present invention has been identified as clone “AU47_{—}8”. AU47_{—}8 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AU47_{—}8 is a full-length clone, including the entire coding sequence of a secreted protein (also referred to herein as “AU47_{—}8 protein”).

The nucleotide sequence of AU47_{—}8 as presently determined is reported in SEQ ID NO:85, and includes a poly(A) tail. What applicants presently believe to be the proper reading frame and the predicted amino acid sequence of the AU47_{—}8 protein corresponding to the foregoing nucleotide sequence is reported in SEQ ID NO:86. Amino acids 5 to 17 of SEQ ID NO:86 are a predicted leader/signal sequence, with the predicted mature amino acid sequence beginning at amino acid 18. Due to the hydrophobic nature of the predicted leader/signal sequence, it is likely to act as a transmembrane domain should the predicted leader/signal sequence not be separated from the remainder of the AU47_{—}8 protein.

The EcoRI/NotI restriction fragment obtainable from the deposit containing clone AU47_{—}8 should be approximately 4200 bp.

The nucleotide sequence disclosed herein for AU47_{—}8 was searched against the GenBank and GeneSeq nucleotide sequence databases using BLASTN/BLASTX and FASTA search protocols. AU47_{—}8 demonstrated at least some similarity with sequences identified as AA397955 (zt79h12.r1 Soares testis NHT Homo sapiens cDNA clone 728615 5′, mRNA sequence), AA416585 (zu05f02.s1 Soares testis NHT Homo sapiens cDNA clone 730971 3′), AC003669 (Homo sapiens Xp22 BAC GS-594A7 (Genome Systems Human BAC library) contains Bmx gene, complete sequence), M55333 (Mouse testis-specific angiotensin-converting enzyme mRNA, complete cds), Q04027 (Human angiotensin converting enzyme (ACE) gene), and U03708 (Rattus norvegicus Heidelberg angiotensin converting enzyme (ACE) mRNA, complete cds). The predicted amino acid sequence disclosed herein for AU47_{—}8 was searched against the GenPept and GeneSeq amino acid sequence databases using the BLASTX search protocol. The predicted AU47_{—}8 protein demonstrated at least some similarity to sequences identified as L40175 (angiotensin converting enzyme [Gallus gallus]), M55333 (angiotensin converting enzyme [Mus musculus]), M58579 (angiotensin converting enzyme [Oryctolagus cuniculus]), R10426 (Human testicular angiotensin conversion enzyme), and U03708 (angiotensin converting enzyme [Rattus norvegicus]). Based upon sequence similarity, AU47_{—}8 proteins and each similar protein or peptide may share at least some activity. AU47_{—}8 appears to encode an angiotensin-converting enzyme gene family member. Angiotensin-converting enzyme (ACE) is a zinc-containing dipeptidyl carboxypeptidase that catalyzes the conversion of angiotensin I to the potent vasoconstrictor angiotensin II. In keeping with this function, AU47_{—}8, like the other ACEs, contains the neutral zinc metallopeptidases, zinc-binding region signature (around residue 376 of SEQ ID NO:86). ACEs range in size (species to species) from roughly 630 to 1300 amino acids, so the size of the predicted AU47_{—}8 full-length protein (805 amino acids), while shorter than the Oryctolagus cuniculus ACE (1310 amino acids), is still in a size range consistant for an ACE. The TopPredII computer program predicts three additional potential transmembrane domains within the AU47_{—}8 protein sequence, centered around amino acids 260, 446, and 740 of SEQ ID NO:86, respectively.

AU47_{—}8 protein was expressed in a COS cell expression system, and an expressed protein band of approximately 100 kDa was detected in conditioned medium and membrane fractions using SDS polyacrylamide gel electrophoresis.

Clone “AU122_{—}1”

A polynucleotide of the present invention has been identified as clone “AU122_{—}1”. AU122_{—}1 was isolated from a human adult testes cDNA library using methods which are selective for cDNAs encoding secreted proteins (see U.S. Pat. No. 5,536,637). AU122_{—}1 is a full-length clone, including the entire coding sequence of a secreted protein (also referred to herein as “AU122_{—}1 protein”).

The nucleotide sequence of AU122_{—}1 as presently determined is reported in SEQ ID NO:87, and includes a poly(A) tail. What applicants presently believe to be the proper reading frame and the predicted amino acid sequence of the AU122_{—}1 protein corresponding to the foregoing nucleotide sequence is reported in SEQ ID NO:88. Amino acids 55 to 67 of SEQ ID NO:88 are a possible leader/signal sequence, with the predicted mature amino add sequence beginning at amino acid 68. Due to the hydrophobic nature of this possible leader/signal sequence, it is likely to act as a transmembrane domain should it not be separated from the remainder of the AU122_{—}1 protein.

The EcoRI/NotI restriction fragment obtainable from the deposit containing clone AU122_{—}1 should be approximately 1500 bp.

The nucleotide sequence disclosed herein for AU122_{—}1 was searched against the GenBank and GeneSeq nucleotide sequence databases using BLASTN/BLASTX and FASTA search protocols. AU122_{—}1 demonstrated at least some similarity with sequences identified as AA757386 (AA757386 ah98c06.s1 Soares NFL T GBC S1 Homo sapiens cDNA clone 1327114 3′) and U46493 (Cloning vector pFlp recombinase gene, complete cds). Based upon sequence similarity, AU122_{—}1 proteins and each similar protein or peptide may share at least some activity. The TopPredII computer program predicts an additional potential transmembrane domain within the AU122_{—}1 protein sequence centered around amino acid 50 of SEQ ID NO:88.

AU122_{—}1 protein was expressed in a COS cell expression system, and an expressed protein band of approximately 90 kDa was detected in conditioned medium using SDS polyacrylamide gel electrophoresis.

Clone “BF208_{—}1”

A polynucleotide of the present invention has been identified as clone “BF208_{—}1”. BF208_{—}1 was isolated from a human fetal brain cDNA library using methods which are selective for cDNAs encoding secreted proteins (see U.S. Pat. No. 5,536,637). BF208_{—}1 is a full-length clone, including the entire coding sequence of a protein (also referred to herein as “BF208_{—}1 protein”).

The nucleotide sequence of the 5′ portion of BF208_{—}1 as presently determined is reported in SEQ ID NO:89. What applicants presently believe is the proper reading frame for the coding region is indicated in SEQ ID NO:90. The predicted amino acid sequence of the BF208_{—}1 protein corresponding to the foregoing nucleotide sequence is reported in SEQ ID NO:90. Additional nucleotide sequence from the 3′ portion of BF208_{—}1, including a poly(A) tail, is reported in SEQ ID NO:91.

The EcoRI/NotI restriction fragment obtainable from the deposit containing clone BF208_{—}1 should be approximately 2700 bp.

The nucleotide sequence disclosed herein for BF208_{—}1 was searched against the GenBank and GeneSeq nucleotide sequence databases using BLASTN/BLASTX and FASTA search protocols. BF208_{—}1 demonstrated at least some similarity with sequences identified as D79956 (Human aorta cDNA 5′-end GEN-363D02) and D83032 (Human mRNA for nuclear protein, NP220, complete cds). The predicted amino acid sequence disclosed herein for BF208_{—}1 was searched against the GenPept and GeneSeq amino acid sequence databases using the BLASTX search protocol. The predicted BF208_{—}1 protein demonstrated at least some similarity to sequences identified as D83032 (nuclear protein, NP220 [Homo sapiens]). Based upon sequence similarity, BF208_{—}1 proteins and each similar protein or peptide may share at least some activity.

Clone “BG513_{—}19”

A polynucleotide of the present invention has been identified as clone “BG513_{—}19”. BG513_{—}19 was isolated from a human adult brain cDNA library using methods which are selective for cDNAs encoding secreted proteins (see U.S. Pat. No. 5,536,637). BG513_{—}19 is a full-length clone, including the entire coding sequence of a secreted protein (also referred to herein as “BG513_{—}19 protein”).

The nucleotide sequence of BG513_{—}19 as presently determined is reported in SEQ ID NO:92, and includes a poly(A) tail. What applicants presently believe to be the proper reading frame and the predicted amino acid sequence of the BG513_{—}19 protein corresponding to the foregoing nucleotide sequence is reported in SEQ ID NO:93.

The EcoRI/NotI restriction fragment obtainable from the deposit containing clone BG513_{—}19 should be approximately 3000 bp.

The nucleotide sequence disclosed herein for BG513_{—}19 was searched against the GenBank and GeneSeq nucleotide sequence databases using BLASTN/BLASTX and FASTA search protocols. BG513_{—}19 demonstrated at least some similarity with sequences identified as B79106 (CIT-HSP-691J23.TV CIT-HSP Homo sapiens genomic clone 691J23). Based upon sequence similarity, BG513_{—}19 proteins and each similar protein or peptide may share at least some activity. The TopPredII computer program predicts a potential transmembrane domain near the C-terminal end of the BG513_{—}19 protein sequence, centered around amino acid 70 of SEQ ID NO:93. The nucleotide sequence of BG513_{—}19 indicates that it may contain one or more of the following repetitive elements: MLT, LTR.

BG513_{—}19 protein was expressed in a COS cell expression system, and an expressed protein band of approximately 30 kDa was detected in conditioned medium using SDS polyacrylamide gel electrophoresis.

Clone “BG556_{—}8”

A polynucleotide of the present invention has been identified as clone “BG556_{—}8”. BG556_{—}8 was isolated from a human adult brain cDNA library using methods which are selective for cDNAs encoding secreted proteins (see U.S. Pat. No. 5,536,637). BG556_{—}8 is a full-length clone, including the entire coding sequence of a secreted protein (also referred to herein as “BG556_{—}8 protein”).

The partial nucleotide sequence of BG556_{—}8, including its 3′ end as presently determined is reported in SEQ ID NO:95. What applicants presently believe is the proper reading frame for the coding region is indicated in SEQ ID NO:96. The predicted amino acid sequence of the BG556_{—}8 protein corresponding to the foregoing nucleotide sequence is reported in SEQ ID NO:96. Amino acids 27 to 39 of SEQ ID NO:96 are a predicted leader/signal sequence, with the predicted mature amino acid sequence beginning at amino acid 40. Due to the hydrophobic nature of the predicted leader/signal sequence, it is likely to act as a transmembrane domain should the predicted leader/signal sequence not be separated from the remainder of the BG556_{—}8 protein. Additional nucleotide sequence from the 5′ portion of BG556_{—}8 is reported in SEQ ID NO:94.

The EcoRI/NotI restriction fragment obtainable from the deposit containing clone BG556_{—}8 should be approximately 1700 bp.

The nucleotide sequence disclosed herein for BG556_{—}8 was searched against the GenBank and GeneSeq nucleotide sequence databases using BLASTN/BLASTX and FASTA search protocols. BG556_{—}8 demonstrated at least some similarity with sequences identified as L00837 (Human chromosome 4 sequence-tagged site STS4-396). Based upon sequence similarity, BG556_{—}8 proteins and each similar protein or peptide may share at least some activity.

Isolates “C195_{—}1” and “C195_{—}4” of Clone C195

Polynucleotides of the present invention have been identified as clone “C195_{—}1” and clone “C195_{—}1”. A cDNA clone was first isolated from a human adult blood (peripheral blood mononuclear cells activated by treatment with concanavalin A and phorbol myristate acetate) cDNA library using methods which are selective for cDNAs encoding secreted proteins (see U.S. Pat. No. 5,536,637). This cDNA clone was then used to isolate C195_{—}1 and C195_{—}4 from a human adult blood (peripheral blood mononuclear cells activated by treatment with phytohemagglutinin, phorbol myristate acetate, and mixed lymphocyte reaction) cDNA library. C195_{—}1 and C195_{—}4 are full-length clones, each including the entire coding sequence of a protein (also referred to herein as “C195_{—}1 protein” or “C195_{—}4 protein”).

The nucleotide sequence of the 5′ portion of C195_{—}1 as presently determined is reported in SEQ ID NO:97. What applicants presently believe is the proper reading frame for the coding region is indicated in SEQ ID NO:98. The predicted amino acid sequence of the C195_{—}1 protein corresponding to the foregoing nucleotide sequence is reported in SEQ ID NO:98. Additional nucleotide sequence from the 3′ portion of C195_{—}1, including a poly(A) tail, is reported in SEQ ID NO:99.

Additional nucleotide sequence information for clone C195_{—}1 is presented in SEQ ID NO:317. The amino acid sequence of a protein that could be encoded by nucleotides 14 to 799 of SEQ ID NO:317 is reported in SEQ ID NO:318.

The nucleotide sequence of the 5′ portion of C195_{—}4 as presently determined is reported in SEQ ID NO:100. An additional internal nucleotide sequence from C195_{—}4 as presently determined is reported in SEQ ID NO:101. What applicants believe is the proper reading frame and the predicted amino acid sequence encoded by such internal sequence is reported in SEQ ID NO:102. Additional nucleotide sequence from the 3′ portion of C195_{—}4, including a poly(A) tail, is reported in SEQ ID NO:103.

The EcoRI/NotI restriction fragment obtainable from the deposit containing clone C195_{—}1 should be approximately 1350 bp, and the EcoRI/NotI restriction fragment obtainable from the deposit containing clone C195_{—}4 should be approximately 1700 bp.

The nucleotide sequences disclosed herein for C195_{—}1 and C195_{—4} were searched against the GenBank and GeneSeq nucleotide sequence databases using BLASTN/BLASTX and FASTA search protocols. C195_{—}1 demonstrated at least some similarity with sequences identified as H11976 (ym15f12.r1 Homo sapiens cDNA clone 48025 5′), R17211 (yg11g06.r1 Homo sapiens cDNA clone 31937 5′), and X96586 (H. sapiens mRNA for FAN protein). C195_{—}4 demonstrated at least some similarity with sequences identified as N54820 (yv31f05.s1 Homo sapiens cDNA clone 244353 3′), R17211 (yg11g06.r1 Homo sapiens cDNA clone 31937 5′), and X96586 (H. sapiens mRNA for FAN protein). The predicted amino acid sequences disclosed herein for C195_{—}1 and C195_{—}4 were searched against the GenPept and GeneSeq amino acid sequence databases using the BLASTX search protocol. The predicted C195_{—}1 protein and the predicted C195_{—}4 protein each demonstrated at least some similarity to sequences identified as U27569 (LACK [Leishmania chagasi]) and X96586 (FAN protein [Homo sapiens]). FAN protein, which contains WD domains, couples the p55 TNF receptor to neutral sphingomyelinase.

Based upon sequence similarity, C195_{—}1 proteins, C195_{—}4 proteins, and each similar protein or peptide may share at least some activity.

Clone “O276_{—}16”

A polynucleotide of the present invention has been identified as clone “O276_{—}16”. O276_{—}16 was isolated from a human adult blood (dendritic cells) cDNA library using methods which are selective for cDNAs encoding secreted proteins (see U.S. Pat. No. 5,536,637). O276_{—}16 is a full-length clone, including the entire coding sequence of a secreted protein (also referred to herein as “O276_{—}16 protein”).

The nucleotide sequence of O276_{—}16 as presently determined is reported in SEQ ID NO:104. What applicants presently believe to be the proper reading frame and the predicted amino acid sequence of the O276_{—}16 protein corresponding to the foregoing nucleotide sequence is reported in SEQ ID NO:105. The amino acid sequence of another protein that could be encoded by nucleotides 561 to 710 of SEQ ID NO:104 is reported in SEQ ID NO:319.

The EcoRI/NotI restriction fragment obtainable from the deposit containing clone O276_{—}16 should be approximately 700 bp.

The nucleotide sequence disclosed herein for O276_{—}16 was searched against the GenBank and GeneSeq nucleotide sequence databases using BLASTN/BLASTX and FASTA search protocols. O276_{—}16 demonstrated at least some similarity with sequences identified as U39779 (beta-galactosidase alpha polypeptide [Cloning vector pTriplEx]). Based upon sequence similarity, O276_{—}16 proteins and each similar protein or peptide may share at least some activity.

Clone “AC41_{—}1”

A polynucleotide of the present invention has been identified as clone “AC41_{—}1”. AC41_{—}1 was isolated from a human adult placenta cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AC41_{—}1 is a full-length clone, including the entire coding sequence of a secreted protein (also referred to herein as “AC41_{—}1 protein”).

The nucleotide sequence of AC41_{—}1 as presently determined is reported in SEQ ID NO:106, and includes a poly(A) tail. What applicants presently believe to be the proper reading frame and the predicted amino acid sequence of the AC41_{—}1 protein corresponding to the foregoing nucleotide sequence is reported in SEQ ID NO:107. Amino acids 10 to 22 of SEQ ID NO:107 are a predicted leader/signal sequence, with the predicted mature amino acid sequence beginning at amino acid 23. Due to the hydrophobic nature of the predicted leader/signal sequence, it is likely to act as a transmembrane domain should the predicted leader/signal sequence not be separated from the remainder of the AC41_{—}1 protein.

The EcoRI/NotI restriction fragment obtainable from the deposit containing clone AC41_{—}1 should be approximately 1000 bp.

The nucleotide sequence disclosed herein for AC41_{—}1 was searched against the GenBank and GeneSeq nucleotide sequence databases using BLASTN/BLASTX and FASTA search protocols. AC41_{—}1 demonstrated at least some similarity with sequences identified as L20319 (Rattus norvegicus developmentally regulated protein mRNA, complete cds), U46493 (Cloning vector pFlp recombinase gene, complete cds), and Z22650 (H. sapiens insertion polymorphism DNA). The predicted amino acid sequence disclosed herein for AC41_{—}1 was searched against the GenPept and GeneSeq amino acid sequence databases using the BLASTX search protocol. The predicted AC41_{—}1 protein demonstrated at least some similarity with sequences identified as L20319 (developmentally regulated protein [Rattus norvegicus]) and X12544 (3 HLA-DR B protein precursor (AA −29 to 267) [Homo sapiens]). Based upon sequence similarity, AC41_{—}1 proteins and each similar protein or peptide may share at least some activity.

AC41_{—}1 protein was expressed in a COS cell expression system, and an expressed protein band of approximately 20 kDa was detected in conditioned medium and membrane fractions using SDS polyacrylamide gel electrophoresis.

Clone “AC222_{—}1”

A polynucleotide of the present invention has been identified as clone “AC222_{—}1”. AC222_{—}1 was isolated from a human adult placenta cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AC222_{—}1 is a full-length clone, including the entire coding sequence of a secreted protein (also referred to herein as “AC222_{—}1 protein”).

The nucleotide sequence of AC222_{—}1 as presently determined is reported in SEQ ID NO:108, and includes a poly(A) tail. What applicants presently believe to be the proper reading frame and the predicted amino acid sequence of the AC222_{—}1 protein corresponding to the foregoing nucleotide sequence is reported in SEQ ID NO:109. Amino acids 7 to 19 of SEQ ID NO:109 are a predicted leader/signal sequence, with the predicted mature amino acid sequence beginning at amino acid 20. Due to the hydrophobic nature of the predicted leader/signal sequence, it is likely to act as a transmembrane domain should the predicted leader/signal sequence not be separated from the remainder of the AC222_{—}1 protein.

The EcoRI/NotI restriction fragment obtainable from the deposit containing clone AC222_{—}1 should be approximately 1400 bp.

The nucleotide sequence disclosed herein for AC222_{—}1 was searched against the GenBank and GeneSeq nucleotide sequence databases using BLASTN/BLASTX and FASTA search protocols. AC222_{—}1 demonstrated at least some similarity with sequences identified as D10485 (Chicken mRNA for proteoglycan (PG-Lb) core protein, complete cds), D78274 (Mouse mRNA for proteoglycan, complete cds), N22463 (yw34c10.s1 Homo sapiens cDNA clone 254130 3′), U59111 (Human dermatan sulfate proteoglycan 3 (DSPG3) mRNA, complete cds), U77127 (Bos taurus epiphycan mRNA, complete cds), and Z32693 (E. coli pT7hGH_pl DNA, 6160 bp). The predicted amino acid sequence disclosed herein for AC222_{—}1 was searched against the GenPept and GeneSeq amino acid sequence databases using the BLASTX search protocol. The predicted AC222_{—}1 protein demonstrated at least some similarity with sequences identified as D10485 (proteoglycan core protein [Gallus gallus]), D78274 (proteoglycan [Mus musculus]), U77127 (epiphycan [Bos taurus]), and U59111 (dermatan sulfate proteoglycan 3 [Homo sapiens]). Based upon sequence similarity, AC222_{—}1 proteins and each similar protein or peptide may share at least some activity.

AC222_{—}1 protein was expressed in a COS cell expression system, and an expressed protein band of approximately 58 kDa was detected in conditioned medium using SDS polyacrylamide gel electrophoresis.

Clone “AJ143_{—}1”

A polynucleotide of the present invention has been identified as clone “AJ143_{—}1”. AJ143_{—}1 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AJ143_{—}1 is a full-length clone, including the entire coding sequence of a secreted protein (also referred to herein as “AJ143_{—}1 protein”).

The nucleotide sequence of AJ143_{—}1 as presently determined is reported in SEQ ID NO:110, and includes a poly(A) tail. What applicants presently believe to be the proper reading frame and the predicted amino acid sequence of the AJ143_{—}1 protein corresponding to the foregoing nucleotide sequence is reported in SEQ ID NO:111. Amino acids 2 to 14 of SEQ ID NO:111 are a predicted leader/signal sequence, with the predicted mature amino acid sequence beginning at amino acid 15. Due to the hydrophobic nature of the predicted leader/signal sequence, it is likely to act as a transmembrane domain should the predicted leader/signal sequence not be separated from the remainder of the AJ143_{—}1 protein.

The EcoRI/NotI restriction fragment obtainable from the deposit containing clone AJ143_{—}1 should be approximately 1000 bp.

The nucleotide sequence disclosed herein for AJ143_{—}1 was searched against the GenBank and GeneSeq nucleotide sequence databases using BLASTN/BLASTX and FASTA search protocols. AJ143_{—}1 demonstrated at least some similarity with sequences identified as T19431 (d08002s Homo sapiens cDNA clone d08002 5′ end) and Z41997 (H. sapiens partial cDNA sequence; clone c-05c07); it may also show some similarity to phosphoenolpyruvate phosphomutase. Based upon sequence similarity, AJ143_{—}1 proteins and each similar protein or peptide may share at least some activity.

Clone “AJ168_{—}4”

A polynucleotide of the present invention has been identified as clone “AJ168_{—}4”. AJ168_{—}4 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AJ168_{—}4 is a full-length clone, including the entire coding sequence of a secreted protein (also referred to herein as “AJ168_{—}4 protein”).

The nucleotide sequence of AJ168_{—}4 as presently determined is reported in SEQ ID NO:112, and includes a poly(A) tail. What applicants presently believe to be the proper reading frame and the predicted amino acid sequence of the AJ168_{—}4 protein corresponding to the foregoing nucleotide sequence is reported in SEQ ID NO:113.

The EcoRI/NotI restriction fragment obtainable from the deposit containing clone AJ168_{—}4 should be approximately 700 bp.

The nucleotide sequence disclosed herein for AJ168_{—}4 was searched against the GenBank and GeneSeq nucleotide sequence databases using BLASTN/BLASTX and FASTA search protocols. AJ168_{—}4 demonstrated at least some similarity with sequences identified as T65223 (yc79c02.s1 Homo sapiens cDNA clone 22106 3′). Based upon sequence similarity, AJ168_{—}4 proteins and each similar protein or peptide may share at least some activity. The TopPredII computer program predicts a potential transmembrane domain within the AJ168_{—}4 protein sequence (SEQ ID NO:113).

Clone “AK684_{—}1”

A polynucleotide of the present invention has been identified as clone “AK684_{—}1”. AK684_{—}1 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AK684_{—}1 is a full-length clone, including the entire coding sequence of a secreted protein (also referred to herein as “AK684_{—}1 protein”).

The nucleotide sequence of AK684_{—}1 as presently determined is reported in SEQ ID NO:114, and includes a poly(A) tail. What applicants presently believe to be the proper reading frame and the predicted amino acid sequence of the AK684_{—}1 protein corresponding to the foregoing nucleotide sequence is reported in SEQ ID NO:115.

The EcoRI/NotI restriction fragment obtainable from the deposit containing clone AK684_{—}1 should be approximately 1000 bp.

The nucleotide sequence disclosed herein for AK684_{—}1 was searched against the GenBank and GeneSeq nucleotide sequence databases using BLASTN/BLASTX and FASTA search protocols. AK684_{—}1 demonstrated at least some similarity with sequences identified as AA226405 (nc20c05.r1 NCI CGAP Pr1 Homo sapiens cDNA clone 2817), G15531 (human STS SHGC-17023), and T68858 (yc30d08.s1 Homo sapiens cDNA clone 82191 3′ similar to contains MSR1 repetitive element). Based upon sequence similarity, AK684_{—}1 proteins and each similar protein or peptide may share at least some activity.

The TopPredII computer program predicts a potential transmembrane domain within the AK684_{—}1 protein sequence centered around amino acid 20 of SEQ ID NO:115.

Clone “AS209_{—}1”

A polynucleotide of the present invention has been identified as clone “AS209_{—}1”. AS209_{—}1 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AS209_{—}1 is a full-length clone, including the entire coding sequence of a secreted protein (also referred to herein as “AS209_{—}1 protein”).

The nucleotide sequence of AS209_{—}1 as presently determined is reported in SEQ ID NO:116, and includes a poly(A) tail. What applicants presently believe to be the proper reading frame and the predicted amino acid sequence of the AS209_{—}1 protein corresponding to the foregoing nucleotide sequence is reported in SEQ ID NO:117. Amino acids 32 to 44 of SEQ ID NO:117 are a predicted leader/signal sequence, with the predicted mature amino acid sequence beginning at amino acid 45. Due to the hydrophobic nature of the predicted leader/signal sequence, it is likely to act as a transmembrane domain should the predicted leader/signal sequence not be separated from the remainder of the AS209_{—}1 protein.

The EcoRI/NotI restriction fragment obtainable from the deposit containing clone AS209_{—}1 should be approximately 2882 bp.

The nucleotide sequence disclosed herein for AS209_{—}1 was searched against the GenBank and GeneSeq nucleotide sequence databases using BLASTN/BLASTX and FASTA search protocols. AS209_{—}1 demonstrated at least some similarity with sequences identified as AA055217 (zf17h02.s1 Soares fetal heart NbHH19W Homo sapiens cDNA clone 377235 3′) and H29533 (ym60h11.r1 Homo sapiens cDNA clone 52955 5′ similar to SP:A60164 S34329; PLATELET MEMBRANE GLYCOPROTEIN V PRECURSOR). The predicted amino acid sequence disclosed herein for AS209_{—}1 was searched against the GenPept and GeneSeq amino acid sequence databases using the BLASTX search protocol. The predicted AS209_{—}1 protein demonstrated at least some similarity with sequences identified as D63875 (ORF [Homo sapiens]) and X53959 (slit protein [Drosophila melanogaster]). Based upon sequence similarity, AS209_{—}1 proteins and each similar protein or peptide may share at least some activity. The TopPredII computer program predicts four potential transmembrane domains within the AS209_{—}1 protein sequence, centered around amino acids 32, 387, 449, and 538 of SEQ ID NO:117, respectively.

AS209_{—}1 protein was expressed in a COS cell expression system, and an expressed protein band of approximately 76 kDa was detected in membrane fractions using SDS polyacrylamide gel electrophoresis.

Clone “AX56_{—}28”

A polynucleotide of the present invention has been identified as clone “AX56_{—}28”. AX56_{—}28 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AX56_{—}28 is a full-length clone, including the entire coding sequence of a secreted protein (also referred to herein as “AX56_{—}28 protein”). The nucleotide sequence of AX56_{—}28 as presently determined is reported in SEQ ID NO:118, and includes a poly(A) tail. What applicants presently believe to be the proper reading frame and the predicted amino acid sequence of the AX56_{—}28 protein corresponding to the foregoing nucleotide sequence is reported in SEQ ID NO:119.

The EcoRI/NotI restriction fragment obtainable from the deposit containing clone AX56_{—}28 should be approximately 4500 bp.

The nucleotide sequence disclosed herein for AX56_{—}28 was searched against the GenBank and GeneSeq nucleotide sequence databases using BLASTN/BLASTX and FASTA search protocols. AX56_{—}28 demonstrated at least some similarity with sequences identified as M20816 (Chicken cytotactin mRNA, partial cds, clone pEC803 [Gallus gallus]), N67571 (yz42a06.s1 Homo sapiens cDNA clone 285682 3′), and T19080 (e05023t Testis 1 Homo sapiens cDNA clone e05023 5′ end). The predicted amino acid sequence disclosed herein for AX56_{—}28 was searched against the GenPept and GeneSeq amino acid sequence databases using the BLASTX search protocol. The predicted AX56_{—}28 protein demonstrated at least some similarity with sequences identified as L12018 (putative protein [Caenorhabditis elegans]). Based upon sequence similarity, AX56_{—}28 proteins and each similar protein or peptide may share at least some activity. The TopPredII computer program predicts a potential transmembrane domain within the AX56_{—}28 protein sequence (SEQ ID NO:119).

AX56_{—}28 protein was expressed in a COS cell expression system, and an expressed protein band of approximately 18 kDa was detected in membrane fractions using SDS polyacrylamide gel electrophoresis.

Clone “AX92_{—}3”

A polynucleotide of the present invention has been identified as clone “AX92_{—}3”. AX92_{—}3 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AX92_{—}3 is a full-length clone, including the entire coding sequence of a secreted protein (also referred to herein as “AX92_{—}3 protein”).

The nucleotide sequence of AX92_{—}3 as presently determined is reported in SEQ ID NO:120, and includes a poly(A) tail. What applicants presently believe to be the proper reading frame and the predicted amino acid sequence of the AX92_{—}3 protein corresponding to the foregoing nucleotide sequence is reported in SEQ ID NO:121. Amino acids 13 to 25 of SEQ ID NO:121 are a predicted leader/signal sequence, with the predicted mature amino acid sequence beginning at amino acid 26. Due to the hydrophobic nature of the predicted leader/signal sequence, it is likely to act as a transmembrane domain should the predicted leader/signal sequence not be separated from the remainder of the AX92_{—}3 protein.

The EcoRI/NotI restriction fragment obtainable from the deposit containing clone AX92_{—}3 should be approximately 1800 bp.

The nucleotide sequence disclosed herein for AX92_{—}3 was searched against the GenBank and GeneSeq nucleotide sequence databases using BLASTN/BLASTX and FASTA search protocols. AX92_{—}3 demonstrated at least some similarity with sequences identified as AA003356 (mg49g01.r1 Soares mouse embryo NbME13.5 14.5 Mus musculus cDNA clone 427152 5′), AA036247 (mi74a03.r1 Soares mouse p3NMF19.5 Mus musculus cDNA clone 472300 5′), F19608 (H. sapiens mitochondrial EST sequence (009-X4-35) from skeletal muscle), M10546 (Human mitochondrial DNA, fragment M1, encoding transfer RNAs, cytochrome oxidase I, and 2 URFs [Mitochondrion Homo sapiens]), and U46493 (Cloning vector pFlp recombinase gene, complete cds). Based upon sequence similarity, AX92_{—}3 proteins and each similar protein or peptide may share at least some activity. The TopPredII computer program predicts four potential transmembrane domains within the AX92_{—}3 protein sequence, centered around amino acids 20, 183, 269, and 295 of SEQ ID NO:121, respectively.

Clone “BF245_{—}1”

A polynucleotide of the present invention has been identified as clone “BF245_{—}1”. BF245_{—}1 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BF245_{—}1 is a full-length clone, including the entire coding sequence of a secreted protein (also referred to herein as “BF245_{—}1 protein”).

The nucleotide sequence of BF245_{—}1 as presently determined is reported in SEQ ID NO:122, and includes a poly(A) tail. What applicants presently believe to be the proper reading frame and the predicted amino acid sequence of the BF245_{—}1 protein corresponding to the foregoing nucleotide sequence is reported in SEQ ID NO:123. Nucleotides 896 to 1294 of SEQ ID NO:122 are presented in the opposite orientation as SEQ ID NO:320; SEQ ID NO:123 is encoded by SEQ ID NO:320. Amino acids 107 to 119 of SEQ ID NO:123 are a predicted leader/signal sequence, with the predicted mature amino acid sequence beginning at amino acid 120. Amino acids 116 to 128 of SEQ ID NO:123 are also a predicted leader/signal sequence, with the predicted mature amino acid sequence beginning in that case at amino acid 129. Due to the hydrophobic nature of these predicted leader/signal sequences, each is likely to act as a transmembrane domain should it not be separated from the remainder of the BF245_{—}1 protein.

The EcoRI/NotI restriction fragment obtainable from the deposit containing clone BF245_{—}1 should be approximately 2300 bp.

The nucleotide sequence disclosed herein for BF245_{—}1 was searched against the GenBank and GeneSeq nucleotide sequence databases using BLASTN/BLASTX and FASTA search protocols. BF245_{—}1 demonstrated at least some similarity with sequences identified as AA001743 (zh86h02.s1 Soares fetal liver spleen 1NFLS S1 Homo sapiens cDNA clone 428211 3′ similar to SW YY02_HUMAN P42285 HYPOTHETICAL MYELOID CELL LINE PROTEIN 2), D29641 (Human mRNA for KIAA0052 gene, partial cds), Q92779 (Human thymopoietin continuous gene fragment), and R39256 (yc91h04.s1 Homo sapiens cDNA clone 23509 3′). The predicted amino acid sequence disclosed herein for BF245_{—}1 was searched against the GenPept and GeneSeq amino acid sequence databases using the BLASTX search protocol. The predicted BF245_{—}1 protein demonstrated at least some similarity with sequences identified as Z70271 (W08D2.7 [Caenorhabditis elegans]). Based upon sequence similarity, BF245_{—}1 proteins and each similar protein or peptide may share at least some activity.

Clone “BG33_{—}7”

A polynucleotide of the present invention has been identified as clone “BG33_{—}7”. BG33_{—}7 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BG33_{—}7 is a full-length clone, including the entire coding sequence of a secreted protein (also referred to herein as “BG33_{—}7 protein”).

The nucleotide sequence of BG33_{—}7 as presently determined is reported in SEQ ID NO:124, and includes a poly(A) tail. What applicants presently believe to be the proper reading frame and the predicted amino acid sequence of the BG33_{—}7 protein corresponding to the foregoing nucleotide sequence is reported in SEQ ID NO:125.

The EcoRI/NotI restriction fragment obtainable from the deposit containing clone BG33_{—}7 should be approximately 900 bp.

The nucleotide sequence disclosed herein for BG33_{—}7 was searched against the GenBank and GeneSeq nucleotide sequence databases using BLASTN/BLASTX and FASTA search protocols. BG33_{—}7 demonstrated at least some similarity with sequences identified as AA033818 (zf02c08.s1 Soares fetal heart NbHH19W Homo sapiens cDNA clone 375758 3′) and AA462657 (vg68e04.r1 Soares mouse NbMH Mus musculus cDNA clone 871134 5′). Based upon sequence similarity, BG33_{—}7 proteins and each similar protein or peptide may share at least some activity.

Clone “BM46_{—}10”

A polynucleotide of the present invention has been identified as clone “BM46_{—}10”. BM46_{—}10 was isolated from a human adult muscle cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BM46_{—}10 is a full-length clone, including the entire coding sequence of a secreted protein (also referred to herein as “BM46_{—}10 protein”).

The nucleotide sequence of BM46_{—}10 as presently determined is reported in SEQ ID NO:126, and includes a poly(A) tail. What applicants presently believe to be the proper reading frame and the predicted amino acid sequence of the BM46_{—}10 protein corresponding to the foregoing, nucleotide sequence is reported in SEQ ID NO:127.

The EcoRI/NotI restriction fragment obtainable from the deposit containing clone BM46_{—}10 should be approximately 3600 bp.

The nucleotide sequence disclosed herein for BM46_{—}10 was searched against the GenBank and GeneSeq nucleotide sequence databases using BLASTN/BLASTX and FASTA search protocols. BM46_{—}10 demonstrated at least some similarity with sequences identified as F19321 (H. sapiens EST sequence 008-X (391 nt)), N79027 (zb43c09.s1 Homo sapiens cDNA clone 306352 3′), U46493 (Cloning vector pFlp recombinase gene, complete cds), and W74198 (zd74d05.s1 Soares fetal heart NbHH19W Homo sapiens cDNA clone 346377 3′). Based upon sequence similarity, BM46_{—}10 proteins and each similar protein or peptide may share at least some activity.

Clone “J317_{—}1”

A polynucleotide of the present invention has been identified as clone “J317_{—}1”. J317_{—}1 was isolated from a human adult blood (peripheral blood mononuclear cells activated by treatment with phytohemagglutinin, phorbol myristate acetate, and mixed lymphocyte reaction) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. J317_{—}1 is a full-length clone, including the entire coding sequence of a secreted protein (also referred to herein as “J317_{—}1 protein”).

The nucleotide sequence of J317_{—}1 as presently determined is reported in SEQ ID NO:128, and includes a poly(A) tail. What applicants presently believe to be the proper reading frame and the predicted amino acid sequence of the J317_{—}1 protein corresponding to the foregoing nucleotide sequence is reported in SEQ ID NO:129. Amino acids 19 to 31 of SEQ ID NO:129 are a predicted leader/signal sequence, with the predicted mature amino acid sequence beginning at amino acid 32. Due to the hydrophobic nature of the predicted leader/signal sequence, it is likely to act as a transmembrane domain should the predicted leader/signal sequence not be separated from the remainder of the J317_{—}1 protein.

The EcoRI/NotI restriction fragment obtainable from the deposit containing clone J317_{—}1 should be approximately 1300 bp.

The nucleotide sequence disclosed herein for J317_{—}1 was searched against the GenBank and GeneSeq nucleotide sequence databases using BLASTN/BLASTX and FASTA search protocols. J317_{—}1 demonstrated at least some similarity with sequences identified as N21491 (yx58f09.s1 Homo sapiens cDNA clone 265961 3′), R39024 (yd08h03.s1 Homo sapiens cDNA clone 25214 3′), T93953 (ye06h06.r1 Homo sapiens cDNA clone 116987 5′ similar to contains HGR repetitive element), and Z25379 (H. sapiens partial cDNA sequence; clone C6F07; version 1; strand(+), single read). Based upon sequence similarity, J317_{—}1 proteins and each similar protein or peptide may share at least some activity.

Clone “O289_{—}1”

A polynucleotide of the present invention has been identified as clone “O289_{—}1”. O289_{—}1 was isolated from a human adult blood (dendritic cells) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. O289_{—}1 is a full-length clone, including the entire coding sequence of a secreted protein (also referred to herein as “O289_{—}1 protein”).

The nucleotide sequence of O289_{—}1 as presently determined is reported in SEQ ID NO:130, and includes a poly(A) tail. What applicants presently believe to be the proper reading frame and the predicted amino acid sequence of the O289_{—}1 protein corresponding to the foregoing nucleotide sequence is reported in SEQ ID NO:131.

The EcoRI/NotI restriction fragment obtainable from the deposit containing clone O289_{—}1 should be approximately 700 bp.

The nucleotide sequence disclosed herein for O289_{—}1 was searched against the GenBank and GeneSeq nucleotide sequence databases using BLASTN/BLASTX and FASTA search protocols. O289_{—}1 demonstrated at least some similarity with sequences identified as H59298 (yr04c07.r1 Homo sapiens cDNA clone 204300 5′ similar to contains MER22 repetitive element). Based upon sequence similarity, O289_{—}1 proteins and each similar protein or peptide may share at least some activity. The TopPredII computer program predicts a large potential transmembrane domain within the O289_{—}1 protein sequence, centered around amino acid 35 of SEQ ID NO:28. The nucleotide sequence of O289_{—}1 indicates that it may contain MER transposon repetitive elements.

Clone “AJ26_{—}3”

A polynucleotide of the present invention has been identified as clone “AJ26_{—}3”. AJ26_{—}3 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AJ26_{—}3 is a full-length clone, including the entire coding sequence of a secreted protein (also referred to herein as “AJ26_{—}3 protein”).

The nucleotide sequence of AJ26_{—}3 as presently determined is reported in SEQ ID NO:132, and includes a poly(A) tail. What applicants presently believe to be the proper reading frame and the predicted amino acid sequence of the AJ26_{—}3 protein corresponding to the foregoing nucleotide sequence is reported in SEQ ID NO:133.

The EcoRI/NotI restriction fragment obtainable from the deposit containing clone AJ26_{—}3 should be approximately 2100 bp.

The nucleotide sequence disclosed herein for AJ26_{—}3 was searched against the GenBank and GeneSeq nucleotide sequence databases using BLASTN/BLASTX and FASTA search protocols. AJ26_{—}3 demonstrated at least some similarity with sequences identified as J01917 (DNA polymerase [Human adenovirus type 2]), J01969 (DNA polymerase [Human adenovirus type 5]), L24893 (HUMAAC02_{—}1 myelin protein zero [Homo sapiens]), U43330 (CTX [Xenopus laevis]), and U43394 (CTX [Xenopus laevis]). Based upon sequence similarity, AJ26_{—}3 proteins and each similar protein or peptide may share at least some activity. The TopPredII computer program predicts four potential transmembrane domains within the AJ26_{—}3 protein sequence, centered around amino acids 11, 41, 163, and 246 of SEQ ID NO:133. The AJ26_{—}3 protein also has a possible signal sequence that could be cleaved to produce a mature protein starting at amino acid 17 of SEQ ID NO:133.

Clone “AJ172_{—}2”

A polynucleotide of the present invention has been identified as clone “AJ172_{—}2”. AJ172_{—}2 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AJ172_{—}2 is a full-length clone, including the entire coding sequence of a secreted protein (also referred to herein as “AJ172_{—}2 protein”).

The nucleotide sequence of AJ172_{—}2 as presently determined is reported in SEQ ID NO:134, and includes a poly(A) tail. What applicants presently believe to be the proper reading frame and the predicted amino acid sequence of the AJ172_{—}2 protein corresponding to the foregoing nucleotide sequence is reported in SEQ ID NO:135. Amino acids 8 to 20 of SEQ ID NO:135 are a predicted leader/signal sequence, with the predicted mature amino acid sequence beginning at amino acid 21. Due to the hydrophobic nature of the predicted leader/signal sequence, it is likely to act as a transmembrane domain should the predicted leader/signal sequence not be separated from the remainder of the AJ172_{—}2 protein.

The EcoRI/NotI restriction fragment obtainable from the deposit containing clone AJ172_{—}2 should be approximately 3000 bp.

The nucleotide sequence disclosed herein for AJ172_{—}2 was searched against the GenBank and GeneSeq nucleotide sequence databases using BLASTN/BLASTX and FASTA search protocols. AJ172_{—}2 demonstrated at least some similarity with sequences identified as AA077794 (7H01C09 Chromosome 7 HeLa cDNA Library Homo sapiens cDNA clone 7H01C09), AC000064 (Human BAC clone RG083M05 from 7q21-7q22, complete sequence), D78692 (Human placenta cDNA 5′-end GEN-503M08), H12439 (yj11h10.r1 Homo sapiens cDNA clone 148483 5′), R27389 (yh46a09.s1 Homo sapiens cDNA clone 132760 3′), and T09280 (Novel AMP/MCF virus clone 24 genome). The predicted amino acid sequence disclosed herein for AJ172_{—}2 was searched against the GenPept and GeneSeq amino acid sequence databases using the BLASTX search protocol. The predicted AJ172_{—}2 protein demonstrated at least some similarity to sequences identified as M26927 (pol polyprotein [Gibbon leukemia virus]), M93134 (pol protein [Friend murine leukemia virus]), and R75189 (Osteoinductive retrovirus RFB-14 pol gene product). AJ172_{—}2 protein is similar to a number of viral env proteins, including those of baboon endogenous virus and many leukemia viruses, which associate with the membrane portion of the viral envelope. Based upon sequence similarity, AJ172_{—}2 proteins and each similar protein or peptide may share at least some activity. The TopPredII computer program predicts five potential transmembrane domains within the AJ172_{—}2 protein sequence, centered around amino acids 104, 267, 292, 328, and 457 of SEQ ID NO:135, respectively.

AJ172_{—}2 protein was expressed in a COS cell expression system, and an expressed protein band of approximately 78 kDa was detected in conditioned medium using SDS polyacrylamide gel electrophoresis.

Clone “AP224_{—}2”

A polynucleotide of the present invention has been identified as clone “AP224_{—}2”. AP224_{—}2 was isolated from a human adult placenta cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AP224_{—}2 is a full-length clone, including the entire coding sequence of a secreted protein (also referred to herein as “AP224_{—}2 protein”).

The nucleotide sequence of AP224_{—}2 as presently determined is reported in SEQ ID NO:136, and includes a poly(A) tail. What applicants presently believe to be the proper reading frame and the predicted amino acid sequence of the AP224_{—}2 protein corresponding to the foregoing nucleotide sequence is reported in SEQ ID NO:137. Amino acids 5 to 17 of SEQ ID NO:137 are a predicted leader/signal sequence, with the predicted mature amino acid sequence beginning at amino acid 18. Due to the hydrophobic nature of the predicted leader/signal sequence, it is likely to act as a transmembrane domain should the predicted leader/signal sequence not be separated from the remainder of the AP224_{—}2 protein.

The EcoRI/NotI restriction fragment obtainable from the deposit containing clone AP224_{—}2 should be approximately 1200 bp.

The nucleotide sequence disclosed herein for AP224_{—}2 was searched against the GenBank and GeneSeq nucleotide sequence databases using BLASTN/BLASTX and FASTA search protocols. AP224_{—}2 demonstrated at least some similarity with sequences identified as R37675 (yf61f08.s1 Homo sapiens cDNA clone 26687 3′). Based upon sequence similarity, AP224_{—}2 proteins and each similar protein or peptide may share at least some activity.

Clone “BL89_{—}13”

A polynucleotide of the present invention has been identified as clone “BL89_{—}13”. BL89_{—}13 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BL89_{—}13 is a full-length clone, including the entire coding sequence of a secreted protein (also referred to herein as “BL89_{—}13 protein”).

The nucleotide sequence of BL89_{—}13 as presently determined is reported in SEQ ID NO:138, and includes a poly(A) tail. What applicants presently believe to be the proper reading frame and the predicted amino acid sequence of the BL89_{—}13 protein corresponding to the foregoing nucleotide sequence is reported in SEQ ID NO:139.

The EcoRI/NotI restriction fragment obtainable from the deposit containing clone BL89_{—}13 should be approximately 3200 bp.

The nucleotide sequence disclosed herein for BL89_{—}13 was searched against the GenBank and GeneSeq nucleotide sequence databases using BLASTN/BLASTX and FASTA search protocols. No hits were found in the database. The TopPredII computer program predicts a potential transmembrane domain within the BL89_{—}13 protein sequence centered around amino acid 625 of SEQ ID NO:139.

Clone “BL341_{—}4”

A polynucleotide of the present invention has been identified as clone “BL341_{—}4”. BL341_{—}4 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BL341_{—}4 is a full-length clone, including the entire coding sequence of a secreted protein (also referred to herein as “BL341_{—}4 protein”).

The nucleotide sequence of BL341_{—}4 as presently determined is reported in SEQ ID NO:140, and includes a poly(A) tail. What applicants presently believe to be the proper reading frame and the predicted amino acid sequence of the BL341_{—}4 protein corresponding to the foregoing nucleotide sequence is reported in SEQ ID NO:141.

The EcoRI/NotI restriction fragment obtainable from the deposit containing clone BL341_{—}4 should be approximately 2600 bp.

The nucleotide sequence disclosed herein for BL341_{—}4 was searched against the GenBank and GeneSeq nucleotide sequence databases using BLASTN/BLASTX and FASTA search protocols. BL341_{—}4 demonstrated at least some similarity with sequences identified as AA460103 (zx50a12.r1 Soares testis NHT Homo sapiens cDNA clone) and Z63359 (H. sapiens CpG island DNA genomic Mse1 fragment, clone 81e7, reverse read cpg81e7.rt1a). Based upon sequence similarity, BL341_{—}4 proteins and each similar protein or peptide may share at least some activity.

Clone “BV239_{—}3”

A polynucleotide of the present invention has been identified as clone “BV239_{—}3”. BV239_{—}3 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BV239_{—}3 is a full-length clone, including the entire coding sequence of a secreted protein (also referred to herein as “BV239_{—}3 protein”).

The nucleotide sequence of BV239_{—}3 as presently determined is reported in SEQ ID NO:142, and includes a poly(A) tail. What applicants presently believe to be the proper reading frame and the predicted amino acid sequence of the BV239_{—}3 protein corresponding to the foregoing nucleotide sequence is reported in SEQ ID NO:143. Amino acids 29 to 41 of SEQ ID NO:143 are a predicted leader/signal sequence, with the predicted mature amino acid sequence beginning at amino acid 42. Due to the hydrophobic nature of the predicted leader/signal sequence, it is likely to act as a transmembrane domain should the predicted leader/signal sequence not be separated from the remainder of the BV239_{—}3 protein.

The EcoRI/NotI restriction fragment obtainable from the deposit containing clone BV239_{—}3 should be approximately 310 bp.

The nucleotide sequence disclosed herein for BV239_{—}3 was searched against the GenBank and GeneSeq nucleotide sequence databases using BLASTN/BLASTX and FASTA search protocols. BV239_{—}3 demonstrated at least some similarity with sequences identified as U46493 (Cloning vector pFlp recombinase gene, complete cds). Based upon sequence similarity, BV239_{—}3 proteins and each similar protein or peptide may share at least some activity.

Clone “CC25_{—}17”

A polynucleotide of the present invention has been identified as clone “CC25_{—}17”. CC25_{—}17 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CC25_{—}17 is a full-length clone, including the entire coding sequence of a secreted protein (also referred to herein as “CC25_{—}17 protein”).

The nucleotide sequence of CC25_{—}17 as presently determined is reported in SEQ ID NO:144, and includes a poly(A) tail. What applicants presently believe to be the proper reading frame and the predicted amino acid sequence of the CC25_{—}17 protein corresponding to the foregoing nucleotide sequence is reported in SEQ ID NO:145.

The EcoRI/NotI restriction fragment obtainable from the deposit containing clone CC25_{—}17 should be approximately 300 bp.

The nucleotide sequence disclosed herein for CC25_{—}17 was searched against the GenBank and GeneSeq nucleotide sequence databases using BLASTN/BLASTX and FASTA search protocols. CC25_{—}17 demonstrated at least some similarity with sequences identified as U46493 (Cloning vector pFlp recombinase gene, complete cds). Based upon sequence similarity, CC25_{—}17 proteins and each similar protein or peptide may share at least some activity.

Clone “CC397_{—}19”

A polynucleotide of the present invention has been identified as clone “CC397_{—}19”. CC397_{—}19 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CC397_{—}19 is a full-length clone, including the entire coding sequence of a secreted protein (also referred to herein as “CC397_{—}19 protein”).

The nucleotide sequence of CC397_{—}19 as presently determined is reported in SEQ ID NO:146, and includes a poly(A) tail. What applicants presently believe to be the proper reading frame and the predicted amino acid sequence of the CC397_{—}19 protein corresponding to the foregoing nucleotide sequence is reported in SEQ ID NO:147.

The EcoRI/NotI restriction fragment obtainable from the deposit containing clone CC397_{—}19 should be approximately 1700 bp.

The nucleotide sequence disclosed herein for CC397_{—}19 was searched against the GenBank and GeneSeq nucleotide sequence databases using BLASTN/BLASTX and FASTA search protocols. CC397_{—}19 demonstrated at least some similarity with sequences identified as AC002129 (Human DNA from chromsome 19 cosmid R33743, genomic sequence, complete sequence), D82019 (Mouse gene for basigin precursor, basigin signal precursor), G08688 (human STS CHLC.GATA29D08.P14592 clone GATA29D08), M68516 (Human protein C inhibitor gene, complete cds), and Z68756 (Human DNA sequence from cosmid L191F1, Huntington's Disease Region, chromosome 4p16.3 contains Huntington Disease (HD) gene, CpG island ESTs and U7 small nuclear RNA). The predicted amino acid sequence disclosed herein for CC397_{—}19 was searched against the GenPept and GeneSeq amino acid sequence databases using the BLASTX search protocol. The predicted CC397_{—}19 protein demonstrated at least some similarity to sequences identified as X52164 (Q300 protein (AA 1-77) [Mus musculus]). Based upon sequence similarity, CC397_{—}19 proteins and each similar protein or peptide may share at least some activity. The nucleotide sequence of CC397_{—}19 indicates that it may contain an Alu repetitive element.

Clone “D305_{—}2”

A polynucleotide of the present invention has been identified as clone “D305_{—}2”. A cDNA clone was first isolated from a human adult blood (peripheral blood mononuclear cells activated by treatment with concanavalin A and phorbol myristate acetate) cDNA library using methods which are selective for cDNAs encoding secreted proteins (see U.S. Pat. No. 5,536,637). This cDNA clone was then used to isolate D305_{—}2 from a human adult blood (peripheral blood mononuclear cells activated by treatment with phytohemagglutinin, phorbol myristate acetate, and mixed lymphocyte reaction) cDNA library. D305_{—}2 is a full-length clone, including the entire coding sequence of a secreted protein (also referred to herein as “D305_{—}2 protein”).

The nucleotide sequence of the 5′ portion of D305_{—}2 as presently determined is reported in SEQ ID NO:148. An additional internal nucleotide sequence from D305_{—}2 as presently determined is reported in SEQ ID NO:149. What applicants believe is the proper reading frame and the predicted amino acid sequence encoded by such internal sequence is reported in SEQ ID NO:150. Amino acids 3 to 15 of SEQ ID NO:150 are a predicted leader/signal sequence, with the predicted mature amino acid sequence being at amino acid 16. Due to the hydrophobic nature of the predicted leader/signal sequence, it is likely to act as a transmembrane domain should the predicted leader/signal sequence not be separated from the remainder of the D305_{—}2 protein. Additional nucleotide sequence from the 3′ portion of D305_{—}2, including a poly(A) tail, is reported in SEQ ID NO:151.

The EcoRI/NotI restriction fragment obtainable from the deposit containing clone D305_{—}2 should be approximately 2400 bp.

The nucleotide sequence disclosed herein for D305_{—}2 was searched against the GenBank and GeneSeq nucleotide sequence databases using BLASTN/BLASTX and FASTA search protocols. D305_{—}2 demonstrated at least some similarity with sequences identified as AA055703 (zl75d04.r1 Stratagene colon (#937204) Homo sapiens cDNA clone 510439 5′), N49593 (yy58d05.s1 Homo sapiens cDNA clone 277737 3′), R66646 (yi35b08.r1 Homo sapiens cDNA clone 141207 5′ similar to SP P24A_YEAST P32802 P24A PROTEIN), U81006 (Human p76 mRNA, complete cds), and Z48758 (S. cerevisiae chromosome IV cosmid 9727). The predicted amino acid sequence disclosed herein for D305_{—}2 was searched against the GenPept and GeneSeq amino acid sequence databases using the BLASTX search protocol. The predicted D305_{—}2 protein demonstrated at least some similarity to sequences identified as U53880 (P24A protein (unknown function) (Swiss Prot. accession number P32802) [Saccharomyces cerevisiae]), U81006 (p76 [Homo sapiens]), X67316 (SCEMP70_{—}1 p24a 70 kDa precursor [Saccharomyces cerevisiae]), and Z48758 (unknown [Saccharomyces cerevisiae]). Based upon sequence similarity, D305_{—}2 proteins and each similar protein or peptide may share at least some activity.

Clone “G55_{—}1”

A polynucleotide of the present invention has been identified as clone “G55_{—}1”. A cDNA clone was first isolated from a human adult blood (peripheral blood mononuclear cells activated by treatment with concanavalin A and phorbol myristate acetate) cDNA library using methods which are selective for cDNAs encoding secreted proteins (see U.S. Pat. No. 5,536,637). This cDNA clone was then used to isolate G55_{—}1 from a human adult blood (peripheral blood mononuclear cells activated by treatment with phytohemagglutinin, phorbol myristate acetate, and mixed lymphocyte reaction) cDNA library. G55_{—}1 is a full-length clone, including the entire coding sequence of a secreted protein (also referred to herein as “G55_{—}1 protein”).

The nucleotide sequence of G55_{—}1 as presently determined is reported in SEQ ID NO:152, and includes a poly(A) tail. What applicants presently believe to be the proper reading frame and the predicted amino acid sequence of the G55_{—}1 protein corresponding to the foregoing nucleotide sequence is reported in SEQ ID NO:153. Amino acids 23 to 35 of SEQ ID NO:153 are a predicted leader/signal sequence, with the predicted mature amino acid sequence beginning at amino acid 36. Amino acids 98 to 110 of SEQ ID NO:153 are also a predicted leader/signal sequence, with the predicted mature amino acid sequence beginning in that case at amino acid 111. Due to the hydrophobic nature of these predicted leader/signal sequences, each is likely to act as a transmembrane domain should it not be separated from the remainder of the G55_{—}1 protein.

The EcoRI/NotI restriction fragment obtainable from the deposit containing clone G55_{—}1 should be approximately 2000 bp.

The nucleotide sequence disclosed herein for G55_{—}1 was searched against the GenBank and GeneSeq nucleotide sequence databases using BLASTN/BLASTX and FASTA search protocols. G55_{—}1 demonstrated at least some similarity with sequences identified as R83586 (yp16a07.r1 Homo sapiens cDNA clone 187572 5′). Based upon sequence similarity, G55_{—}1 proteins and each similar protein or peptide may share at least some activity.

Clone “K39_{—}7”

A polynucleotide of the present invention has been identified as clone “K39_{—}7”. K39_{—}7 was isolated from a Mus musculus adult bone marrow (stromal cell line FCM4) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. K39_{—}7 is a full-length clone, including the entire coding sequence of a secreted protein (also referred to herein as “K39_{—}7 protein”).

The nucleotide sequence of the 5′ portion of K39_{—}7 as presently determined is reported in SEQ ID NO:154. What applicants presently believe is the proper reading frame for the coding region is indicated in SEQ ID NO:155. The predicted amino acid sequence of the K39_{—}7 protein corresponding to the foregoing nucleotide sequence is reported in SEQ ID NO:155. Amino acids 3 to 15 of SEQ ID NO:155 are a predicted leader/signal sequence, with the predicted mature amino acid sequence beginning at amino acid 16. Due to the hydrophobic nature of the predicted leader/signal sequence, it is likely to act as a transmembrane domain should the predicted leader/signal sequence not be separated from the remainder of the K39_{—}7 protein. Additional nucleotide sequence from the 3′ portion of K39_{—}7, including a poly(A) tail, is reported in SEQ ID NO:156.

The EcoRI/NotI restriction fragment obtainable from the deposit containing clone K39_{—}7 should be approximately 1675 bp.

The nucleotide sequence disclosed herein for K39_{—}7 was searched against the GenBank and GeneSeq nucleotide sequence databases using BLASTN/BLASTX and FASTA search protocols. K39_{—}7 demonstrated at least some similarity with sequences identified as AA254326 (va15d06.r1 Soares mouse lymph node NbMLN Mus musculus cDNA clone 722987 5′ similar to WP:C09G4.1 CE03978), D18935 (Mouse 3′-directed cDNA, MUSGS01125, clone mc0564), H14129 (ym65b04.r1 Homo sapiens cDNA clone 163759 5′), and R20230 (hUOG-1, DNA segment encoding a mammalian GDF-1 protein). The predicted amino acid sequence disclosed herein for K39_{—}7 was searched against the GenPept and GeneSeq amino acid sequence databases using the BLASTX search protocol. The predicted K39_{—}7 protein demonstrated at least some similarity to sequences identified as R86811 (Saccharomyces cerevisiae mutant LAG1 protein) and U42438 (similar to S. cerevisiae longevity-assurance protein 1 (SP P38703) [Caenorhabditis elegans]). K39_{—}7 nucleotide and protein sequences also demonstrate at least some similarity to those of human clone D147_{—}17 described above. Based upon sequence similarity, K39_{—}7 proteins and each similar protein or peptide may share at least some activity.

Clone “K330_{—}3”

A polynucleotide of the present invention has been identified as clone “K330_{—}3”. K330_{—}3 was isolated from a Mus musculus adult bone marrow (stromal cell line FCM-4) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. K330_{—}3 is a full-length clone, including the entire coding sequence of a secreted protein (also referred to herein as “K330_{—}3 protein”).

The nucleotide sequence of K330_{—}3 as presently determined is reported in SEQ ID NO:157, and includes a poly(A) tail. What applicants presently believe to be the proper reading frame and the predicted amino acid sequence of the K330_{—}3 protein corresponding to the foregoing nucleotide sequence is reported in SEQ ID NO:158.

The EcoRI/NotI restriction fragment obtainable from the deposit containing clone K330_{—}3 should be approximately 1300 bp.

The nucleotide sequence disclosed herein for K330_{—}3 was searched against the GenBank and GeneSeq nucleotide sequence databases using BLASTN/BLASTX and FASTA search protocols. K330_{—}3 demonstrated at least some similarity with sequences identified as A03900 (H. sapiens HuV(NP) gene), AA038010 (mi80a11.r1 Soares mouse p3NMF19.5 Mus musculus cDNA clone 472892 5′), M30775 (Mouse thymidylate synthase pseudogene, 3′ flank), R40824 (yf82c07.s1 Homo sapiens cDNA clone 28939 3′), T23245 (Human gene signature HUMGS05046), and U23512 (Caenorhabditis elegans cosmid M01G4). Based upon sequence similarity, K330_{—}3 proteins and each similar protein or peptide may share at least some activity. The N-terminal portion of the K330_{—}3 protein sequence is hydrophobic. The nucleotide sequence of K330_{—}3 indicates that it may contain CAAAA repeats.

Clone “K363_{—}3”

A polynucleotide of the present invention has been identified as clone “K363_{—}3”. K363_{—}3 was isolated from a Mus musculus adult bone marrow (stromal cell line FCM-4) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. K363_{—}3 is a full-length clone, including the entire coding sequence of a secreted protein (also referred to herein as “K363_{—}3 protein”).

The nucleotide sequence of K363_{—}3 as presently determined is reported in SEQ ID NO:159, and includes a poly(A) tail. What applicants presently believe to be the proper reading frame and the predicted amino acid sequence of the K363_{—}3 protein corresponding to the foregoing nucleotide sequence is reported in SEQ ID NO:160. Amino acids 215 to 227 of SEQ ID NO:160 are a predicted leader/signal sequence, with the predicted mature amino acid sequence beginning at amino acid 228. Due to the hydrophobic nature of the predicted leader/signal sequence, it is likely to act as a transmembrane domain should the predicted leader/signal sequence not be separated from the remainder of the K363_{—}3 protein.

The EcoRI/NotI restriction fragment obtainable from the deposit containing clone K363_{—}3 should be approximately 2690 bp.

The nucleotide sequence disclosed herein for K363_{—}3 was searched against the GenBank and GeneSeq nucleotide sequence databases using BLASTN/BLASTX and FASTA search protocols. K363_{—}3 demonstrated at least some similarity with sequences identified as AA437876 (vd20h06.s1 Knowles Solter mouse 2 cell Mus musculus cDNA clone 793115 5′), AF002668 (Homo sapiens putative fatty acid desaturase MLD mRNA, complete cds), D21554 (Mouse embryonal carcinoma F9 cell cDNA, 67F09), and Y08460 (Mus musculus mRNA for Mdes transmembrane protein). The predicted amino acid sequence disclosed herein for K363_{—}3 was searched against the GenPept and GeneSeq amino acid sequence databases using the BLASTX search protocol. The predicted K363_{—}3 protein demonstrated at least some similarity to sequences identified as Y08460 (Mdes protein [Mus musculus]). Based upon sequence similarity, K363_{—}3 proteins and each similar protein or peptide may share at least some activity. The TopPredII computer program predicts three potential transmembrane domains within the K363_{—}3 protein sequence, centered around amino acids 51, 78, and 228 of SEQ ID NO:160, respectively.

Clone “K446_{—}3”

A polynucleotide of the present invention has been identified as clone “K446_{—}3”. K446_{—}3 was isolated from a Mus musculus adult bone marrow (stromal cell line FCM4) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. K446_{—}3 is a full-length clone, including the entire coding sequence of a secreted protein (also referred to herein as “K446_{—}3 protein”).

The nucleotide sequence of K446_{—}3 as presently determined is reported in SEQ ID NO:161, and includes a poly(A) tail. What applicants presently believe to be the proper reading frame and the predicted amino acid sequence of the K446_{—}3 protein corresponding to the foregoing nucleotide sequence is reported in SEQ ID NO:162.

The EcoRI/NotI restriction fragment obtainable from the deposit containing clone K446_{—}3 should be approximately 2150 bp.

The nucleotide sequence disclosed herein for K446_{—}3 was searched against the GenBank and GeneSeq nucleotide sequence databases using BLASTN/BLASTX and FASTA search protocols. No hits were found in the database.

Clone “K464_{—}4”

A polynucleotide of the present invention has been identified as clone “K464_{—}4”. K464_{—}4 was isolated from a Mus musculus adult bone marrow (stromal cell line FCM4) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. K464_{—}4 is a full-length clone, including the entire coding sequence of a secreted protein (also referred to herein as “K464_{—}4 protein”).

The nucleotide sequence of K464_{—}4 as presently determined is reported in SEQ ID NO:163, and includes a poly(A) tail. What applicants presently believe to be the proper reading frame and the predicted amino acid sequence of the K464_{—}4 protein corresponding to the foregoing nucleotide sequence is reported in SEQ ID NO:164.

The EcoRI/NotI restriction fragment obtainable from the deposit containing clone K464_{—}4 should be approximately 1250 bp.

The nucleotide sequence disclosed herein for K464_{—}4 was searched against the GenBank and GeneSeq nucleotide sequence databases using BLASTN/BLASTX and FASTA search protocols. K464_{—}4 demonstrated at least some similarity with sequences identified as AA260484 (va95a09.r1 Soares mouse NML Mus musculus cDNA clone 747160 5′), AA419864 (vf49b08.r1 Soares mouse NbM), L25338 (Mus musculus folate-binding protein gene, 5′ end), M22527 (Mouse cytotoxic T lymphocyte-specific serine protease), T01176 (P815A antigen precursor gene P1A), T21224 (Human gene signature HUMGS02538), T41900 (Vector pAPEX-3p), U46493 (Cloning vector pFlp recombinase gene, complete cds), U89673 (Cloning vector pIRES1neo, complete plasmid sequence), W32699 (zc06b11.s1 Soares parathyroid tumor NbHPA Homo sapiens cDNA clone 321501 3′), and W36926 (mb82b10.r1 Soares mouse p3NMF19.5 Mus musculus cDNA clone 335899 5′). The predicted amino acid sequence disclosed herein for K464_{—}4 was searched against the GenPept and GeneSeq amino acid sequence databases using the BLASTX search protocol. The predicted K464_{—}4 protein demonstrated at least some similarity to sequences identified as L33768 (JAK3 [Mus musculus]) and X16213 (MHC T7 class I antigen (64 AA) (119 is 2nd base in codon) [Mus musculus]). Based upon sequence similarity, K464_{—}4 proteins and each similar protein or peptide may share at least some activity.

Clone “K483_{—}1”

A polynucleotide of the present invention has been identified as clone “K483_{—}1”. K483_{—}1 was isolated from a Mus musculus adult bone marrow (stromal cell line FCM4) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. K483_{—}1 is a full-length clone, including the entire coding sequence of a secreted protein (also referred to herein as “K483_{—}1 protein”). The human cDNA clone corresponding to K483_{—}1, CH4_{—}11, is described in WO 99/38959.

The nucleotide sequence of K483_{—}1 as presently determined is reported in SEQ ID NO:165, and includes a poly(A) tail. What applicants presently believe to be the proper reading frame and the predicted amino acid sequence of the K483_{—}1 protein corresponding to the foregoing nucleotide sequence is reported in SEQ ID NO:166. Amino acids 184 to 196 of SEQ ID NO:166 are a predicted leader/signal sequence, with the predicted mature amino acid sequence beginning at amino acid 197. Due to the hydrophobic nature of the predicted leader/signal sequence, it is likely to act as a transmembrane domain should the predicted leader/signal sequence not be separated from the remainder of the K483_{—}1 protein.

The EcoRI/NotI restriction fragment obtainable from the deposit containing clone K483_{—}1 should be approximately 1500 bp.

The nucleotide sequence disclosed herein for K483_{—}1 was searched against the GenBank and GeneSeq nucleotide sequence databases using BLASTN/BLASTX and FASTA search protocols. K483_{—}1 demonstrated at least some similarity with sequences identified as AA110914 (mm02c03.r1 Stratagene mouse kidney (#937315) Mus musculus cDNA clone 520324 5′), AA318160 (EST20431 Retina II Homo sapiens cDNA 5′ end), AA500150 (vi97c09.r1 Barstead mouse pooled organs MPLRB4 Mus musculus cDNA clone 920176 5′), and N41895 (yw86b03.r1 Homo sapiens cDNA clone 259085 5′). Based upon sequence similarity, K483_{—}1 proteins and each similar protein or peptide may share at least some activity. The TopPredII computer program predicts three potential transmembrane domains within the K483_{—}1 protein sequence, centered around amino acids 18, 179, and 270 of SEQ ID NO:166, respectively. The K483_{—}1 protein also has a possible signal sequence that could be cleaved to produce a mature protein starting at amino acid 34 of SEQ ID NO:166.

Clone “L69_{—}3”

A polynucleotide of the present invention has been identified as clone “L69_{—}3”. L69_{—}3 was isolated from a Mus musculus adult thymus cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. L69_{—}3 is a full-length clone, including the entire coding sequence of a secreted protein (also referred to herein as “L69_{—}3 protein”).

The nucleotide sequence of L69_{—}3 as presently determined is reported in SEQ ID NO:167, and includes a poly(A) tail. What applicants presently believe to be the proper reading frame and the predicted amino acid sequence of the L69_{—}3 protein corresponding to the foregoing nucleotide sequence is reported in SEQ ID NO:168. Amino acids 4 to 16 of SEQ ID NO:168 are a predicted leader/signal sequence, with the predicted mature amino acid sequence beginning at amino acid 17. Due to the hydrophobic nature of the predicted leader/signal sequence, it is likely to act as a transmembrane domain should the predicted leader/signal sequence not be separated from the remainder of the L69_{—}3 protein.

The EcoRI/NotI restriction fragment obtainable from the deposit containing clone L69_{—}3 should be approximately 1200 bp.

The nucleotide sequence disclosed herein for L69_{—}3 was searched against the GenBank and GeneSeq nucleotide sequence databases using BLASTN/BLASTX and FASTA search protocols. L69_{—}3 demonstrated at least some similarity with sequences identified as H35162 (EST108034 Rattus sp. cDNA similar to H. sapiens hypothetical protein (PIR:S25641)), U02442 (Cloning vector pADbeta, complete sequence), W74864 (md91b10.r1 Soares mouse embryo NbME13.5 14.5 Mus musculus cDNA), and X67698 (H. sapiens tissue specific mRNA). The predicted amino acid sequence disclosed herein for L69_{—}3 was searched against the GenPept and GeneSeq amino acid sequence databases using the BLASTX search protocol. The predicted L69_{—}3 protein demonstrated at least some similarity to sequences identified as A18921 (tissue-specific secretory protein [unidentified]) and X78134 (epididymal secretory protein 14.6 [Macaca fascicularis]). Based upon sequence similarity, L69_{—}3 proteins and each similar protein or peptide may share at least some activity.

L69_{—}3 protein was expressed in a COS cell expression system, and an expressed protein band of approximately 24 kDa was detected in conditioned medium and membrane fractions using SDS polyacrylamide gel electrophoresis.

Clone “BG511_{—}30”

A polynucleotide of the present invention has been identified as clone “BG511_{—}30”. BG511_{—}30 was isolated from a human adult brain cDNA library using methods which are selective for cDNAs encoding secreted proteins (see U.S. Pat. No. 5,536,637), or was identified as encoding, a secreted or transmembrane protein on the basis of computer analysis of the amino acid sequence of the encoded protein. BG511_{—}30 is a full-length clone, including the entire coding sequence of a secreted protein (also referred to herein as “BG511_{—}30 protein”).

The nucleotide sequence of BG511_{—}30 as presently determined is reported in SEQ ID NO:169, and includes a poly (A) tail. What applicants presently believe to be the proper reading frame and the predicted amino acid sequence of the BG511_{—}30 protein corresponding to the foregoing nucleotide sequence is reported in SEQ ID NO:170.

The EcoRI/NotI restriction fragment obtainable from the deposit containing clone BG511_{—}30 should be approximately 3000 bp.

The nucleotide sequence disclosed herein for BG511_{—}30 was searched against the GenBank and GeneSeq nucleotide sequence databases using BLASTN/BLASTX and FASTA search protocols. BG511_{—}30 demonstrated at least some similarity with sequences identified as AA013272 (ze29d03.s1 Soares retina N2b4HR Homo sapiens cDNA clone 360389 3′), AA019923 (ze63h01.s1 Soares retina N2b4HR Homo sapiens cDNA clone 363697 3′), L29074 (Homo sapiens fragile X mental retardation protein (FMR-1) gene (6 alternative splices), complete cds), Q76950 (Human genome fragment), R52631 (yg82c07.s1 Homo sapiens cDNA clone 40006 3′ similar to contains MER14 repetitive element), T41078 (ya30c02.s5 Homo sapiens cDNA clone 62114 3′), and Z73913 (Human DNA sequence from cosmid U61B11, between markers DXS366 and DXS87 on chromosome X contains ESTs). Based upon sequence similarity, BG511_{—}30 proteins and each similar protein or peptide may share at least some activity. The TopPredII computer program predicts three potential transmembrane domains within the BG511_{—}30 protein sequence, centered around amino acids 23, 49, and 97 of SEQ ID NO:170, respectively.

Clone “BL15_{—}12”

A polynucleotide of the present invention has been identified as clone “BL15_{—}12”. BL15_{—}12 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BL15_{—}12 is a full-length clone, including the entire coding sequence of a secreted protein (also referred to herein as “BL15_{—}12 protein”).

The nucleotide sequence of BL15_{—}12 as presently determined is reported in SEQ ID NO:171, and includes a poly(A) tail. What applicants presently believe to be the proper reading frame and the predicted amino acid sequence of the BL15_{—}12 protein corresponding to the foregoing nucleotide sequence is reported in SEQ ID NO:172.

The EcoRI/NotI restriction fragment obtainable from the deposit containing clone BL15_{—}12 should be approximately 1300 bp.

The nucleotide sequence disclosed herein for BL15_{—}12 was searched against the GenBank and GeneSeq nucleotide sequence databases using BLASTN/BLASTX and FASTA search protocols. No hits were found in the database. The TopPredII computer program predicts a potential transmembrane domain within the BL15_{—}12 protein sequence centered around amino acid 35 of SEQ ID NO:172.

Clone “K289_{—}4”

A polynucleotide of the present invention has been identified as clone “K289_{—}4”. K289_{—}4 was isolated from a Mus musculus adult bone marrow (stromal cell line FCM-4) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. K289_{—}4 is a full-length clone, including the entire coding sequence of a secreted protein (also referred to herein as “K289_{—}4 protein”).

The nucleotide sequence of K289_{—}4 as presently determined is reported in SEQ ID NO:173, and includes a poly(A) tail. What applicants presently believe to be the proper reading frame and the predicted amino acid sequence of the K289_{—}4 protein corresponding to the foregoing nucleotide sequence is reported in SEQ ID NO:174. Amino acids 8 to 20 of SEQ ID NO:174 are a predicted leader/signal sequence, with the predicted mature amino acid sequence beginning at amino acid 21. Due to the hydrophobic nature of the predicted leader/signal sequence, it is likely to act as a transmembrane domain should the predicted leader/signal sequence not be separated from the remainder of the K289_{—}4 protein.

The EcoRI/NotI restriction fragment obtainable from the deposit containing clone K289_{—}4 should be approximately 2000 bp.

The nucleotide sequence disclosed herein for K289_{—}4 was searched against the GenBank and GeneSeq nucleotide sequence databases using BLASTN/BLASTX and FASTA search protocols. K289_{—}4 demonstrated at least some similarity with sequences identified as AA183558 (mo97b06.r1 Stratagene mouse testis (#937308) Mus musculus cDNA clone IMAGE:567635 5′ similar to WP:F42G9.8 CE07235; mRNA sequence), AA184674 (mt58h08.r1 Soares 2NbMT Mus musculus cDNA clone 634143 5′), AF038008 (Mus musculus tyrosylprotein sulfotransferase-1 mRNA, complete cds), R75054 (MDB0966 Mus musculus cDNA 3′end), T93946 (ye06g02.r1 Homo sapiens cDNA clone 116978 5′), U07648 (Cloning vector pPUR, complete sequence), and W77682 (me82d12.r1 Soares mouse embryo NbME13.5 14.5 Mus musculus cDNA clone 402071 5)′. TPST-1 catalyzes the transfer of sulfate from 3′-phosphoadenosine 5′-phosphosulfate to tyrosine residues within acidic motifs of polypeptides. Tyrosine O-sulfation has been shown to be important in protein-protein interactions in several systems. For example, sulfation of tyrosine residues in the leukocyte adhesion molecule P-selectin glycoprotein ligand 1 (PSGL-1) is required for binding to P-selectin on activated endothelium (Ouyang et al., 1998, Proc Natl Acad Sci USA 95(6): 2896-2901; which is incorporated by reference herein). Based upon sequence similarity, K289_{—}4 proteins and each similar protein or peptide may share at least some activity.

K289_{—}4 protein was expressed in a COS cell expression system, and an expressed protein band of approximately 47 kDa was detected in conditioned medium and membrane fractions using SDS polyacrylamide gel electrophoresis.

Clone “K322 4”

A polynucleotide of the present invention has been identified as clone “K322_{—}4”.

K322_{—}4 was isolated from a Mus musculus adult bone marrow (stromal cell line FCM4) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. K322_{—}4 is a full-length clone, including the entire coding sequence of a secreted protein (also referred to herein as “K322_{—}4 protein”).

The nucleotide sequence of K322_{—}4 as presently determined is reported in SEQ ID NO:175, and includes a poly(A) tail. What applicants presently believe to be the proper reading frame and the predicted amino acid sequence of the K322_{—}4 protein corresponding to the foregoing nucleotide sequence is reported in SEQ ID NO:176. Amino acids 109 to 121 of SEQ ID NO:176 are a predicted leader/signal sequence, with the predicted mature amino acid sequence beginning at amino acid 122. Due to the hydrophobic nature of the predicted leader/signal sequence, it is likely to act as a transmembrane domain should the predicted leader/signal sequence not be separated from the remainder of the K322_{—}4 protein.

The EcoRI/NotI restriction fragment obtainable from the deposit containing clone K322_{—}4 should be approximately 1300 bp.

The nucleotide sequence disclosed herein for K322_{—}4 was searched against the GenBank and GeneSeq nucleotide sequence databases using BLASTN/BLASTX and FASTA search protocols. K322_{—}4 demonstrated at least some similarity with sequences identified as AA047913 (mj25d03.r1 Soares mouse embryo NbME13.5 14.5 Mus musculus cDNA clone 477125 5′) and W15869 (mb55a11.r1 Soares mouse p3NM19.5 Mus musculus cDNA clone 333308). The predicted amino acid sequence disclosed herein for K322_{—}4 was searched against the GenPept and GeneSeq amino acid sequence databases using the BLASTX search protocol. The predicted K322_{—}4 protein demonstrated at least some similarity to sequences identified as AF045646 (contains similarity to human cyclin A/CDK2-associated protein p19, an RNA polymerase II elongation factor-like protein (GB:U33760) [Caenorhabditis elegans]) and L00089 (Ig v-t1 precursor [Mus musculus]). Based upon sequence similarity, K322_{—}4 proteins and each similar protein or peptide may share at least some activity. The TopPredII computer program predicts a potential transmembrane domain within the K322_{—}4 protein sequence, centered around amino acid 117 of SEQ ID NO:176.

Clone “AM349_{—}2”

A polynucleotide of the present invention has been identified as clone “AM349_{—}2”. AM349_{—}2 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AM349_{—}2 is a full-length clone, including the entire coding sequence of a secreted protein (also referred to herein as “AM349_{—}2 protein”).

The nucleotide sequence of AM349_{—}2 as presently determined is reported in SEQ ID NO:177, and includes a poly(A) tail. What applicants presently believe to be the proper reading frame and the predicted amino acid sequence of the AM349_{—}2 protein corresponding to the foregoing nucleotide sequence is reported in SEQ ID NO:178. Amino acids 22 to 34 of SEQ ID NO:178 are a predicted leader/signal sequence, with the predicted mature amino acid sequence beginning at amino acid 35. Due to the hydrophobic nature of the predicted leader/signal sequence, it is likely to act as a transmembrane domain should the predicted leader/signal sequence not be separated from the remainder of the AM349_{—}2 protein.

The EcoRI/NotI restriction fragment obtainable from the deposit containing clone AM349_{—}2 should be approximately 3400 bp.

The nucleotide sequence disclosed herein for AM349_{—}2 was searched against the GenBank and GeneSeq nucleotide sequence databases using BLASTN/BLASTX and FASTA search protocols. AM349_{—}2 demonstrated at least some similarity with sequences identified as AA078927 (zm92a08.s1 Stratagene ovarian cancer (#937219) Homo sapiens cDNA clone 545366 3′), H06061 (yl72e10.s1 Homo sapiens cDNA clone 43276 3′), U46493 (Cloning vector pFlp recombinase gene, complete cds), W81648 (zd84d09.r1 Soares fetal heart NbHH19W Homo sapiens cDNA clone 347345 5′), and W81649 (zd84d09.s1 Soares fetal heart NbHH19W Homo sapiens cDNA clone 347345 3′). The predicted amino acid sequence disclosed herein for AM349_{—}2 was searched against the GenPept and GeneSeq amino acid sequence databases using the BLASTX search protocol. The predicted AM349_{—}2 protein demonstrated at least some similarity to sequences identified as J01969 (DNA polymerase [Human adenovirus type 5]) and X59599 (protein-tyrosine phosphatase). Based upon sequence similarity, AM349_{—}2 proteins and each similar protein or peptide may share at least some activity.

AM349_{—}2 protein was expressed in a COS cell expression system, and an expressed protein band of approximately 60 kDa was detected in membrane fractions using SDS polyacrylamide gel electrophoresis.

Clone “AR310_{—}3”

A polynucleotide of the present invention has been identified as clone “AR310_{—}3”. AR310_{—}3 wa3 isolated from a human adult retina cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AR310_{—}3 is a full-length clone, including the entire coding sequence of a secreted protein (also referred to herein as “AR310_{—}3 protein”).

The nucleotide sequence of AR310_{—}3 as presently determined is reported in SEQ ID NO:179, and includes a poly(A) tail. What applicants presently believe to be the proper reading frame and the predicted amino acid sequence of the AR310_{—}3 protein corresponding to the foregoing nucleotide sequence is reported in SEQ ID NO:180.

The EcoRI/NotI restriction fragment obtainable from the deposit containing clone AR310_{—}3 should be approximately 3800 bp.

The nucleotide sequence disclosed herein for AR310_{—}3 was searched against the GenBank and GeneSeq nucleotide sequence databases using BLASTN/BLASTX and FASTA search protocols. AR310_{—}3 demonstrated at least some similarity with sequences identified as AA313755 (EST185840 Colon carcinoma (HCC) cell line II Homo sapiens cDNA 5′ end), N35123 (yy20b01.s1 Homo sapiens cDNA clone 271753 3′), N36408 (yy33f03.s1 Homo sapiens cDNA clone 273053 3′), W61057 (zc54a11.r1 Soares senescent fibroblasts NbHSF Homo sapiens cDNA clone 326108 5′ similar to contains element MSR1 repetitive element), and X16706 (Human fra-2 mRNA). Based upon sequence similarity, AR310_{—}3 proteins and each similar protein or peptide may share at least some activity. The TopPredII computer program predicts a potential transmembrane domain within the AR310_{—}3 protein sequence centered around amino acid 66 of SEQ ID NO:180; this region is also a possible signal sequence.

Clone “AS186_{—}3”

A polynucleotide of the present invention has been identified as clone “AS186_{—}3”. AS186_{—}3 was isolated from a human fetal brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AS186_{—}3 is a full-length clone, including the entire coding sequence of a secreted protein (also referred to herein as “AS186_{—}3 protein”).

The nucleotide sequence of AS186_{—}3 as presently determined is reported in SEQ ID NO:181, and includes a poly(A) tail. What applicants presently believe to be the proper reading frame and the predicted amino acid sequence of the AS186_{—}3 protein corresponding to the foregoing nucleotide sequence is reported in SEQ ID NO:182. Amino acids 21 to 33 of SEQ ID NO:182 are a predicted leader/signal sequence, with the predicted mature amino acid sequence beginning at amino acid 34. Due to the hydrophobic nature of the predicted leader/signal sequence, it is likely to act as a transmembrane domain should the predicted leader/signal sequence not be separated from the remainder of the AS186_{—}3 protein.

The EcoRI/NotI restriction fragment obtainable from the deposit containing clone AS186_{—}3 should be approximately 1200 bp.

The nucleotide sequence disclosed herein for AS186_{—}3 was searched against the GenBank and GeneSeq nucleotide sequence databases using BLASTN/BLASTX and FASTA search protocols. AS186_{—}3 demonstrated at least some similarity with sequences identified as J00083 (Human Alu family interspersed repeat; clone BLUR11) and U14574 (***ALU WARNING Human Alu-Sx subfamily consensus sequence). The predicted amino acid sequence disclosed herein for AS186_{—}3 was searched against the GenPept and GeneSeq amino acid sequence databases using the BLASTX search protocol. The predicted AS186_{—}3 protein demonstrated at least some similarity to sequences identified as S58722 (X-linked retinopathy protein {C-terminal, clone XEH.8c} [human]). Based upon sequence similarity, AS186_{—}3 proteins and each similar protein or peptide may share at least some activity. The nucleotide sequence of AS186_{—}3 indicates that it may contain an Alu repetitive element.

Clone “AY160_{—}2”

A polynucleotide of the present invention has been identified as clone “AY160_{—}2”. AY160_{—}2 was isolated from a human adult retina cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AY160_{—}2 is a full-length clone, including the entire coding sequence of a secreted protein (also referred to herein as “AY160_{—}2 protein”).

The nucleotide sequence of AY160_{—}2 as presently determined is reported in SEQ ID NO:183, and includes a poly(A) tail. What applicants presently believe to be the proper reading frame and the predicted amino acid sequence of the AY160_{—}2 protein corresponding to the foregoing nucleotide sequence is reported in SEQ ID NO:184. Amino acids 3 to 15 of SEQ ID NO:184 are a predicted leader/signal sequence, with the predicted mature amino acid sequence beginning at amino acid 16. Due to the hydrophobic nature of the predicted leader/signal sequence, it is likely to act as a transmembrane domain should the predicted leader/signal sequence not be separated from the remainder of the AY160_{—}2 protein.

The EcoRI/NotI restriction fragment obtainable from the deposit containing clone AY160_{—}2 should be approximately 1900 bp.

The nucleotide sequence disclosed herein for AY160_{—}2 was searched against the GenBank and GeneSeq nucleotide sequence databases using BLASTN/BLASTX and FASTA search protocols. AY160_{—}2 demonstrated at least some similarity with sequences identified as D12485 (Human mRNA for nucleotide pyrophosphatase, complete cds), D30649 (Rat mRNA for phosphodiesterase I, complete cds), N77069 (yz84h12.r1 Homo sapiens cDNA clone 289799 5′), and Z47987 (R. norvegicus mRNA for RB13-6 antigen). The predicted amino acid sequence disclosed herein for AY160_{—}2 was searched against the GenPept and GeneSeq amino acid sequence databases using the BLASTX search protocol. The predicted AY160_{—}2 protein demonstrated at least some similarity to sequences identified as D12485 (HUMNPP_{—}2 NPPase [Homo sapiens]), D30649 (phosphodiesterase I [Rattus rattus]), and Z47987 (RNRB13×6_{—}1 RB13-6 antigen [Rattus norvegicus]). The AY160_{—}2 protein also has some domains of sequence similarity to a variety of integral membrane proteins including glycoprotein PC-1. Based upon sequence similarity, AY160_{—}2 proteins and each similar protein or peptide may share at least some activity. The TopPredII computer program predicts a potential transmembrane domain near the carboxy terminus of the AY160_{—}2 protein, around amino acid 418 of SEQ ID NO:184.

AY160_{—}2 protein was expressed in a COS cell expression system, and an expressed protein band of approximately 45 kDa was detected in membrane fractions using SDS polyacrylamide gel electrophoresis.

Clone “BD127_{—}16”

A polynucleotide of the present invention has been identified as clone “BD127_{—}16”. BD127_{—}16 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BD127_{—}16 is a full-length clone, including the entire coding sequence of a secreted protein (also referred to herein as “BD127_{—}16 protein”).

The nucleotide sequence of BD127_{—}16 as presently determined is reported in SEQ ID NO:185, and includes a poly(A) tail. What applicants presently believe to be the proper reading frame and the predicted amino acid sequence of the BD127_{—}16 protein corresponding to the foregoing nucleotide sequence is reported in SEQ ID NO:186.

The EcoRI/NotI restriction fragment obtainable from the deposit containing clone BD127_{—}16 should be approximately 1080 bp.

The nucleotide sequence disclosed herein for BD127_{—}16 was searched against the GenBank and GeneSeq nucleotide sequence databases using BLASTN/BLASTX and FASTA search protocols. BD127_{—}16 demonstrated at least some similarity with sequences identified as AA589375 (vl46c07.s1 Stratagene mouse skin (#937313) Mus musculus cDNA clone IMAGE:975276 3′ similar to TR:G726324 G726324 FIBRILLIN-1; mRNA sequence), M55683 (Human cartilage matrix protein), and U69262 (Mus musculus matrilin-2 precursor mRNA, complete cds). The predicted amino acid sequence disclosed herein for BD127_{—}16 was searched against the GenPept and GeneSeq amino acid sequence databases using the BLASTX search protocol. The predicted BD127_{—}16 protein demonstrated at least some similarity to sequences identified as U03272 (HSU03272_{—}1 fibrillin-2 [Homo sapiens]), U69262 (matrilin-2 precursor [Mus musculus]), and X04571 (HSEGFRER_{—}1 Human mRNA for kidney epidermal growth factor (EGF) precursor [Homo sapiens]). Based upon sequence similarity, BD127_{—}16 proteins and each similar protein or peptide may share at least some activity. Motifs analysis has revealed the presence of two aspartic acid and asparagine hydroxylation sites in both BD127_{—}16 protein and the murine matrilin-2 precursor in the region of similarity between them. Post-translational hydroxylation of aspartic acid or asparagine to form erythro-beta-hydroxyaspartic acid or erythro-beta-hydroxyasparagine has been identified in a number of proteins with domains homologous to epidermal growth factor (EGF). Hidden markov model analysis has revealed the presence of two EGF-like domains in BD127_{—}16 protein at residues 46 to 81 and 87 to 122 of SEQ ID NO:186 (this is a region of particular similarity between BD127_{—}16 protein and the murine matrilin-2 precursor). The TopPredII computer program predicts two potential transmembrane domains within the BD127_{—}16 protein sequence, one centered around amino acid 20 and another around amino acid 150 of SEQ ID NO:186. Therefore, BD127_{—}16 protein appears to be a novel membrane protein containing EGF-like repeats. The nucleotide sequence of BD127_{—}16 indicates that it may contain a PTR7 repetitive element.

BD127_{—}16 protein was expressed in a COS cell expression system, and an expressed protein band of approximately 22 kDa was detected in membrane fractions using SDS polyacrylamide gel electrophoresis.

Clone “BL205_{—}14”

A polynucleotide of the present invention has been identified as clone “BL205_{—}14”. BL205_{—}14 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BL205_{—}14 is a full-length clone, including the entire coding sequence of a secreted protein (also referred to herein as “BL205_{—}14 protein”).

The nucleotide sequence of BL205_{—}14 as presently determined is reported in SEQ ID NO:187, and includes a poly(A) tail. What applicants presently believe to be the proper reading frame and the predicted amino acid sequence of the BL205_{—}14 protein corresponding to the foregoing nucleotide sequence is reported in SEQ ID NO:188.

The EcoRI/NotI restriction fragment obtainable from the deposit containing clone BL205_{—}14 should be approximately 1500 bp.

The nucleotide sequence disclosed herein for BL205_{—}14 was searched against the GenBank and GeneSeq nucleotide sequence databases using BLASTN/BLASTX and FASTA search protocols. BL205_{—}14 demonstrated at least some similarity with sequences identified as T08683 (EST06575 Homo sapiens cDNA clone HIBBI30 5′ end) and U55178 (Mus musculus TIL mRNA from progressing tumor site, clone NFB#3). Based upon sequence similarity, BL205_{—}14 proteins and each similar protein or peptide may share at least some activity.

Clone “H438_{—}1”

A polynucleotide of the present invention has been identified as clone “H438_{—}1”. H438_{—}1 was isolated from a human adult blood (peripheral blood mononuclear cells activated by treatment with phytohemagglutinin, phorbol myristate acetate, and mixed lymphocyte reaction) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. H438_{—}1 is a full-length clone, including the entire coding sequence of a secreted protein (also referred to herein as “H438_{—}1 protein”).

The nucleotide sequence of H438_{—}1 as presently determined is reported in SEQ ID NO:189, and includes a poly(A) tail. What applicants presently believe to be the proper reading frame and the predicted amino acid sequence of the H438_{—}1 protein corresponding to the foregoing nucleotide sequence is reported in SEQ ID NO:190.

The EcoRI/NotI restriction fragment obtainable from the deposit containing clone H438_{—}1 should be approximately 2100 bp.

The nucleotide sequence disclosed herein for H438_{—}1 was searched against the GenBank and GeneSeq nucleotide sequence databases using BLASTN/BLASTX and FASTA search protocols. H438_{—}1 demonstrated at least some similarity with sequences identified as H06234 (yl78e09.r1 Homo sapiens cDNA clone 44074 5′) and R56040 (yg91a04.s1 Homo sapiens cDNA clone 40669 3′). Based upon sequence similarity, H438_{—}1 proteins and each similar protein or peptide may share at least some activity. The TopPredII computer program predicts a potential transmembrane domain within the H438_{—}1 protein sequence, centered around amino acid 25 of SEQ ID NO:190.

Clone “AY421_{—}2”

A polynucleotide of the present invention has been identified as clone “AY421_{—}2”. AY421_{—}2 was isolated from a human adult retina cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. AY421_{—}2 is a full-length clone, including the entire coding sequence of a secreted protein (also referred to herein as “AY421_{—}2 protein”).

The nucleotide sequence of AY421_{—}2 as presently determined is reported in SEQ ID NO:191, and includes a poly(A) tail. What applicants presently believe to be the proper reading frame and the predicted amino acid sequence of the AY421_{—}2 protein corresponding to the foregoing nucleotide sequence is reported in SEQ ID NO:192.

Amino acids 186 to 198 of SEQ ID NO:192 are a predicted leader/signal sequence, with the predicted mature amino acid sequence beginning at amino acid 199. Due to the hydrophobic nature of the predicted leader/signal sequence, it is likely to act as a transmembrane domain should the predicted leader/signal sequence not be separated from the remainder of the AY421_{—}2 protein.

The EcoRI/NotI restriction fragment obtainable from the deposit containing clone AY421_{—}2 should be approximately 1500 bp.

The nucleotide sequence disclosed herein for AY421_{—}2 was searched against the GenBank and GeneSeq nucleotide sequence databases using BLASTN/BLASTX and FASTA search protocols. AY421_{—}2 demonstrated at least some similarity with sequences identified as AA016992 (ze33d02.r1 Soares retina N2b4HR Homo sapiens cDNA clone 360771 5′ similar to WP T06D8.5 CE02326 YER141W), H60299 (yr41b09.r1 Homo sapiens cDNA clone 207833 5′), L38643 (Saccharomyces cerevisiae cytochrome oxidase assembly), N25978 (yx88b04.s1 Homo sapiens cDNA clone 268783 3′), and R59851 (yh07a09.r1 Homo sapiens cDNA). The predicted amino acid sequence disclosed herein for AY421_{—}2 was searched against the GenPept and GeneSeq amino acid sequence databases using the BLASTX search protocol. The predicted AY421_{—}2 protein demonstrated at least some similarity to sequences identified as L38643 (YSCCOX15A_{—}1 cytochrome oxidase assembly factor [Saccharomyces cerevisiae]) and Z49130 (CET06D8_{—}5 T06D8.5 [Caenorhabditis elegans]). Based upon sequence similarity, AY421_{—}2 proteins and each similar protein or peptide may share at least some activity. The TopPredII computer program predicts six potential transmembrane domains within the AY421_{—}2 protein sequence, centered around amino acids 80, 193, 237, 274, 336, and 376 of SEQ ID NO:192, respectively.

Clone “BV278_{—}2”

A polynucleotide of the present invention has been identified as clone “BV278_{—}2”. BV278_{—}2 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BV278_{—}2 is a full-length clone, including the entire coding sequence of a secreted protein (also referred to herein as “BV278_{—}2 protein”).

The nucleotide sequence of BV278_{—}2 as presently determined is reported in SEQ ID NO:193, and includes a poly(A) tail. What applicants presently believe to be the proper reading frame and the predicted amino acid sequence of the BV278_{—}2 protein corresponding to the foregoing nucleotide sequence is reported in SEQ ID NO:194.

The EcoRI/NotI restriction fragment obtainable from the deposit containing clone BV278_{—}2 should be approximately 2300 bp.

The nucleotide sequence disclosed herein for BV278_{—}2 was searched against the GenBank and GeneSeq nucleotide sequence databases using BLASTN/BLASTX and FASTA search protocols. BV278_{—}2 demonstrated at least some similarity with sequences identified as AA359704 (EST68845 Fetal lung II Homo sapiens cDNA 5′ end). Based upon sequence similarity, BV278_{—}2 proteins and each similar protein or peptide may share at least some activity. The TopPredII computer program predicts a potential transmembrane domain within the BV278_{—}2 protein sequence centered around amino acid 19 of SEQ ID NO:194.

Clone “C544_{—}1”

A polynucleotide of the present invention has been identified as clone “C544_{—}1”. A cDNA clone was first isolated from a human adult blood (peripheral blood mononuclear cells activated by treatment with concanavalin A and phorbol myristate acetate) cDNA library using methods which are selective for cDNAs encoding secreted proteins (see U.S. Pat. No. 5,536,637). This cDNA clone was then used to isolate C544_{—}1 from a human adult blood (peripheral blood mononuclear cells activated by treatment with phytohemagglutinin, phorbol myristate acetate, and mixed lymphocyte reaction) cDNA library. C544_{—}1 is a full-length clone, including the entire coding sequence of a secreted protein (also referred to herein as “C544_{—}1 protein”).

The nucleotide sequence of C544_{—}1 as presently determined is reported in SEQ ID NO:195, and includes a poly(A) tail What applicants presently believe to be the proper reading frame and the predicted amino acid sequence of the C544_{—}1 protein corresponding to the foregoing nucleotide sequence is reported in SEQ ID NO:196.

The EcoRI/NotI restriction fragment obtainable from the deposit containing clone C544_{—}1 should be approximately 1250 bp.

The nucleotide sequence disclosed herein for C544_{—}1 was searched against the GenBank and GeneSeq nucleotide sequence databases using BLASTN/BLASTX and FASTA search protocols. C544_{—}1 demonstrated at least some similarity with sequences identified as AA029713 (ze95f03.s1 Soares fetal heart NbHH19W Homo sapiens cDNA clone 366749 3′), T53653 (ya98b10.s1 Homo sapiens cDNA clone 69691 3′), T85425 (yd76d07.r1 Homo sapiens cDNA clone 114157 5′), and Z97634 (Human DNA sequence *** SEQUENCING IN PROGRESS *** from clone 367G8; HTGS phase 1). Based upon sequence similarity, C544_{—}1 proteins and each similar protein or peptide may share at least some activity. The TopPredII computer program predicts seven potential transmembrane domains within the C544_{—}1 protein sequence, centered around amino acids 80, 110, 150, 170, 190, 220, and 260 of SEQ ID NO:196, respectively. C544_{—}1 protein also contains an EGF domain motif around amino acid 50 of SEQ ID NO:196. A single EGF domain has been shown to exist in other proteins including, but not limited to, human teratocarcinoma-derived growth factor 1, tissue plasminogen activator (TPA), and coagulation factors VU, IX, and X. A common feature of EGF repeats is that they are found in the extracellular domain of membrane-bound proteins or in proteins known to be secreted.

Clone “CC332_{—}33”.

A polynucleotide of the present invention has been identified as clone “CC332_{—}33”. CC332_{—}33 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CC332_{—}33 is a full-length clone, including the entire coding sequence of a secreted protein (also referred to herein as “CC332_{—}33 protein”).

The nucleotide sequence of CC332_{—}33 as presently determined is reported in SEQ ID NO:197, and includes a poly(A) tail. What applicants presently believe to be the proper reading frame and the predicted amino acid sequence of the CC332_{—}33 protein corresponding to the foregoing nucleotide sequence is reported in SEQ ID NO:198. Amino acids 150 to 162 of SEQ ID NO:198 are a predicted leader/signal sequence, with the predicted mature amino acid sequence beginning at amino acid 163. Due to the hydrophobic nature of the predicted leader/signal sequence, it is likely to act as a transmembrane domain should the predicted leader/signal sequence not be separated from the remainder of the CC332_{—}33 protein.

The EcoRI/NotI restriction fragment obtainable from the deposit containing clone CC332_{—}33 should be approximately 4400 bp.

The nucleotide sequence disclosed herein for CC332_{—}33 was searched against the GenBank and GeneSeq nucleotide sequence databases using BLASTN/BLASTX and FASTA search protocols. CC332_{—}33 demonstrated at least some similarity with sequences identified as N63467 (yy61d06.s1 Homo sapiens cDNA clone 278027 3′). Based upon sequence similarity, CC332_{—}33 proteins and each similar protein or peptide may share at least some activity. The TopPredII computer program predicts a potential transmembrane domain within the CC332_{—}33 protein sequence, centered around amino acid 10 of SEQ ID NO:198, which could also function as a secretory signal sequence.

Clone “CC365_{—}40”

A polynucleotide of the present invention has been identified as clone “CC365_{—}40”. CC365_{—}40 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CC365_{—}40 is a full-length clone, including the entire coding sequence of a secreted protein (also referred to herein as “CC365_{—}40 protein”).

The nucleotide sequence of CC365_{—}40 as presently determined is reported in SEQ ID NO:199, and includes a poly(A) tail. What applicants presently believe to be the proper reading frame and the predicted amino acid sequence of the CC365_{—}40 protein corresponding to the foregoing nucleotide sequence is reported in SEQ ID NO:200. Amino acids 29 to 41 of SEQ ID NO:200 are a predicted leader/signal sequence, with the predicted mature amino acid sequence beginning at amino acid 42. Due to the hydrophobic nature of the predicted leader/signal sequence, it is likely to act as a transmembrane domain should the predicted leader/signal sequence not be separated from the remainder of the CC365_{—}40 protein.

The EcoRI/NotI restriction fragment obtainable from the deposit containing clone CC365_{—}40 should be approximately 1380 bp.

The nucleotide sequence disclosed herein for CC365_{—}40 was searched against the GenBank and GeneSeq nucleotide sequence databases using BLASTN/BLASTX and FASTA search protocols. CC365_{—}40 demonstrated at least some similarity with sequences identified as H33410 (EST109372 Rattus sp. cDNA 5′ end) and W21840 (58b3 Human retina cDNA Tsp509]-cleaved sublibrary Homo sapiens cDNA). The predicted amino acid sequence disclosed herein for CC365_{—}40 was searched against the GenPept and GeneSeq amino acid sequence databases using the BLASTX search protocol. The predicted CC365_{—}40 protein demonstrated at least some similarity to sequences identified as U13625 (cytochrome b [Pezoporus wallicus]). Based upon sequence similarity, CC365_{—}40 proteins and each similar protein or peptide may share at least some activity. The TopPredII computer program predicts a potential transmembrane domain within the CC365_{—}40 protein sequence centered around amino acid 42 of SEQ ID NO:200.

Clone “CG68_{—}4”

A polynucleotide of the present invention has been identified as clone “CG68_{—}4”. CG68_{—}4 was isolated from a human adult testes cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CG68_{—}4 is a full-length clone, including the entire coding sequence of a secreted protein (also referred to herein as “CG68_{—}4 protein”).

The nucleotide sequence of CG68_{—}4 as presently determined is reported in SEQ ID NO:201, and includes a poly(A) tail. What applicants presently believe to be the proper reading frame and the predicted amino acid sequence of the CG68_{—}4 protein corresponding to the foregoing nucleotide sequence is reported in SEQ ID NO:202. Amino acids 2 to 14 of SEQ ID NO:202 are a predicted leader/signal sequence, with the predicted mature amino acid sequence beginning at amino acid 15. Due to the hydrophobic nature of the predicted leader/signal sequence, it is likely to act as a transmembrane domain should the predicted leader/signal sequence not be separated from the remainder of the CG68_{—}4 protein.

The EcoRI/NotI restriction fragment obtainable from the deposit containing clone CG68_{—}4 should be approximately 1080 bp.

The predicted amino acid sequence disclosed herein for CG68_{—}4 was searched against the GenPept and GeneSeq amino acid sequence databases using the BLASTX search protocol. The predicted CG68_{—}4 protein demonstrated at least some similarity to sequences identified as M29854 (interleukin 4 receptor (IL-4) precursor). Based upon sequence similarity, CG68_{—}4 proteins and each similar protein or peptide may share at least some activity. The TopPredII computer program predicts an additional potential transmembrane domains within the CG68_{—}4 protein sequence centered around amino acid 190 of SEQ ID NO:202. The nucleotide sequence of CG68_{—}4 indicates that it may contain one ore more L1 repeats.

CG68_{—}4 protein was expressed in a COS cell expression system, and an expressed protein band of approximately 19 kDa was detected in conditioned medium using SDS polyacrylamide gel electrophoresis.

Clone “D329_{—}1”

A polynucleotide of the present invention has been identified as clone “D329_{—}1”. A cDNA clone was first isolated from a human adult blood (peripheral blood mononuclear cells activated by treatment with concanavalin A and phorbol myristate acetate) cDNA library using methods which are selective for cDNAs encoding secreted proteins (see U.S. Pat. No. 5,536,637). This cDNA clone was then used to isolate D329_{—}1 from a human adult blood (peripheral blood mononuclear cells activated by treatment with phytohemagglutinin, phorbol myristate acetate, and mixed lymphocyte reaction) cDNA library. D329_{—}1 is a full-length clone, including the entire coding sequence of a secreted protein (also referred to herein as “D329_{—}1 protein”).

The nucleotide sequence of D329_{—}1 as presently determined is reported in SEQ ID NO:203, and includes a poly(A) tail. What applicants presently believe to be the proper reading frame and the predicted amino acid sequence of the D329_{—}1 protein corresponding to the foregoing nucleotide sequence is reported in SEQ ID NO:204. Amino acids 69 to 81 of SEQ ID NO:204 are a predicted leader/signal sequence, with the predicted mature amino acid sequence beginning at amino acid 82. Due to the hydrophobic nature of the predicted leader/signal sequence, it is likely to act as a transmembrane domain should the predicted leader/signal sequence not be separated from the remainder of the D329_{—}1 protein.

The EcoRI/NotI restriction fragment obtainable from the deposit containing clone D329_{—}1 should be approximately 3100 bp.

The nucleotide sequence disclosed herein for D329_{—}1 was searched against the GenBank and GeneSeq nucleotide sequence databases using BLASTN/BLASTX and FASTA search protocols. D329_{—}1 demonstrated at least some similarity with sequences identified as W46599 (zc32g12.r1 Soares senescent fibroblasts NbHSF Homo sapiens cDNA). The predicted amino acid sequence disclosed herein for D329_{—}1 was searched against the GenPept and GeneSeq amino acid sequence databases using the BLASTX search protocol. The predicted D329_{—}1 protein demonstrated at least some similarity to sequences identified as U40941 (coded for by C. elegans cDNA CEESB82F). Based upon sequence similarity, D329_{—}1 proteins and each similar protein or peptide may share at least some activity.

Clone “H698_{—}3”

A polynucleotide of the present invention has been identified as clone “H698_{—}3”. H698_{—}3 was isolated from a human adult blood (peripheral blood mononuclear cells activated by treatment with phytohemagglutinin, phorbol myristate acetate, and mixed lymphocyte reaction) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. H698_{—}3 is a full-length clone, including the entire coding sequence of a protein (also referred to herein as “H698_{—}3 protein”).

The nucleotide sequence of the 5′ portion of H698_{—}3 as presently determined is reported in SEQ ID NO:205. An additional internal nucleotide sequence from H698_{—}3 as presently determined is reported in SEQ ID NO:206. What applicants believe is the proper reading frame and the predicted amino acid sequence encoded by such internal sequence is reported in SEQ ID NO:207. Additional nucleotide sequence from the 3′ portion of H698_{—}3, including a poly(A) tail, is reported in SEQ ID NO:208.

The EcoRI/NotI restriction fragment obtainable from the deposit containing clone H698_{—}3 should be approximately 1400 bp.

The nucleotide sequence disclosed herein for H698_{—}3 was searched against the GenBank and GeneSeq nucleotide sequence databases using BLASTN/BLASTX and FASTA search protocols. H698_{—}3 demonstrated at least some similarity with sequences identified as N69846 (za67e04.s1 Homo sapiens cDNA clone 297630 3′ similar to SW RT05_YEAST P33759 PROBABLE MITOCHONDRIAL 40S RIBOSOMAL PROTEIN S5), W36396 (mb75e09.r1 Soares mouse p3NMF19.5 Mus musculus cDNA clone 335272 5′), and W81295 (zd85h08.r1 Soares fetal heart NbHH19W Homo sapiens cDNA clone 347487 5′). The predicted amino acid sequence disclosed herein for H698_{—}3 was searched against the GenPept and GeneSeq amino acid sequence databases using the BLASTX search protocol. The predicted H698_{—}3 protein demonstrated at least some similarity to sequences identified as L20296 (homology with a procaryotic 30S ribosomal protein S5 [Saccharomyces cerevisiae]) and Z69727 (unknown [Schizosaccharomyces pombe]). Based upon sequence similarity, H698_{—}3 proteins and each similar protein or peptide may share at least some activity. H698_{—}3 shares sequence similarity with several other ribosomal proteins and with polynucleotide sequences that encode ribosomal proteins or proteins similar to ribosomal proteins.

Clone “H963_{—}20”

A polynucleotide of the present invention has been identified as clone “H963_{—}20”. H963_{—}20 was isolated from a human adult blood (peripheral blood mononuclear cells activated by treatment with phytohemagglutinin, phorbol myristate acetate, and mixed lymphocyte reaction) cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. H963_{—}20 is a full-length clone, including the entire coding sequence of a secreted protein (also referred to herein as “H963_{—}20 protein”).

The nucleotide sequence of H963_{—}20 as presently determined is reported in SEQ ID NO:209, and includes a poly(A) tail. What applicants presently believe to be the proper reading frame and the predicted amino acid sequence of the H963_{—}20 protein corresponding to the foregoing nucleotide sequence is reported in SEQ ID NO:210. Amino acids 23 to 35 of SEQ ID NO:210 are a predicted leader/signal sequence, with the predicted mature amino acid sequence beginning at amino acid 36. Due to the hydrophobic nature of the predicted leader/signal sequence, it is likely to act as a transmembrane domain should the predicted leader/signal sequence not be separated from the remainder of the H963_{—}20 protein.

The EcoRI/NotI restriction fragment obtainable from the deposit containing clone H963_{—}20 should be approximately 1240 bp.

The nucleotide sequence disclosed herein for H963_{—}20 was searched against the GenBank and GeneSeq nucleotide sequence databases using BLASTN/BLASTX and FASTA search protocols. H963_{—}20 demonstrated at least some similarity with sequences identified as AA184698 (mt58f09.r1 Soares 2NbMT Mus musculus cDNA clone 634121 5′ similar to TR G285995 G285995 ORF, CMPLETE CDS.). The predicted amino acid sequence disclosed herein for H963_{—}20 was searched against the GenPept and GeneSeq amino acid sequence databases using the BLASTX search protocol. The predicted H963_{—}20 protein demonstrated at least some similarity to sequences identified as D13626 (KIAA0001 [Homo sapiens]), U33447 (putative G-protein-coupled receptor [Homo sapiens]), and WO4246 (Human G-protein coupled receptor GPR3). Based upon sequence similarity, H963_{—}20 proteins and each similar protein or peptide may share at least some activity. Based upon sequence similarity, H963_{—}20 proteins and each similar protein or peptide may share at least some activity. The TopPredII computer program predicts seven potential transmembrane domains within the H963_{—}20 protein sequence, centered around amino acids 36, 60, 100, 140, 190, 230, and 280 of SEQ ID NO:210, respectively.

Clone “BD372_{—}5”

A polynucleotide of the present invention has been identified as clone “BD372_{—}5”. BD372_{—}5 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BD372_{—}5 is a full-length clone, including the entire coding sequence of a secreted protein (also referred to herein as “BD372_{—}5 protein”).

The nucleotide sequence of BD372_{—}5 as presently determined is reported in SEQ ID NO:211, and includes a poly(A) tail. What applicants presently believe to be the proper reading frame and the predicted amino acid sequence of the BD372_{—}5 protein corresponding to the foregoing nucleotide sequence is reported in SEQ ID NO:212. Amino acids 12 to 24 of SEQ ID NO:212 are a predicted leader/signal sequence, with the predicted mature amino acid sequence beginning at amino acid 25. Due to the hydrophobic nature of the predicted leader/signal sequence, it is likely to act as a transmembrane domain should the predicted leader/signal sequence not be separated from the remainder of the BD372_{—}5 protein.

The EcoRI/NotI restriction fragment obtainable from the deposit containing clone BD372_{—}5 should be approximately 2300 bp.

The nucleotide sequence disclosed herein for BD372_{—}5 was searched against the GenBank and GeneSeq nucleotide sequence databases using BLASTN/BLASTX and FASTA search protocols. BD372_{—}5 demonstrated at least some similarity with sequences identified as R39276 (yc90f12.s1 Homo sapiens cDNA clone 23278 3′) and T07647 (EST05537 Homo sapiens cDNA clone HFBEM26). Based upon sequence similarity, BD372_{—}5 proteins and each similar protein or peptide may share at least some activity. The nucleotide sequence of BD372_{—}5 indicates that it may contain one or more of the following repetitive elements: Alu, SVA.

Clone “BR533_{—}4”

A polynucleotide of the present invention has been identified as clone “BR533_{—}4”. BR533_{—}4 was isolated from a human fetal kidney cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. BR533_{—}4 is a full-length clone, including the entire coding sequence of a secreted protein (also referred to herein as “BR533_{—}4 protein”).

The nucleotide sequence of BR533_{—}4 as presently determined is reported in SEQ ID NO:213, and includes a poly(A) tail. What applicants presently believe to be the proper reading frame and the predicted amino acid sequence of the BR533_{—}4 protein corresponding to the foregoing nucleotide sequence is reported in SEQ ID NO:214. Amino acids 6 to 18 of SEQ ID NO:214 are a predicted leader/signal sequence, with the predicted mature amino acid sequence beginning at amino acid 19. Due to the hydrophobic nature of the predicted leader/signal sequence, it is likely to act as a transmembrane domain should the predicted leader/signal sequence not be separated from the remainder of the BR533_{—}4 protein.

The EcoRI/NotI restriction fragment obtainable from the deposit containing clone BR533_{—}4 should be approximately 2850 bp.

The nucleotide sequence disclosed herein for BR533_{—}4 was searched against the GenBank and GeneSeq nucleotide sequence databases using BLASTN/BLASTX and FASTA search protocols. BR533_{—}4 demonstrated at least some similarity with sequences identified as AA043044 (zk53h08.r1 Soares pregnant uterus NbHPU Homo sapiens cDNA clone 486591 5′), AA160999 (zq41d11.r1 Stratagene hNT neuron (#937233) Homo sapiens cDNA clone 632277 5′ similar to TR G854332 G854332 SEMAPHORIN E), AB000220 (Human mRNA for semaphorin E, complete cds.), F14663 (S. scrofa mRNA; expressed sequence tag (5′; clone c7b01)), N38844 (yy80d10.s1 Homo sapiens cDNA clone 279859 3′), Q87442 (Human semaphorin III cDNA), and X85994 (M. musculus mRNA for semaphorin E). The predicted amino acid sequence disclosed herein for BR533_{—}4 was searched against the GenPept and GeneSeq amino acid sequence databases using the BLASTX search protocol. The predicted BR533_{—}4 protein demonstrated at least some similarity to sequences identified as AB00022 (1777307 (Accession #: AB000220) semaphorin E [Homo sapiens]), R71380 (R71380 (Accession #: R71380) Human semaphorin III protein), U38276 (semaphorin III family homolog [Homo sapiens]), and X85994 (MMRNASEME_{—}1 semaphorin E [Mus musculus]). Semaphorins are a diverse family of molecules that may provide local signals to specify territories that are not accessible to growing axons. Members of the semaphorin gene family appear to act as growth cone guidance signals in both invertebrates and vertebrates. Based upon sequence similarity, BR533_{—}4 proteins and each similar protein or peptide may share at least some activity. The TopPredII computer program predicts two potential transmembrane domains within the BR533_{—}4 protein sequence, one centered around amino acid 320 and another around amino acid 120 of SEQ ID NO:214.

BR533_{—}4 protein was expressed in a COS cell expression system, and an expressed protein band of approximately 110 kDa was detected in conditioned medium and membrane fractions using SDS polyacrylamide gel electrophoresis.

Clone “CC288_{—}9”

A polynucleotide of the present invention has been identified as clone “CC288_{—}9”. CC288_{—}9 was isolated from a human adult brain cDNA library using methods which are selective for cDNAs encoding secreted proteins (see U.S. Pat. No. 5,536,637), or was identified as encoding a secreted or transmembrane protein on the basis of computer analysis of the amino acid sequence of the encoded protein. CC288_{—}9 is a full-length clone, including the entire coding sequence of a secreted protein (also referred to herein as “CC288_{—}9 protein”).

The nucleotide sequence of CC288_{—}9 as presently determined is reported in SEQ ID NO:215, and includes a poly(A) tail. What applicants presently believe to be the proper reading frame and the predicted amino acid sequence of the CC288_{—}9 protein corresponding to the foregoing nucleotide sequence is reported in SEQ ID NO:216.

The EcoRI/NotI restriction fragment obtainable from the deposit containing clone CC288_{—}9 should be approximately 650 bp.

The nucleotide sequence disclosed herein for CC288_{—}9 was searched against the GenBank and GeneSeq nucleotide sequence databases using BLASTN/BLASTX and FASTA search protocols. CC288_{—}9 demonstrated at least some similarity with sequences identified as AA099962 (zl79d05.r1 Stratagene colon (#937204) Homo sapiens cDNA clone 510825 5′) and AA272295 (vb61d03.r1 Barstead mouse pooled organs MPLRB4 Mus musculus cDNA clone 761477 5′). Based upon sequence similarity, CC288_{—}9 proteins and each similar protein or peptide may share at least some activity. The TopPredII computer program predicts two potential transmembrane domains within the CC288_{—}9 protein sequence, one centered around amino acid 20 and another around amino acid 80 of SEQ ID NO:216.

Deposit of Clones

Clone B18_{—}11 was deposited on Jul. 6, 1995 with the ATCC (American Type Culture Collection, 10801 University Boulevard, Manassas, Va. 20110-2209 U.S.A.) as an original deposit under the Budapest Treaty and was given the accession number 69868.

Clone H174 was deposited on Aug. 11, 1995 with the ATCC (American Type Culture Collection, 10801 University Boulevard, Manassas, Va. 20110-2209 U.S.A.) as an original deposit under the Budapest Treaty and was given the accession number 69882.

Clone J5_{—}3 was deposited on Aug. 11, 1995 with the ATCC (American Type Culture Collection, 10801 University Boulevard, Manassas, Va. 20110-2209 U.S.A.) as an original deposit under the Budapest Treaty and was given the accession number 69885.

Clone J422_{—}1 was deposited on Aug. 11, 1995 with the ATCC (American Type Culture Collection, 10801 University Boulevard, Manassas, Va. 20110-2209 U.S.A.) as an original deposit under the Budapest Treaty and was given the accession number 69984.

Clone L105_{—}74 was deposited on Aug. 11, 1995 with the ATCC (American Type Culture Collection, 10801 University Boulevard, Manassas, Va. 20110-2209 U.S.A.) as an original deposit under the Budapest Treaty and was given the accession number 69883.

Clone B121_{—}1 was deposited on Apr. 4, 1996 with the ATCC (American Type Culture Collection, 10801 University Boulevard, Manassas, Va. 20110-2209 U.S.A.) as an original deposit under the Budapest Treaty and was given the accession number 98019.

Clone B196_{—}122 was deposited on Apr. 4, 1996 with the ATCC (American Type Culture Collection, 10801 University Boulevard, Manassas, Va. 20110-2209 U.S.A.) as an original deposit under the Budapest Treaty and was given the accession number 98021.

Clone D157_{—}4 was deposited on Apr. 4, 1996 with the ATCC (American Type Culture Collection, 10801 University Boulevard, Manassas, Va. 20110-2209 U.S.A.) as an original deposit under the Budapest Treaty and was given the accession number 98020.

Clones B219_{—}2, G52_{—}24, G86_{—}2, H83_{—}22, H298_{—}23, H849_{—}24, H905_{—}107, H1075_{—}1, J59_{—}41, J143_{—}1, J218_{—}15, M8_{—}2, M97_{—}2, O238_{—}1, and S185_{—}2 were deposited on Apr. 19, 1996 with the ATCC (American Type Culture Collection, 10801 University Boulevard, Manassas, Va. 20110-2209 U.S.A.) as an original deposit under the Budapest Treaty and were given the accession number 98028, from which each clone comprising a particular polynucleotide is obtainable.

Clones AJ147_{—}1, AM262_{—}11, AR28_{—}1, AS86_{—}1, AS162_{—}1, AS264_{—}3, AS268_{—}1, AS301_{—}2, AU105_{—}14, AU139_{—}2, AZ302_{—}1, D147_{—}17, and O75_{—}9 were deposited on Jun. 6, 1996 with the ATCC (American Type Culture Collection, 10801 University Boulevard, Manassas, Va. 20110-2209 U.S.A.) as an original deposit under the Budapest Treaty and were given the accession number 98076, from which each clone comprising a particular polynucleotide is obtainable.

Clones AS152_{—}1, AS167_{—}3, AU47_{—}8, AU122_{—}1, BF208_{—}1, BG513_{—}19, BG556_{—}8, C195_{—}1, and O276_{—}16 were deposited on Jun. 15, 1996 with the ATCC (American Type Culture Collection, 10801 University Boulevard, Manassas. Va. 20110-2209 U.S.A.) as an original deposit under the Budapest Treaty and were given the accession number 98079, from which each clone comprising a particular polynucleotide is obtainable. An additional isolate of AS152_{—}1 (identified as “AS152_{—}2”) was deposited on Sep. 26, 1996 with the ATCC (American Type Culture Collection, 10801 University Boulevard, Manassas, Va. 20110-2209 U.S.A.) as an original deposit under the Budapest Treaty and was given the accession number 98181. An additional isolate of C195_{—}1 (identified as “C195_{—}4”) was deposited on Sep. 26, 1996 with the ATCC (American Type Culture Collection, 10801 University Boulevard, Manassas, Va. 20110-2209 U.S.A.) as an original deposit under the Budapest Treaty and was given the accession number 98182.

Clones AC41_{—}1, AC222_{—}1, AJ143_{—}1, AJ168_{—}4, AK684_{—}1, AS209_{—}1, AX92_{—}3, BF245_{—}1, BG33_{—}7, J317_{—}1 and O289_{—}1, along with AX56_{—}8 and BM46_{—}3 (additional isolates of clones AX56_{—}28 and BM46_{—}10, respectively), were deposited on Jul. 9, 1996 with the ATCC (American Type Culture Collection, 10801 University Boulevard, Manassas, Va. 20110-2209 U.S.A.) as an original deposit under the Budapest Treaty and were given the accession number 98101, from which each clone comprising a particular polynucleotide is obtainable. AX56_{—}28 was deposited on Sep. 26, 1996 with the ATCC (American Type Culture Collection, 10801 University Boulevard, Manassas, Va. 20110-2209 U.S.A.) as an original deposit under the Budapest Treaty and was given the accession number 98180; BM46_{—}10 was deposited on Aug. 23, 1996 with the ATCC (American Type Culture Collection, 10801 University Boulevard, Manassas, Va. 20110-2209 U.S.A.) as an original deposit under the Budapest Treaty and was given the accession number 98152.

Clones AJ26_{—}3, AJ172_{—}2, AP224_{—}2, BL89_{—}10, BL341_{—}4, BV239_{—}2, CC25_{—}16, CC397_{—}11, D305_{—}2, G55_{—}1, K39_{—}7, K330_{—}3, K363_{—}3, K446_{—}3, K464_{—}4, K483_{—}1, and L69_{—}3 were deposited on Jul. 25, 1996 with the ATCC (American Type Culture Collection, 10801 University Boulevard, Manassas, Va. 20110-2209 U.S.A.) as an original deposit under the Budapest Treaty and were given the accession number 98115, from which each clone comprising a particular polynucleotide is obtainable. Clones K39_{—}7, K330_{—}3, K363_{—}3, K446_{—}3, K464_{—}4, and L69_{—}3 were referred to as K39_{—}2, K330_{—}2, K363_{—}2, K446_{—}2, K464_{—}3, and L69_{—}2, respectively, when the Jul. 25, 1996 deposit was made. An additional isolate of each of clones BL89_{—}10, BV239_{—}2, CC25_{—}16, and CC397_{—}11 (namely isolates BL89_{—}13, BV239_{—3}, CC25_{—17}, and CC397_{—19}, respectively) were deposited with the ATCC (American Type Culture Collection, 10801 University Boulevard, Manassas, Va. 20110-2209 U.S.A.) on Aug. 23, 1996 and were given the accession number 98153, from which each clone comprising a particular polynucleotide is obtainable.

Clones BG511_{—}30, BL15_{—}1, K289_{—}4, and K322_{—}4 were deposited on Aug. 1, 1996 with the ATCC (American Type Culture Collection, 10801 University Boulevard, Manassas, Va. 20110-2209 U.S.A.) as an original deposit under the Budapest Treaty and were given the accession number 98117, from which each clone comprising a particular polynucleotide is obtainable. Clones K289_{—}4 and K322_{—}4 were referred to as K289_{—}3 and K322_{—}2, respectively, when the Aug. 1, 1996 deposit was made. Clone BL15_{—}12, an additional isolate of clone BL15_{—}1, was deposited on Aug. 23, 1996 with the ATCC (American Type Culture Collection, 10801 University Boulevard, Manassas, Va. 20110-2209 U.S.A.) as an original deposit under the Budapest Treaty and was given the accession number 98154.

Clones AM349_{—}1, AR310_{—}2, AS186_{—}2, AY160_{—}1, BD127_{—}1, BL205_{—}7, and H438_{—}1 were deposited on Aug. 14, 1996 with the ATCC (American Type Culture Collection, 10801 University Boulevard, Manassas, Va. 20110-2209 U.S.A.) as an original deposit under the Budapest Treaty and were given the accession number 98140, from which each clone comprising a particular polynucleotide is obtainable. An additional isolate of each of clones AM349_{—}1, AR3102, AS186_{—}2, AY160_{—}1, BD127_{—}11, BL205_{—}7 (namely AM349_{—}2, AR310_{—}3, AS186_{—}3, AY160_{—}2, BD127_{—}16, and BL205_{—}14, respectively) was deposited on Aug. 23, 1996 with the ATCC (American Type Culture Collection, 10801 University Boulevard, Manassas, Va. 20110-2209 U.S.A.) as an original deposit under the Budapest Treaty and were given the accession number 98155, from which each clone comprising a particular polynucleotide is obtainable.

Clones AY421_{—}2, BV278_{—}2, C544_{—}1, CC332_{—}33, CC365_{—}40, CG68_{—}4, D329_{—}1, H698_{—}3, and H963_{—}20 were deposited on Aug. 22, 1996 with the ATCC (American Type Culture Collection, 10801 University Boulevard, Manassas, Va. 20110-2209 U.S.A.) as an original deposit under the Budapest Treaty and were given the accession number 98145, from which each clone comprising a particular polynucleotide is obtainable.

Clones BD372_{—}5, BR533_{—}4, and CC288_{—}9 were deposited on Aug. 22, 1996 with the American Type Culture Collection (10801 University Boulevard, Manassas, Va. 20110-2209 U.S.A.) as an original deposit under the Budapest Treaty and were given the accession number 98146, from which each clone comprising a particular polynucleotide is obtainable.

All restrictions on the availability to the public of the deposited material will be irrevocably removed upon the granting of the patent, except for the requirements specified in 37 C.F.R. § 1.808(b), and the term of the deposit will comply with 37 C.F.R. § 1.806.

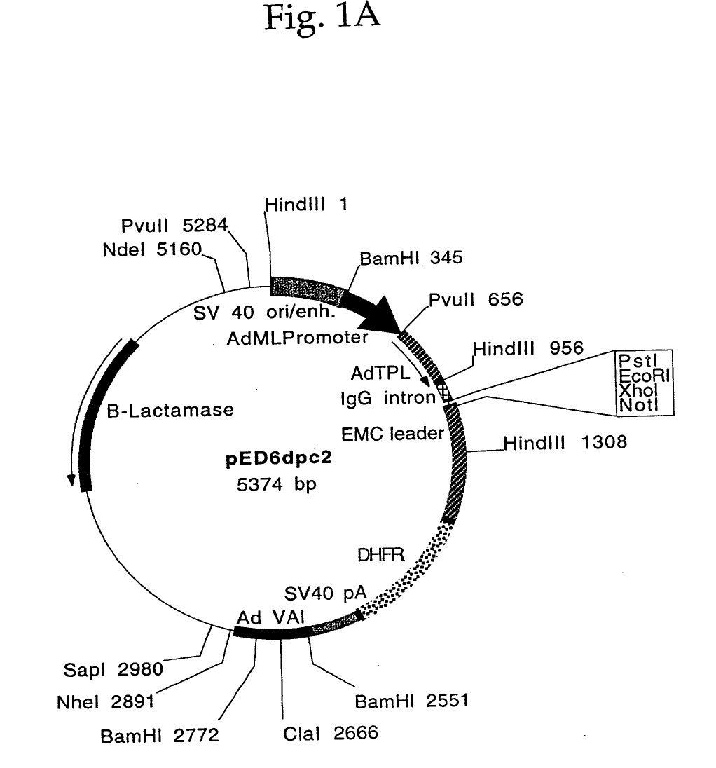

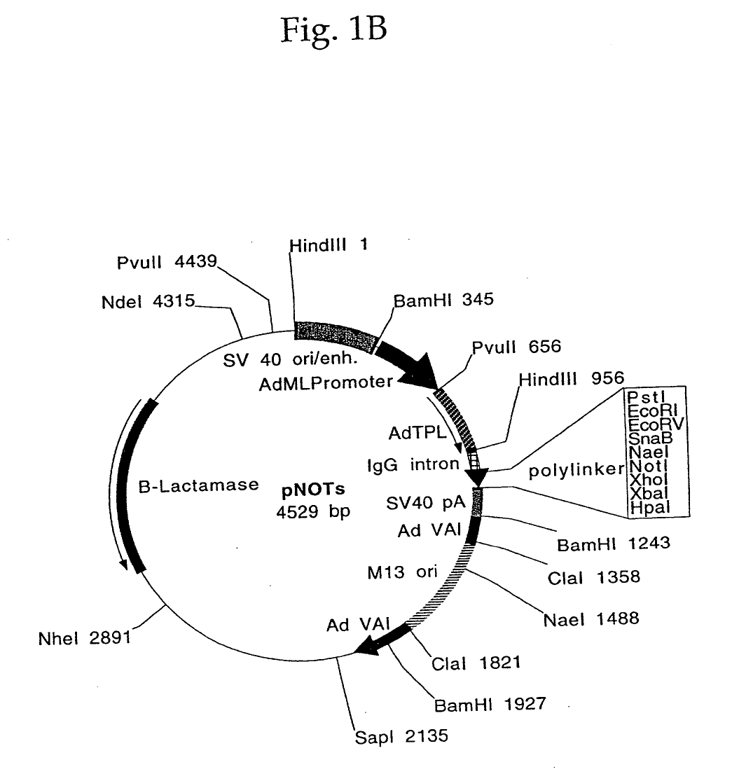

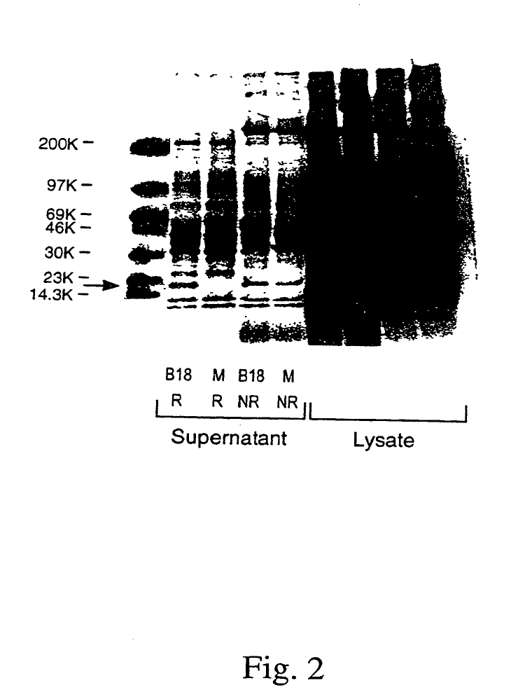

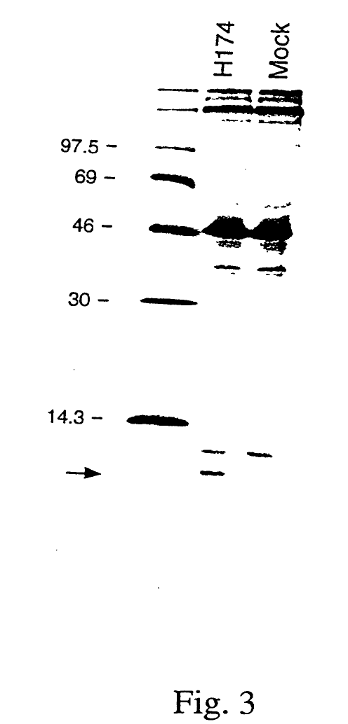

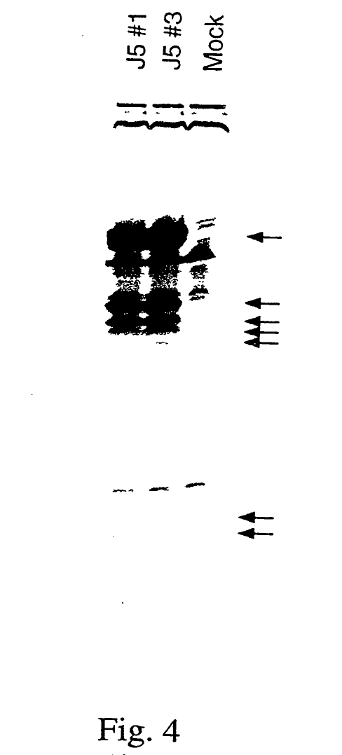

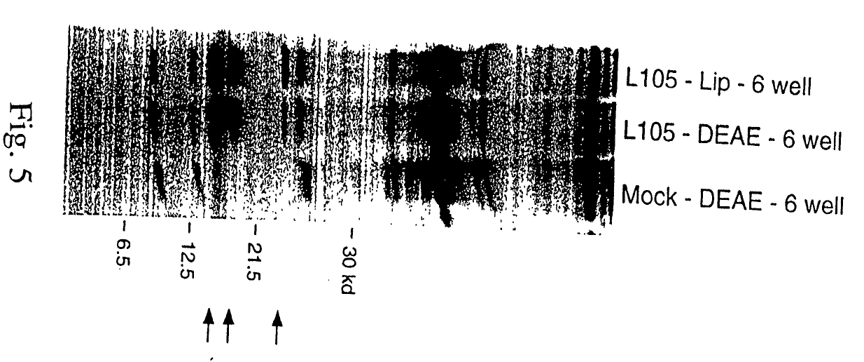

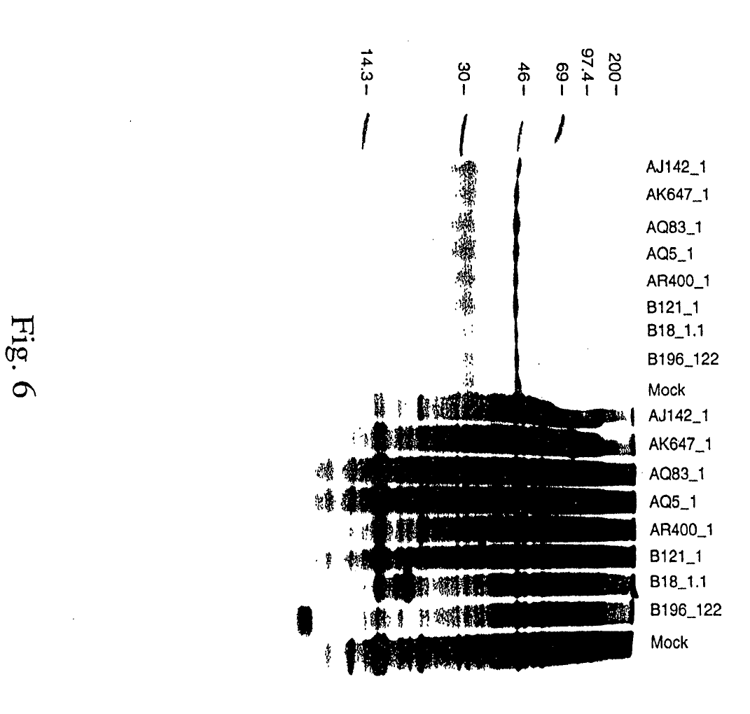

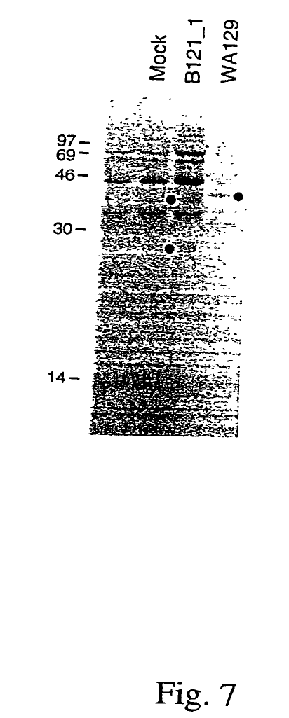

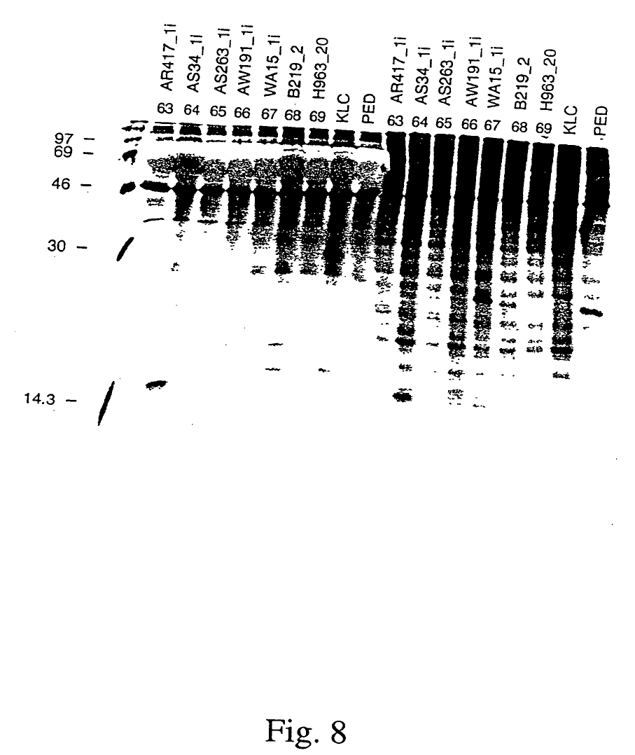

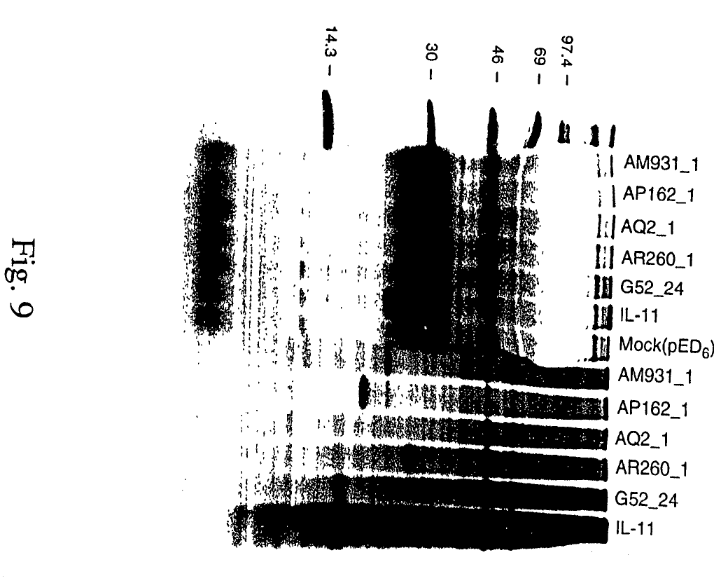

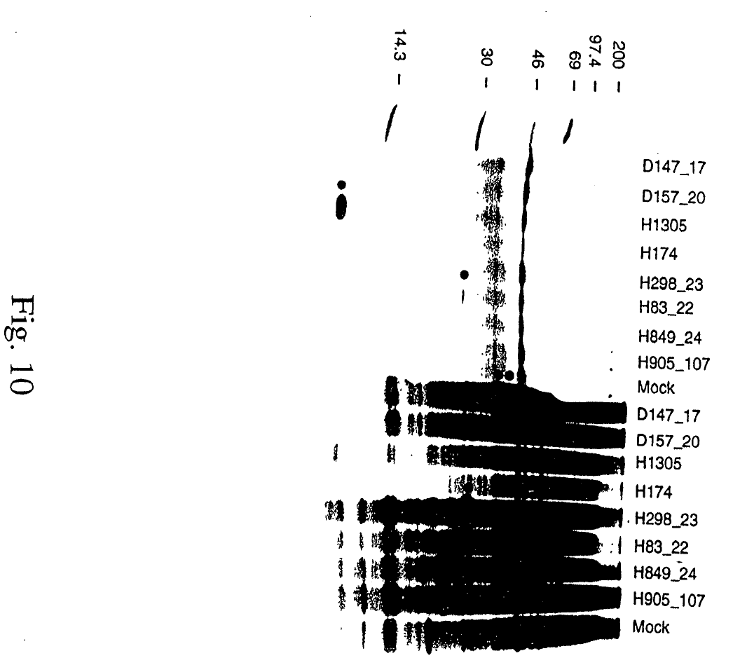

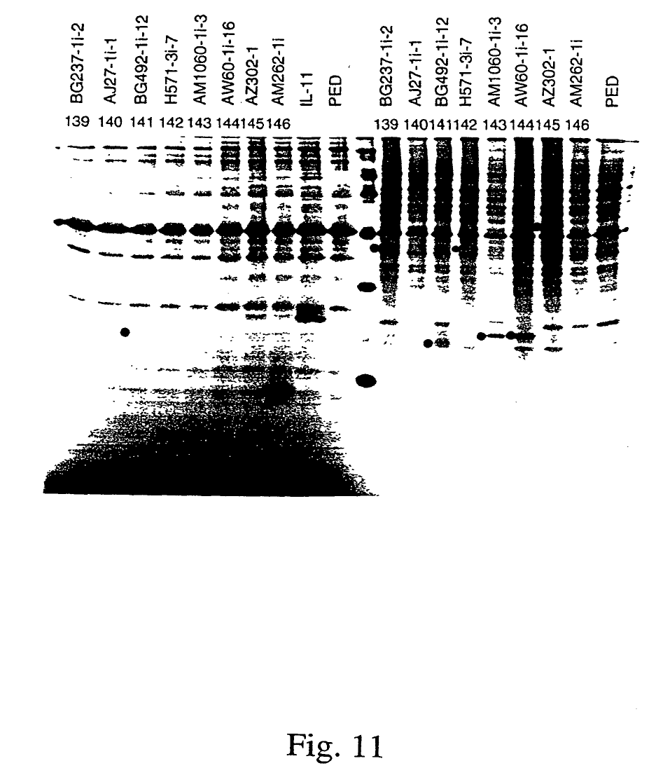

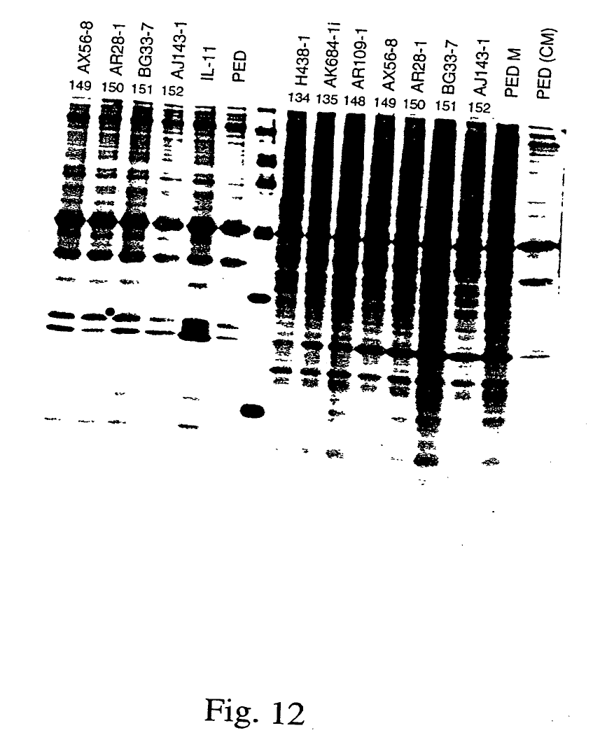

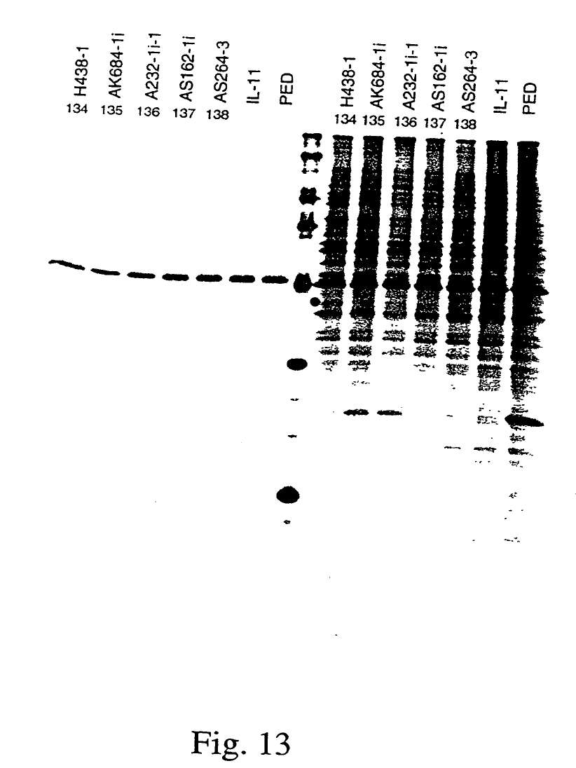

Each clone has been transfected into separate bacterial cells (E. coli) in this composite deposit. Each clone can be removed from the vector in which it was deposited by performing an EcoRI/NotI digestion (5′ site, EcoRI; 3′ site, NotI) to produce the appropriate fragment for such clone. Each clone (except for B18_{—}11, O276_{—}16, and O289_{—}1—see below) was deposited in either the pED6 or pNOTs vector depicted in FIGS. 1A and 1B, respectively. The pED6dpc2 vector (“pED6”) was derived from pED6dpc1 by insertion of a new polylinker to facilitate cDNA cloning (Kaufman et al., 1991, Nucleic Acids Res. 19: 4485-4490); the pNOTs vector was derived from pMT2 (Kaufman et al., 1989, Mol. Cell. Biol. 9: 946-958) by deletion of the DHFR sequences, insertion of a new polylinker, and insertion of the M13 origin of replication in the ClaI site. In some instances, the deposited clone can become “flipped” (i.e., in the reverse orientation) in the deposited isolate. In such instances, the cDNA insert can still be isolated by digestion with EcoRI and NotI. However, NotI will then produce the 5′ site and EcoRI will produce the 3′ site for placement of the cDNA in proper orientation for expression in a suitable vector. The proteins encoded by the cDNA clones may also be expressed from the vectors in which the clones were deposited.

Clone B18_{—}11 was deposited in the COS expression vector pMV2 (see WO 97/04097). Clones O276_{—}16 and O289_{—}1 were deposited in the Bluescript vector, in which instance the cDNA insert can be removed from the vector by digestion with XhoI (5′ site) and NotI (3′ site).

Bacterial cells containing a particular clone can be obtained from the composite deposits as follows:

An oligonucleotide probe or probes should be designed to the sequence that is known for that particular clone. This sequence can be derived from the sequences provided herein, or from a combination of those sequences. The sequence of an oligonucleotide probe that was used to isolate or to sequence each full-length clone is identified below, and should be most reliable in isolating the clone of interest.

| Clone | Probe Sequence |
|---|---|
| B18_11 | SEQ ID NO: 217, SEQ ID NO: 218, |
| | SEQ ID NO: 219 |
| D157_4 | SEQ ID NO: 220 |
| B219_2 | SEQ ID NO: 221 |
| G52_24 | SEQ ID NO: 222 |
| G86_2 | SEQ ID NO: 223 |
| H83_22 | SEQ ID NO: 224 |
| H298_23 | SEQ ID NO: 225 |
| H849_24 | SEQ ID NO: 226 |
| H905_107 | SEQ ID NO: 227 |
| H1075_1 | SEQ ID NO: 228 |
| J59_41 | SEQ ID NO: 229 |
| J143_1 | SEQ ID NO: 230 |
| J218_15 | SEQ ID NO: 231 |
| M8_2 | SEQ ID NO: 232 |
| M97_2 | SEQ ID NO: 233 |
| O238_1 | SEQ ID NO: 234 |
| S185_2 | SEQ ID NO: 235 |
| AJ147_1 | SEQ ID NO: 236 |
| AM262_11 | SEQ ID NO: 237 |
| AR28_1 | SEQ ID NO: 238 |
| AS86_1 | SEQ ID NO: 239 |
| AS162_1 | SEQ ID NO: 240 |
| AS264_3 | SEQ ID NO: 241 |
| AS268_1 | SEQ ID NO: 242 |
| AS301_2 | SEQ ID NO: 243 |
| AU105_14 | SEQ ID NO: 244 |
| AU139_2 | SEQ ID NO: 245 |
| AZ302_1 | SEQ ID NO: 246 |
| D147_17 | SEQ ID NO: 247 |
| O75_9 | SEQ ID NO: 248 |
| AS152_1, AS152_2 | SEQ ID NO: 249 |
| AS167_3 | SEQ ID NO: 250 |
| AU122_1 | SEQ ID NO: 251 |
| BF208_1 | SEQ ID NO: 252 |
| BG513_19 | SEQ ID NO: 253 |
| BG556_8 | SEQ ID NO: 254 |
| C195_1, C195_4 | SEQ ID NO: 255 |
| O276_16 | SEQ ID NO: 256 |
| AC41_1 | SEQ ID NO: 257 |
| AC222_1 | SEQ ID NO: 258 |
| AJ143_1 | SEQ ID NO: 259 |
| AJ168_4 | SEQ ID NO: 260 |
| AK684_1 | SEQ ID NO: 261 |
| AS209_1 | SEQ ID NO: 262 |
| AX56_28 | SEQ ID NO: 263 |
| AX92_3 | SEQ ID NO: 264 |
| BF245_1 | SEQ ID NO: 265 |
| BG33_7 | SEQ ID NO: 266 |
| BM46_10 | SEQ ID NO: 267 |
| J317_1 | SEQ ID NO: 268 |
| O289_1 | SEQ ID NO: 269 |
| AJ26_3 | SEQ ID NO: 270 |
| AJ172_2 | SEQ ID NO: 271 |
| AP224_2 | SEQ ID NO: 272 |
| BL89_13 | SEQ ID NO: 273 |
| BL341_4 | SEQ ID NO: 274 |
| BV239_3 | SEQ ID NO: 275 |
| CC25_17 | SEQ ID NO: 276 |
| CC397_19 | SEQ ID NO: 277 |
| D305_2 | SEQ ID NO: 278 |
| G55_1 | SEQ ID NO: 279 |
| K39_7 | SEQ ID NO: 280 |
| K330_3 | SEQ ID NO: 281 |
| K363_3 | SEQ ID NO: 282 |
| K446_3 | SEQ ID NO: 283 |
| K464_4 | SEQ ID NO: 284 |
| K483_1 | SEQ ID NO: 285 |
| L69_3 | SEQ ID NO: 286 |
| BG511_30 | SEQ ID NO: 287 |
| BL15_12 | SEQ ID NO: 288 |
| K289_4 | SEQ ID NO: 289 |
| K322_4 | SEQ ID NO: 290 |
| AM349_2 | SEQ ID NO: 291 |
| AR310_3 | SEQ ID NO: 292 |
| AS186_3 | SEQ ID NO: 293 |
| AY160_2 | SEQ ID NO: 294 |
| BD127_16 | SEQ ID NO: 295 |
| BL205_14 | SEQ ID NO: 296 |
| H438_1 | SEQ ID NO: 297 |
| AY421_2 | SEQ ID NO: 298 |
| BV278_2 | SEQ ID NO: 299 |
| C544_1 | SEQ ID NO: 300 |
| CC332_33 | SEQ ID NO: 301 |
| CC365_40 | SEQ ID NO: 302 |
| CG68_4 | SEQ ID NO: 303 |
| D329_1 | SEQ ID NO: 304 |
| H698_3 | SEQ ID NO: 305 |
| H963_20 | SEQ ID NO: 306 |
| BD372_5 | SEQ ID NO: 307 |
| BR533_4 | SEQ ID NO: 308 |
| CC288_9 | SEQ ID NO: 309 |

In the sequences listed above which include an N at position 2, that position is occupied in preferred probes/primers by a biotinylated phosphoaramidite residue rather than a nucleotide (such as, for example, that produced by use of biotin phosphoramidite (1-dimethoxytrityloxy-2-(N-biotinyl-4-aminobutyl)-propyl-3-O-(2-cyanoethyl)-(N,N-diisopropyl)-phosphoramadite) (Glen Research, cat. no. 10-1953)).

The design of the oligonucleotide probe should preferably follow these parameters:
(a) It should be designed to an area of the sequence which has the fewest ambiguous bases (“N's”), if any;
(b) It should be designed to have a Tₘ of approx. 80° C. (assuming 2° for each A or T and 4 degrees for each G or C).
   The oligonucleotide should preferably be labeled with γ-³²P ATP (specific activity 6000 Ci/mmole) and T4 polynucleotide kinase using commonly employed techniques for labeling oligonucleotides. Other labeling techniques can also be used. Unincorporated label should preferably be removed by gel filtration chromatography or other established methods. The amount of radioactivity incorporated into the probe should be quantitated by measurement in a scintillation counter. Preferably, specific activity of the resulting probe should be approximately 4e+6 dpm/pmole.

The bacterial culture containing the pool of full-length clones should preferably be thawed and 100 μl of the stock used to inoculate a sterile culture flask containing 25 ml of sterile L-broth containing ampicillin at 100 μg/ml. The culture should preferably be grown to saturation at 37° C., and the saturated culture should preferably be diluted in fresh L-broth. Aliquots of these dilutions should preferably be plated to determine the dilution and volume which will yield approximately 5000 distinct and well-separated colonies on solid bacteriological media containing L-broth containing ampicillin at 100 μg/ml and agar at 1.5% in a 150 mm petri dish when grown overnight at 37° C. Other known methods of obtaining distinct, well-separated colonies can also be employed.

Standard colony hybridization procedures should then be used to transfer the colonies to nitrocellulose filters and lyse, denature and bake them.

The filter is then preferably incubated at 65° C. for 1 hour with gentle agitation in 6×SSC (20× stock is 175.3 g NaCl/liter, 88.2 g Na citrate/liter, adjusted to pH 7.0 with NaOH) containing 0.5% SDS, 100 μg/ml of yeast RNA, and 10 mM EDTA (approximately 10 mL per 150 mm filter). Preferably, the probe is then added to the hybridization mix at a concentration greater than or equal to 1e+6 dpm/mL. The filter is then preferably incubated at 65° C. with gentle agitation overnight. The filter is then preferably washed in 500 mL of 2×SSC/0.5% SDS at room temperature without agitation, preferably followed by 500 mL of 2×SSC/0.1% SDS at room temperature with gentle shaking for 15 minutes. A third wash with 0.1×SSC/0.5% SDS at 65° C. for 30 minutes to 1 hour is optional. The filter is then preferably dried and subjected to autoradiography for sufficient time to visualize the positives on the X-ray film. Other known hybridization methods can also be employed.

The positive colonies are picked, grown in culture, and plasmid DNA isolated using standard procedures. The clones can then be verified by restriction analysis, hybridization analysis, or DNA sequencing.

Fragments of the proteins of the present invention which are capable of exhibiting biological activity are also encompassed by the present invention. Fragments of the protein may be in linear form or they may be cyclized using known methods, for example, as described in H. U. Saragovi, et al., Bio/Technology 10, 773-778 (1992) and in R. S. McDowell, et al., J. Amer. Chem. Soc. 114, 9245-9253 (1992), both of which are incorporated herein by reference. Such fragments may be fused to carrier molecules such as immunoglobulins for many purposes, including increasing the valency of protein binding sites. For example, fragments of the protein may be fused through “linker” sequences to the Fc portion of an immunoglobulin. For a bivalent form of the protein, such a fusion could be to the Fc portion of an IgG molecule. Other immunoglobulin isotypes may also be used to generate such fusions. For example, a protein-IgM fusion would generate a decavalent form of the protein of the invention.

The present invention also provides both full-length and mature forms of the disclosed proteins. The full-length form of the such proteins is identified in the sequence listing by translation of the nucleotide sequence of each disclosed clone. The mature form(s) of such protein may be obtained by expression of the disclosed full-length polynucleotide (preferably those deposited with ATCC) in a suitable mammalian cell or other host cell. The sequence(s) of the mature form(s) of the protein may also be determinable from the amino acid sequence of the full-length form.

The present invention also provides genes corresponding to the polynucleotide sequences disclosed herein. “Corresponding genes” are the regions of the genome that are transcribed to produce the mRNAs from which cDNA polynucleotide sequences are derived and may include contiguous regions of the genome necessary for the regulated expression of such genes. Corresponding genes may therefore include but are not limited to coding sequences, 5′ and 3′ untranslated regions, alternatively spliced exons, introns, promoters, enhancers, and silencer or suppressor elements. The corresponding genes can be isolated in accordance with known methods using the sequence information disclosed herein. Such methods include the preparation of probes or primers from the disclosed sequence information for identification and/or amplification of genes in appropriate genomic libraries or other sources of genomic materials. An “isolated gene” is a gene that has been separated from the adjacent coding sequences, if any, present in the genome of the organism from which the gene was isolated.

The chromosomal location corresponding to the polynucleotide sequences disclosed herein may also be determined, for example by hybridizing appropriately labeled polynucleotides of the present invention to chromosomes in situ. It may also be possible to determine the corresponding chromosomal location for a disclosed polynucleotide by identifying significantly similar nucleotide sequences in public databases, such as expressed sequence tags (ESTs), that have already been mapped to particular chromosomal locations. For at least some of the polynucleotide sequences disclosed herein, public database sequences having at least some similarity to the polynucleotide of the present invention have been listed by database accession number. Searches using the GenBank accession numbers of these public database sequences can then be performed at an Internet site provided by the National Center for Biotechnology Information having the address http://www.ncbi.nlm.nih.gov/UniGene/, in order to identify “UniGene clusters” of overlapping sequences. Many of the “UniGene clusters” so identified will already have been mapped to particular chromosomal sites.

Organisms that have enhanced, reduced, or modified expression of the gene(s) corresponding to the polynucleotide sequences disclosed herein are provided. The desired change in gene expression can be achieved through the use of antisense polynucleotides or ribozymes that bind and/or cleave the mRNA transcribed from the gene (Albert and Morris, 1994, Trends Pharmacol. Sci. 15(7): 250-254; Lavarosky et al., 1997, Biochem. Mol. Med. 62(1): 11-22; and Hampel, 1998, Prog. Nucleic Acid Res. Mol. Biol. 58: 1-39; all of which are incorporated by reference herein). The desired change in gene expression can also be achieved through the use of double-stranded ribonucleotide molecules having some complementarity to the mRNA transcribed from the gene, and which interfere with the transcription, stability, or expression of the mRNA (“RNA intereference” or “RNAi”; Fire et al., 1998, Nature 391 (6669): 806-811; Montgomery et al., 1998, Proc. Natl. Acad. Sci. USA 95 (26): 15502-15507; and Sharp, 1999, Genes Dev. 13 (2): 139-141; all of which are incorporated by reference herein). Transgenic animals that have multiple copies of the gene(s) corresponding to the polynucleotide sequences disclosed herein, preferably produced by transformation of cells with genetic constructs that are stably maintained within the transformed cells and their progeny, are provided. Transgenic animals that have modified genetic control regions that increase or reduce gene expression levels, or that change temporal or spatial patterns of gene expression, are also provided (see European Patent No. 0 649 464 B1, incorporated by reference herein). In addition, organisms are provided in which the gene(s) corresponding to the polynucleotide sequences disclosed herein have been partially or completely inactivated, through insertion of extraneous sequences into the corresponding gene(s) or through deletion of all or part of the corresponding gene(s). Partial or complete gene inactivation can be accomplished through insertion, preferably followed by imprecise excision, of transposable elements (Plasterk, 1992, Bioessays 14(9): 629-633; Zwaal et al., 1993, Proc. Natl. Acad. Sci. USA 90(16): 7431-7435; Clark et al., 1994, Proc. Natl. Acad. Sci. USA 91(2): 719-722; all of which are incorporated by reference herein), or through homologous recombination, preferably detected by positive/negative genetic selection strategies (Mansour et al., 1988, Nature 336: 348-352; U.S. Pat. Nos. 5,464,764; 5,487,992; 5,627,059; 5,631,153; 5,614,396; 5,616,491; and 5,679,523; all of which are incorporated by reference herein). These organisms with altered gene expression are preferably eukaryotes and more preferably are mammals. Such organisms are useful for the development of non-human models for the study of disorders involving the corresponding gene(s), and for the development of assay systems for the identification of molecules that interact with the protein product(s) of the corresponding gene(s).

Where the protein of the present invention is membrane-bound (e.g., is a receptor), the present invention also provides for soluble forms of such protein. In such forms, part or all of the intracellular and transmembrane domains of the protein are deleted such that the protein is fully secreted from the cell in which it is expressed. The intracellular and transmembrane domains of proteins of the invention can be identified in accordance with known techniques for determination of such domains from sequence information. For example, the TopPredII computer program can be used to predict the location of transmembrane domains in an amino acid sequence, domains which are described by the location of the center of the transmsmbrane domain, with at least ten transmembrane amino acids on each side of the reported central residue(s).

Proteins and protein fragments of the present invention include proteins with amino acid sequence lengths that are at least 25% (more preferably at least 50%, and most preferably at least 75%) of the length of a disclosed protein and have at least 60% sequence identity (more preferably, at least 75% identity; most preferably at least 90% or 95% identity) with that disclosed protein, where sequence identity is determined by comparing the amino acid sequences of the proteins when aligned so as to maximize overlap and identity while minimizing sequence gaps. Also included in the present invention are proteins and protein fragments that contain a segment preferably comprising 8 or more (more preferably 20 or more, most preferably 30 or more) contiguous ammo acids that shares at least 75% sequence identity (more preferably, at least 85% identity; most preferably at least 95% identity) with any such segment of any of the disclosed proteins.

In particular, sequence identity may be determined using WU-BLAST (Washington University BLAST) version 2.0 software, which builds upon WU-BLAST version 1.4, which in turn is based on the public domain NCBI-BLAST version 1.4 (Altschul and Gish, 1996, Local alignment statistics, Doolittle ed., Methods in Enzymology 266: 460-480; Altschul et al., 1990, Basic local alignment search tool, Journal of Molecular Biology 215: 403-410; Gish and States, 1993, Identification of protein coding regions by database similarity search, Nature Genetics 3: 266-272; Karlin and Altschul, 1993, Applications and statistics for multiple high-scoring segments in molecular sequences, Proc. Natl. Acad. Sci. USA 90: 5873-5877; all of which are incorporated by reference herein). WU-BLAST version 2.0 executable programs for several UNIX platforms can be downloaded from ftp://blast.wustl.edu/blast/executables. The complete suite of search programs (BLASTP, BLASTN, BLASTX, TBLASTN, and TBLASTX) is provided at that site, in addition to several support programs. WU-BLAST 2.0 is copyrighted and may not be sold or redistributed in any form or manner without the express written consent of the author; but the posted executables may otherwise be freely used for commercial, nonprofit, or academic purposes. In all search programs in the suite—BLASTP, BLASTN, BLASTX, TBLASTN and TBLASTX—the gapped alignment routines are integral to the database search itself, and thus yield much better sensitivity and selectivity while producing the more easily interpreted output. Gapping can optionally be turned off in all of these programs, if desired. The default penalty (Q) for a gap of length one is Q=9 for proteins and BLASTP, and Q=10 for BLASTN, but may be changed to any integer value including zero, one through eight, nine, ten, eleven, twelve through twenty, twenty-one through fifty, fifty-one through one hundred, etc. The default per-residue penalty for extending a gap (R) is R=2 for proteins and BLASTP, and R=10 for BLASTN, but may be changed to any integer value including zero, one, two, three, four, five, six, seven, eight, nine, ten, eleven, twelve through twenty, twenty-one through fifty, fifty-one through one hundred, etc. Any combination of values for Q and R can be used in order to align sequences so as to maximize overlap and identity while minimizing sequence gaps. The default amino acid comparison matrix is BLOSUM62, but other amino acid comparison matrices such as PAM can be utilized.

Species homologues of the disclosed polynucleotides and proteins are also provided by the present invention. As used herein, a “species homologue” is a protein or polynucleotide with a different species of origin from that of a given protein or polynucleotide, but with significant sequence similarity to the given protein or polynucleotide. Preferably, polynucleotide species homologues have at least 60% sequence identity (more preferably, at least 75% identity; most preferably at least 90% identity) with the given polynucleotide, and protein species homologues have at least 30% sequence identity (more preferably, at least 45% identity; most preferably at least 60% identity) with the given protein, where sequence identity is determined by comparing the nucleotide sequences of the polynucleotides or the amino acid sequences of the proteins when aligned so as to maximize overlap and identity while minimizing sequence gaps. Species homologues may be isolated and identified by making suitable probes or primers from the sequences provided herein and screening a suitable nucleic acid source from the desired species. Preferably, species homologues are those isolated from mammalian species. Most preferably, species homologues are those isolated from certain mammalian species such as, for example, Pan troglodytes, Gorilla gorilla, Pongo pygmaeus, Hylobates concolor, Macaca mulatta, Papio papio, Papio hamadryas, Cercopithecus aethiops, Cebus capucinus, Aotus trivirgatus, Sanguinus oediptus, Microcebus murinus, Mus musculus, Rattus norvegicus, Cricetulus griseus, Felis catus, Mustela vison, Canis familiaris, Oryctolagus cuniculus, Bos taurus, Ovis aries, Sus scrofa, and Equus caballis, for which genetic maps have been created allowing the identification of syntenic relationships between the genomic organization of genes in one species and the genomic organization of the related genes in another species (O'Brien and Seuánez, 1988, Ann. Rev. Genet. 22: 323-351; O'Brien et al., 1993, Nature Genetics 3:103-112; Johansson et al., 1995, Genomics 25: 682-690; Lyons et al., 1997, Nature Genetics 15: 47-56; O'Brien et al., 1997, Trends in Genetics 13(10): 393-399; Carver and Stubbs, 1997, Genome Research 7:1123-1137; all of which are incorporated by reference herein).

The invention also encompasses allelic variants of the disclosed polynucleotides or proteins; that is, naturally-occurring alternative forms of the isolated polynucleotides which also encode proteins which are identical or have significantly similar sequences to those encoded by the disclosed polynucleotides. Preferably, allelic variants have at least 60% sequence identity (more preferably, at least 75% identity; most preferably at least 90% identity) with the given polynucleotide, where sequence identity is determined by comparing the nucleotide sequences of the polynucleotides when aligned so as to maximize overlap and identity while minimizing sequence gaps. Allelic variants may be isolated and identified by making suitable probes or primers from the sequences provided herein and screening a suitable nucleic acid source from individuals of the appropriate species.

The invention also includes polynucleotides with sequences complementary to those of the polynucleotides disclosed herein.

The present invention also includes polynucleotides that hybridize under reduced stringency conditions, more preferably stringent conditions, and most preferably highly stringent conditions, to polynucleotides described herein. Examples of stringency conditions are shown in the table below: highly stringent conditions are those that are at least as stringent as, for example, conditions A-F; stringent conditions are at least as stringent as, for example, conditions G-L; and reduced stringency conditions are at least as stringent as, for example, conditions M-R.

| | | | | |
|---|---|---|---|---|
| | Poly- | Hybrid | Temperature and | Wash |
| Stringency | nucleotide | Length | Hybridization | Temperature |
| Condition | Hybrid | (bp)^{‡} | Buffer† | and Buffer† |

| A | DNA:DNA | ≧50 | 65° C.; 1×SSC -or- | 65° C.; |
|---|---|---|---|---|
| | | | 42° C.; 1×SSC, | 0.3×SSC |
| | | | 50% formamide | |
| B | DNA:DNA | <50 | T_{B}*; 1×SSC | T_{B}*; |
| | | | | 1×SSC |
| C | DNA:RNA | ≧50 | 67° C.; 1×SSC -or- | 67° C.; |
| | | | 45° C.; 1×SSC, | 0.3×SSC |
| | | | 50% formamide | |
| D | DNA:RNA | <50 | T_{D}*; 1×SSC | T_{D}*; |
| | | | | 1×SSC |
| E | RNA:RNA | ≧50 | 70° C.; 1×SSC -or- | 70° C.; |
| | | | 50° C.; 1×SSC, | 0.3×SSC |
| | | | 50% formamide | |
| F | RNA:RNA | <50 | T_{F}*; 1×SSC | T_{F}*; |
| | | | | 1×SSC |
| G | DNA:DNA | ≧50 | 65° C.; 4×SSC -or- | 65° C.; |
| | | | 42° C.; 4×SSC, | 1×SSC |
| | | | 50% formamide | |
| H | DNA:DNA | <50 | T_{H}*; 4×SSC | T_{H}*; |
| | | | | 4×SSC |
| I | DNA:RNA | ≧50 | 67° C.; 4×SSC -or- | 67° C.; |
| | | | 45° C.; 4×SSC, | 1×SSC |
| | | | 50% formamide | |
| J | DNA:RNA | <50 | T_{J}*; 4×SSC | T_{J}*; |
| | | | | 4×SSC |
| K | RNA:RNA | ≧50 | 70° C.; 4×SSC -or- | 67° C.; |
| | | | 50° C.; 4×SSC, | 1×SSC |
| | | | 50% formamide | |
| L | RNA:RNA | <50 | T_{L}*; 2×SSC | T_{L}*; |
| | | | | 2×SSC |
| M | DNA:DNA | ≧50 | 50° C.; 4×SSC -or- | 50° C.; |
| | | | 40° C.; 6×SSC, | 2×SSC |
| | | | 50% formamide | |
| N | DNA:DNA | <50 | T_{N}; 6×SSC | T_{N}*; |
| | | | | 6×SSC |
| O | DNA:RNA | ≧50 | 55° C.; 4×SSC -or- | 55° C.; |
| | | | 42° C.; 6×SSC, | 2×SSC |
| | | | 50% formamide | |
| P | DNA:RNA | <50 | T_{P}*; 6×SSC | T_{P}*; |
| | | | | 6×SSC |
| Q | RNA:RNA | ≧50 | 60° C.; 4×SSC -or- | 60° C.; |
| | | | 45° C.; 6×SSC, | 2×SSC |
| | | | 50% formamide | |
| R | RNA:RNA | <50 | T_{R}*; 4×SSC | T_{R}*; |
| | | | | 4×SSC |
| ^{‡}: The hybrid length is that anticipated for the hybridized region(s) of the hybridizing polynucleotides. When hybridizing a polynucleotide to a target polynucleotide of unknown sequence, the hybrid length is assumed to be that of the hybridizing polynudeotide. When polynucleoL-ides of known sequence are hybridized, the hybrid length can be deternuned by aligning the sequences of the polynucleotides and identifying the region or regions of optimal sequence complementarity. | | | | |
| ^{†}: SSPE (1×SSPE is 0.15 M NaCl, 10 mM NaH₂PO_{4/} and 1.25 mM EDTA, pH 7.4) can be substituted for SSC (1×SSC is 0.15 M NaCl and 15 mM sodium citrate) in the hybridization and wash buffers; washes are performed for 15 minutes after hybridization is complete. | | | | |
| *T_{B}-T_{R}: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For hybrids between 18 and 49 base paris in length, Tₘ(° C.) = | | | | |
| # 81.5 + 16.6(log₁₀[Na^{+]) + 0.41(% G+C)-(600/N), where N is the number of bases in the hybrid, and [Na}⁺] is the concentration of sodium ions in the hybridization buffer ([Na⁺] for 1×SSC = 0.165 M). | | | | |

Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

Preferably, each such hybridizing polynucleotide has a length that is at least 25% (more preferably at least 50%, and most preferably at least 75%) of the length of the polynucleotide of the present invention to which it hybridizes, and has at least 60% sequence identity (more preferably, at least 75% identity; most preferably at least 90% or 95% identity) with the polynucleotide of the present invention to which it hybridizes, where sequence identity is determined by comparing the sequences of the hybridizing polynucleotides when aligned so as to maximize overlap and identity while minimizing sequence gaps.

The isolated polynucleotide endcoing the protein of the invention may be operably linked to an expression control sequence such as the pMT2 or pED expression vectors disclosed in Kaufman et al., Nucleic Acids Res. 19, 4485-4490 (1991), in order to produce the protein recombinantly. Many suitable expression control sequences are known in the art. General methods of expressing recombinant proteins are also known and are exemplified in R. Kaufman, Methods in Enzymology 185, 537-566 (1990). As defined herein “operably linked” means that the isolated polynucleotide of the invention and an expression control sequence are situated within a vector or cell in such a way that the protein is expressed by a host cell which has been transformed (transfected) with the ligated polynucleotide/expression control sequence.

A number of types of cells may act as suitable host cells for expression of the protein. Mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells.

Alternatively, it may be possible to produce the protein in lower eukaryotes such as yeast or in prokaryotes such as bacteria. Potentially suitable yeast strains include Saccharomyces cerevisiae, Schizosaccharomyces pombe, Kluyveromyces strains, Candida, or any yeast strain capable of expressing heterologous proteins. Potentially suitable bacterial strains include Escherichia coli, Bacillus subtilis, Salmonella typhimurium, or any bacterial strain capable of expressing heterologous proteins. If the protein is made in yeast or bacteria, it may be necessary to modify the protein produced therein, for example by phosphorylation or glycosylation of the appropriate sites, in order to obtain the functional protein. Such covalent attachments may be accomplished using known chemical or enzymatic methods.

The protein may also be produced by operably linking the isolated polynucleotide of the invention to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBac® kit), and such methods are well known in the art, as described in Summers and Smith, Texas Agricultural Experiment Station Bulletin No. 1555 (1987), incorporated herein by reference. As used herein, an insect cell capable of expressing a polynucleotide of the present invention is “transformed.”

The protein of the invention may be prepared by culturing transformed host cells under culture conditions suitable to express the recombinant protein. The resulting expressed protein may then be purified from such culture (i.e., from culture medium or cell extracts) using known purification processes, such as gel filtration and ion exchange chromatography. The purification of the protein may also include an affinity column containing agents which will bind to the protein; one or more column steps over such affinity resins as concanavalin A-agarose, heparin-toyopearl® or Cibacrom blue 3GA Sepharose®; one or more steps involving hydrophobic interaction chromatography using such resins as phenyl ether, butyl ether, or propyl ether; or immunoaffinity chromatography.

Alternatively, the protein of the invention may also be expressed in a form which will facilitate purification. For example, it may be expressed as a fusion protein, such as those of maltose binding protein (MBP), glutathione-S-transferase (GST) or thioredoxin (TRX). Kits for expression and purification of such fusion proteins are commercially available from New England BioLabs (Beverly, Mass.), Pharmacia (Piscataway, N.J.) and Invitrogen Corporation (Carlsbad, Calif.), respectively. The protein can also be tagged with an epitope and subsequently purified by using a specific antibody directed to such epitope. One such epitope (“Flag”) is commercially available from the Eastman Kodak Company (New Haven, Conn.).

Finally, one or more reverse-phase high performance liquid chromatography (RP-HPLC) steps employing hydrophobic RP-HPLC media, e.g., silica gel having pendant methyl or other aliphatic groups, can be employed to further purify the protein. Some or all of the foregoing purification steps, in various combinations, can also be employed to provide a substantially homogeneous isolated recombinant protein. The protein thus purified is substantially free of other mammalian proteins and is defined in accordance with the present invention as an “isolated protein.”

The protein of the invention may also be expressed as a product of transgenic animals, e.g., as a component of the milk of transgenic cows, goats, pigs, or sheep which are characterized by somatic or germ cells containing a nucleotide sequence encoding the protein.

The protein may also be produced by known conventional chemical synthesis. Methods for constructing the proteins of the present invention by synthetic means are known to those skilled in the art. The synthetically-constructed protein sequences, by virtue of sharing primary, secondary or tertiary structural and/or conformational characteristics with proteins may possess biological properties in common therewith, including protein activity. Thus, they may be employed as biologically active or immunological substitutes for natural, purified proteins in screening of therapeutic compounds and in immunological processes for the development of antibodies.

The proteins provided herein also include proteins characterized by amino acid sequences similar to those of purified proteins but into which modification are naturally provided or deliberately engineered. For example, modifications in the peptide or DNA sequences can be made by those skilled in the art using known techniques. Modifications of interest in the protein sequences may include the alteration, substitution, replacement, insertion or deletion of a selected amino acid residue in the coding sequence. For example, one or more of the cysteine residues may be deleted or replaced with another amino acid to alter the conformation of the molecule. Techniques for such alteration, substitution, replacement, insertion or deletion are well known to those skilled in the art (see, e.g., U.S. Pat. No. 4,518,584). Preferably, such alteration, substitution, replacement, insertion or deletion retains the desired activity of the protein.

Other fragments and derivatives of the sequences of proteins which would be expected to retain protein activity in whole or in part and may thus be useful for screening or other immunological methodologies may also be easily made by those skilled in the art given the disclosures herein. Such modifications are believed to be encompassed by the present invention.

Uses and Biological Activity

The polynucleotides and proteins of the present invention are expected to exhibit one or more of the uses or biological activities (including those associated with assays cited herein) identified below. Uses or activities described for proteins of the present invention may be provided by administration or use of such proteins or by administration or use of polynucleotides encoding such proteins (such as, for example, in gene therapies or vectors suitable for introduction of DNA).

Research Uses and Utilities

The polynucleotides provided by the present invention can be used by the research community for various purposes. The polynucleotides can be used to express recombinant protein for analysis, characterization or therapeutic use; as markers for tissues in which the corresponding protein is preferentially expressed (either constitutively or at a particular stage of tissue differentiation or development or in disease states); as molecular weight markers on Southern gels; as chromosome markers or tags (when labeled) to identify chromosomes or to map related gene positions; to compare with endogenous DNA sequences in patients to identify potential genetic disorders; as probes to hybridize and thus discover novel, related DNA sequences; as a source of information to derive PCR primers for genetic fingerprinting; as a probe to “subtract-out” known sequences in the process of discovering other novel polynucleotides; for selecting and making oligomers for attachment to a “gene chip” or other support, including for examination of expression patterns; to raise anti-protein antibodies using DNA immunization techniques; and as an antigen to raise anti-DNA antibodies or elicit another immune response. Where the polynucleotide encodes a protein which binds or potentially binds to another protein (such as, for example, in a receptor-ligand interaction), the polynucleotide can also be used in interaction trap assays (such as, for example, those described in Gyuris et al., 1993, Cell 75: 791-803 and in Rossi et al., 1997, Proc. Natl. Acad. Sci. USA 94: 8405-8410, all of which are incorporated by reference herein) to identify polynucleotides encoding the other protein with which binding occurs or to identify inhibitors of the binding interaction.

The proteins provided by the present invention can similarly be used in assay to determine biological activity, including in a panel of multiple proteins for high-throughput screening; to raise antibodies or to elicit another immune response; as a reagent (including the labeled reagent) in assays designed to quantitatively determine levels of the protein (or its receptor) in biological fluids; as markers for tissues in which the corresponding protein is preferentially expressed (either constitutively or at a particular stage of tissue differentiation or development or in a disease state); and, of course, to isolate correlative receptors or ligands. Where the protein binds or potentially binds to another protein (such as, for example, in a receptor-ligand interaction), the protein can be used to identify the other protein with which binding occurs or to identify inhibitors of the binding interaction. Proteins involved in these binding interactions can also be used to screen for peptide or small molecule inhibitors or agonists of the binding interaction.

Any or all of these research utilities are capable of being developed into reagent grade or kit format for commercialization as research products.

Methods for performing the uses listed above are well known to those skilled in the art. References disclosing such methods include without limitation “Molecular Cloning: A Laboratory Manual”, 2d ed., Cold Spring Harbor Laboratory Press, Sambrook, J., E. F. Fritsch and T. Maniatis eds., 1989, and “Methods in Enzymology: Guide to Molecular Cloning Techniques”, Academic Press, Berger, S. L. and A. R. Kimmel eds., 1987.

Nutritional Uses

Polynucleotides and proteins of the present invention can also be used as nutritional sources or supplements. Such uses include without limitation use as a protein or amino acid supplement, use as a carbon source, use as a nitrogen source and use as a source of carbohydrate. In such cases the protein or polynucleotide of the invention can be added to the feed of a particular organism or can be administered as a separate solid or liquid preparation, such as in the form of powder, pills, solutions, suspensions or capsules. In the case of microorganisms, the protein or polynucleotide of the invention can be added to the medium in or on which the microorganism is cultured.

Cytokine and Cell Proliferation/Differentiation Activity

A protein of the present invention may exhibit cytokine, cell proliferation (either inducing or inhibiting) or cell differentiation (either inducing or inhibiting) activity or may induce production of other cytokines in certain cell populations. Many protein factors discovered to date, including all known cytokines, have exhibited activity in one or more factor-dependent cell proliferation assays, and hence the assays serve as a convenient confirmation of cytokine activity. The activity of a protein of the present invention is evidenced by any one of a number of routine factor dependent cell proliferation assays for cell lines including, without limitation, 32D, DA2, DA1G, T10, B9, B9/11, BaF3, MC9/G, M+ (preB M+), 2E8, RB5, DA1, 123, T1165, HT2, CTLL2, TF-1, Mo7e and CMK.

The activity of a protein of the invention may, among other means, be measured by the following methods:

Assays for T-cell or thymocyte proliferation include without limitation those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Takai et al., J. Immunol. 137:3494-3500, 1986; Bertagnolli et al., J. Immunol. 145:1706-1712, 1990; Bertagnolli et al., Cellular Immunology 133:327-341, 1991; Bertagnolli, et al., J. Immunol. 149:3778-3783, 1992; Bowman et al., J. Immunol. 152: 1756-1761, 1994.

Assays for cytokine production and/or proliferation of spleen cells, lymph node cells or thymocytes include, without limitation, those described in: Polyclonal T cell stimulation, Kruisbeek, A. M. and Shevach, E. M. In Current Protocols in Immunology. J.E.e.a. Coligan eds. Vol 1 pp. 3.12.1-3.12.14, John Wiley and Sons, Toronto. 1994; and Measurement of mouse and human Interferon γ, Schreiber, R. D. In Current Protocols in Immunology. J.E.e.a. Coligan eds. Vol 1 pp. 6.8.1-6.8.8, John Wiley and Sons, Toronto. 1994.

Assays for proliferation and differentiation of hematopoietic and lymphopoietic cells include, without limitation, those described in: Measurement of Human and Murine Interleukin 2 and Interleukin 4, Bottomly, K., Davis, L. S. and Lipsky, P. E. In Current Protocols in Immunology. J.E.e.a. Coligan eds. Vol 1 pp. 6.3.1-6.3.12, John Wiley and Sons, Toronto. 1991; deVries et al., J. Exp. Med. 173:1205-1211, 1991; Moreau et al., Nature 336:690-692, 1988; Greenberger et al., Proc. Natl. Acad. Sci. U.S.A. 80:2931-2938, 1983; Measurement of mouse and human interleukin 6—Nordan, R. In Current Protocols in Immunology. J.E.e.a. Coligan eds. Vol 1 pp. 6.6.1-6.6.5, John Wiley and Sons, Toronto. 1991; Smith et al., Proc. Natl. Acad. Sci. U.S.A. 83:1857-1861, 1986; Measurement of human Interleukin 11—Bennett, F., Giannotti, J., Clark, S. C. and Turner, K. J. In Current Protocols in Immunology. J.E.e.a. Coligan eds. Vol 1 pp. 6.15.1 John Wiley and Sons, Toronto. 1991;

Measurement of mouse and human Interleukin 9—Ciarletta, A., Giannotti, J., Clark, S. C. and Turner, K. J. In Current Protocols in Immunology. J.E.e.a. Coligan eds. Vol 1 pp. 6.13.1, John Wiley and Sons, Toronto. 1991.

Assays for T-cell clone responses to antigens (which will identify, among others, proteins that affect APC-T cell interactions as well as direct T-cell effects by measuring proliferation and cytokine production) include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function; Chapter 6, Cytokines and their cellular receptors; Chapter 7, Immunologic studies in Humans); Weinberger et al., Proc. Natl. Acad. Sci. USA 77:6091-6095, 1980; Weinberger et al., Eur. J. Immun. 11:405-411, 1981; Takai et al., J. Immunol. 137:3494-3500, 1986; Takai et al., J. Immunol. 140:508-512, 1988.

Immune Stimulating or Suppressing Activity

A protein of the present invention may also exhibit immune stimulating or immune suppressing activity, including without limitation the activities for which assays are described herein. A protein may be useful in the treatment of various immune deficiencies and disorders (including severe combined immunodeficiency (SCID)), e.g., in regulating (up or down) growth and proliferation of T and/or B lymphocytes, as well as effecting the cytolytic activity of NK cells and other cell populations. These immune deficiencies may be genetic or be caused by viral (e.g., HIV) as well as bacterial or fungal infections, or may result from autoimmune disorders. More specifically, infectious diseases causes by viral, bacterial, fungal or other infection may be treatable using a protein of the present invention, including infections by HIV, hepatitis viruses, herpesviruses, mycobacteria, Leishmania spp., malaria spp. and various fungal infections such as candidiasis. Of course, in this regard, a protein of the present invention may also be useful where a boost to the immune system generally may be desirable, i.e., in the treatment of cancer.

Autoimmune disorders which may be treated using a protein of the present invention include, for example, connective tissue disease, multiple sclerosis, systemic lupus erythematosus, rheumatoid arthritis, autoimmune pulmonary inflammation, Guillain-Barre syndrome, autoimmune thyroiditis, insulin dependent diabetes mellitis, myasthenia gravis, graft-versus-host disease and autoimmune inflammatory eye disease. Such a protein of the present invention may also to be useful in the treatment of allergic reactions and conditions, such as asthma (particularly allergic asthma) or other respiratory problems. Other conditions, in which immune suppression is desired (including, for example, organ transplantation), may also be treatable using a protein of the present invention.

Using the proteins of the invention it may also be possible to regulate immune responses in a number of ways. Down regulation may be in the form of inhibiting or blocking an immune response already in progress or may involve preventing the induction of an immune response. The functions of activated T cells may be inhibited by suppressing T cell responses or by inducing specific tolerance in T cells, or both. Immunosuppression of T cell responses is generally an active, non-antigen-specific, process which requires continuous exposure of the T cells to the suppressive agent. Tolerance, which involves inducing non-responsiveness or anergy in T cells, is distinguishable from immunosuppression in that it is generally antigen-specific and persists after exposure to the tolerizing agent has ceased. Operationally, tolerance can be demonstrated by the lack of a T cell response upon reexposure to specific antigen in the absence of the tolerizing agent.

Down regulating or preventing one or more antigen functions (including without limitation B lymphocyte antigen functions (such as, for example, B7)), e.g., preventing high level lymphokine synthesis by activated T cells, will be useful in situations of tissue, skin and organ transplantation and in graft-versus-host disease (GVHD). For example, blockage of T cell function should result in reduced tissue destruction in tissue transplantation. Typically, in tissue transplants, rejection of the transplant is initiated through its recognition as foreign by T cells, followed by an immune reaction that destroys the transplant. The administration of a molecule which inhibits or blocks interaction of a B7 lymphocyte antigen with its natural ligand(s) on immune cells (such as a soluble, monomeric form of a peptide having B7-2 activity alone or in conjunction with a monomeric form of a peptide having an activity of another B lymphocyte antigen (e.g., B7-1, B7-3) or blocking antibody), prior to transplantation can lead to the binding of the molecule to the natural ligand(s) on the immune cells without transmitting the corresponding costimulatory signal. Blocking B lymphocyte antigen function in this matter prevents cytokine synthesis by immune cells, such as T cells, and thus acts as an immunosuppressant. Moreover, the lack of costimulation may also be sufficient to anergize the T cells, thereby inducing tolerance in a subject. Induction of long-term tolerance by B lymphocyte antigen-blocking reagents may avoid the necessity of repeated administration of these blocking reagents. To achieve sufficient immunosuppression or tolerance in a subject, it may also be necessary to block the function of a combination of B lymphocyte antigens.

The efficacy of particular blocking reagents in preventing organ transplant rejection or GVHD can be assessed using animal models that are predictive of efficacy in humans. Examples of appropriate systems which can be used include allogeneic cardiac grafts in rats and xenogeneic pancreatic islet cell grafts in mice, both of which have been used to examine the immunosuppressive effects of CTLA4 μg fusion proteins in vivo as described in Lenschow et al., Science 257:789-792 (1992) and Turka et al., Proc. Natl. Acad. Sci USA, 89:11102-11105 (1992). In addition, murine models of GVHD (see Paul ed., Fundamental Immunology, Raven Press, New York, 1989, pp. 846-847) can be used to determine the effect of blocking B lymphocyte antigen function in vivo on the development of that disease.

Blocking antigen function may also be therapeutically useful for treating autoimmune diseases. Many autoimmune disorders are the result of inappropriate activation of T cells that are reactive against self tissue and which promote the production of cytokines and autoantibodies involved in the pathology of the diseases. Preventing the activation of autoreactive T cells may reduce or eliminate disease symptoms. Administration of reagents which block costimulation of T cells by disrupting receptor:ligand interactions of B lymphocyte antigens can be used to inhibit T cell activation and prevent production of autoantibodies or T cell-derived cytokines which may be involved in the disease process. Additionally, blocking reagents may induce antigen-specific tolerance of autoreactive T cells which could lead to long-term relief from the disease. The efficacy of blocking reagents in preventing or alleviating autoimmune disorders can be determined using a number of well-characterized animal models of human autoimmune diseases. Examples include murine experimental autoimmune encephalitis, systemic lupus erythmatosis in MRL/lpr/lpr mice or NZB hybrid mice, murine autoimmune collagen arthritis, diabetes mellitus in NOD mice and BB rats, and murine experimental myasthenia gravis (see Paul ed., Fundamental Immunology, Raven Press, New York, 1989, pp. 840-856).

Upregulation of an antigen function (preferably a B lymphocyte antigen function), as a means of up regulating immune responses, may also be useful in therapy. Upregulation of immune responses may be in the form of enhancing an existing immune response or eliciting an initial immune response For example, enhancing an immune response through stimulating B lymphocyte antigen function may be useful in cases of viral infection. In addition, systemic viral diseases such as influenza, the common cold, and encephalitis might be alleviated by the administration of stimulatory forms of B lymphocyte antigens systemically.

Alternatively, anti-viral immune responses may be enhanced in an infected patient by removing T cells from the patient, costimulating the T cells in vitro with viral antigen-pulsed APCs either expressing a peptide of the present invention or together with a stimulatory form of a soluble peptide of the present invention and reintroducing the in vitro activated T cells into the patient. Another method of enhancing anti-viral immune responses would be to isolate infected cells from a patient, transfect them with a nucleic acid encoding a protein of the present invention as described herein such that the cells express all or a portion of the protein on their surface, and reintroduce the transfected cells into the patient. The infected cells would now be capable of delivering a costimulatory signal to, and thereby activate, T cells in vivo.

In another application, up regulation or enhancement of antigen function (preferably B lymphocyte antigen function) may be useful in the induction of tumor immunity. Tumor cells (e.g., sarcoma, melanoma, lymphoma, leukemia, neuroblastoma, carcinoma) transfected with a nucleic acid encoding at least one peptide of the present invention can be administered to a subject to overcome tumor-specific tolerance in the subject. If desired, the tumor cell can be transfected to express a combination of peptides. For example, tumor cells obtained from a patient can be transfected ex viva with an expression vector directing the expression of a peptide having B7-2-like activity alone, or in conjunction with a peptide having B7-1-like activity and/or B7-3-like activity. The transfected tumor cells are returned to the patient to result in expression of the peptides on the surface of the transfected cell. Alternatively, gene therapy techniques can be used to target a tumor cell for transfection in vivo.

The presence of the peptide of the present invention having the activity of a B lymphocyte antigen(s) on the surface of the tumor cell provides the necessary costimulation signal to T cells to induce a T cell mediated immune response against the transfected tumor cells. In addition, tumor cells which lack MHC class I or MHC class II molecules, or which fail to reexpress sufficient amounts of MHC class I or MHC class II molecules, can be transfected with nucleic acid encoding all or a portion of (e.g., a cytoplasmic-domain truncated portion) of an MHC class I α chain protein and β₂ microglobulin protein or an MHC class II α chain protein and an MHC class II β chain protein to thereby express MHC class I or MHC class II proteins on the cell surface. Expression of the appropriate class I or class II MHC in conjunction with a peptide having the activity of a B lymphocyte antigen (e.g., B7-1, B7-2, B7-3) induces a T cell mediated immune response against the transfected tumor cell. Optionally, a gene encoding an antisense construct which blocks expression of an MHC class II associated protein, such as the invariant chain, can also be cotransfected with a DNA encoding a peptide having the activity of a B lymphocyte antigen to promote presentation of tumor associated antigens and induce tumor specific immunity. Thus, the induction of a T cell mediated immune response in a human subject may be sufficient to overcome tumor-specific tolerance in the subject.

The activity of a protein of the invention may, among other means, be measured by the following methods:

Suitable assays for thymocyte or splenocyte cytotoxicity include, without limitation, those described in: Current Protocols in Immunology, Ed by J. E. Coligan, A. M. Kruisbeek, D. H. Margulies, E. M. Shevach, W Strober, Pub. Greene Publishing Associates and Wiley-Interscience (Chapter 3, In Vitro assays for Mouse Lymphocyte Function 3.1-3.19; Chapter 7, Immunologic studies in Humans); Herrmann et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Takai et al., J. Immunol. 140:508-512, 1988; Herrmann et al., Proc. Natl. Acad. Sci. USA 78:2488-2492, 1981; Herrmann et al., J. Immunol. 128:1968-1974, 1982; Handa et al., J. Immunol. 135:1564-1572, 1985; Takai et al., J. Immunol. 137:3494-3500, 1986; Bowman et al., J. Virology 61:1992-1998; Takai et al., J. Immunol. 140:508-512, 1988; Bertagnolli et al., Cellular Immunology 133:327-341, 1991; Brown et al., J. Immunol. 153:3079-3092, 1994.

Assays for T-cell-dependent immunoglobulin responses and isotype switching (which will identify, among others, proteins that modulate T-cell dependent antibody responses and that affect Th1/Th2 profiles) include, without limitation, those described in: Maliszewski, J. Immunol. 144:3028-3033, 1990; and Assays for B cell function: In vitro antibody production, Mond, J. J. and Brunswick, M. In Current Protocols in Immunology. J.E.e.a. Coligan eds. Vol 1 pp. 3.8.1-3.8.16, John Wiley and Sons, Toronto. 1994.

Mixed lymphocyte reaction (MLR) assays (which will identify, among others, proteins that generate predominantly Th1 and CTL responses) include, without limitation, those described in: Current Protocols in Immunology, Ed by J. E. Coligan, A. M. Kruisbeek, D. H. Margulies, E. M. Shevach, UV Strober, Pub. Greene Publishing Associates and Wiley-Interscience (Chapter 3, In Vitro assays for Mouse Lymphocyte Function 3.1-3.19; Chapter 7. Immunologic studies in Humans); Takai et al., J. Immunol. 137:3494-3500, 1986; Takai et al., J. Immunol. 140:508-512, 1988; Bertagnolli et al., J. Immunol. 149:3778-3783, 1992.

Dendritic cell-dependent assays (which will identify, among others, proteins expressed by dendritic cells that activate naive T-cells) include, without limitation, those described in: Guery et al., J. Immunol. 134:536-544, 1995; Inaba et al., Journal of Experimental Medicine 173:549-559, 1991; Macatonia et al., Journal of Immunology 154:5071-5079, 1995; Porgador et al., Journal of Experimental Medicine 182:255-260, 1995; Nair et al., Journal of Virology 67:4062-4069, 1993; Huang et al., Science 264:961-965, 1994; Macatonia et al., Journal of Experimental Medicine 169:1255-1264, 1989; Bhardwaj et al., Journal of Clinical Investigation 94:797-807, 1994; and Inaba et al., Journal of Experimental Medicine 172:631-640, 1990.

Assays for lymphocyte survival/apoptosis (which will identify, among others, proteins that prevent apoptosis after superantigen induction and proteins that regulate lymphocyte homeostasis) include, without limitation, those described in: Darzynkiewicz et al., Cytometry 13:795-808, 1992; Gorczyca et al., Leukemia 7:659-670, 1993; Gorczyca et al., Cancer Research 53:1945-1951, 1993; Itoh et al., Cell 66:233-243, 1991; Zacharchuk, Journal of Immunology 145:4037-4045, 1990; Zamai et al., Cytometry 14:891-897, 1993; Gorczyca et al., International Journal of Oncology 1:639-648, 1992.

Assays for proteins that influence early steps of T-cell commitment and development include, without limitation, those described in: Antica et al., Blood 84:111-117, 1994; Fine et al., Cellular Immunology 155:111-122, 1994; Galy et al., Blood 85:2770-2778, 1995; Toki et al., Proc. Nat. Acad Sci. USA 88:7548-7551, 1991.

Hematopoiesis Regulating Activity

A protein of the present invention may be useful in regulation of hematopoiesis and, consequently, in the treatment of myeloid or lymphoid cell deficiencies. Even marginal biological activity in support of colony forming cells or of factor-dependent cell lines indicates involvement in regulating hematopoiesis, e.g. in supporting the growth and proliferation of erythroid progenitor cells alone or in combination with other cytokines, thereby indicating utility, for example, in treating various anemias or for use in conjunction with irradiation/chemotherapy to stimulate the production of erythroid precursors and/or erythroid cells; in supporting the growth and proliferation of myeloid cells such as granulocytes and monocytes/macrophages (i.e., traditional CSF activity) useful, for example, in conjunction with chemotherapy to prevent or treat consequent myelo-suppression; in supporting the growth and proliferation of megakaryocytes and consequently of platelets thereby allowing prevention or treatment of various platelet disorders such as thrombocytopenia, and generally for use in place of or complimentary to platelet transfusions; and/or in supporting the growth and proliferation of hematopoietic stem cells which are capable of maturing to any and all of the above-mentioned hematopoietic cells and therefore find therapeutic utility in various stem cell disorders (such as those usually treated with transplantation, including, without limitation, aplastic anemia and paroxysmal nocturnal hemoglobinuria), as well as in repopulating the stem cell compartment post irradiation/chemotherapy, either in-vivo or ex-vivo (i.e., in conjunction with bone marrow transplantation or with peripheral progenitor cell transplantation (homologous or heterologous)) as normal cells or genetically manipulated for gene therapy.

The activity of a protein of the invention may, among other means, be measured by the following methods:

Suitable assays for proliferation and differentiation of various hematopoietic lines are cited above.

Assays for embryonic stem cell differentiation (which will identify, among others, proteins that influence embryonic differentiation hematopoiesis) include, without limitation, those described in: Johansson et al. Cellular Biology 15:141-151, 1995; Keller et al., Molecular and Cellular Biology 13:473-486, 1993; McClanahan et al., Blood 81:2903-2915, 1993.

Assays for stem cell survival and differentiation (which will identify, among others, proteins that regulate lympho-hematopoiesis) include, without limitation, those described in: Methylcellulose colony forming assays, Freshney, M. G. In Culture of Hematopoietic Cells. R. I. Freshney, et al. eds. Vol pp. 265-268, Wiley-Liss, Inc., New York, N.Y. 1994; Hirayama et al., Proc. Natl. Acad. Sci. USA 89:5907-5911, 1992; Primitive hematopoietic colony forming cells with high proliferative potential, McNiece, I. K. and Briddell, R. A. In Culture of Hematopoietic Cells. R. I. Freshney, et al. eds. Vol pp. 23-39, Wiley-Liss, Inc., New York, N.Y. 1994; Neben et al., Experimental Hematology 22:353-359, 1994; Cobblestone area forming cell assay, Ploemacher, R. E. In Culture of Hematopoietic Cells. R. I. Freshney, et al. eds. Vol pp. 1-21, Wiley-Liss, Inc., New York, N.Y. 1994; Long term bone marrow cultures in the presence of stromal cells, Spooncer, E., Dexter, M. and Allen, T. In Culture of Hematopoietic Cells. R. I. Freshney, et al. eds. Vol pp. 163-179, Wiley-Liss, Inc., New York, N.Y. 1994; Long term culture initiating cell assay, Sutherland, H. J. In Culture of Hematopoietic Cells. R. I. Freshney, et al. eds. Vol pp. 139-162, Wiley-Liss, Inc., New York, N.Y. 1994.

Tissue Growth Activity

A protein of the present invention also may have utility in compositions used for bone, cartilage, tendon, ligament and/or nerve tissue growth or regeneration, as well as for wound healing and tissue repair and replacement, and in the treatment of burns, incisions and ulcers.

A protein of the present invention, which induces cartilage and/or bone growth in circumstances where bone is not normally formed, has application in the healing of bone fractures and cartilage damage or defects in humans and other animals. Such a preparation employing a protein of the invention may have prophylactic use in closed as well as open fracture reduction and also in the improved fixation of artificial joints. De novo bone formation induced by an osteogenic agent contributes to the repair of congenital, trauma induced, or oncologic resection induced craniofacial defects, and also is useful in cosmetic plastic surgery.

A protein of this invention may also be used in the treatment of periodontal disease, and in other tooth repair processes. Such agents may provide an environment to attract bone-forming cells, stimulate growth of bone-forming cells or induce differentiation of progenitors of bone-forming cells. A protein of the invention may also be useful in the treatment of osteoporosis or osteoarthritis, such as through stimulation of bone and/or cartilage repair or by blocking inflammation or processes of tissue destruction (collagenase activity, osteoclast activity, etc.) mediated by inflammatory processes.

Another category of tissue regeneration activity that may be attributable to the protein of the present invention is tendon/ligament formation. A protein of the present invention, which induces tendon/ligament-like tissue or other tissue formation in circumstances where such tissue is not normally formed, has application in the healing of tendon or ligament tears, deformities and other tendon or ligament defects in humans and other animals. Such a preparation employing a tendon/ligament-like tissue inducing protein may have prophylactic use in preventing damage to tendon or ligament tissue, as well as use in the improved fixation of tendon or ligament to bone or other tissues, and in repairing defects to tendon or ligament tissue. De novo tendon/ligament-like tissue formation induced by a composition of the present invention contributes to the repair of congenital, trauma induced, or other tendon or ligament defects of other origin, and is also useful in cosmetic plastic surgery for attachment or repair of tendons or ligaments. The compositions of the present invention may provide an environment to attract tendon- or ligament-forming cells, stimulate growth of tendon- or ligament-forming cells, induce differentiation of progenitors of tendon- or ligament-forming cells, or induce growth of tendon/ligament cells or progenitors ex vivo for return in vivo to effect tissue repair. The compositions of the invention may also be useful in the treatment of tendinitis, carpal tunnel syndrome and other tendon or ligament defects. The compositions may also include an appropriate matrix and/or sequestering agent as a carrier as is well known in the art.

The protein of the present invention may also be useful for proliferation of neural cells and for regeneration of nerve and brain tissue, i.e. for the treatment of central and peripheral nervous system diseases and neuropathies, as well as mechanical and traumatic disorders, which involve degeneration, death or trauma to neural cells or nerve tissue. More specifically, a protein may be used in the treatment of diseases of the peripheral nervous system, such as peripheral nerve injuries, peripheral neuropathy and localized neuropathies, and central nervous system diseases, such as Alzheimer's, Parkinson's disease, Huntington's disease, amyotrophic lateral sclerosis, and Shy-Drager syndrome. Further conditions which may be treated in accordance with the present invention include mechanical and traumatic disorders, such as spinal cord disorders, head trauma and cerebrovascular diseases such as stroke. Peripheral neuropathies resulting from chemotherapy or other medical therapies may also be treatable using a protein of the invention.

Proteins of the invention may also be useful to promote better or faster closure of non-healing wounds, including without limitation pressure ulcers, ulcers associated with vascular insufficiency, surgical and traumatic wounds, and the like.

It is expected that a protein of the present invention may also exhibit activity for generation or regeneration of other tissues, such as organs (including, for example, pancreas, liver, intestine, kidney, skin, endothelium), muscle (smooth, skeletal or cardiac) and vascular (including vascular endothelium) tissue, or for promoting the growth of cells comprising such tissues. Part of the desired effects may be by inhibition or modulation of fibrotic scarring to allow normal tissue to regenerate. A protein of the invention may also exhibit angiogenic activity.

A protein of the present invention may also be useful for gut protection or regeneration and treatment of lung or liver fibrosis, reperfusion injury in various tissues, and conditions resulting from systemic cytokine damage.

A protein of the present invention may also be useful for promoting or inhibiting differentiation of tissues described above from precursor tissues or cells; or for inhibiting the growth of tissues described above.

The activity of a protein of the invention may, among other means, be measured by the following methods:

Assays for tissue generation activity include, without limitation, those described in: International Patent Publication No. WO95/16035 (bone, cartilage, tendon); International Patent Publication No. WO95/05846 (nerve, neuronal); International Patent Publication No. WO91/07491 (skin, endothelium).

Assays for wound healing activity include, without limitation, those described in: Winter, Epidermal Wound Healing, pps. 71-112 (Maibach, H I and Rovee, D T, eds.), Year Book Medical Publishers, Inc., Chicago, as modified by Eaglstein and Mertz, J. Invest. Dermatol 71:382-84 (1978).

Activin/Inhibin Activity

A protein of the present invention may also exhibit activin- or inhibin-related activities. Inhibins are characterized by their ability to inhibit the release of follicle stimulating hormone (FSH), while activins and are characterized by their ability to stimulate the release of follicle stimulating hormone (FSH). Thus, a protein of the present invention, alone or in heterodimers with a member of the inhibin a family, may be useful as a contraceptive based on the ability of inhibins to decrease fertility in female mammals and decrease spermatogenesis in male mammals. Administration of sufficient amounts of other inhibins can induce infertility in these mammals. Alternatively, the protein of the invention, as a homodimer or as a heterodimer with other protein subunits of the inhibin-β group, may be useful as a fertility inducing therapeutic, based upon the ability of activin molecules in stimulating FSH release from cells of the anterior pituitary. See, for example, U.S. Pat. No. 4,798,885. A protein of the invention may also be useful for advancement of the onset of fertility in sexually immature mammals, so as to increase the lifetime reproductive performance of domestic animals such as cows, sheep and pigs.

The activity of a protein of the invention may, among other means, be measured by the following methods:

Assays for activin/inhibin activity include, without limitation, those described in: Vale et al., Endocrinology 91:562-572, 1972; Ling et al., Nature 321:779-782, 1986; Vale et al., Nature 321:776-779, 1986; Mason et al., Nature 318:659-663, 1985; Forage et al., Proc. Natl. Acad. Sci. USA 83:3091-3095, 1986.

Chemotactic/Chemokinetic Activity

A protein of the present invention may have chemotactic or chemokinetic activity (e.g., act as a chemokine) for mammalian cells, including, for example, monocytes, fibroblasts, neutrophils, T-cells, mast cells, eosinophils, epithelial and/or endothelial cells. Chemotactic and chemokinetic proteins can be used to mobilize or attract a desired cell population to a desired site of action. Chemotactic or chemokinetic proteins provide particular advantages in treatment of wounds and other trauma to tissues, as well as in treatment of localized infections. For example, attraction of lymphocytes, monocytes or neutrophils to tumors or sites of infection may result in improved immune responses against the tumor or infecting agent.

A protein or peptide has chemotactic activity for a particular cell population if it can stimulate, directly or indirectly, the directed orientation or movement of such cell population. Preferably, the protein or peptide has the ability to directly stimulate directed movement of cells. Whether a particular protein has chemotactic activity for a population of cells can be readily determined by employing such protein or peptide in any known assay for cell chemotaxis.

The activity of a protein of the invention may, among other means, be measured by the following methods:

Assays for chemotactic activity (which will identify proteins that induce or prevent chemotaxis) consist of assays that measure the ability of a protein to induce the migration of cells across a membrane as well as the ability of a protein to induce the adhesion of one cell population to another cell population. Suitable assays for movement and adhesion include, without limitation, those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 6.12, Measurement of alpha and beta Chemokines 6.12.1-6.12.28; Taub et al. J. Clin. Invest. 95:1370-1376, 1995; Lind et al. APMIS 103:140-146, 1995; Muller et al Eur. J. Immunol. 25: 1744-1748; Gruber et al. J. of Immunol. 152:5860-5867, 1994; Johnston et al. J. of Immunol. 153: 1762-1768, 1994.

Hemostatic and Thrombolytic Activity

A protein of the invention may also exhibit hemostatic or thrombolytic activity. As a result, such a protein is expected to bc useful in treatment of various coagulation disorders (including hereditary disorders, such as hemophilias) or to enhance coagulation and other hemostatic events in treating wounds resulting from trauma, surgery or other causes. A protein of the invention may also be useful for dissolving or inhibiting formation of thromboses and for treatment and prevention of conditions resulting therefrom (such as, for example, infarction of cardiac and central nervous system vessels (e.g., stroke).

The activity of a protein of the invention may, among other means, be measured by the following methods:

Assay for hemostatic and thrombolytic activity include, without limitation, those described in: Linet et al., J. Clin. Pharmacol. 26:131-140, 1986; Burdick et al., Thrombosis Res. 45:413-419, 1987; Humphrey et al., Fibrinolysis 5:71-79 (1991); Schaub, Prostaglandins 35:467-474, 1988.

Receptor/Ligand Activity

A protein of the present invention may also demonstrate activity as receptors, receptor ligands or inhibitors or agonists of receptor/ligand interactions. Examples of such receptors and ligands include, without limitation, cytokine receptors and their ligands, receptor kinases and their ligands, receptor phosphatases and their ligands, receptors involved in cell-cell interactions and their ligands (including without limitation, cellular adhesion molecules (such as selecting, integrins and their ligands) and receptor/ligand pairs involved in antigen presentation, antigen recognition and development of cellular and humoral immune responses). Receptors and ligands are also useful for screening of potential peptide or small molecule inhibitors of the relevant receptor/ligand interaction. A protein of the present invention (including, without limitation, fragments of receptors and ligands) may themselves be useful as inhibitors of receptor/ligand interactions.

The activity of a protein of the invention may, among other means, be measured by the following methods:

Suitable assays for receptor-ligand activity include without limitation those described in: Current Protocols in Immunology, Ed by J. E. Coligan, A. M. Kruisbeek, D. H. Margulies, E. M. Shevach, W. Strober, Pub. Greene Publishing Associates and Wiley-Interscience (Chapter 7.28, Measurement of Cellular Adhesion under static conditions 7.28.1-7.28.22), Takai et al., Proc. Natl. Acad. Sci. USA 84:6864-6868, 1987: Bierer et al., J. Exp. Med. 168:1145-1156, 1988; Rosenstein et al., J. Exp. Med. 169:149-160 1989; Stoltenborg et al., J. Immunol. Methods 175:59-68, 1994; Stitt et al., Cell 80:661-670, 1995.

Anti-Inflammatory Activity

Proteins of the present invention may also exhibit anti-inflammatory activity. The anti-inflammatory activity may be achieved by providing a stimulus to cells involved in the inflammatory response, by inhibiting or promoting cell-cell interactions (such as, for example, cell adhesion), by inhibiting or promoting chemotaxis of cells involved in the inflammatory process, inhibiting or promoting cell extravasation, or by stimulating or suppressing production of other factors which more directly inhibit or promote an inflammatory response. Proteins exhibiting such activities can be used to treat inflammatory conditions including chronic or acute conditions), including without limitation inflammation associated with infection (such as septic shock, sepsis or systemic inflammatory response syndrome (SIRS)), ischemia-reperfusion injury, endotoxin lethality, arthritis, complement-mediated hyperacute rejection, nephritis, cytokine or chemokine-induced lung injury, inflammatory bowel disease, Crohn's disease or resulting from over production of cytokines such as TNF or IL-1. Proteins of the invention may also be useful to treat anaphylaxis and hypersensitivity to an antigenic substance or material.

Cadherin/Tumor Invasion Suppressor Activity

Cadherins are calcium-dependent adhesion molecules that appear to play major roles during development, particularly in defining specific cell types. Loss or alteration of normal cadherin expression can lead to changes in cell adhesion properties linked to tumor growth and metastasis. Cadherin malfunction is also implicated in other human diseases, such as pemphigus vulgaris and pemphigus foliaceus (auto-immune blistering skin diseases), Crohn's disease, and some developmental abnormalities.

The cadherin superfamily includes well over forty members, each with a distinct pattern of expression. All members of the superfamily have in common conserved extracellular repeats (cadherin domains), but structural differences are found in other parts of the molecule. The cadherin domains bind calcium to form their tertiary structure and thus calcium is required to mediate their adhesion. Only a few amino acids in the first cadherin domain provide the basis for homophilic adhesion; modification of this recognition site can change the specificity of a cadherin so that instead of recognizing only itself, the mutant molecule can now also bind to a different cadherin. In addition, some cadherins engage in heterophilic adhesion with other cadherins.

E-cadherin, one member of the cadherin superfamily, is expressed in epithelial cell types. Pathologically, if E-cadherin expression is lost in a tumor, the malignant cells become invasive and the cancer metastasizes. Transfection of cancer cell lines with polynucleotides expressing E-cadherin has reversed cancer-associated changes by returning altered cell shapes to normal, restoring cells' adhesiveness to each other and to their substrate, decreasing the cell growth rate, and drastically reducing anchorage-independent cell growth. Thus, reintroducing E-cadherin expression reverts carcinomas to a less advanced stage. It is likely that other cadherins have the same invasion suppressor role in carcinomas derived from other tissue types. Therefore, proteins of the present invention with cadherin activity, and polynucleotides of the present invention encoding such proteins, can be used to treat cancer. Introducing such proteins or polynucleotides into cancer cells can reduce or eliminate the cancerous changes observed in these cells by providing normal cadherin expression.

Cancer cells have also been shown to express cadherins of a different tissue type than their origin, thus allowing these cells to invade and metastasize in a different tissue in the body. Proteins of the present invention with cadherin activity, and polynucleotides of the present invention encoding such proteins, can be substituted in these cells for the inappropriately expressed cadherins, restoring normal cell adhesive properties and reducing or eliminating the tendency of the cells to metastasize.

Additionally, proteins of the present invention with cadherin activity, and polynucleotides of the present invention encoding such proteins, can used to generate antibodies recognizing and binding to cadherins. Such antibodies can be used to block the adhesion of inappropriately expressed tumor-cell cadherins, preventing the cells from forming a tumor elsewhere. Such an anti-cadherin antibody can also be used as a marker for the grade, pathological type, and prognosis of a cancer, i.e. the more progressed the cancer, the less cadherin expression there will be, and this decrease in cadherin expression can be detected by the use of a cadherin-binding antibody.

Fragments of proteins of the present invention with cadherin activity, preferably a polypeptide comprising a decapeptide of the cadherin recognition site, and poly-nucleotides of the present invention encoding such protein fragments, can also be used to block cadherin function by binding to cadherins and preventing them from binding in ways that produce undesirable effects. Additionally, fragments of proteins of the present invention with cadherin activity, preferably truncated soluble cadherin fragments which have been found to be stable in the circulation of cancer patients, and polynucleotides encoding such protein fragments, can be used to disturb proper cell-cell adhesion.

Assays for cadherin adhesive and invasive suppressor activity include, without limitation, those described in: Hortsch et al. J Biol Chem 270 (32): 18809-18817, 1995; Miyaki et al. Oncogene 11: 2547-2552, 1995; Ozawa et al. Cell 63: 1033-1038, 1990.

Tumor Inhibition Activity

In addition to the activities described above for immunological treatment or prevention of tumors, a protein of the invention may exhibit other anti-tumor activities. A protein may inhibit tumor growth directly or indirectly (such as, for example, via antibody-dependent cell-mediated cytotoxicity (ADCC)). A protein may exhibit its tumor inhibitory activity by acting on tumor tissue or tumor precursor tissue, by inhibiting formation of tissues necessary to support tumor growth (such as, for example, by inhibiting angiogenesis), by causing production of other factors, agents or cell types which inhibit tumor growth, or by suppressing, eliminating or inhibiting factors, agents or cell types which promote tumor growth.

Other Activities

A protein of the invention may also exhibit one or more of the following additional activities or effects: inhibiting the growth, infection or function of, or killing, infectious agents, including, without limitation, bacteria, viruses, fungi and other parasites; effecting (suppressing or enhancing) bodily characteristics, including, without limitation, height, weight, hair color, eye color, skin, fat to lean ratio or other tissue pigmentation, or organ or body part size or shape (such as, for example, breast augmentation or diminution, change in bone form or shape); effecting biorhythms or caricadic cycles or rhythms; effecting the fertility of male or female subjects; effecting the metabolism, catabolism, anabolism, processing, utilization, storage or elimination of dietary fat, lipid, protein, carbohydrate, vitamins, minerals, cofactors or other nutritional factors or component(s); effecting behavioral characteristics, including, without limitation, appetite, libido, stress, cognition (including cognitive disorders), depression (including depressive disorders) and violent behaviors; providing analgesic effects or other pain reducing effects; promoting differentiation and growth of embryonic stem cells in lineages other than hematopoietic lineages; hormonal or endocrine activity; in the case of enzymes, correcting deficiencies of the enzyme and treating deficiency-related diseases; treatment of hyperproliferative disorders (such as, for example, psoriasis); immunoglobulin-like activity (such as, for example, the ability to bind antigens or complement); and the ability to act as an antigen in a vaccine composition to raise an immune response against such protein or another material or entity which is cross-reactive with such protein.

Administration and Dosing

A protein of the present invention (from whatever source derived, including without limitation from recombinant and non-recombinant sources) may be used in a pharmaceutical composition when combined with a pharmaceutically acceptable carrier. Such a composition may also contain (in addition to protein and a carrier) diluents, fillers, salts, buffers, stabilizers, solubilizers, and other materials well known in the art. The term “pharmaceutically acceptable” means a non-toxic material that does not interfere with the effectiveness of the biological activity of the active ingredient(s). The characteristics of the carrier will depend on the route of administration. The pharmaceutical composition of the invention may also contain cytokines, lymphokines, or other hematopoietic factors such as M-CSF, GM-CSF, TNF, IL-1, IL-2, IL-3, IL-4, IL-5, IL-6, IL-7, IL-8, IL-9, IL-10, IL-11, IL-12, IL-13, IL-14, IL-15, IFN, TNF0, TNF1, TNF2, G-CSF, Meg-CSF, thrombopoietin, stem cell factor, and erythropoietin. The pharmaceutical composition may further contain other agents which either enhance the activity of the protein or compliment its activity or use in treatment. Such additional factors and/or agents may be included in the pharmaceutical composition to produce a synergistic effect with protein of the invention, or to minimize side effects. Conversely, protein of the present invention may be included in formulations of the particular cytokine, lymphokine, other hematopoietic factor, thrombolytic or anti-thrombotic factor, or anti-inflammatory agent to minimize side effects of the cytokine, lymphokine, other hematopoietic factor, thrombolytic or anti-thrombotic factor, or anti-inflammatory agent.

A protein of the present invention may be active in multimers (e.g., heterodimers or homodimers) or complexes with itself or other proteins. As a result, pharmaceutical compositions of the invention may comprise a protein of the invention in such multimeric or complexed form.

The pharmaceutical composition of the invention may be in the form of a complex of the protein(s) of present invention along with protein or peptide antigens. The protein and/or peptide antigen will deliver a stimulatory signal to both B and T lymphocytes. B lymphocytes will respond to antigen through their surface immunoglobulin receptor. T lymphocytes will respond to antigen through the T cell receptor (TCR) following presentation of the antigen by MHC proteins. MHC and structurally related proteins including those encoded by class I and class II MHC genes on host cells will serve to present the peptide antigen(s) to T lymphocytes. The antigen components could also be supplied as purified MHC-peptide complexes alone or with co-stimulatory molecules that can directly signal T cells. Alternatively antibodies able to bind surface immunolgobulin and other molecules on B cells as well as antibodies able to bind the TCR and other molecules on T cells can be combined with the pharmaceutical composition of the invention.

The pharmaceutical composition of the invention may be in the form of a liposome in which protein of the present invention is combined, in addition to other pharmaceutically acceptable carriers, with amphipathic agents such as lipids which exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, and the like. Preparation of such liposomal formulations is within the level of skill in the art, as disclosed, for example, in U.S. Pat. No. 4,235,871; U.S. Pat. No. 4,501,728; U.S. Pat. No. 4,837,028; and U.S. Pat. No. 4,737,323, all of which are incorporated herein by reference.

As used herein, the term “therapeutically effective amount” means the total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

In practicing the method of treatment or use of the present invention, a therapeutically effective amount of protein of the present invention is administered to a mammal having a condition to be treated. Protein of the present invention may be administered in accordance with the method of the invention either alone or in combination with other therapies such as treatments employing cytokines, lymphokines or other hematopoietic factors. When co-administered with one or more cytokines, lymphokines or other hematopoietic factors, protein of the present invention may be administered either simultaneously with the cytokine(s), lymphokine(s), other hematopoietic factor(s), thrombolytic or anti-thrombotic factors, or sequentially. If administered sequentially, the attending physician will decide on the appropriate sequence of administering protein of the present invention in combination with cytokine(s), lymphokine(s), other hematopoietic factor(s), thrombolytic or anti-thrombotic factors.

Administration of protein of the present invention used in the pharmaceutical composition or to practice the method of the present invention can be carried out in a variety of conventional ways, such as oral ingestion, inhalation, topical application or cutaneous, subcutaneous, intraperitoneal, parenteral or intravenous injection. Intravenous administration to the patient is preferred.

When a therapeutically effective amount of protein of the present invention is administered orally, protein of the present invention will be in the form of a tablet, capsule, powder, solution or elixir. When administered in tablet form, the pharmaceutical composition of the invention may additionally contain a solid carrier such as a gelatin or an adjuvant. The tablet, capsule, and powder contain from about 5 to 95% protein of the present invention, and preferably from about 25 to 90% protein of the present invention. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol or polyethylene glycol. When administered in liquid form, the pharmaceutical composition contains from about 0.5 to 90% by weight of protein of the present invention, and preferably from about 1 to 50% protein of the present invention.

When a therapeutically effective amount of protein of the present invention is administered by intravenous, cutaneous or subcutaneous injection, protein of the present invention will be in the form of a pyrogen-free, parenterally acceptable aqueous solution. The preparation of such parenterally acceptable protein solutions, having due regard to pH, isotonicity, stability, and the like, is within the skill in the art. A preferred pharmaceutical composition for intravenous, cutaneous, or subcutaneous injection should contain, in addition to protein of the present invention, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art.

The amount of protein of the present invention in the pharmaceutical composition of the present invention will depend upon the nature and severity of the condition being treated, and on the nature of prior treatments which the patient has undergone. Ultimately, the attending physician will decide the amount of protein of the present invention with which to treat each individual patient. Initially, the attending physician will administer low doses of protein of the present invention and observe the patient's response. Larger doses of protein of the present invention may be administered until the optimal therapeutic effect is obtained for the patient, and at that point the dosage is not increased further. It is contemplated that the various pharmaceutical compositions used to practice the method of the present invention should contain about 0.01 μg to about 100 mg (preferably about 0.1 ng to about 10 mg, more preferably about 0.1 μg to about 1 mg) of protein of the present invention per kg body weight.

The duration of intravenous therapy using the pharmaceutical composition of the present invention will vary, depending on the severity of the disease being treated and the condition and potential idiosyncratic response of each individual patient. It is contemplated that the duration of each application of the protein of the present invention will be in the range of 12 to 24 hours of continuous intravenous administration. Ultimately the attending physician will decide on the appropriate duration of intravenous therapy using the pharmaceutical composition of the present invention.

Protein of the invention may also be used to immunize animals to obtain polyclonal and monoclonal antibodies which specifically react with the protein. As used herein, the term “antibody” includes without limitation a polyclonal antibody, a monoclonal antibody, a chimeric antibody, a single-chain antibody, a CDR-grafted antibody, a humanized antibody, or fragments thereof which bind to the indicated protein. Such term also includes any other species derived from an antibody or antibody sequence which is capable of binding the indicated protein.

Antibodies to a particular protein can be produced by methods well known to those skilled in the art. For example, monoclonal antibodies can be produced by generation of antibody-producing hybridomas in accordance with known methods (see for example, Goding, 1983, Monoclonal antibodies: principles and practice, Academic Press Inc. New York; and Yokoyama, 1992, “Production of Monoclonal Antibodies” in Current Protocols in Immunology, Unit 2.5. Greene Publishing Assoc. and John Wiley & Sons). Polyclonal sera and antibodies can be produced by inoculation of a mammalian subject with the relevant protein or fragments thereof in accordance with known methods. Fragments of antibodies, receptors, or other reactive peptides can be produced from the corresponding antibodies by cleavage of and collection of the desired fragments in accordance with known methods (see for example, Goding, supra; and Andrew et al., 1992, “Fragmentation of Immunoglobulins” in Current Protocols in Immunology, Unit 2.8, Greene Publishing Assoc. and John Wiley & Sons). Chimeric antibodies and single chain antibodies can also be produced in accordance with known recombinant methods (see for example, U.S. Pat. Nos. 5,169,939, 5,194,594, and 5,576,184). Humanized antibodies can also be made from corresponding murine antibodies in accordance with well known methods (see for example; U.S. Pat. Nos. 5,530,101, 5,585,089, and 5,693,762). Additionally, human antibodies may be produced in non-human animals such as mice that have been genetically altered to express human antibody molecules (see for example Fishwild et al., 1996, Nature Biotechnology 14: 845-851; Mendez et al., 1997, Nature Genetics 15: 146-156 (erratum Nature Genetics 16: 410); and U.S. Pat. Nos. 5,877,397 and 5,625,126). Such antibodies may be obtained using either the entire protein or fragments thereof as an immunogen. The peptide immunogens additionally may contain a cysteine residue at the carboxyl terminus, and are conjugated to a hapten such as keyhole limpet hemocyanin (KLH). Methods for synthesizing such peptides are known in the art, for example, as in R. P. Merrifield, J. Amer. Chem. Soc. 85, 2149-2154 (1963); J. L. Krstenansky, et al., FEBS Lett. 211, 10 (1987).

Monoclonal antibodies binding to the protein of the invention may be useful diagnostic agents for the immunodetection of the protein. Neutralizing monoclonal antibodies binding to the protein may also be useful therapeutics for both conditions associated with the protein and also in the treatment of some forms of cancer where abnormal expression of the protein is involved. In the case of cancerous cells or leukemic cells, neutralizing monoclonal antibodies against the protein may be useful in detecting and preventing the metastatic spread of the cancerous cells, which may be mediated by the protein.

For compositions of the present invention which are useful for bone, cartilage, tendon or ligament regeneration, the therapeutic method includes administering the composition topically, systematically, or locally as an implant or device. When administered, the therapeutic composition for use in this invention is, of course, in a C pyrogen-free, physiologically acceptable form. Further, the composition may desirably be encapsulated or injected in a viscous form for delivery to the site of bone, cartilage or tissue damage. Topical administration may be suitable for wound healing and tissue repair. Therapeutically useful agents other than a protein of the invention which may also optionally be included in the composition as described above, may alternatively or additionally, be administered simultaneously or sequentially with the composition in the methods of the invention. Preferably for bone and/or cartilage formation, the composition would include a matrix capable of delivering the protein-containing composition to the site of bone and/or cartilage damage, providing a structure for the developing bone and cartilage and optimally capable of being resorbed into the body. Such matrices may be formed of materials presently in use for other implanted medical applications.

The choice of matrix material is based on biocompatibility, biodegradability, mechanical properties, cosmetic appearance and interface properties. The particular application of the compositions will define the appropriate formulation. Potential matrices for the compositions may be biodegradable and chemically defined calcium sulfate, tricalciumphosphate, hydroxyapatite, polylactic acid, polyglycolic acid and polyanhydrides. Other potential materials are biodegradable and biologically well-defined, such as bone or dermal collagen. Further matrices are comprised of pure proteins or extracellular matrix components. Other potential matrices are nonbiodegradable and chemically defined, such as sintered hydroxapatite, bioglass, aluminates, or other ceramics. Matrices may be comprised of combinations of any of the above mentioned types of material, such as polylactic acid and hydroxyapatite or collagen and tricalciumphosphate. The bioceramics may be altered in composition, such as in calcium-aluminate-phosphate and processing to alter pore size, particle size, particle shape, and biodegradability.

Presently preferred is a 50:50 (mole weight) copolymer of lactic acid and glycolic acid in the form of porous particles having diameters ranging from 150 to 800 microns. In some applications, it will be useful to utilize a sequestering agent, such as carboxymethyl cellulose or autologous blood clot, to prevent the protein compositions from disassociating from the matrix.

A preferred family of sequestering agents is cellulosic materials such as alkylcelluloses (including hydroxyalkylcelluloses), including methylcellulose, ethylcellulose, hydroxyethylcellulose, hydroxypropylcellulose, hydroxypropyl-methylcellulose, and carboxymethylcellulose, the most preferred being cationic salts of carboxymethylcellulose (CMC). Other preferred sequestering agents include hyaluronic acid, sodium alginate, poly(ethylene glycol), polyoxyethylene oxide, carboxyvinyl polymer and poly(vinyl alcohol). The amount of sequestering agent useful herein is 0.5-20 wt %, preferably 1-10 wt % based on total formulation weight, which represents the amount necessary to prevent desorbtion of the protein from the polymer matrix and to provide appropriate handling of the composition, yet not so much that the progenitor cells are prevented from infiltrating the matrix, thereby providing the protein the opportunity to assist the osteogenic activity of the progenitor cells.

In further compositions, proteins of the invention may be combined with other agents beneficial to the treatment of the bone and/or cartilage defect, wound, or tissue in question. These agents include various growth factors such as epidermal growth factor (EGF), platelet derived growth factor (PDGF), transforming growth factors (TGF-α and TGF-β), and insulin-like growth factor (IGF).

The therapeutic compositions are also presently valuable for veterinary applications. Particularly domestic animals and thoroughbred horses, in addition to humans, are desired patients for such treatment with proteins of the present invention.

The dosage regimen of a protein-containing pharmaceutical composition to be used in tissue regeneration will be determined by the attending physician considering various factors which modify the action of the proteins, e.g., amount of tissue weight desired to be formed, the site of damage, the condition of the damaged tissue, the size of a wound, type of damaged tissue (e.g., bone), the patient's age, sex, and diet, the severity of any infection, time of administration and other clinical factors. The dosage may vary with the type of matrix used in the reconstitution and with inclusion of other proteins in the pharmaceutical composition. For example, the addition of other known growth factors, such as IGF I (insulin like growth factor I), to the final composition, may also effect the dosage. Progress can be monitored by periodic assessment of tissue/bone growth and/or repair, for example, X-rays, histomorphometric determinations and tetracycline labeling.

Polynucleotides of the present invention can also be used for gene therapy. Such polynucleotides can be introduced either in vivo or ex vivo into cells for expression in a mammalian subject. Polynucleotides of the invention may also be administered by other known methods for introduction of nucleic acid into a cell or organism (including, without limitation, in the form of viral vectors or naked DNA).

Cells may also be cultured ex vivo in the presence of proteins of the present invention in order to proliferate or to produce a desired effect on or activity in such cells. Treated cells can then be introduced in vivo for therapeutic purposes.

Patent and literature references cited herein are incorporated by reference as if fully set forth.

1-13. (canceled)

14. An isolated polynucleotide selected from the group consisting of:
(a) a nucleotide sequence of SEQ ID NO: 209;
(b) a nucleotide sequence of SEQ ID NO: 209 from nucleotide 199 to nucleotide 1155;
(c) a nucleotide sequence of SEQ ID NO: 209 from nucleotide 304 to nucleotide 1155;
(d) a nucleotide sequence encoding a protein comprising the amino acid sequence of SEQ ID NO:210;
(e) a nucleotide sequence encoding a protein comprising a fragment of the amino acid sequence of SEQ ID NO:210, the fragment comprising eight contiguous amino acids of SEQ ID NO:210.

15. The polynucleotide of claim 14 wherein said polynucleotide is operably linked to at least one expression control sequence.

16. A host cell transformed with the polynucleotide of claim 15.

17. The host cell of claim 16, wherein said cell is a mammalian cell.

18. A process for producing a protein encoded by the polynucleotide of claim 15, which process comprises:
(a) growing a culture of a host cell in a suitable culture medium, wherein the host cell has been transformed with said polynucleotide; and
(b) purifying said protein from the culture.

19. A protein produced according to the process of claim 18.

20. A protein comprising an amino acid sequence selected from the group consisting of:
(a) the amino acid sequence of SEQ ID NO:210;
(b) the amino acid sequence of SEQ ID NO:210 from amino acid 19 to amino acid 84;
(c) a fragment of the amino acid sequence of SEQ ID NO:210, the fragment comprising eight contiguous amino acids of SEQ ID NO:210.

21. The protein of claim 20, wherein said protein comprises the amino acid sequence of SEQ ID NO:210.

22. A composition comprising the protein of claim 21 and a pharmaceutically acceptable carrier.


SEQUENCE LISTING (321 sequences)

SEQ ID NO: 1 - DNA, 812 bp, Homo sapiens
aggaagatacattcacagaagagcttcctgcacaaagtaagccaccagcgcaacatgacagtgaagaccctgcatggcccagccatggtcaagtacttgctgctgtcgatattggggcttgcctttctgagtgaggcggcagctcggaaaatccccaaagtaggacatacttttttccaaaagcctgagagttgcccgcctgtgccaggaggtagtatgaagcttgacattggcatcatcaatgaaaaccagcgcgtttccatgtcacgtaacatcgagagccgctccacctccccctggaattacactgtcacttgggaccccaaccggtacccctcggaagttgtacaggcccagtgtaggaacttgggctgcatcaatgctcaaggaaaggaagacatctccatgaattccgttcccatccagcaagagaccctggtcggccggaggaagcaccaaggctgctctgtttctttccagttggagaaggtgctggtgactgttggctgcacctgcgtcacccctgtcatccaccatgtgcagtaagaggtgcatatccactcagctgaagaagctgtagaaatgccactccttacccagtgctctgcaacaagtcctgtctgacccccaattccctccacttcacaggactcttaataagacctgcacggatggaaacagaaaatattcacaatgtatgtgtgtatgtactacactttatatttgatatctaaaatgttaggagaaaaattaatatattcagtgctaatataataaagtattaataatttaaaaataaaaaaaaaaaaaaa

SEQ ID NO: 2 - protein, 163 aa, Homo sapiens
MTVKTLHGPAMVKYLLLSILGLAFLSEAAARKIPKVGHTFFQKPESCPPVPGGSMKLDIGIINENQRVSMSRNIESRSTSPWNYTVTWDPNRYPSEVVQAQCRNLGCINAQGKEDISMNSVPIQQETLVGRRKHQGCSVSFQLEKVLVTVGCTCVTPVIHHVQ

SEQ ID NO: 3 - DNA, 966 bp, Homo sapiens
cttccaagaagagcagcaaagctgaagtagcagcaacagcaccagcagcaacagcaaaaaacaaacatgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaaaaagttgaaagaaagaatttttaaaaatatcaaaacatatgaagtcctggaaaagggcatctgaaaaacctagaacaagtttaactgtgactactgaaatgacaagaattctacagtaggaaactgagacttttctatggttttgtgactttcaacttttgtacagttatgtgaaggatgaaaggtgggtgaaaggaccaaaaacagaaatacagtcttcctgaatgaatgacaatcagaattccactgcccaaaggagtccaacaattaaatggatttctaggaaaagctaccttaagaaaggctggttaccatcggagtttacaaagtgctttcacgttcttacttgttgtattatacattcatgcatttctaggctagagaaccttctagatttgatgcttacaactattctgttgtgactatgagaacatttctgtctctagaagttatctgtctgtattgatctttatgctatattactatctgtggttacagtggagacattgacattattactggagtcaagcccttataagtcaaaagcacctatgtgtcgtaaagcattcctcaaacatttaaaaaaaaaaaaaaaaaaaaaaaaaaaaaaaaaaaaaaaaaaaaaaaaaaaaaaaaaaa

SEQ ID NO: 4 - protein, 94 aa, Homo sapiens
MSVKGMAIALAVILCATVVQGFPMFKRGRCLCIGPGVKAVKVADIEKASIMYPSNNCDKIEVIITLKENKGQRCLNPKSKQARLIIKKVERKNF

SEQ ID NO: 5 - DNA, 1354 bp, Homo sapiens
ttctactccttccaagaagagcagcaaagctgaagtagcagcaacagcaccagcagcaacagcaaaaaacaaacatgagtgtgaagggcatggctatagccttggctgtgatattgtgtgctacagttgttcaaggcttccccatgttcaaaagaggacgctgtctttgcataggccctggggtaaaagcagtgaaagtggcagatattgagaaagcctccataatgtacccaagtaacaactgtgacaaaatagaagtgattattaccctgaaagaaaataaaggacaacgatgcctaaatcccaaatcgaagcaagcaaggcttataatcaaaaaagttgaaagaaagaatttttaaaaatatcaaaacatatgaagtcctggaaaagggcatctgaaaaacctagaacaagtttaactgtgactactgaaatgacaagaattctacagtaggaaactgagacttttctatggttttgtgactttcaacttttgtacagttatgtgaaggatgaaaggtgggtgaaaggaccaaaaacagaaatacagtcttcctgaatgaatgacaatcagaattccactgcccaaaggagtccaacaattaaatggatttctaggaaaagctaccttaagaaaggctggttaccatcggagtttacaaagtgctttcacgttcttacttgttgtattatacattcatgcatttctaggctagagaaccttctagatttgatgcttacaactattctgttgtgactatgagaacatttctgtctctagaagttatctgtctgtattgatctttatgctatattactatctgtggttacagtggagacattgacattattactggagtcaagcccttataagtcaaaagcacctatgtgtcgtaaagcattcctcaaacattttttcatgcaaatacacacttctttccccaaatatcatgtagcacatcaatatgtagggaaacattcttatgcatcatttggtttgttttataaccaattcattaaatgtaattcataaaatgtactatgaaaaaaattatacgctatgggatactggcaacagtgcacatatttcataaccaaattagcagcaccggtcttaatttgatgtttttcaacttttattcattgagatgttttgaagcaattaggatatgtgtgtttactgtactttttgttttgatccgtttgtataaatgatagcaatatcttggacacatttgaaatacaaaatgtttttgtctaccaaagaaaaatgttgaaaaataagcaaatgtatacctagcaatcacttttactttttgtaattctgtctcttagaaaaatacataatctaatt

SEQ ID NO: 6 - protein, 94 aa, Homo sapiens; identical to SEQ ID NO: 4

SEQ ID NO: 7 - DNA, 1462 bp, Homo sapiens
ctgcagaacttcatgctgcgtgggatctcccagctaccggcagtggccaccatgtcttgggtcctgctgcctgtactttggctcattgttcaaactcaagcaatagccataaagcaaacacctgaattaacgctccatgaaatagtttgtcctaaaaaacttcacattttacacaaaagagagatcaagaacaaccagacagaaaagcatggcaaagaggaaaggtatgaacctgaagttcaatatcagatgatcttaaatggagaagaaatcattctctccctacaaaaaaccaagcacctcctggggccagactacactgaaacattgtactcacccagaggagaggaaattaccacgaaacctgagaacatggaacactgttactataaaggaaacatcctaaatgaaaagaattctgttgccagcatcagtacttgtgacgggttgagaggatacttcacacatcatcaccaaagataccagataaaacctctgaaaagcacagacgagaaagaacatgccgtctttacatctaaccaggaggaacaagacccagctaaccacacatgtggtgtgaagagcactgacgggaaacaaggcccaattcgaatctctagatcactcaaaagcccagagaaagaagactttcttcgggcacagaaatacattgatctctatttggtgctggataatgccttttataagaactataatgagaatctaactctgataagaagctttgtgtttgatgtgatgaacctactcaatgtgatatataacaccatagatgttcaagtggccttggtaggtatggaaatctggtctgatggggataagataaaggtggtgcccagcgcaagcaccacgtttgacaacttcctgagatggcacagttctaacctggggaaaaagatccacgaccatgctcagcttctcagcgggattagcttcaacaatcgacgtgtgggactggcagcttcaaattccttgtgttccccatcttcggttgctgttattgaggctaaaaaaaagaataatgtggctcttgtaggagtgatgtcacatgagctgggccatgtccttggtatgcctgatgttccattcaacaccaagtgtccctctggcagttgtgtgatgaatcagtatctgagttcaaaattcccaaaggatttcagtacatcttgccgtgcacattttgaaagataccttttatctcagaaaccaaagtgcctgctgcaagcacctattcctacaaatataatgacaacaccagtgtgtgggaaccaccttctagaagtgggagaagactgtgattgtggctctcctaaggagtgtaccaatctctgctgtgaagccctaacgtgtaaactgaagcctggaactgattgcggaggagatgctccaaaccataccacagagtgaatccaaaagtctgcttcactgagatgctaccgtcgac

SEQ ID NO: 8 - protein, 470 aa, Homo sapiens
MLRGISQLPAVATMSWVLLPVLWLIVQTQAIAIKQTPELTLHEIVCPKKLHILHKREIKNNQTEKHGKEERYEPEVQYQMILNGEEIILSLQKTKHLLGPDYTETLYSPRGEEITTKPENMEHCYYKGNILNEKNSVASISTCDGLRGYFTHHHQRYQIKPLKSTDEKEHAVFTSNQEEQDPANHTCGVKSTDGKQGPIRISRSLKSPEKEDFLRAQKYIDLYLVLDNAFYKNYNENLTLIRSFVFDVMNLLNVIYNTIDVQVALVGMEIWSDGDKIKVVPSASTTFDNFLRWHSSNLGKKIHDHAQLLSGISFNNRRVGLAASNSLCSPSSVAVIEAKKKNNVALVGVMSHELGHVLGMPDVPFNTKCPSGSCVMNQYLSSKFPKDFSTSCRAHFERYLLSQKPKCLLQAPIPTNIMTTPVCGNHLLEVGEDCDCGSPKECTNLCCEALTCKLKPGTDCGGDAPNHTTE

SEQ ID NO: 9 - DNA, 2554 bp, Homo sapiens
ccaccaagcaatcctagcctgtgatggcgtttgacgtcagctgcttcttttgggtggtgctgttttctgccggctgtaaagtcatcacctcctgggatcagatgtgcattgagaaagaagccaacaaaacatataactgtgaaaatttaggtctcagtgaaatccctgacactctaccaaacacaacagaatttttggaattcagctttaattttttgcctacaattcacaatagaaccttcagcagactcatgaatcttacctttttggatttaactaggtgccagattaactggatacatgaagacacttttcaaagccatcatcaattaagcacacttgtgttaactggaaatcccctgatattcatggcagaaacatcgcttaatgggcccaagtcactgaagcatcttttcttaatccaaacgggaatatccaatctcgagtttattccagtgcacaatctggaaaacttggaaagcttgtatcttggaagcaaccatatttcctccattaagttccccaaagacttcccagcacggaatctgaaagtactggattttcagaataatgctatacactacatctctagagaagacatgaggtctctggagcaggccatcaacctaagcctgaacttcaatggcaataatgttaaaggtattgagcttggggcttttgattcaacggtcttccaaagtttgaactttggaggaactccaaatttgtctgttatattcaatggtctgcagaactctactactcagtctctctggctgggaacatttgaggacattgatgacgaagatattagttcagccatgctcaagggactctgtgaaatgtctgttgagagcctcaacctgcaggaacaccgcttctctgacatctcatccaccacatttcagtgcttcacccaactccaagaattggatctgacagcaactcacttgaaagggttaccctctgggatgaagggtctgaacttgctcaagaaattagttctcagtgtaaatcatttcgatcaattgtgtcaaatcagtgctgccaatttcccctcccttacacacctctacatcagaggcaacgtgaagaaacttcaccttggtgttggctgcttggagaaactaggaaaccttcagacacttgatttaagccataatgacatagaggcttctgactgctgcagtctgcaactcaaaaacctgtcccacttgcaaaccttaaacctgagccacaatgagcctcttggtctccagagtcaggcattcaaagaatgtcctcagctagaactcctcgatttggcatttacccgcttacacattaatgctccacaaagtcccttccaaaacctccatttccttcaggttctgaatctcacttactgcttccttgataccagcaatcagcatcttctagcaggcctaccagttctccggcatctcaacttaaaagggaatcactttcaagatgggactatcacgaagaccaacctacttcagaccgtgggcagcttggaggttctgattttgtcctcttgtggtctcctctctatagaccagcaagcattccacagcttgggaaaaatgagccatgtagacttaagccacaacagcctgacatgcgacagcattgattctcttagccatcttaagggaatctacctcaatctggctgccaacagcattaacatcatctcaccccgtctcctccctatcttgtcccagcagagcaccattaatttaagtcataaccccctggactgcacttgctcgaatattcatttcttaacatggtacaaagaaaacctgcacaaacttgaaggctcggaggagaccacgtgtgcaaacccgccatctctaaggggagttaagctatctgatgtcaagctttcctgtgggattacagccataggcattttctttctcatagtatttctattattgttggctattctgctattttttgcagttaaataccttctcaggtggaaataccaacacatttagtgctgaaggtttccagagaaagcaaataagtgtgcttagcaaaattgctctaagtgaaagaactgtcatctgctggtgaccagaccagacttttcagattgcttcctggaactgggcagggactcactgtgcttttctgagcttcttactcctgtgagtcccagagctaaagaaccttctaggcaagtacaccgaatgactcagtccagagggtcagatgctgctgtgagaggcacagagccctttccgcatgtggaagagtgggaggaagcagagggagggactgggcagggactgccggccccggagtctcccacagggaggccattccccttctactcaccgacatccctcccagcaccacacaccccgcccctgaaaggagatcatcagcccccacaatttgtcagagctgaagccagcccactacccacccccactacagcattgtgcttgggtctgggttctcagtaatgtagccatttgagaaacttacttggggacaaagtctcaatccttattttaaatgaaaaaaaaaaaaaaaa

SEQ ID NO: 10 - protein, 661 aa, Homo sapiens
MAFDVSCFFWVVLFSAGCKVITSWDQMCIEKEANKTYNCENLGLSEIPDTLPNTTEFLEFSFNFLPTIHNRTFSRLMNLTFLDLTRCQINWIHEDTFQSHHQLSTLVLTGNPLIFMAETSLNGPKSLKHLFLIQTGISNLEFIPVHNLENLESLYLGSNHISSIKFPKDFPARNLKVLDFQNNAIHYISREDMRSLEQAINLSLNFNGNNVKGIELGAFDSTVFQSLNFGGTPNLSVIFNGLQNSTTQSLWLGTFEDIDDEDISSAMLKGLCEMSVESLNLQEHRFSDISSTTFQCFTQLQELDLTATHLKGLPSGMKGLNLLKKLVLSVNHFDQLCQISAANFPSLTHLYIRGNVKKLHLGVGCLEKLGNLQTLDLSHNDIEASDCCSLQLKNLSHLQTLNLSHNEPLGLQSQAFKECPQLELLDLAFTRLHINAPQSPFQNLHFLQVLNLTYCFLDTSNQHLLAGLPVLRHLNLKGNHFQDGTITKTNLLQTVGSLEVLILSSCGLLSIDQQAFHSLGKMSHVDLSHNSLTCDSIDSLSHLKGIYLNLAANSINIISPRLLPILSQQSTINLSHNPLDCTCSNIHFLTWYKENLHKLEGSEETTCANPPSLRGVKLSDVKLSCGITAIGIFFLIVFLLLLAILLFFAVKYLLRWKYQHI

SEQ ID NO: 11 - DNA, 479 bp, Mus musculus
gtcgactcacctacagctctggtctcatcctcaactcaaccacaatcatggctcagatgatgactctgagcctccttagcctggtcctggctctctgcatcccctggacccaaggcagtgatggagggggtcaggactgctgccttaagtacagccagaagaaaattccctacagtattgtccgaggctataggaagcaagaaccaagtttaggctgtcccatcccggcaatcctgttctcaccccggaagcactctaagcctgagctatgtgcaaaccctgaggaaggctgggtgcagaacctgatgcgccgcctggaccagcctccagccccagggaaacaaagccccggctgcaggaagaaccggggaacctctaagtctggaaagaaaggaaagggctccaagggctgcaagagaactgaacagacacagccctcaagaggatagcccagtagcccgcctggagcccaggagatc

SEQ ID NO: 12 - protein, 133 aa, Mus musculus
MAQMMTLSLLSLVLALCIPWTQGSDGGGQDCCLKYSQKKIPYSIVRGYRKQEPSLGCPIPAILFSPRKHSKPELCANPEEGWVQNLMRRLDQPPAPGKQSPGCRKNRGTSKSGKKGKGSKGCKRTEQTQPSRG

SEQ ID NO: 13 - DNA, 1731 bp, Homo sapiens
cccggcaggttgccagcgtcgctacagcccagaccaaggsagaataatctccggatgagctggtggcaccgctgagcctttggtctcaccagggcttcctgttgctggcaggcggggtggagcggagctgctgggaggctgctggataggagaggggtcacggctgcggaagaggaggttcttcgggacacccgtggatggacacggcaaggaaacaccaggccaaccacagstggggataaaatagsacaaccacaccctgccgtccagcgcctcccagcctgtgccccttcctagtaccaccagcaaccatcaatcccgtctcctcctgcctcctctcctgcaatccaccccgccacgactatcgccatggcagccctgatcgcagagaacttccgcttcctgtcacttttcttcaagagcaaggatgtgatgattttcaacggcctggtggcactgggcacggtgggcagccaggagctgtyctctgtggtggccttccactgcccctgctcgccggcccggaactacctgtacgggctggcggccatcggcgtgcccgccctggtgctcttcatcattggcatcatcctcaacaaccacacctggaacctcgtggccgagtgccagcaccggaggaccaagaactgctccgccgcccccaccttcctccttctaagctccatcctgggacgtgcggctgtggcccctgtcacctggtctgtcatctccctgctgcgtggtgaggcttatgtctgtgctctcagtgagttcgtggacccttcctcactcacggccagggaagagcacttcccatcagcccacgccactgaaatcctggccaggttcccctgcaaggagaaccctgacaacctgtcagacttccgggaggaggtcagccgcaggctcaggtatgagtcccagctctttggatggctgctcatcggcgtggtggccatcctggtgttcctgaccaagtgcctcaagcattactgctcaccactcagctaccgccaggaggcctactgggcgcagtaccgcgccaatgaggaccagctgttccagcgcacggccgaggtgcactctcgggtgctcgctgccaacaatgtgcgccgcttctttggctttgtggcgctcaacaaggatgatgaggaactgattgccaacttcccagtggaaggcacgcagccacggccacagtggaatgccatcaccggcgtctacttgtaccgtgagaaccagggcctcccactctacagccgcctgcacaagtgggcccagggtctggcaggcaacggcgcggcccctgacaacgtggagatggccctgctcccctcctaaggaggtgcttcccatgctctttgtaaatggcactgcttggtcccaaactgaaccccactgcttgctcacatccatatcagaaggggatttttaaaaaactgttatcttcttggccaggggaaaggaccacaagrcaatctggggtgtggacagacccagtagacartggaagccccagccagcagggccaggtgacagtgaagctcaccagtgggctcctttatggtactctatgcagttaacatgtatctagctgcatagggacacccagcgcagcagtgcaccactgggaagtggcctccagtgcagcctctggccttattttatatatttaaatttttgataaagtttttcttactmmaaggaaaaaaaaaaaaaaaaaaaaaaaaa

SEQ ID NO: 14 - protein, 323 aa, Homo sapiens
  UNSURE (39)
MAALIAENFRFLSLFFKSKDVMIFNGLVALGTVGSQELXSVVAFHCPCSPARNYLYGLAAIGVPALVLFIIGIILNNHTWNLVAECQHRRTKNCSAAPTFLLLSSILGRAAVAPVTWSVISLLRGEAYVCALSEFVDPSSLTAREEHFPSAHATEILARFPCKENPDNLSDFREEVSRRLRYESQLFGWLLIGVVAILVFLTKCLKHYCSPLSYRQEAYWAQYRANEDQLFQRTAEVHSRVLAANNVRRFFGFVALNKDDEELIANFPVEGTQPRPQWNAITGVYLYRENQGLPLYSRLHKWAQGLAGNGAAPDNVEMALLPS

SEQ ID NO: 15 - DNA, 1522 bp, Homo sapiens
cgccgacggcggccgagacggacatgaagcaatatcaaggctccggcggcgtcgccatggatgtggaacggagtcgcttcccctactgcgtggtgtggacgcccatcccggtgctcacgtggtttttccccatcatcggccacatgggcatctgcacatccacaggagtcattcgggacttcgcgggcccctactttgtctcagaggacaacatggcctttggaaagcctgccaagtactggaagttggaccctgctcaggtctatgctagcgggcccaacgcatgggacacggctgtgcacgacgcctctgaggagtacaagcaccgcatgcacaatctctgctgtgacaactgccactcgcacgtggcatyggccctgaatctgatgcgctacaacaacagcaccaactggaatatggtgacgctctgcttcttctgcctgctctacgggaagtacgtcagcgttggggccttcgtgaagacctggctgcccttcatccttctcctgggcatcatcctcaccgtcagcctggtctttaacctccggtgatggctgctcggtggccccacacccaccagggtcccgaggaaacagccgccatcccttttggttccagatttttttctcctcaccccaaaaggcagggttgggcctgctgttgtggaccgggggtcggggctggcaggatggaaggactgaggaccagcatgaagtgggggtttgttgtctccctgcctctcagaagcaccctgtcccctcctccccaggcctgtgactccggccctggaagcccctttgttcttctgttgaaaggctttggcttccctctgtagagctgctcccgccaccacctgctggggtcctgcctcagcccagtgcccagtatggggagaggaggacatttgggctcacctgtcaaggtggccctgggaccagagctggtcccarcatggggtgcaccgggtacacttaacgtgtctctataarccaagttgcttcaggaccttcaccactggcctctagaatggtccagaggggctggctgggtccctttgtmagactcctgccggcagctkccctgggggacatgtgtgcccatctggcatcctccagcccgtgcagtccgctcttcactgttccacggcctcccagtgcctcccagcattggacccatctccccctgcagtttgaggccagagaggtgagtggacctgacaagtgccagagtaaccgtgtagacagagcagtgtagacagcgctcagccccagccccaggtgtggacctcatgctggtgatggctcccctgggtggcctgccagcacagccagtkccatcagggagctgaaggggctgtcccccacctaactccagctcccccttcacgttgtcaccaaggccctgtgccgcccgcctcgcccccctgctctgtggattcctttgggaagggctccctgggcaggacaataaagagttttgactccaaaaaaaaaaaaaaaaaaaaaaaaaaaaaaaaaaaaaa

SEQ ID NO: 16 - protein, 112 aa, Homo sapiens
  UNSURE (54)
MAFGKPAKYWKLDPAQVYASGPNAWDTAVHDASEEYKHRMHNLCCDNCHSHVAXALNLMRYNNSTNWNMVTLCFFCLLYGKYVSVGAFVKTWLPFILLLGIILTVSLVFNLR

SEQ ID NO: 17 - DNA, 631 bp, Homo sapiens
cctgttcgcggctctcggcttccactgcagccatgtcactcctcttgctggtggtctcagcccttcacatcctcattcttatactgcttttcgtggccactttggacaagtcctggtggactctccctgggaaagagtccctgaatctctggtacgactgcacgtggaacaacgacaccaaaacatgggcctgcagtaatgtcagcgagaatggctggctgaaggcggtgcaggtcctcatggtgctctccctcattctctgctgtctctccttcatcctgttcatgttccagctctacaccatgcgacgaggaggtctcttctatgccaccggcctctgccagctttgcaccagcgtggcggtgtttactggcgccttgatctatgccattcacgccgaggagatcctggagaagcacccgcgagggggcagcttcggatactgcttcgccctggcctgggtggccttccccctcgccctggtcagcggcatcatctacatccacctacggaagcgggagtgagcgccccgcctcgctcggctgcccccgccccttcccggcccccctcgccgcgcgtcctccaaaaaataaaaccttaaccgcgaaaaaaaaaaaaaaaaaaaaaaaaa

SEQ ID NO: 18 - protein, 163 aa, Homo sapiens
MSLLLLVVSALHILILILLFVATLDKSWWTLPGKESLNLWYDCTWNNDTKTWACSNVSENGWLKAVQVLMVLSLILCCLSFILFMFQLYTMRRGGLFYATGLCQLCTSVAVFTGALIYAIHAEEILEKHPRGGSFGYCFALAWVAFPLALVSGIIYIHLRKRE

SEQ ID NO: 19 - DNA, 737 bp, Homo sapiens
gaaacgtgaagaakgtgaagatggcggtggccagggccggggtcttgggagtccagtggctgcaaagggcatcccggaacgtgatgccgctgggcgcacggacagcctcccacatgaccaaggacatgttcccggggccctatcctaggaccccagaagaacgggccgccgccgccaaraagtataatatgcgtgtggaagactacgaaccttacccggatgatggcatggggtatggcgactacccgaagctccctgaccgctcacagcatgagagagatccatgggatagctgggaccarccgggcctgaggttgaactggggtgaaccgatgcactggcacctagacatgtacaacaggaaccgtgtggatacatcccccacacctgtttcttggcatgtcatgtgtatgcagctcttcggtttcctggctttcatgatattcatgtgctgggtgggggacgtgtaccctgtctaccagcctgtgggaccaaagcagtatccttacaataatytgtacctggrasgrggcggtgatccctccmargraccagrgcgggtggttcactatgagatytgrggrggcttygtgggcttttgggtcctytaamtaggactccctcattcctagaaatttaaccttaatgaaatccctaataaaactcaaaaaaaaaaaaaaaaaaaaaaaaaaaaaaaaaaaaaaaaaaaaaaaaaaaaaaaaaaaaaaaaaaaaaaa

SEQ ID NO: 20 - protein, 196 aa, Homo sapiens
  UNSURE (169)..(170)
  UNSURE (176)..(177)
  UNSURE (179)
  UNSURE (187)
MAVARAGVLGVQWLQRASRNVMPLGARTASHMTKDMFPGPYPRTPEERAAAAKKYNMRVEDYEPYPDDGMGYGDYPKLPDRSQHERDPWDSWDQPGLRLNWGEPMHWHLDMYNRNRVDTSPTPVSWHVMCMQLFGFLAFMIFMCWVGDVYPVYQPVGPKQYPYNNLYLXXGGDPSXXPXRVVHYEIXGGFVGFWVL

SEQ ID NO: 21 - DNA, 1560 bp, Homo sapiens
gtgatattatagtgtagttttttagatgttaccattgggagaaactgagtaaagaagatgtgggatctttctttagtatttcttagaactgcatgtgaatctacaactagctcaaaataaaaagtttaattataaaataaaagctacatgaaatgaagcaaaaaataattcacccttgtcacgcacacagagtcagagactgtaacataatttgcaggatctagagcagaatacaaatgtaaaacatcttgttaaaaaattattaataattttgagacattgataaagcattaagccgcctgtggggccctttaagcatgataaactgtgctaccacacagattgcacattcacgtatctggccctgcaaatggaatgatttttgcccatgatcaattcaccatggcctctttgggctcagtgaatttgcttcttcaggagggtaattttctcttctttctctgctaagctgtttaacagtagttgccctgcctaatgggcttcatccatccatttctctcagattattttcatgatgcactaggatgaagcacaccctttctcctagtcttgaggaaacgtcgatattcagaatatttaaacgcaggcactgaccaatcagaagagtttctggccaacgttccacacttgagggaaatgacattatctgagccctgaagaaaaacgttgtagatattctccagatcaaagcatcgacaggaagattttagatgttgaagttcgtaatatttcctaaagcaggttgtgtgcattcaaaagagcattctattaaagctaccttaatttggcgcttatttttcttaatcatgtttctgacaatcatagtgtgtggaatggttgctgctttaagtgcaataagagctaactgccatcaagagccatcagtatgtcttcaagctgcatgcccagaaagctggattggttttcaaagaaagtgtttctatttttctgatgacaccaagaactggacatcaagtcagaggttttgtgactcacaagatgctgatcttgctcaggttgaaagcttccaggaactgaatttcctgttgagatataaaggcccatctgatcactggattgggctgagcagagaacaaggccaaccatggaaatggataaatggtactgaatggacaagacagtttcctatcctgggagcaggagagtgtgcctatttgaatgacaaaggtgccagtagtgccaggcactacacagagaggaagtggatttgttccaaatcagatatacatgtctagatgttacagcaaagccccaactaatctttagaagcatattggaactgataactccattttaaaatgagcaaagaatttatttcttataccaacaggtatatgaaaatatgctcaatatcactaataactgggaaaatacaaatcaaaatcatagtaaaatattacctgttttcatggtgctaatattacctgttctcccactgctaatgacatacccgagactgagtaatttataaataaaagagatttaattgaaaaaaaaaaaaaaaaaaaaaaaaaaaaa

SEQ ID NO: 22 - protein, 181 aa, Homo sapiens
MLKFVIFPKAGCVHSKEHSIKATLIWRLFFLIMFLTIIVCGMVAALSAIRANCHQEPSVCLQAACPESWIGFQRKCFYFSDDTKNWTSSQRFCDSQDADLAQVESFQELNFLLRYKGPSDHWIGLSREQGQPWKWINGTEWTRQFPILGAGECAYLNDKGASSARHYTERKWICSKSDIHV

SEQ ID NO: 23 - DNA, 3120 bp, Homo sapiens
gtcctacaggctttccactgtggtgtctccagtcattcagagctcatcccctgaaggcctcatcccaatggacactgattcagagtcagcaagccgcttacagatgattctgaatgagattcagcctcgagatactaatgattattttaaccaagccaaaatattgaaagaacatgatagctttgatatgaaggacttgaatgctagtgtggtgaatattgatacttctacagaaatcaaaggtaaagaagtaaaaacatgtgatgtaactgcgcagatggtgctggtatgttgttggagaagtatgaaggaagttgctttacttttaggcatgttgtgccagcttctgcccatgcagcctgtgccagaatcttctgatggattattgacggtggagcaggtaaaagaaataggagattactttaaacaacaccttttgcagtccaggcacagaggagcatttgaattggcttatactggttttgtgaaactcactgaagtactaaacaggtgcccaaatgtgagtctgcaaaagctgccagaacagtggctatggagtgttttagaggaaattaaatgcagtgatccttcatctaaactctgtgctacaaggcgcagtgctggaattcctttctacatacaggcactgttggcatctgaaccaaagaaaggcagaatggatttgttgaaaatagcaatgaaagagttaatctctttggctgggcctacagatgacatacagagtacagtcccccaggttcatgctttaaatatccttagagcattgttcagagatacgcgcctgggagaaaatattattccttatgttgctgatggagctaaggctgcaattctgggttttacatcaccggtctgggcagtgcgaaattcatccacacttctctttagtgccttgatcacaagaatttttggagttaaaagggcaaaggatgaacattccaaaacaaatagaatgacagggagagagtttttctctcgtttcccagaactctatccttttcttctcaaacagttggaaactgtagccaatacagtagacagtgatatgggagaaccaaatcgtcatccaagcatgtttctcttacttttggtgttggagagactctacgcttccccgatggatggtacttcttctgctctcagcatgggaccttttgttcccttcattatgaggtgtggtcactcacctgtctaccactcccgtgaaatggcagctcgtgccttggtcccatttgttatgatagatcacattcctaataccattcgaactctgttgtccacactccccagctgcactgaccagtgtttccggcaaaaccacattcatgggacacttctccaggtttttcatttgttgcaagcctactcagactccaaacacggaacgaattcagacttccagcacgagctgactgacatcactgtttgtaccaaagccaaactctggctggccaagaggcaaaatccatgtttggtgaccagagctgtatatattgatattctcttcctattgacttgctgcctcaacagatctgcaaaggacaaccagccagttctggagagtcttggcttctgggaggaagtcagagggattatctcaggatcagagctgataacgggattcccttgggccttcaaggtgccaggcctgccccagtacctccagagcctcaccagactagccattgctgcagtgtgggccgcggcagccaagagtggagagcgggagacgaatgtccccatctctttctctcagctgttagaatctgccttccctgaagtgcgctcactaacactggaagccctcttggaaaagttcttagcagcagcctctggacttggagagaagggcgtgccacccttgctgtgcaacatgggagagaagttcttattgttggccatgaaggaaaatcacccagaatgcttctacaagatactgaaaattctccactgcatggaccctggtgagtggcttccccagacggagcactgtgtccatctgaccccaaaggagttcttgatctggacgatggatattgcttccaatgaaagatctgaaattcagagtgtagctctgagacttgcttccaaagtcatttcccaccacatgcagacatgtgtggagaacagggaattgatagctgctgagctgaagcagtgggttcagctggtcatcttgtcatgtgaagaccatcttcctacagagtctaggctggccgtcgttgaagtcctcaccagtactacaccacttttcctcaccaacccccatcctattcttgagttgcaggatacacttgctctctggaagtgtgtccttacccttctgcagagtgaggagcaagctgttagagatgcagccacggaaaccgtgacaactgccatgtcacaagaaaatacctgccagtcaacagagtttgccttctgccaggtggatgcctccatcgctctggccctggccctggccgtcctgtgtgatctgctccagcagtgggaccagttggcccctggactgcccatcctgctgggatggctgttgggagagagtgatgacctcgtggcctgtgtggagagcatgcatcaggtggaagaagactacctgtttgaaaaagcagaagtcaacttttgggccgagaccctgatctttgtgaaatacctctgcaagcacctcttctgtctcctctcaaagtccggctggcgtcccccaagccctgagatgctctgtcaccttcaaaggatggtgtcagagcagtgccacctcctgtctcagttcttcagagagcttccaccagctgctgagtttgtgaagacagtggagttcacaagactacgcattcaagaggaaaggactttggcttgcttgaggctgctggcctttttggaaggaaaggaaggggaagacaccctagttctcagtgtttgggactcttatgcagaatcgaggcagttaactcttccaagaacagaagcggcatgttgaagaaaatctgggggattgggatgggggtatgtgtggatttttcctccactaaatctgcaggaaacatgttgaacataaattcaaaaattttatcccaaaaaaaaaaaaaaaaaaaaaaaaaa

SEQ ID NO: 24 - protein, 976 aa, Homo sapiens
MDTDSESASRLQMILNEIQPRDTNDYFNQAKILKEHDSFDMKDLNASVVNIDTSTEIKGKEVKTCDVTAQMVLVCCWRSMKEVALLLGMLCQLLPMQPVPESSDGLLTVEQVKEIGDYFKQHLLQSRHRGAFELAYTGFVKLTEVLNRCPNVSLQKLPEQWLWSVLEEIKCSDPSSKLCATRRSAGIPFYIQALLASEPKKGRMDLLKIAMKELISLAGPTDDIQSTVPQVHALNILRALFRDTRLGENIIPYVADGAKAAILGFTSPVWAVRNSSTLLFSALITRIFGVKRAKDEHSKTNRMTGREFFSRFPELYPFLLKQLETVANTVDSDMGEPNRHPSMFLLLLVLERLYASPMDGTSSALSMGPFVPFIMRCGHSPVYHSREMAARALVPFVMIDHIPNTIRTLLSTLPSCTDQCFRQNHIHGTLLQVFHLLQAYSDSKHGTNSDFQHELTDITVCTKAKLWLAKRQNPCLVTRAVYIDILFLLTCCLNRSAKDNQPVLESLGFWEEVRGIISGSELITGFPWAFKVPGLPQYLQSLTRLAIAAVWAAAAKSGERETNVPISFSQLLESAFPEVRSLTLEALLEKFLAAASGLGEKGVPPLLCNMGEKFLLLAMKENHPECFYKILKILHCMDPGEWLPQTEHCVHLTPKEFLIWTMDIASNERSEIQSVALRLASKVISHHMQTCVENRELIAAELKQWVQLVILSCEDHLPTESRLAVVEVLTSTTPLFLTNPHPILELQDTLALWKCVLTLLQSEEQAVRDAATETVTTAMSQENTCQSTEFAFCQVDASIALALALAVLCDLLQQWDQLAPGLPILLGWLLGESDDLVACVESMHQVEEDYLFEKAEVNFWAETLIFVKYLCKHLFCLLSKSGWRPPSPEMLCHLQRMVSEQCHLLSQFFRELPPAAEFVKTVEFTRLRIQEERTLACLRLLAFLEGKEGEDTLVLSVWDSYAESRQLTLPRTEAAC

SEQ ID NO: 25 - DNA, 1078 bp, Homo sapiens
cttcatcacaggatcaacacatttcatctgggcttcttaaatctaaatctttaaaatgactaagttttcttccttttctctgtttttcctaatagttggggcttatatgactcatgtgtgtttcaatatggaaattattggagggaaagaagtgtcacctcattccaggccatttatggcctccatccagtatggcggacatcacgtttgtggaggtgttctgattgatccacagtgggtgctgacagcagcccactgccaatatcggtttaccaaaggccagtctcccactgtggttttaggcgcacactctctctcaaagaatgaggcctccaaacaaacactggagatcaaaaaatttataccattctcaagagttacatcagatcctcaatcaaatgatatcatgctggttaagcttcaaacagccgcaaaactcaataaacatgtcaagatgctccacataagatccaaaacctctcttagatctggaaccaaatgcaaggttactggctggggagccaccgatccagattcattaagaccttctgacaccctgcgagaagtcactgttactgtcctaagtcgaaaactttgcaacagccaaagttactacaacggcgacccttttatcaccaaagacatggtctgtgcaggagatgccaaaggccagaaggattcctgtaagggtgactcagggggccccttgatctgtaaaggtgtcttccacgctatagtctctggaggtcatgaatgtggtgttgccacaaagcctggaatctacaccctgttaaccaagaaataccagacttggatcaaaagcaaccttgttccgcctcatacaaattaagttacaaataattttattggatgcacttgcttcttttttcctaatatgctcgcaggttagagttgggtgtaagtaaagcagagcacatatggggtccatttttgcacttgtaagtcattttattaaggaatcaagttctttttcacttgtatcactgatgtatttctaccatgctggttttattctaaataaaatttagaagactctcaaaaaaaaaaaaaaaaaaaa

SEQ ID NO: 26 - protein, 264 aa, Homo sapiens
MTKFSSFSLFFLIVGAYMTHVCFNMEIIGGKEVSPHSRPFMASIQYGGHHVCGGVLIDPQWVLTAAHCQYRFTKGQSPTVVLGAHSLSKNEASKQTLEIKKFIPFSRVTSDPQSNDIMLVKLQTAAKLNKHVKMLHIRSKTSLRSGTKCKVTGWGATDPDSLRPSDTLREVTVTVLSRKLCNSQSYYNGDPFITKDMVCAGDAKGQKDSCKGDSGGPLICKGVFHAIVSGGHECGVATKPGIYTLLTKKYQTWIKSNLVPPHTN

SEQ ID NO: 27 - DNA, 2238 bp, Homo sapiens
ggcaaaatgcatgacagtaacaatgtggagaaagacattacaccatctgaattgcctgcaaagccaggttgtctgcattcaaaagagcattctattaaagctaccttaatttggcgcttatttttcttaatcatgtttctgacaatcatagtgtgtggaatggttgctgctttaagtgcaataagagctaactgccatcaagagccatcagtatgtcttcaagctgcatgcccagaaagctggattggttttcaaagaaagtgtttctatttttctgatgacaccaagaactggacatcaagtcagaggttttgtgactcacaagatgctgatcttgctcaggttgaaagcttccaggaactgaatttcctgttgagatataaaggcccatctgatcactggattgggctgagcagagaacaaggccaaccatggaaatggataaatggtactgaatggacaagacagttagtcatgaaagaagatggtgccaacttgtatgttgcaaaggtttcacaagttcctcgaatgaatccaagacctgtcatggtttcctatcctgggagcaggagagtgtgcctatttgaatgacaaaggtgccagtagtgccaggcactacacagagaggaagtggatttgttccaaatcagatatacatgtctagatgttacagcaaagccccaactaatctttagaagcatattggaactgataactccattttaaaatgagcaaagaatttatttcttataccaacaggtatatgaaaatatgctcaatatcactaataactgggaaaatacaaatcaaaatcatagtaaaatattacctgttttcatggtgctaatattacctgttctcccactgctaatgacatacccgagactgagtaatttataaataaaagagatttaattgactcatagttccacatggctggggaggtcttgcaatcatgacagaaggcaaatgggaagcaaagtcatgtcttatgtggtggcgggcagggggacttgtgcacaggaactcctatttatacaaccatcagatatcttgagacaagaacagtatggggctccctggtgtgattccgtcctgcgcggctgttctctggagcagcattcatttatcttcgtctgccttgtctcctacctaagtgtgtgtcgccacccgatggaagatttgatggacatggacatgagccccctgaggccccagaactatcttttcagttgtgaactaaaggccgacaaagatgatcactttaaggtggataatgatgaaaatgagaccagttatctttaagaacggtcagctcaggggctggtgcaaaggatgaactgcacattgttgaagcagaggccatgaatgacgaaggcagtccaattaaagtaacactggcaactttgaaaatgtctgtacagccaacggtttctcttgggggctttgaaataacaccaccagtggacttaaggttgaagtgtggttcagggccagtgcatattagtggacagcacttagtagctgtgaaggaaggtgcagagtcagaagatgaagaagaggaggatgtgaaactcttaagtatatctggaaagcagtctgcccctggaggtgggcagaaaaaagtaaaacttgctgctgctgctgctgatgatgatgatgaagatgatgatgatgatgatgacgaggaagctgaagaaaaagcgccagtgaagaaatctatacgagatactccagccaaaaatgcacaaaagtcaaatcagaatggaaaagactcaaaaccatcatcaacaccaagatcaaaaggacaagaatccttcaaaaaacaggaaaaatctcctaaaacaccaaaaggatctagttctgtagaagacattaaagcaaaaatgcaagcaagtatagaaaaacgtggttctcttcccaaagtggaaaccaagttcatcaattatgtgaagaatttcttctggatgactgaccaagaggctattcaagatctctggcagtggaggaagtctctttaagaaaatagtttaaacaatttgttaaaaattttccatcttatttcatttctgtaacagttgatatctggctgtcctttttatagtgcagagtgagaactttccctaccatgtttgataaatgttgtccaggttctattgcccaaaaaaaaaaaaaaaaaaaaaaaaaaaaaaaaaaaaaaaaaaaaaaaaaaaaaaaaaaaaaaaaaaaaaaaaaaaa

SEQ ID NO: 28 - protein, 194 aa, Homo sapiens
MHDSNNVEKDITPSELPAKPGCLHSKEHSIKATLIWRLFFLIMFLTIIVCGMVAALSAIRANCHQEPSVCLQAACPESWIGFQRKCFYFSDDTKNWTSSQRFCDSQDADLAQVESFQELNFLLRYKGPSDHWIGLSREQGQPWKWINGTEWTRQLVMKEDGANLYVAKVSQVPRMNPRPVMVSYPGSRRVCLFE

SEQ ID NO: 29 - DNA, 1024 bp, Homo sapiens
ggactgcagctgtggggagatttcagtgcattgcctcccctgggtgctcttcatcttggatttgaaagttgagagcagcatgttttgcccactgaaactcatcctgctgccagtgttactggattattccttgggcctgaatgacttgaatgtttccccgcctgagctaacagtccatgtgggtgattcagctctgatgggatgtgttttccagagcacagaagacaaatgtatattcaagatagactggactctgtcaccaggagagcacgccaaggacgaatatgtgctatactattactccaatctcagtgtgcctattgggcgcttccagaaccgcgtacacttgatgggggacatcttatgcaatgatggctctctcctgctccaagatgtgcaagaggctgaccagggaacctatatctgtgaaatccgcctcaaaggggagagccaggtgttcaagaaggcggtggtactgcatgtgcttccagaggagcccaaagagctcatggtccatgtgggtggattgattcagatgggatgtgttttccagagcacagaagtgaaacacgtgaccaaggtagaatggatattttcaggacggcgcgcaaaggtaacaaggaggaaacatcactgtgttagagaaggctctggctgatggtatcaggacaaaggtagaatcaggcacatgaggaggtgttgcaagagcctgggctttggtgcttatcagaactggaccttctcctagcaatttcagctttctggtgggaaagataactccaatgaagaacaagaacaagaagatgatgatgatgcttaactttttggatgccgatatgagattgtacatgtaaagcattttgtataagacttggcccctgcattttagtttccttctttctcccttttccttcgtatagagtccatgggagaatgagggagatgatttttgtggcccagccaagaaagcaatgggctagacattaaagtgattacacttttattcttaaaaaaaaaaaaaaaa

SEQ ID NO: 30 - protein, 192 aa, Homo sapiens [sequence omitted]

SEQ ID NO: 31 - DNA, 701 bp, Homo sapiens
ctagtttggagctgtgctgtaaaaacaagagtaacatttttatattaaagttaaataaagttacaactttgaagagagtttctgcaagacatgacacaaagctgctagcagaaaatcaaaacgctgattaaaagaagcacggtatgatgaccaaacataaaaagtgttttataattgttggtgttttaataacaactaatattattactctgatagttaaactaactcgagattctcagagtttatgcccctatgattggattggtttccaaaacaaatgctattatttctctaaagaagaaggagattggaattcaagtaaatacaactgttccactcaacatgccgacctaactataattgacaacatagaagaaatgaattttcttaggcggtataaatgcagttctgatcactggattggactgaagatggcaaaaaatcgaacaggacaatgggtagatggagctacatttaccaaatcgtttggcatgagagggagtgaaggatgtgcctacctcagcgatgatggtgcagcaacagctagatgttacaccgaaagaaaatggatttgcaggaaaagaatacactaagttaatgtctaagataatggggaaaatagaaaataacattattaagtgtaaaaccagcaaagtacttttttaattaaacaaagttcgagtttaaaaaaaaaaaaaaaa

SEQ ID NO: 32 - protein, 149 aa, Homo sapiens
MMTKHKKCFIIVGVLITTNIITLIVKLTRDSQSLCPYDWIGFQNKCYYFSKEEGDWNSSKYNCSTQHADLTIIDNIEEMNFLRRYKCSSDHWIGLKMAKNRTGQWVDGATFTKSFGMRGSEGCAYLSDDGAATARCYTERKWICRKRIH

SEQ ID NO: 33 - DNA, 863 bp, Homo sapiens
ggaacttcgttatccgcgatgcgtttcctggcagctacattcctgctcctggcgctcagcaccgctgcccaggccgaaccggtgcagttcaaggactgcggttctgtggatggagttataaaggaagtgaatgtgagcccatgccccacccaaccctgccagctgagcaaaggacagtcttacagcgtcaatgtcaccttcaccagcaatattcagtctaaaagcagcaaggccgtggtgcatggcatcctgatgggcgtcccagttccctttcccattcctgagcctgatggttgtaagagtggaattaactgccctatccaaaaagacaagacctatagctacctgaataaactaccagtgaaaagcgaatatccctctataaaactggtggtggagtggcaacttcaggatgacaaaaaccaaagtctcttctgctgggaaatcccagtacagatcgtttctcatctctaagtgcctcattgagttcggtgcatctggccaatgagtctgctgagactcttgacagcacctccagctctgctgcttcaacaacagtgacttgctctccaatggtatccagtgattcgttgaagaggaggtgctctgtagcagaaactgagctccgggtggctggttctcagtggttgtctcatgtctctttttctgtcttaggtggtttcattaaatgcagcacttggttagcagatgtttaattttttttttaacaacattaacttgtggcctctttctacacctggaaatttactcttgaataaataaaaactcgtttgtcttgtccaaaaaaaaaaaaaaaaaaaaaaaaaaaaaaaaaaaaaaaaaaaaaaaaaaaaaaaaaaaaa

SEQ ID NO: 34 - protein, 151 aa, Homo sapiens
MRFLAATFLLLALSTAAQAEPVQFKDCGSVDGVIKEVNVSPCPTQPCQLSKGQSYSVNVTFTSNIQSKSSKAVVHGILMGVPVPFPIPEPDGCKSGINCPIQKDKTYSYLNKLPVKSEYPSIKLVVEWQLQDDKNQSLFCWEIPVQIVSHL

SEQ ID NO: 35 - DNA, 1575 bp, Homo sapiens
cataacagtccaggaaaccaacaaaaataactctgaaagcattgaatgcagcaaaataacaatggatctcaagttcaacaattccaggaaatatatttctatcactgtgccatccaaaacccaaacaatgtcaccacacatcaagtcagttgacgacgttgtggtacttggcatgaatctcagcaagtttaacaaacttactcagtttttcatatgtgttgctggagtttttgtattttacctaatttatgggtatttacaggaattaatattttcagtggagggttttaagtcctgtggctggtaccttaccttagtgcagtttgccttttactccatatttggcctaatagaacttcagcttattcaggacaaaaggaggagaataccaggaaaaacctacatgataatagcttttctaactgtgggtactatggggttatcaaacacttccttgggctacctgaattaccctacccaagtcatcttcaagtgctgcaaattgattcctgttatgctaggaggagtttttattcaaggaaagcgttataatgttgcagatgtgtctgctgccatatgtatgagccttggcctgatatggtttaccctcgctgacagcacaactgcaccaaatttcaacctgacgggtattgtattcgtattcaattggttttgtatacattttactgggattgacatgcactagtggattaggccctgcagtaacattttgtgcaaagaatccagttcggacctatggttatgcgttccttttttccctcactggatattttggaatctcctttgttctggctttgattaaaatttttggtgcacttattgctgtaacagtgacaacaggaagaaaagcaatgaccattgtactttcgtttatattctttgctaaaccattcacgtttcagtatgtatggtctggtttgttagttgtccttggtatatttctcaatgtttacagcaaaaatatggataaaataagactaccatcactgtatgatttgataaacaaatcagtggaagcaagaaagtcaaggacgctggcacagactgtatagacagtgattgtcctatttaaaatagaattttaagggaacaatcatcaattaattaactttccaaagggactgataaaaaccaaaggatctggaggcattgctatcccatttgtggacagatttcatatgaagttgttttgcggtgtcagccttttcttcagagcatttgtttgactgacttccaaagcaatcaagagagccacgtctagcagactttacaataaaatgtcaatatgaaggactgtaattcctagcagtttattgagaatttcactggaaatggaccatgtgttgcaagactaattggctataattatatcctatcaaagaaatcgatacgtaatagcagattgttttatattcattccattttgatggtgttatttaaattgattctctgttataagagtaaactgatgagttgaagtctggagagaataacattcttataaataaaattattctgtgatcttttttccaaaaaaaaaaaaaaaaaaaaaaa

SEQ ID NO: 36 - protein, 214 aa, Homo sapiens
MDLKFNNSRKYISITVPSKTQTMSPHIKSVDDVVVLGMNLSKFNKLTQFFICVAGVFVFYLIYGYLQELIFSVEGFKSCGWYLTLVQFAFYSIFGLIELQLIQDKRRRIPGKTYMIIAFLTVGTMGLSNTSLGYLNYPTQVIFKCCKLIPVMLGGVFIQGKRYNVADVSAAICMSLGLIWFTLADSTTAPNFNLTGIVFVFNWFCIHFTGIDMH

SEQ ID NO: 37 - DNA, 1686 bp, Homo sapiens
cgataatcttcttccatttttgcggaaatttattgcatccttctttaaaccggggtttgagaagtataataacttggatctgtttcggtatctcttaaatattccaggaccaattgacattccatctcgtttatgtaaagggaattttgatgatgatatgtttaaccaccaagttccttatttgtggctgatttactgcctttgtcatcctcttcaatcaagtattaaagaaacagtggaggcatatgaggcagcattaggggtggctatgagatgtgatatagtacagaagatatggatggattatcttgtctttgcaaataatagagctgctggatccagaaacaaagttcaagaattcaaattttttactgatttagtgaatagatgtttggttacagtccctgcccgataccccattccttttagcagtgctgattactggtccaactatgaatttcataatagggttattttcttttatttgagctgtgttccaaagacccagcattccaaaaccttggaacggttttgttcagttatgccagctaattctggacttgcattgaggttacttcaacatgaatgggaagaaagcaatgttcagattctgaaacttcaagccaagatgtttacatataatatcccaacatgcctggccacctggaaaatagccattgctgctgagattgttctaaagggacaaagagaggtccaccgtttatatcagagagccttacagaagttacctctttgtgcatcactgtggaaagatcaactcttgtttgaagcatcagaaggaggtaaaactgataacctgagaaaactagtttccaagtgccaagagattggagtcagcctaaatgagctcttaaatttaaacagtaacaaaacagaaagcaagaatcactgaacactgggtgcagtcagttctaagtccttataataattgccaaaattatttgaatgattcttcaagattaggctgatccctggctaaggtctgtgtaaggcagacaagcgttattgatcatatcaagttccctacaatatcctgtcctcaaaaccggaagcaatgaacatgatcctcttcggttggataaatgaacttcctgtttggcctgcttctaggccctgccagattctcataacatcatatacgtaagtatagttcctcaaagtgactgacatttattttaattttgctttgtttttttttattttctcccccattcctttattttgtgttattcctgactcacttgacactctctgatgcctgagagattcctgtttgggatttaatatccagggctgtgtttacagtaaaaaaagcaggcagtcccttttagtttttcctttttaaatttttttgagattcttcatttcaggattttaaaactatagcagtccatcttaaggaaagtgtaactgccatggccacaagtctgctagttgcacttgaatgctctatcagggttgtttattaccctttctacgttctggactccttgccgagactgtttaacttgaagattaaagaaactattgcaaatgccagtgcatcagaacctaagagtggtcaaatattatgtgcaatttttttgtaaagaaattttaatttataataaagtttaacagtttaaagaacaaaaaaaaaaaaaaaaaaaaaaaaaaaaaaaaaaa

SEQ ID NO: 38 - protein, 303 aa, Homo sapiens
DNLLPFLRKFIASFFKPGFEKYNNLDLFRYLLNIPGPIDIPSRLCKGNFDDDMFNHQVPYLWLIYCLCHPLQSSIKETVEAYEAALGVAMRCDIVQKIWMDYLVFANNRAAGSRNKVQEFKFFTDLVNRCLVTVPARYPIPFSSADYWSNYEFHNRVIFFYLSCVPKTQHSKTLERFCSVMPANSGLALRLLQHEWEESNVQILKLQAKMFTYNIPTCLATWKIAIAAEIVLKGQREVHRLYQRALQKLPLCASLWKDQLLFEASEGGKTDNLRKLVSKCQEIGVSLNELLNLNSNKTESKNH

SEQ ID NO: 39 - DNA, 2679 bp, Homo sapiens
gcggcggcggctggggcgttcgcgggccggcgcgcggcgtgcggggccgtgctgctgacggagctgctggagcgcgccgctttctacggcatcacgtccaacctggtgctattcctgaacggggcgccgttctgctgggagggcgcgcaggccagcgaggcgctgctgctcttcatgggcctcacctacctgggctcgccgttcggaggctggctggccgacgcgcggctgggccgggcgcgcgccatcctgctgagcctggcgctctacctgctgggcatgctggccttcccgctgctggccgcgcccgccacgcgagccgcgctctgcggttccgcgcgcctgctcaactgcacggcgcctggtcccgacgccgccgcccgctgctgctcaccggccaccttcgcggggctggtgctggtgggcctgggcgtggccaccgtcaaggccaacatcacgcccttcggcgccgaccaggttaaagatcgaggtccggaagccactaggagattttttaattggttttattggagcattaacctgggagcgatcctgtcgttaggtggcattgcctatattcagcagaacgtcagctttgtcactggttatgcgatccccactgtctgcgtcggccttgcttttgtggtcttcctctgtggccagagcgttttcatcaccaagcctcctgatggcagtgccttcaccgacatgttcaagatactgacgtattcctgctgttcccagaagcgaagtggagagcgccagagtaatggtgaaggcattggagtctttcagcaatcttctaaacaaagtctgtttgattcatgtaagatgtctcatggtgggccatttacagaagagaaagtggaagatgtgaaagctctggtcaagattgtccctgttttcttggctttgataccttactggacagtgtatttccaaatgcagacaacatatgttttacagagtcttcatttgaggattccagaaatttcaaatattacaaccactcctcacacgctccctgcagcctggctgaccatgtttgatgctgtgctcatcctcctgctcatccctctgaaggacaaactggtcgatcccattttgagaagacatggcctgctcccatcctccctgaagaggatcgccgtgggcatgttctttgtcatgtgctcagcctttgctgcaggaattttggagagtaaaaggctgaaccttgttaaagagaaaaccattaatcagaccatcggcaacgtcgtctaccatgctgccgatctgtcgctgtggtggcaggtgccgcagtacttgctgattgggatcagcgagatctttgcaagtatcgcaggcctggaatttgcatactcagctgcccccaagtccatgcagagtgccataatgggcttgttctttttcttctctggcgtcgggtcgttcgtgggttctggactgctggcactggtgtctatcaaagccatcggatggatgagcagtcacacagactttggtaatattaacggctgctatttgaactattacttttttcttctggctgctattcaaggagctaccctcctgcttttcctcattatttctgtgaaatatgaccatcatcgagaccatcagcgatcaagagccaatggcgtgcccaccagcaggagggcctgaccttcctgaggccatgtgcggtttctgaggctgacatgtcagtaactgactggggtgcactgagaacaggcaagactttaaattcccataaaatgtctgacttcactgaaacttgcatgttgcctggattgatttcttctttccctctatccaaaggagcttggtaagtgccttactgcagcgtgtctcctggcacgctgggccctccgggaggagagctgcagatttcgagtatgtcgcttgtcattcaaggtctctgtgaatcctctagctgggttcccttttttacagaaactcacaaatggagattgcaaagtcttggggaactccacgtgttagttggcatcccagtttcttaaacaaatagtatcacctgcttcccatagccatatctcactgtaaaaaaaaaaattaataaactgttacttatatttaagaaagtgaggattttttttttttaaagataaaagcatggtcagatgctgcaaggattttacataaatgccatatttatggtttccttcctgagaacagtcttgctcttgccatgttctttgatttaggctggtagtaaacacatttcatctgctgcttcaaaaagtacttactttttaaaccatcaacattacttttctttcttaaggcaaggcatgcataagagtcatttgagaccatgtgtcccatctcaagccacagagcaactcacggggtacttcacaccttacctagtcagagtgcttatatatagctttattttggtacgattgagactaaagactgatcatggttgtatgtaaggaaaacattcttttgaacagaaatagtgtaattaaaaataattgaaagtgttaaatgtgaacttgagctgtttgaccagtcacatttttgtattgttactgtacgtgtatctggggcttctccgtttgttaatactttttctgtatttgttgctgtatttttggcataactttattataaaaagcatctcaaatgcgaaaaaaaaaaaaaaaaaaa

SEQ ID NO: 40 - protein, 554 aa, Homo sapiens
AAAAGAFAGRRAACGAVLLTELLERAAFYGITSNLVLFLNGAPFCWEGAQASEALLLFMGLTYLGSPFGGWLADARLGRARAILLSLALYLLGMLAFPLLAAPATRAALCGSARLLNCTAPGPDAAARCCSPATFAGLVLVGLGVATVKANITPFGADQVKDRGPEATRRFFNWFYWSINLGAILSLGGIAYIQQNVSFVTGYAIPTVCVGLAFVVFLCGQSVFITKPPDGSAFTDMFKILTYSCCSQKRSGERQSNGEGIGVFQQSSKQSLFDSCKMSHGGPFTEEKVEDVKALVKIVPVFLALIPYWTVYFQMQTTYVLQSLHLRIPEISNITTTPHTLPAAWLTMFDAVLILLLIPLKDKLVDPILRRHGLLPSSLKRIAVGMFFVMCSAFAAGILESKRLNLVKEKTINQTIGNVVYHAADLSLWWQVPQYLLIGISEIFASIAGLEFAYSAAPKSMQSAIMGLFFFFSGVGSFVGSGLLALVSIKAIGWMSSHTDFGNINGCYLNYYFFLLAAIQGATLLLFLIISVKYDHHRDHQRSRANGVPTSRRA

SEQ ID NO: 41 - DNA, 549 bp, Homo sapiens
atcaccctcagcagcagcagcagcagccgcaccaccaccaccattattatttctacaaccacagccacaaccaccaccaccaccatcatcaccagcagcctcaccaatacctgcagcatggagccgagggcagccccaaggcccagccaaagccgctgaaacatgagcagaaacacaccctccagcagcaccaggaaacgccgaagaagaaaacaggctatggtgaactaaacggtaatgctggagaaagagaaatatctttaaagaacctgagttctgatgaagccaccaaccctatttccagggtcctcaatggcaaccagcaagttgtagacactagcctgaagcagactgtaaaggccaacacctttgggaaagcaggaattaaaaccaagaatttcattcagaaaaacagtatggacaaaaagaatgggaagtcttatgaaaataaatctggagagaatcacagatgtggaatatttgtcataaataaataatgaaaacctaaaaaaaaaaaaaaaaaaaaaaaaaaaaaaaaaaaaaaaaaaa

SEQ ID NO: 42 - protein, 164 aa, Homo sapiens
HPQQQQQQPHHHHHYYFYNHSHNHHHHHHHQQPHQYLQHGAEGSPKAQPKPLKHEQKHTLQQHQETPKKKTGYGELNGNAGEREISLKNLSSDEATNPISRVLNGNQQVVDTSLKQTVKANTFGKAGIKTKNFIQKNSMDKKNGKSYENKSGENHRCGIFVINK

SEQ ID NO: 43 - DNA, 453 bp, Homo sapiens
agtttactctccttctagctttctgcttaccgcacactggataacacacacatacacacccacaaaaatgctcatgaacccaatccggagaaggttccagcaggtcccccaccctcccctcctcctcctacttctcctcttgacagcgaggacaggagggggacaaggggacacctgggcagacccgccggctctccccccaccccaccccgcccctcacatcatactccaatcataaccttgtatattacgcagtcattttggttttcgcggacgcgcctacctaagtaccatttacagaaagtgactctggctgtcattattttgtttatttgttccctatgcaaaaaaaaaatgaaaatgaaaaaagggggattccataaaagattcaataaaagacaaaaaaaaagaaaaaagaaaaaaatgtataaaaattaaaaaaaaaaaaaaaaa

SEQ ID NO: 44 - protein, 56 aa, Homo sapiens
MLMNPIRRRFQQVPHPPLLLLLLLLTARTGGGQGDTWADPPALPPPHPAPHIILQS

SEQ ID NO: 45 - DNA, 690 bp, Homo sapiens
gagatctggatggcatctacttcgtatgactattgcagagtgcccatggaagacggggataagcgctgtaagcttctgctggggataggaattctggtgctcctgatcatcgtgattctgggggtgcccttgattatcttcaccatcaaggccaacagcgaggcctgccgggacggccttcgggcagtgatggagtgtcgcaatgtcacccatctcctgcaacaagagctgaccgaggcccagaagggctttcaggatgtggaggcccaggccgccacctgcaaccacactgtgatggccctaatggcttccctggatgcagagaaggcccaaggacaaaagaaagtggaggagcttgagggagagatcactacattaaaccataagcttcaggacgcgtctgcagaggtggagcgactgagaagagaaaaccaggtcttaagcgtgagaatcgcggacaagaagtactaccccagctcccaggactccagctccgctgcggcgccccagctgctgattgtgctgctgggcctcagcgctctgctgcagtgagatcccaggaagctggcacatcttggaaggtccgtcctgctcggcttttcgcttgaacattcccttgatctcatcagttctgagcgggtcatggggcaacacggttagcgggacgagactcgactaatacgtacataa

SEQ ID NO: 46 - protein, 180 aa, Homo sapiens
MASTSYDYCRVPMEDGDKRCKLLLGIGILVLLIIVILGVPLIIFTIKANSEACRDGLRAVMECRNVTHLLQQELTEAQKGFQDVEAQAATCNHTVMALMASLDAEKAQGQKKVEELEGEITTLNHKLQDASAEVERLRRENQVLSVRIADKKYYPSSQDSSSAAAPQLLIVLLGLSALLQ

SEQ ID NO: 47 - DNA, 2350 bp, Homo sapiens
tgactgcttcactctctcattcttagcttgaatttggaaatgacttttgatgacctaaagatccagactgtgaaggaccagcctgatgagaagtcaaatggaaaaaaagctaaaggtcttcagtttctttactctccatggtggtgcctggctgctgcgactctaggggtcctttgcctgggattagtagtgaccattatggtgctgggcatgcaattatcccaggtgtctgacctcctaacacaagagcaagcaaacctaactcaccagaaaaagaaactggagggacagatctcagcccggcaacaagcagaagaagcttcacaggagtcagaaaacgaactcaaggaaatgatagaaacccttgctcggaagctgaatgagaaatccaaagagcaaatggaacttcaccaccagaatctgaatctccaagaaacactgaagagagtagcaaattgttcagctccttgtccgcaagactggatctggcatggagaaaactgttacctattttcctcgggctcatttaactgggaaaagagccaagagaagtgcttgtctttggatgccaagttgctgaaaattaatagcacagctgatctgatttagagtccgaggcgctgtctcccagacatacccttcaggtacctgtgcatatatacaacgaggagctgtttatgcggaaaactgcattttagctgccttcagtatatgtcagaagaaggcaaacctaagagcacagtgaatttgaaggctctggaagaaaagaaaaaagtctttgagttttattctggaatttaagctattctttgtcacttgggtgccaaacatgagagcccagaaaactgtcatttagctggctgcagaactcctttgcagaaactggggttccaggtgcctggcacctttatgtcaacatttttgattctagctacctgtattatttcacctagcttgtcccaagcttccctgccagcctgaagtccattttcccctttttattttaaaatttgactcctcttcaagcttgaaaaccctctgaactcagtcttctttacctcattatcaccttcccctcacactcctaaaattgcatgaaagacagaacatggagaacttgctcaagtgcaggcagagagcaaaaaggggaaatatgtctgggaaaaagtgcacgtgaagaaacaaagaaggacagaggccattccgaaatcaagaaactcatgttcttaactttaaaaaaggtatcaatccttggtttttaaactgtggtccatctccagactctaccacttacggacagacagacagacacacacacacacacacacacacacacacacacattttgggacaagtggggagcccaagaaagtaattagtaagtgagtggtcttttctgtaagctaatccacaacctgttaccacttcctgaatcagttattatttcttcatttttttttctaccagaggacagattaatagatttaacccttcacaacagttcttgttagaatcatgggatgtgtggcccagaggtaagaatagaatttctttccctaaagaacataccttttgtagatgaactcttctcaactctgttttgctatgctataattccgaaacatacaagacaaaaaaaatgaagacactcaatctagaacaaactaagccaggtatgcaaatatcgctgaatagaaacagatggaattagaaatatatcttctatttttaggcttctatttcctttccacccactcttcacaggctattctactttaaaggaagcctttttattttgctgcacacaatctagcaggaatctttttttttttttaagagctgtgtcatccttatgtaggcaagagatgtttgcttttgttaaaagctttattgagatataattaacataaaataaactgaacatatttaaagtgtactatttgataagttttcacaccttgtggagaacatgcatactacaattaagagagtgaacatatccatcatccctcaaagtgtcacaatgctcctcctgatgactcctccccagaaaaccaccaatcggctttcattttgcattttgtagttttatgtgaatggaatcatatagtatgtcttttttttttgtctggcttctttcactttgcataattattttgagattcatatgtctccatcttgatgctcgtatgaattcattcttttaaatgttgaatattcccttgtatggatataccacaattcatttacccatttacttgttgatgacatttgggttgttttagttttgggatattacaaataaagctgctgtgaacatttgtgtacaaaaaaaaaaaaaaaaaaaaaa

SEQ ID NO: 48 - protein, 189 aa, Homo sapiens
MTFDDLKIQTVKDQPDEKSNGKKAKGLQFLYSPWWCLAAATLGVLCLGLVVTIMVLGMQLSQVSDLLTQEQANLTHQKKKLEGQISARQQAEEASQESENELKEMIETLARKLNEKSKEQMELHHQNLNLQETLKRVANCSAPCPQDWIWHGENCYLFSSGSFNWEKSQEKCLSLDAKLLKINSTADLI

SEQ ID NO: 49 - DNA, 438 bp, Homo sapiens
cggggaccaagtggcaacgacttggacatctgagctgtcactgccgaaaacaggccgcaagagagataatcaatatgcatttccaagccttttggctatgtttgggtcttctgttcatctcaattaatgcagaatttatggatgatgatgttgagacggaagactttgaagaaaattcagaagaaattgatgttaatgaaagtgaactttcctcagagattaaatataagacacctcaacctataggagaagtatattttgcagaaacttttgatagtggaaggttggctggatgggtcttatcaaaagcaaagaaagatgacatggatgaggaaatttcaatatacgatggaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 50 - protein, 93 aa, Homo sapiens
MHFQAFWLCLGLLFISINAEFMDDDVETEDFEENSEEIDVNESELSSEIKYKTPQPIGEVYFAETFDSGRLAGWVLSKAKKDDMDEEISIYDG

SEQ ID NO: 51 - DNA, 383 bp, Homo sapiens
  unsure (362)
caacccagaaaccaccacctctcacgccaaagctcacaccttcagcctccaacatgaaggtctccgcagcacttctgtggctgctgctcatagcagctgccttcagcccccaggggctcgctgggccagcttctgtcccaaccacctgctgctttaacctggccaataggaagataccccttcagcgactagagagctacaggagaatcaccagtggcaaatgtccccagaaagctgtgatcttcaagaccaaactggccaaggatatatgtgccgaccccaagaagaagtgggtgcaggattccatgaagtatctggaccaaaaatctccaactccaaagccataaataatcaccattttngaaaccaaaaaaaaaaaaaaa

SEQ ID NO: 52 - protein, 97 aa, Homo sapiens
MKVSAALLWLLLIAAAFSPQGLAGPASVPTTCCFNLANRKIPLQRLESYRRITSGKCPQKAVIFKTKLAKDICADPKKKWVQDSMKYLDQKSPTPKP

SEQ ID NO: 53 - DNA, 1828 bp, Homo sapiens
caagggactagcaggcctagggatacccttcctctatggctccagtgtcccagctgcccccgctgcctaccatggcaggagcatgctccctgccggtgacctgcattttcacagaagcaccctcagaaaccttcagggaaaccccatgctagcggcaactgcaccacactttgaggagagctgggggcagagatgtcgtcgactcaggaaaaatacagggaatcaaaaagctctagacagtgatgctgagagttccaaaagtcaagcagaagaaaaaatcctaggtcagacttatgcagttccctatgaagacgatcattatgcaaaagacccagacattgaagcacccagcaaccagaagtcaagtgaaacgaatgaaaagccaacgacagctcttgccaacacctgtggagagctcgagcccacccataggaaaccctgggggtctcacaccactaccctgaaagcaaaggcctgggacgatgggaaagaggaggcttcggagcagatttttgcaacctgtgatgaaaagaatggggtttgccctccagttcctcgaccatctctgccaggaacacatgcactggttacaattggggggaatctttctttggatgaagatattcagaagtggaccgtggatgatgtgcacagcttcattcgcagccttccaggttgttcagactatgctcaggtatttaaagatcatgcaattgatggagaaactttgccattactcacagaagagcatcttcgaggcactatgggattaaagctagggccggcactaaaaattcagtcacaggtatctcagcatgtgggaagtatgttttacaagaaaactctttcatttcctataagacaagcatttgatcaaccagcagatacatcccctgttctggatcctaattcctggagtgatacaatgaacattttttgtccccaggatacaataattcctaaaggaattgagcgaggtagtatgagaaactaaaagccctcaaggaggaagaatagtcttagccagttttccaaagagcctaggatattacaaaggatgtgtgaggatttcccagtactggatggagaatgagaagagaaagtcagcctttctggggctttcatggaggagacttgcccaaggggcttccctgccagggctgaatgaccccagcaccaaatgactggagacgcatccttaggagcaaatgctaatgaaggggagatttacaaacatcaagaaagctgcagatggatgtttccatgaagaaatctctgaagaaaccaaaagagctgaaactgaaaaaagagctccctcccttttttcactcattttccattcccaaaccctgaaagagataaaggcaacccaatgaaaccacagagaggcagaagagaacgggcagaagtacagaacagaaatggagaagctgaccacgactccatctcttctcattccagatttccagcgtgaagcaggcccaagctagggaaggtgagaagctttaaccttaaattttcgagttttggccaggcgcggtggctcacggctgtaatcccaacactttgggagtctgaggcgggcgratcatgaggtcaggagattaagaccagcttggccaacatggtgaaaccccgtctctactaaaaatacaaaaaaattagctaggcctggtggtgcgcgaatgtagtcccagctactcgggaggctgaagcaggagaattgcttgaacctgggaagcggaggctacagtgagctgagatcgtgccactgcactccagcctgggtgacagagcaggactctgtctcaaaaaaaaaaaaaaaaaa

SEQ ID NO: 54 - protein, 303 aa, Homo sapiens
MLPAGDLHFHRSTLRNLQGNPMLAATAPHFEESWGQRCRRLRKNTGNQKALDSDAESSKSQAEEKILGQTYAVPYEDDHYAKDPDIEAPSNQKSSETNEKPTTALANTCGELEPTHRKPWGSHTTTLKAKAWDDGKEEASEQIFATCDEKNGVCPPVPRPSLPGTHALVTIGGNLSLDEDIQKWTVDDVHSFIRSLPGCSDYAQVFKDHAIDGETLPLLTEEHLRGTMGLKLGPALKIQSQVSQHVGSMFYKKTLSFPIRQAFDQPADTSPVLDPNSWSDTMNIFCPQDTIIPKGIERGSMRN

SEQ ID NO: 55 - DNA, 2110 bp, Homo sapiens
cgcttttttttttttttttgtatctttctagttcattctgtccattgctacttttttataaagaamatttcactaccatatactttctgttccagmcacaggaamctgtttgcaggtccctgaacctaccttcattttctagtgctgtgcatttcctcatttctttcatttggaaagtggtgaraaggtcctctaacttgcttcttgccctcatttctctaagcagagctacaactttgtcctgtctgttcctctggcacttcccatcaccgtgatagcatacttcactgtggctggtttccattcccatttcctctactatagtgagccacttgaggcgagaacccttgtatttccaacattggtgtactcagcatcttgcgtcgaggactcagtaagtattatatttgaattcccactgcaccgctctaattagaattttaaaaatcactttctatstggattgttacacactttttttccccttaattcatttttctccatgtactacccatatgcatcctatataaatttaccaacactcataaaaatcttactcagaaatcttcagaggtttgctaaggatacaatttgattcttacacatttaatgctcaccagctgcttaggcccacaccatttatccaccctgatttgctactgctctttgaaatacaaccagtgtttcagccagactgttttcctgcttctgctccccttctcctcctcccagcacatctgtgaattctttgactggtttaccactcccacactcctccccagcaatgcagatcttctacaccctttaggatctaagctaagtctgcttcccagatatcctcccgaactctgaatgtcctttgatctctcttttctcaaaaaccttttataccttttattcccaaattatcttctctagaaatctgtaagggcaggggaaggaaaggttttacactattctgtcttcggcagaggacttttattattgcttgttatgtacagtattagtcatatcttgctaacactcagtgaactgccctcagctgatcctctgccctgtacctcacctctgctttaccacctttccatccgatttctaaaactgtagtgggactttttaaaaagtgtgtgagagttttaatcctgttgtggtaaactcgcctttctttgcttgctatactgtttctagtccctattttccagataaaaatagcccaggtgagcatggcttgcactatttttaactccaggtactaggagaaaggtttgctctaaaggtgaaatgaagttttatgacgttgcttaggccaaaagtcagttgatgaggttatgaactgcaatagaactttacacatccatgaatctaatggagagggcctgcatatcttatgtccctcacccctaccaatggtgtgtgaatccttcaaaacaaaacagcataatagacataaataaaataaaaagtagttttatttctcagcagtgccttttcagattaggacttggtcttagttgcccttaactctgttcttggtgttggttaacattgtcatcagaagttcagattcaattttagattcagaattcttgacttggtttactgggatgtcagaaccttatttttaattaaagcgaatctattgttgcttaggcagttggcacatagatttggcactgtgagaaaggaaatagtgttcagttgataagggcatcttgaactgaatttcagatagttgtgttacttaatgggtaatgaaataactccagctgaggaacaccttgattaaaaaatctttctctcctcatgccttctccgttagcagaatgaagtcaagtggcaatattttatttataatcatgaatttaattatttaagctatattaaaaaaattgaaacccttagatgttaattaattttaaaaactagtgatggatgcaggtaagctagaatgattggatcaaatctcacacacaaatgagtttattctttaaaaaaaaattttttttttagagacgggktcttgctatgttscccaggatgktcttgaactcatgacctcaagcaatcctcctscctcascctacctgaattaaaaaaaaaaaaaaaaaaa

SEQ ID NO: 56 - protein, 80 aa, Homo sapiens
MLTSCLGPHHLSTLICYCSLKYNQCFSQTVFLLLLPFSSSQHICEFFDWFTTPTLLPSNADLLHPLGSKLSLLPRYPPEL

SEQ ID NO: 57 - DNA, 1353 bp, Homo sapiens
ggagagctgttcagctatggctgctgtcacacattacctgtatctttgccagtttagctggatgctcattcagtctgtgaatttctggtacgtgctggtgatgaatgatgagcacacagagaggcgatatctgctgtttttccttctgagttggggactaccagcttttgtggtgattctcctcatagttattttgaaaggaatctatcatcagagcatgtcacagatctatggactcattcatggtgacctgtgttttattccaaacgtctatgctgctttgttcactgcagctcttgttcctttgacgtgcctcgtggtggtgttcgtggtgttcatccatgcctaccaggtgaagccacagtggaaagcatatgatgatgtcttcagaggaaggacaaatgctgcagaaattccactgattttatatctctttgctctgatttccgtgacatggctttggggaggactaacaacatggcctacagacacttctggatgttggttctctttgtcattttcaacagtctgcagggactttatgttttcatggtttatttcattttacacaaccaaatgtgttgccctatgaaggccagttacactgtggaaatgaatgggcatcctggacccagcacagcctttttcacgcccgggagtggaatgcctcctgctggaggggaaatcagcaagtccacccagaatctcatcggtgctatggaggaggtgccacctgactgggagagagcatccttccaacagggcagtcaggccagccctgatttaaagccaagtccacaaaatggagccacgttcccgtcctctggaggatatggccaggggtcactgatagccgatgaggagtcccaggagtttgatgatttaatatttgcattaaaaactggtgctggtctcagtgtcagtgataatgaatctggtcaaggcagccaggaggggggcaccttgactgactcccagatcgtggagctcaggaggatacccatcgccgacactcacctgtagcacctcactaaccattcgactgagcacactttcatatttgtatcagcttttgtgctaaaactctctaagtacatccacctgtgtaataggaacctgtgaattgtactggatgattaatacaaacgtgattgttgtatttggagtataaattactgattgtatgtgacctgaaaattcactgctataagaaaggtggagtcagtttgtatcagttaataggatkttcatattccaaggatattagttgtttttttaatcatcctatatggctaacattgtttaatgaaagtaataatcaataaagcaatagaatctaaaaaaaaaaaaaaa

SEQ ID NO: 58 - protein, 189 aa, Homo sapiens
MALGRTNNMAYRHFWMLVLFVIFNSLQGLYVFMVYFILHNQMCCPMKASYTVEMNGHPGPSTAFFTPGSGMPPAGGEISKSTQNLIGAMEEVPPDWERASFQQGSQASPDLKPSPQNGATFPSSGGYGQGSLIADEESQEFDDLIFALKTGAGLSVSDNESGQGSQEGGTLTDSQIVELRRIPIADTHL

SEQ ID NO: 59 - DNA, 2949 bp, Homo sapiens
gttgctgttgcgcgggatggtggatgacttgtcaaactcgggcgggccctcgctgtccgcatccccattcacggagtagcagtcgtagtcggagcctgggggcacgggacacagtgaggcccagggcccaggtggccccttgcccccagcccaccagggtgagcacagagggggaaggacggggccctcctggatggctaagtcccagctgtccctggtcccaccccagccccgcgggcctgccttgggaggggatggtgtcctcagagcaggagggcgtggtggtctgcgtgctgtagccgctggagtactgcagcgagtcccggctgctcttctggtgctccaggctcaggccccgcgtcagcaccatggccaggtcactggcggcgggggacacctcctcaccgtgctgagggtgggaaagtgcaggctgaagccttgcgcccccaatcccctctgcccaaattccaggcagccctgccaggtggttggagccggccctgaggccactggctgggcctcctgggactgacctgggcgtgaaggggggccctggccagcctaccttggctgcgatggtggcaggggacatccggggtcgcggtgcctcttccccgctggggcccagggtgcccccactggcagggcctggctctgtgtctcgcaggagctccactcggtccttcctccgctgcagagtggcgcctgagggctgctcatgggagccgaccttggaccagtcctgcagggagggtgtggcaggtcagggggaccactggccctaccacctatggcccgggggcccgtcactgtggctgaggctcagggtgtccagggctatcagggggtctctggttcgcaatcacagccaagggcagagcatggagtgtgactgtggcccctcagtcccaccaggcccaggggtaagatgtggctgtgtgggcagtgactgtaggaaatggccaggtggcttgtctcttggggacaggtgctgtgtgcacgcacccctggctgaacccaggccctttgtgcagtaatggcaccaggtggggagggcatggatgggaggggcagctggtgactgtcctggggccgggctgggagcactgaccgaggtgggggagctgcactcgctaacggactggcaggtttccgaggcctcggaagatgcagagctggaggacttctgccatgcagaggccagcaggcggttgcagacagaggggccggcaggcagcaaggaggagaggagagaacagttttggtcacagagctggcatgcaggggggcggttcaattcagcgccttgcccagcacccaccccccctaccccagggccacggccgctgagtgtctgcagcagcacacagcagcccagagtgcacacggtgaacgctgacccacctgacagcccagccctgcgcattttgaagactgaccacaggactcactgctgacggcctggatatggccaccgaggggaaaaagagatactaagggcggcagctgggtgtttggtctgagcagttgggttgatggtgtcacagttttctcagacaggacacgggggaagtgtgaagccccgagtgccacgctgagcactgagctgagctgcctctgaggtctctgcatggatgcaatgagggccgtcgtattcataagctgagtgcagggagaagttggggttggagccatcagtgcagtggtcttggggtgggatgaggccaccaggcagcgagtgcagttagacaagattcaagggccaggcccgtggctgctgatgcgcatcaggtgggagggaagaggagagtcccgtaaaggagagcctgctctgcggagaaggggggcagctgtgctgatgctgctgggtggggccatcggcttggggcacagggtgtccatggagaccccgaggagaactgctaccatagaagggggtgggggaaggctgttttaaagtggactgaagtatgaaagggacacacctgctctgggcagtgggctggactaggtgttctgcgaggccccccactcctaaacaactacaaagaataaagaaaaaagcagggtagacacaggcccatgaaatcctgagcatgtgtgaccggcagggttccctggggcagacgccagcttcagcacagcagtggggacgtggatcacggtgaggtgttggcctgggaatgagccaagttaagccagggcagcccccttcctccagctctctgaatcattctggatttagatccccagatttcctgcagattcgaatgaaccaagatcaccaaaataaaaggaaaccaggcaccacctgtgaggatttggaggaaataacaaatgcagacttttggcaatatgacggttaaagtaaaaaaactcaatggaaaaagtatttggaaaactcagtgaaaaagttcaacacttgctcatgaaaaacaatacaaagacacaagctgctagaaaggaagctaatctgataaagaatatctacaaatgcattgatacttttccctctgaggttgggagaaagacaaggatgcccactgtcatcactcctattcagcactggaaatcttagccagtgcaataaagcaaggaaaagaaataaaaaacaaatggattggaaagaaaagaaagaaggccaagtgtggtggctcacacctgtaatcccagcactttgggaggctgatgcataaggatcacttgagcccaggagttcaagaccagcctaacatagtgagacccctgactctacaaaaaattaaagttagccaggtgtggtggcacatgcctgtggtcccagctacacaggaagctgaggcaggaggatatcttgagcctaggaattcaaggctgcagtgagctgtgatcacaccactgaactcctgaactccagtgtgcgtgacagagcaagactctgtctcaaaaaaaaaaaaaaaaaaaaaaaaa

SEQ ID NO: 60 - protein, 153 aa, Homo sapiens
MVAGDIRGRGASSPLGPRVPPLAGPGSVSRRSSTRSFLRCRVAPEGCSWEPTLDQSCREGVAGQGDHWPYHLWPGGPSLWLRLRVSRAIRGSLVRNHSQGQSMECDCGPSVPPGPGVRCGCVGSDCRKWPGGLSLGDRCCVHAPLAEPRPFVQ

SEQ ID NO: 61 - DNA, 298 bp, Homo sapiens
ggaawaagaagagaaagccaaggaagacaagggcaaacaaaagttgaggcagcttcacacacacagatacggagaaccagaagtgccagagtcagcattctggaagaaaatcatagcatatcaacagaaacttctaaactattttgctcgcaacttttacaacatgagaatgttagccttatttgtcgcatttgctatcaatttcatcttgctcttttataaggtctccacttcttctgtggttgaaggaaaggagctccccacgagaagttcaagtgaaaatgccaaagtgacaagc

SEQ ID NO: 62 - protein, 45 aa, Homo sapiens
MRMLALFVAFAINFILLFYKVSTSSVVEGKELPTRSSSENAKVTS

SEQ ID NO: 63 - DNA, 165 bp, Homo sapiens
  unsure (22)
  unsure (56)
  unsure (122)
ggaaacattttgctgattttgngaattgccagcgttgtgtgttttctgggagcatngaagctctgtttcggaagagctgtttcctccccccaccttttgtatttactttgagactaaagacngaagaataatctaaattcatactcagacaaaaaaaaaaaaaaa

SEQ ID NO: 64 - DNA, 2412 bp, Homo sapiens
tttttttttttttttttttttttttttttttttttttttttttttttttttttttttttttttttttttttttttttttggaaataggatggattttattttttacttatttatttatttatttatttatttatttatttatttatttatatattttttttgagacagagtcccactgtgttgcccaggctggattactgtggcacaatctcggcaacgagagtgaatctgtgtctcaaaaaaaagtacaaaaattagccagacatggtggcacacgcctgcagtcacagctacctgggcaggcgaggcaggagaactgcttgaacccaggaggcagatgctgcagtaagccaagatcgtgccactgactccagcctgggtgacagagctcaaaaaaaaaaaaaaatgagataaaacatagatacagaaaaccacaaaggaaaaacatagcatattgaatcatcacaaggcagccacctcttcatagccacacctggcccctggccaccactgacctgtgctccatcgccagaattccgttgtctcaggaatgttcaatagatggaatcctgtgtggcctgagatgagtgtctttcatgccgcgtgacacccttgaggcccgtgcaactgttggtatgtcaacagttagctgcttctcattgctgagtggcgattggtcctgtcatggtttattcagccatgtggtggatggctacttgtcttytaagccacttgccttctgatcgctggactgactctctcgccctctcttggtgcagtcctcaggaggctcggtcacactctccaagagcacagccatcatctcccacggtaccacaggcctggtcacatgggatgccgccctctaccttgcagaatgggccatcgagaacccggcagccttcattaacaggtgacctcggggcacagggcagggcaccgaggcaggcttaccctggtgcagtcgaaaacacggtcccctttcctcccgccaggactgtcctagagcttggcagtggtgccggcctcacaggccttgccatctgcaagatgtgccgcccccgggcatacatcttcagcgaccctcacagccgggtcctcgagcagctccgagggaatgtccttctcaatggcctctcattagaggcagacatcactggcaacttagacagccccagggtgacagtggcccagctggactgggacgtagcaatggtccatcagctctctgccttccagccagatgttgtcattgcagcaggtaatgcccagccccgggcatcctgtgcaggcggtgtccttgcagctctacccagctcttggctctgggaaaagggaacaatggacactgtcgggcatggacgtgatggggcttccagaagagttactctgggcctccagggtgacatcaaaggacaggggtgcctcttaaggtgaccttcaagccacagccctgttgttggagacaggcatactcccgttacagtcgtcaccacatggctctgtcccagagccatgccctgtgtccttcagagaccgcaggaggaaaacaaccacttttggtacgaggtcagggcccttgagagaaggtgctgtttggctgggccaccaaaaacccctcacccctgtgagcacactcagtcccctctctggtggaacagagctctgcctgtagtcctgggtcccagccctgaaacccacaggtccagcggtggccagggacacaggcccacccctgcaagccagcagaccaatcggcagacacctgaaacacgaagttcacggcagggtcaggctttctgtcattgaaagccctctagacaggccgagaaccagagctggttttttaaggacaccagtgagtctggagatttttttcttttgcttcggtcttttgcagctttctctaataagggttctcctttttcacccaaataattgcctttccatctaatggcccaaatggtcaaatggcatctaatagtctcatatgaccgctgcctctctggcctcgccctgctgctgaggtcagcatgaaatggaactttccacttgtccctttcagtaacctgaaactttcactgtagacgtgatgtattgcccagaagccatcgtgtcgctggtcggggtcctgcagaggctggctgcctgccgggagcacaagcgggctcctgaggtatacgtggcctttaccgtccgcaacccagagacgtgccagctgttcaccaccgaggtaggtgagcccccacgcccacccgggcctacatggtgcccgagctgtccctgcaggactccagtgagagtgaaagaactgggcgctggggaaaagctaggatgctccacactcccacactatgcggggaactcgggcagaggccggtgagcagggtgggctcggggcgtggggggctt

SEQ ID NO: 65 - protein, 250 aa, Homo sapiens
MVYSAMWWMATCLLSHLPSDRWTDSLALSWCSPQEARSHSPRAQPSSPTVPQAWSHGMPPSTLQNGPSRTRQPSLTGDLGAQGRAPRQAYPGAVENTVPFPPARTVLELGSGAGLTGLAICKMCRPRAYIFSDPHSRVLEQLRGNVLLNGLSLEADITGNLDSPRVTVAQLDWDVAMVHQLSAFQPDVVIAAGNAQPRASCAGGVLAALPSSWLWEKGTMDTVGHGRDGASRRVTLGLQGDIKGQGCLLR

SEQ ID NO: 66 - DNA, 2412 bp, Homo sapiens
cagttttagaaagaagcaccttgctggatagatttggaggttttcttttggaaattcagattccatatgtgttttttgcatctgaaggacttcttaatactccagacatacttcagctgctagaatccaactataacatctcactagtagagagaggctgcagtgagtcattgaaactctttggaagttcagagtgttatgtagtggtgacaattgatgaacacactgccataattttgcaggatctagaagaattgaattgtgagaaggcatcagacaatatcattatgaggctgatggcattatcattacagtacagatattgttggataattttatataccaaagaaacattaaattcagagtatccgcttacagaaaagacacttcatcacctagcactgatttatgcagctttggtttcatttgggctaaactctgaagaactggatgtaaagcttataattgccccaggagtagaagcaactgccttgataattcgacaaattgctgaccacagtttaatgacctcaaagagagatcctcatgaatggttggataaatcctggcttaaagtttcaccatctgaggaagaaatgtacttacttgattttccatgtattaacccattggtggctcagctcatgctaaataaaggaccttcactgcattggatattattagcaactctgtgtcaacttcaggaactcctacctgaagtcccagaaaaagtgttaaagcatttttgtagcatcacttccctattcaagattggttcttcttccataacaaaatcaccgcaaatttcgtcacctcaggaaaataggaatcagattagtaccttgtcttctcaaagttcagcttctgatttagactctgtcattcaagaacataatgaatattatcagtatttaggattaggagagacagtgcaggaagacaaaaccaccactttgaatgacaactcttccattatggaactaaaagaaatctcaagttttttaccacctgtgacttcatacaatcagaccagctactggaaagactccagctgtaaatctaatatagggcagaatactccttttctaattaatatagaatcaaggagaccggcttataactcctttctaaaccacagtgattcagagtcagatgtcttttctttgggtctaacacaaatgaactgtgaaactataaaatcaccaactgacactcagaagagagtgtcagttgtcccccgttttataaattctcagaaaaggagaacacatgaagcaaaaggtttcataaataaagatgtatcggaccctatcttttcactagagggcactcaatctcctcttcattggaactttaagaaaaatatatgggaacaagagaatcacccgttcaacttacaatatggtgcacagcagactgcatgtaacaaattgtactctcagaaaggtaatttattcactgatcagcaaaaatgtctatcagatgagtctgaaggcctcacatgtgaaagttcaaaagatgagactttctggagagaattaccatctgtccccagtttggatttatttcgtgcttctgattctaatgcaaatcaaaaagaattcaacagcctttatttctaccaaagagctggaaaaagtttaggacagaaaaggcaccatgaatcttcatttaactcaggagacaaggaatcattaacaggttttatgtgctcacaactaccacaattcaaaaaacgacgtctagcatatgaaaaagtccctggtagagttgatgggcagactcggctgaggtttttttgaaggaggagaagagcaatgttacatgccatattccactgtttttgatgctaatccactagcgcaattatttagatttgctcatacactaaagaaaacacaattgttcatatgtctcagtatttctgtattaaatattcataatatgtattctgccctatggtttgcatctttgtaagttaaatattctaatttatcaattagcagaataattatcataagatccaaaatgtcttccagacacccctgcacacaggccatttaaatgagtctccatcacagtctgaccctttgagtcaggaagtgaagatcatcacagttaaccctcccacatcaagaaagttaaaacytaggacaaaattgaagttagaaaayttccaacttaaagtatcattttctgtaaacacaatttaagaacaaattactaagaggaaatatttgcaacccagataataggaaaaaaagtttacatttctcatatataaagaattcctacaaattgatagaaagaagacaacctgatagaagaacgggcaaaatatatgaacagatatttcctcagaaaaaaacaaaaattgtcaataaacatttgaaacacaaaaaaaaaaaaaaa

SEQ ID NO: 67 - protein, 504 aa, Homo sapiens
MRLMALSLQYRYCWIILYTKETLNSEYPLTEKTLHHLALIYAALVSFGLNSEELDVKLIIAPGVEATALIIRQIADHSLMTSKRDPHEWLDKSWLKVSPSEEEMYLLDFPCINPLVAQLMLNKGPSLHWILLATLCQLQELLPEVPEKVLKHFCSITSLFKIGSSSITKSPQISSPQENRNQISTLSSQSSASDLDSVIQEHNEYYQYLGLGETVQEDKTTTLNDNSSIMELKEISSFLPPVTSYNQTSYWKDSSCKSNIGQNTPFLINIESRRPAYNSFLNHSDSESDVFSLGLTQMNCETIKSPTDTQKRVSVVPRFINSQKRRTHEAKGFINKDVSDPIFSLEGTQSPLHWNFKKNIWEQENHPFNLQYGAQQTACNKLYSQKGNLFTDQQKCLSDESEGLTCESSKDETFWRELPSVPSLDLFRASDSNANQKEFNSLYFYQRAGKSLGQKRHHESSFNSGDKESLTGFMCSQLPQFKKRRLAYEKVPGRVDGQTRLRFF

SEQ ID NO: 68 - DNA, 1934 bp, Homo sapiens
  unsure (21)
  unsure (24)
caattgcaganttngaattcggctttcatggcatacggccttcatggcctaggggaggaagttgccttgtactgtgccaaatatcttcctgatatcatcaaagatcagaaggcctacaaggaaggcaagctacagaaggctttaaaagatgccttcttggctattgacgccaaattgaccactgaagaagtcattaaagagctggcacagattgcagggcgacccactgaggatgaagatgaaaaagaaaaagtagctgatgaagatgatgtggacaatgaggaggctgcactgctgcatgaagaggctaccatgactattgaagagctgctgacacgctacgggcagaactgtcacaagggccctccccacagcaaatctggaggtgggacaggcgaggaaccagggtcccagggcctcaatggggaggcaggacctgaggactcaactagggaaactccttcacaagaaaatggccccacagccaaggcctacacaggcttttcctccaactcggaacgtgggactgaggcaggccaagttggtgagcctggcattcccactggtgaggctgggccttcctgctcttcagcctctgacaagctgcctcaagttgctaagtccaagttctttgaggacagtgaggatgagtcagatgaggcggaggaagaagaggaagacagtgaggaatgcagcgaggaacacgatggctacagcagtgaggaggcagagaatgaggaagatgaggatgacaccgaggaggctgaagaggacgatgaagaagaagaagaagagatgatggtgccagggatggaaggcaaagaggagcctggctctgacagtggtacaacagcggtggtggccctgatacgagggaagcagttgattgtagccaacgcaggagactctcgctgtgtggtatctgaggctggcaaagctttagacatgtcctatgatcacaaaccagaggatgaagtagaactagcacgcatcaagaatgctggtggcaaggtcaccatggatgggcgagtcaacgggggcctcaacctctccagagccattggggaccacttctataagagaaacaagaacctgccacctgaggaacagatgatttcagcccttcctgacatcaaggtgctgactctcactgacgaccatgaattcatggtcattgcctgtgatggcatctggaatgtgatgagcagccaggaagttgtagatttcattcaatcaaagatcagccagcgtgatgaaaatggggagcttcggttattgtcatccattgtggaagagctgctggatcagtgcctggcaccagacacttttggggatggtacagggtgtgacaacatgacctgcatcatcatttgcttcaagccccgaaacacagcagagctccagccagagagtggcaagcgaaaactagaggaggtgctctttactgagggggctgaagaaaatggcaacagcgacaagaagaagaaggccaagcgagactagcagtcatccagacccctgcccacytagaytgttttytgagccytccggacctgagaytgagttttgtytttttcctttagccttagcagtgggtatgaggtgtgcagggggagctgggtggcttcamtccgcccattccaaagagggytctccytccacactgcagccgggagcytytgmtgtccttcccagccgcytytgytcctcgggytcatcaccggttctgtgcctgtgytctgttgtgttggagggaaggactggcggttctggtttttactctgtgaactttatttaaggacattyttttttattggcggctccatggccctcggccgcttgcacccgytytytgttgwacactttcaatcaacactttttcagactaaaggccaaaacctaaaaaaaaaaaaaaaaaaaaaaaaaaaaaaaaa

SEQ ID NO: 69 - protein, 491 aa, Homo sapiens
MAYGLHGLGEEVALYCAKYLPDIIKDQKAYKEGKLQKALKDAFLAIDAKLTTEEVIKELAQIAGRPTEDEDEKEKVADEDDVDNEEAALLHEEATMTIEELLTRYGQNCHKGPPHSKSGGGTGEEPGSQGLNGEAGPEDSTRETPSQENGPTAKAYTGFSSNSERGTEAGQVGEPGIPTGEAGPSCSSASDKLPQVAKSKFFEDSEDESDEAEEEEEDSEECSEEHDGYSSEEAENEEDEDDTEEAEEDDEEEEEEMMVPGMEGKEEPGSDSGTTAVVALIRGKQLIVANAGDSRCVVSEAGKALDMSYDHKPEDEVELARIKNAGGKVTMDGRVNGGLNLSRAIGDHFYKRNKNLPPEEQMISALPDIKVLTLTDDHEFMVIACDGIWNVMSSQEVVDFIQSKISQRDENGELRLLSSIVEELLDQCLAPDTFGDGTGCDNMTCIIICFKPRNTAELQPESGKRKLEEVLFTEGAEENGNSDKKKKAKRD

SEQ ID NO: 70 - DNA, 1499 bp, Homo sapiens
cggccttcatggcctactttcctcgcacagccaggcggcccctgctcgagtcccgcgtcgccatggccgcggttcccgagttgctgcagcagcaggaggaggaccgcagcaagctgagatctgtatctgtggacctgaatgttgatccctcgcttcagattgacatacctgatgcgctcagtgagagagacaaagtcaaatttacagtgcacacaaagaccacactgcccacgtttcagagcccagagttttctgttacaaggcaacatgaagactttgtgtggctacatgacactcttattgaaacaacagactatgctgggcttattattccacctgctcctacgaagcccgactttgatggtcctcgagagaagatgcagaaactgggagaaggtgaagggtctatgaccaaagaagaatttgcccagatgaaacaagaactggaagctgagtatctcgctgtgtttaagaagactgtgtcctcccatgaagtctttcttcagcggctttcttctcaccctgttctcagtaaagatcgcaactttcatgttttcctggaatatgatcaggatctaagtgttaggcggaaaaatactaaagaaatgtttggtggcttcttcaaaagtgtggtgaaaagtgctgatgaagtcctttttactggagttaaggaggtagatgacttctttgagcaagagaagaacttccttattaactattacaataggatcaaagattcttgtgtgaaagctgacaaaatgaccagatctcataaaaatgttgccgatgactatatccacaccgcagcctgcttacatagcctggctttagaagagcccacagtcatcaaaaagtacctattgaaggttgctgagctatttgaaaaactaaggaaagtagagggtcgagtttcatcagatgaagatttgaagctaacagagctcctccgatactacatgctcaacattgaagctgctaaggatctcttatacagacgcaccaaagccctcattgactatgagaactcaaacaaagctctggataaggcccggttaaagagcaaagacgtcaagttggctgaggcacaccagcaggagtgctgccagaaatttgaacaactttccgaatctgcaaaagaagaactgataaatttcaaacggaagagagtggcagcatttagaaagaatctaattgaaatgtctgaactggaaataaaacatgccaggaacaatgtctcccttttgcagagctgtattgacttgttcaagaataactgatatgccttcactcagaagaaaagaaatgaatgtgaaagaaagccaagcatcacttgcacttaaatcattaccacggaagatatattagcttcaactttagtttaaaattatgtgaataaatattttgatttctacaaatcttaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 71 - protein, 422 aa, Homo sapiens
MAYFPRTARRPLLESRVAMAAVPELLQQQEEDRSKLRSVSVDLNVDPSLQIDIPDALSERDKVKFTVHTKTTLPTFQSPEFSVTRQHEDFVWLHDTLIETTDYAGLIIPPAPTKPDFDGPREKMQKLGEGEGSMTKEEFAQMKQELEAEYLAVFKKTVSSHEVFLQRLSSHPVLSKDRNFHVFLEYDQDLSVRRKNTKEMFGGFFKSVVKSADEVLFTGVKEVDDFFEQEKNFLINYYNRIKDSCVKADKMTRSHKNVADDYIHTAACLHSLALEEPTVIKKYLLKVAELFEKLRKVEGRVSSDEDLKLTELLRYYMLNIEAAKDLLYRRTKALIDYENSNKALDKARLKSKDVKLAEAHQQECCQKFEQLSESAKEELINFKRKRVAAFRKNLIEMSELEIKHARNNVSLLQSCIDLFKNN

SEQ ID NO: 72 - DNA, 2265 bp, Homo sapiens
ggctgggctcgggctcagctcgactgggctcggcgggcggcggcggcggcgcccgcggctggcggargarggagggcgagggcgggcgcgggccggcgggcgggcggaararggaggaraggcgcggggagccaggcctcggggcctcggagcaaccacccgagcagacggagtacacggagcagcggccccggccccgccaacgctgccgccgggatgctccaraccttgtatgattacttctggtgggaacgtctgtggctgcctgtgaacttgacctgggccgatctagaagaccgagatggacgtgtctacgccaaagcctcagatctctatatcacgctgcccctggccttgctcttcctcatcgttcgatacttctttgagctgtacgtggctacaccactggctgccctcttgaacataaaggagaaaactcggctgcgggcacctcccaacgccaccttggaacatttctacctgaccagtggcaagcagcccaagcaggtggaagtagagcttttgtcccggcagagcgggctctctggccgccaggtagagcgttggttccgtcgccgccgcaaccaggaccggcccagtctcctcaagaagttccgagragccagctggagattcacattttacctgattgccttcattgccggcatggccgtcattgtggataaaccctggttctatgacatgaagaaagtttgggagggatatcccatacagagcactatcccttcccmgtattggtactacatgattgaactttccttctactggtccctgctcttcagcattgcctctgatgtcaagcgaaaggatttcaaggaacagatcatccaccatgtggccaccatcattctcatcagcttttcctggtttgccaattacatccgagctgggactctaatcatggctctgcatgactcttccgattacctgctggagtcagccaagatgtttaactacgcgggatggaagaacacctgcaacaacatcttcatcgtcttcgccattgtttttatcatcacccgactggtcatcctgcccttctggatcctgcattgcaccctggtgtacccactggagctctatcctgccttctttggctattacttcttcaattccatgatgggagttctacagctgctgcatatctyctgggcctacctcattttgcgcatggcccacaagttcataactggaaagctggtagaagatgaacgcagtgaccgggaagaaacagagagctcagagggggaggaggctgcagctgggggaggagcaaagagccggcccctagccaatggccaccccatcctcaataacaaccatcgtaagaatgactgaaccattattccagctgcctcccagattaatgcataaagccaaggaactaccctgctccctgcgctatagggtcactttaagctctggggaaaaaggagaaagtgagaggagagttctctgcatcctccctccttgcttgtcacccagttgcctttaaaccaaattctaaccagcctatccccaggtagggggacgttggttatattctgttagagggggacggtcgtattttcctccctacccgccaagtcatcctttctactgcttttgaggccctccctcagctctctgtgggtaggggttacaattcacattccttattctgagaatttggccccagctgtttgcctttgactccctgacctccagagccagggttgtgccttattgtcccatctgtgggcctcattctgccaaagctggaccaaggctaacctttctaagctccctaacttgggccagaaaccaaagctgagcttttaactttctccctctatgacacaaatgaattgagggtaggaggagggtgcacataacccttaccctacctctgccaaaaagtgggggctgtactggggactgctcggatgatctttcttagtgctacttctttcagctgtccctgtagcgacaggtctaagatctgactgcctcctttctctggcctcttcccccttccctcttctcttcagctaggctagctggtttggagtagaatggcaactaattctaatttttatttattaaatatttggggttttggttttaaagccagaattacggctagcacctagcatttcagcagagggaccattttagaccaaaatgtactgttaatgggtttttttttaaaattaaaagattaaataaaaaatattaaataaaacatgaaaaaaaaaaaaaaaa

SEQ ID NO: 73 - protein, 380 aa, Homo sapiens
  UNSURE (135)
  UNSURE (179)
  UNSURE (318)
MLQTLYDYFWWERLWLPVNLTWADLEDRDGRVYAKASDLYITLPLALLFLIVRYFFELYVATPLAALLNIKEKTRLRAPPNATLEHFYLTSGKQPKQVEVELLSRQSGLSGRQVERWFRRRRNQDRPSLLKKFRXASWRFTFYLIAFIAGMAVIVDKPWFYDMKKVWEGYPIQSTIPSXYWYYMIELSFYWSLLFSIASDVKRKDFKEQIIHHVATIILISFSWFANYIRAGTLIMALHDSSDYLLESAKMFNYAGWKNTCNNIFIVFAIVFIITRLVILPFWILHCTLVYPLELYPAFFGYYFFNSMMGVLQLLHIXWAYLILRMAHKFITGKLVEDERSDREETESSEGEEAAAGGGAKSRPLANGHPILNNNHRKND

SEQ ID NO: 74 - DNA, 377 bp, Homo sapiens
  unsure (204)
  unsure (330)
ggcagaatgggactccaagcctgcctcctagggctctttgccctcatcctctctggcaaatgcagttacagcccggagcccgaccagcggaggacgctgcccccaggctgggtgtccctgggccgtgcggaccctgaggaagagctgagtctcacctttgccctgagacagcagaatgtggaaagactctcggagctggtgcangctgtgtcggatcccagctctcctcaatacggaaaatacctgaccctagaaaaatgtggctgatctggtgaggccatccccactgaccctccacacggtgcaaaaatggctcttggcagcccggancccaaaaattgccattctgtgatcacacaggaactttctgacttgct

SEQ ID NO: 75 - protein, 86 aa, Homo sapiens
  UNSURE (66)
MGLQACLLGLFALILSGKCSYSPEPDQRRTLPPGWVSLGRADPEEELSLTFALRQQNVERLSELVXAVSDPSSPQYGKYLTLEKCG

SEQ ID NO: 76 - DNA, 437 bp, Homo sapiens
tgcttccttgggggtgagttttcattcactatgtggggagggaccttacgggaaaacccatgttgtaaggttcccccaaattcctaccagctttccacaggccttggccccccatgtggactttgtggggggactgcaccgtttttcccccaacatcatccctgaggcaacgtcctgagccgcaggtgacagggactgtaggcctgcatctgggggtaaccccctctgtgatccgtaagcgatacaacttgacctcacaagacgtgggctctggcaccagcaataacagccaagcctgtgcccagttcctggagcagtatttccatgactcagacctggctcagttcatgcgcctcttcggtggcaactttgcacatcaggcatcagtagcccgtgtggttggacaacagggccggggccggccggcgcatctcgag

SEQ ID NO: 77 - DNA, 907 bp, Homo sapiens
  unsure (116)
  unsure (270)
  unsure (307)
  unsure (419)
  unsure (719)
  unsure (752)
  unsure (764)
  unsure (781)
  unsure (786)
  unsure (894)
caatgggggaaagtcataattcaacctgaaaacatggattgtaagaggaaataaaaaaatcaaacagtatgttttaagtttcccttttgatactgtgtttcagggtaagtgacagnttctgcaaaccaagactcagtcctgattataaaggatttttaaaattacattattaaaaatatgtatttattcttctttcactttatctattttccaaagcctctttcaagtaaactgtgaagtgcctgagtaccagtgaccatgacgtcacantttcttttatctcaagcactcattgttgtttgcattngcccaaagtgctggctggctccgaaggccagttcccacaagaatgaattcggaaagacttcatccttgtgtttttccttctcttttaattccatgcaacgaggtcagctttngcaacaaggtggggttttatttttttggtgcatgmcatcaaatactctaacgagacatttttaatgaaagayttaaaccagataggccacaatgaaccaaattagaaatytgaacatgtcaccacttgcagcataaaggaatataaaagggcagagcaaagtcttttttcctaaggtgaatatttctaaggtaagtattcatttgtaaaagttttttttttycatcatgtctgaaaacyywttaycacscggtkagtattacaacraracatcccttggttaaaaaaaaaaaataccatnttgcaattcagcacacacctgcagctggtgtgntcatccaaaccnatcagtaggctaagagnatttnaaattccatacatatgagtttaggtattaatgccgattacacagtacacagtacagagggaggtccctatatccacacacacacacaccccatccagcatttacaccnaaagccttaccct

SEQ ID NO: 78 - protein, 74 aa, Homo sapiens
  UNSURE (4)
  UNSURE (17)
  UNSURE (54)
  UNSURE (66)
MTSXFLLSQALIVVCIXPKCWLAPKASSHKNEFGKTSSLCFSFSFNSMQRGQLXQQGGVLFFWCMXSNTLTRHF

SEQ ID NO: 79 - DNA, 371 bp, Homo sapiens
  unsure (151)
  unsure (175)
  unsure (204)
atcaaatcaccttggagtggtcaccagggggacagggagccccccaccaatgtatcaatgggtgatttatgatgccttctgccctttggcgagtgaatgggtttcccataggggaagttggcctccctccgtgagctttggaaatgttttntaatagacacagggaggccagttntgtttcagagcaattatcttcccaaattntctgttctggtgttggaactgtgtgccctggtttctgttttcctttctactgctgtaattctctgtctcatcatccttctcttttgtttccatagccttttataatgcatatatgatgctgtgaacagaaataaattatttatacaatcaaaaaaaaaaaaaaaaaa

SEQ ID NO: 80 - DNA, 740 bp, Homo sapiens
  unsure (4)
  unsure (29)
  unsure (469)
  unsure (472)
  unsure (480)..(481)
atangtatttattcttctttcactttatntattttccaaagcctctttcaagtaaactgtgaagtgcctgagtaccagtgaccatgacgtcacactttcttttatctcaagcactcattgttgtttgcatttgcccaaagtgctggctggctccgaaggccagttcccacaagaatgaattcggaaagacttcatccttgtgtttttccttctcttttaattccatgcaacgaggtcagcttttgcaacaaggtggggttttatttttttggtgcatgccatcaaatactctaacgagacatttttaatgaaagacttaaaccagataggccacaatgaaccaaattagaaatctgaacatgtcaccacttgcagcataaaggaatataaaagggcagagcaaagtcttttttcctaaggtgaatatttctaaggtaagtattcatttgtaaaagttttttttttccancangtctgaannctttttaccacgcggtgagtattacaacaaaacatcccttggttaaaaaaaaaaaataccatcttgcaattcagcacacacctgcagctggtgtgctcatccaaaccaatcagtaggctaagagaatttaaaattccatacatatgagtttaggtattaatgccgattacacagtacacagtacagagggaggtccctatatccacacacacacacaccccatccagcatttacaccaaaagccttaccctttaaacac

SEQ ID NO: 81 - protein, 74 aa, Homo sapiens
MTSHFLLSQALIVVCICPKCWLAPKASSHKNEFGKTSSLCFSFSFNSMQRGQLLQQGGVLFFWCMPSNTLTRHF

SEQ ID NO: 82 - DNA, 280 bp, Homo sapiens
  unsure (63)
  unsure (116)
gcgagtgaatgggtttcccataggggaagttggcctccctccgtgagctttggaaatgttttntaatagacacagggaggccagttctgtttcagagcaattatcttcccaaattntctgttctggtgttggaactgtgtgccctggtttctgttttcctttctactgctgtaattctctgtctcatcatccttctcttttgtttccatagccttttataatgcatatatgatgctgtgaacagaaataaattatttatacaatcaaaaaaaaaaaaaaa

SEQ ID NO: 83 - DNA, 2226 bp, Homo sapiens
catctcagcaggtcttaactgcctcattttcttcaggaggatctgcacttcatccacaggcacaggggcagagccagggtcagccatcctcaagtagcttaacaggggtttcatcttcccaacccatacaacatcctcagcagcagggaatacagcagacagcccctcctcaacagacagtgcagtattcactttcacagacatcaacctccagtgaggccactactgcacagccagtgagtcagcctcaagctccacaagtcttgcctcaagtatcagctggaaaacagcttccagtttcccagccagtaccaactatccaaggcgaacctcagatcccagttgcgacacaaccctcggttgttccagtccactctggtgctcatttccttccagtgggacagccgctccctactcccttgctccctcagtaccctgtctctcagattcccatatcaactcctcatgtgtctacggctcagacaggtttctcatcccttcccatcacaatggcagctggcattactcagcctctgctcacgttggcttcatctgctacaacagctgcgatcccgggggtatcaactgtggttcctagtcagcttccaacccttctgcagcctgtgactcagctgccaagtcaggttcacccacagctcctacaaccagcagttcagtccatgggaataccagctaaccttggacaagctgctgaggttccactttcctctggagatgttctgtaccagggcttcccacctcgactgccaccacagtacccaggagattcaaatattgctccctcttccaacgtggcttctgtttgcatccattctacagtcctatcccctcccatgccgacagaagtactggctacacctgggtactttcccacagtggtgcagccttatgtggaatcaaatcttttagttcctatgggtggtgtaggaggacaggttcaagtgtcccagccaggagggagtttagcacaagcccccactacatcctcccagcaagcagttttggagagtactcagggagtctctcaggttgctcctgcagagccagttgcagtagcacagccccaagctacccagccgaccactttggcttcctctgtagacagtgcacattcagatgttgcttcaggtatgagtgatggcaatgagaacgtcccatcttccagtggaaggcatgaaggaagaactacaaaacggcattaccgaaaatctgtaaggagtcgctctcgacatgaaaaaacttcacgcccaaaattaagaattttgaatgtttcaaataaaggagaccgagtagtagaatgtcaattagagactcataataggaaaatggttacattcaaatttgacctagatggtgacaaccccgaggagatagcaacaattatggtgaacaatgactttattctagcaatagagagagagtcgtttgtggatcaagtgcgagaaattattgaaaaagctgatgaaatgctcagtgaggatgtcagtgtggaaccagagggtgatcagggattggagagtctacaaggaaaggatgactatggcttttcaggttctcagaaattggaaggagagttcaaacaaccaattcctgcgtcttccatgccacagcaaataggcattcctaccagttctttaactcaagttgttcattctgcgggaaggcggtttatagtgagtcctgtgccagaaagccgattacgagaatcaaaagttttccccagtgaaataacagatacagttgctgcctctacagctcagagccctggaatgaacttgtctcactctgcatcatcccttagtctacaacaggccttttctgaacttagacgtgcccaaatgacagaaggacccaacacagcacctccaaactttagtcatacaggaccaacatttccagtagtacctcctttcttaagtagcattgctggagtcccaaccacagcagcagccacagcaccagtccctgcaacaagcagccctcctaatgacatttccacatcagtaattcagtctgaggttacagtgcccactgaagaggggattgctggagttgccaccagcacaggtgtggtaacttcaggtggtttccccataccacctgtgtttgaatcaccagtactttccagcgtagtttcaagtatcacaatacctgcacgacaacacataaaaaaaaaaaaaaaaaaaaaaaaaaaaaaaaaaaaaa

SEQ ID NO: 84 - protein, 560 aa, Homo sapiens
MAAGITQPLLTLASSATTAAIPGVSTVVPSQLPTLLQPVTQLPSQVHPQLLQPAVQSMGIPANLGQAAEVPLSSGDVLYQGFPPRLPPQYPGDSNIAPSSNVASVCIHSTVLSPPMPTEVLATPGYFPTVVQPYVESNLLVPMGGVGGQVQVSQPGGSLAQAPTTSSQQAVLESTQGVSQVAPAEPVAVAQPQATQPTTLASSVDSAHSDVASGMSDGNENVPSSSGRHEGRTTKRHYRKSVRSRSRHEKTSRPKLRILNVSNKGDRVVECQLETHNRKMVTFKFDLDGDNPEEIATIMVNNDFILAIERESFVDQVREIIEKADEMLSEDVSVEPEGDQGLESLQGKDDYGFSGSQKLEGEFKQPIPASSMPQQIGIPTSSLTQVVHSAGRRFIVSPVPESRLRESKVFPSEITDTVAASTAQSPGMNLSHSASSLSLQQAFSELRRAQMTEGPNTAPPNFSHTGPTFPVVPPFLSSIAGVPTTAAATAPVPATSSPPNDISTSVIQSEVTVPTEEGIAGVATSTGVVTSGGFPIPPVFESPVLSSVVSSITIPARQHI

SEQ ID NO: 85 - DNA, 3325 bp, Homo sapiens
cttggctcacaggggacgatgtcaagctcttcctggctccttctcagccttgttgctgtaactgctgctcagtccaccattgaggaacaggccaagacatttttggacaagtttaaccacgaagccgaagacctgttctatcaaagttcacttgcttcttggaattataacaccaatattactgaagagaatgtccaaaacatgaataatgctggggacaaatggtctgcctttttaaaggaacagtccacacttgcccaaatgtatccactacaagaaattcagaatctcacagtcaagcttcagctgcaggctcttcagcaaaatgggtcttcagtgctctcagaagacaagagcaaacggttgaacacaattctaaatacaatgagcaccatctacagtactggaaaagtttgtaacccagataatccacaagaatgcttattacttgaaccaggtttgaatgaaataatggcaaacagtttagactacaatgagaggctctgggcttgggaaagctggagatctgaggtcggcaagcagctgaggccattatatgaagagtatgtggtcttgaaaaatgagatggcaagagcaaatcattatgaggactatggggattattggagaggagactatgaagtaaatggggtagatggctatgactacagccgcggccagttgattgaagatgtggaacatacctttgaagagattaaaccattatatgaacatcttcatgcctatgtgagggcaaagttgatgaatgcctatccttcctatatcagtccaattggatgcctccctgctcatttgcttggtgatatgtggggtagattttggacaaatctgtactctttgacagttccctttggacagaaaccaaacatagatgttactgatgcaatggtggaccaggcctgggatgcacagagaatattcaaggaggccgagaagttctttgtatctgttggtcttcctaatatgactcaaggattctgggaaaattccatgctaacggacccaggaaatgttcagaaagcagtctgccatcccacagcttgggacctggggaagggcgacttcaggatccttatgtgcacaaaggtgacaatggaggacttcctgacagctcatcatgagatggggcatatccagtatgatatggcatatgctgcacaaccttttctgctaagaaatggagctaatgaaggattccatgaagctgttggggaaatcatgtcactttctgcagccacacctaagcatttaaaatccattggtcttctgtcacccgattttcaagaagacaatgaaacagaaataaacttcctgctcaaacaagcactcacgattgttgggactctgccatttacttacatgttagagaagtggaggtggatggtctttaaaggggaaattcccaaagaccagtggatgaaaaagtggtgggagatgaagcgagagatagttggggtggtggaacctgtgccccatgatgaaacatactgtgaccccgcatctctgttccatgtttctaatgattactcattcattcgatattacacaaggaccctttaccaattccagtttcaagaagcactttgtcaagcagctaaacatgaaggccctctgcacaaatgtgacatctcaaactctacagaagctggacagaaactgttcaatatgctgaggcttggaaaatcagaaccctggaccctagcattggaaaatgttgtaggagcaaagaacatgaatgttaggccactgctcaactactttgagcccttatttacctggctgaaagaccagaacaagaattcttttgtgggatggagtaccgactggagtccatatgcagaccaaagcatcaaagtgaggataagcctaaaatcagctcttggagataaagcatatgaatggaacgacaatgaaatgtacctgttccgatcatctgttgcatatgctatgaggcagtactttttaaaagtaaaaaatcagatgattctttttggggaggaggatgtgcgagtggctaatttgaaaccaagaatctcctttaatttctttgtcactgcacctaaaaatgtgtctgatatcattcctagaactgaagttgaaaaggccatcaggatgtcccggagccgtatcaatgatgctttccgtctgaatgacaacagcctagagtttctggggatacagccaacacttggacctcctaaccagccccctgtttccatatggctgattgtttttggagttgtgatgggagtgatagtggttggcattgtcatcctgatcttcactgggatcagagatcggaagaagaaaaataaagcaagaagtggagaaaatccttatgcctccatcgatattagcaaaggagaaaataatccaggattccaaaacactgatgatgttcagacctccttttagaaaaatctatgtttttcctcttgaggtgattttgttgtatgtaaatgttaatttcatggtatagaaaatataagatgataaagatatcattaaatgtcaaaactatgactctgttcagaaaaaaaattgtccaaagacaacatggccaaggagagagcatcttcattgacattgctttcagtatttatttctgtctctggatttgacttctgttctgtttcttaataaggattttgtattagagtatattagggaaagtgtgtatttggtctcacaggctgttcagggataatctaaatgtaaatgtctgttgaatttctgaagttgaaaacaaggatatatcattggagcaagtgttggatcttgtatggaatatggatggatcacttgtaaggacagtgcctgggaactggtgtagctgcaaggattgagaatggcatgcattagctcactttcatttaatccattgtcaaggatgacatgctttcttcacagtaactcagttcaagtactatggtgatttgcctacagtgatgtttggaatcgatcatgctttcttcaaggtgacaggtctaaagagagaagaatccagggaacaggtagaggacattgctttttcacttccaaggtgcttgatcaacatctccctgacaacacaaaactagagccaggggcctccgtgaactcccagagcatgcctgatagaaactcatttctactgttctctaactgtggagtgaatggaaattccaactgtatgttcaccctctgaagtgggtacccagtctcttaaatcttttgwatttgctcacagtgtttgagcagtgctgagcacaaagcagacactcaataaatgctagatttacacactcaaaaaaaaaaaaaaaaa

SEQ ID NO: 86 - protein, 805 aa, Homo sapiens
MSSSSWLLLSLVAVTAAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMEDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSIWLIVFGVVMGVIVVGIVILIFTGIRDRKKKNKARSGENPYASIDISKGENNPGFQNTDDVQTSF

SEQ ID NO: 87 - DNA, 1276 bp, Homo sapiens
gttgcggacatgaatcttgttatttgtgtcctacttttgtccatttggaaaaataattgcatgactacaaaccaaaccaatggatcttctactacaggagataaacctgttgaatcaatgcagacaaaattgaactaccttagaagaaatctactcattttagttggtattatcatcatggtttttgtctttatctgtttttgttatctccattataattgtctgagcgatgatgcgtccaaagcaggaatggtcaagaaaaaaggcatagcagccaagtcatctaaaacatcattcagtgaagccaagacagcctctcaatgcagtccagaaacacaacccatgctatctactgcagacaagtcatctgattcatcgagtccagaaagggcatccgcacaatccagcacagaaaaattaatcagaccctcaagtctacaaaagccatccataccaaacagtgcaggaaagttaactaggccatcatatccaaaaaggtcatccaagtcatcatgctcaaaaaaattaagcaagtcatctcacctggaaaaggcacataagaaaggcagcctagaaaaattatgtaagctagactatgcgtgtaagctagccagttcagataagccagtcaggccacctcagctattcaagccactttattcatctcatccacaaaatgaaatctcaccatccaagccattcggtccacaggaattggctaagcctcccaaacattttaatccaaaaaggtcagtgagtctaggcagggcagccttattatccaactctgaattagctgaaacttgtcaaccctacaagaaaaaamatcttgttgccaaaacttataggcctttggtcaatgatatttctgaggcaaaggagaaaaacactcaaaacctacatgtttcaagcaaagtcaagtcytcttccaggtcctttcgtaagttagattccaggaacaatgcatacggtgatcatgtgaatgacagtgatacgatgaaatattatagtgaggttgacagtgataaagttataatcattacgtgtggcagagggtacaatcaagtcacctctgaagtaaccctaaatgattaggagctcaacaaaaataaaatccaagtgtgaaagagcaaacttataccaactattttatgctcatcatcccttaagaaaccagtagagggaaaaatcactatcaagatcacccatatctgtcttggaggcgatttattttaaaaatatcaataaaatgcatgacataaaaaaaaaaaaaaaa

SEQ ID NO: 88 - protein, 361 aa, Homo sapiens
  UNSURE (273)
MNLVICVLLLSIWKNNCMTTNQTNGSSTTGDKPVESMQTKLNYLRRNLLILVGIIIMVFVFICFCYLHYNCLSDDASKAGMVKKKGIAAKSSKTSFSEAKTASQCSPETQPMLSTADKSSDSSSPERASAQSSTEKLIRPSSLQKPSIPNSAGKLTRPSYPKRSSKSSCSKKLSKSSHLEKAHKKGSLEKLCKLDYACKLASSDKPVRPPQLFKPLYSSHPQNEISPSKPFGPQELAKPPKHFNPKRSVSLGRAALLSNSELAETCQPYKKKXLVAKTYRPLVNDISEAKEKNTQNLHVSSKVKSSSRSFRKLDSRNNAYGDHVNDSDTMKYYSEVDSDKVIIITCGRGYNQVTSEVTLND

SEQ ID NO: 89 - DNA, 997 bp, Homo sapiens
  unsure (96)
  unsure (100)..(101)
  unsure (104)
  unsure (110)
  unsure (114)
  unsure (206)
  unsure (209)
  unsure (211)..(212)
  unsure (269)
  unsure (272)
  unsure (723)
tagctttctgaaacaaaatccacaaaatattggtgaccatatgttgacctgctcattatctccaaagatagacttaccagaggtgcaaattgagcntgannctnaattanaaanagaaagccctggcttgaaaaacagtccaattgatgaaagtgaggtgcaaacagcaactgatagtccctctgttaaacctaatgagcttgaanaananntttctcccagcattcaaacagaaactttggtacagcaggaagagccttgtgaggaanaanctgaaaaagcaacatgtgattctgactttgctgttgaaactttggagcttgaaactcmaggagaggaggtcaaagaaraaattcytcttgtagcatccgcttcagtcagtattgaacaattcactgaaaatgccgargagtgtgctttaaatcagcagatgtttaacagtgacttggagaagaaaggggcagaaattattaaccctaaaacagcattgttaccatctgacagtgtgtttgcagaagaaaggaacctcaaaggaattctagaagaatctccatctgaagcagaagatttcatttctggaattacacagactatggtagaagctgtagctgaagtagaaaaaaatgaaactgtttcggaaatattgccatcaacttgtattgtgacgttagtaccaggaattcccactggggatgagaagacagtggacaaaaagaatatttnggaaaaaaaaggtaacatggatgaaaaggaggagaaggaatttaatactaaggaaaccagaatggatcttcaaataggaacagagaaggctgaaaagaatgaaggtaggatggatgcagaaaaggtggaaaagatggcagcaatgaaagaaaagcctgcagaaaacactttattttcaaggcatacccaaataaaggagtgggtcaggctaataagcctgatgaaactagtaaaactagtatttctggctgtatcagatgtatctagcagtaaa

SEQ ID NO: 90 - protein, 319 aa, Homo sapiens
  UNSURE (19)..(22)
  UNSURE (24)..(25)
  UNSURE (56)..(58)
  UNSURE (77)..(78)
  UNSURE (97)
  UNSURE (104)
  UNSURE (106)
  UNSURE (228)
MLTCSLSPKIDLPEVQIEXXXXLXXESPGLKNSPIDESEVQTATDSPSVKPNELEXXXSPSIQTETLVQQEEPCEEXXEKATCDSDFAVETLELETXGEEVKEXIXLVASASVSIEQFTENAEECALNQQMFNSDLEKKGAEIINPKTALLPSDSVFAEERNLKGILEESPSEAEDFISGITQTMVEAVAEVEKNETVSEILPSTCIVTLVPGIPTGDEKTVDKKNIXEKKGNMDEKEEKEFNTKETRMDLQIGTEKAEKNEGRMDAEKVEKMAAMKEKPAENTLFSRHTQIKEWVRLISLMKLVKLVFLAVSDVSSSK

SEQ ID NO: 91 - DNA, 425 bp, Homo sapiens
  unsure (29)
  unsure (37)
  unsure (48)
  unsure (67)
  unsure (71)..(72)
  unsure (150)
  unsure (269)
  unsure (348)
  unsure (363)
  unsure (422)
attcttctgtccaatttgttccttcttcnactcaggngaaaaagcaangacaaatcactgcaagagnacanntcataagcaaaatactgagaaattcatggccaagcaaagaaaggaaaaggagcagaatgaggctgaagaaagaagttntaggtgattgggggaaaggaaagaattcactagaaatttgtttagggtccagttgatttgtgtatttttgttatcatttaatttgtaattttcgtttcagaagcaaatattcgtgttgnacaaatttctgattgccctaaatgtagagagactgatggggaaagtatgatgggtttgatttttatatcaaatcatcangcatggagaaatatnttttagaagtgttaaaataaatgttcctactgtatatttaaaatacaaaaaaaaaaaanaaa

SEQ ID NO: 92 - DNA, 2723 bp, Homo sapiens
cagatggagaggccaggcttattgattccagtgcctactcactccaccatcacgaaggtcatggtgcagcagctagccgccgagggacttccttgacaggcagattccagccttggcaatgccaggaggcaccttctctcttcaatctcgggacacctcatgtggggagaccaagggccacaggaccgtgagagggctcagcccagctttgttctggacagacggagaggcggctcctgatccctggcactgccagcagccctgtgaatgagccactgtgttgacccctctggactgcctgcagagtgtgtgccccagtcacaccagatcaatttcactccttgaacatgccagaatgggtgcttagaagtgaaatcactgggtcaaaagagtaattaatcatcatggaagaatatcgtggaaatgtagaggtgcaaaataaattaaataaagttggcaaaataaatatacaaatgactggtgatatgatctgaatgttgatatctcctcaaaatttatttatgttgaaacctagtacccaatatgatggtattagagatggagttttagggaagtggttaagtcatgagggctccaagctcgggaatggcatcagcgtccttacaaacgaagcttcagggagcccccttgccccttcagccacatgaagacacagaaggtgccgtctatttggaacgggccctcaccagacaccgaatcagctggcaccttgatcttgaagttcctatcctccagaactgtcaataatacattttctgttgtttataaatttcccactctaaggtattttttaatagcagtccaaaggaaataagacaactgaataatgaatggataatcctgtcacccagtgataactcattttagttgtcatgctaaaatgctcactaccttctaataattttaaccatgcttattgtttttaaaatatagattgtgatttacatacaattttcatcatgcatctgtgataaatactataccacaagtaattctctacattattaaaaatatattcattattaaaaacatcttaaacttttcagttgtatgaatgaactgttactattcaaagaattcctctaaggggtaatcatttagcacgttttcacttttgcctcctataaataaaaatttgttgagcattttttatcagctatattaaggtaaaatttacgtactgtagaaatcacctggatgaacacatttttatgagcttctatcttgcgtgtttcagtgtttcctaaggctagatatcacaaattaaccaagtaagacaaagggcctgatacatgtttaggttcaagacaaactttgccaaatgccttctactattgatttatactttcgtcagcagagtattagagtgctcatctcattccacccaggtcagaatgaatagtattattattcaagatgctttcacttttagggaaaaagtcaagacatcaacatatgtgttcaaagaaatggaaagtgcatttgggaattttgtttttcactttcgatgcactttgagtgtttttgtcattttcttccttcttgtttagttgaggtcatcttcttgtcagcttcttgtttagttggggtaatatgctgttcaaatatattgcccttattgaacctgttataataaccaaggtagaaagtggcaatatgagtaactttcctgcaactcaaacagcagcctcacctaaaccaagaaacctccaactctgggttcctagtcacgaactacattaccctagacaaggatttaaacttctctgagcattatttttctcatagcaaaaaggaaaaagaaatagaagatgatcaagttctaggcacttaaccagcgataccaaaaaatttaataacattctatatttgaataatagaaaacgatttttaaaatattattctaccctctaattcaaatcgcccttgtgaaaacttgctcttaaaactatgaaagaatggtaaaataactaattatgtgtgacttactctcatgacttatcccattcttctaagtcttgcagtgcttaaaaaaaaataaaggatgtgtaactgctagttatcagcactttatataacttttgattcaagtaacaaatagcattccagttctatgtattaattatgccactaaattgcaaaggaaagaagccaattctaatgaattgtaaccatagtttcttttgcgtgttacatattagaagtgggtagcactttaaattaccatacacaaaacagcatataattaattctgaataaaagataatgtattcttgctaaatacgagttattttaaaatctcttaatgaagaactaagcatatgctgtgttgcactgtgaaaatgtttagcatatgccattagagccaacattttgcatttattattgtatttattttcaataataatatattatttaagtgaacatacaaaaaatttaatcactaacattccgaatgtggaaaatatatgcaaattacaggaaactttttggctccccctaagttgtcactaaataactttttgatgacatggtgtgattgcaatttgtaataaaatgagaaaaatggaaaagattgttacttttaagtggcagaacatatgcacaatgcattttaaaattcagatacggaaataaacatttctattctgaaaactgaaaaaaaaaaaaagaaaaaaaaaaaaaaaaaaaaaaaaaaaaaa

SEQ ID NO: 93 - protein, 85 aa, Homo sapiens
MFRFKTNFAKCLLLLIYTFVSRVLECSSHSTQVRMNSIIIQDAFTFREKVKTSTYVFKEMESAFGNFVFHFRCTLSVFVIFFLLV

SEQ ID NO: 94 - DNA, 176 bp, Homo sapiens
  unsure (90)
  unsure (124)
  unsure (164)
cgacagagggttggaggaagccgccgggttgctggcccgtgcctcggtggcctggctcgggccgaaaacggatcttggcgggttgccccnctgctctcaggctcgctgtggtctggcttcctgnagcaagcctctgtttgctcatctctacgttggggatgacngtgggatggggc

SEQ ID NO: 95 - DNA, 721 bp, Homo sapiens
  unsure (53)
  unsure (117)
  unsure (137)
  unsure (156)
  unsure (174)
  unsure (472)
  unsure (475)
  unsure (480)
  unsure (505)
  unsure (534)
  unsure (606)
  unsure (697)
  unsure (705)..(706)
cactgttgcatcctgactatatttcatgttcagagcacagggaaggtcacagntttgaatgtcagaatgtcagacttgaatcatgttaaagtcctgcctctgactcctgactgctgngcagccttggacaagttaanacaccttcctgagcttcantttccctttgtaaagggngaaataataacgacctttcatacagggttgctgggatgatcagtgattttgctaatatcaaaagtgcccagcacagtgcttgggttgttggarrmawwgaacacacggcattgttattatttatatgccttgtaactggaagagcctgtgggcaaacagtggatgctaaaattcagtttgtggaagaaccaggtgcacaaactcctgttctacctgtggttgagtctacactcccccaccacaccccagctgctctgatctacctcctgttccttgagcaggccattttctttcttgnttnagggngtttgcaatggctgttgcctggaanacccacctcctgcctttctcaccactgantctttctcatcctttgggccccatgtccaatgtcatctttgcccgtgggagccctgcttgggttcctgagtntcttgtaaaatctcaaacatcttaggaagagtttagggtttgttagtcattcacatcttggtgtgaattcgtcagagcaggtctagaattnaatcgggnnggccatgaaggccgc

SEQ ID NO: 96 - protein, 107 aa, Homo sapiens
  UNSURE (20)..(21)
  UNSURE (88)..(89)
  UNSURE (91)
  UNSURE (99)
MISDFANIKSAQHSAWVVGXXEHTALLLFICLVTGRACGQTVDAKIQFVEEPGAQTPVLPVVESTLPHHTPAALIYLLFLEQAIFFLXXGXLQWLLPGXPTSCLSHH

SEQ ID NO: 97 - DNA, 433 bp, Homo sapiens
gtcgaccttgaagatgttttctaaagaatcaaaaatgctacaaagaagtatatcattttcaaatatggctttatcgtcttgtttacttttaccaggagatgccactgtcataacttcttcatgggataataatgtctatttttattccatagcatttggaagacgccaggacacgttaatgggacatgatgatgctgttagtaagatctgttggcatgacaacaggctatattctgcatcgtgggactctacagtgaaggtgtggtctggtgttcctgcagagatgccaggcaccaaaagacaccactttgacttgctggccgagctggaacatgatgtcagtgtagatacaatcagtttaaatgctgcaagcacactgttagtttccggcaccaaagaaggcacagtgaatatttgggacctcacaacggcc

SEQ ID NO: 98 - protein, 140 aa, Homo sapiens
MFSKESKMLQRSISFSNMALSSCLLLPGDATVITSSWDNNVYFYSIAFGRRQDTLMGHDDAVSKICWHDNRLYSASWDSTVKVWSGVPAEMPGTKRHHFDLLAELEHDVSVDTISLNAASTLLVSGTKEGTVNIWDLTTA

SEQ ID NO: 99 - DNA, 84 bp, Homo sapiens
  unsure (8)
tttttttnaaaaaaaaaaaaaaaaaaaaaaaaaaaaaaaaaaaaaaaaaaaaaaaaaaaaaaaaaaaaaaaaaaaaaaaaaaaa

SEQ ID NO: 100 - DNA, 254 bp, Homo sapiens
  unsure (148)
aagctttccctgccaggacacagcactgttttctaaagggaacagatattagagatgaggacagtatggttggatttgaatatgattgcttagattgaataatttctccaattggagtctgtttttctctaagttttaaattgagganaaaacagatgagccagacatggaaatttctaattttaaattcaaattttgaatattttgatttagtaatttgagaacaagcaatttgtaattctagtgagccttac

SEQ ID NO: 101 - DNA, 733 bp, Homo sapiens
gtcgaccttgaagatgttttctaaagaatcaaaaatgctacaaagaagtatatcattttcaaatatggctttatcgtcttgtttacttttaccaggagatgccactgtcataacttcttcatgggataataatgtctatttttattccatagcatttggaagacgccaggacacgttaatgggacatgatgatgctgttagtaagatctgttggcatgacaacaggctatattctgcatcgtgggactctacagtgaaggtgtggtctggtgttcctgcagagatgccaggcaccaaaagacaccactttgacttgctggccgagctggaacatgatgtcagtgtagatacaatcagtttaaatgctgcaagcacactgttagtttccggcaccaaagaaggcacagtgaatatttgggacctcacaacggccaccttaatgcaccagattccatgccattcagggattgtatgtgacactgcttttagcccagatagtcgccatgtcctcagcacaggaacagatggctgtcttaatgtcattgatgtgcagacaggaatgctcatctcctccatgacatcagatgagccccagaggtgctttgtctgggatggaaattccgttttatctggcagtcagtctggtgaactgctcgtttgggacctccttggagcaaaaatcagtgagagaatacagggccacacaggtgctgtgacatgtatatggatgaat

SEQ ID NO: 102 - protein, 240 aa, Homo sapiens
MFSKESKMLQRSISFSNMALSSCLLLPGDATVITSSWDNNVYFYSIAFGRRQDTLMGHDDAVSKICWHDNRLYSASWDSTVKVWSGVPAEMPGTKRHHFDLLAELEHDVSVDTISLNAASTLLVSGTKEGTVNIWDLTTATLMHQIPCHSGIVCDTAFSPDSRHVLSTGTDGCLNVIDVQTGMLISSMTSDEPQRCFVWDGNSVLSGSQSGELLVWDLLGAKISERIQGHTGAVTCIWMN

SEQ ID NO: 103 - DNA, 296 bp, Homo sapiens
  unsure (52)
  unsure (62)
  unsure (92)
  unsure (116)
  unsure (160)
acttgtcagaaagcactgaacaattaagaaattttccaagaaaatgtgcagnattctctgcnacttcagaatctgttttgtcttcctaatcnatcacaattgccacccatcgggtnttgggtgtgtgttttcatagcgtggttactttctataatgctgnacccagattctaagaacctggagaaggattagcagttcttagtaagtttactgtgtataggaacggtttgtatttcattacagctattcatcttttctacattaaaaatatttttctctaaaaaaaaaaaaaaaaa

SEQ ID NO: 104 - DNA, 910 bp, Homo sapiens
  unsure (799)
  unsure (856)
  unsure (863)
tgtgtgcaaagtacagaagcagaggacaatgagttctgtcttctcggctccacagattccaaggaagaacagaggcagtgacaagactcaccgaggacctcctgcccaccagaccctgccgagaggctgccatgatggctcaccagaggccgagatgaccctaaggctaaccccactttaagatgaggagcctgaggctcgtgttagtttggttcctggcatgacgctgtggcagggctggggagctcatctctctgacccagagctcacggggatgagctccagttggaatgaatgagtcaggtactttttgatagttggaagttaacgctttatcactgcattaccgattgtttaggagctcaggcccttccaggtggagcaccctctaagcaaagtgtaaagtgttctgaaccacagtggcgccacttcatggaggatcgattgtatttgagggttggagkgtgagaacagcaaaaaataataattcaaccagttggttattatgaacatcattttcatatttttaaaaatatgctatatcatggaattcaatgtaaaacctcaagagatgccatccttggagagggctgcaccagcctgtgccccaagttacccaggatcaccccctacttctcctgacggccccccgagaaaggcctgcattctgggcgacgtggccttcaggggctcaacccttggcctcgagrgggggcccggtacccaattcgccctatagtgagtcgtattacaattcactggccgtcgttttacaacgtcgtgactgggaaaaccctgncgttacccaacttaatcgccttgcagcacatccccctttcgccagctggcgtaatancgaagangcccgcaccgatcgcccttcccaacagttgcgcagcctgaatggcga

SEQ ID NO: 105 - protein, 77 aa, Homo sapiens
  UNSURE (69)
MNIIFIFLKICYIMEFNVKPQEMPSLERAAPACAPSYPGSPPTSPDGPPRKACILGDVAFRGSTLGLEXGPGTQFAL

SEQ ID NO: 106 - DNA, 1032 bp, Homo sapiens
cttcgttcaagtgtgagctgcggctgagcccagcgctcgaggcgcgaggcagccaggagggcccgtgcggcgcggggagccagcgagcgcgccttcggcattggccgccgcgatgtcagctcagtgctgtgcgggccacctggcctgctgctgtgggtctgcaggctgctctctctgctgtgattgctgccccaggattcggcagtccctcagcacccgcttcatgtacgccctctacttcattctggtcgtcgtcctctgctgcatcatgatgtcaacaaccgtggctcacaagatgaaagagcacattcctttttttgaagatatgtgtaaaggcattaaagctggtgacacctgtgagaagctggtgggatattctgccgtgtatagagtctgttttggaatggcttgtttcttctttatcttctgtctactgaccttgaaaatcaacaacagcaaaagttgtagagctcatattcacaatggcttttggttctttaaacttctgctgttgggggccatgtgctcaggagctttcttcattccagatcaggacacctttctgaacgcctggcgctatgtgggagccgtcggaggcttcctcttcattggcatccagtcctcctgctcgtggagtttgcacataagtggaacaagaactggtgtgtgcctttatggaaagcttcccattgactcacagaaactgcccagttttgaccaaggctgtactcaactgcattgctagggatttgcagttttgtttccctttatacctgcttttttgtacctcttcatatactcctctccttcattcacttctcactttttgaccccctgcccctactcccttgcttgggctctgagtcaaccagtggtgtgaattagccacactcaatcccctgctcgtacgggtctcgatctcctgacctcgtgatccgcccacctcggcctcccaaagtgctgggattacaggctcgagccaccgcacctggcctgatgdttctgcaaaaaaaaaaaaaaaaaaaaaaaaaa

SEQ ID NO: 107 - protein, 210 aa, Homo sapiens
MSAQCCAGHLACCCGSAGCSLCCDCCPRIRQSLSTRFMYALYFILVVVLCCIMMSTTVAHKMKEHIPFFEDMCKGIKAGDTCEKLVGYSAVYRVCFGMACFFFIFCLLTLKINNSKSCRAHIHNGFWFFKLLLLGAMCSGAFFIPDQDTFLNAWRYVGAVGGFLFIGIQSSCSWSLHISGTRTGVCLYGKLPIDSQKLPSFDQGCTQLHC

SEQ ID NO: 108 - DNA, 1626 bp, Homo sapiens
  unsure (21)
  unsure (96)
agggccaggttttccgggccntcacattgccaaaagacggcaatatggtgggaaataacatatagacaaacgcacaccggccttattccaagcggnttcggccagtaacgttagaattgcggccgcaggtytaggtcagagccaaaggaaagcttgaaaaatgaagacattagcaggacttgttctgggacttgtcatctttgatgctgctgtgactgccccaactctagagtccatcaactatgactcagaaacctatgatgccaccttagaagacctggataatttgtacaactatgaaaacatacctgttgataaagttgagattgaaatagccacagtgatgccttcagggaacagagagctcctcactccacccccacagcctgagaaggcccaggaagaggaagaggaggaggaatctactcccaggctgattgatggctcttctccccaggagcctgaattcacaggggttctggggccacacacaaatgaagactttccaacctgtcttttgtgtacttgtataagtaccaccgtgtactgtgatgaccatgaacttgatgctattcctccgctgccaaagaacaccgcttatttctattcccgctttaacagaattaaaaagatcaacaaaaatgactttgcaagcctaagtgatttaaaaaggattgatctgacatcaaatttaatatctgagattgatgaagatgcattccgaaaactgcctcaacttcgagagcttgtcctgcgtgacaacaaaataaggcagctcccagaattgccaaccactttgacatttattgatattagcaacaatagacttggaaggaaagggataaagcaagaagcatttaaagacatgtatgatctccatcatctgtacctcactgataacaacttggaccacatccctctgccactcccagaaaatctacgagcccttcacctccagaataacaacattctggaaatgcacgaagatacgttctgcaatgttaaaaatttgacttatattcgtaaggcactagaggacattcgattggatggaaaccctattaatctcagcaaaactcctcaagcatacatgtgtctacctcgtctgcctgttgggagccttgtctaatttcagataatggttagcattacgatggctactataaataaaccattcttactgctctcttccaaaacaaaactcagcatgatactttgagattgtgttctgagagatgatatgactacataaaatacaattaaaaatgttataatataatgaaaatgtagtaatttaagaaaacaccagatgagttaggaataaacctataacatttacaaaaagagcaaaaytaagtgatagaaaatatttcacacatgttcttatagatcatgtatcacttgcaagttttaggagttcatatcctatatcatttcaaattaagtacataataaagtaaaattttgaaatgaacactttaggtatttttgccaagatttagatgtttttaattaaacttttytcttcctttttttttcactaaagcatgtttattcccctaatccattaaagagcatgaaaaaaagaataaatgtatttgaaaattaaaaaaaaaaaaaaaaaaa

SEQ ID NO: 109 - protein, 322 aa, Homo sapiens
MKTLAGLVLGLVIFDAAVTAPTLESINYDSETYDATLEDLDNLYNYENIPVDKVEIEIATVMPSGNRELLTPPPQPEKAQEEEEEEESTPRLIDGSSPQEPEFTGVLGPHTNEDFPTCLLCTCISTTVYCDDHELDAIPPLPKNTAYFYSRFNRIKKINKNDFASLSDLKRIDLTSNLISEIDEDAFRKLPQLRELVLRDNKIRQLPELPTTLTFIDISNNRLGRKGIKQEAFKDMYDLHHLYLTDNNLDHIPLPLPENLRALHLQNNNILEMHEDTFCNVKNLTYIRKALEDIRLDGNPINLSKTPQAYMCLPRLPVGSLV

SEQ ID NO: 110 - DNA, 1083 bp, Homo sapiens
  unsure (1033)
  unsure (1035)
  unsure (1039)
  unsure (1053)
  unsure (1058)
  unsure (1062)..(1066)
  unsure (1074)
cwaaaacatgagaccwrggcwcagaccttactgtatgagaagcaatgctcctcaaaccttctgcgtgctgacatagaccttcccaraagcwaaactgttggcggcgacctgagcgctggaagccgaaggggaagaggagggagacgcgaagccagggcggycggcacwwaggcggcggactcgcggsggcagcgcctgcccggccgggagcacmacccacggccctactccagcgaagtcccgcwccggcttctaggrataaagtttacgttytcctgaggccgcaccccccacytcccacccaggacggcacatctccgtgtcytcctcccccaaaytccaytmgggaccccgagaaccaccccagcyttccggccaccacaacaaagagccgcwccgaccggcgaggatwaacagcggcggagggcgakagggcggcggggcgagcgcctccacgcagcaactccggagtcccccgcttgcccgagcgcagtttctccgctgctgtttccaccggctttgtaacactgggaatttacatcctcacccgcacccctcacgcccgaggattttaaactcacctttactctcgaactgagagttgcggtagatgggatttttgccttttccccagatggttgaaggttaagatttttggaaaccccmccacctccttatttctattattatttctgcaagaaaagtataaagagagttgtagtggaggtgagatttgtgatcgggaaagccttcgactccctccttctccgtcttccgcytctctctctctgattagttcctatccagcagcagattgaagcaggagatgattcttctcaaggtttgttcagcagcttcacttctaggcgaaggcttcatgaaccaagtgacgtcaaccaacaaggcttctctctctctcctctctctaacaatgaaagttgctgttaacaagggaaaaaaagagagagaattgtttataccatttcagttccaataataaakgacctatcagytcctaaaggagccaaaaaananaaanaaaaaaaaaaaaanaaganaagnnnnnaaaaaaanaaaaaaaaa

SEQ ID NO: 111 - protein, 56 aa, Homo sapiens [sequence omitted]

SEQ ID NO: 112 - DNA, 643 bp, Homo sapiens
gaaagaggagctgggcggggtggggggaaggcggaggcagtytagtaatgtaaagctccgctgagagggagagtgccgccctaaacactcatgctgccagtccccaaaagacttcattcattcaacatatatgtgaccgcctgctacgtgccaggcgtgggccaggtcctagggacaaaggagaggcctccgcaccccaccccatgacccatacctcctcttccccacctccctgggccagcctgccttccttctccctcctcctccttcctgggggaaggaagccccaccttctgtgcgcagtcagctcctaagcacgctcccgcttcccctggcctccccatttaaaaagggaggcaaaggatgtcaccactgtcactacactcatggctttgctctgggaagtcctgcaaataaaatgaaagttctccaacccctccmtacccaytcgggccacaaaggcgaggggaggcaggtytgaggcagaggagccagggcaggtgcggcgcttccgcytctggtcccaaagcaaagaktccccctgtgactgacagcccgtgttatgttaaatacattttgttggtttgtaattcaaatcccataaagcaggaggtagagagccaaaaaaaaaaaaaaaaaaaaa

SEQ ID NO: 113 - protein, 38 aa, Homo sapiens
MLPVPKRLHSFNIYVTACYVPGVGQVLGTKERPPHPTP

SEQ ID NO: 114 - DNA, 1047 bp, Homo sapiens
aggcgatgccccataaaagggctcctgaagctctttgtgaaggggtctgaaaagggcagatgagggtgtcctttggggcggatgtggggtttgggacaattgcatgtgattgtcattctttagctgtctgcatcccacagaaacttttttctgagtcttccagctggcccaagtcctgggtctcttttactgttcttgtagctgactacagtaggcagatgaggaactcttagtcaatctgggaaaactcgactgactataaacaatcctaaattgaaagaagtgtatggcattggggggtgggtgtgatagtacagagtggagcctgcaagttcacgcaccgggaaccaaaccccacctaacttggactggacgtcctcttccagggaatccagccagggccaattagaaatgtgtcttaaattggtgtcaggtcaccaaaaacaaaaacaggatccatgggggcctgtgaaacttcgagtgccattcatgctcggttcagtcatctgactcgtcaggatgaccagcagtgctccctgtaactcgccattatctgaccaattactggagctactttataatgaggcttctggagctactttataatgaggcttctgtttgctgtcatggtggggagtttggaattgtggcttcttgcctaacaccaatgagaggactttgggacaaacccccagagccaggagtgttttgaggcctagtggggttgggaacaaagggtcaagtgtcgagggagtggggaaattatgggttggggacaggtgtgaacagtgggcttgggggtggcccaagagtactagaccagagaggtccagtgccacccgcagcctgcagtgatactagacagggggcggctgtgtggaaccacagatgacatcccttctcctcttgatggaagtggaggctgcatctgagagcttcccagcctagattctggtggctgcatctgagagcttcccagcctagattctggtgggattggatctgggagggggaagaccccaaaaagcaaaaaaaaaaaaaaaaaaa

SEQ ID NO: 115 - protein, 57 aa, Homo sapiens
MRVSFGADVGFGTIACDCHSLAVCIPQKLFSESSSWPKSWVSFTVLVADYSRQMRNS

SEQ ID NO: 116 - DNA, 2851 bp, Homo sapiens
atttcgtacagtaggagatttcaacaacgtgacaatattctctaggcacttgggctcactgtctgtagcccccaccccccgcctttcgccacctccttgcttccctactcccccttctgcttttgcctttgatgagtttttggcttactttttggcggagtctcttggacacgtttttgctggtgctggaagatcagatacatggaacctttgaaaactgattatttttctccgatatgacttaaaaaaaaataaaaagaagaaaagaaaatagagtagtgcacggcaagctagaggattgtaaattttccttggtgaactttgaggatccataaagaagaaatggttctctttactgcgaggctgcaaggtcacccaatgagagaggggccaaataagctggaacatcatctaatacactgaatgtagccactctgtgtcttttgattggagagtttagtccatttacattcaatgctataattggagttactggaaaagcaagaataacttatgcggattaacaatatggaaacatcctgagactactttggaatcgccataaattaagtgggttccagttttgcaaacagagaaacggtccatgaacaatttgctacaggtataaagaagtatctgcagaaatccagagcacttattaaacttctttgagttttctcaggaagatcaatacaagatggagaaattttattaagattggcaaacgcactgcctacttacagcatagagacccccagtggagagctagactgtttgaattccagaaggaccaacaccagataaattatgaatgttgaacaagatgaccttacatccacagcagataatgataggtcctaggtttaacagggccctatttgaccccctgcttgtggtgctgctggctcttcaacttcttgtggtggctggtctggtgcgggctcagacctgcccttctgtgtgctcctgcagcaaccagttcagcaaggtgatttgtgttcggaaaaacctgcgtgaggttccggatggcatctccaccaacacacggctgctgaacctccatgagaaccaaatccagatcatcaaagtgaacagcttcaagcacttgagacacttggaaatcctacagttgagtaggaaccatatcagaaccattgaaattggggctttcaatggtctggcgaacctcaacactctggaactctttgacaatcgtcttactaccatcccgaatggagcttttgtatacttgtctaaactgaaggagctctggttgcgaaacaaccccattgaaagcatcccttcttatgcttttaacagaattccttctttgcgccgactagacttaggggaattgaaaagactttcatacatctcagaaggtgcctttgaaggtctgtccaacttgaggtatttgaaccttgccatgtgcaaccttcgggaaatccctaacctcacaccgctcataaaactagatgagctggatctttctgggaatcatttatctgccatcaggcctggctctttccagggtttgatgcaccttcaaaaactgtggatgatacagtcccagattcaagtgattgaacggaatgcctttgacaaccttcagtcactagtggagatcaacctggcacacaataatctaacattactgcctcatgacctcttcactcccttgcatcatctagagcggatacatttacatcacaacccttggaactgtaactgtgacatactgtggctcagctggtggataaaagacatggccccctcgaacacagcttgttgtgcccggtgtaacactcctcccaatctaaaggggaggtacattggagagctcgaccagaattacttcacatgctatgctccggtgattgtggagccccctgcagacctcaatgtcactgaaggcatggcagctgagctgaaatgtcgggcctccacatccctgacatctgtatcttggattactccaaatggaacagtcatgacacatggggcgtacaaagtgcggatagctgtgctcagtgatggtacgttaaatttcacaaatgtaactgtgcaagatacaggcatgtacacatgtatggtgagtaattccgttgggaatactactgcttcagccaccctgaatgttactgcagcaaccactactcctttctcttacttttcaaccgtcacagtagagactatggaaccgtctcaggatgaggcacggaccacagataacaatgtgggtcccactccagtggtcgactgggagaccaccaatgtgaccacctctctcacaccacagagcacaaggtcgacagagaaaaccttcaccatcccagtgactgatataaacagtgggatcccaggaattgatgaggtcatgaagactaccaaaatcatcattgggtgttttgtggccatcacactcatggctgcagtgatgctggtcattttctacaagatgaggaagcagcaccatcggcaaaaccatcacgccccaacaaggactgttgaaattattaatgtggatgatgagattacgggagacacacccatggaaagccacctgcccatgcctgctatcgagcatgagcacctaaatcactataactcatacaaatctcccttcaaccacacaacaacagttaacacaataaattcaatacacagttcagtgcatgaaccgttattgatccgaatgaactctaaagacaatgtacaagagactcaaatctaaaacatttacagagttacaaaaaacaaacaatcaaaaaaaaagacagtttattaaaaatgacacaaatgactgggctaaatctactgtttcaaaaaagtgtctttacaaaaaaaaaaa

SEQ ID NO: 117 - protein, 640 aa, Homo sapiens
MLNKMTLHPQQIMIGPRFNRALFDPLLVVLLALQLLVVAGLVRAQTCPSVCSCSNQFSKVICVRKNLREVPDGISTNTRLLNLHENQIQIIKVNSFKHLRHLEILQLSRNHIRTIEIGAFNGLANLNTLELFDNRLTTIPNGAFVYLSKLKELWLRNNPIESIPSYAFNRIPSLRRLDLGELKRLSYISEGAFEGLSNLRYLNLAMCNLREIPNLTPLIKLDELDLSGNHLSAIRPGSFQGLMHLQKLWMIQSQIQVIERNAFDNLQSLVEINLAHNNLTLLPHDLFTPLHHLERIHLHHNPWNCNCDILWLSWWIKDMAPSNTACCARCNTPPNLKGRYIGELDQNYFTCYAPVIVEPPADLNVTEGMAAELKCRASTSLTSVSWITPNGTVMTHGAYKVRIAVLSDGTLNFTNVTVQDTGMYTCMVSNSVGNTTASATLNVTAATTTPFSYFSTVTVETMEPSQDEARTTDNNVGPTPVVDWETTNVTTSLTPQSTRSTEKTFTIPVTDINSGIPGIDEVMKTTKIIIGCFVAITLMAAVMLVIFYKMRKQHHRQNHHAPTRTVEIINVDDEITGDTPMESHLPMPAIEHEHLNHYNSYKSPFNHTTTVNTINSIHSSVHEPLLIRMNSKDNVQETQI

SEQ ID NO: 118 - DNA, 4531 bp, Homo sapiens
ttgctcttcaactttcttttgaggctttaaattaggcaaatatatttttcttagaatttctgaaaactccttttcactggagcttattttaattatatggatgcattatcctcttaaacttctgtgaaaacaataattggagttttcatttgcattggctgtttcatgagaggtcaagctttttgcttatttattttgatgtttccttttcatgatatctgtgtcttgggatttggttgtatattcacatttttaattagctacttgatttaaataaagtatcagtagttactttcagggttcttggtgattataataattactcacctaccaggcttcttccttgcatgcagctcactacaggaatctgttgtgcctagaagatttcgggacaagtggagaaaccccactccgttaactgcatacacagggaaggagctatgagggagacagaataatttggtctctactattaccttaagagaccttcccctgtcttcgatttaaaaaaaaactactgcatacaaagggtaggagctacaagggacatagaataatttggactccattactaccttaagagaccttcccctgacttctcttttaaaaaacctatgagtctctgtacccctgaacttactttccacacctatttctctcttcacccccaaaatccatattagaatgcccctgcaggctataaagcctttcataaaagtaaaatacccagtcttttcaagagaacaataaaataggcagtctcctacctcttgtcttactctaatataaactccatgaagataagtattgtatccatactgttcatgctgcacagcagttgcccttatctgcagggcgacgcatcccaagacccccagtggatgcttgaaactgcagagagtaacacacgtgattgccaccatcggaacacatttctgttcacgtcttccacccacagatttaatgccttttccatcttaactaagcactcatcatggactgtggccataacttttgcagttttagatgcaacagcaaaactaacattaattttttcttcttcttcacaatttcatgggtagatttgttcttaccgtagatcttagcaacctcagcatatgatgttttttcttttgagaactttcaccttttctcttaaagaaatcactttacagcttctctttggcatatctcaactgccagcatcactgctcttgaactttggggccattattaagtcaaaagagggttacttcaaaacaagcactgagataccaccagagtccatctgataactaagatggtaactacatgactaacaggccggtgacgtataaagcatggatatgctggacaaaggggtgagtcatatcccaggtaggatgaagcagggtgacttgagatttcactattcagtatggtgcacaatttaaaacttaggaattgtttatttcttggaacttttcgtttaatgtttttggactgcagttagccacaggtaactgaaactgtggaaagtgaggattggaggataagcagagactgtggtattcatttcattgcacagtgcctaaaatacagtaggtgtactataaatattttgtaaagggacaacttttctgaaactaaaaatatttatgttttacccaataatttttcttctggaaatttatgctaaggaaatattcagagatgcttacaaagttttatgcatgagtgtccatgttatttgtaattgtgaaaaatgaaaataactcaaaagtttaacagtggtcatctaaagtattgtatacactgtatatataggttgaatagaaggtcatcctattcatttattaatgagaggtacaatctctagggatctgtaaaatctattttgtcttaaccaaagaacaaatttttgacatatcttgaataggatgactataaattatgacttttaaattgttgtaatttttgtactattatctgatatttttatttttatgtattttcgtaagtagtttagagatagtcacattttaaaaatctaagatcaagcaaatgaagcttatttttatgtattcatagtataaaagaccttcagtaaataggtaatatttttgttttattctagaaaacagctccttgaacacagtgagctggcttttcacacattgcagttgttagtgtttactgcccttgccattttaattatgaggctaaagatgtttttgacaccgcacatgtgtgttatggcttccttgatatgctctcgacagctctttggctggctttttcgcagagttcgttttgagaaggttatctttggcattttaacagtgatgtcaatacaaggttatgcaaacctccgtaatcaatggagcataataggagaatttaataatttgcctcaggaagaacttttacagtggatcaaatacagtaccacatcagatgctgtctttgcaggtgccatgcctacaatggcaagcatcaagctgtctacacttcatcccattgtgaatcatccacattacgaagatgcagacttgagggctcggacaaaaatagtttattctacatatagtcgaaaatctgccaaagaagtaagagataaattgttggagttacatgtgaattattatgttttagaagaggcatggtgtgttgtgagaactaagtttatacttcaagatggacaagaagttctatcagctgctgagaaatgatgccagatggtaactcagatatacaagaaatactcaaatgcgctggaaatggcctggttgcagtatgcttgaaatctgggatgtggaagacccttccaatgcagctaaccctcccttatgtagcgtcctgctcgaagacgccaggccttacttcaccacagtatttcagaatagtgtgtacagagtattgaaggttaactgagaaggatactacccattttactatggcacaatgccgtgtgtcaaaaacaatcaccctttggcttattcacattaataaaaatcacaagctttaataacagacacttaaaaataagataaaaatggattggaaatttttctgattactaaaaggtaaattacttttctgttcattgaatgtcagccttattaagcttgtcatataagttattaaatcattcatgtcatactgcataaacaaatgttcatttcagaattttaaagagaaatgtatataaaagaacaatgaattttaataaatcaggggtatgtaagtcctttttcatccaactaggtgaattgcttcagattttctctagtaccagagggtacctcctcaaactctttgaaccacttaaggcagaagaatgcaagctctgaaatgacatccttaaaatgctgatactggtcacagcctctttacctctgtgaggaaattgtaacagtgtgtcttttaaggtgtttttattttaccagcccttaagaaagatctttaataccttttaatacttttttttaataatttcaagttgaagtgtttttaaaaacactttgttttgtaatgttttgaatctcttgagatgtgtttaccccactagatacatatttgccactggttagttctccatctaagctcaagaggttattcatctctctttagattccagtggtttttcttttaacatccaggtaaaatagaaactgctatggtatacaaccaagttttggggttaaacataatcagaaaagaaaatccagttaaatttatgaagtgagattttcagatcctagatcttgaataaaggaaaggtcttttcatcttgatggccccaaagcttgttgatcatggtctttatttctggccactatcttcttaaataatatatttttaagccctcatttatttttggttttgggtgaggaaagtcatgttttctaagtcctctcccctaataaaacctacccaacaatagtgctttgaaaagtggtagttatcttgaagatactcttgccaaatgcaaagataaacattctttttgtctgctttataaatatgaaatatgccagatctgtagtattttaatgtgcatctactttaaatgagtcatcttggggtttttataattcccttatgttctcgcccctctacacttgaaataacaaaatgccttaattttatggattagttctcttatagtagacaggcagctatatgcagcaaaaccaataaagttatttttcaactttcatagttgtaaaatattttataacagaatacaaaacagctaagaaaacatgccacattttattttagcattttcaaataatttgtttttggtgtaagcacaggataaaaaaggagagcgtcaaagaaaagagacataacacctaacattcataaaaattaacaaagtatattttggatgatgtttttacaggaaatattttaaataagttggtagaacttttaaaatggtactgtattagctaataaaatattcagtacaaatatatgtttggatttatgcattaaaaaactaataaaattatttccaactttaaaaaaaaaaaaaaaaaaa

SEQ ID NO: 119 - protein, 171 aa, Homo sapiens
MRLKMFLTPHMCVMASLICSRQLFGWLFRRVRFEKVIFGILTVMSIQGYANLRNQWSIIGEFNNLPQEELLQWIKYSTTSDAVFAGAMPTMASIKLSTLHPIVNHPHYEDADLRARTKIVYSTYSRKSAKEVRDKLLELHVNYYVLEEAWCVVRTKFILQDGQEVLSAAEK

SEQ ID NO: 120 - DNA, 1502 bp, Homo sapiens
gtcgacgggaacgtagaactctccaacaataaatacatttgataagaaagatggctttaaaagtgctactagaacaagagaaaacgtttttcactcttttagtattactaggctatttgtcatgtaaagtgacttgtgaaacaggagactgtagacagcaagaattcagggatcggtctggaaactgtgttccctgcaaccagtgtgggccaggcatggagttgtctaaggaatgtggcttcggctatggggaggatgcacagtgtgtgacgtgccggctgcacaggttcaaggaggactggggcttccagaaatgcaagccctgtctggactgcgcagtggtgaaccgctttcagaaggcaaattgttcagccaccagtgatgccatctgcggggactgcttgccaggattttataggaagacgaaacttgtcggctttcaagacatggagtgtgtgccttgtggagaccctcctcctccttacgaaccgcactgtgccagcaaggtcaacctcgtgaagatcgcgtccacggcctccagcccacgggacacggcgctggctgccgttatctgcagcgctctggccaccgtcctgctggccctgctcatcctctgtgtcatctattgtaagagacagtttatggagaagaaacccagctggtctctgcggtcacaggacattcagtacaacggctctgagctgtcgtgtcttgacagacctcagctccacgaatatgcccacagagcctgctgccagtgccgccgtgactcagtgcagacctgcgggccggtgcgcttgctcccatccatgtgctgtgaggaggcctgcagccccaacccggcgactcttggttgtggggtgcattctgcagccagtcttcaggcaagaaacgcaggcccagccggggagatggtgccgactttcttcggatccctcacgcagtccatctgtggcgagttttcagatgcctggcctctgatgcagaatcccatgggtggtgacaacatctctttttgtgactcttatcctgaactcgctggagaagacattcattctctcaatccagaacttgaaagctcaacgtctttggattcaaatagcagtcaagatttggttggtggggctgttccagtccagtctcattctgaaaactttacagcagctactgatttatctagatataacaacacactggtagaatcagcatcaactcaggatgcactaactatgagaagccagctagatcaggagagtggcgctatcatccacccagccactcagacgtccctccaggtaaggcagcgactgggttccctgtgaacacagcactgacttacagtagatcagaactctgttcccagcataagatttgggggaacctgatgagttttttttttgcatctttaataatttcttgtatgttgtagagtatgttttaaaataaatttcaagtattttttttaaaaactaaaaaaaaaaaaaaaaaaaaaaaaaaaaaaa

SEQ ID NO: 121 - protein, 423 aa, Homo sapiens [sequence omitted]

SEQ ID NO: 122 - DNA, 2496 bp, Homo sapiens
aggtctagaattcaatcgggagagagatactgcctggttcttacagacacagattatgtcatccttgcagccttcacccaaagttgctccctccttctagggcattttgttttcctacttaataccaagtgtcagcatgttagtaataaacaggtgtctctaccattagtcaaaggtgggagttaagcctttcatctttgtagctttctccagtacctaaccatgatttacttcatgggaagtccctcaaagtactattaattatcctgtgttctcctgccttgcctcttaacaaaaattctgctgttcctgattatttccattttaccagtgttttgttccttttctatccaggcagcataattcgttgtatgaggcgcctggaagaattgcttcgacaaatgtgtcaagcagcaaaagccattggaaacactgagctggaaaataaatttgcagaaggtcagtatcaaatggataagctgtttctaatttagacaaatttggtgaagcaaatcttgagccctggaccacaacctaggaggacgttttgagatgttcttcactgcattgtcatggagagctatctactagtgtttctataaaatttagtgtgttgggggaaaagttgagattttatatatacatgcatttacgtatattaattgtacgctaactacatgccaggcattgttttaagcactaggggatatagtgaacaaaaagacacccctgccttcatggagtttacattctagtgtggggagatagacaataagtcaacaattaaatttatcagatggtgataagtctgtaagataaaaacaaagcagaaaagacaatagaattggcagatggatatgatggtctagggcctccacagggaaggtggcagttaaggcctttggtgtatgcttgatgtacgtgaacaccagcaagaggccagtgtggctggagcagaatgggcagtgagtagaggagtaggggttgaagtgagaaaggaaatgattccatagttcccagccccaggcttctcaactctgcagtgactggaggtgtggagtttgggagtgacctgccccaacttggactttacaaggtaattgttgctcttacattcagggcaagtctgtagagtagcacagtagaagtggggagaacagatcaagaaaggatggctaaaccaaggtggtagtaatggagtggtggtgggggggcaaggggcataagcttcagtcctgtctactgactcttgaccaagaaaaaggactaagttaatcaaagaatataaccacattgttgctgagtcagtcaatgctagtgatttctgcaaacaacttagtgtcctaagaagaggtttgcaaaaactggctgatatttccacagttgataaatgtaggcctgtttaatgactcagaatttaagtgtaggtgtcaaagttttaaaaataatttgtaatcaaaatttgtatttgtccttccttggcctagaaagaatttcacatgggaactgaaaaaaaagtgtcttctgatataggttgaaaatccaagtctttggattttacatttctttagacagtttagtccttccctataattttttttatttttatttttatttatttatttagtgtgtggagacagagtttcgctcttgttgccccaggctggagtgcaatggcgcaatctcggctcactgcaacctccacatccacctcccaggttcaagcgattctcctgcctcagcctcctgagtagctgaactacaggcatgcgccaccacgcccggctgattttgtatttctagtagagacggggtttctccatgttggtcgggctgctcttgaactcctgacctcaggtgatccgctcgcctcggcctcccagagtgctgggattacaggcatgagctactgcgcccgacctagcatttatctttttaaacagttctagacacctctttcctggccagcccccatggagtatttcagagtcaaaagaacaggggtctggcttgtatgttttccacctcacagaggtggctgcaaattcctctaggtgttcagcaaggtgtttgactttctaggctgctcgcttaccagttgaatcagggttggtatactggctttaaaatttcggtagaggcaagttaggtgttttgtggtcttgaaagtttaaaccttactttcttttctcttaggaatcaccaaaatcaagagagatattgtgtttgctgccagcctctacttgtagagtcagctaaaggaatgtgagattttaaattattgaccacctgtttgattacagttgactacaaatgcctgcaagtgtggatttggttctcccatacattttaatatgtattatatttaaatcaaacatcattcatagaaagcatataacatacatgtttatacataagcataacatttttttaataaaaatgtatacaggtggggcaaaaaaaaaaaaaaaaaaaaaaaaaaaaaaaaaaaaaaaa

SEQ ID NO: 123 - protein, 133 aa, Homo sapiens
MWLYSLINLVLFLGQESVDRTEAYAPCPPTTTPLLPPWFSHPFLICSPHFYCATLQTCPECKSNNYLVKSKLGQVTPKLHTSSHCRVEKPGAGNYGIISFLTSTPTPLLTAHSAPATLASCWCSRTSSIHQRP

SEQ ID NO: 124 - DNA, 789 bp, Homo sapiens
ctctcctctggctactgggtgctcgtggtgcattttactcggagagaggccatcaagcagatcgaggtgctgcagcacgtggccaccaacctggggcgcagccgtgcctggctgtacctggccctcaacgaraactcccttggararactacctgcggttgttccargaraaacctgggcctgctgcataagtactacgtcaagaatgccctggtctgcagccacgatcacctgacgctcttcctgaccttggtgtccgggctagagttcattcgtttcgagctggatctggatgccccttacctagacctggccccctacatgcccgactactacaaacctcagtacctgctggactttgaagaccgccttcccagctcggtccacggctcagacagtctgtccctcaactctttcaactccgtcacctccaccaacctggagtgggatgacagtgcgattgccccatctagtgaggattatgattttggagatgtgtttccagcagtgccgtctgtacccagcacagactgggaagatggagacctcacagacacggtcagtggtccccgctccacagcctccgacctgaccagcagcaaggcctccaccaggagccccacccagcgccagaaccccttcaacgaggagccggcagagactgtgtcctcctctgacaccacccccgtgcacaccacctctcaggagaaggaggaggcccaggccctggacccgccggatgcctgcacggagctcgaggtcatcagggtcaccaaaaaaaaaaaaaaa

SEQ ID NO: 125 - protein, 151 aa, Homo sapiens
MPDYYKPQYLLDFEDRLPSSVHGSDSLSLNSFNSVTSTNLEWDDSAIAPSSEDYDFGDVFPAVPSVPSTDWEDGDLTDTVSGPRSTASDLTSSKASTRSPTQRQNPFNEEPAETVSSSDTTPVHTTSQEKEEAQALDPPDACTELEVIRVT

SEQ ID NO: 126 - DNA, 3444 bp, Homo sapiens
agagacccaaagccaaaactcagctgacaggaatgtttcaaaggacacaaagagagatgtggactcaaagtcaccggggatgcctttatttgaagcagaggaaggagttctatcacgaacccagatatttcctaccactattaaagtcattgatccagaatttctggaggagccacctgcacttgcatttttatataaggatctgtatgaagaagcagttggagagaaaaagaaggaagaggagacagcttctgaaggtgacagtgtgaattctgaggcatcatttcccagcagaaattctgacactgatgatggaacaggaatatattttgagaagtacatactcaaagatgacattctccatgacacatctctaactcaaaaggaccagggccaaggtctggaagaaaaacgagttggtaaggatgattcataccaaccgatagctgcagaaggggaaatttggggaaagtttggaactatttgcagggagaagagtctggaagaacagaaaggtgtttatggggaaggagaatcagtagaccatgtggagaccgttggtaacgtagcgatgcagaagaaagctcccatcacagaggacgtcagagtggctacccagaaaataagttatgcggttccatttgaagacacccatcatgttctggagcgtgcagatgaagcaggcagtcagggtaatgaagtcggaaatgcaagtccagaggtcaatctgaatgtcccagtacaagtgtccttcccggaggaagaatttgcatctggtgcaactcatgttcaagaaacatcactagaagaacctaaaatcctggtcccacctgagccaagtgaagagaggctccgtaatagccctgttcaggatgagtatgaatttacagaatccctgcataatgaagtggttcctcaagacatattatcagaagaactgtcttcagaatccacacctgaagatgtcttatctcaaggaaaggaatcctttgagcacatcagtgaaaatgaatttgcgagtgaggcagaacaaagtacacctgctgaacaaaaagagttgggcagcgagaggaaagaagaagaccaattatcatctgaggtagtaactgaaaaggcacaaaaagagctgaaaaagtcccagattgacacatactgttacacctgcaaatgtccaatttctgccactgacaaggtgtttggcacccacaaagaccatgaagtttcaacgcttgacacagctataagtgctgtaaaggttcaattagcagaatttctagaaaatttacaagaaaagtccttgaggattgaagcctttgttagtgagatagaatccttttttaataccattgaggaaaactgtagtaaaaatgagaaaaggctagaagaacagaatgaggaaatgatgaagaaggttttagcacagtatgatgagaaagcccagagctttgaggaagtgaagaagaagaagatggagttcctgcatgagcagatggtccactttctgcagagcatggacactgccaaagacaccctggagaccatcgtgagagaagcagaggagcttgatgaggccgtcttcctgacttcgtttgaggaaatcaatgaaaggttgctttctgcaatggagagcactgcttctttagagaaaatgcctgctgcgttttccctttttgaacattatgatgacagctcggcaagaagtgaccagatgttaaaacaagtggctgttccacagcttcctagattagaacctcaggaaccaaattctgccaccagcacaacaattgcagtttactggagcatgaacaaggaagatgtcattgattcatttcaggtttactgcatggaggagccacaagatgatcaagaagtaaatgagttggtagaagaatacagactgacagtgaaagaaagctactgcatttttgaagatctggaacctgaccgatgctatcaagtgtgggtgatggctgtgaacttcactggatgtagcctgcccagtgaaagggccatttttaggacagcaccctccacccctgtgatccgcgctgaggactgtactgtgtgttggaacacagccactatccgatggcggcccaccaccccagaggccacggagacctacactctggagtactgcagacagcactctcctgagggagagggcctcagatctttctctggaatcaaaggactccagctgaaagttaacctccaacccaatgataactactttttttatgtgagggccatcaatgcatttgggacaagtgaacagagtgaagctgctttcatctccaccagaggaaccagatttctcttgttgagagaaacagctcatcctgctctacacatttcctcaagtgggacagtgatcagctttggtgagaggagacggctgacggaaatcccgtcagtgctgggtgaggagctgccttcctgtggccagcattactgggaaaccacagtcacagactgcccagcatatcgactcggcatctgctccagctcggctgtgcaggcaggtgccctaggacaaggggagacctcatggtacatgcactgctctgagccacagagatacacatttttctacagtggtattgtgagtgatgttcatgtgactgagcgtccagccagagtgggcatcctgctggactacaacaaccagagattatcttcatcaacgcagagagcgagcagttgctcttcatcatcaggcacaggtttaatgagggtgtccaccctgcctttgccctggagaaacctggaaaatgtactttgcacctggggatagagcccccggattctgtaaggcacaagtgatccttggctttcagaatttgcaagaacagcgatttgaattttgggggggtctgctgttcattcctttaggtgctatacattattcaaaaagtctcccgcgcatttgcactaatgatggctgcatgcatagcaatcagcatgtgagcaaaatcgacaagaaaaccttgactttacagagcagtgtgtgagtaaacagaatgaaaacaacaacctccactctttagtttatataagtttgagttctttcctaaattaaaagatctacacttgagttgggaaccgaaagagaaaaatggacttccatctgttttactggtaaaggaaatcctctgatggacaggtcagagtgaaggaaggttgtgctggtaagacatctctgacgaagagccatggatgctttccacaaaatgtcacctcgctgcactaaaggatgatgaatcctaatcattaaaggaattgtttcagctgatttaaatttataatgaactcttttgtaataatgtatactgtagaacatgagtctctcctccctaaaattttaaatgtagaaaagtgctatatattagaaatttccattttgttaaataaatggttagagtctataaaaaaaaaaaaaaa

SEQ ID NO: 127 - protein, 905 aa, Homo sapiens
MPLFEAEEGVLSRTQIFPTTIKVIDPEFLEEPPALAFLYKDLYEEAVGEKKKEEETASEGDSVNSEASFPSRNSDTDDGTGIYFEKYILKDDILHDTSLTQKDQGQGLEEKRVGKDDSYQPIAAEGEIWGKFGTICREKSLEEQKGVYGEGESVDHVETVGNVAMQKKAPITEDVRVATQKISYAVPFEDTHHVLERADEAGSQGNEVGNASPEVNLNVPVQVSFPEEEFASGATHVQETSLEEPKILVPPEPSEERLRNSPVQDEYEFTESLHNEVVPQDILSEELSSESTPEDVLSQGKESFEHISENEFASEAEQSTPAEQKELGSERKEEDQLSSEVVTEKAQKELKKSQIDTYCYTCKCPISATDKVFGTHKDHEVSTLDTAISAVKVQLAEFLENLQEKSLRIEAFVSEIESFFNTIEENCSKNEKRLEEQNEEMMKKVLAQYDEKAQSFEEVKKKKMEFLHEQMVHFLQSMDTAKDTLETIVREAEELDEAVFLTSFEEINERLLSAMESTASLEKMPAAFSLFEHYDDSSARSDQMLKQVAVPQLPRLEPQEPNSATSTTIAVYWSMNKEDVIDSFQVYCMEEPQDDQEVNELVEEYRLTVKESYCIFEDLEPDRCYQVWVMAVNFTGCSLPSERAIFRTAPSTPVIRAEDCTVCWNTATIRWRPTTPEATETYTLEYCRQHSPEGEGLRSFSGIKGLQLKVNLQPNDNYFFYVRAINAFGTSEQSEAAFISTRGTRFLLLRETAHPALHISSSGTVISFGERRRLTEIPSVLGEELPSCGQHYWETTVTDCPAYRLGICSSSAVQAGALGQGETSWYMHCSEPQRYTFFYSGIVSDVHVTERPARVGILLDYNNQRLSSSTQRASSCSSSSGTGLMRVSTLPLPWRNLENVLCTWG

SEQ ID NO: 128 - DNA, 1199 bp, Homo sapiens
rgacttgttgccagtgataccaaaacagacttttcccaagcagtgcctcacatgtctgctggtgtggctttgggattctcctgccccacccccccgtccatggcagccccctccccaaggctttgctcacacctgagacaggaaggaggaaggggatccaataggaatatgggccccggaggggaagtcatgcacccccaagccaccaccccccagccttccacgcacatctcctggytggaagagagccctccaaaaaggggacacaggctgccccggcccctcaactgcatccacaccccatcctctcatcttgggtcccagccaggcccccccaaaaccaaagccccctcaagtcctggggtcccagcctgtgcccccagcttcctgcccacccagccctgagcattctcacacagagaaagaacaagcaagggctccagggggacaggatggggcagggcatacagtggggggtgggggggcagctgggaggagggagggacaaaacaaaacattttcctttgggttttttttttctttcttttttctcccctttactctttgggtggtgttgcttttcctttccttttccctttgagatttttttgttgttgtttcctttttgtattttactgatatcaccaggatagtttactctccttctagctttctgcttaccgcacactggataacacacacatacacacccacaaaaatgctcatgaacccaatccggagaaggttccagcaggtcccccaccctcccctcctcctcctacttctcctcttgacagcgaggacaggagggggacaaggggacacctgggcagacccgccggctctccccccaccccaccccgcccctcacatcatactccaatcataaccttgtatattacgcagtcattttggttttcgcggacgcgcctacctaagtaccatttacagaaagtgactctggctgtcattattttgtttatttgttccctatgcaaaaaaaaaatgaaaatgaaaaaagggggattccataaaagattcaataaaagacaaacaaaaaaaaaagaaaaaagaaaaaaatgtataaaaattaaacaagctatgcttcgactcaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 129 - protein, 56 aa, Homo sapiens; identical to SEQ ID NO: 44

SEQ ID NO: 130 - DNA, 839 bp, Homo sapiens
  unsure (245)
gaatgaaaatccaggtgtttgtcattcatcagcaacaggtgatccccattgcaggcagccggaaccgacgtctcctggaccactgagctggctgttctcattactgccctttccgcccaggctggcggtgactcaccgtgagacaagtcagctaggtgttcaggacagggatttcagagtatttttgtccaaagaggaaagggatgatttctacggatcactaccagttggtttactgttagctncatcgtgttgatcacaccaagtccttgccaatttggttttctaagtattttcacgccttctcctcgtgtccgcgtcactgctctgattcaggcccttgtcatttctcatctttgccattttagtagtttttggattgggctcccggctgctaattttgtccccttttccactatcttccacattgtcaccgcagtcatgtttctaaggcagaatctcactgtgcccctcatcgtgttgggtgacttstggtggcatcccgtcaccctcaggacaacctttcctggggcctgcccgctctgctcttgctgctgcctcgctgtccccctcctccctcctgtggtttatattccaggaattctgaattagttgcaccgtgctctcatatttactgcaagaatagaccagtggttctccagcttttctgcactctggaatcacctgggggtctttaaaaaacactgcctggctcctagtcctaaatttggagatttaactggacttacagtttttcaaagcaccccaaaagattctaatgtgcagcaaagtttgggaaccactggtatagactgtcttctgcttgttttcttgaaaaa

SEQ ID NO: 131 - protein, 56 aa, Homo sapiens
  UNSURE (17)
MFLRQNLTVPLIVLGDXWWHPVTLRTTFPGACPLCSCCCLAVPLLPPVVYIPGILN

SEQ ID NO: 132 - DNA, 2166 bp, Homo sapiens
ggcaagctactggcacctgctgctctcaactaacctccacacaatggtgttcgcattttggaaggtctttctgatcctaagctgccttgcaggtcaggttagtgtggtgcaagtgaccatcccagacggtttcgtgaacgtgactgttggatctaatgtcactctcatctgcatctacaccaccactgtggcctcccgagaacagctttccatccagtggtctttcttccataagaaggagatggagccaatttctatttacttttctcaaggtggacaagctgtagccatcgggcaatttaaagatcgaattacagggtccaacgatccaggtaatgcatctatcactatctcgcatatgcagccagcagacagtggaatttacatctgcgatgttaacaaccccccagactttctcggccaaaaccaaggcatcctcaacgtcagtgtgttagtgaaaccttctaagcccctttgtagcgttcaaggaagaccagaaactggccacactatttccctttcctgtctctctgcgcttggaacaccttcccctgtgtactactggcataaacttgagggaagagacatcgtgccagtgaaagaaaacttcaacccaaccaccgggattttggtcattggaaatctgacaaattttgaacaaggttattaccagtgtactgccatcaacagacttggcaatagttcctgcgaaatcgatctcacttcttcacatccagaagttggaatcattgttggggccttgattggtagcctggtaggtgccgccatcatcatctctgttgtgtgcttcgcaaggaataaggcaaaagcaaaggcaaaagaaagaaattctaagaccatcgcggaacttgagccaatgacaaagataaacccaaggggagaaagcgaagcaatgccaagagaagacgctacccaactagaagtaactctaccatcttccattcatgagactggccctgataccatccaagaaccagactatgagccaaagcctactcaggagcctgccccagagcctgccccaggatcagagcctatggcagtgcctgaccttgacatcgagctggagctggagccagaaacgcagtcggaattggagccagagccagagccagagccagagtcagagcctggggttgtagttgagcccttaagtgaagatgaaaagggagtggttaaggcataggctggtggcctaagtacagcattaatcattaaggaacccattactgccatttggaattcaaataacctaaccaacctccacctcctccttccattttgaccaaccttcttctaacaaggtgctcattcctactatgaatccagaataaacacgccaagataacagctaaatcagcaagggttcctgtattaccaatatagaatactaacaattttactaacacgtaagcataacaaatgacagggcaagtgatttctaacttagttgagttttgcaacagtacctgtgttgttatttcagaaaatattatttctctctttttaactactctttttttttattttagacagagtcgcttgagcccaggaggtggaggttgcagtgggccgagattgtgccactgcactccaacctgggtgacagagtgagattccatctgaaaaacaaaaacaaaaacagaaaacaaacaaacaaaaaacaaaaaatccccacaactttgtcaaataatgtacaggcaaacactttcaaatataatttccttcagtgaatacaaaatgttgatatcataggtgatgtacaatttagttttgaatgagttattatgttatcactgtgtctgatgttatctactttgaaaggcagtccagaaaagtgttctaagtgaactcttaagatctattttagataatttcaactaattaaataacctgttttactgcctgtacattccacattaataaagcgataccaatcttatatgaatgctaatattactaaaatgcactgatatcacttcttcttcccctgttgaaaagctttctcatgatcatatttcacccacatctcaccttgaagaaacttacaggtagacttaccttttcacttgtggaattaatcatatttaaatcttactttaaggctcaataaataatactcataaaaaaaaaaaaaaaaaaaaa

SEQ ID NO: 133 - protein, 387 aa, Homo sapiens
MVFAFWKVFLILSCLAGQVSVVQVTIPDGFVNVTVGSNVTLICIYTTTVASREQLSIQWSFFHKKEMEPISIYFSQGGQAVAIGQFKDRITGSNDPGNASITISHMQPADSGIYICDVNNPPDFLGQNQGILNVSVLVKPSKPLCSVQGRPETGHTISLSCLSALGTPSPVYYWHKLEGRDIVPVKENFNPTTGILVIGNLTNFEQGYYQCTAINRLGNSSCEIDLTSSHPEVGIIVGALIGSLVGAAIIISVVCFARNKAKAKAKERNSKTIAELEPMTKINPRGESEAMPREDATQLEVTLPSSIHETGPDTIQEPDYEPKPTQEPAPEPAPGSEPMAVPDLDIELELEPETQSELEPEPEPEPESEPGVVVEPLSEDEKGVVKA

SEQ ID NO: 134 - DNA, 2946 bp, Homo sapiens
tcgggctgccttatcgccaagctccttcaggagaacaaagaacaggccattaccctggagaagactggcaactgattttacccacaagcccaaacctcagggatttcagtatctactagtctgggtagatactttcacgggttgggcagaggccttcccctgtaggacagaaaaggcccaagaggtaataaaggcactagttcatgaaataattcccagattcggacttccccgaggcttacagagtgacaatagccctgctttccaggccacagtaacccagggagtatcccaggcgttaggtatacgatatcacttacactgcgcctgaaggccacagtcctcagggaaggtcgagaaaatgaatgaaacactcaaaggacatctaaaaaagcaaacccaggaaacccacctcacatggcctgctctgttgcctatagccttaaaaagaatctgcaactttccccaaaaagcaggacttagcccatacgaaatgctgtatggaagccccttcataaccaatgaccttgtgcttgacccaagacagccaacttagttgcagacatcacctccttagccaaatatcaacaagttcttaaaacattacaaggaacctatccctgagaagagggaaaagaactattccacccttgtgacatggtattagtcaagtcccttccctctaattccccatccctagatacatcctgggaaggaccctacccagtcattttatctaccccaactgcggttaaagtggctggagtggagtcttggatacatcacacttgagtcaaatcctggatactgccaaaggaacctgaaaatccaggagacaacgctagctattcctgtgaacctctagaggatttgcgcctgctcttcaaacaacaaccaggaggaaagtaactaaaatcataaatccccatggccctcccttatcatatttttctctttactgttcttttaccctctttcactctcactgcaccccctccatgccgctgtatgaccagtagctccccttaccaagagtttctatggagaatgcagcgtcccggaaatattgatgccccatcgtataggagtctttctaagggaacccccaccttcactgcccacacccatatgccccgcaactgctatcactctgccactctttgcatgcatgcaaatactcattattggacaggaaaaatgattaatcctagttgtcctggaggacttggagtcactgtctgttggacttacttcacccaaactggtatgtctgatgggggtggagttcaagatcaggcaagagaaaaacatgtaaaagaagtaatctcccaactcacccgggtacatggcacctctagcccctacaaaggactagatctctcaaaactacatgaaaccctccgtacccatactcgcctggtaagcctatttaataccaccctcactgggctccatgaggtctcggcccaaaaccctactaactgttggatatgcctccccctgaacttcaggccatatgtttcaatccctgtacctgaacaatggaacaacttcagcacagaaataaacaccacttccgttttagtaggacctcttgtttccaatctggaaataacccatacctcaaacctcacctgtgtaaaatttagcaatactacatacacaaccaactcccaatgcatcaggtgggtaactcctcccacacaaatagtctgcctaccctcaggaatattttttgtctgtggtacctcagcctatcgttgtttgaatggctcttcagaatctatgtgcttcctctcattcttagtgccccctatgaccatctacactgaacaagatttatacaattatgtcatatctaagccccgcaacaaaagagtacccattcttccttttgttataggagcaggagtgctaggtgcactaggtactggcattggcggtatcacaacctctactcagttctactacaaactatctcaagaactaaatggggacatggaacgggtcgccgactccctggtcaccttgcaagatcaacttaactccctagcagcagtagtccttcaaaatcgaagagctttagacttgctaaccgctgaaagagggggaacctgtttatttttaggggaagaatgctgttattatgttaatcaatccggaatcgtcactgagaaagttaaagaaattcgagatcgaatacaacgtagagcagaggagcttcgaaacactggaccctggggcctcctcagccaatggatgccctggattctccccttcttaggacctctagcagctataatattgctactcctctttggaccctgtatctttaacctccttgttaactttgtctcttccagaatcgaagctgtaaaactacaaatggagcccaagatgcagtccaagactaagatctaccgcagacccctggaccggcctgctagcccacgatctgatgttaatgacatcaaaggcacccctcctgaggaaatctcagctgcacaacctctactacgccccaattcagcaggaagcagttagagcggtcgtcggccaacctccccaacagcacttaggttttcctgttgagatgggggactgagagacaggactagctggatttcctaggctgactaagaatccctaagcctagctgggaaggtgaccacatccacctttaaacacggggcttgcaacttagctcacacctgaccaatcagagagctcactaaaatgctaattaggcaaaaacaggaggtaaagaaatagccaatcatctattgcctgagagcacagcaggagggacaatgatcgggatataaacccaagtcttcgagccggcaacggcaaccccctttgggtcccctccctttgtatgggagctctgttttcatgctatttcactctattaaatcttgcaactgcaaaaaaaaaaaaaaaaaa

SEQ ID NO: 135 - protein, 538 aa, Homo sapiens [sequence omitted]

SEQ ID NO: 136 - DNA, 1240 bp, Homo sapiens
gccatccccatcaagcaggggatcctgctaaagcggagcggcaagtccctgaacaaggagtggaagaagaagtatgtgacgctctgtgacaacgggctgctcacctatcaccccagcctgcatcttggtgcgctgtctgtgccctctgccaacagtggaggcagcgaggatgaagaggagtggcaaggggtgtcttggatgtggaagaaagtgtgggttgtggggttgggctgggttttggtttcagtagaggaaacacagccagctggagagcagagctcaggggggttggtggcttttcagagtcacccggctggtggctgagctaagacttggacccatgactttggctctgagcattacccagatttttctgcacttgccaagagcacctccctctggggctggctgagagagtcatgtaagagttaatagcagggtgagtgttgtcaagtaaggagggagttgggcttgcctgcctggggctagggtgggtgtctgagcccccaggagtgcccctccatgccgcacttgttgcactgtgcgagtcttagaattcaccctgcaaggccaggcctggaagtcctggcatccagatcctgtcacaggccccgaagcatactgggctacacacggtgcaaaagcacgagtggaggcagggccggttgtggctccgtgcgctcacagctctccgtggagctctggcagagcccgcttcactttatgtcacgccgccaccacccccgccacactttccctccctccggggctgccacctcacctccttcatctcccctggccgccaccttccagcctgagcatgctcttcagttgccagcaatgagcaggccacctccctacctgtgagcagccgcttctctctggggctcttcaaaccctaaaccctggcaggaagcatgtcgaggaaggagctccggcaactccagaggctccgacagaactctgggctgagcctggctctcctctccagcaagggtctcgcctgagccccaagggcatcgggactggtgactcacctatggatgggggccggggagacaggacacacagaagatgagttcgtgggccagccctgagccccgcgcccgattctcgccggcccaagagagcccgccgcagcctcccccattttgcagccagcggagccattcacacaatcaccttctgttaattctatctgcaacatcaattaaattgtttgtagaaactaaaaaaaaaa

SEQ ID NO: 137 - protein, 114 aa, Homo sapiens [sequence omitted]

SEQ ID NO: 138 - DNA, 3153 bp, Homo sapiens
taatcatgcctcttggaagtaagttaacgggcgtgattgtggaaaatgaraatattaccaaagaaggtggcttaktggacatggccaagaaagaaaatgacttaaatgcagagcccaatttaaagcagacaattaaagcaacagtagaraatggcaagaaggatggcattgctgttgatcatgttgtaggcctgaatacagaaaaatatgctgaaactgtcmaacttaagcataaaagaascccaggtaaagtaaaagacatwtcmattgatgttgaaagaaggaatgaaaacagtgaggtagacaccagtgctggaagtggctctgcaccctctgttttacaccaaaggaacggacaaactgaggatgtggcaactgggcctaggagagcagaaaagacttctgttgccactagtactgaagggaaggacaaagatgtcaccttaagtccagtgaaggctgggcctgccacaaccacttcttcagaaacaagacaaagtgaggtggctttgccttgcaccagcattgaggcagatgaaggcctcataataggaacacattccagaaataatcctcttcatgttggtgcagaagccagtgaatgcactgtttttgctgcagctgaaaaaggtggggctgttgtcacagagggatttgctgaaagtgaaaccttcctcacaagcactaaggaaggggaaagtggggagtgtgctgtggctgaatctgaggacagagcagcagacctactggctgtgcatgcagttaaaatcgaagccaatgtaaatagcgttgtgacagaggaaaaggatgatgctgtaaccagtgcaggctctgaagaaaaatgtgatggttctttaagtagagactcagaaatagttgaaggaactattacttttattagtgaagttgaaagtgatggagcagttacaagtgctggaacagagataagagcaggatctataagcagtgaagaggtggatggctcccagggaaatatgatgagaatgggtcccaaaaaagaaacagagggcactgtgacatgtacaggagcagaaggcagaagtgataactttgtgatctgctcagtaactggagcagggccccgggaggaacgcatggttacaggtgcaggtgttgtcctgggagataatgatgcaccaccaggaacaagtgccagccaagaaggagatggttctgtgaatgatggtacagaaggtgagagtgcagtcaccagcacggggataacagaagatggagaggggccagcaagttgcacaggttcagaagatwgcakcgaaggctttgctataagttctgaatcggaagaaaatggagagagtgcaatggacagcacagtggccaaagaaggcactaatgtaccattagttgctgctggtccttgtgatgatgaaggcattgtgactagcacaggcgcaaaagaggaagacgaggaaggggaggatgttgtgactagtactggaagaggaaatgaaattgggcatgcttcaacttgtacagggttaggagaagaaagtgaaggggtcttgatttgtgaaagtgcagaaggggacagtcagattggtactgtggtagagcatgtggaagctgaggctggagctgccatcatgaatgcaaatgaaaataatgttgacagcatgagtggcacagagaaaggaagtaaagacacagatatctgctccagtgcmaaagggattgtagaaagcagtgtgaccagtgcagtctcaggaaaggatgaagtgacaccagttccaggaggttgtgagggtcctatgactagtgctgcatctgatcaaagtgacagtcagctcgaaaaagttgaagataccactatttccactggcctggtcgggggtagttacgatgttcttgtatctggtgaagtcccagaatgtgaagttgctcacacatcaccaagtgaaaaagaagatgaggacatcatcacctctgtagaaaatgaagagtgtgatggtttcatggcaactacagccagtggtgatattaccaaccagaatagcttagcagggggtaaaaatcaaggcaaagttttgattatttccaccagtaccacaaatgattacacccctcaggtaagcgcaattacagatgtggaaggaggtctttcagatgctctgagaactgaagaaaatatggaaggtaccagagtaaccacagaagaatttgaggcccccatgcccagtgcagtctcaggagatgacagccaactcactgccagcagaagtgaagagaaagatgagtgtgccatgatttccacaagcataggggaagaattcgaattgcctatctccagtgcaacaaccatcaagtgtgctgaaagtttcagccggttgctgcagcagtggaagaaagggctacaggtccagtcttgataagcaccgccgactttgaggggcctatgcccagtgcgcccccagaagctgaaagtcctcttgcctcaaccagcaaggaggagaaggatgaatgtgctctcatttccactagcatagcagaagaatgtgaggcttctgtttccggtgtagttgttgaaagtgaaaatgagcgagctggcacagtcatggaagaaaaagacgggagtggcatcatctttacgagctcggtggaagactgtgagggcccagtgtccagtgctgtccctcaagaggaaggcgacccctcagtcacaccagcggaagagatgggtgacaccgccatgatttccacaagcacctctgaagggtgtgaagcagtcatgattggtgctgtcctccaggatgaagatcggctcaccatcacaagagtagaagacttgagcgatgctgccatcatctccaccagcacagcagaatgtatgccaatttccgccagcattgacagacatgaagagaatcagctgactgcagacaacccagaagggaacggtgacytgtcagccacagaagtgagcaagcacaagktccccatgcccagcytaattgctgagaataactgtcggtgtcctgggccagtcaggggaggcaaagaactgggtcccgtgttggcagtgagcaccgaggaggggcacaacgggccatcagtccacaagccctctgcagggcaaggccatcaagtgctgtttgtgcggaaaaaaaaaaaaaa

SEQ ID NO: 139 - protein, 800 aa, Homo sapiens
  UNSURE (24)
  UNSURE (73)
  UNSURE (79)
  UNSURE (429)..(430)
MPLGSKLTGVIVENENITKEGGLXDMAKKENDLNAEPNLKQTIKATVENGKKDGIAVDHVVGLNTEKYAETVXLKHKRXPGKVKDISIDVERRNENSEVDTSAGSGSAPSVLHQRNGQTEDVATGPRRAEKTSVATSTEGKDKDVTLSPVKAGPATTTSSETRQSEVALPCTSIEADEGLIIGTHSRNNPLHVGAEASECTVFAAAEKGGAVVTEGFAESETFLTSTKEGESGECAVAESEDRAADLLAVHAVKIEANVNSVVTEEKDDAVTSAGSEEKCDGSLSRDSEIVEGTITFISEVESDGAVTSAGTEIRAGSISSEEVDGSQGNMMRMGPKKETEGTVTCTGAEGRSDNFVICSVTGAGPREERMVTGAGVVLGDNDAPPGTSASQEGDGSVNDGTEGESAVTSTGITEDGEGPASCTGSEDXXEGFAISSESEENGESAMDSTVAKEGTNVPLVAAGPCDDEGIVTSTGAKEEDEEGEDVVTSTGRGNEIGHASTCTGLGEESEGVLICESAEGDSQIGTVVEHVEAEAGAAIMNANENNVDSMSGTEKGSKDTDICSSAKGIVESSVTSAVSGKDEVTPVPGGCEGPMTSAASDQSDSQLEKVEDTTISTGLVGGSYDVLVSGEVPECEVAHTSPSEKEDEDIITSVENEECDGFMATTASGDITNQNSLAGGKNQGKVLIISTSTTNDYTPQVSAITDVEGGLSDALRTEENMEGTRVTTEEFEAPMPSAVSGDDSQLTASRSEEKDECAMISTSIGEEFELPISSATTIKCAESFSRLLQQWKKGLQVQS

SEQ ID NO: 140 - DNA, 2426 bp, Homo sapiens
tctgttcccgagctggagctgcgttgggacccgtcggatcgtaaatcccatgtaaggtatctgccgtcggaagatttgaactttctaattggacacctaacacccacagtcctccaggtgggtcctaaggatcttaggagcaacgatggggggtcctaagccagggggggatgagggtctggctctcagtccccgcctcgcggggagtgcctcccccctctgcgatgggggtcctaagagccagtgggggaaccaggggctggctctcagtccctgcctcgcggggggtgcctccccccctgtgatgggggtactaacagccaggggcggaagaggggatagctctcagtccccaccttcgcggggggtgcctccccctcgtgcgatgggggtcctaagatccagggggggaagagggactggctctcagtccctgcctcgcggggggtgcctccccccctgcgatgggggtactcacagccaggggtggaagaggggatagctctcagtccccactctcgtggggggtgcctccccctcctgcgatgggggtcctcagagccgggggggaagagggtctggctctcagtaatcccacgtaaggtacctgctgtcggaagatttgaactttctacttggacaactaacacccacagtcctccaggtgggtcctaaggatcttaggatcaatgatggggggtcctaagccggtgggggaagagggtctggctctcagtccccgcctcgcggggggtgcctcccccctctgcgatgggggtcctaagagccagtgggggaaccaggggctggctctcaatccctgcctcgcgggggttactcccccctcctgcgatgggggtaccaacagccaggggcggaagaggggatagctctcagtccccaccctcgtggggttgcctccccctcctgcgatgggggtcctaagatcctggggaggaagagggactggctctcagtaatcccacctaaggtacctgccgtcggaagatttgaactttctacttggacaactaacacccacagtcctccaggtgggtcctaaggatcttaggatcaatgatggggggtcctaagccaggggggaagagggtctggcactcagtccctgcctcgcggggggtgcctccgcccccagcgatgggggtcctaagagcaaaggggggaagaggggctccctctcagtccccgcgtcgcgaggggtgcctccccccctgcgatggcggtgcaaagagccaggggaggaaagagggaggttcgcagtccccgcctcgcgggaattgcctccccccctgctatggtggtcccaagagccagggggggaagaggggttggctctgagtccccgcctcgcggggggtgcctccccccctgcgatgggagtcccaagagccagggggtaagaggggatggatctcagccatcacaaaatggggggcctttatgttcaggttttacccaagaatcagcttatttgcttcttgtactagcagggcagttgctgccaaggccctcaaataggggggccatcctttagcaaccctgtctagttgtttagagacgtaggctacgggcctcagccagggccccacagtttgggttaaaagtccagctgccatcttttctctctctgacgcatacaatggaaaaggctttgtcaggtcgggtgggctgccagaagattttcttgtaactcatgaaaaacttgctgttgttgggatccccatttcaaaagttccgggtccccgccccatttgtgacctcatacaaaggcttggctaatactgcagtttgggatccacagcctacaaaaccccacagctcctaagaattctctcacctgccttctgcccttaagctccggtagattgcaaataacctgctttctttctgttcccgagctgtgttcggacccgtcggatcgtaaatcccacgtaaggtcggaagatttgaactttctacttggacacctaacacccacagtcctccaggtacctgccgtcggaagatttgaacgttctacttggacaactaacacccacagtcctccagacagaaagacaacaggtacaaagccctaaggattataaaggtatgctgcttaccatcatcttagtgaccaaggcagcgaagctgtttctgtaccttggaacagtcttccctgacaagccagagaacagtgataaagccaccagccttgggatcaggactgaaaaggcaagagtgatggagatttctcctgcgctaagccaagagaaggtttcagcacttcagacagctcccaccgaagtagccgcgctcccagctgcttgcagatgttgaaaaggaaagcctcggtttgtcttgaggttgtcagcaggtgcaagacacgtaataaaatgcaatgtgttcctaaaaaaaaaaaaaaa

SEQ ID NO: 141 - protein, 75 aa, Homo sapiens [sequence omitted]

SEQ ID NO: 142 - DNA, 296 bp, Homo sapiens
ggtcccctctctctgcccctcccactccttttctacggcgatttgtctgtgtctggcccccacccactgcccatcccccattgttgtctggatgtggttctattttttatcggtctcctttcccctcctccccgttctcgcccccgccccaccccctgctcccactaccctttgtctcttgctctttcttgggcttctgtacaactcaacttgtatacactgtgtacacacaaccagccaaacgaaaacccaacggcaaacaaaaaaaaaaaaaaaaaaaaaaaaaaaaaaaaaaa

SEQ ID NO: 143 - protein, 57 aa, Homo sapiens
MWFYFLSVSFPLLPVLAPAPPPAPTTLCLLLFLGLLYNSTCIHCVHTTSQTKTQRQT

SEQ ID NO: 144 - DNA, 271 bp, Homo sapiens
gccccttccacctcttctcctatgacttkgaggactcctccctgtccaccaaggagaaggaagcagagtcccagaaggaaaacagatacagcaattttggcaataactcttatcactcctcaagaccctcatctggatccagtgtgcccaccacccccacatcatccgtctcacccccacaggaggccaggttggaaaggtcatcaccgagtggtcttctcacatcatccttcaggcagcaccaagagtcactggcaaaaaaaaaaaaaaa

SEQ ID NO: 145 - protein, 38 aa, Homo sapiens
VPTTPTSSVSPPQEARLERSSPSGLLTSSFRQHQESLA

SEQ ID NO: 146 - DNA, 1630 bp, Homo sapiens
  unsure (1622)
cctgacctcaggtgatctgcccgcctcggcctctgaaagtgctgggattataggcatgagccaacatgcctgacctgttatttattttaaattatatcaggaatacacacacacacacacacacacacacacacacacacacaacttataaagataatggtctccttggcactcccacccacccacccatccaaatttacacaagtaaatctgtaatcaatttggttagaagggatttattttaatatttttggggattgcttatgatgcagtataatttttagttatattagtagtaattggaaatgtgtatttttgtgactgaagtcaccttctaaataatttctagaataaaatttttatattgaagaagttggtcttaaccatttttttttcaggagcatgcattttgaaatcattctgtgggaagatgaaaacaaatttagttctatgtctcccctttttagagatgttgacactttccttaaatgtaccatgcatgatttgtctaccacccttttagcttgttatacttaaatcccagatctctgtcttcccatttcagtttctctagaatttctggctgcttccaatgggtcaaatttatgagtgaaccattaagaatcacttagtgtagaaataaaccatgggttaggagtttgaacactgcctaggttctgtttctgatttgattatgactcagctgtgtggccttgggaaaccaccttactggtatccctatccttgcagaagcaagagagttaatgatggttgacttaatctcttgtggttattatgaagatcagataagatacattaacacattttgccaactgaattaggttatttatttacatgtgtgtccatggacctggggatcaggtgctatgtctcagccttatctttgtttttaatcctgtgtctctaattgtgtttgtcagtaaaggagtgagtcatttaatggttgctagatgtttgagtaaaacaaacaagcaaacaaatggtaaattagtactatttctttttaaaaaatttttttttacattttaaaaattatagataaatacagagatgaggtctcaccatgttgcccagtctggtttcaaactcctaaactcaagtgatcctctctcctcagcctcccaaagtgctaggattacaggcgtgagccaccatgcctggccagtagtactatttctttgggaaaatatttagtagtagtcaacaaagttgagcatactgtgacctggcagttttgatgctaagtawatacccaacagaaatgcaaacatatacttaccaaaactcatgtccaagaatattcgtagaagcacaattcttatgatagcaaaaaggtagaaaacaacytaaatgtttytaagcagtagcataagagtaataccgtgtggtttgtttatacagtgagatcctgtacagccatgtaaaagaccaaaatattccctgtaacaatgagaatgaatctcctgtgcttgcttcggcagcacatacactaaaattggaacgatacagagattagcatggcccctgtgcaaggagaatgaatyttcgtaatgttcagcaaaagaagccagatataaatgaatattccattttataaaaanaaaaaaaa

SEQ ID NO: 147 - protein, 30 aa, Homo sapiens
MKTNLVLCLPFLEMLTLSLNVPCMICLPPF

SEQ ID NO: 148 - DNA, 456 bp, Homo sapiens
aagaaggagactgtaagcttgtttgtacaaaaacataccatacagagaaagctgaagacaaacaaaagttagaattcttgaaaaaaagcatgttattgaattatcaacatcactggattgtggataatatgcctgtaacgtggtgttacgatgttgaagatggtcaggttctgtaatcctggatttcctattggctgttacattacagataaaggccatgcaaaagatgcctgtgttattagttcagatttccatgaaagagatacattttacatcttcaaccatgttgacatcaaaatatactatcatgttgttgaaactgggtccatgggagcaagattagtggctgctaaacttgaaccgaaaagcttcaaacatacccatatagataaaccagactgctcagggccccccatggacataagtaacaaggcttctggggagataaaaattgca

SEQ ID NO: 149 - DNA, 519 bp, Homo sapiens
  unsure (4)
  unsure (12)
  unsure (28)
  unsure (35)..(36)
  unsure (51)
  unsure (63)..(65)
  unsure (90)
  unsure (111)..(112)
  unsure (123)
  unsure (136)
  unsure (148)
  unsure (157)
  unsure (161)
  unsure (204)
  unsure (239)
  unsure (305)
caantaataaancttttgtttccctcgncattgtnntcgttcccctgtccngccttgtttccnnngtcctgcaccaatatttccaaaccnaatacccaagcatacaatccnnactccaagctnggaattcgcccanagagaccgtcgngggaagaanttgnctggaaacttgttcatggtgatatataccgtcctccaagaaangggatgctgctatcagtctttctaggagccggganacagatattaattatgacctttgtgactctatttttcgcttgcctgggagttttgtcacctcccanccgaggagcgctgatgacgtgtgctgtggtcctgtgggtgctgctgggcacccctgcaggctatgtttctgccagattctataagtcctttggaggtgagaagtggaaaacaaatgttttattaacatcatttctttgtcctgggattgtatttgctgacttctttataatgaatctgatcctctggtcaacggcctctttggccctcgagaca

SEQ ID NO: 150 - protein, 89 aa, Homo sapiens
  UNSURE (18)
MTFVTLFFACLGVLSPPXRGALMTCAVVLWVLLGTPAGYVSARFYKSFGGEKWKTNVLLTSFLCPGIVFADFFIMNLILWSTASLALET

SEQ ID NO: 151 - DNA, 507 bp, Homo sapiens
ttcttcccatacacctttcccccataagatgtgtcttcaacactataaagcatttgtattgtgatttgattaagtatatatttggttgttctcaatgaagagcaaatttaaatattatgtgcatttgtaaatacagtagctataaaattttccatacttctaatggcagaatagaggaggccatattaaataatactgatgaaaggcaggacactgcattgtaaataggattttctaggctcggtaggcagaaagaattatttttctttgaaggaaataactttttatcatggtaattttgaaggatgattcctatgatgtgttcaccaggggaatgtggcttttaaagaaaatcttctattggttgtaactgttcatatcttcttacttttctgtgttgacttcattattcccatggtattggccttttaaactatgtgcctctgagtctttcaatttataaatttgtatcttaataaatattataaaaatgaaaaaaaaaaaaaa

SEQ ID NO: 152 - DNA, 1902 bp, Homo sapiens
gattagtctgaagccgccaccagccccaggcccccgtgcagaagaaaagcgggagggaacggcggaggccgccgctgccctgcaccgccctcctggaggccacttggagagtccggccccgaggaggccatggccacaagtgcccacagctggccccaggttgccagcgtcgctacagcccagaccaaggcagaataatctccggatgagctggtggcaccgctgagcctttggtctcaccagggcttcctgttgctggcaggcggggtggagcggagctgctgggaggctgctggataggagaggggtcacggctgcggaagaggaggttcttcgggacacccgtggatggacacggcaaggaaacaccaggccaaccacagctggggataaaatagcacaaccacaccctgccgtccagcgcctcccagcctgtgccccttcctagtaccaccagcaaccatcaatcccgtctcctcctgcctcctctcctgcaatccaccccgccacgactatcgccatggcagccctgatcgcagagaacttccgcttcctgtcacttttcttcaagagcaaggatgtgatgattttcaacggcctggtggcactgggcacggtgggcagccaggagctgttctctgtggtggccttccactgcccctgctcgccggcccggaactacctgtacgggctggcggccatcggcgtgcccgccctggtgctcttcatcattggcatcatcctcaacaaccacacctggaacctcgtggccgattgccagcaccggatgaccaagaactgctccgccgcccccaccttcctccttctaagctccatcctgggacgtgcggctgtggcccctgtcacctggtctgtcatctccctgctgcgtggtgaggcttatgtctgtgctctcagtgagttcgtggacccttcctcactcacggccagggaagagcacttcccatcagcccacgccactgaaatcctggccaggttcccctgcaaggagaaccctgacaacctgtcagacttccgggaggaggtcagccgcaggctcaggtatgagtcccagctctttggatggctgctcatcggcgtggtggccatcctggtgttcctgaccaagtgcctcaagcattactgctcaccactcagctaccgccaggaggcctactgggcgcagtaccgcgccaatgaggaccagctgttccagcgcacggccgaggtgcactctcgggtgctcgctgcaacaatgtgcgccgcttctttggctttgtggcgctcaacaaggatgatgaggaactgattgccaacttcccagtggaaggcaggcagccacggccacagtggaatgccatcaccggcgtctacttgtaccgtgagaaccagggcctcccactctacagccgcctgcacaagtgggcccagggtctggcaggcaacggcgcggcccctgacaacgtggagatggccctgctcccctcataaggagtgcttcccatgctatttggtaaatggcagtgattggtcccattctgaaccccactgcttgctcacatccatatcagaaggggatttttaaaaaactgttatcttcttggccaggggaaaggaccactaggcaatctggggtgtggacagacccagtagacaatggaagccccagccagctgggccaggtgacagtgaagctcaccagtgggctcatttatggtactatatgcagttaacatgtatctagctgcatagggacacccagcgcagcagtgcaccactgggaagtggcctccagtgcagcctctggccttattttatatatttaaatttttgataaagtttttcttactaaaaggaaaaaaaaaaaaaaaaaaaaaaaaaaaaaaaaaaaaaaaaaaaaaa

SEQ ID NO: 153 - protein, 262 aa, Homo sapiens
MAALIAENFRFLSLFFKSKDVMIFNGLVALGTVGSQELFSVVAFHCPCSPARNYLYGLAAIGVPALVLFIIGIILNNHTWNLVADCQHRMTKNCSAAPTFLLLSSILGRAAVAPVTWSVISLLRGEAYVCALSEFVDPSSLTAREEHFPSAHATEILARFPCKENPDNLSDFREEVSRRLRYESQLFGWLLIGVVAILVFLTKCLKHYCSPLSYRQEAYWAQYRANEDQLFQRTAEVHSRVLAATMCAASLALWRSTRMMRN

SEQ ID NO: 154 - DNA, 533 bp, Mus musculus
  unsure (32)
  unsure (38)
gggatatcccatacaggtatgaaaaaaccccntatgtnatagtgttctatagcacacaataccttatgaaggaagggtttsatgaatacatggcagaagacaatcatgaaagamttatyttgaggggytagaartaatgagtttggaggtgtgccccttaggtcctgartgtcctgggatccctmacccctaatttctctcccaragcatyatcccttctcagtattggtactacatgattgaactttccttctastggtccctgytcttcagcattgcctctgatgtcwagcgaaaggattttaaggaacagatcatccaccatgtggccactatcattctcctctgcttctcctggtttgccaattacgtccgggcagggaccctcatcatggctctgcatgacgcttctgactacctgctggagtctgccaagatgtttaactacgcgggatggaagaacacctgcaacaacctcttcattgtgttcgccatcgttttcatcatcactcggctggttatcatgcctttct

SEQ ID NO: 155 - protein, 44 aa, Mus musculus
MTLLTTCWSLPRCLTTRDGRTPATTSSLCSPSFSSSLGWLSCLS

SEQ ID NO: 156 - DNA, 313 bp, Mus musculus
  unsure (4)
  unsure (33)
  unsure (35)
  unsure (94)
  unsure (226)
aaanacaagtcaatgaagtgaaggagggtatgnanacatgcccctcaccataccccagggaccatggttcctaggatctcactgcctccctttntggccttcctgtcccctcccttcagctatgacagctggtgtggagtagaagggcaactagttctgttatttattgaacatttggggtttcagttgtaaagccacaactacaggtaggacctgatatttcggngagggaccatttcagaccaaaatgtactgttaattttttttaattaaagtatattaaaggttaaataaaaaaaaaaaaaaaaaaaaa

SEQ ID NO: 157 - DNA, 1212 bp, Mus musculus
attacaatcatttcaaattttgaatttttaagttgatgggctcttaagtggtccgttctgaatagaaaccaatttgctagtttcggttttgttttgttttgttttgttttgttttgttttgtttttttaaggaatcagatagccagaaaaaaaaatgctattgcttgttttcatgaacttcagttgtctctttttagtaaacccagtactttccacaaagtcttctctgaccttccccatcactggacggttcacccatcttcttctccaagtgtttatcccccagcccaagcctttcctgctgcaagccaagcctgctacatttgttacagaccaagcttatacacagctcgacaactgcactcccactgtaggctccggtgtgtactcttgtcttgtgttgggaaggggaagtgaagtgataagccagaatttttttcaggaggtttctgtcattgatcctgtgagtcacagtttactcttcagttcacaccatcatcaggacagatggctgttcacaggtatcagaaaagaaagtgaaaagttttggctgggggcagaatctagggacagccaccaagattagaatcataaaagatattgaaagtagcacccctctgctctctggccttggctttacctaaatcttgtgatttggagagtcacaatagcttatctgtaacccttgacactgggacttgccaaatgatctggttcaatactcccagaatgagcagagggtccaaagtggatccctgagccattttgaataattttatcaccactgacagagtacatcacctgagctgtctgtcatgtgagacccaagggacatttccaacttgctgcctgcccagcacatatgtctttcccagtttcttccttccccatcttcttcgtgctctctgctctccaccagggtggaggtatggatctgtgacagaggttcttgggaacagctcagcagatttttgagaccaatcaaatggcctcattaagaactttatctgtttggaaacatggcttccttcctggctctgctaaactgaaagctcatttgttgttgctgttgttgttgtttgtttgtccatttctctttaattctaatgttcacatcatgtcgtgctgtatgactctagaaagccttaatttacttccaccaagaaataaagcaatatgttggtaatctgaaaaaaaaaaaaaaaaaaaaaa

SEQ ID NO: 158 - protein, 143 aa, Mus musculus
MLLLVFMNFSCLFLVNPVLSTKSSLTFPITGRFTHLLLQVFIPQPKPFLLQAKPATFVTDQAYTQLDNCTPTVGSGVYSCLVLGRGSEVISQNFFQEVSVIDPVSHSLLFSSHHHQDRWLFTGIRKESEKFWLGAESRDSHQD

SEQ ID NO: 159 - DNA, 1818 bp, Mus musculus
gggtccacaggccgcagccatgggtagccgcgtgtcccgagaggagttcgaatgggtctacacggaccagccccacgccgcccggcgcaaggagatcttagcaaagtatccagagatcaagtccttgatgaaacctgaccacaatctgatctggattgtagccatgatgcttctcgtccagctggcttcattttacttagtcaaagatttggactggaaatgggtcatattttggtcctatgtctttggcagctgccttaaccactccatgactctggctatccatgagatttcccacaatttcccctttggccaccacaaggccctgtggaaccgctggtttggaatgtttgctaacctctctctcggagtgccatactcgatttcctttaagagataccacatggatcaccaccggtacctcggagcggataagattgatgtagatattcccaccgatttcgagggctggttcttctgcaccactttcaggaaatttgtctgggttatccttcagcctctcttttatgcttttagacccctgttcatcaatcccaaacccattacctatctggaaatcatcaatactgtgatccagatcacctttgacattatcatttattatgtgtttggagtgaaatctttagtctacatgttggcagccaccttgcttggcctgggtttgcacccaatttctgggcattttatagccgaacattacatgttcttgaagggacacgaaacctactcctattatgggcctctgaacttgctcaccttcaatgtgggctatcataacgagcaccatgacttccccaacgttcctgggaaaaacctgcccatggtgaggaagatcgcaagtgagtactacgatgacctcccgcactacaactcctggatcaaggtgctctatgactttgtgacggatgacacaataagtccctactcgcggatgaagaggcctccgaaagggaacgagattctggagtaaatatcacagccaaaggaattcctgtccacggcttcaaaataggtgatagagtggagtttttatctcgttaggcttatggtcagtgatgctcggaagctgggctggcatgattgccaactcagatctcggtgtcatcagaggctcctcagccttcagacagcccgacgacgacgcttgacgctagcggctgctgtgcttgctcactcccctgcactcacgagatgttattactgttactgagggcgtctttcctcgtgtctgtcatgctgtgagcaaatcataaaaagcttatattatttttcactataaagttactttattaaagcacctaataaactgaactgttaatcatatatattaataatattcattgcattattatatttctattgtattggtatacttaccagagtgctagggagaactttacaatttcatccagaaatttaattttgttaaaggcttgacttgagttcatatagtattgtgtgagttccctctggattcaatcatttgttcttagtggggaatggaaggcttataagtctcgggaaactgatgaaattctccatcacaaagtccaaagggtctttggacaagggtccaaacatgaagtgctttctggaggagcttttgtttagtccaacaggagtccaaggatgcagattagagttttgtgagtttgctgcccttgctgggctaggcatttcattgttgtaactgcttctgagtaactgatgatcctataagtaaccccaataaactctctggtttactaaaaaaaaaaaaaaaaaaaaaaaaaaaaaaaaaaaaaaaaaaaaaaaaaa

SEQ ID NO: 160 - protein, 323 aa, Mus musculus
MGSRVSREEFEWVYTDQPHAARRKEILAKYPEIKSLMKPDHNLIWIVAMMLLVQLASFYLVKDLDWKWVIFWSYVFGSCLNHSMTLAIHEISHNFPFGHHKALWNRWFGMFANLSLGVPYSISFKRYHMDHHRYLGADKIDVDIPTDFEGWFFCTTFRKFVWVILQPLFYAFRPLFINPKPITYLEIINTVIQITFDIIIYYVFGVKSLVYMLAATLLGLGLHPISGHFIAEHYMFLKGHETYSYYGPLNLLTFNVGYHNEHHDFPNVPGKNLPMVRKIASEYYDDLPHYNSWIKVLYDFVTDDTISPYSRMKRPPKGNEILE

SEQ ID NO: 161 - DNA, 750 bp, Mus musculus
gcctcccaagtgctgggattaaaggcgtgtgccaccatgccccacttcatatgttatatttttaatgaataaagagtggaaaaattatgtatcacatgtgttaatttggggagaagcgctttataacagagggcttactctcaattaaagagaacaaaggaaaatgtgttctacaggcagtgtatacctttgacctctgaaaaaacctatatagtttctcctacagacaccttgccagtaaccttacaggtcttataggagagcagatccaagttgccaggctgatctgcaagcacaaacatttgtcaagggaaagcacaggtcgttactttcagtacaaaatggttctttgctatggatggattctcttcttcttgccccatgtcctgttcccaaggaccgacttcctgcagcactgtggtggactcttctatgaggagacaacatctgggccttattcaatagcctgtggtgggtaatgtgttttgtcaagagctaaacagcaaatggatttaatttctgcttaacatggtcatagtcattctgaaatggctacagaaatattctctgtactagaaaaaggaatggaacgtggtgccaattgtctattttcctttatttattccctgtaagtctgtcagatgataaattgaacataacagtgattaaagagtcatgctaaaaaaaaaaaaaaaaaaaaaaaaaaaaaaaaaaaaaaaaaaaaaaaaaaaaaaaaaaaaaaaaaaaaaa

SEQ ID NO: 162 - protein, 45 aa, Mus musculus
MVLCYGWILFFLPHVLFPRTDFLQHCGGLFYEETTSGPYSIACGG

SEQ ID NO: 163 - DNA, 671 bp, Mus musculus
agatcagttagctccctgggtcggaacaaggtgaaaagcagctttcttgcttttgaaatcatctttgtgacaaggacacatggggtcagggtagggtgtccagttaaaatagtgtcactgcttagaaaggggtacttggattcctttagttagcttagctctgtctcttgtttcataaaacacactgggttagaatagaggctcctgcattacatggtttgtgtcactgttttttgttgggttttctttttggtttttcgagacagggtttctctgtatagccctggctgtcctagaactcactctgtagaccaggctggcctcgaactcagaaatctgcccgcttctgcctcccaagtgctgggattaaaggcatgtgtcacgttttaaattgatagttataacctcgatgccacgaatcctgcagtttctcctgtgctcctttctttgtgtcagatgggttaagggttatcagtttggggaagaattgtccttgtaccccctggaattatttttctcaaaaatccaagactccaaagaacatgggaaaaattgttctgtccacttttgacgttgaagattttggttatccttttcgtactttctatgtattttctatgtaaaattttacacaattaaaaatgtttttttgtctagtaaaaaaaaaaaaa

SEQ ID NO: 164 - protein, 58 aa, Mus musculus
MVCVTVFCWVFFLVFRDRVSLYSPGCPRTHSVDQAGLELRNLPASASQVLGLKACVTF

SEQ ID NO: 165 - DNA, 1351 bp, Mus musculus
  unsure (1334)
cagcgcgcggagccggcgtcccgttggcgcgctctggcctggcttcgggtcgtcgcttcggccccgaggagccgctcgctgtctccggagcggcggagaggatggtgcggggcagcccggggcccgccgcgcgccgccgcgagtgaacagggccaggccgcgggcgtccgcgggctcgarccgccagtctgcggggcggttgccgctggtgggaagcatgttcagtatcaaccccctggagaacctgaagctgtacatcagcagccggccgcccttggtggtttttatgatcagtgtcagcgccatggccatcgccttcctcaccctgggctacttcttcaagatcaaggagattaagtccccagaaatggctgaggattggaatacttttctgctccggtttaatgatttggacttgtgtgtatcagaaaacgagacactgaagcatctctccaacgataccaccacaccagagagcaccatgaccgtcgggcaggccagatcgtctacccagccgccccagtccctggaggagtcaggccccatcaatatttcagtggccattaccttgaccttggaccctctcaagccctttggagggtactctcgaaatgttacacacctgtactccaccatcctcgggcatcagattggattgtcaggcagggaagcccacgaggagatcaacatcaccttcaccctgcctgctgcctggaacgccgatgactgtgccctccatggccactgtgagcaggcggtgttcacagcatgcatgaccctcacagctgcccccggagtcttccccgtcactgttcagccacctcactgtatccccgacacatacagcaacgccacgctctggtacaagatcttcacaactgccagagatgccaacacgaaatatgctcaagactacaatcctttctggtgttataagggtgccattgggaaagtctaccatgctttaaatcccaaactcactgttgttgttccagatgacgaccgctcattaataaacctgcatctcatgcacaccagttacttccttttcgtgatggtgataacgatgttctgctatgcagtcatcaaaggcagacccagcaaactgcggcagagcaatcctgaattttgccmtgagaaggtggytctggctgacgcctaatcctacagctccccattttytgagagaccaagaaccatgatcattgcctgctgaatcggccagggcctggccactctgtgaatacatgatcttgcaatgttgggttattccagccaaagacatttcaagtgcctgtaactgatttgtccatatttataaacactgatctggnaaaaaaaaaaaaaaaaa

SEQ ID NO: 166 - protein, 314 aa, Mus musculus
  UNSURE (306)
  UNSURE (310)
MFSINPLENLKLYISSRPPLVVFMISVSAMAIAFLTLGYFFKIKEIKSPEMAEDWNTFLLRFNDLDLCVSENETLKHLSNDTTTPESTMTVGQARSSTQPPQSLEESGPINISVAITLTLDPLKPFGGYSRNVTHLYSTILGHQIGLSGREAHEEINITFTLPAAWNADDCALHGHCEQAVFTACMTLTAAPGVFPVTVQPPHCIPDTYSNATLWYKIFTTARDANTKYAQDYNPFWCYKGAIGKVYHALNPKLTVVVPDDDRSLINLHLMHTSYFLFVMVITMFCYAVIKGRPSKLRQSNPEFCXEKVXLADA

SEQ ID NO: 167 - DNA, 1130 bp, Mus musculus
  unsure (1108)..(1109)
  unsure (1111)
ctttcatatccacgatgcgttttctggccgccacgatcctgctgctggcgctggtcgctgccagccaggcggagcccctgcacttcaaggactgcggctctaaggtgggagttataaaggaggtgaatgtgagcccatgtcccaccgatccctgtcagctgcacaaaggccagtcctacagtgtcaacatcacctttaccagcggcactcagtcccaraacagcacggccttggtccacggcatcctggaagggatccgggtccccttccctattcctgarcctgacggttgtaagartggaatcaactgccccatccagaaagacaaggtctacagctacctgaataagcttccggtgaaraatgaatacccctctataaaactggtggtggaatggaaacttgaagatgacaaaaagaataatctcttctgctgggagatcccagttcagatcacaagctaggctccttggcgcctgtgtctgtgtgggctgagaggccatggacggagtgggggggaagaaacagaaatcagacccgaaatggaatcagtgccatatgatgaacagaatttcaagaatgctgttttatgccttttaacctccaaagcagtaactgcaagcctactactcttgagagcgcgctcagagccattgtcccccgagatagcctctggggaggcttcgggagggaaaggggaaactggaaagattagattggtgtccatggctgtttgctgttggattacaggcaggttccaccagacaggatgggacggggttgcttagggatgtcagataacttgacccagggctatggatccactgtgaaggatggcttcccagagtcttctggctgcctgggggtgttacctcccctgtttctaagtgcctcctgagtccccagcccctggcttatcagtcagatgagtctccttggtagcctctgccccatcgcttcagcagtagcgactagctytcctcggtatccagactggctgaggggcagtctgccgcagaaatttgtctctgagtggctgtgtctttgtggttagctctcgttctttggtagttttcattaaagccaatacttggttgcaaaaaaaaanngnaaaaaaaaaaaaaaaaaaa

SEQ ID NO: 168 - protein, 149 aa, Mus musculus
  UNSURE (95)
MRFLAATILLLALVAASQAEPLHFKDCGSKVGVIKEVNVSPCPTDPCQLHKGQSYSVNITFTSGTQSQNSTALVHGILEGIRVPFPIPEPDGCKXGINCPIQKDKVYSYLNKLPVKNEYPSIKLVVEWKLEDDKKNNLFCWEIPVQITS

SEQ ID NO: 169 - DNA, 3070 bp, Homo sapiens
cacagtaggtgtatatatttatggactacatgagatacaggcatgcaatgcataataatcacatcatggaaaattgggtattcatcacctcaagcatttatcctttgtgttacagataatccacttctactcttttacttattttaaagtgtacaattaaactatgattgactatagtctccttgttgtgctgtcacatactaggtcttattcattccttctatttttttgtacccattaactatccccacctccccactgcagttcccagcctctggtaaccatccttctactctgtttccatgagctcaattgttttgatttttagatcccacaaataagtgagaacacgtgatgtttgtgtttctgtgcctggcttatttcacttaatataatgacctccagtttgatccacgttgtttcaaatgacaaaatctcattctttttttatggctgaatggtgcgccattgtgtttaagtaccatgttttctttatccattcgtctgttgatggacacttagattgcttccaaaccttggctattgtgaacagtgctgcaccaaacataggagtgcaaatatctctttcataaactgatgtcctttcttttgggtatatactcagctgtgggattgctggatcacatggtagctctgtttttagtttttttgaggaacctacaaactgttctccatagtggttgtactaatttacactcatgagatatttttaatggttacattattttagtatacagattgtcataggttagaattaacttaaaatttgcatttctaaaagctatcttttaaaatgtattgaactggcttgagttggtttgaaatgaccattcccttcttactaataagcttatgcagattcattcacctttcttttctcttttctgtttttctacagagaaagagaattgggcttatcccgccagaaaagcacagaagccacagtgcttcttgttgaaagacagaccctcataatgttacctctagtagaaagatagcatatgcttgttttgcttgtttgggttcatcatactctattgcttgggcagaagagcacatatgaagagaagagaaatgggaaatggggaagacaacgcagagcaccatatcttggggtgtatatagaagctacaggacaagtgtattttttatcattgcatggggagcattgacataatttctactgcagctgagcattttttaatatggataataggattctgcaagtgatacatttggtcagagaacttaataaactagtcaagtgggataggtcctgtgacagaattgtgtgatacaggtcaaacaggagttgggttatggggaaaatgccagttgaaatatgttttgatctttggagaaacctattttttcatttaacctgttctttaaatccagtatgttccagaacatacaaaaatgtttaaatgttccatttgtaagaggatatcatgtattttatatcaatttaaatgcagttatcctaatcatttttctttcatttttaccctttattaactcttcatttgtttacaaaacaaatccactctatgaacgcaatctctaattatgtgttttctttcaggtatccaaaccttgaaacctttgctctagatgtcaggcttgtttttgacaactgtgaaacatttaatgaagatgattctgatataggcagagctggccacaatatgaggaagtattttgaaaaaaagtggacagatactttcaaagtgagctgaagttataataatctctttatttttttccttctaaacaaggacaaatgagaccagcaatgtgaactgtatttacataaacgtgcaaggcacatacataatgactttctttttccttaagtataaaaaaaaagtatcagaagaatgataccatttttaaaggcttcattcctacaacaaccaaggccctcggttattggtttgtgtgatttatcagctaatttaggtagaacagggaagcacacccaaagaattttcaaaggaaagggtgttatagtgcaatagcaattaaaatatatcaaatcgcactgaatactcaacaccagagctctaatgtgggaaatggttctcctttccctctcaataaatatctatttttcatttttttactttgtagtttattttttagtgaatgtatttaattttatgaattatttatgattaaaccacatccagaatcttcgttttctgtgaaaaggaagaactagaaaattgctttaaatcttgaaaatacaaggaatgttttaaaatataaaacaaagccaagttaaactgtttacactgatgtgctataaaagcaccaaaaagaaactttactgtagagttacaagtacatttatatatatatgttgctgcatcacttgtgtagttaaattgtatttcaaaacagtgaaaaaattgacatgtatatactgttcattcttgtttatattaagtcttgttttaaatatgtattatgtgtatatattgtttgcagacattattgttcatgccttagaggattgtagcattttattttcgtctgaaggtaatgatagctatacagtctgtacagtaattatcctctaccaacactgtggcgtctccttaatcttggtagtgcctgcctttgaaacagggtgtaggggatattagttttccatttttctattttgttatataattttaagccaccagggcctaaattaaagtataatcatttgtatccatgtggaataaaattgtgacaatttcctacgcacacagtattttttcatagaaacatttccctcccatttgccttgcctcagaaataaatttaaaagacgtttgtaaccactgtgttttatctactgtgtgttgtggtggcctgttggaggcaaatagatcagattttttttgtacctacgtaagagtacttgaagttttatttaaaataaaatgttgtggaaaaggtagcattctttttttaggagtgttatttttcactatgtgtggcacggatacaataaaagacttttacaaactaaaaaaaaaaaaaaaaaaaaaaaaaa

SEQ ID NO: 170 - protein, 116 aa, Homo sapiens [sequence omitted]

SEQ ID NO: 171 - DNA, 1127 bp, Homo sapiens
gccagcagttgtgccagcagcatgcatccagcaggggtgctaggcaaagaaaaggtgggttgaggacagggcgtgttctccgggactctgtgagccacgaggaggctgcaacaccagccccagagcctccgggaaatggggacttccaggacccagccacagccccggggtggggacactgacccggagctcacgagataaaggccgtcttgcgggtttatgatatcctgggtaacggcagccttggggctgtttttcaagaggcttgctcttttagaacagcatatggaacatgtgcaatgtctagctgggacttgtttccaaatcctcttttttggagcgggagtgggtggtgaagatcagaggaaccacactggataatgactggagccgagtgatgggcacgtggggctgatttaagccaatctccacttcggggtatatttgaaattttccataacaaaagtttaaggaaagccacaagcacggatggaattccaacgtttccatttccttgatgctcttcggcactttctacacagggtctggtcagcatgtgatcctctggatactagagtttgtttttattttttattacactgttaatatgctactaatataaaatatacattaattatattaatgtaaatatcataaatatactttccttatgaaaaattatgccacaaaaacatagaggagaaaagcacctaagtctgtctttacaagagaaaatgtaagatcagaaatgtggactttctcattaaaatggaaatcagtcagatttgcagccaaagaaagtgtctcacatttttaaatcactccgtacttgcggccagagggaagaactgtcataattctcctggcagcaatgagatctgctctcttaattctgtgctgggaaatcttctggcgtgaatgggtttgaggcttgaaacattacttatataagtcacctaaactacaaaacaatgattggagtaaaaattaatgccatttttattttaagtatcagataccagatgatgcatgtcaactaagcttttagcttttcttaaaagatgcaaaggtttcaaagccaaaaatatttaataaaaataaacccaacataaaaaaaaaaaaaaaaa

SEQ ID NO: 172 - protein, 53 aa, Homo sapiens
MISWVTAALGLFFKRLALLEQHMEHVQCLAGTCFQILFFGAGVGGEDQRNHTG

SEQ ID NO: 173 - DNA, 1931 bp, Mus musculus
gggtgcgcttctcggcggggccgggcagggccgtcgcctggcggtgaggacgcgctcccggggcgggcgctatggccaccaactagggcggccggagaagcggccgaagcccaagatgccggagcgacggcacggctgcgcctccgccatcgtaggtgccgatccccttgccacagtcgagtctccatggcctgaccgtgtcttgacaataattttgagcaaaatctatgtctaataagaagataaccacatcaagatggttgggaagctgaagcagaacttactcttggcgtgtctggtgattagttctgtgaccgtgttttacctgggccagcatgccatggagtgccatcaccgaatagaggaacgtagccagccagcccgactggagaaccccaaggcgactgtgcgagctggcctcgacatcaaagccaacaaaacattcacctatcacaaagatatgcctttaatattcatcgggggtgtgcctcggagcggcaccacactcatgagggctatgctggacgcacatcctgacatccgctgtggagaggaaaccagggtcatccctcgaatcctggccctgaagcagatgtggtcccggtccagtaaagagaagatccgcttggatgaggcgggtgtcacagatgaagtgctagattctgccatgcaagccttccttctggaggtcattgttaaacatggggagccggcaccttatttatgtaacaaagatccgtttgccctgaaatccttgacttaccttgctaggttatttcccaatgccaaatttctcctgatggtccgagatggccgggcgtcagtacattcaatgatttctcggaaagttactatagctggctttgacctgaacagctaccgggactgtctgaccaagtggaaccgggccatagaaaccatgtacaaccagtgtatggaagttggttataagaaatgcatgttggttcactatgaacagctcgtcttacaccctgaacggtggatgagaacgctcttaaagttcctccatattccatggaaccattccgttttgcaccatgaagaaatgatcgggaaagctgggggagtttctctgtcaaaggtggaaagatcaacagaccaagtcatcaaacccgtcaacgtgggggcgctatcgaagtgggttgggaagatacccccggacgtcttacaagacatggccgtgattgcacccatgctcgccaagcttggatatgacccatacgccaatcctcctaactacggaaaacctgaccccaagatccttgaaaacaccaggagggtctataaaggagaatttcagctccctgactttctgaaagaaaaaccccagacggagcaagtggagtaactgagcccgtaacttcccacagggacgactgctgccttgtctacagaagggaaatctcgggaacggctgtctgctgcgacaaggagtgtctgtgcccatcgctcctgttcacctgccagcctcctgtccccagggggggtgtcacacacccgggcctccccaagtgatggctcttgagcccaggaacatgcatggccctcaggatgaggagcccagcagggacacagttctgtcacagctcctcttgtccttgtctttccttcccaggttccagtctttaatttcaaggaaaggagagtttgaagttggcattctgttaacaaaatcaggcagtctcattccgaataggttctatgtacacgttccgatgttttgtagaacactcgtgcctgttgaaacgtatcgatgtggataatagtaaataccttaattatttaaataattcattgtattgtttcagagacgtttggaaattactgtatacatttacaacctaatgacttttgtattttatttttcaaaataaaagcttaaatgtgaagcactcaaaaaaaaaaaaaaa

SEQ ID NO: 174 - protein, 370 aa, Mus musculus
MVGKLKQNLLLACLVISSVTVFYLGQHAMECHHRIEERSQPARLENPKATVRAGLDIKANKTFTYHKDMPLIFIGGVPRSGTTLMRAMLDAHPDIRCGEETRVIPRILALKQMWSRSSKEKIRLDEAGVTDEVLDSAMQAFLLEVIVKHGEPAPYLCNKDPFALKSLTYLARLFPNAKFLLMVRDGRASVHSMISRKVTIAGFDLNSYRDCLTKWNRAIETMYNQCMEVGYKKCMLVHYEQLVLHPERWMRTLLKFLHIPWNHSVLHHEEMIGKAGGVSLSKVERSTDQVIKPVNVGALSKWVGKIPPDVLQDMAVIAPMLAKLGYDPYANPPNYGKPDPKILENTRRVYKGEFQLPDFLKEKPQTEQVE

SEQ ID NO: 175 - DNA, 1342 bp, Mus musculus
ctttgcgaaagcatggcggccactttgtgagtgtccgcaagacggttaaatagggcattttaattcaagagaggaataagaaaagtggtacctaacatctcttatagattattttcatcttgatatccgcctaacacaatgcttaatatccgcctgagtgtgtggattacaaacctttagaagttgcggtcctgaagcctgagtttttgtaaacatgatttgtgcttttctacgagttgtacagcatgcggagaagctgcacgggtccttgggcaggcagttgttgccacattttgtgttcaccaaagcttgctttaaaactcagcctttgagatgggggctgcgagagcagaagataacagtacaacctagaactgtccttaggttcacccagaaaactttctggacacaaggaccggacccccggaaagcaaaggaggatagtaccaagcaagtgtctattcgcagaaatcaaagagaggaaaccggcgtctcaatgtctcagaaagtgagagaagctgggagagacgtcagctacttgatagtggtgctctttggagtcggccttacaggcggcttgttatacgcgatcttcaaagaactgtttttttcgtccagccctaatatcatatatgggaaagccttaggaaaatgcagaacacaccctgaggtgattggtgtatttggtgagcctttgaaaggctacggggaaatgtcgagacgtgggcggagacagcatgtccgtttttctgaatatgtaaacaatgggctaaagcgcatccgtgtgaaattctacattgagggctctgaaccagggaagcaaggaacggtacatgcagaagtggaagagaacccagggagtggccagtttgaatttcgatacatatttgtggaagtgactcctacaagatctattattgttgaagataaccgttccgaacaaagttaaaagcctcaagccagcgtactgcttcctgccaagagtgaacggatctgttctcactgtgaccctcaattctcttccagaatgttcagaacaagagtaaagtgcgtcacacagaaaggaagtgcagtgtgagtgttcatgagtttggatcaactgaggtctttcttacaaaaaaaatcatgaaatgtagcaacttttacattatattcaattgccaatcatgacagtatttcagctctaccttcatgataaacttttgttactaaaatttatatacacacataagtaaagttttaagtattttatgctgtttaatgttgtgtggcttttatacaatctacagaataaagaaaactaactagaagatgttaaaagcaaaaaaaaaaaaaaaaaaaa

SEQ ID NO: 176 - protein, 244 aa, Mus musculus
MICAFLRVVQHAEKLHGSLGRQLLPHFVFTKACFKTQPLRWGLREQKITVQPRTVLRFTQKTFWTQGPDPRKAKEDSTKQVSIRRNQREETGVSMSQKVREAGRDVSYLIVVLFGVGLTGGLLYAIFKELFFSSSPNIIYGKALGKCRTHPEVIGVFGEPLKGYGEMSRRGRRQHVRFSEYVNNGLKRIRVKFYIEGSEPGKQGTVHAEVEENPGSGQFEFRYIFVEVTPTRSIIVEDNRSEQS

SEQ ID NO: 177 - DNA, 3423 bp, Homo sapiens
tgttaggcaaatacacattaataagaatgcctagaagaggactgattcttcacacccggacccactggttgctgttgggccttgctttgctctgcagtttggtattatttatgtacctcctggaatgtgccccccagactgatggaaatgcatctcttcctggtgttgttggggaaaattatggtaaagagtattatcaagccctcctacaggaacaagaagaacattatcagaccagggcaaccagtctgaaacgccaaattgcccaactaaaacaagaattacaagaaatgagtgagaagatgcggtcactgcaagaaagaaggaatgtaggggctaatggcataggctatcagagcaacaaagagcaagcacctagtgatcttttagagtttcttcattcccaaattgacaaagctgaagttagcataggggccaaactacccagtgagtatggggtcattccctttgaaagttttaccttaatgaaagtatttcaattggaaatgggtctcactcgccatcctgaagaaaagccagttagaaaagacaaacgagatgaattggtggaagttattgaagcgggcttggaggtcattaataatcctgatgaagatgatgaacaagaagatgaggagggtccccttggagagaaactgatatttaatgaaaatgacttcgtagaaggttattatcgcactgagagagataagggcacacagtatgaactcttttttaagaaagcagaccttacggaatatagacttgtgaccctcttccgcccttttggacctctcatgaaagtgaagagtgagatgattgacatcactagatcaattattaatatcattgtgccacttgctgaaagaactgaagcatttgtacaatttatgcagaacttcagggatgtttgtattcatcaagacaagaagattcatctcacagtggtgtattttggtaaagaaggactgtctaaagtcaattctatcctagaatctgtcaccagtgagtctaattttcacaattacaccttggtctcattgaatgaagaatttaatcgtggacgaggactaattgtgggtgcccgagcttgggacaagggagaggtcttgatgtttttctgtgatgttgatatctatttctcagccgaattccttaacagctgccggttaaatgctgagccaggtaaaaaggtgttttaccctgtggtgttcagtctttacaatcctgccattgtttatgccaaccagaaattgccaccacctgtggagcagcagctggttcacaaaaaggattctggcttttggcaaaattttggctttggaatgacttgtcagtatcgtccaaatttcctgaccattggtggatttgacatggaattgaaaggttggggtggaaaaaattttcatctttatcaaaaatacttacatggtgacctcattgtgattcggactccggttcctggtcttttccacctctggcatgaaaagcgctgtgctgatgagctgacccccgagcagtaccgcatgtgcatccagtctaaagccatgaatgaggcctctcactcccacctgggaatgctggtcttcagggaggaaatagagacgcatcttcataaacaggcatacaggacaaacagtgaagctgttggttgaaatcataattaatgcgttactgtatgaaccacaaaacagcactatttatttagccttacttctacttccagatgcagtgcctcttttggagaagacatgtttatttttcatgttctttctgacattactttagcaattcaacttgatgtgagaagaaaaaacaaatgtttcaacacaaaatctctgttttgtgagaatactgcactatggaataattgacaaattgaaatctcatatttgtcccaaaagttgttttgagttagttctacctggtgcccatgttctgattgtgtgtgggattgcatggtgtcctgattgcatctaggtggaccggatggaatgtgctgggccactgttgggtggagagcagcacattcttacagaggagatggagcgttatgagcatagtatgtggataggtatcttcacctgcccgcccctgagtcagcctccttgacttgatagcttgaagaatccttttccactgaaatagaggataattaattgacacatctgaaatccccaatcaatcaatcaagagaaaggtagaactaaaaactccttaacttactgttgcttacacccctgaaagtctgtttttaagcaaatgggtaatagtagaaaataggttagaatctatggcttgattaaaaatatgttattacattatcatgttcaggattaggattagtagtcagttgctgtaaactattttgaacaaacagaaaagaacacggaaacatttttaacagagcatttaattatgttggaatacaggatcctagctctgtctgggaacattagtttatttgagccagctctatcagggtcttcccatggtggttcagaatagatgagcatagcatggttttgtttgtttttgctttcaattttctaatttggcatggatccatatgtatttactatcctttttctaatatattaatatatgctacatttgtatttgcattactataatactttgagttgaaaaagagtttcattgtggagagaaaaagcaaatggtatgccacaagatcactctgatttgagaaaagggaggaggggaagatagtctgaatggaaatctgaaatacggaatgttttagagaaatatgtcacttgcatatagaatgttttaattgaggtataaattaatgagacaaagtgaaaaagaaattatattcagataggactgcactacattatttgtcacacatggatctgttaccatcaggtcaattcctagtatgcataaattttttaacccttttaaaagagacctatgttgaaaacccctgaaaattcactgaagaaaaatcattactctttttctcagtaaatcatatcatctgaaatattacaaatttcaaatttctaggtgctatattaattcaatattacaataactcttacctaattattcttacaagttttaagttgtggtagtttagtgatttttttaaaagatgtgtgaaatgttctctgcaaaataattcaggccactgtctccttttatatattattataattatttattatgaagaccagtgaattacgatatttaaagtgagagaacttaattatttgcaaaggtaagttacagcttgttttttgagagaatcaaatgagtttacttttgttcttgttgtttttaactagctttaagtttaaagatggaagctaagcaatggaaatgctatacgtttttgacatttattaaatggtaccaataaagtattttattaccaaaaaaaaaaaaaaaaaa

SEQ ID NO: 178 - protein, 542 aa, Homo sapiens
MPRRGLILHTRTHWLLLGLALLCSLVLFMYLLECAPQTDGNASLPGVVGENYGKEYYQALLQEQEEHYQTRATSLKRQIAQLKQELQEMSEKMRSLQERRNVGANGIGYQSNKEQAPSDLLEFLHSQIDKAEVSIGAKLPSEYGVIPFESFTLMKVFQLEMGLTRHPEEKPVRKDKRDELVEVIEAGLEVINNPDEDDEQEDEEGPLGEKLIFNENDFVEGYYRTERDKGTQYELFFKKADLTEYRLVTLFRPFGPLMKVKSEMIDITRSIINIIVPLAERTEAFVQFMQNFRDVCIHQDKKIHLTVVYFGKEGLSKVNSILESVTSESNFHNYTLVSLNEEFNRGRGLIVGARAWDKGEVLMFFCDVDIYFSAEFLNSCRLNAEPGKKVFYPVVFSLYNPAIVYANQKLPPPVEQQLVHKKDSGFWQNFGFGMTCQYRPNFLTIGGFDMELKGWGGKNFHLYQKYLHGDLIVIRTPVPGLFHLWHEKRCADELTPEQYRMCIQSKAMNEASHSHLGMLVFREEIETHLHKQAYRTNSEAVG

SEQ ID NO: 179 - DNA, 3803 bp, Homo sapiens
gagaactcggtttggtagacttggacatctctctggcttctgaagagcctgaagctggcctggaccattcctgtccctttgttaccatactgtctctggagtgatggtgtccttccctgccccaccacgcatgctcagtgccttttggtttcaccttccctcgacttgacccttccctcccccagcgtcagtttcactccctcttggtttttatcaaatttgccatgacatttcatctgggtggtctgaatattaaagctcttcatttctggagatggggcagcaggtggctcttctgctggggctgacttgtccagaaggggacaaagtgcaatacagagccttccctaccctgacgcctcccagtcatcatctccagaactcccagcggggctccctgagctctcaaggagatgctgccatcactgggaggctcagaggacccttcctgcccaccttcggagacggcttctggaggaacggcttggccagaagacagggtgtgagtgagacagtggggcacaggttgggtttgccaaacgcctaattaccaggccaggaagcatgccaacaaagccacacgggtgtcctagccagcttcccttcacctggtgtcttgagtagggcgtctcctgtaattactgccttgccattctgcccctggacccttctctccggaccagggaggcgtccctccctaggagccacacattatactccaagtccctgccgggctccgcctttcccccaccctggctctcagggtgacgccacccacagagatttaatgagcgtgggcctggaccttccccagatgctgccaggcagcccctccccaagcctcaaagaagcatttgctgaggatggagaggcaggggagggaggcgggaggccgtcactggagtggcgtctgcagcagctgctgccccagcacccgctcagcctgtcctggctgctcacctccccgcagggcaccgggcctttcctgccctctgtggtcatctgccacctgctggatcaagtgctttctcttttacactcccctgtccccaccccagtgcactcttctggcccaggcggcaagcaagctgtgaacagctggcctgagctgtcgctgtggcttgtggctcatgcgccattcctggttgtctgttgaatctttctggctgctggagttggagataggatgttttgcttcccactgcaggagagctgccccctttcacggggttggggaagggtccccctggcctccagcaggagcacagctcagcagggtccctgctgcccacccctctgagccttttctccccagggtatggctcctgctgagtttcttgtccagcagggccttgacaggaatccagggagtagctcctggccagaaccagcctctgcggggcttgtgctctgcaaagactctgctgctggggattcagctctagaggtcacagtatcctcgtttgaaagataattaagatcccccgtggagaaagcagtgacacattcacacagctgttccctcgcatgttatttcatgaacatgacctgttttcgtgcactagacacacagagtggaacagccgtatgcttaaagtacatgggccagtgggactggaagtgacctgtacaagtgatgcagaaaggagggtttcaaagaaaaaggattttgtttaaaatactttaaaaatgttatttcctgcatcccttggctgtgatgcccctctcccgatttcccaggggctctgggagggacccttctaagaagattgggcagttgggtttctggcttgagatgaatccaagcagcagaatgagccaggagtagcaggagatgggcaaagaaaactggggtgcactcagctctcacaggggtaatcatctcaagtggtatttgtagccaagtgggagctattttcttttttgtgcatatagatatttcttaaatgaagctgctttcttgtcttttatttctaaaagcccccttataccccactttgtgcagcaaagatccccgtgcaggtcacagcctgatttgtggccaggctggacaaattcctgaggcacaacttggcttcagttcagatttcaagctgtgttggtgttgggaccagcagaaggcaaacgtccagccaacacacaggactgtaagaggactctgagctacgtgccctgtgaagacccccaggctttgtcataggaggtcgttcagcttccccaaagtcagaggtgatttgatttggggaagactgaatattcacacctaagtcgtgagcatatcctgagttttacttccttatggcttgccctccaagttctctctctcatacacacacacacccttgctccagaatcaccagacacctccatggctccagctatgggaacagctgcattggggctgcctttctgtttggcttaggaacttctgtgcttcttgtggctccactcgcgaggcagctcggaggtgtggactccgattgggctgcaggcagctctgggacggcacagggcgggcgctctgatcagctcgtgtaaaacacaccgtcttcttggcctcctggccagtctttctgcgaatagtcctctccctggccagttgaatgggggaagctgctggcacaggaaggagaggcgatcccggctgaggcttaggaaattgctggagccggctccaagcagataattcactggggaggttttcagagtcaaacatcattctgcctgtgttgggggccaggtgtgtcacacaagcatctcaaagtcaaaagccatctggggctgctgcttctctttctcaggctctggggaaaggaatctccctctcctctcacttgattccaagtgtggttgaattgtctggagcactgggactttttttctcttttccttgatggaccaacagtgcaaatgcaatctcgccatttaactttcaggtcgatttcctttcctgatcagacatctttgtgccccctttaggaaggaaaagaatacacctacgatgtgccaggcactgtgttaggcgcttttatatagatcctcgttaggatgagactaagggatgaggacatctctttataaaaggcccctaagtaatggataaacagaaacacttagaggtgagaaggtctgtcttcaagatccaaggtaagattgccttcagtctgatgtttgttctcaaggacttatcccctacaatattctcccactccatacttctccttctaccccaccatgtgctcccgtgcactcctcagatggtcagaggggtaacccaagtccttagagaatttggggaccaatagaatatgtgatgtgtgaattttctttaaaaaacttaaggagtctttgctaccttctgcttgttgagttgttttggcattcatattaaaagccagcatctcactatttattgacaggttgggctgtgtgtgtgcgcatgtgtgtatacatttccaggcgtgcctgtgtcctgtagctttttaaaaggaaacccagtcatcccactatgaatctggcatcttcttatgcttctagtgttttggccatacatcaaccaaggggtttaatttatccaatgcttgacgacatgttcaggaggggctggatcaaattttgagagggttatgggaaagggagggggagaagaaattgacatttattttattatttattttaaatgtttacatcttctttatgttgtatcaagcctgaatagaaactgatagcattaaaatactccgttcctctctctaaaaaaaaaaaaaaaaaaaaaaaaaaaaaaaaaaaaaaaaaaaaaaaaa

SEQ ID NO: 180 - protein, 122 aa, Homo sapiens [sequence omitted]

SEQ ID NO: 181 - DNA, 1684 bp, Homo sapiens
cttaggtagatggtgcaaccagtggttccagatggagaaaaggtgaaaacaagttggcatttttttgtgcccttcaagatctgacttgctttattttttaatttttatgtcttctagcacatttgaaagtgtgaacatttaaactcttattctgtttcagtttgcatatgaagatgttttaagtaagttctggaattatataaaaaaaaaatagagagagtgaggatgcccagatgacaacaagcagaaaaattcatcctttaaataaaagcctctattctcatttggaaagcaaaatgttctctcttaaaagtagcagctgtaaaaaaagcaggaaggcagaccacactaatctaagttgtaaaatatgttttggtagcttaacagagatttagctgtttctgagaaaaaaaatcaaatctaattttaaaatgaaggtatttaaaaccatggcacaagggagccttatttatggagctggtgggaagccaggatgtttccaatccgctgctcttacaggagcctgtgcctcgccagttctgtgctgcagtgggcagccaactgaagtgcatgagtcaaatgcacgaagcagcagacacctgtcctttcagaaggcaagaggtgatgaaatgagtgaattccagaactagtggaaagaaaacgtaatgattaccccagatttttttcttctatttatttatttttattgatgcataatagctgcacatagttttcgggtacgcatgataatttaatacattcataaaccttgtaaagatcaaatcagtgtacctgagatttccatcaccttaaagatttatcttctttttatactagaaccattcaaattcttctcttctagctattttgcagtatatattattataaaccatagtcaccatactgatctaacactgtctcttttttctatcacatttcagattttgtttttcttaaaattgatctgtacaaactaatatttctttctaaaaacaaggtgaaaagtgtttgggtttttttccccctacatggaattcaagctttgaacttgtgtgtttctttcaatgtcatacatactgatctttatagaatactgaaggtgctgtttcttttctaaatggtatgcctgatctttgaatgaaaggtatatcatggtgccaaaaaccttcaacatttagccttgggaccccattgcttgcacagtggacctattttctaatttggaaaatataagcctgtataccaagaatatattttttgaggctggggagtatatttttttggataagttcactaattaccataataaatagtaaatacaaattttcttgtgatctagtacataagctgggccctgtggctcatgcctgtaatctcagcattttgggaggccaaagttgatggattactggaggccaggagttcaagaccagcctggccaacatggagaaacctcatctatattaaaaatacaaaaattagctgggtgcagtggtgcacgcctgtagtcccaattgcaattgctagggaggctgaggtataagaaatgagaattgcttgagatgttgtaagttttaagcttgggcccaggaggcagagagtgtagtgagccgagattaccccactgtactccagcctgggcgacagagtgagactccatctcaaaaaaaaaaaaaaaaaaaaa

SEQ ID NO: 182 - protein, 72 aa, Homo sapiens [sequence omitted]

SEQ ID NO: 183 - DNA, 1915 bp, Homo sapiens
catcgcccctcttcctccaggtcccccttccccgcaacttcccacgagtgccaggtgccgcgagcgccgagttccgcgcattggaaagaagcgaccgcggcggctggaaccctgattgctgtccttcaacgtgttcattatgaagttattagtaatacttttgttttctggacttataactggttttagaagtgactcttcctctagtttgccacctaagttactactagtatcctttgatggcttcagagctgattatctgaagaactatgaatttcctcatctccagaattttatcaaagaaggtgttttggtagagcatgttaaaaatgtttttatcacaaaaacatttccaaaccactacagtattgtgacaggcttgtatgaagaaagccatggcattgtggctaattccatgtatgatgcagtcacaaagaaacacttttctgactctaatgacaaggatcctttttggtggaatgaggcagtacctatttgggtgaccaatcagcttcaggaaaacagatcaagtgctgctgctatgtggcctggtactgatgtacccattcacgataccatctcttcctattttatgaattacaactcctcagtgtcatttgaggaaagactaaataatattactatgtggctaaacaattcgaacccaccagtcacctttgcaacactatattgggaagaaccagatgcaagtggccacaaatacggacctgaagataaagaaaacatgagcagagtgttgaaaaaaatagatgatcttatcggtgacttagtccaaagactcaagatgttagggctatgggaaaatcttaatgtgatcattacaagtgatcatgggatgacccagtgttctcaggacagactgataaacctggattcctgcatcgatcattcatactacactcttatagatttgagcccagttgctgcaatacttcccaaaataaatagaacagaggtttataacaaactgaaaaactgtagccctcatatgaatgtttatctcaaagaagacattcctaacagattttattaccaacataatgatcgaattcagcccattattttggttgccgatgaaggctggacaattgtgctaaatgaatcatcacaaaaattaggtgaccatggttatgataattctttgcctagtatgcatccatttctagctgcccacggacctgcatttcacaaaggctacaagcatagcacaattaacattgtggatatttatccaatgatgtgccacatcctgggattaaaaccacatcccaataatgggacctttggtcatactaagtgcttgttagttgaccagtggtgcattaatctcccagaagccatcgcgattgttatcggttcactcttggtgttaaccatgctaacatgcctcataataatcatgcagaatagactttctgtacctcgtccattttctcgacttcagctacaagaagatgatgatgatcctttaattgggtgacatgtgctagggcttatacaaagtgtctttgattaatcacaaaactaagaatacatccaaagaatagtgttgtaactatgaaaaagaatactttgaaagacaaagaacttagactaagcatgttaaaattattactttgttttccttgtgttttgtttcggtgcatttgctaataagataacgctgaccatagtaaaattgttagtaaatcattaggtaacatcttgtggtaggaaatcattaggtaatatcaatcctaactagaaatactaaaaatggcttttgagaaaaatacttcctctgcttgtattttgcgatgaagatgtgatacatctttaaatgaaaatataccaaaatttagtaggcatgtttttctaataaatttatatatttgtaaagaaaaaaaaaaaaaaa

SEQ ID NO: 184 - protein, 453 aa, Homo sapiens
MKLLVILLFSGLITGFRSDSSSSLPPKLLLVSFDGFRADYLKNYEFPHLQNFIKEGVLVEHVKNVFITKTFPNHYSIVTGLYEESHGIVANSMYDAVTKKHFSDSNDKDPFWWNEAVPIWVTNQLQENRSSAAAMWPGTDVPIHDTISSYFMNYNSSVSFEERLNNITMWLNNSNPPVTFATLYWEEPDASGHKYGPEDKENMSRVLKKIDDLIGDLVQRLKMLGLWENLNVIITSDHGMTQCSQDRLINLDSCIDHSYYTLIDLSPVAAILPKINRTEVYNKLKNCSPHMNVYLKEDIPNRFYYQHNDRIQPIILVADEGWTIVLNESSQKLGDHGYDNSLPSMHPFLAAHGPAFHKGYKHSTINIVDIYPMMCHILGLKPHPNNGTFGHTKCLLVDQWCINLPEAIAIVIGSLLVLTMLTCLIIIMQNRLSVPRPFSRLQLQEDDDDPLIG

SEQ ID NO: 185 - DNA, 1101 bp, Homo sapiens
ccgagatctcgccactgcactccagcctgggtgaaaagggaaagaaaaccaacaagccaggctgattttctagagggatcagtgatgtggggtagaatgacaccttccctgtggcttgttatgcctccggttttgtttttgaatcttggttgctggtggggtattgccccctcggctcctctatgctttcgcgtgtgtgaaaatgcaggagtggaccactgtgcacagcaggaccatggctgtgagcagctgtgtctgaacacggaggattccttcgtctgccagtgctcagaaggcttcctcatcaacgaggacctcaagacctgctcccgggtggattactgcctgctgagtgaccatggttgtgaatactcctgtgtcaacatggacagatcctttgcctgtcagtgtcctgagggacacgtgctccgcagcgatgggaagacgtgtgcaagtaagtgtctgaaggacaagcaggacctgcacaggtgttccgtgggtgccggtgtgggtgcgctgccgacgtgtatatgtgcctgtgtgtcctgtctccaggctttgctgggcctgcatgaatgtgtgtgacagcatctcttagccattgagccttggtggctgcttcaccaactcccctcaatcttccttcccccgtgccctgaagtggctttttgctgtttccacctccctgtcattctgaagctggacagagcccttgtcctcagctctctctgctctacccccagccagctagtatggcacatgaataataataatgtggacccctcactgactgctagctgcatgctgggcactgtgctaagcacacatgctgttgcattcaggggacagatgaggaaaaactgtagctcagattaagtaaagagcctaaggtcaccagctagtgagtggtgggttcaaactctgctggctgtgattccaaagtcagtcctgggagaagaggatgctcctgagaaacgctttgccctgctttctgaccccggtgatctcacagcacatggtgaggctggcagtgatgtgtcctgggtacatatttccttgcagctggtcccaaatcttgtctcaattaaaaaaaaaaaaaaa

SEQ ID NO: 186 - protein, 166 aa, Homo sapiens
MWGRMTPSLWLVMPPVLFLNLGCWWGIAPSAPLCFRVCENAGVDHCAQQDHGCEQLCLNTEDSFVCQCSEGFLINEDLKTCSRVDYCLLSDHGCEYSCVNMDRSFACQCPEGHVLRSDGKTCASKCLKDKQDLHRCSVGAGVGALPTCICACVSCLQALLGLHECV

SEQ ID NO: 187 - DNA, 1604 bp, Homo sapiens
aggggcgagctggctggactcggagcgcggtcgaggctttctgcgttcgcggcggcggaatggcccgtgcgcggctcgccgcgtcgcggctctgtggtccctagacgtcggctcccgccctcggcgctgatctccggcgcgggcactgctttccactcggctcctgtcgtccgttctctcaggctcccgttcaggatttttagactctgaggagcagttggagctaatccacattatggaaatggaaaccaccgaacctgagccagactgtgtagtgcagcctccctctcctcctgatgacttttcatgccaaatgagactctctgagaagatcactccattgaagacttgttttaagaaaaaggatcagaaaagattgggaactggaaccctgaggtctttgaggccaatattaaacactcttctagaatctggctcacttgatggggtttttagatctaggaaccagagtacagatgagaacagcttacatgaacctatgatgaagaaagccatggaaatcaattcatcatgcccaccagcagaaaataatatgtctgttctgattcctgataggacaaatgttggggaccagataccggaagcccatccttccactgaagctccagaacgagtggttccaatccaagatcacagctttccatcagaaaccctcagtgggacggtggcagattccacaccagctcacttccagactgatcttttgccagtttcaagtgatgttcctactagtcctgactgcttagacaaagtcatagattatgttccaggcattttccaagaaaacagttttacaatccaatacattctggacaccagtgataagctgagtactgagctctttcaggacaaaagtgaagaggcttcccttgacctcgtgtttgagctggtgaaccagttgcagtaccacactcaccaagagaacggaattgaaatttgcatggactttctgcaaggcacttgtatttatggcagggattgtttgaagcaccacactgtcttgccatatcattggcagatcaaaaggacaactactcaaaagtggcagagtgtattcaatgattctcaggagcacttggaaagattttactgtaacccagaaaatgatagaatgagaatgaagtatggaggacaagaattttgggcagatttgaatgccatgaacgtgtatgaaacaactgaatttgaccaactacgaaggctgtccacaccaccctctagcaatgtcaactctatttaccacacagtctggaaattcttctgtagggaccactttggatggagagagtatcccgagtctgtcattcgattgattgaagaagccaactctcggggtctgaaagaggttcgatttatgatgtggaataaccactacatcctccacaattcattcttcaggagagagataaaaaggagacccctcttccgctcctgttttatactgcttccatatttacagtaagtgtcgagtatgaagttgcaatatttactctcattttatgtaaatgcattcctgaatactagagataaaaaataaataagagtctaccttggttagtacccctaaaaaaaaaaaaaaaaaa

SEQ ID NO: 188 - protein, 415 aa, Homo sapiens
MEMETTEPEPDCVVQPPSPPDDFSCQMRLSEKITPLKTCFKKKDQKRLGTGTLRSLRPILNTLLESGSLDGVFRSRNQSTDENSLHEPMMKKAMEINSSCPPAENNMSVLIPDRTNVGDQIPEAHPSTEAPERVVPIQDHSFPSETLSGTVADSTPAHFQTDLLPVSSDVPTSPDCLDKVIDYVPGIFQENSFTIQYILDTSDKLSTELFQDKSEEASLDLVFELVNQLQYHTHQENGIEICMDFLQGTCIYGRDCLKHHTVLPYHWQIKRTTTQKWQSVFNDSQEHLERFYCNPENDRMRMKYGGQEFWADLNAMNVYETTEFDQLRRLSTPPSSNVNSIYHTVWKFFCRDHFGWREYPESVIRLIEEANSRGLKEVRFMMWNNHYILHNSFFRREIKRRPLFRSCFILLPYLQ

SEQ ID NO: 189 - DNA, 3196 bp, Homo sapiens
gcgagcctgcaggggatgactgctgggtgaacctaagttacacagtggttgctttcaccaaacagaccatgggcttcttggaagaggccctgaagctgtatttcccagagctgcacatggtacttttggagagcctggtggaaatcattttggttgctgttcagcatgtggattatagtcttcgatgtgagcaggatccagagaagaaagcttttatcagacagaatgcatcctttttatatgaaacagtcctccctgtggtggagaaaaggtttgaagaaggtgtggggaaacctgccaagcaactccaagatctgaggaatgcatctagacttattcgtgtgaatcctgaaagtacaacatcagtggtctaatgcttgggtctgtttatatgtgtatatatgcagagagagagcttatatattatttatatttatattaagttgtattagcatactctatagtttcaaacacaacttgaaaattaaaagtgccctcttaaaaatacaaaaatcaaaaagaggaaaataagttaaattaagcccaagtaacaaaaatactggaattattaaaacgtatagtatgctagctatccttttaaattatgctaattctcttcttctgaaattatggtcacactatatactatagcatttcggttttatcctttgataaaacttttcttttttcttttttttttttgagacagggtctcaccccgtcgcacaggctggagtgcagtggcaaagtctcgactcactgcagccttgacctcccgggcccaggtagtcctcccacctcagcctcccatgtagcaggaaccacaggcaggcaccaccatacccagctaatttttgtattttttgtagagatggggtttcgccatgttgcccaggctagtcttttctttcttcttttttttttttccccacagtatatattatacagcagtcataatatctataaatacatagagtttatgttgtgaagtttcccagttcactgaaatgttaggtttcctaaagggtacagtgccgtataaaacaacctgcctcatatatttctcctcaaaacgttggactatttgggaaaaggaaaagagttgggaaaattggttttaaggtaagttttagtcaaaagaattctttcttgaaactagctggtttgtggattcagatactctgatccttacagaatccaagaggaagctttcataaaaacaattcagcaaatatttccaatataatttgaatggctaattttcagttgctaattaattagcagctttgtaatacttgatttgggagcatttacttggaaatcctaaggactataataaaagttttcaacatatttctaaattgtgtgagtttccagctgtagcttttgtctttgtcacattttaaaaaataataagcaagacacattggggacactggcagcagttgccaggttttagctgccaccgcttcagtatgagatatagctgtcccatcttcccccattcagggtaggagatatagtgacccagacttcatgcaaatggaaaaaaagttttaactgaaatatttatttagatttcagggtctagatggatgggaaagtagaaaaacatatgcaaatctcagtgttctcactatgaccactctgagcagagatttggttttgtttccttttgtaacaaagtgaaaacaggtgagacaatgtgcccaaaacaaagggaagaagagaaccttctgtgactcctaaaatgttccatgctgcatttttgtttcattttttatttttccttgctttgtttttaaacatgaatataatgcttacttcaaattgtttagtaaaacaaaataactaaagaaatgtgagcttcccaaggtttctaaactatcgctgttgtatattctatagcgttccttattctttgagggaaactgtgcttgctgtgatccattttgtctctagcttctagttgtgattcttgtccataagcaccaaatttgatgcccatgatttcaaaaggtcatttcttttatctgaatgaaaatggtggtactaagactgtgaaaattatgtgaaacctaaagtagttgccaaagtggctcagggttgtaaaattcattgacttaattattcatgtgccagatcaacccctttattttctctttagctgtgcatatttaaaatattggaaagtatcagatttacagattttctttgactaatttttttcacataactttaggattttcgaaagttgtaaccataactggatatcttagctgagcaaaggcggttataatttgtctttttaagatcactggaaattgataaaattttgtgataattatgattattctgtgccatttacagtttctaatactatactgtatgaaatatgtataaatatatgatgctgagtctgtggaatgatacttctgaaatcaaaattcctcataaggcatgaagttgtaaaaacttgaatgtgtatagttagatatttaaatggttgcttcttcatagaattgtctgctttttaaaactggaagtacaggattttcttcaggtaaaatctgtgtgttccaattacagttgtagctgaaggaagtatgctttggtgagtcaattagtatgggaacttgactaaagacccccagtgttgtaacgtacctttgtacccagacaaaacaattatgttacattcctcaaagtggcatgggctttcttctctaattcttctgttttattagacccaagacaagttctaaaaattgaatgcaatgagagattgtccagaaatgtaatatatactaaaatataccacttaagcattgattgccttttcttgtttgcttcaagaatataaaacttgttacttgagcttggaatcatgggcttgattgaattaattactcttggggaaaaaagacaccttgtggcattaagtcttgctttggttaaagccttatttcacataattgctaaaaactcatttttgtttaatatactacctatagtttaattatcggcacttgtattttgtaacttgatatcttacctaggattgggaatttgggacatgacatgtactataaaagtcagtctatgtacatactgcttattgatgtgctgtgatatgagggaatctgaaatgtttcataaaaataaagcttaaaaattgtcaaaaaaaaaaaaaaaa

SEQ ID NO: 190 - protein, 101 aa, Homo sapiens
MGFLEEALKLYFPELHMVLLESLVEIILVAVQHVDYSLRCEQDPEKKAFIRQNASFLYETVLPVVEKRFEEGVGKPAKQLQDLRNASRLIRVNPESTTSVV

SEQ ID NO: 191 - DNA, 1673 bp, Homo sapiens
ggagaccacagagccctgggttgtggaagaggtggctgttccctgtcatcagtatgcagcgattgctctttccgccgttgagggccttgaaggggaggcagtatctgccgctcctggctcctagggcagcgcctagagcacagtgtgattgcatcaggcgccctttgaggccagggcaatacagcaccatctctgaagtagctttgcaatctggaaggggtacagtgtcccttccctcaaaggctgctgagcgggtggtgggccgatggctcctggtctgcagtggaacagtggctggagcagttattcttggtggagtaactaggttgacagagtctggcctctcgatggtagattggcatttaataaaggagatgaagccacctacaagccaagaggaatgggaagcagaattccaaagataccagcaatttccagaatttaaaatcttgaatcatgatatgacactgacagaattcaagttcatctggtacatggagtactcacaccgaatgtggggtcgccttgtaggccttgtgaacatcctgcctgctgcctacttttggagaaagggctggctcagccgtggcatgaaaggacgtgttcttgccctctgtggcctcgtctgcttccagggtctgttgggatggtatatggtgaaaagtggactagaagaaaaatcagactcccatgacatccctcgggtcagtcagtaccgccttgctgcccacctgggatcagccctggttctttattgtgccagcttgtggacctcactgtcactgctactccctccgcacaagttgcctgaaacccaccaactcctacagttgagacgatttgctcatggaacagcaggtctggtgttccttacggccctctcaggggcttttgtggcagggctagatgctgggcttgtttataactcctttcccaaaatgggagaatcctggatcccggaggacctctttaccttctcccccatcctgaggaatgtttttgagaatcccaccatggtgcagtttgatcaccggattctgggaatcacttcagtcactgccattacagtgctctacttcctctctcggaaaattccccttcctaaaaggaccaaaatggcagcagtgactctgctggctttggcgtatacacaggtgggcttgggcatcagcacgctgctgatgtatgtcccaactcctctggccgccactcaccagtcaggctccttggctttgctcactggtgctctttggctgatgaatgaactccgaaaagtcccaaaatgattcttaaaggaccagcctcctgggactgtgactgcttttgaaagctcttcaaagatcataaaaacttgggcttttctacaaaatgaccttgacataccaagtggtttccaaatggtcaacttacttaaaaatcttttcctgttttgagatagtcactggatcaagaatgcattaagtgtggttaccctaaatgttcccttttaaatctgcttttcatgttgaaaatcagttttaatgtagagaaagaaatgtctgccatttgctgcttaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 192 - protein, 410 aa, Homo sapiens
MQRLLFPPLRALKGRQYLPLLAPRAAPRAQCDCIRRPLRPGQYSTISEVALQSGRGTVSLPSKAAERVVGRWLLVCSGTVAGAVILGGVTRLTESGLSMVDWHLIKEMKPPTSQEEWEAEFQRYQQFPEFKILNHDMTLTEFKFIWYMEYSHRMWGRLVGLVNILPAAYFWRKGWLSRGMKGRVLALCGLVCFQGLLGWYMVKSGLEEKSDSHDIPRVSQYRLAAHLGSALVLYCASLWTSLSLLLPPHKLPETHQLLQLRRFAHGTAGLVFLTALSGAFVAGLDAGLVYNSFPKMGESWIPEDLFTFSPILRNVFENPTMVQFDHRILGITSVTAITVLYFLSRKIPLPKRTKMAAVTLLALAYTQVGLGISTLLMYVPTPLAATHQSGSLALLTGALWLMNELRKVPK

SEQ ID NO: 193 - DNA, 2190 bp, Homo sapiens
taggccatgaaggccggccttcatggcctaaaatgtttcaagaacaacacattgatatgtggaaatattctataaggttttcttttgttcccttagaattcattggagggatgcagtaaaaactgtagtagaaaccttgaaacacccatatgtgaaaaggtctgtggaaattgaggcctctacattaaaagtgcagaaccaactgttttacagtcaaagtgctaggaaacctgataggatacttccctttggcacaaaaacaccctgggtgctacatacaggagcatgacctttggtgaatatgtggcactaattttttttaccttaatcatattcttgtcaagtaggcaacccattgccccttggagaccacaccagccctgtaagttctcaccagcagcatggagattaggaagaggggctgctgtgaccaggagatacacacggctttaagtaactgagagcctaaagaaagtaacccagggagtccggtccagttttaatatttgtggatttgttgtcacacacattgtttagtcctgaaactaaaacctattttataaatagtagggttaattcctcgaaacaatttctttattaataaatgtcctgtgggtttagaaatatcaggtaaatatttgaatacagaatgatgattgcaattactgttacaagcgtgaaacacaaacttcagatcaaatctagagttgcttcatttaatgcatgctagcaacagccttaactttggattcagttatttgaaacacttttccggcatctttccctttctaatgttgtggggtggaaaccggatggcaaatcactgtgagccggatacctcagcacagtccaccttgtgtgtgacttcacaaatgggggacttcacaaatggggtaactgaatgttattactttcaaattttgacatggagcattatgatcaaggaaatggagctgccttatacattaaacccgtgatttaatcctattgacattttcatagccatgcctccagattttatctttttggcaaaattctgattccacagtttggtctgattgaaataaatattccctggacgtctggctaaaaattttgctaacaatcccagaggtgccattttcttattaataaatttcattggagccttatttcttactatattcaatttcgtttcaaacctgcaagtccctgggatggtcccacgactagggcctgcacatttcttacaatggcaaagcattttttaaaatttagggtcaggttgaaaaattctaggactaattctgtagagaggagggactgttaactaacgtgagtggggacagaggagtaggttaccacatttggagcagtaatagatgcaaacgatgtaaatttgaaatttgcccctttagttaaagaaggagcctgcaaagtccatttctctgttttcagccctgtcagtcacccatttaggatgttggcaaagtactgcttgagcagaatgtgtaagaaagtaataatgaaagcaaaagtatgtcagacagttacttcttccacatggttagaggcatgtgattttcagcactgtgtgttacagaaatgtcaggaatggtgtattataacgtgtgcaagataatgtcagtgtgcacagagggtcttttttccttatctgattagtactgttaatgttcaaagaataaaaatggttttacatttagattctgagatagcaaaacctgatttttcaaccatgacctgcatgagagaagcatcctaggaagtcttagatcatacttttgagtttttaattttaatttatatagtgtttttttatgtcttaatatttttgtgaactggtgtaaattgttaatgcatataagcttgtgtatttttgtaaatagttttgtgatttatttcttgccccatatgtaaatatttagagtctcatttcttgcaaacttatttgaagctgagttgtgggtttgggttttgtttgtttctttggttgcagggtggggtggggggtggcaggggagggaggaagggatttttgtacctggagatggagatatcttgtggtttaaagcaaatgtcccactgaaagtgattcaaatatcaacagaattatttcaggttaaaacaaaaaaaaaaaaaaaaaaaaaaaaaaaaaaaaaaaaaaaaaaaaaaaaaaaaaaaaaaaaaaaa

SEQ ID NO: 194 - protein, 85 aa, Homo sapiens [sequence omitted]

SEQ ID NO: 195 - DNA, 1087 bp, Homo sapiens
cagagacagacaactggtacctctccctgcagctcatgtgccctgagaatgctgaggactgtgagcaggctgtggtccacgtggagaccaccttgtacctggtgccctgtttgaacgattgtggaccctatggccagtgcctcctgctccgcagacacagctacctgtatgccagctgcagctgcaaggcaggctggcgtgggtggagctgcacggacaacagcacagcccagacggtggcccagcagagggcggccacactgctgctcacgctcagcaacctcatgttcctggcccccatcgccgtctcagtgcggcgattcttcctggtggaggcctccgtctacgcctacaccatgttcttctccacgttctaccacgcctgcgaccagcccggggaggcggtgctgtgcatcctcagctacgacacgctgcagtactgcgacttcttgggctccggggcggccatctgggtcaccatcctgtgcatggcacggctcaagacagtcctgaaatacgtgctgtttcttctgggtacactggtcatcgccatgtccttgcagctggaccgcaggggcatgtggaacatgctggggccctgcctctttgccttcgtgatcatggcctccatgtgggcttaccgctgcgggcaccggcgccagtgctaccccacctcgtggcagcgctgggccttctacctcctgcccggcgtctctayggcctctgtgggcatcgccatctacacctccatgatgactagcgacaactacttactaacaacccacagcattcttggcaacatcctgctggccgggagcgcagccttgytgctgccgccacctgaccagcccgccgagccctggggcctgytcgcagaaattcccctgccactatcagatctgcaagaacgatcgggaggaactgtacgcagtgacgtgacactggcctggggacagctgctgctctgatgacctcttcagccaggagctgtatcgagggggaggcgcctggtccagccytggacagattgatttccagctgaataaattggcctagataccctcaaaaaaaaaaaaaaaaaaaaaaa

SEQ ID NO: 196 - protein, 310 aa, Homo sapiens
  UNSURE (228)
  UNSURE (279)
MCPENAEDCEQAVVHVETTLYLVPCLNDCGPYGQCLLLRRHSYLYASCSCKAGWRGWSCTDNSTAQTVAQQRAATLLLTLSNLMFLAPIAVSVRRFFLVEASVYAYTMFFSTFYHACDQPGEAVLCILSYDTLQYCDFLGSGAAIWVTILCMARLKTVLKYVLFLLGTLVIAMSLQLDRRGMWNMLGPCLFAFVIMASMWAYRCGHRRQCYPTSWQRWAFYLLPGVSXASVGIAIYTSMMTSDNYLLTTHSILGNILLAGSAALLLPPPDQPAEPWGLXAEIPLPLSDLQERSGGTVRSDVTLAWGQLLL

SEQ ID NO: 197 - DNA, 4252 bp, Homo sapiens
cgaacttggtcggggcgcggatcccgagagggaaagtcataacaaccgcacgagggagttcgactggcgaactggaaggccacgcctcctcccgcctgccccctcagccctgtggctgggggcagagctcagactgtcttctgaagattgatgtctatttccttgagctctttaattttgttgccaatttggataaacatggcacaaatccagcagggaggtccagatgaaaaagaaaagactaccgcactgaaagatttattatctaggatagatttggatgaactaatgaaaaaagatgaaccgcctcttgattttcctgataccctggaaggatttgaatatgcttttaatgaaaagggacagttaagacacataaaaactggggaaccatttgtttttaactaccgggaagatttacacagatggaaccagaaaagatacgaggctctaggagagatcatcacgaagtatgtatatgagctcctggaaaaggattgtaatttgaaaaaagtatctattccagtagatgccactgagagtgaaccaaagagttttatctttatgagtgaggatgctttgacaaatccacagaaactgatggttttaattcatggtagtggtgttgtcagggcagggcagtgggctagaagacttattataaatgaagatctggacagtggcacacagataccgtttattaaaagagctgtggctgaaggatatggagtaatagtactaaatcccaatgaaaactatattgaagtagaaaagccgaagatacacgtacagtcatcatctgatagttcagatgaaccagcagaaaaacgggaaagaaaagataaagtttctaaagaaacaaagaagcgacgtgatttctatgagaagtatcgtaacccccaaagagaaaaagaaatgatgcaattgtatatcagagaaaatggttctcctgaagaacatgcaatctatgtttgggatcatttcatagctcaggctgctgctgagaatgtgtttttcgttgctcacagctatggaggacttgcttttgttgaactgatgattcaacgagaagccgatgtaaaaaataaggtaactgctgtggcattgacagactctgttcacaatgtgtggcatcaagaagctggcaaaacgattcgagaatggatgagagagaactgttgtaattgggtctctagctcagaaccattagacacatcagtggagtccatgctacctgattgcccccgggtctcagcaggcaccgaccgtcacgagctaacttcctggaagagctttccgtctattttcaaattctttaccgaagcctcagaggccaagaccagctccctgaagccggctgtgacgcgccgctcccaccgcatcaagcacgaagagctgtaagaggagcgagcgcgacgggggaggccgccctggcgcacccacccgcacgcctcctcactgcttgtgtcgagcggactcccagcccctcattagattgttttcttcccagagcccagggtcgtgatatatacatctaaatagcgttttgcattatttctagatgagtgcaactgtcaaagcaatatgggttcactggtcgtgcttcctgcgggctgagcgcgggctgagcgctgccagtcagcgctcacattaaggctgacagcgccctgcctggctcggccggcgaagctctaattgccctgaaggagaccgcgcgggcgctgcgggtccggcggcgtcggcgcggtcctagcgcccgccgtagcggagccgctgctgcttccccctccttaatcatgtccatctttccccgctctcaactggagcaaacttctattccagacttcagatttccattttcactaggtttttccctgagggcatcttattacccacctctttttttttttttttaaggaagttccacacacactccaaagccgatttcaaattcagaacctgaatgccatgatgtaccatgtagtttggggaatgcaaattgttctctccctgttttcatttcgggaccagaaaaaaaaacaactctgcataaaaatctataaagtactgaatccgcattactgctgaaaatccttttgccagctaattgtgagagtttacacaagtctctgatttcagaagagactgttaaaaccaaaacaatgatgggcagctcttttaagtgtaggtgtgaaggtcccattttcaatggaatttccaactgttatcactgggattgactttactttttaaacacataatgtgtactgtgtggcattgtaatttagatataacacacttaagttttgctttttaaatctctctgtggagaaaggttgttgttaatcctttccagaatgatctattctgagctttcctggagttaggcccttctagggagagaatttttttattgtcatcaggaaacgaaacctttgtgctctgcagcctttttcttctgaaaggctaagtggggctaaaattatccttttagttttctgctttaacaagaagagaaggacttggtgtgaaagaatttcttctctcagtccaatggtataatttttagcactccgcttcctctgtrggtttagccctacagtttgaacaattattttaatccagctaagatgtggccaacactgtgaaatacaggagatgttaaggttttgctgacttttagattaaaatcttaaaatttcacattatattttaattacttgaaaagtttaattacctagtaaagattgtttctaatagatgagaacataaatttaatagttttcatccttttttaaagtgtgtgcaacaattaatatgcctgccttatttgaggacatgataaaatgtacccaaaatgacttcgaactgtctaatgcctcagaaaagtaacttaaaatcaaccacttcgtgcttaaatgtttttatattatgaaccgatttcaacaagttttaaagctgttaatatctcattgctgtgtttgacatacaagtacattcattggttctatagtcttaatgatgacaaattaaagatgttttttcagtgctcaggtatgcatatattttcgtgttacttaagcaaaatcaactatataggaggaaaaatcaaaatggcattttattcagctaccagatggcttaaaatgagtaaaccccaacaacatcaagcacatttgtctgtgatattgaccctttttatccaatcattactattttagaagtagtggtgaactgtgtaaaataatggtatcttcagcagtgttccggaatcttagttgagggacaaacattccttccagtgagagattcaagaattgatgtgaattgaatatatttttcaaggtattataaattgtgttgtgttttcagtataagaaaatgttgacaggaaacaattttatagcatttataaaaaaccaacacttgtgcaatgctaaattggcgaagcttctgcacctgaattagagcacaataaatatgctgtttataaaccaaatcactgctattccttctctgaaatcatctctcctctttttaaacctttacttatagctagaataaatcactatttaattgcctctaatactttaaaactactggatggttaggcctggtttaactatttatggagagctatttgcaaacttaagttgtgtaaatgtaatttctacttgtgaatctgagctgtagcagcccagagggagctaggcaaactattccagaccaccaactgataagtgatcagattctttgtaatgtaggattttttaacctgttgattatgggtttgttgatataaatgtaataatgttcacctcgattttcctggagtaacaaccagcgttggtagtagcccacctgcatgaggacggccagtgttaacaatatttttgttctgatcttcttccccatttttgtttcctcaaacaggtttttaggagagtggagatttaaagtcaggatgtggcctttttattttaattatatacttaattcttagaacaagtagaatgggaaaggagtgactgataaatctaagattcaaaatagtcccgtcgaaacttaaaggccagattattgctttggagctttctataggtactagccatcccgtcgttaaatgttttcatggatatttgaaaagaagaccatgtacctttaataactgttcttttctcgagtttctgcctcgtgctttgacctggattgcattattattgtttatgcgaagtaaaaaaaaaaaaaaaaa

SEQ ID NO: 198 - protein, 416 aa, Homo sapiens
MSISLSSLILLPIWINMAQIQQGGPDEKEKTTALKDLLSRIDLDELMKKDEPPLDFPDTLEGFEYAFNEKGQLRHIKTGEPFVFNYREDLHRWNQKRYEALGEIITKYVYELLEKDCNLKKVSIPVDATESEPKSFIFMSEDALTNPQKLMVLIHGSGVVRAGQWARRLIINEDLDSGTQIPFIKRAVAEGYGVIVLNPNENYIEVEKPKIHVQSSSDSSDEPAEKRERKDKVSKETKKRRDFYEKYRNPQREKEMMQLYIRENGSPEEHAIYVWDHFIAQAAAENVFFVAHSYGGLAFVELMIQREADVKNKVTAVALTDSVHNVWHQEAGKTIREWMRENCCNWVSSSEPLDTSVESMLPDCPRVSAGTDRHELTSWKSFPSIFKFFTEASEAKTSSLKPAVTRRSHRIKHEEL

SEQ ID NO: 199 - DNA, 1405 bp, Homo sapiens
gcagttaggaaagtagcgttatgagttgtactgaaaatgttgattctctaatctgccagaaaaggacctgtcttttcatgcagatttcatattgtctttgtccttttcattgcttcttgaccttcctggcaggtgtcgctcagtttcttcctgtttcccttcctgtcctctccacacctgctatcccgtcccactcccatctacctcccgggaagccagccctgcatgctgagtttgtgacctgcttcattcccatttcatttctagagggtttagaggtgacctggaaccgttccctttccctctcctaccccctcctctgcaacaccaagaggcctggaggggcagacagaaagcagccagccacggcgggaagacatgcatgtttggttgcagctggactgcgatcgtagttcctcctggagatagagtgtgaggaacttaggacactcttcctcagactctgggatcatcacataccacactgccccgctcagagtttcgtcctgagctccctaaccagctcaggtggagcagaagcctgctctcactcctccatctctggtgctcccttgggcggggacctgtccctcactcttaggcccagaacctgtccaagggacaggtagggtccaggtgccactttgggtagctggctgttggaatgcccacactggtgctgcctgtggcatagccactgctgtacgtttttggttgtttttaagaaactcgatgaagaggggtgtcattctgggctcggggtggttgccaatttttcaccagaaagggagccaccccttgcaaccacttctgtctccgttagccccccctctgccctcctccaagccaaagcgtggcctggcttttgtcttcccatttagttttcctcttttacccttccttttgtgcttaatttattaaaatagttgctgtataatttattttcataaactataaaaaaatactaaatggttaaaatagacttgcaggccaatcttaaatggggtgggaggggtctgagggtgggatggggaaagggaaagaggttttgatataaacaaaacaaatgcactttgggtgtgtttttgtatttttctggggatagagggggtggggttagggatgtccctgtagattagttccagaatggggtgtctgtatatactgtattaataggcatgtttgactctcgtaaagggacgttagtagctgctgcaggtcctgtttggaaaccccatgtacaattcccagttttttgtaagtgtcagtgcgagagacatttgactcttgtgtttgtatctcctttttatgattgctgtacctacccatgtctttttggggaggggtgaaaagagatttgaaataaaaatgtttagaaattaaaaaaaaaaaaaaaaaaaaaaaa

SEQ ID NO: 200 - protein, 130 aa, Homo sapiens [sequence omitted]

SEQ ID NO: 201 - DNA, 1055 bp, Homo sapiens
gttagacacaggacctgctgggccacagaaaggaggctctgggtagacgcactagattactggataaatcacttcaatttcccaatgaattttatattgtttatttttatacctggagttttttccttaaaaagtagcactttgaagcctactattgaagcattgcctaatgtgctacctttaaatgaagatgttaataagcaggaagaaaagaatgaagatcatactcccaattatgctcctgctaatgagaaaaatggcaattattataaagatataaaacaatatgtgttcacaacacaaaatccaaatggcactgagtctgaaatatctgtgagagccacaactgacctgaattttgctctaaaaaacgataaaactgtcaatgcaactacatatgaaaaatccaccattgaagaagaaacaactactagcgaaccctctcataaaaatattcaaagatcaaccccaaacgtgcctgcattttggacaatgttagctaaagctataaatggaacagcagtggtcatggatgataaagatcaattatttcacccaattccagagtctgatgtgaatgctacacagggagaaaatcagccagatctagaggatctgaagatcaaaataatgctgggaatctcgttgatgaccctcctcctctttgtggtcctcttggcattctgtagtgctacactgtacaaactgaggcatctgagttataaaagttgtgagagtcagtactctgtcaacccagagctggccacgatgtcttactttcatccatcagaaggtgtttcagatacatccttttccaagagtgcagagagcagcacatttttgggtaccacttcttcagatatgagaagatcaggcacaagaacatcagaatctaagataatgacggatatcatttccataggctcagataatgagatgcatgaaaacgatgagtcggttacccggtgaagaaatcaaggaacccggtgaagaaatcttattgatgaataaataactttaattattttgtcatcaaaaaaaaaaaaaaaaaaaaa

SEQ ID NO: 202 - protein, 294 aa, Homo sapiens
MNFILFIFIPGVFSLKSSTLKPTIEALPNVLPLNEDVNKQEEKNEDHTPNYAPANEKNGNYYKDIKQYVFTTQNPNGTESEISVRATTDLNFALKNDKTVNATTYEKSTIEEETTTSEPSHKNIQRSTPNVPAFWTMLAKAINGTAVVMDDKDQLFHPIPESDVNATQGENQPDLEDLKIKIMLGISLMTLLLFVVLLAFCSATLYKLRHLSYKSCESQYSVNPELATMSYFHPSEGVSDTSFSKSAESSTFLGTTSSDMRRSGTRTSESKIMTDIISIGSDNEMHENDESVTR

SEQ ID NO: 203 - DNA, 3161 bp, Homo sapiens
gacctgctgtcctcatccccagcaaacccttggcccggagatgcttccccgctatccacgcctacaagggtgtcctgatggtgggcaatgagacgacctatgaggatgggcatggctcccggaaaaacatcacagacctggtggagggcgccaagaaagccaatggagtcctagaggcgcggcaactcgccatgcgcatatttgaagattacaccgtctcttggtactggattatcataggcctggtcattgccatggcgatgagcctcctgttcatcatcctgcttcgcttcctggctggtattatggtctgggtgatgatcatcatggtgattctggtgctgggctacggaatatttcactgctacatggagtactcccgactgcgtggtgaggccggctctgatgtctctttggtggacctcggctttcagacggatttccgggtgtacctgcacttacggcagacctggttggcctttagtgagtcacagtctcccattcctgcccccacatgaggccttggagggagtggggagcccagccggctcagcctttgccctttgcagtgatcattctgagtatccttgaagtcattatcatcttgctgctcatctttctccggaagagaattctcatcgcgattgcactcatcaaagaagccagcagggctgtgggatacgtcatgtgctccttgctctacccactggtcaccttcttcttgctgtgcctctgcaccgcctactgggccagcactgctgtcttcctgtccacttccaacgaagcggtctataagatctttgatgacagcccctgcccatttactgcgaaaacctgcaacccagagaccttcccctcctccaatgagtcccgccaatgccccaatgcccgttgccagttcgccttctacggtggtgagtcgggctaccaccgggccctgctgggcctgcagatcttcaatgccttcatgttcttctggttggccaacttcgtgctggcgctgggccaggtcacgctggccggggcctttgcctcctactactgggccctgcgcaagccggacgacctgccggccttcccgctcttctctgcctttggccgggcgctcaggtaccacacaggctccctggcctttggcgcgctcatcctggccattgtgcagatcatccgtgtgatactcgagtacctggatcagcggctgaaagctgcagagaacaagtttgccaagtgcctcatgacctgtctcaaatgctgcttctggtgcctggagaagttcatcaaattccttaataggaatgcctacatcatgattgccatctacggcaccaatttctgcacctcggccaggaatgccttcttcctgctcatgagaaacatcatcagagtggctgtcctggataaagttactgacttcctcttcctgttgggcaaacttctgatcgttggtagtgtggggatcctggctttcttcttcttcacccaccgtatcaggatcgtgcaggatacagcaccacccctcaattattactgggttcctatactgacggtgatcgttggctcctacttgattgcacacggtttcttcagcgtctatggcatgtgtgtggacacgctgttcctctgcttctgtgagtgacccctcaccccaaaccttgctgggccccgaatcccttctttccactgggcatcacatcaccctccaacggggcaacacgcttgcctgcccccagcttccccagggcttggctgtccctcgtcctgggtccccagcctgtcttcctggtttccttttgcgcttagaagcagctccgacctcctgtccactggcccaggctgcagcctggacgctgccctggagcccgcccgcgcttcgcagtttctggctttgactggggggaggggatctgtggctgccactaactctggtctctccatctgttttttttgtttgtttttttcttctctcttcctctcctccatgcctgctggcttccctgttcttccctgcctccctctttccctcccttcccgaccaccccattttccccctgccggttcccggggggagcccaggtgaggacctggaaaggaatgacggctctcaggagcgaccctacttcatgtcgcccgagctgagagacatcctgttgaaggggagtgcggaggaggggaagcgggcagaagccgaggagtagagagtgagggagactggcgtgggggccaggtttcctccatgtagactgggggtgcatgaagcgggggggttcctggcctgcgagtgtggggatcctgtgtgtccctcggagcccactacagtctgcccctctctggtcccagtgtgtctgctttctaaccctctgaggcttctctgtgaccctcatccacctaccctgtccttgaggcccctgcccgtgggctcccctcatgcctcctgctctgggacctctctccacagtggaggacctggagaggaatgacggctcggccgagaggccttacttcatgtcttccaccctcaagaaactcttgaacaagaccaacaagaaggcagcggagtcctgaaggccccgtgctccccacctctcaaggagtctcatgccgcagggtgctcagtagctgggtctgttcccccagccccttgggctcacctgaagtcctatcactgccgctctgcccctccccatgagccagatcccaccagtttctggacgtggagagtctggggcatctccttcttatgccaaggggcgcttggagttttcatggctgcccctccagactgcgagaaacaagtaaaaacccattggggcctcttgatgtctgggatggcacgtggcccgacctccacaagctccctcatgcttcctgtcccccgcttacacgacaacgggccagaccacgggaaggacggtgtttgtgtctgagggagctgctggccacagtgaacacccacgtttattcctgcctgctccggccaggactgaaccccttctccacacctgaacagttggctcaagggccaccagaagcatttctttattattattattttttaacctggacatgcattaaagggtctattagctttcaaaaaaaaaaaaaaaaaaaaaaaaaaaaaaaaaaaaaaaaaaaaaaaaaaaaaaaaaaaaaaaaaaaaaaaa

SEQ ID NO: 204 - protein, 373 aa, Homo sapiens
MRPWREWGAQPAQPLPFAVIILSILEVIIILLLIFLRKRILIAIALIKEASRAVGYVMCSLLYPLVTFFLLCLCTAYWASTAVFLSTSNEAVYKIFDDSPCPFTAKTCNPETFPSSNESRQCPNARCQFAFYGGESGYHRALLGLQIFNAFMFFWLANFVLALGQVTLAGAFASYYWALRKPDDLPAFPLFSAFGRALRYHTGSLAFGALILAIVQIIRVILEYLDQRLKAAENKFAKCLMTCLKCCFWCLEKFIKFLNRNAYIMIAIYGTNFCTSARNAFFLLMRNIIRVAVLDKVTDFLFLLGKLLIVGSVGILAFFFFTHRIRIVQDTAPPLNYYWVPILTVIVGSYLIAHGFFSVYGMCVDTLFLCFCE

SEQ ID NO: 205 - DNA, 422 bp, Homo sapiens
  unsure (67)
  unsure (148)
  unsure (206)
  unsure (230)
  unsure (234)
  unsure (254)
tttttttttgctccagcaccagtgttccctaaacaccttaccagcagcttccattttggcatggaanagtgttctcggcaatggccatttgtcatcactgggaaccagagacacccatccctacgccagcttgagccgtgcactgcanacacaatgctgtatttcttctcccagtcacctgatgagccagcagtatagaccatatngtttcttcactaaattgactgcanatgagctgtggaaaggcgctttagcagagactggtgctggagcaaaaaaaggaagaggccaaagaactaaaaagaagaaaagaaaggatctgaacaggggtcanatcattggtgaagggcgttntggttttctatggcccggactgaatgtccctccttatgaaaaatggagcagtgcagaccattgccccc

SEQ ID NO: 206 - DNA, 515 bp, Homo sapiens
gcaaccacaatgggcagaggcaacattcttcccggatttccacaacatggaggccctttcttatcagccagctgttgatgggtttcctgtctggagagcccacggaagaggccctgggtgaggctgagcatattaatggacccagagaccttggcatacatgtctttgatgccaatgagccggcagatggtgatgatggccctgtggcagcggaggccgtaacctttgggttgtttcttcatcttgatatgcgtccttttaaatcttaatgaaatatcatggaatattgtatggtcttcatatcgttctatataatgcaaatggtgaactgctctgttctttgctttcctgaaagcatccatccgatcagtagctttcccaatagaaaaacctgcagctccttttccgttccccacagccaccaagacacggatcgatttctttcttccctctttcgcagtcatagtgaaaacgtttcttacctcaagtatcctggtatcaaaatcctcatatgt

SEQ ID NO: 207 - protein, 79 aa, Homo sapiens
MEYCMVFISFYIMQMVNCSVLCFPESIHPISSFPNRKTCSSFSVPHSHQDTDRFLSSLFRSHSENVSYLKYPGIKILIC

SEQ ID NO: 208 - DNA, 144 bp, Homo sapiens
  unsure (11)..(12)
  unsure (14)..(15)
aaaaaaaaaanntnntttttttaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 209 - DNA, 1243 bp, Homo sapiens
tgcttctctgaagacttgcagcaaggcttgctgaggctcacagaagatagccccagtgttttggagtggttttgaatgtgattctgagatcagactgactgagctggaatcctggctttatatcttaccagctacacaaccttggagtcttagaaattttttcttttcaataagcagtcatccttactttccctcaagatgacaaacagttcgttcttctgcccagtttataaagatctggagccattcacgtattttttttatttagttttccttgttggaattattggaagttgttttgcaacctgggcttttatacagaagaatacgaatcacaggtgtgtgagcatctacttaattaatttgcttacagccgatttcctgcttactctggcattaccagtgaaaattgttgttgacttgggtgtggcaccttggaagctgaagatattccactgccaagtaacagcctgcctcatctatatcaatatgtatttatcaattatcttcttagcatttgtcagcattgaccgctgtcttcagctgacacacagctgcaagatctaccgaatacaagaacccggatttgccaaaatgatatcaaccgttgtgtggctaatggtccttcttataatggtgccaaatatgatgattcccatcaaagacatcaaggaaaagtcaaatgtgggttgtatggagtttaaaaaggaatttggaagaaattggcatttgctgacaaatttcatatgtgtagcaatatttttaaatttctcagccatcattttaatatccaattgccttgtaattcgacagctctacagaaacaaagataatgaaaattacccaaatgtgaaaaaggctctcatcaacatacttttagtgaccacgggctacatcatatgctttgttccttaccacattgtccgaatcccgtataccctcagccagacagaagtcataactgattgctcaaccaggatttcactcttcaaagccaaagaggctacactgctcctggctgtgtcgaacctgtgctttgatcctatcctgtactatcacctctcaaaagcattccgctcaaaggtcactgagacttttgcctcacctaaagagaccaaggctcagaaagaaaaattaagatgtgaaaataatgcataaaagacaggattttttgtgctaccaattctggccttactggaccataaagttaattatagctttgaaagataaaaaaaaaaaaaaa

SEQ ID NO: 210 - protein, 319 aa, Homo sapiens
MTNSSFFCPVYKDLEPFTYFFYLVFLVGIIGSCFATWAFIQKNTNHRCVSIYLINLLTADFLLTLALPVKIVVDLGVAPWKLKIFHCQVTACLIYINMYLSIIFLAFVSIDRCLQLTHSCKIYRIQEPGFAKMISTVVWLMVLLIMVPNMMIPIKDIKEKSNVGCMEFKKEFGRNWHLLTNFICVAIFLNFSAIILISNCLVIRQLYRNKDNENYPNVKKALINILLVTTGYIICFVPYHIVRIPYTLSQTEVITDCSTRISLFKAKEATLLLAVSNLCFDPILYYHLSKAFRSKVTETFASPKETKAQKEKLRCENNA

SEQ ID NO: 211 - DNA, 2254 bp, Homo sapiens
ggtttgaaaactctgcttcctttgtgaatttggtgttaggagttcttattgttattctgcagcctttactattgtcctttatttactgaacacagtgaataccaagcactgtttattagaggttaggagtaggggcaggtgattaaagaaacaaaaaagctaataatctcctcaaacaatttctggcctaatagaattatagtagacagtgaagtatctaaacccagggaatcagattgaggcaccatgtccatcgccttgagaattaataggctgcatttctgggttctcctttttttttttttttttgcccaactgagtctttctgtggacttacatggaacttcttattctcttaaatcattaagttacttgacaatattcttggatttggagaaactggatgtagggccgtatgaaaaaatcattcgaaatcagatttaggggtataaggttggataggaatgttttagaaagaagaatgtaaggcagataactaatttgtcacatccaaagtataaaactgctactttttccctagaaaagggaagctcattttaggcagcctaaaccagtaagattttcttcctcctccaagtgcagatttttgtacctttcgtttgtcaaaacattctttggccctatgcatgccagagtgatatagaaaggaagttaccacatttttttgagaacaaatcactcctgataaaatttcttagacaattgataatcattttaagaagaaatttaattgtatttagctctgtgtctcgcccctttggtgtcactcttctacctcttccatcactatagctaaatatttagaagtatatcttgacacctagcacaaatgttttggttaagtatcttaaaactgatggatggtatggctggggcagcatggctcacgcctgtaatcccagcactttgggaggccaaggcgggtgaatcacctgaggtcaggagtttgagaccggcctgaccaacttggagaaaccccgtctctactaaaaatacaaaaattagtcaggggtggtggcgcatgcctgtgatcctgtctactcgggaggctgaggcaggataattgcctgaacccgggaggcagaggttgcagtgagctgagatcgtgccattgcactccagcctgggcaacaagagcaaaactcagtctcaaaaaacaaaacaaaaaacctgttggtatagtacgaaagaaacgtcttgcagttttctgttgcagagaattaattagaaccaacctgttggattatacacattcacctttcagaatcctttcttctctgtggaaacccacactctcagcagtgtgtgggaacacagtagattcttaaggaatgcttgttgaatgttgcagtctgcatcttcttgaagtaacagaactgttggtagctgtttaaaagtaaaatgtgtctaaagaccttttggaaattaagatgtaagagattaatgcaccaaagcagtctcttaattacttaaatgaattatttcaaagaatctttaattgaattttctgtgaagtctggaatttgtaaattatgtccctttgttcaaaccagcccctgaaaagaacaattaaggcaattaagatagcattaaagttttcaatgaagttggcattttccgtgtattaagattagatgttagctgctgaagtttgtggaggtcggacataaagcttccaacatcagtaatgcaaaattgtcttgaacctgcgataaaattttgttggacttttttttcattgcagtgaaaagggccatgtagcatgcctcaaagccaggttactcagcctagtccttgtttaagcagttttgatattcattcaagttcaattttctcactgattttatgattaatttcttggaaaatttgaaagttttcaagaagtaaaaaattaatatcttgttatcccagtatatagagattaacttcagttcaatgtttggtgcattttcttctagtctttttttcaatgtataaatattaaagttatttcatagttgagatcatactgcatatatgatactgtatcttgcttttttcattttaacgtcgtgagcaattttcccatgacattacaaactgtctttgaaaaatggaaaacatttggggctgtcagcataactgaaaatgttttcttggtgtgacacatgtatctttgtaattggtttgatttagtgtgctttatttcaataaaaattcagtattataatttaaaaaaaaaaaaaaaaaaaaa

SEQ ID NO: 212 - protein, 65 aa, Homo sapiens [sequence omitted]

SEQ ID NO: 213 - DNA, 2975 bp, Homo sapiens
gagcgacgcggagaagagctccgggtgccgcggcactgcagcgctgagattcctttacaaagaaactcagaggaccgggaagaaagaatttcacctttgcgacgtgctagaaaataaggtcgtctgggaaaaggactggagacacaagcgcatccaaccccggtagcaaactgatgacttttccgtgctgatttctttcaacctcggtattttcccttggatattaacttgcatatctgaagaaatggcattccggacaatttgcgtgttggttggagtatttatttgttctatctgtgtgaaaggatcttcccagccccaagcaagagtttatttaacatttgatgaacttcgagaaaccaagacctctgaatacttcagcctttcccaccatcctttagactacaggattttattaatggatgaagatcaggaccggatatatgtgggaagcaaagatcacattctttccctgaatattaacaatataagtcaagaagctttgagtgttttctggccagcatctacaatcaaagttgaagaatgcaaaatggctggcaaagatcccacacacggctgtgggaactttgtccgtgtaattcagactttcaatcgcacacatttgtatgtctgtgggagtggcgctttcagtcctgtctgtacttacttgaacagagggaggagatcagaggaccaagttttcatgattgactccaagtgtgaatctggaaaaggacgctgctctttcaaccccaacgtgaacacggtgtctgttatgatcaatgaggagcttttctctggaatgtatatagatttcatggggacagatgctgctatttttcgaagtttaaccaagaggaatgcggtcagaactgatcaacataattccaaatggctaagtgaacctatgtttgtagatgcacatgtcatcccagatggtactgatccaaatgatgctaaggtgtacttcttcttcaaagaaaaactgactgacaataacaggagcacgaaacagattcattccatgattgctcgaatatgtcctaatgacactggtggactgcgtagccttgtcaacaagtggaccactttcttaaaggcgaggctggtgtgctcggtaacagatgaagacggcccagaaacacactttgatgaattagaggatgtgtttctgctggaaactgataacccgaggacaacactagtgtatggcatttttacaacatcaagctcagttttcaaaggatcagccgtgtgtgtgtatcatttatctgatatacagactgtgtttaatgggccttttgcccacaaagaagggcccaatcatcagctgatttcctatcagggcagaattccatatcctcgccctggaacttgtccaggaggagcatttacacccaatatgcgaaccaccaaggagttcccagatgatgttgtcacttttattcggaaccatcctctcatgtacaattccatctacccaatccacaaaaggcctttgattgttcgtattggcactgactacaagtacacaaagatagctgtggatcgagtgaacgctgctgatgggagataccatgtcctgtttctcggaacagatcggggtactgtgcaaaaagtggttgttcttcctactaacaactctgtcagtggcgagctcattctggaggagctggaagtctttaagaatcatgctcctataacaacaatgaaaatttcatctaaaaagcaacagttgtatgtgagttccaatgaaggggtttcccaggtatctctgcaccgctgccacatctatggtacagcctgtgctgactgctgcctggcgcgggacccttattgcgcctgggatggccattcctgttccagattctacccaactgggaaacggaggagccgaagacaagatgtgagacatggaaacccactgactcaatgcagaggatttaatctaaaagcatacagaaatgcagctgaaattgtgcagtatggagtaaaaaataacaccacttttctggagtgtgcccccaagtctccgcaggcatctatcaagtggctgttacagaaagacaaagacaggaggaaagaggttaagctgaatgaacgaataatagccacttcacagggactcctgatccgctctgttcagggttctgaccaaggactttatcactgcattgctacagaaaatagtttcaagcagaccatagccaagatcaacttcaaagttttagattcagaaatggtggctgttgtgacggacaaatggtccccgtggacctgggccagctctgtgagggctttacccttccacccgaaggacatcatgggggcattcagccactcagaaatgcagatgattaaccaatactgcaaagacactcggcagcaacatcagcagggagatgaatcacagaaaatgagaggggactatggcaagttaaaggccctcatcaatagtcggaaaagtagaaacaggaggaatcagttgccagagtcataatattttcttatgtgggtcttatgcttccattaacaaatgctctgtcttcaatgatcaaattttgagcaaagaaacttgtgctttaccaaggggaattactgaaaaaggtgattactcctgaagtgagttttacacgaactgaaatgagcatgcattttcttgtatgatagtgactagcactagacatgtcatggtcytcatggtgcatataaatatatttaacttaacccagattttatttatatctttattcaccttttcttcaaaatcgatatggtggctgcaaaactagaattgttgcatccctcaattgaatgagggccatatccctgtggtattcctttcctgctttggggctttagaattctaattgtcagtgattttgtatatgaaaacaagttccaaatccacagcttttacgtagtaaaagtcataaatgcatatgacagaatggctatcaaaagaaaaaaaaaaa

SEQ ID NO: 214 - protein, 751 aa, Homo sapiens
MAFRTICVLVGVFICSICVKGSSQPQARVYLTFDELRETKTSEYFSLSHHPLDYRILLMDEDQDRIYVGSKDHILSLNINNISQEALSVFWPASTIKVEECKMAGKDPTHGCGNFVRVIQTFNRTHLYVCGSGAFSPVCTYLNRGRRSEDQVFMIDSKCESGKGRCSFNPNVNTVSVMINEELFSGMYIDFMGTDAAIFRSLTKRNAVRTDQHNSKWLSEPMFVDAHVIPDGTDPNDAKVYFFFKEKLTDNNRSTKQIHSMIARICPNDTGGLRSLVNKWTTFLKARLVCSVTDEDGPETHFDELEDVFLLETDNPRTTLVYGIFTTSSSVFKGSAVCVYHLSDIQTVFNGPFAHKEGPNHQLISYQGRIPYPRPGTCPGGAFTPNMRTTKEFPDDVVTFIRNHPLMYNSIYPIHKRPLIVRIGTDYKYTKIAVDRVNAADGRYHVLFLGTDRGTVQKVVVLPTNNSVSGELILEELEVFKNHAPITTMKISSKKQQLYVSSNEGVSQVSLHRCHIYGTACADCCLARDPYCAWDGHSCSRFYPTGKRRSRRQDVRHGNPLTQCRGFNLKAYRNAAEIVQYGVKNNTTFLECAPKSPQASIKWLLQKDKDRRKEVKLNERIIATSQGLLIRSVQGSDQGLYHCIATENSFKQTIAKINFKVLDSEMVAVVTDKWSPWTWASSVRALPFHPKDIMGAFSHSEMQMINQYCKDTRQQHQQGDESQKMRGDYGKLKALINSRKSRNRRNQLPES

SEQ ID NO: 215 - DNA, 447 bp, Homo sapiens
ggcgaaggcagcggcaggtcgggagcaagatggcgctgcggccaggagctggttctggtggcggcggggccgcgggagctggcgcggggtccgccgggggaggcggcttcatgtttcctgttgcaggtgggataagaccccctcaagcaggcctgatgccgatgcagcaacaaggatttcctatggtctctgtcatgcagcctaatatgcaaggcattatgggaatgaattacagctctcagatgtcccaaggacctattgctatgcaggcaggaataccaatgggaccaatgccagcagcgggaatgccttacctaggacaagcacccttcctgggcatgcgtcctccaggcccacagtacactccagacatgcagaagcagtttgccgaagagcagcagaaacgatttgaacagcagcaaaaactcttagaaaaaaaaaaaaaaa

SEQ ID NO: 216 - protein, 135 aa, Homo sapiens
MALRPGAGSGGGGAAGAGAGSAGGGGFMFPVAGGIRPPQAGLMPMQQQGFPMVSVMQPNMQGIMGMNYSSQMSQGPIAMQAGIPMGPMPAAGMPYLGQAPFLGMRPPGPQYTPDMQKQFAEEQQKRFEQQQKLLE

SEQ ID NO: 217 - DNA, 28 bp, Artificial Sequence
  oligonucleotide
cacaggcatacacaggaagatacattca

SEQ ID NO: 218 - DNA, 25 bp, Artificial Sequence
  oligonucleotide
tcttgctggatgggaacggaattca

SEQ ID NO: 219 - DNA, 28 bp, Artificial Sequence
  oligonucleotide
atacattcacagaagagcttcctgcaca

SEQ ID NO: 220 - DNA, 28 bp, Artificial Sequence
  oligonucleotide
ttctctgctgtctctccttcatcctgtt

SEQ ID NO: 221 - DNA, 25 bp, Artificial Sequence
  oligonucleotide
gggcctgaggttgaactggggtgaa

SEQ ID NO: 222 - DNA, 27 bp, Artificial Sequence
  oligonucleotide
ccatcagtatgtcttcgagctgcatgc

SEQ ID NO: 223 - DNA, 45 bp, Artificial Sequence
  oligonucleotide
cacagtggaaagcctgtaggacatcaaaaggagcttctctaccac

SEQ ID NO: 224 - DNA, 27 bp, Artificial Sequence
  oligonucleotide
gtggttttaggcgcacactctctctca

SEQ ID NO: 225 - DNA, 29 bp, Artificial Sequence
  oligonucleotide
catgtttctgacaatcatagtgtgtggaa

SEQ ID NO: 226 - DNA, 29 bp, Artificial Sequence
  oligonucleotide
ttccttgggcctgaatgacttgaatgttt

SEQ ID NO: 227 - DNA, 28 bp, Artificial Sequence
  oligonucleotide
ttatgcccctatgattggattggtttcc

SEQ ID NO: 228 - DNA, 27 bp, Artificial Sequence
  oligonucleotide
gctcaggaatgggaaagggaactggga

SEQ ID NO: 229 - DNA, 30 bp, Artificial Sequence
  oligonucleotide
catgataatagcttttctaactgtgggtac

SEQ ID NO: 230 - DNA, 45 bp, Artificial Sequence
  oligonucleotide
aaaggcagtaaatcagccacaaataaggaacttggtggttaaaca

SEQ ID NO: 231 - DNA, 27 bp, Artificial Sequence
  oligonucleotide
ttgatgctgtgctcatcctcctgctca

SEQ ID NO: 232 - DNA, 27 bp, Artificial Sequence
  oligonucleotide
gaaataataatggtggtggtggtgcgg

SEQ ID NO: 233 - DNA, 27 bp, Artificial Sequence
  oligonucleotide
atgctcatgaacccaatccggagaagg

SEQ ID NO: 234 - DNA, 29 bp, Artificial Sequence
  oligonucleotide
ttgattatcttcaccatcaaggccaacag

SEQ ID NO: 235 - DNA, 30 bp, Artificial Sequence
  oligonucleotide
ggaaatgacttttgatgacctaaagatcca

SEQ ID NO: 236 - DNA, 28 bp, Artificial Sequence
  oligonucleotide
tgagatgaacagaagacccaaacatagc

SEQ ID NO: 237 - DNA, 27 bp, Artificial Sequence
  oligonucleotide
tctcacgccaaagctcacaccttcagc

SEQ ID NO: 238 - DNA, 28 bp, Artificial Sequence
  oligonucleotide
ttgggtcttttgcataatgatcgtcttc

SEQ ID NO: 239 - DNA, 28 bp, Artificial Sequence
  oligonucleotide
gttgtatttcaaagagcagtagcaaatc

SEQ ID NO: 240 - DNA, 28 bp, Artificial Sequence
  oligonucleotide
gaactggccttcatagggcaacacattt

SEQ ID NO: 241 - DNA, 28 bp, Artificial Sequence
  oligonucleotide
aaccccgaggagaactgctaccatagaa

SEQ ID NO: 242 - DNA, 28 bp, Artificial Sequence
  oligonucleotide
acaagatgaaattgatagcaaatgcgac

SEQ ID NO: 243 - DNA, 28 bp, Artificial Sequence
  oligonucleotide
tctcgatggcccattctgcaaggtagag

SEQ ID NO: 244 - DNA, 28 bp, Artificial Sequence
  oligonucleotide
tatcaaggcagttgcttctactcctggg

SEQ ID NO: 245 - DNA, 28 bp, Artificial Sequence
  oligonucleotide
ggtttccctagttgagtcctcaggtcct

SEQ ID NO: 246 - DNA, 28 bp, Artificial Sequence
  oligonucleotide
aatgatcaggatctaagtgttaggcgga

SEQ ID NO: 247 - DNA, 27 bp, Artificial Sequence
  oligonucleotide
acctgctggagtcagccaagatgttta

SEQ ID NO: 248 - DNA, 26 bp, Artificial Sequence
  oligonucleotide
gaatgggactccaagcctgcctccta

SEQ ID NO: 249 - DNA, 29 bp, Artificial Sequence
  oligonucleotide
tnttcctttatgctgcaagtggtgacatg

SEQ ID NO: 250 - DNA, 29 bp, Artificial Sequence
  oligonucleotide
tnctactggaaatgttggtcctgtatgac

SEQ ID NO: 251 - DNA, 29 bp, Artificial Sequence
  oligonucleotide
gncgcatcatcgctcagacaattataatg

SEQ ID NO: 252 - DNA, 29 bp, Artificial Sequence
  oligonucleotide
gnctgacccactcctttatttgggtatgc

SEQ ID NO: 253 - DNA, 29 bp, Artificial Sequence
  oligonucleotide
tnaagttctaggcacttaaccagcgatac

SEQ ID NO: 254 - DNA, 29 bp, Artificial Sequence
  oligonucleotide
cnggctcttccagttacaaggcatataaa

SEQ ID NO: 255 - DNA, 30 bp, Artificial Sequence
  oligonucleotide
agtgtagatacaatcagtttaaatgctgca

SEQ ID NO: 256 - DNA, 34 bp, Artificial Sequence
  oligonucleotide
attttcatatttttaaaaatatgctatatcatgg

SEQ ID NO: 257 - DNA, 29 bp, Artificial Sequence
  oligonucleotide
antatcccaccagcttctcacaggtgtca

SEQ ID NO: 258 - DNA, 29 bp, Artificial Sequence
  oligonucleotide
gnaggcatcactgtggctatttcaatctc

SEQ ID NO: 259 - DNA, 29 bp, Artificial Sequence
  oligonucleotide
tnccatctaccgcaactctcagttcgaga

SEQ ID NO: 260 - DNA, 29 bp, Artificial Sequence
  oligonucleotide
tnagtgtagtgacagtggtgacatccttt

SEQ ID NO: 261 - DNA, 27 bp, Artificial Sequence
  oligonucleotide
tgaaggggtctgaaaagggcagatgag

SEQ ID NO: 262 - DNA, 29 bp, Artificial Sequence
  oligonucleotide
tnttggtggagatgccatccggaacctca

SEQ ID NO: 263 - DNA, 29 bp, Artificial Sequence
  oligonucleotide
gntcttgggatgcgtcgccctgcagataa

SEQ ID NO: 264 - DNA, 29 bp, Artificial Sequence
  oligonucleotide
gnaagccgacaagtttcgtcttcctataa

SEQ ID NO: 265 - DNA, 29 bp, Artificial Sequence
  oligonucleotide
gntaaaccaaggtggtagtaatggagtgg

SEQ ID NO: 266 - DNA, 29 bp, Artificial Sequence
  oligonucleotide
gnttggtggaggtgacggagttgaaagag

SEQ ID NO: 267 - DNA, 29 bp, Artificial Sequence
  oligonucleotide
gnctcctccagaaattctggatcaatgac

SEQ ID NO: 268 - DNA, 27 bp, Artificial Sequence
  oligonucleotide
tcctcctacttctcctcttgacagcga

SEQ ID NO: 269 - DNA, 29 bp, Artificial Sequence
  oligonucleotide
tnccttagaaacatgactgcggtgacaat

SEQ ID NO: 270 - DNA, 29 bp, Artificial Sequence
  oligonucleotide
tnagatccaacagtcacgttcacgaaacc

SEQ ID NO: 271 - DNA, 29 bp, Artificial Sequence
  oligonucleotide
cntcctggttgttgtttgaagagcaggcg

SEQ ID NO: 272 - DNA, 28 bp, Artificial Sequence
  oligonucleotide
tngcccaagaaactgggtttcacattta

SEQ ID NO: 273 - DNA, 29 bp, Artificial Sequence
  oligonucleotide
gntgaagcatgcccaatttcatttcctct

SEQ ID NO: 274 - DNA, 29 bp, Artificial Sequence
  oligonucleotide
antgttctctggcttgtcagggaagactg

SEQ ID NO: 275 - DNA, 29 bp, Artificial Sequence
  oligonucleotide
tncaagttgagttgtacagaagcccaaga

SEQ ID NO: 276 - DNA, 29 bp, Artificial Sequence
  oligonucleotide
gntgtgagaagaccactcggtgatgacct

SEQ ID NO: 277 - DNA, 29 bp, Artificial Sequence
  oligonucleotide
tngagtctgggtggtagacaaatcatgca

SEQ ID NO: 278 - DNA, 29 bp, Artificial Sequence
  oligonucleotide
anggacggtatatatcaccatgaacaagt

SEQ ID NO: 279 - DNA, 29 bp, Artificial Sequence
  oligonucleotide
anaggcaggaggagacgggattgatggtt

SEQ ID NO: 280 - DNA, 29 bp, Artificial Sequence
  oligonucleotide
anaagcgtcatgcagagccatgatgaggg

SEQ ID NO: 281 - DNA, 29 bp, Artificial Sequence
  oligonucleotide
anaaatgtagcaggcttggcttgcagcag

SEQ ID NO: 282 - DNA, 29 bp, Artificial Sequence
  oligonucleotide
angacccatttccagtccaaatctttgac

SEQ ID NO: 283 - DNA, 29 bp, Artificial Sequence
  oligonucleotide
gncaaggtgtctgtaggagaaactatata

SEQ ID NO: 284 - DNA, 29 bp, Artificial Sequence
  oligonucleotide
anccagggctatacagagaaaccctgtct

SEQ ID NO: 285 - DNA, 29 bp, Artificial Sequence
  oligonucleotide
gntcttgaagaagtagcccagggtgagga

SEQ ID NO: 286 - DNA, 29 bp, Artificial Sequence
  oligonucleotide
cnggtaaaggtgatgttgacactgtagga

SEQ ID NO: 287 - DNA, 29 bp, Artificial Sequence
  oligonucleotide
tntggaacatactggatttaaagaacagg

SEQ ID NO: 288 - DNA, 29 bp, Artificial Sequence
  oligonucleotide
anagtttaaggaaagccacaagcacggat

SEQ ID NO: 289 - DNA, 29 bp, Artificial Sequence
  oligonucleotide
tncgttcctctattcggtgatggcactcc

SEQ ID NO: 290 - DNA, 29 bp, Artificial Sequence
  oligonucleotide
anagagcaccactatcaagtagctgacgt

SEQ ID NO: 291 - DNA, 29 bp, Artificial Sequence
  oligonucleotide
angaagagatgcatttccatcagtctggg

SEQ ID NO: 292 - DNA, 29 bp, Artificial Sequence
  oligonucleotide
anatctgaactgaagccaagttgtgcctc

SEQ ID NO: 293 - DNA, 29 bp, Artificial Sequence
  oligonucleotide
gnctcatgcacttcagttggctgcccact

SEQ ID NO: 294 - DNA, 29 bp, Artificial Sequence
  oligonucleotide
angcctgtcacaatactgtagtggtttgg

SEQ ID NO: 295 - DNA, 29 bp, Artificial Sequence
  oligonucleotide
gngcaataccccaccagcaaccaagattc

SEQ ID NO: 296 - DNA, 29 bp, Artificial Sequence
  oligonucleotide
tntgtcctgaaagagctcagtactcagct

SEQ ID NO: 297 - DNA, 28 bp, Artificial Sequence
  oligonucleotide
tgtggattatagtcttcgatgtgagcag

SEQ ID NO: 298 - DNA, 29 bp, Artificial Sequence
  oligonucleotide
anaacacgtcctttcatgccacggctgag

SEQ ID NO: 299 - DNA, 29 bp, Artificial Sequence
  oligonucleotide
gntgctccatttccttgatcataatgctc

SEQ ID NO: 300 - DNA, 31 bp, Artificial Sequence
  oligonucleotide
anagggtagaacgtggagaagaacatggtgt

SEQ ID NO: 301 - DNA, 29 bp, Artificial Sequence
  oligonucleotide
tntggttccatctgtgtaaatcttcccgg

SEQ ID NO: 302 - DNA, 29 bp, Artificial Sequence
  oligonucleotide
gncacaaactcagcatgcagggctggctt

SEQ ID NO: 303 - DNA, 29 bp, Artificial Sequence
  oligonucleotide
antcacatcagactctggaattgggtgaa

SEQ ID NO: 304 - DNA, 32 bp, Artificial Sequence
  oligonucleotide
gnntaaaacttgttctctgcagctttcagccg

SEQ ID NO: 305 - DNA, 29 bp, Artificial Sequence
  oligonucleotide
gncattatatagaacgatatgaagaccat

SEQ ID NO: 306 - DNA, 26 bp, Artificial Sequence
  oligonucleotide
gctgacacacagctgcaagatctacc

SEQ ID NO: 307 - DNA, 29 bp, Artificial Sequence
  oligonucleotide
gngcctcaatctgattccctgggtttaga

SEQ ID NO: 308 - DNA, 29 bp, Artificial Sequence
  oligonucleotide
gnccggaatgccatttcttcagatatgca

SEQ ID NO: 309 - DNA, 29 bp, Artificial Sequence
  oligonucleotide
tnccattggtattcctgcctgcatagcaa

SEQ ID NO: 310 - protein, 191 aa, Homo sapiens
MAAVTHYLYLCQFSWMLIQSVNFWYVLVMNDEHTERRYLLFFLLSWGLPAFVVILLIVILKGIYHQSMSQIYGLIHGDLCFIPNVYAALFTAALVPLTCLVVVFVVFIHAYQVKPQWKAYDDVFRGRTNAAEIPLILYLFALISVTWLWGGLTTWPTDTSGCWFSLSFSTVCRDFMFSWFISFYTTKCVAL

SEQ ID NO: 311 - DNA, 2412 bp, Homo sapiens
aagccccccacgccccgagcccaccctgctcaccggcctctgcccgagttccccgcatagtgtgggagtgtggagcatcctagcttttccccagcgcccagttctttcactctcactggagtcctgcagggacagctcgggcaccatgtaggcccgggtgggcgtgggggctcacctacctcggtggtgaacagctggcacgtctctgggttgcggacggtaaaggccacgtatacctcaggagcccgcttgtgctcccggcaggcagccagcctctgcaggaccccgaccagcgacacgatggcttctgggcaatacatcacgtctacagtgaaagtttcaggttactgaaagggacaagtggaaagttccatttcatgctgacctcagcagcagggcgaggccagagaggcagcggtcatatgagactattagatgccatttgaccatttgggccattagatggaaaggcaattatttgggtgaaaaaggagaacccttattagagaaagctgcaaaagaccgaagcaaaagaaaaaaatctccagactcactggtgtccttaaaaaaccagctctggttctcggcctgtctagagggctttcaatgacagaaagcctgaccctgccgtgaacttcgtgtttcaggtgtctgccgattggtctgctggcttgcaggggtgggcctgtgtccctggccaccgctggacctgtgggtttcagggctgggacccaggactacaggcagagctctgttccaccagagaggggactgagtgtgctcacaggggtgaggggtttttggtggcccagccaaacagcaccttctctcaagggccctgacctcgtaccaaaagtggttgttttcctcctgcggtctctgaaggacacagggcatggctctgggacagagccatgtggtgacgactgtaacgggagtatgcctgtctccaacaacagggctgtggcttgaaggtcaccttaagaggcacccctgtcctttgatgtcaccctggaggcccagagtaactcttctggaagccccatcacgtccatgcccgacagtgtccattgttcccttttcccagagccaagagctgggtagagctgcaaggacaccgcctgcacaggatgcccggggctgggcattacctgctgcaatgacaacatctggctggaaggcagagagctgatggaccattgctacgtcccagtccagctgggccactgtcaccctggggctgtctaagttgccagtgatgtctgcctctaatgagaggccattgagaaggacattccctcggagctgctcgaggacccggctgtgagggtcgctgaagatgtatgcccgggggcggcacatcttgcagatggcaaggcctgtgaggccggcaccactgccaagctctaggacagtcctggcgggaggaaaggggaccgtgttttcgactgcaccagggtaagcctgcctcggtgccctgccctgtgccccgaggtcacctgttaatgaaggctgccgggttctcgatggcccattctgcaaggtagagggcggcatcccatgtgaccaggcctgtggtaccgtgggagatgatggctgtgctcttggagagtgtgaccgagcctcctgaggactgcaccaagagagggcgagagagtcagtccagcgatcagaaggcaagtggcttaraagacaagtagccatccaccacatggctgaataaaccatgacaggaccaatcgccactcagcaatgagaagcagctaactgttgacataccaacagttgcacgggcctcaagggtgtcacgcggcatgaaagacactcatctcaggccacacaggattccatctattgaacattcctgagacaacggaattctggcgatggagcacaggtcagtggtggccaggggccaggtgtggctatgaagaggtggctgccttgtgatgattcaatatgctatgtttttcctttgtggttttctgtatctatgttttatctcatttttttttttttttgagctctgtcacccaggctggagtcagtggcacgatcttggcttactgcagcatctgcctcctgggttcaagcagttctcctgcctcgcctgcccaggtagctgtgactgcaggcgtgtgccaccatgtctggctaatttttgtacttttttttgagacacagattcactctcgttgccgagattgtgccacagtaatccagcctgggcaacacagtgggactctgtctcaaaaaaaatatataaataaataaataaataaataaataaataaataaataaataagtaaaaaataaaatccatcctatttccaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 312 - protein, 98 aa, Homo sapiens
MALGQSHVVTTVTGVCLSPTTGLWLEGHLKRHPCPLMSPWRPRVTLLEAPSRPCPTVSIVPFSQSQELGRAARTPPAQDARGWALPAAMTTSGWKAES

SEQ ID NO: 313 - protein, 125 aa, Homo sapiens
MGRQDLRTQLGKLLHKKMAPQPRPTQAFPPTRNVGLRQAKLVSLAFPLVRLGLPALQPLTSCLKLLSPSSLRTVRMSQMRRRKKRKTVRNAARNTMATAVRRQRMRKMRMTPRRLKRTMKKKKKR

SEQ ID NO: 314 - DNA, 915 bp, Homo sapiens
  unsure (116)
caatgggggaaagtcataattcaacctgaaaacatggattgtaagaggaaataaaaaaatcaaacagtatgttttaagtttcccttttgatactgtgtttcagggtaagtgacagnttctgcaaaccaagactcagtcctgattataaaggatttttaaaattacattattaaaaatatgtatttattcttctttcactttatctattttccaaagcctctttcaagtaaactgtgaagtgcctgagtaccagtgaccatgacgtcacactttcttttatctcaagcactcattgttgtttgcatttgcccaaagtgctggctggctccgaaggccagttcccacaagaatgaattcggaaagacttcatccttgtgtttttccttctcttttaattccatgcaacgaggtcagcttttgcaacaaggtggggttttatttttttggtgcatgccatcaaatactctaacgagacatttttaatgaaagacttaaaccagataggccacaatgaaccaaattagaaatctgaacatgtcaccacttgcagcataaaggaatataaaagggcagagcaaagtcttttttcctaaggtgaatatttctaaggtaagtattcatttgtaaaagttttttttttccatcatgtctgaaaactttttaccacgcggtgagtattacaacaaaacatcccttggttaaaaaaaaaaaataccatcttgcaattcagcacacacctgcagctggtgtgctcatccaaaccaatcagtaggctaagagaatttaaaattccatacatatgagtttaggtattaatgccgattacacagtacacagtacagagggaggtccctatatccacacacacacacaccccatccagcatttacaccaaaagccttaccctttaaacac

SEQ ID NO: 315 - protein, 36 aa, Homo sapiens
MSLGINADYTVHSTEGGPYIHTHTHPIQHLHXKPYP

SEQ ID NO: 316 - protein, 21 aa, Homo sapiens [sequence omitted]

SEQ ID NO: 317 - DNA, 1475 bp, Homo sapiens
  unsure (1398)..(1399)
gtcgaccttgaagatgttttctaaagaatcaaaaatgctacaaagaagtatatcattttcaaatatggctttatcgtcttgtttacttttaccaggagatgccactgtcataacttcttcatgggataataatgtctatttttattccatagcatttggaagacgccaggacacgttaatgggacatgatgatgctgttagtaagatctgttggcatgacaacaggctatattctgcatcgtgggactctacagtgaaggtgtggtctggtgttcctgcagagatgccaggcaccaaaagacaccactttgacttgctggccgagctggaacatgatgtcagtgtagatacaatcagtttaaatgctgcaagcacactgttagtttccggcaccaaagaaggcacagtgaatatttgggacctcacaacggccaccttaatgcaccagattccatgccattcagggattgtatgtgacactgcttttagcccagatagtcgccatgtcctcagcacaggaacagatggctgtcttaatgtcattgatgtgcagacaggaatgctcatctcctccatgacatcagatgagccccagaggtgctttgtctgggatggaaattccgttttatctggcagtcagtctggtgaactgctcgtttgggacctccttggagcaaaaatcagtgagagaatacagggccacacaggtgctgtgacatgtatatggatgaatgaacagtgtagcagtatcatcacaggaggggaagacagacaaattatattctggaaattgcagtattaagtgccttttcctctcctgaatattaaattgaactctatttaatgcatttttaaaccaaacttttaaacggactggtgaatgtgcaatgttagtaattagaagttttaccacatggaaaatttgtggttttaaactttctaaatcatggtgacttcattgaaagccattagttgccattctcttagggcagataaaatgcggctgtgttaggaaaaacatgttacactgtaaggcagatgatcgtccccatatgatgattgtcagaagacaggactaagtagcagagaatagctaagagataaattgggctggggaaacttgtcagaaagcactgaacaattaagaaattttccaagaaaatgtgcagtattctctgctacttctgaatctgttttgtcttcctaatctatcacaattgccacccatcgggttttgggtgtgtgttttcatagcgtggttactttctataatgctgtacccagattctaagaacctggagaaggattagcagttcttagtaagtttactgtgtataggaacggtttgtatttcattacagctattcatcttttctacattaaaaatatttttctctnnaaaaaaaaaaaaaaaaaaaaaaaaaaaaaaaaaaaaaaaaaaaaaaaaaaaaaaaaaaaaaaaaaaaaaaaaaaaa

SEQ ID NO: 318 - protein, 262 aa, Homo sapiens
MFSKESKMLQRSISFSNMALSSCLLLPGDATVITSSWDNNVYFYSIAFGRRQDTLMGHDDAVSKICWHDNRLYSASWDSTVKVWSGVPAEMPGTKRHHFDLLAELEHDVSVDTISLNAASTLLVSGTKEGTVNIWDLTTATLMHQIPCHSGIVCDTAFSPDSRHVLSTGTDGCLNVIDVQTGMLISSMTSDEPQRCFVWDGNSVLSGSQSGELLVWDLLGAKISERIQGHTGAVTCIWMNEQCSSIITGGEDRQIIFWKLQY

SEQ ID NO: 319 - protein, 50 aa, Homo sapiens
NLKRCHPWRGLHQPVPQVTQDHPLLLLTAPRERPAFWATWPSGAQPLASR

SEQ ID NO: 320 - DNA, 399 bp, Homo sapiens
atgtggttatattctttgattaacttagtcctttttcttggtcaagagtcagtagacaggactgaagcttatgccccttgcccccccaccaccactccattactaccaccttggtttagccatcctttcttgatctgttctccccacttctactgtgctactctacagacttgccctgaatgtaagagcaacaattaccttgtaaagtccaagttggggcaggtcactcccaaactccacacctccagtcactgcagagttgagaagcctggggctgggaactatggaatcatttcctttctcacttcaacccctactcctctactcactgcccattctgctccagccacactggcctcttgctggtgttcacgtacatcaagcatacaccaaaggcct

SEQ ID NO: 321 - DNA, 1670 bp, Homo sapiens
cgccgacggcggccgagacggacatgaagcaatatcaaggctccggcggcgtcgccatggatgtggaacggagtcgcttcccctactgcgtggtgtggacgcccatcccggtgctcacgtggtttttccccatcatcggccacatgggcatctgcacatccacaggagtcattcgggacttcgcgggcccctactttgtctcagccgggagggcagtgtggccagaaggattcttaagtaactgacccagccctttgcccccacccctggggtaccgagacatgggtagggattagaggcaagagtggagagtcagaccatccaggaaccacatctctggaccttcagaaggaggacaacatggcctttggaaagcctgccaagtactggaagttggaccctgctcaggtctatgctagcgggcccaacgcatgggacacggctgtgcacgacgcctctgaggagtacaagcaccgcatgcacaatctctgctgtgacaactgccactcgcacgtggcatyggccctgaatctgatgcgctacaacaacagcaccaactggaatatggtgacgctctgcttcttctgcctgctctacgggaagtacgtcagcgttggggccttcgtgaagacctggctgcccttcatccttctcctgggcatcatcctcaccgtcagcctggtctttaacctccggtgatggctgctcggtggccccacacccaccagggtcccgaggaaacagccgccatcccttttggttccagatttttttctcctcaccccaaaaggcagggttgggcctgctgttgtggaccgggggtcggggctggcaggatggaaggactgaggaccagcatgaagtgggggtttgttgtctccctgcctctcagaagcaccctgtcccctcctccccaggcctgtgactccggccctggaagcccctttgttcttctgttgaaaggctttggcttccctctgtagagctgctcccgccaccacctgctggggtcctgcctcagcccagtgcccagtatggggagaggaggacatttgggctcacctgtcaaggtggccctgggaccagagctggtcccarcatggggtgcaccgggtacacttaacgtgtctctataarccaagttgcttcaggaccttcaccactggcctctagaatggtccagaggggctggctgggtccctttgtmagactcctgccggcagctkccctgggggacatgtgtgcccatctggcatcctccagcccgtgcagtccgctcttcactgttccacggcctcccagtgcctcccagcattggacccatctccccctgcagtttgaggccagagaggtgagtggacctgacaagtgccagagtaaccgtgtagacagagcagtgtagacagcgctcagccccagccccaggtgtggacctcatgctggtgatggctcccctgggtggcctgccagcacagccagtkccatcagggagctgaaggggctgtcccccacctaactccagctcccccttcacgttgtcaccaaggccctgtgccgcccgcctcgcccccctgctctgtggattcctttgggaagggctccctgggcaggacaataaagagttttgactccaaaaaaaaaaaaaaaaaaaaaaaaaaaaaaaaaaaaaa